(19) United States
(12) Patent Application Publication
(10) Pub. No.: US 2024/0376151 A1
(43) Pub. Date: Nov. 14, 2024

(54) STAPLED PEPTIDES AND METHODS THEREOF

(71) Applicant: FOG PHARMACEUTICALS, INC., Cambridge, MA (US)

(72) Inventors: Olena Tokareva, Andover, MA (US); Justin Robert Lawrie, Somerville, MA (US); Kunhua Li, Newton, MA (US); Tara Lynne Travaline, Arlington, MA (US); Jean-Marie Swiecicki, Somerville, MA (US); Gregory L. Verdine, Boston, MA (US); John Hanney McGee, Somerville, MA (US); Ty Matthew Thomson, Arlington, MA (US)

(21) Appl. No.: 18/497,597

(22) Filed: Oct. 30, 2023

Related U.S. Application Data
(60) Provisional application No. 63/453,464, filed on Mar. 20, 2023.
(60) Provisional application No. 63/421,159, filed on Oct. 31, 2022.

(51) Int. Cl. C07K 7/08; A61K 38/00; G01N 33/68

(52) U.S. Cl. CPC C07K 7/08; G01N 33/6818; A61K 38/00; C07K 2319/90

(57) ABSTRACT

The present disclosure provides powerful technologies for the development, production, characterization, and/or use of stapled peptide compositions. Among other things, the present disclosure provides strategies for defining amino acid sequences particularly amenable or useful for stapling, as well as technologies, reagents, and systems for developing, producing, characterizing, and/or using stapled peptides having such amino acid sequences. In some embodiments, the present disclosure provides stapled peptide agents and uses thereof including for treating various conditions, disorders or diseases.

### CROSS-REFERENCE TO RELATED APPLICATIONS

This application claims priority to United States Provisional Application Nos. 63/421,159, filed Oct. 31, 2022, and 63/453,464, filed Mar. 20, 2023, the entirety of each of which is incorporated herein by reference.

### SEQUENCE LISTING

The instant application contains a Sequence Listing which has been submitted electronically in XML file format and is hereby incorporated by reference in its entirety. Said XML copy, created on Jul. 19, 2024, is named SL.xml and is 1,107,601 bytes in size.

### BACKGROUND

Stapled peptides have been reported to have improved properties and activities relative to peptides that lack staples, and can be useful for various biological purposes.

### SUMMARY OF THE INVENTION

Among other things, the present disclosure provides powerful technologies for the development, production, characterization, and/or use of stapled peptide agents and compositions. In some embodiments, the present disclosure provides strategies for defining amino acid sequences particularly amenable or useful for stapling, as well as technologies, reagents, and systems for developing, producing, characterizing, and/or using stapled peptides having such amino acid sequences. In some embodiments, the present disclosure provides technologies for identifying stapled peptides useful for various purposes, e.g., binding to targets of interest, modulate activities of targets of interest, bridging targets of interest, etc.

Many stapling technologies develop useful and potent stapled peptides utilizing a known (and often naturally occurring) amino acid sequence. Among other things, the present disclosure identifies the source of a problem with such technologies that are necessarily constrained to pre-selected amino acid sequences. In some embodiments, the present disclosure encompasses the recognition that certain stapling technologies can be implemented to provide high-throughput generation and analysis of stapled peptides, including in the absence of predetermined sequence selection or bias. Thus, for example, in some embodiments, the present disclosure permits development of stapled peptides (and collections thereof) that bind to a particular target but that have amino acid sequence(s) that differ from (and, in some embodiments, lack significant identity to) that of a naturally occurring protein interaction partner(s) for the target, and in some embodiments, to a target that have no known protein interaction partners at all. In some embodiments, provided stapled peptides bind to targets different from natural interaction partners. In some embodiments, provided stapled peptides provided different biological outcomes from natural interaction partners. In some embodiments, provided technologies permit identification of amino acid sequences that show desirable binding characteristic(s) with respect to a particular target, and in particular show desirable binding characteristic(s) when provided in a stapled form. In some embodiments, the present disclosure thus provides peptide agents that bind to a target of interest, having an amino acid sequence identified, characterized, and/or produced as described herein; in some embodiments such peptide agents comprise stapled peptides.

As described herein, cysteine stapling strategies may be employed to facilitate production and/or characterization of stapled peptides in or by a biological system, e.g., a phage display system. In some embodiments, such production and/or characterization may comprise identification and/or selection of a useful amino acid sequence. In some embodiments, collections of stapled peptides are generated utilizing display technologies. Those skilled in the art, reading the present disclosure, will appreciate that, once such an amino acid sequence is identified and/or selected, a stapled peptide agent having such amino acid sequence may be prepared using any desired stapling technology. In some embodiments, stapled peptides and collections thereof are generated utilizing synthetic technologies. In some embodiments, amino acid residues at one or more positions are degenerate (e.g., being a set of amino acids such as natural amino acids without bias or enrichment for a relevant function or activity). In some embodiments, technologies such as mass spectrometry are utilized for identification and/or selection of useful amino acid sequences and stapled peptides.

In some embodiments, the present disclosure provides a collection of peptides (or nucleic acids that encode them) whose amino acid sequences show significant diversity relative to one another and/or to a reference naturally-occurring protein. In some embodiments, such stapled peptides are implemented in (e.g., expressed by and/or in) a biological system (e.g., a phage display library). In some embodiments, such stapled peptides are generated utilizing synthetic technologies. In some embodiments, peptides of the collection share a common length. In some embodiments, peptides of the collection share one or more particular sequence elements (e.g., residues of identity, number and/or relative spacing of cysteine residues, etc.). In some embodiments, amino acid residues at one or more positions, e.g., 1, 2, 3, 4, 5, 6, 7, 8, 9, 10 or more, are degenerate, e.g., among 2, 3, 4, 5, 6, 7, 8, 9, 10, 11, 12, 13, 14, 15, 16, 17, 18, 19, 20 or more, in many cases 10 or more, amino acid residues. In some embodiments, a collection comprises 10⁴, 10⁵, 10⁶, 10⁷, 10⁸ or more unique amino acid sequences. In some embodiments, a collection comprises 10⁴, 10⁵, 10⁶, 10⁷, 10⁸ or more unique stapled peptides.

In some embodiments, the present disclosure provides a collection of peptides. In some embodiments, a collection of peptides is a collection of stapled peptides. In some embodiments, peptides within a collection as described herein may share one or more structural features (e.g. length within a particular range; presence of particular sequence elements such as, for example, a sequence element found in a known interaction partner for a target of interest, or a set of amino acids that together support formation of a staple [e.g., two or more cysteine residues, positioned relative to one another so that a cysteine staple as described herein is or may be produced between a pair of them], presence of one or more staples which may, in some embodiments, be of the same type, etc., or any combination thereof, and in some embodiments, a particular collection may be characterized and/or defined by such shared structural feature(s)). In some embodiments, a common structure feature of peptides in a collection of peptides is or comprises two amino acid residues that can be stapled. In some embodiments, a common structure feature of peptides in a collection of peptides is or comprises a staple. In some embodiments, a common structural feature of peptides in a collection of peptides as described herein is or is at least two cysteine residues, positioned relative to one another so that a cysteine staple as described herein is or may be formed between a pair of them; in some embodiments, such peptides can be reacted with a compound of formula R-I, to produce a collection of cysteine stapled peptides. In some embodiments, as described herein, a collection of peptides, e.g., stapled peptides, comprises 1, 2, 3, 4, 5, 6, 7, 8, 9, or 10 more degenerate positions. In some embodiments, such degenerate positions generate a level of diversity, e.g., 10⁴, 10⁵, 10⁶, 10⁷, 10⁸ or more unique stapled peptides.

In some embodiments, a provided peptide collection is a collection of stapled peptides, each of which independently has an amino acid sequence that:
has a length within a range of a* and b*, where a* and b* are each integers independently selected from 2 through 100 and b* is greater than a*; and
includes at least one pair of residues are stapled (covalently linked with one another via a linker, as appreciated by those skilled in the art, other than through the peptide backbone).
   In some embodiments, a provided collection of peptides (or nucleic acids that encode them) is characterized in that peptides of the collection all include cysteine residues (e.g. a pair of cysteine residues), spaced relative to one another to permit cysteine stapling as described herein, but otherwise have independent amino acid sequences and, optionally, in that peptides of the collection all have the same length. In some such embodiments, the cysteine residues are located at corresponding positions in each of the peptides. In some embodiments, one or more (e.g., about or at least about 1, 2, 3, 4, 5, 6, 7, 8, 9, 10, 11, 12, 13, 14, 15, 16, 17, 18, 19, or 20, or about or at least about 5%, 10%, 20%, 25%, 30%, 40%, 50%, 60%, 70%, 80%, 90%, or 95%) or all positions other than the cysteine residues are degenerate (or randomized) within the collection (i.e., different peptides in the collection may have different residues at any of such one or more positions); in some such embodiments, one or more positions is fully degenerate within the collection in that every utilized amino acid (e.g., every natural amino acid) is included at the position in at least one peptide in the collection. In some embodiments, each position other than the cysteines is fully degenerate across the collection. In some embodiments, one or more positions are independently biased or enriched across the collection in that a single amino acid, or a plurality of different amino acids, each of which is selected from a restricted set (i.e., fewer than all possible amino acid residues and, in some embodiments, fewer than all natural amino acids) is present at each of such positions. In some embodiments, degeneracy and/or bias is introduced in one or more positions through genetic engineering and/or expression of selected nucleic acid sequences in a biological system. In some embodiments, degeneracy, randomization, enrichment or bias are introduced through chemical synthesis. In some embodiments, degree of degeneracy or bias at one or more positions in peptides of a peptide collection or library as described here is informed and/or selected by prior assessment of one or more binding characteristics of a related library or collection (e.g., with comparable cysteine residues). In some embodiments, such prior assessment is by high-throughput analysis (e.g., screening) of a collection or collections of cysteine stapled peptides against a target of interest and the use of high-throughput sequencing to decode the sequence of a subset of the collection of peptides can inform the production of a biased library.

In some embodiments, the present disclosure provides technologies for identification, characterization, and/or production of stapled peptide agents that bind a target of interest. In some embodiments, provided technologies involve contacting a target of interest with a collection of peptides (e.g., a collection of stapled peptides; in some embodiments, peptides of the collection share one or more structural features), and determining one or more characteristics (e.g., on-rate, off-rate, affinity [e.g., at one or more concentrations], specificity, binding curve, etc.) of an interaction between individual peptide(s) and the target. In some embodiments, such determining involves comparison with an appropriate reference (e.g., a positive or negative control, such as a previously known or characterized interaction between the target and a known interactor, or between different interacting partners).

In some embodiments, the present disclosure provides technologies for identification, characterization, production, and/or use of stapled peptide agents that can generate or enhance interactions of two targets of interest. In some embodiments, stapled peptide agents can bridge two targets of interests. In some embodiments, stapled peptide agents can enhance interactions of two targets of interest. In some embodiments, two targets of interest do not interact with each other absence of stapled peptide agents. In some embodiments, absence of provided stapled peptide agents, interactions of the two targets of interest are of low level or cannot be detected. In some embodiments, two targets of interest and a stapled peptide form a trimer (trimerize). In some embodiments, a stapled peptide forms a trimer with two targets of interest (such a stapled peptide may be referred to as a “trimerizer” or “molecular glue”).

In some embodiments, a target of interest may be or comprise a naturally-occurring epitope (e.g., that is or comprises a polypeptide, a glycan, etc.). In some embodiments, a target of interest is a natural polypeptide. In some embodiments, a target of interest is one for which a naturally-occurring interaction partner is not known. In some embodiments, a target of interest is one for which a naturally-occurring interaction partner is known (and, e.g., there is a desire to identify an alternative interaction partner—for example that may compete with a known interaction partner—and/or there is a desire to identify an alternative format of a known interaction partner).

In some embodiments, provided technologies may provide and/or utilize a phage expression system in which candidate peptides (e.g., whose amino acid sequences include at least two cysteine residues positioned relative to one another so as to support cysteine stapling as described herein). are produced as fusion proteins with a phage coat (e.g., protein pIII).

In some embodiments, the present disclosure provides technologies for developing (e.g., identifying and/or characterizing), producing, and/or using stapled peptide agents that bind to a target of interest, optionally with one or more predetermined characteristics of binding. In some such embodiments, one or more cysteine stapled peptides designed, produced, and/or characterized (e.g., via analysis utilizing phage display as described herein) is modified to substitute a cysteine staple with a non-cysteine staple. That is, in some embodiments, cysteine stapling technologies are utilized to identify and/or characterize peptides (i.e., peptide amino acid sequences and/or other structures) amenable to stapling to produce a stable structure with one or more desired binding attributes. Such peptides can be re-formatted in accordance with the present invention into corresponding peptides in which the cysteine staple has been replaced by a non-cysteine staple (e.g., through, optionally among other things, substitution of the cysteine residues involved in the staple(s) with other residues amendable to alternative stapling technologies (e.g., hydrocarbon stapling, heteroatom stapling, etc.).

In some embodiments, technologies provided by the present disclosure relate to peptides (e.g., individual peptides and/or peptide collections) having the structure of formula I:

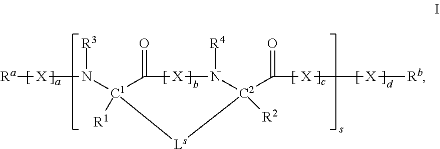

or a salt thereof, wherein:
each of R^{a}, R¹, R², R³, and R⁴ is independently R′;
R^{b} is R′, —OR′ or —N(R′)₂;
each X is independently an amino acid residue;
each of a, b, c, s, and d is independently an integer 1-20 inclusive;
each of C¹ and C² is independently a carbon atom;
L^{s} is -L^{s1}-S-L^{s2}-S-L^{s3}- or L;
L^{s1}, L^{s2} and L^{s3} are each independently L; each L is independently a covalent bond, or an optionally substituted, bivalent C₁-C₂₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L^{s} is -L^{s1}-S-L^{s2}-S-L^{s3}-.

In some embodiments, R^{a} is R′, wherein R′ is as described in the present disclosure. In some embodiments, R^{a} is —H. In some embodiments, R^{a} is R—C(O)—.

In some embodiments, the present disclosure provides a collection of stapled peptides, wherein each of the stapled peptides independently has the structure of formula I or a salt thereof.

In some embodiments, a is 1. In some embodiments, a is 2. In some embodiments, a is 3. In some embodiments, a is 4. In some embodiments, a is 5. In some embodiments, a is 6. In some embodiments, a is 7. In some embodiments, a is 8. In some embodiments, a is 9. In some embodiments, a is 10. In some embodiments, a is 11. In some embodiments, a is 12. In some embodiments, a is 13. In some embodiments, a is 14. In some embodiments, a is 15. In some embodiments, a is 16. In some embodiments, a is 17. In some embodiments, a is 18. In some embodiments, a is 19. In some embodiments, a is 20.

In some embodiments, R¹ is R′ as described in the present disclosure. In some embodiments, R¹ is R as described in the present disclosure. In some embodiments, R¹ is —H. In some embodiments, R′ is not H.

In some embodiments, R² is R′ as described in the present disclosure. In some embodiments, R² is R as described in the present disclosure. In some embodiments, R² is —H. In some embodiments, R² is not H.

In some embodiments, R³ is R′ as described in the present disclosure. In some embodiments, R³ is R as described in the present disclosure. In some embodiments, R³ is —H. In some embodiments, R³ is not H.

In some embodiments, R⁴ is R′ as described in the present disclosure. In some embodiments, R⁴ is R as described in the present disclosure. In some embodiments, R⁴ is —H. In some embodiments, R⁴ is not H.

In some embodiments, C¹ is achiral. In some embodiments, C¹ is chiral. In some embodiments, C¹ is (R). In some embodiments, C¹ is (S).

In some embodiments, C² is achiral. In some embodiments, C² is chiral. In some embodiments, C² is (R). In some embodiments, C² is (S).

In some embodiments, b is 2-11. In some embodiments, b is 2. In some embodiments, b is 3. In some embodiments, b is 4. In some embodiments, b is 5. In some embodiments, b is 6. In some embodiments, b is 7. In some embodiments, b is 8. In some embodiments, b is 9. In some embodiments, b is 10. In some embodiments, b is 11.

In some embodiments, c is 1. In some embodiments, c is 2. In some embodiments, c is 3. In some embodiments, c is 4. In some embodiments, c is 5. In some embodiments, c is 6. In some embodiments, c is 7. In some embodiments, c is 8. In some embodiments, c is 9. In some embodiments, c is 10. In some embodiments, c is 11. In some embodiments, c is 12. In some embodiments, c is 13. In some embodiments, c is 14. In some embodiments, c is 15. In some embodiments, c is 16. In some embodiments, c is 17. In some embodiments, c is 18. In some embodiments, c is 19. In some embodiments, cis 20.

In some embodiments, s is 1-5. In some embodiments, s is 1. In some embodiments, s is 2. In some embodiments, s is 3. In some embodiments, s is 4. In some embodiments, s is 5.

In some embodiments, d is 1. In some embodiments, d is 2. In some embodiments, d is 3. In some embodiments, d is 4. In some embodiments, d is 5. In some embodiments, d is 6. In some embodiments, d is 7. In some embodiments, d is 8. In some embodiments, d is 9. In some embodiments, d is 10. In some embodiments, d is 11. In some embodiments, d is 12. In some embodiments, d is 13. In some embodiments, d is 14. In some embodiments, d is 15. In some embodiments, d is 16. In some embodiments, d is 17. In some embodiments, d is 18. In some embodiments, d is 19. In some embodiments, d is 20.

In some embodiments, R^{b} is R′ as described in the present disclosure. In some embodiments, R^{b} is R as described in the present disclosure. In some embodiments, R^{b} is —H. In some embodiments, R^{b} is —OR′ wherein R′ is as described in the present disclosure. In some embodiments, R^{b} is —OH. In some embodiments, R^{b} is —N(R′)₂, wherein each R′ is independently as described in the present disclosure. In some embodiments, R^{b} is —NH(R′), wherein R′ is independently as described in the present disclosure. In some embodiments, R^{b} is —NH₂, wherein R′ is independently as described in the present disclosure.

In some embodiments, stapled peptides or collections thereof are prepared through chemical synthesis. For example, in some embodiments, degeneracy can be introduced independently at one or more positions through utilization of a mixture of amino acids.

### BRIEF DESCRIPTION OF THE DRAWINGS

FIG. 1. Certain useful technologies for designing, producing, and screening stapled peptides and collections thereof. High-throughput and parallelized phage screening identifies Helicons for multiple protein targets in a single experiment. (A) Cysteine-stapled Helicons are displayed as N-terminal fusions to the pIII protein on M13 bacteriophage. Randomized residues are shown as x. A short glycine-rich linker (not shown) connects the C-terminus of the displayed peptide to the phage particle. The example shown has the sequence of SEQ ID NO: 870. (B) Many phage screens (e.g., 48 or 96) can be run in parallel in multi-well plates. In some embodiments, each well receives approximately 100 copies of each of the 10⁸ library members (10¹⁰ phage total), allowing direct comparison between all screens. Following binding to target on beads and subsequent washes, phage DNA is directly extracted, amplified and sequenced. A “spike-in” sequence of known quantity is added to enable the normalization of sequencing reads and quantitative comparison across samples. (C) A scheme for a screen. Multiple proteins are screened, each under multiple concentrations (top), along with blank beads (not shown). Other controls and comparisons, such as proteins bound to known ligands or proteins with different post-translational modifications, can be included to enable targeted searches for Helicons with the desired binding mode or mechanism (middle). Following hit selection, sequences are clustered into families, represented here as sequence logos (bottom). The examples shown have the sequences of SEQ ID NO: 871-875, respectively, in order of appearance.

FIG. 2. Stapled peptides binding known binding motifs, variants of known motifs, and new alpha-helix binding sites. (A) Four beta-catenin binding clusters that were observed to compete with an Axin peptide in the phage screen. Clusters C31 and C32 appear to be “shifts”, as are C33 and C34, and these two pairs are, in turn, “flips” of each other. Figure discloses SEQ ID NO: 876. (B) Representative Helicons from C31-34 bind to β-catenin in vitro and compete with β-catenin-Axin binding, consistent with phage screen results. Figure discloses SEQ ID NOS 904-905, 912, 901-903, 877, 907, 155, 908-909, 180, 910-911, respectively, in order of appearance. (C) Overlay of X-ray co-crystal structures of FP01567 (C33), FP05874 (C35), and the β-catenin-binding domain of Axin (62). FP01567 binds at the same site as Axin (62), while FP05874 binds closer to the C-terminus of the protein. (D) FP01567 engages the β-catenin surface with similar side-chain contacts as Axin, but with an opposite N-to-C orientation of the alpha-helix. Key logo residues are marked with dots and indicated as letters. (E) Conserved residues in the C35 cluster logo, marked with dots and indicated with letters, correspond to key binding contacts between FP05874 and β-catenin. The N-to-C orientation of the helix is indicated.

FIG. 3. Certain beta-catenin binding stapled peptides, structures and data. (A) Two opposite faces of the β-catenin ARM domain structure (lilac, PDB 1QZ7) are shown overlaid with several of its binding partners, as determined by x-ray crystallography for BCL9 (1), TCF4 (2), Axin (3), and ICAT (4). (B) Representative surface plasmon resonance (SPR) analysis of the interaction of Helicons with immobilized b-catenin (K_{D} values and SD based on triplicates). The responses observed in sensorgrams for each concentration of Helicon were plotted to determine the binding constant (K_{D}). (C) SPR analysis of cluster C33 Helicon FP01567 and an alanine-scanning series across the non-crosslinked residues of the peptide demonstrates the importance of the conserved logo residues for binding (n=2 or 3; data are presented as mean±SD). n.d., no data. Figure discloses SEQ ID NOS 877-887, respectively, in order of appearance.

FIG. 4. Stapled peptides binding the PUB and UBA domains. (A) Five RNF31 PUB binding clusters, which can be grouped into two families. Shifts are observed within both families. (B) Overlay of x-ray co-crystal structures of a member from each family of clusters, FP06649 (C44) and FP06652 (C41) (left). Key logo residues are marked with dots and indicated as letters (right). Despite binding the same α-helix recognition site in the same N-to-C orientation, the two Helicons engage the protein surface with different sidechains. (C) Three RNF31 UBA binding clusters, which can be grouped into two families. C46 and C47 appear to be shifts of each other. (D) Surface plasmon resonance (SPR, Biacore) binding of FP06655 (C46) to the RNF31 UBA domain. (E) X-ray co-crystal structure of FP06655 bound to the RNF31 UBA domain. A significant conformational rearrangement is observed (lilac overlay) relative to the previously reported co-crystal structure of the RNF31 UBA domain (grey) with its binding partner Sharpin/SIPL1 (peach).

FIG. 5. Certain RNF31 binding stapled peptides, structures and data. (A) Cluster C44 Helicon FP06649 and Cluster C41 Helicon FP06652 share an RNF31 UBL-binding site, but bind with different orientations compared to Otulin, as shown by x-ray crystallography. (B) Helicons FP06649 and FP06652 compete with fluorescently labeled Otulin peptide for binding to RNF31-PUB as monitored by fluorescence polarization, while the RNF31 UBA-binding helicon, FP06655 does not compete (n=2; data are presented as mean±SD), and by Surface Plasmon Resonance (SPR, Biacore). RNF31 UBA-binding Helicon FP06655 from Cluster C36 does not compete for binding to the Otulin site on RNF31 PUB. (C) Helicon FP06655 and unlabeled Sharpin/SIPL1 UBL domain compete with fluorescently labeled 5FAM-FP06655 for binding to RNF31-UBA, while the RNF31-PUB binding peptides (FP06649 and FP06652) do not compete (n=3; data are presented as mean±SD).

FIG. 6. Certain stapled peptides binding CDK2 or PPIA and data. (A) Phage cluster logos for two CDK2 binding clusters, and x-ray co-crystal structures of one member from each, FP19711 (C51) and FP24322 (C52). These two Helicons bind distinct sites on the CDK2 surface. (B) FP19711 shows selectivity for CDK2 and the CDK2-CCNE1 complex over CDK1 or the CDK1-CCNA2 complex on phage. (n=2; data are presented as mean±SD) (C) The FP19711 binding site on CDK2 contains several amino acid differences relative to CDK1. (D) Two related clusters that bind PPIA (C54 and C53 with their logos shown), and x-ray co-crystal structures of one member from each: FP29103 (C54), discovered from a truncated version of our standard phage library) and FP29092 (C53). These Helicons recognize a similar site as natural product CsA (left). (E) Helicon binding overlaps with the PPIA substrate-binding site (example from the HIV capsid protein shown). Key logo residues are marked with dots and indicated as letters. (F) PPIA-binding Helicons inhibit peptidyl-prolyl cis-trans isomerase activity, similar to the control CsA but with much higher IC₅₀ (n=3; data are presented as mean±SD).

FIG. 7. Certain CDK2- and PPIA-binding stapled peptides, structures and data. (A) Cluster C51 peptide FP19711 and Cluster C52 peptide FP24322 bind distinct sites from the orthosteric CDK2 ATP site, and FP19711 induces a rotation of the N-terminal lobe. (B) The FP19711-CDK2 co-structure indicates that the N- and C-terminal-most residues of the peptide do not interact with CDK2. Truncation of FP19711 to FP33215 improves its CDK2-binding affinity to −300 nM. Figure discloses SEQ ID NOS 888-889, respectively, in order of appearance. (C) By SPR (Biacore), FP19711 and FP24322 bind CDK2. FP19711 and its truncated version, FP33215, also bind the active CDK2 (with T160 phosphorylated) and active CDK2: Cyclin E1 (CCNE1) complex. In addition, FP19711 retains its CDK2-binding affinity in the presence of ATP-analog (AMPPNP) or ATP-competitive inhibitors (Dinaciclib and Zotiraciclib), suggesting it is not ATP-competitive. (D) Compared with ATP-competitive CDK2 inhibitors, FP19711 does not compete with fluorescently labeled ATP analog (BODIPY-ATP-yS) for binding CDK2 as assessed by fluorescence polarization. This is consistent with its binding to an allosteric site on CDK2. (E) By SPR (Biacore), Cluster C53 peptide FP29092 and Cluster C54 peptide FP29103 bind to PPIA. (F) Co-crystal structure of Cluster C54 peptide FP29102 Helicon with PPIA shows that it binds a site similar to that of Cyclosporine A and peptide substrates. (G) Helicons FP29092 and FP29103 compete with Cyclosporine A for binding to PPIA as monitored by Surface Plasmon Resonance (SPR, Biacore).

FIG. 8. Certain PD-L1-binding stapled peptides, PD-L1:PD-1 interaction inhibition and of PD-L1 dimerization. (A) Phage cluster logos for two PD-L1 binding clusters, and x-ray co-crystal structures of one member from each, FP28132 (C61) and FP30790 (C62). Both Helicons recognize sites that overlap with the PD-1 binding site on PD-L1. (B) The FP28132-PD-L1 complex appears to dimerize in the crystal lattice, in a manner distinct from the previously reported PD-L1 dimerizer BMS-1001. (C) FP28132 and BMS-1001, but not the closely matched FP28132-derived control FP28141, inhibits PD-L1:PD-1 binding in a competition ELISA assay (n=2; data are presented as mean±SD) and (D) induce PD-L1 dimerization in solution as assayed by TR-FRET (n=3 for FP peptides and n=2 for BMS-1001; data are presented as mean±SD).

FIG. 9. Certain PD-L1 binding stapled peptides, structures and data. (A) SPR binding and competition assays show that Cluster C62 peptide FP30790 binds to PD-L1 (left) and while it competes for binding to PD-Li with PD-Li receptor PD-1, Cluster C61 Helicon FP28312 and the small-molecule inhibitor of the PD-1/PD-L1 interaction, BMS-1001 compete more potently (right). FP28141 is a point mutant of FP28132 with an alanine residue replacing a conserved tryptophan from the phage cluster logo, that does not bind PD-Li (see S6B). (B) Surface plasmon resonance (SPR, Biacore) shows binding of FP28132 and additional Cluster C61 Helicons FP28135 and FP28136 to PD-Li, while FP28141 does not bind. Figure discloses SEQ ID NOS 913-914, and 890-891, respectively, in order of appearance. (C) Co-crystal structure of FP28135 and FP28136 with PD-Li shows a symmetric dimer of two Helicon/PD-L1 complexes. (D) Close examination of the co-structure reveals an extensive series of contacts between both the two FP28132 protomers and the two PD-L1 protomers. (E) Analytical SEC suggests that the apo-PD-Li and FP30790-PD-L1 are monomeric, while FP28132-PD-L1 complex is a dimer in solution.

FIG. 10. Certain trimerizer technologies and data as examples. (A): A scheme illustrating a useful process. (B) Direct fluorescence polarization (labeled MDM2). (C) Surface plasmon resonance (ABA mode, immobilized MDM2).

FIG. 11. X-ray co-crystal structure of the beta-catenin-MDM2-Helicon Complex (A) and fluorescence polarization binding data (B) confirm ternary complex formation at nanomolar concentrations. Ternary complex formation is inhibited in the presence of MDM2-binding peptide.

FIG. 12. Certain griptide technologies and data as examples. (A): A scheme illustrating a useful process. (B): High affinity beta-catenin griptides and selectivity profiles. Biochemical validation of certain griptides for beta-catenin: Griptide disulfide-dependent conformation populations under non-reduced (C) and reduced (D) conditions; high affinity griptides for beta-catenin observed using SPR (E).

FIG. 13. Agents and binding sites for members of HECT and CRL E3 ligase families. (A) An example prototypical HECT E3 ligase where two lobes of the −40 kDa HECT domain are flexibly tethered. A E2-binding surface is on the larger N-lobe, while the active-site cysteine is on the C-lobe. Ubiquitin (Ub)-loaded E2 brings the HECT and E2 cysteines into close proximity for ultimate Ub transfer to protein targets bound to the substrate-binding domain (SBD) of the E3 ligase. Certain representative clusters discovered by screening a Helicon library against WWP-family protein constructs that either stabilize the autoinhibited, inactive state (WWP1^{WW-HECT}) or that can adopt both active and inactive conformations (WWP1^{HECT}, WWP2^{HECT}) are depicted as logos, with grey indicating residues that were fixed for all library members. (B) Co-crystal structures of Helicon H302 with WWP1^{HECT} and (C) overlaid structures of H301, H308, and H305 with WWP2^{HECT}, solved at 2.43-3.60 Å resolution. (D) An example prototypical multi-subunit Cullin-RING (CRL) E3 ligase with an E2-binding RING domain, shown with a single adaptor subunit (as in CUL3-scaffolded CRLs). The adaptor component of CUL1-, CUL2-, CUL4-, CUL5-, and CUL7-scaffolded CRLs can comprise additionally a receptor subunit (e.g. VHL plus ELOBC for CUL2). Certain representative clusters derived from screening a Helicon library against CUL5, VHL-ELOBC, CUL1, and FBXW7-SKPI are depicted as logos as in A. (E) Co-crystal structures of H314 bound to the N-terminal domain of CUL5 (CUL5^{NTD}) and (F) H313 binding to VHL in the VHL-ELOBC complex.

FIG. 14. Provided technologies can bind HECT E3 family. Helicon binding and biochemical activity against WWP members of the HECT E3 family. (A) Heatmap of E3 ligase gene expression across adult normal tissues using RNA-Seq data from the GTEx Portal (gtexportal.org). Colors represent the median expression in transcripts per million (TPM) in indicated tissue. (B) An example prototypical RBR E3 ligase highlighting the two RING domains of this family. (C) SPR validation of certain hits derived from screening against three HECT-containing proteins defines four groups of Helicons based on their binding to both inactive and active forms of both HECT domains (Group-1), to both isolated WWP1 and WWP2 HECT domains (Group-2), to the WWP2 HECT domain (WWP2^{HECT}) only (Group-3), or to the WW domain only (Group-4) (n=2). ^{#}The Group-2 logo shown (red) represents Cluster C73 (C73), but the C71 logo (including from H304) is similar. Figure discloses SEQ ID NOS 501, 503, 512, 530, 532, 892, 516-517, 520, 545, and 556-557, respectively, in order of appearance. (D) Helicons from Groups-1 through -3 could partially inhibit the in vitro auto-ubiquitylation activity of WWP2^{HECT} (n=3; data are presented as mean±SD). (E) X-ray co-crystal structures of Helicons from Groups-1 through -3 with WWP2^{HECT} as in FIG. 13, (A). (F) The structural overlay shown in FIG. 13, with the addition of the WWP2 helix residues 382-395 that form the inhibitory linker domain of WWP2 (aqua) and the E2 UBCH7 (PDB: 5HPT) at the Helicon H301-binding interface with WWP2^{HECT}.

FIG. 15. Provided technologies can bind CRL family E3 ligases. Helicon binding to CRL family E3 ligases. (A) SPR analysis of Helicon H313 revealing its specificity for VHL-ELOBC (left) over SOCS2-ELOBC (right) (n=3). (B) A competition FP assay was performed using VHL-ELOBC and VHL-binding fluorescent probe HXC78 revealing that Helicon H313 does not compete with HXC78 for binding VHL-ELOBC, while VH298—a molecule related to HXC78—potently competes (n=3; data are presented as mean±SD). VH298 binds VHL with a K_{D} of 90 nM. (C) SPR analysis of H314 binding to CUL5 (left) and a competition SPR (ABA) assay (right) reveal that H314 binds to CUL5 to disrupt its interaction with SOCS2-ELOBC. (n=3) (D) H314 overlaid on the full-length CUL5 bound to RING-box protein 2 (CUL5-RBX2, PDB 6V9I) using PyMol (left). The surface of CUL5 and RBX2 are shown in grey and yellow, respectively. Helicon H313 was overlaid on the CUL2-RBX1-ELOBC-VHL E3 ligase complex (PDB 5N4W) using PyMol, with CUL2 and RBX1 shown in grey and yellow, respectively. The small molecule VHL ligand, VH298, binds on the opposite side from Helicon H313 (PDB 5LLI) (right). (E) Helicon H316 was crystallized with the CUL4B N-terminal domain (CUL4B^{NTD}) that also harbors V516R/L520D point mutations near the truncation site to assist with protein expression—as reported for CUL1 (Zheng, N. et al. Structure of the Cul1-Rbx1-Skp1-F boxSkp2 SCF ubiquitin ligase complex. Nature 416, 703-709 (2002))—and the co-structure was solved at 2.89 Å resolution (left). The corresponding Helicon H316-binding site on CUL4A, which shares an 81% sequence identity with CUL4B, is located at the interface between CUL4A^{NTD} and the C-terminal domain (CUL4A^{CTD}), indicating a non-physiological Helicon interaction (right). The overlaid CUL4A^{NTD} structure is from PDB 2HYE.

FIG. 16. Provided technologies can bind to members of the RING/U-Box E3 ligase family. (A) An example prototypical RING/U-Box E3 ligase comprising a single RING domain and a direct E2-to-substrate catalytic mechanism. The U-Box domain is at the C-terminus and serves as the binding site for the ubiquitin-charged E2 ligase and acts to promote ubiquitin transfer. (B) Representative clusters and their logos derived from screening a Helicon library for binders to MDM2 and CHIP. (C) Helicon H318 was crystallized with the TPR domain of CHIP (residues 21-154, CHIP^{TPR}) and the co-structure was solved at 1.47 Å resolution.

FIG. 17. Identification of Helicon residues responsible for binding to MDM4 and CHIP. (A) An additional H317-CHIP co-structure view. Helicon H317 was crystallized with the TPR domain of CHIP (residues 23-154, CHIP^{TPR}) and the co-structure was solved at 2.06 Å resolution. (B) Published structure of ATSP-7041 with MDM4 (referred to here as P320, PDB 4N5T). (C) Comparison of the H317-CHIP^{TPR}(residues 21-154) structure and the 1.47 Å resolution structure of H318. The two Helicons are from the same CHIP-binding cluster, including a shared Glu residue. E9 of H317 directly interacts with CHIP whereas E10 of H318 does not, suggesting the regional plasticity of the interaction between Helicon and protein in some instances.

FIG. 18. Provided technologies can modulate polypeptide interactions. Illustrated is an example of a general method for the discovery of trimerizer Helicons that induce a novel interaction between two target proteins. (A) In the first of two high-throughput screens (Screen 1), Helicons that bind to the presenter E3 proteins (green) are identified from a naive 10⁸-member phage display library. Screen 2 begins by designing and generating a focused Helicon library based on hits from Screen 1. Helicon residues involved in E3 binding are determined by the prominence of the amino acid letters in the cluster logo, and from Helicon co-structures with the E3 if available. These residues are fixed and the remaining residues are randomized in focused library primers that are used to build a library of 20-mer Helicons with a diversity of -10′ members. Screening for target binders from the diversified library is done in the presence or absence of the E3 presenter protein to identify trimerizer hits that bind the target in a presenter-dependent manner. (B) Two examples of focused library designs. In the case of CHIP cluster C84, residues 10 and 14 are fixed, while the remaining residues are either partially randomized (residues 9, 12, 13) since they were defined by two to five favored residues in the logo, or fully randomized (residues 1-3, 5, 6, plus six added residues flanking the 14-mer core). Amplification primers contain partially degenerate codons (X) or semi-degenerate codons (e.g. RTT for valine or isoleucine at position 13 in the CHIP focused library) for library production. The four fixed cysteine and alanine residues from the naive library design may remain fixed in the focused library. Similar design logic is used for the MDM2 cluster C85. Figure discloses SEQ ID NOS 893-896, respectively, in order of appearance. (C) Hits selected from focused phage libraries were screened for binding to the target (blue) in the presence or absence of presenter (green). Quantitation of target binding (illustrative tabular values) is used to identify trimerizer Helicons that bind the target only in the presence of the presenter (cooperatively), and these are clustered based on sequence (top). Helicons that bind the target even in the absence of presenter (non-cooperatively) are ignored (bottom). Figure discloses SEQ ID NOS 897, 898-900, and 499, respectively, in order of appearance.

FIG. 19. Provided technologies can modulate polypeptide interactions. Illustrated are certain trimerizer Helicons that induce the interaction between CHIP and TEAD4 or PPIA. (A) Trimerizer clusters and ternary complex formation for Helicons that bind TEAD4 in a CHIP-dependent manner (CHIP-TEAD4) and those that bind PPIA in a CHIP-dependent manner (CHIP-PPIA). Two clusters are shown for CHIP-TEAD4 and four clusters are shown for CHIP-PPIA. For TR-FRET, Helicons H321 and H322 from C87 were added at increasing concentrations to biotinylated TEAD4 and Alexa Fluor 488-labeled CHIP, and FRET signal indicating CHIP-TEAD4 binding was monitored upon addition of terbium (III)-labeled streptavidin. The chimeric (heterobifunctional) peptide control P325 comprises CHIP- and TEAD4-interacting peptides connected by a linker, and displays the expected “hook” effect (Paiva, S.-L. & Crews, C. M. Targeted protein degradation: elements of PROTAC design. Curr Opin Chem Biol 50, 111-119 (2019)). Similarly, CHIP-PPIA ternary complex formation was evaluated with TR-FRET between biotinylated PPIA and Alexa Fluor 488-labeled CHIP to characterize H326 from C88 and H327 and H328 from C89. Ternary complex formation induced by H326 occurs between the cyclosporin A (CsA) binding site of PPIA and the H318/substrate-binding site of CHIP, and CsA and H318 could compete with ternary complex formation as expected. (B) SPR assays in ABA mode with immobilized CHIP²³³⁰³ show H321 and H322-dependent binding of TEAD4 to CHIP. (C) Helicons were added at various concentrations to TEAD4 and a fluorescent 44-residue YAP peptide for Competition FP assays. H321 could only interfere with the YAP1-TEAD4 interaction in the presence of CHIP. An unlabeled YAP1 fragment (residues 50-100) was included as a positive control.

FIG. 20. Provided technologies can modulate polypeptide interactions. Illustrated are certain biochemical characterization of CHIP and MDM2 trimerizer Helicons. (A) TR-FRET was performed to assess H323- and H324-mediated ternary complex formation between CHIP and TEAD4. Both Helicons are from C87. (B) TEAD4 competition FP was performed to demonstrate inhibition of the TEAD4-YAP1 interaction by H322 in the presence of CHIP with additional positive and negative controls (left). Competition FP with CHIP shows that H321 and a C-terminally truncated version of H321 (H322) bind to CHIP alone but with a higher EC₅₀ than the CHIP-binder H318. The chimeric P325 also binds to CHIP with a higher EC₅₀. (C) Direct FP was performed to assess ternary complex formation mediated by MDM2-O-catenin trimerizers H329, H333, and an N-terminally truncated version of H329, H331, using the β-catenin Armadillo domain (top left). Similarly, direct FP assays reveal ternary complex formation between the p53-binding domain of MDM2 and full-length β-catenin mediated by H330 (top middle). SPR (ABA mode) assay using immobilized MDM2 to demonstrate the trimerizer activity of H330 (top right). Additional characterization of the Helicons via MDM2-competition FP. Control peptides, ATSP-7041 and H134 interact with MDM2. Meanwhile, both H330 and H332 alone only interact with the MDM2 p53-binding domain with a low affinity (IC₅₀>10 μM, bottom left). SPR assay performed to assess the interaction of Helicons with immobilized β-catenin shows that H330 weakly interacts with β-catenin in the absence of MDM2. In contrast, H332 shows no detectable binding under these conditions (bottom middle and right). (D) The direct FP assay for ternary complex formation shows that H330 competes with β-catenin-binder ICAT and MDM2-binder ATSP-7041, confirming H330-binding sites on both MDM2 and β-catenin.

FIG. 21. Provided technologies can modulate polypeptide interactions. Illustrated are certain trimerizer Helicons that induce the interaction between between MDM2 and β-catenin. (A) Three trimerizer clusters that cooperatively induce the interaction between MDM2 and β-catenin are presented as examples. (B) Ternary complex formation was assessed using direct FP, where Helicon and β-catenin are incubated with fluorescent p53-binding domains of MDM2 or MDM4 over a range of Helicon concentrations. Helicons H330, H332, and H334 from trimerizer clusters C91, C92, and C93, respectively, as well as the chimeric (heterobifunctional) peptide P335 comprising linked MDM2- and β-catenin-interacting peptides could promote ternary complex formation (left). Ternary complex formation was dependent on MDM2 and did not occur with H330, H332, or H334 when using the MDM2 homolog MDM4, which shares -55% sequence identity with MDM2 in the p53-binding domain (right). (C) SPR was performed by immobilizing the Armadillo domain of β-catenin and flowing through the p53-binding domain of MDM2, in the presence of DMSO vehicle (left), 1 μM H330 (middle), or 1 μM H332 (right). Concentrations of MDM2 from bottom to top in the sensorgram are 2-fold dilutions from 625.0 nM to 9.8 nM (n=3).

FIG. 22. Provided technologies can promote polypeptide interactions. In some embodiments, trimerizer Helicons promote cooperative interactions between MDM2 and β-catenin. (A) X-ray co-crystal structures of H330 and H332 in complex with MDM2 and β-catenin. Below are close-up views of the (B) H330- and (C) H332-mediated ternary complexes formed with MDM2 and β-catenin, revealing specific residue-level molecular recognition events that drive cooperative formation of complexes.

FIG. 23. Structural characterization of certain Helicon:MDM2:β-catenin ternary complexes. (A) X-ray co-crystal structures of the complexes between β-catenin and MDM2 mediated by two additional Helicons, H329 from C91 and H333 from C92. (B) Both Helicon and MDM2 surfaces contribute to binding to β-catenin and thus ternary complex formation. The Helicon- and MDM2-adjacent surfaces (within 4.5 Å distance) of β-catenin are highlighted in teal and green, respectively, and orange for surfaces adjacent to both Helicon and MDM2. Electron density maps of Helicons (2mFo-dFc, 1.0 s) and isolated MDM2-Helicon interactions.

### DETAILED DESCRIPTION OF CERTAIN EMBODIMENTS

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is a stapled peptide.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation, or combinations thereof. Unless otherwise specified, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof.

Alkenyl: As used herein, the term “alkenyl” refers to an aliphatic group, as defined herein, having one or more double bonds.

Alkenylene: The term “alkenylene” refers to a bivalent alkenyl group.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkylene: The term “alkylene” refers to a bivalent alkyl group.

Amino acid: In its broadest sense, as used herein, refers to any compound and/or substance that can be incorporated into a polypeptide chain, e.g., through formation of one or more peptide bonds. In some embodiments, an amino acid comprising an amino group and an a carboxylic acid group. In some embodiments, an amino acid has the structure of NH(R^{a1})-L^{a1}-C(R^{a2})(R^{a3})-L^{a2}-COOH, wherein each variable is independently as described in the present disclosure. In some embodiments, an amino acid has the general structure NH(R′)—C(R′)₂—COOH, wherein each R′ is independently as described in the present disclosure. In some embodiments, an amino acid has the general structure H₂N—C(R′)₂—COOH, wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%,9%,8%,7%,6%,5%,4%,3%,2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, etc..) is considered to be associated with a particular disease, disorder, or condition, if its presence, level and/or form correlates with incidence of and/or susceptibility to the disease, disorder, or condition (e.g., across a relevant population).

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR* (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; RingeR's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds (e.g., phosphate linkage groups of oligonucleotides, phosphorothioate linkage groups of oligonucleotides, etc.) with bases. Representative alkali or alkaline earth metal salts include salts of sodium, lithium, potassium, calcium, magnesium, and the like. In some embodiments, pharmaceutically acceptable salts are ammonium salts (e.g., —N(R)₃/). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protecting Group: The phrase “protecting group,” as used herein, refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, propionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In some embodiments, example substituents are described below.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₁₀ (e.g., C₁₋₉, C₁₋₆, C₁₋₅, C₁₋₄, etc.) aliphatic, C₁₋₁₀ (e.g., C₁₋₉, C₁₋₆, C₁₋₅, C₁₋₄, etc.) heteroaliphatic having 1-5 heteroatoms independently selected from nitrogen, oxygen, sulfur, silicon and phosphorus, —CH₂—(C₆₋₁₄ (e.g., C₆₋₁₀, C₆, etc.) aryl), —O(CH₂)₀₋₁(C₆₋₁₄ (e.g., C₆₋₁₀, C₆, etc.) aryl), —CH₂-(5-14 (e.g., 5-10, 5-6, 5, 6, 9, 10, 14, etc.) membered heteroaryl ring having 1-5 heteroatoms independently selected from nitrogen, oxygen, and sulfur), a 3-10 (e.g., 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 3-10 (e.g., 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3-6 (e.g., 4-6, 5-6, etc.) membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 3-6 (e.g., 4-6, 5-6, etc.) membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 4-6, 5-6, etc.) membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12 (e.g., 3-10, 3-7, 3-6, 5-10, 5-7, 5-6, etc.) membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

Subject: As used herein, the term “subject” or “test subject” refers to any organism to which a provided compound or composition is administered in accordance with the present disclosure, e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Unless otherwise specified, salts, such as pharmaceutically acceptable acid or base addition salts, stereoisomeric forms, and tautomeric forms, of provided compound are included. As appreciated by those skilled in the art, agents, compounds, etc. may be provided and/or utilized as various forms including various pharmaceutically acceptable salt forms, solvate forms, etc.

Unless otherwise clear from context, in the present disclosure, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

### Peptide Agents

Technologies provided by and/or described in the present disclosure particularly relate to peptide agents, e.g., to agents that are or comprise stapled peptides.

Among other things, the present disclosure provides technologies for developing, identifying, characterizing, and/or making stapled peptides that can modulate one or more functions of a target of interest. In some embodiments, stapled peptides are useful for treating various conditions, disorders, and/or diseases that are associated with the target of interest. Exemplary structural elements of provided stapled peptides are described herein.

Among other things, as discussed herein, the present disclosure encompasses the insight that biological systems can be utilized to generate peptide agents that include cysteine residues appropriate for and/or amenable to cysteine stapling. The present disclosure further appreciates that use of such biological systems can permit high-throughput production and/or assessment of cysteine stapled peptides (e.g., with respect to one or more (e.g., specificity, affinity, on-rate, off-rate, stability to competition, binding curve over a range of conditions such as concentration, temperature, pH, osmolality, presence or amount of competitor, etc.) characteristics of their binding interaction(s) with one or more targets of interest.

Thus, in some embodiments, the present disclosure provides peptide agents that include two or more cysteine residues, spaced apart from one another appropriately so as to support cysteine stapling. In some embodiments, provided are collections of such peptide agents. In some embodiments, provided are cysteine stapled peptides and/or collections thereof.

The present disclosure further appreciates that insights gleaned from producing, screening and/or otherwise analyzing or characterizing one or more cysteine stapled peptides can inform design, production, and/or use of analogous or comparable (e.g., containing the same or substantially the same [e.g., but for one or more conservative substitutions and/or one or a small number of other changes] amino acid sequence except for substitution of those cysteine residue(s) that participate in a staple with non-cysteine residue(s) that can or do participate in an analogous or comparable staple) peptide agents that share with their “parent” cysteine stapled peptide one or more binding characteristics with a particular target of interest. The present disclosure therefore provides peptide agents (e.g., that are or comprise stapled peptides) that correspond to cysteine stapled peptides (but include non-cysteine residue(s) rather than cysteines that participate in the staple).

In some embodiments, peptides are stapled through non-cysteine residues.

In some embodiments, peptide agents are prepared utilizing chemical synthesis technologies. Various technologies for preparing peptides, e.g., solid phase synthesis, may be utilized in accordance with the present disclosure.

Certain useful technologies for preparing, identifying, characterizing, and using peptide agents and/or collections thereof, including peptide synthesis, staples, etc., are described in U.S. Ser. No. 11/198,713, US 20210179665, WO 2021119537, WO 2021188659, WO 2022020651, or WO 2022020652, the entirety of each of which is incorporated herein by reference.

### Amino Acid Sequence

One particular advantage of technologies provided by the present disclosure is that they permit discovery of and/or define amino acid sequences that are particularly useful for stapled peptides (e.g., that bind to a particular target of interest).

Thus, in some embodiments, the present disclosure provides amino acid sequences for stapled peptides. In some embodiments, stapled peptides comprising provided amino acid sequences interact with (e.g., directly bind to) a target of interest, and, in some embodiments, such binding displays one or more characteristics as discussed herein.

As will be appreciated by those skilled in the art reading the present disclosure, in some embodiments, the present disclosure defines useful amino acid sequences from a collection that may be highly diverse—e.g., that may include two or more amino acid alternatives at any one or collection (including all) of positions along the amino acid chain, except for those that participate in a staple which, in many embodiments, are cysteines.

In some embodiments, amino acid sequences utilized in peptide agents as described herein may be or comprises, or be derived from, a sequence that is found in nature or in an otherwise appropriate reference polypeptide (e.g., one that may be known to bind to a relevant target of interest, for example via an interaction characterized by one or more features as described herein.

In some embodiments, an amino acid sequence that is utilized in a peptide agent, or in a collection of peptide agents, as described herein, is a variant of a reference sequence in that (1) it includes a pair of cysteine residues, at least one or which is not found at a corresponding position in the reference sequence, that are amenable to or participate in a cysteine staple; and/or (2) it includes an amino acid substitution at one or more positions of the reference sequence. In some embodiments, a substitution may be a conservative substitution, as understood in the art. In some embodiments, a substitution may involve substitution of a homolog. In some embodiments, a homolog of an amino acid is a naturally occurring or non-naturally occurring amino acid that has one or more similar properties to the amino acid and or amino acid side-chains being replaced, for example, that is typically classified as similar to one another as “non-polar”, “polar”, “hydrophobic”, “hydrophilic”, “basic”, “acidic”, “aliphatic”, “aromatic”, and/or “similar size”.

For example, in some embodiments, depending on context, a homolog of leucine can be an optionally substituted amino acid selected from isoleucine, alanine, homoleucine, 3-cyclobutylalanine, alpha-neopentylglycine, 3-cyclopropylalanine, alpha-methylleucine, and 3-cyclohexylalanine; a homolog of isoleucine can be an optionally substituted amino acid selected from alanine, leucine, homoleucine, 3-cyclobutylalanine, alpha-neopentylglycine, 3-cyclopropylalanine, L-alloisoleucine, and alpha-methylleucine; a homolog of phenylalanine can be an optionally substituted amino acid residue selected from tryptophan, tyrosine, 3-(1-naphthylalanine), 3-(2-naphthylalanine), 2-chlorophenyalanine, 3-chlorophenylalanine, 4-chlorophenylalanine, 4-tert-butylphenylalanine, O-methyl tyrosine, homophenylalanine, 4-fluorophenylalanine, 4-methylphenylalanine, 4-bromophenylalanine, 4-phenyl-L-phenylalanine, 5-chlorotryptophan, 5-hydroxytryptophan, 4-trifluoromethylphenylalanine, 4-guanidino-L-phenylalanine, 2-quinolyl-L-alanine, 3-cyclobutylalanine, alpha-neopentylglycine, and L-2-aminoadipic acid; etc.

In some embodiments, a homolog of a non-polar amino acid is another non-polar amino acid. In some embodiments, a homolog of an amino acid comprising a non-polar side chain is another non-polar amino acid comprising a non-polar side chain.

In some embodiments, a homolog of a polar amino acid is another polar amino acid. In some embodiments, a homolog of an amino acid comprising a polar side chain is another polar amino acid comprising a polar side chain. In some embodiments, side chain of a polar amino acid is not charged at about pH 7.4. In some embodiments, side chain of a polar amino acid does not contain a basic or acidic group. In some embodiments, side chain of a polar amino acid comprises —OH. In some embodiments, side chain of a polar amino acid comprises an amide group.

In some embodiments, a homolog of a hydrophobic amino acid is another hydrophobic amino acid. In some embodiments, a homolog of an amino acid comprising a hydrophobic side chain is another amino acid comprising a hydrophobic side chain. In some embodiments, a hydrophobic side chain is optionally substituted C₁₋₆ aliphatic, wherein each substituent, if any, is a non-polar group. In some embodiments, a hydrophobic side chain is C₁₋₆ aliphatic. In some embodiments, a hydrophobic side chain is C₁₋₆ haloaliphatic. In some embodiments, a hydrophobic side chain is C₁₋₆ alkyl. In some embodiments, a hydrophobic side chain is C₁₋₆ haloalkyl.

In some embodiments, a homolog of a hydrophilic amino acid is another hydrophilic amino acid. In some embodiments, a homolog of an amino acid comprising a hydrophilic side chain is another hydrophilic amino acid comprising a hydrophilic side chain.

In some embodiments, a homolog of a basic amino acid is another basic amino acid. In some embodiments, a homolog of an amino acid comprising a basic side chain is another basic amino acid comprising a basic side chain. In some embodiments, a homolog of an amino acid comprising a side chain comprising a basic group, e.g., an amino group, a guanidine group, etc., is another amino acid comprising a side chain comprising a basic group.

In some embodiments, a homolog of an acidic amino acid is another acidic amino acid. In some embodiments, a homolog of an amino acid comprising an acidic side chain is another acidic amino acid comprising an acidic side chain. In some embodiments, a homolog of an amino acid comprising a side chain comprising an acidic group, e.g., —COOH, is another amino acid comprising a side chain comprising an acidic group.

In some embodiments, a homolog of an aliphatic amino acid is another aliphatic amino acid. In some embodiments, a homolog of an amino acid comprising an aliphatic side chain is another aliphatic amino acid comprising an aliphatic side chain.

In some embodiments, a homolog of an aromatic amino acid is another aromatic amino acid. In some embodiments, a homolog of an amino acid comprising an aromatic side chain is another aromatic amino acid comprising an aromatic side chain. In some embodiments, a homolog of an amino acid comprising a side chain comprising an aromatic group, e.g., phenyl, heteroaryl, etc., is another amino acid comprising a side chain comprising an aromatic group.

In some embodiments, a homolog of an amino acid is sterically similar to the amino acid. In some embodiments, a homolog of an amino acid comprises a side chain that has a similar size to the side chain of the amino acid.

Provided amino acid sequences and stapled peptides can be various lengths, e.g. 2-100, 5-50, 5-40, 5-35, a range from and including 2, 3, 4, 5, 6, or 7 to and including 8, 9, 10, 11, 12, 13, 14, 15, 16, 17, 18, 19, 20, 21, 22, 23, 24, 25, 26, 27, 28, 29, 30, 31, 32, 33, 34, or 35 amino acid residues.

In some embodiments, a length is at least 5 amino acid residues. In some embodiments, a length is at least 6 amino acid residues. In some embodiments, a length is at least 7 amino acid residues. In some embodiments, a length is at least 8 amino acid residues. In some embodiments, a length is at least 9 amino acid residues. In some embodiments, a length is at least 10 amino acid residues. In some embodiments, a length is at least 11 amino acid residues. In some embodiments, a length is at least 12 amino acid residues. In some embodiments, a length is at least 13 amino acid residues. In some embodiments, a length is at least 14 amino acid residues. In some embodiments, a length is at least 15 amino acid residues. In some embodiments, a length is at least 16 amino acid residues. In some embodiments, a length is at least 17 amino acid residues. In some embodiments, a length is at least 18 amino acid residues. In some embodiments, a length is at least 19 amino acid residues. In some embodiments, a length is at least 20 amino acid residues. In some embodiments, a length is at least 21 amino acid residues. In some embodiments, a length is at least 22 amino acid residues. In some embodiments, a length is at least 23 amino acid residues. In some embodiments, a length is at least 24 amino acid residues. In some embodiments, a length is at least 25 amino acid residues. In some embodiments, a length is at least 26 amino acid residues. In some embodiments, a length is at least 27 amino acid residues. In some embodiments, a length is at least 28 amino acid residues. In some embodiments, a length is at least 29 amino acid residues. In some embodiments, a length is at least 30 amino acid residues. In some embodiments, a length is at least 31 amino acid residues. In some embodiments, a length is at least 32 amino acid residues. In some embodiments, a length is at least 33 amino acid residues. In some embodiments, a length is at least 34 amino acid residues. In some embodiments, a length is at least 35 amino acid residues.

In some embodiments, a length is 5 amino acid residues. In some embodiments, a length is 6 amino acid residues. In some embodiments, a length is 7 amino acid residues. In some embodiments, a length is 8 amino acid residues. In some embodiments, a length is 9 amino acid residues. In some embodiments, a length is 10 amino acid residues. In some embodiments, a length is 11 amino acid residues. In some embodiments, a length is 12 amino acid residues. In some embodiments, a length is 13 amino acid residues. In some embodiments, a length is 14 amino acid residues. In some embodiments, a length is 15 amino acid residues. In some embodiments, a length is 16 amino acid residues. In some embodiments, a length is 17 amino acid residues. In some embodiments, a length is 18 amino acid residues. In some embodiments, a length is 19 amino acid residues. In some embodiments, a length is 20 amino acid residues. In some embodiments, a length is 21 amino acid residues. In some embodiments, a length is 22 amino acid residues. In some embodiments, a length is 23 amino acid residues. In some embodiments, a length is 24 amino acid residues. In some embodiments, a length is 25 amino acid residues. In some embodiments, a length is 26 amino acid residues. In some embodiments, a length is 27 amino acid residues. In some embodiments, a length is 28 amino acid residues. In some embodiments, a length is 29 amino acid residues. In some embodiments, a length is 30 amino acid residues. In some embodiments, a length is 31 amino acid residues. In some embodiments, a length is 32 amino acid residues. In some embodiments, a length is 33 amino acid residues. In some embodiments, a length is 34 amino acid residues. In some embodiments, a length is 35 amino acid residues.

In some embodiments, a length is no more than 17 amino acid residues. In some embodiments, a length is no more than 18 amino acid residues. In some embodiments, a length is no more than 19 amino acid residues. In some embodiments, a length is no more than 20 amino acid residues. In some embodiments, a length is no more than 21 amino acid residues. In some embodiments, a length is no more than 22 amino acid residues. In some embodiments, a length is no more than 23 amino acid residues. In some embodiments, a length is no more than 24 amino acid residues. In some embodiments, a length is no more than 25 amino acid residues. In some embodiments, a length is no more than 26 amino acid residues. In some embodiments, a length is no more than 27 amino acid residues. In some embodiments, a length is no more than 28 amino acid residues. In some embodiments, a length is no more than 29 amino acid residues. In some embodiments, a length is no more than 30 amino acid residues. In some embodiments, a length is no more than 35 amino acid residues. In some embodiments, a length is no more than 40 amino acid residues. In some embodiments, a length is no more than 50 amino acid residues.

Both naturally occurring and non-naturally occurring amino acids can be utilized in accordance with the present disclosure. In some embodiments, an amino acid is a compound comprising an amino group that can form an amide group with a carboxyl group and a carboxyl group.

In some embodiments, an amino acid is a compound having the structure of formula A-I:

NH(R^{a1})—L^{a1}-C(R^{a2})(R^{a3})-L^{a2}-COOH, A-I

or a salt thereof, wherein:
each of R^{a1}, R^{a2}, R^{a3} is independently -L^{a}-R′;
each of L^{a}, L^{a1} and L^{a2} is independently L;
each L is independently a covalent bond, or an optionally substituted, bivalent C₁-C₂₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L^{a1} is a covalent bond. In some embodiments, a compound of formula A-1 is of the structure NH(R^{a1})—C(R^{a2})(R^{a3})-L^{a2}-COOH.

In some embodiments, L^{a2} is a covalent bond. In some embodiments, a compound of formula A-1 is of the structure NH(R^{a1})—C(R^{a2})(R^{a3})-L^{a2}-COOH.

In some embodiments, L^{a1} is a covalent bond and L^{a2} is a covalent bond. In some embodiments, a compound of formula A-1 is of the structure NH(R^{a1})—C(R^{a2})(R^{a3})—COOH.

In some embodiments, L^{a} is a covalent bond. In some embodiments, R′ is R. In some embodiments, R^{a1} is R, wherein R is as described in the present disclosure. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a}3 is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

An amino acid sequence can have various degrees of homology to naturally occurring amino acid sequences. As appreciated by those skilled in the art, the present disclosure can be utilized to design and identify sequences with either high or low homology, or no homology (e.g., sequences completely unrelated to any sequences encoded in a genome). In some embodiments, the present disclosure provides technologies for identifying useful amino acid sequences that share low degree of homology to naturally occurring amino acid sequences, e.g., from libraries designed with many random positions compared to natural amino acid sequences, or from totally random libraries. In some embodiments, an amino acid sequence is at least 5%, 10%, 20%, 30%, 40%, 50%, 60%, 70%, 80%, 90%, or 95% homologous to naturally occurring amino acid sequence. In some embodiments, an amino acid sequence is no more than 5%, 10%, 20%, 30%, 40%, 50%, 60%, 70%, 80%, 90%, or 95% homologous to naturally occurring amino acid sequence. In some embodiments, the homology is at least 5%. In some embodiments, the homology is at least 10%. In some embodiments, the homology is at least 20%. In some embodiments, the homology is at least 30%. In some embodiments, the homology is at least 40%. In some embodiments, the homology is at least 50%. In some embodiments, the homology is at least 60%. In some embodiments, the homology is at least 70%. In some embodiments, the homology is at least 80%. In some embodiments, the homology is at least 90%. In some embodiments, the homology is at least 95%. In some embodiments, the homology is no more than 5%. In some embodiments, the homology is no more than 10%. In some embodiments, the homology is no more than 20%. In some embodiments, the homology is no more than 30%. In some embodiments, the homology is no more than 40%. In some embodiments, the homology is no more than 50%. In some embodiments, the homology is no more than 60%. In some embodiments, the homology is no more than 70%. In some embodiments, the homology is no more than 80%. In some embodiments, the homology is no more than 90%. In some embodiments, the homology is no more than 95%.

An amino acid sequence can be various lengths. In some embodiments, a length is or comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 amino acid residues. In some embodiments, a length is or comprises at least 5 amino acid residues. In some embodiments, a length is or comprises at least 6 amino acid residues. In some embodiments, a length is or comprises at least 7 amino acid residues. In some embodiments, a length is or comprises at least 8 amino acid residues. In some embodiments, a length is or comprises at least 9 amino acid residues. In some embodiments, a length is or comprises at least 10 amino acid residues. In some embodiments, a length is or comprises at least 11 amino acid residues. In some embodiments, a length is or comprises at least 12 amino acid residues. In some embodiments, a length is or comprises at least 13 amino acid residues. In some embodiments, a length is or comprises at least 14 amino acid residues. In some embodiments, a length is or comprises at least 15 amino acid residues. In some embodiments, a length is or comprises at least 16 amino acid residues. In some embodiments, a length is or comprises at least 17 amino acid residues. In some embodiments, a length is or comprises at least 18 amino acid residues. In some embodiments, a length is or comprises at least 18 amino acid residues. In some embodiments, a length is or comprises at least 20 amino acid residues. In some embodiments, a length is or comprises at least 25 amino acid residues. In some embodiments, a length is or comprises at least 30 amino acid residues.

As demonstrated herein, provided technologies can be utilized with or without a starting amino acid sequence (e.g., either totally random libraries or libraries comprising certain pre-determined amino acid residues at certain positions) to identify useful amino acid sequences with desired properties and/or activities. In some embodiments, the present disclosure provide useful amino acid sequences for modulating targets, e.g., proteins associated with various conditions, disorders or diseases. In some embodiments, a provided amino acid sequence comprises Xₛ₁X₁X₂X₃X₄X₅X₆Xₛ₂, wherein Xₛ₁ and Xₛ₂ are amino acid residues connected via a staple (stapled) or suitable for stapling, and each of X₁, X₂, X₃, X₄, X₅, and X₆ is independently an amino acid residue. In some embodiments, X₃ and X₄ are A, and X₅ is H. In some embodiments, X₂ is L, X₃ and X₄ are A, and X₅ is H. In some embodiments, X₂ is L. In some embodiments, X₁ is I. In some embodiments, a provided amino acid sequence comprises Xₛ₁X₁X₂X₃X₄X₅X₆Xₛ₂X₇X₈, wherein Xₛ₁ and Xₛ₂ are amino acid residues connected via a staple (stapled) or suitable for stapling, and each of X₁, X₂, X₃, X₄, X₅, X₆, X₇, and X₈ is independently an amino acid residue. In some embodiments, X₃ and X₄ are A, and X₆ is H. In some embodiments, X₂ is an aromatic amino acid residue, X₃ and X₄ are A, and X₆ is H. In some embodiments, X₂ is W. In some embodiments, X₁ is E. In some embodiments, X₇ is an acidic amino acid residue. In some embodiments, X₇ is E. In some embodiments, X₈ is L. In some embodiments, a provided amino acid sequence comprises X₋₂X₋₁Xₛ₁X₁X₂X₃X₄X₅X₆Xₛ₂X₇, wherein Xₛ₁ and Xₛ₂ are amino acid residues connected via a staple, and each of X₋₂, X₋₁, X₁, X₂, X₃, X₄, X₅, X₆, and X₇ is independently an amino acid residue. In some embodiments, X₂ is H, X₃ is A, and X₄ is A. In some embodiments, X₋₂ is W. In some embodiments, X₋₁ is an acidic amino acid residue. In some embodiments, X₋₁ is E. In some embodiments, X₋₁ is D. In some embodiments, X₁ is an acidic amino acid residue. In some embodiments, X₁ is E. In some embodiments, X₁ is D. In some embodiments, X₅ is L or I. In some embodiments, X₅ is L. In some embodiments, X₅ is I. In some embodiments, X₆ is L or I. In some embodiments, X₆ is L. In some embodiments, X₆ is I. In some embodiments, X₇ is an acidic amino acid residue. In some embodiments, X₇ is E. In some embodiments, X₇ is D.

Certain useful amino acids, including those useful for stapling or are stapled, are described in U.S. Ser. No. 11/198,713, US 20210179665, WO 2021119537, WO 2021188659, WO 2022020651, or WO 2022020652, the entirety of each of which is incorporated herein by reference.

### Peptide Libraries/Collections

In some embodiments, the present disclosure provides collections of peptides. In some embodiments, a collection of peptides is a collection of stapled peptides. In some embodiments, peptides within a collection as described herein may share one or more structural features (e.g. length within a particular range; particular lengths, presence of particular sequence elements such as, for example, a sequence element found in a known interaction partner for a target of interest, a set of amino acids interacting with a target of interest, or a set of amino acids that together support formation of a staple [e.g., two or more cysteine residues, positioned relative to one another so that a cysteine staple as described herein is or may be produced between a pair of them], presence of one or more staples which may, in some embodiments, be of the same type, etc., or any combination thereof, and in some embodiments, a particular collection may be characterized and/or defined by such shared structural feature(s)). In some embodiments, a common structural feature of peptides in a collection of peptides as described herein is at least two stapled residues, or at least two residues suitable for stapling, positioned relative to one another so that a staple as described herein is or may be formed between a pair of them. In some embodiments, two residues are cysteine residues. In some embodiments, such peptides can be reacted with a compound of formula R-I, to produce a collection of stapled peptides.

In some embodiments, a collection of peptides is a collection of stapled peptides, each of which independently has an amino acid sequence that:
has a length within a range of a* and b*, where a* and b* are each integers independently selected from 2 through 100 and b* is greater than a*; and
includes at least one pair of residues covalently linked with one another via a linker.

In some embodiments, a* and b* are each integers independently selected from 2 through 50 and b* is greater than a*. In some embodiments, a* and b* are each integers independently selected from 6 through 36 and b* is greater than a*.

In some embodiments, peptides in a collection are of the same or about the same length. In some embodiments, about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% peptides of a collection are of the same length. In some embodiments, a length is about a* as described herein. In some embodiments, a length is about b* as described herein.

In some embodiments, a* is 1. In some embodiments, a* is 2. In some embodiments, a* is 3. In some embodiments, a* is 4. In some embodiments, a* is 5. In some embodiments, a* is 6. In some embodiments, a* is 7. In some embodiments, a* is 8. In some embodiments, a* is 9. In some embodiments, a* is 10. In some embodiments, a* is 11. In some embodiments, a* is 12. In some embodiments, a* is 13. In some embodiments, a* is 14. In some embodiments, a* is 15. In some embodiments, a* is 16. In some embodiments, a* is 17. In some embodiments, a* is 18. In some embodiments, a* is 19. In some embodiments, a* is 20. In some embodiments, a* is 21. In some embodiments, a* is 22. In some embodiments, a* is 23. In some embodiments, a* is 24. In some embodiments, a* is 25. In some embodiments, a* is 26. In some embodiments, a* is 27. In some embodiments, a* is 28. In some embodiments, a* is 29. In some embodiments, a* is 30. In some embodiments, a* is 31. In some embodiments, a* is 32. In some embodiments, a* is 33. In some embodiments, a* is 34. In some embodiments, a* is 35. In some embodiments, a* is 36. In some embodiments, a* is 37. In some embodiments, a* is 38. In some embodiments, a* is 39. In some embodiments, a* is 40. In some embodiments, a* is 41. In some embodiments, a* is 42. In some embodiments, a* is 43. In some embodiments, a* is 44. In some embodiments, a* is 45. In some embodiments, a* is 46. In some embodiments, a* is 47. In some embodiments, a* is 48. In some embodiments, a* is 49.

In some embodiments, b* is 2. In some embodiments, b* is 3. In some embodiments, b* is 4. In some embodiments, b* is 5. In some embodiments, b* is 6. In some embodiments, b* is 7. In some embodiments, b* is 8. In some embodiments, b* is 9. In some embodiments, b* is 10. In some embodiments, b* is 11. In some embodiments, b* is 12. In some embodiments, b* is 13. In some embodiments, b* is 14. In some embodiments, b* is 15. In some embodiments, b* is 16. In some embodiments, b* is 17. In some embodiments, b* is 18. In some embodiments, b* is 19. In some embodiments, b* is 20. In some embodiments, b* is 21. In some embodiments, b* is 22. In some embodiments, b* is 23. In some embodiments, b* is 24. In some embodiments, b* is 25. In some embodiments, b* is 26. In some embodiments, b* is 27. In some embodiments, b* is 28. In some embodiments, b* is 29. In some embodiments, b* is 30. In some embodiments, b* is 31. In some embodiments, b* is 32. In some embodiments, b* is 33. In some embodiments, b* is 34. In some embodiments, b* is 35. In some embodiments, b* is 36. In some embodiments, b* is 37. In some embodiments, b* is 38. In some embodiments, b* is 39. In some embodiments, b* is 40. In some embodiments, b* is 41. In some embodiments, b* is 42. In some embodiments, b* is 43. In some embodiments, b* is 44. In some embodiments, b* is 45. In some embodiments, b* is 46. In some embodiments, b* is 47. In some embodiments, b* is 48. In some embodiments, b* is 49.

In some embodiments, a pair of residues covalently linked with one another via a linker is covalently linked via a staple. In some embodiments, a pair of residues covalently linked with one another via a linker covalently linked via a non-hydrocarbon staple. In some embodiments, a pair of residues covalently linked with one another via a linker is covalently linked with via a cysteine staple. In some embodiments, a cysteine staple has the structure as described in the present disclosure. In some embodiments, a staple has the structure of -L^{s1}-S-L^{s2}-S-L^{s3}- wherein each variable is independently as described herein. In some embodiments, a staple has the structure of L as described herein.

In some embodiments, the present disclosure provides a collection of stapled peptides, each of which independently has an amino acid sequence that: has a length within a range of a* and b*, where a* and b* are integers selected from 2 through 100 inclusive and b* is greater than a*; comprises a pair of residues covalently linked with one another via a linker; and residues of the pair are separated by c* residues, where c* is an integer selected from 0 through 12.

In some embodiments, a linker is a staple as described herein. In some embodiments, a linker is -L^{s1}-S-L^{s2}-S-L^{s3}- wherein each variable is independently as described herein. In some embodiments, a linker is L as described herein.

In some embodiments, the present disclosure provides a collection of stapled peptides, each of which independently has an amino acid sequence that:
has a length within a range of a* and b*, where a* and b* are integers selected from 2 through 100 inclusive and b* is greater than a*;
includes at least one pair of cysteine residues covalently linked with one another via a linker comprising a moiety —S-L^{s2}-S—, where each S is independently a sulfur atom of a cysteine residue;
L^{s2} is described in the present disclosure; and
cysteine residues of a pair are independently separated by c* residues, where c* is an integer selected from 0 through 12.

In some embodiments, a pair of cysteine residues covalently linked with one another via linker are separated by c* residues, wherein c* is an integer 1 to 12 inclusive. In some embodiments, c* is 1. In some embodiments, c* is 2. In some embodiments, c* is 3. In some embodiments, c* is 4. In some embodiments, c* is 5. In some embodiments, c* is 6. In some embodiments, c* is 7. In some embodiments, c* is 8. In some embodiments, c* is 9. In some embodiments, c* is 10. In some embodiments, c* is 11. In some embodiments, c* is 12.

In some embodiments, the present disclosure provides a collection of stapled peptides of the structure:
[X¹]ₚ₁[X²]ₚ₂[X³]ₚ₃[X⁴]ₚ₄[X⁵]ₚ₅[X⁶]ₚ₆[X⁷]ₚ₇[X⁸]ₚ₈[X⁹]ₚ₉[X¹⁰]ₚ₁₀[X¹¹]ₚ₁₁[X¹²]ₚ₁₂[X¹³]ₚ₁₃-X¹⁴X¹⁵X¹⁶X¹⁷X¹⁸X¹⁹-[X²⁰]ₚ₂₀[X²¹]ₚ₂₁[X²²]ₚ₂₂[X²³]ₚ₂₃[X²⁴]ₚ₂₄[X²⁵]ₚ₂₅[X²⁶]ₚ₂₆[X²⁷]ₚ₂₇[X²⁸]ₚ₂₈[X²⁹]ₚ₂₉[X³⁰]ₚ₃₀[X³¹]ₚ₃₁[X³²]ₚ₃₂,
wherein:
   each of p1, p2, p3, p4, p5, p6, p7, p8, p9, p10, p11, p12, p13, p20, p21, p22, p23, p24, p25, p26, p27, p28, p29, p30, p31, p32 is independently 0 or 1;
   each of X¹ to X³² is independently an amino acid residue;
   at least two of X¹ to X³² are covalently linked with one another via a linker.

In some embodiments, the present disclosure provides a collection of stapled peptides of the structure:
[X¹]ₚ₁[X²]ₚ₂[X³]ₚ₃[X⁴]ₚ₄[X⁵]ₚ₅[X⁶]ₚ₆[X⁷]ₚ₇[X⁸]ₚ₈[X⁹]ₚ₉[X¹⁰]ₚ₁₀[X¹¹]ₚ₁₁[X¹²]ₚ₁₂[X¹³]ₚ₁₃-X¹⁴X¹⁵X¹⁶X¹⁷X¹⁸X¹⁹-[X²⁰]ₚ₂₀[X²¹]ₚ₂₁[X²²]ₚ₂₂[X²³]ₚ₂₃[X²⁴]ₚ₂₄[X²⁵]ₚ₂₅[X²⁶]ₚ₂₆[X²⁷]ₚ₂₇[X²⁸]ₚ₂₈[X²⁹]ₚ₂₉[X³⁰]ₚ₃₀[X³¹]ₚ₃₁[X³²]ₚ₃₂,
wherein:
   each of p1, p2, p3, p4, p5, p6, p7, p8, p9, p10, p11, p12, p13, p20, p21, p22, p23, p24, p25, p26, p27, p28, p29, p30, p31, p32 is independently 0 or 1;
   each of X¹ to X³² is independently an amino acid residue;
   at least two of X¹ to X³² comprise cysteine chains that are optionally linked together to form a cysteine staple.

In some embodiments, each X^{j} is independently selected from the group of standard amino acids, wherein j is an integer from 1 to 32 inclusive. In some embodiments, X^{j} is selected from a subset of standard amino acids. In some embodiments, X^{j} is selected from a group of all natural amino acids except cysteine. In some embodiments, X^{j} is selected from fewer than 20 of the standard amino acids. In some embodiments, X^{j} is selected from fewer than 19 of the standard amino acids. In some embodiments, X^{j} is selected from fewer than 18 of the standard amino acids. In some embodiments, X^{j} is selected from fewer than 17 of the standard amino acids. In some embodiments, X^{j} is selected from fewer than 16 of the standard amino acids. In some embodiments, X^{j} is selected from fewer than 15 of the standard amino acids. In some embodiments, X^{j} is selected from fewer than 14 of the standard amino acids. In some embodiments, X^{j} is selected from fewer than 13 of the standard amino acids. In some embodiments, X^{j} is selected from fewer than 12 of the standard amino acids. In some embodiments, X^{j} is selected from fewer than 11 of the standard amino acids. In some embodiments, X^{j} is selected from fewer than 10 of the standard amino acids. In some embodiments, X^{j} is selected from fewer than 9 of the standard amino acids. In some embodiments, X^{j} is selected from fewer than 8 of the standard amino acids. In some embodiments, X^{j} is selected from fewer than 7 of the standard amino acids. In some embodiments, X^{j} is selected from fewer than 6 of the standard amino acids. In some embodiments, X^{j} is selected from fewer than 5 of the standard amino acids. In some embodiments, X^{j} is selected from fewer than 4 of the standard amino acids. In some embodiments, X^{j} is selected from fewer than 3 of the standard amino acids. In some embodiments.

In some embodiments, each X^{j} is independently selected from 20 of the standard amino acids. In some embodiments, X^{j} is selected from 19 of the standard amino acids. In some embodiments, X^{j} is selected from 18 of the standard amino acids. In some embodiments, X^{j} is selected from 17 of the standard amino acids. In some embodiments, X^{j} is selected from 16 of the standard amino acids. In some embodiments, X^{j} is selected from 15 of the standard amino acids. In some embodiments, X^{j} is selected from 14 of the standard amino acids. In some embodiments, X^{j} is selected from 13 of the standard amino acids. In some embodiments, X^{j} is selected from 12 of the standard amino acids. In some embodiments, X^{j} is selected from 11 of the standard amino acids. In some embodiments, X^{j} is selected from 10 of the standard amino acids. In some embodiments, X^{j} is selected from 9 of the standard amino acids. In some embodiments, X^{j} is selected from 8 of the standard amino acids. In some embodiments, X^{j} is selected from 7 of the standard amino acids. In some embodiments, X^{j} is selected from 6 of the standard amino acids. In some embodiments, X^{j} is selected from 5 of the standard amino acids. In some embodiments, X^{j} is selected from 4 of the standard amino acids. In some embodiments, X^{j} is selected from 3 of the standard amino acids. In some embodiments, X^{j} is selected from 2 of the standard amino acids. In some embodiments, X^{j} is selected from 1 of the standard amino acids.

In some embodiments, j is 1. In some embodiments, j is 2. In some embodiments, j is 3. In some embodiments, j is 4. In some embodiments, j is 5. In some embodiments, j is 6. In some embodiments, j is 7. In some embodiments, j is 8. In some embodiments, j is 9. In some embodiments, j is 10. In some embodiments, j is 11. In some embodiments, j is 12. In some embodiments, j is 13. In some embodiments, j is 14. In some embodiments, j is 15. In some embodiments, j is 16. In some embodiments, j is 17. In some embodiments, j is 18. In some embodiments, j is 19. In some embodiments, j is 20. In some embodiments, j is 21. In some embodiments, j is 22. In some embodiments, j is 23. In some embodiments, j is 24. In some embodiments, j is 25. In some embodiments, j is 26. In some embodiments, j is 27. In some embodiments, j is 28. In some embodiments, j is 29. In some embodiments, j is 30. In some embodiments, j is 31. In some embodiments, j is 32.

Those skilled in the art, reading the present disclosure, will appreciate that peptide collections as described herein can be prepared, provided and/or utilized in a variety of formats. In some embodiments, a peptide collection is prepared, provided, and or utilized in a format such as, for example, phage display, yeast display, bacteria display, ribosome display, mRNA display, on a solid support, on a solid phase, on a resin, in liquid solution, as a dried sample or set thereof, etc.

In some embodiments, a collection of peptides can be provided and or utilized in phage display. In some embodiments, a collection of peptides are fused to a phage protein. In some embodiments, a collection of peptides are fused to a phage coat protein. In some embodiments, a collection of peptides are fused to a phage coat protein pIII.

Peptide collections may be displayed using a number of technologies. In some embodiments, peptides of libraries are displayed on p4 of M13 phage. In some embodiments, peptides of libraries are displayed on p7 of M13 phage. In some embodiments, peptides of libraries are displayed on p8 of M13 phage. In some embodiments, peptides of libraries are displayed on p9 of M13 phage.

In some embodiments, libraries, e.g., phage libraries, may be prepared with one or more barcodes within the DNA sequence of the library members, for example, comprising “silent” mutations (e.g., distinct codons that all encode the same amino acid), amino acid mutations, etc. In some embodiments, barcodes are used to identify certain features of library members and/or certain features of uses, e.g., experiments they are screened in, so that those features may be associated with the library member screening results, e.g., during analyses of DNA sequencing outputs of the screens. For example, in some embodiments, barcodes are incorporated into multiple libraries so that those libraries can be screened together, and their results are identified (and separated) by use of the barcodes. In some embodiments, barcodes are used to identify features of processes, e.g., experiments that a library member were screened in. Exemplary features include targets (e.g., proteins, nucleic acids, cells, etc.), buffer conditions, binding partners, competitors included in screens, temperatures of experiments, duration of experiments, washing procedures, and/or other features of experimental procedures. In some embodiments, barcodes are utilized to identify crosslinkers, e.g., those used to prepare chemically modified phage libraries comprising stapled peptides.

Various technologies can be utilized to incorporate unnatural amino acids into peptides, collections (e.g., various libraries described herein), etc., in accordance with the present disclosure. In some embodiments, incorporation comprises the use of codon suppression and/or aminoacyl-tRNA synthetase /tRNA pairs that result in the incorporation of unnatural amino acids. In some embodiments, useful methods comprise the addition of glyphosate (or other agents that selectively suppress the biosynthesis of one or more amino acids) and unnatural amino acid(s) in growth media. In some embodiments, useful methods comprise the use of cell lines lacking the ability to synthesize certain amino acids and the addition of unnatural amino acid(s) in growth media. In some embodiments, useful methods comprise the addition of unnatural amino acid(s) in growth media. In some embodiments, barcodes are used to identify the incorporation of unnatural amino acids into library designs.

In some embodiments, known phage or DNA sequence(s) are added during a step of a phage screen, for the purpose of scaling or normalizing DNA sequencing data using the known sequence(s) (“spike-in” samples).

In some embodiments, in the context of a collection of peptides (or proteins, etc.; e.g., of a provided library), diversity may refer to either a) the actual number of unique amino acid sequences present in said collection of peptides or b) the theoretical number of unique amino acid sequences that could exist, e.g., based on design and/or preparation of the collection. In some embodiments, diversity is actual diversity, e.g., as measured and described in the Examples.

In some embodiments, a peptide collection as described herein is characterized by an actual diversity of at least 1×10⁴ unique peptide sequences. In some embodiments, a peptide collection is characterized by an actual diversity of at least 1×10⁵ unique peptide sequences. In some embodiments, a peptide collection is characterized by an actual diversity of at least 1×10⁶ unique peptide sequences. In some embodiments, a peptide collection is characterized by an actual diversity of at least 1×10⁷ unique peptide sequences. In some embodiments, a peptide collection is characterized by an actual diversity of at least 1×10⁸ unique peptide sequences. In some embodiments, a peptide collection is characterized by an actual diversity of at least 1×10⁹ unique peptide sequences. In some embodiments, a peptide collection is characterized by an actual diversity of at least 1×10¹⁰ unique peptide sequences. In some embodiments, a peptide collection is characterized by an actual diversity of at least 1×10¹¹ unique peptide sequences. In some embodiments, a peptide collection is characterized by an actual diversity of at least 1×10¹² unique peptide sequences. In some embodiments, a peptide collection is characterized by an actual diversity of at least 1×10¹³ unique peptide sequences. In some embodiments, a peptide collection is characterized by an actual diversity of at least 1×10¹⁴ unique peptide sequences. In some embodiments, a peptide collection is characterized by an actual diversity of at least 1×10¹⁵ unique peptide sequences. In some embodiments, a preferred actual diversity for a peptide collection e.g., of a library, is 10⁸-10⁹ unique peptide sequences. In some embodiments, such a collection may have a theoretical diversity that is often 1,000-10,000-fold greater than the actual diversity, for example, 10 positions with 16 amino acid possibilities at each position can have a theoretical diversity of 10¹²; depending on procedures, purposes, etc., a collection of peptides, e.g., of a library may be prepared with a subset of the theoretical diversity, e.g., of 10⁸ unique sequences.

In some embodiments, one or more (e.g., about or at least about 1, 2, 3, 4, 5, 6, 7, 8, 9, or 10, or about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of all amino acid residues), or each of the amino acid residue positions are randomized (e.g., at degenerate positions). In some embodiments, at a randomized position a variety of amino acid residues are independently presented in a collection of peptides (e.g., a library described in an example). In some embodiments, each position is randomized. In some embodiments, a collection is provided without amino acid residue preferences at any positions (e.g., in some embodiments, a “naive” or “unbiased” collection/library). As appreciated by those skilled in the art, incorporation of randomized positions in some embodiments can be done by encoding a degenerate codon in an oligonucleotide primer used to generate a library, for example, NNN or NNK where N=A, T, C, G and K=T, G. Alternatively, randomized positions can be incorporated by the use of trimer phosphoroamidite mixtures, e.g., those available from Glen research, wherein a defined mixture of trimer codons that encode a corresponding defined mixture of amino acids are incorporated in the oligonucleotide primer used to generate the library. For example, a library could be constructed using a mixture of all 20 naturally occurring amino acids, or alternatively by using a subset of all 20 naturally occurring amino acids (e.g., A, D, E, F, H, I, L, M, N, Q, R, S, T, V, W, Y, etc.). In some embodiments, non-natural amino acids may also be incorporated using various technologies.

In some embodiments, randomized positions can be incorporated through chemical synthesis, e.g., by utilizing mixtures of amino acids at such positions. In some embodiments, amino acids are present at the same levels in mixtures. In some embodiments, certain amino acids may have higher levels than others, e.g., in view of synthesis efficiency.

In some embodiments, an amino acid residue at a randomized, degenerate, or not biased or enriched position (e.g., in a cluster as described herein) is selected from alanine, arginine, asparagine, aspartic acid, glutamine, glutamic acid, histidine, isoleucine, leucine, methionine, phenylalanine, serine, threonine, tryptophan, tyrosine, and valine.

In some embodiments, peptides of a collection, e.g., a library, comprise fixed or a subset of preferred amino acids at one or more positions for one or more desired property and/or activity, e.g., in some embodiments, to favor an alpha-helical conformation. For example, incorporation of alanine residues at defined positions in the middle of the peptide (e.g. library designs b), e), and h) above), a proline at the N-terminus (e.g. library designs g), h), i) above), or an aspartic acid followed by a proline at the N-terminus (e.g. library designs d), e), f) in the list above). In some embodiments, a desired property or activity is binding to a particular target (e.g., protein, nucleic acid, etc.). In some embodiments, a desired property or activity is cell penetration. In some embodiments, a desired property or activity is stability (e.g., to proteases or other types of degradation). In some embodiments, a desired property or activity is low immunogenicity. In some embodiments, a desired property or activity is improved physicochemical property. In some embodiments, a desired property or activity is improved pharmacokinetic properties. In some embodiments, a desired property or activity is selectivity for a target or tissue. As those skilled in the art will appreciate, fixed and/or a subset of preferred amino acids may be independently presented at each of one or more positions to provide one or more desired properties and/or activities.

In some embodiments, peptides of a collection comprise stapled residues or residues suitable for stapling at certain positions.

In some embodiments, peptides of a collection comprise enriched amino acid residues useful for binding to a target of interest. In some embodiments, such enriched amino acid residues interact with a target of interest. In some embodiments, peptides of such a collection comprise one or more, e.g., about or at least about 1, 2, 3, 4, 5, 6, 7, 8, 9, or 10, randomized positions. In some embodiments, such a collection are useful for identifying agents that can bridge two targets of interest, wherein the enriched amino acid residues facility interactions with a first target of interest (e.g., a presenter protein), wherein the randomized positions can provide diversity for binding to a second target of interest.

In some embodiments, collections, e.g., peptide libraries, can be designed on the basis of one or more preferred or parent sequences. In some embodiments, a parent sequence is systematically diversified to give rise to many collection members. In some embodiments, a parent sequence is subjected to an “alanine scan” where each residue within the sequence is changed to alanine, either individually or in combination with other changes, mutations and/or modifications, to provide a number of peptides in a collection. In some embodiments, alanine scan is useful for assessing importance of one or more residues for certain properties and/or activities, e.g., binding, interactions, stability, physicochemical properties, cell penetration, immunogenicity, selectivity (e.g., for a target (e.g., protein, nucleic acid, etc.), tissue, etc.), pharmacokinetic properties, etc. A parent sequence could similarly be systematically changed/mutated to proline or glycine e.g., for assessing the effect of structural and/or conformational changes. A parent sequence could also be systematically changed/mutated to charged residues, e.g., for interrogating roles of charge in binding, stability, physicochemical properties, cell penetration, immunogenicity, selectivity, pharmacokinetic properties, etc. A parent sequence could also be systematically changed/mutated at each position to a variety of amino acids, e.g., for identifying point-mutations with improved properties and/or activities, e.g., binding, stability, physicochemical properties, cell penetration, immunogenicity, selectivity, pharmacokinetic properties, etc. As those skilled in the art will appreciate, collections can be designed using various established methods of library mutagenesis and directed evolution in accordance with the present disclosure.

In some embodiments, a parent sequence is pancreatic polypeptide, neuropeptide Y, or peptide YY, or a fragment thereof. In some embodiments, a fragment has a length of 5, 6, 7, 8, 9, 10, 11, 12, 13, 14, 15, 16, 17, 18, 19, 20, or more, amino acid residues. For example, in some embodiments, a sequence is selected from:

TABLE Q-1

| Species | Sequence |
|---|---|
| Homo sapiens | PSKPDNPGEDAPAEDMARYYSALGHYINLIT (SEQ ID NO: 6) |
| Ictalurus punctatus | PSKPENPGEDAAPEELAKYYTALRHYINLIT (SEQ ID NO: 7) |
| Marmota marmota | PLEPVYPGDNATPEQMAQYAAELRRYINMLT (SEQ ID NO: 8) |
| Myotis lucifugus | PSKPEKPGENASAEELARYYSALRHYINLIT (SEQ ID NO: 9) |
| Betta splendens | PPKPENPGEDAPPEELAKYYTALRHYINLIT (SEQ ID NO: 10) |
| Notechis scutatus | PPKPESPGENASPEEMAKYLADLRHYINLVT (SEQ ID NO: 11) |
| Orcinus orca | PAKPEAPGSHASPEELKRYYLSLRHFLNLVT (SEQ ID NO: 12) |
| Calypte anna | PPKPETPGDEASPEEVAKYFSALRHYINLVT (SEQ ID NO: 13) |
| Podarcis muralis | PQQPEHPGEDASAEEMARYLSALRHYLNLVT (SEQ ID NO: 14) |
| Vombatus ursinus | PSKPKPPSENASREELSRYYAALRQYLNLVT (SEQ ID NO: 15) |
| Ornithorhynchus anatinus | PVKPQPPPDNATPEELAQYFASLRHYLNLVT (SEQ ID NO: 16) |
| Otolemur garnettii | PLEPVYPGENATPEQMAQYAAELRRYINMLT (SEQ ID NO: 17) |
| Gulo gulo | PSKPDNPGEDAPAEDMARYYSALRHYINLIT (SEQ ID NO: 18) |
| Crocodylus porosus | PSKPDNPGEDAPAEDMARYYSALRHYINLIT (SEQ ID NO: 915) |

In some embodiments, a parent sequence is a sequence that shares certain level of homology with another sequence, e.g., pancreatic polypeptide sequences above. In some embodiments, a level is 85%, 90%, 95% or more. In some embodiments, a level is 90%. In some embodiments, a level is 95%. In some embodiments, amino acid sequences of stapled peptides are the same as parent sequences except one or more residues of the parent sequences are replaced with residues for stapling (e.g., cysteine residues for cysteine stapling as described herein). In some embodiments, amino acid sequences of stapled peptides are the same as parent sequences except two or more residues of the parent sequences are replaced with residues for stapling. In some embodiments, amino acid sequences of stapled peptides are the same as parent sequences except two residues of the parent sequences are replaced with residues for stapling.

In some embodiments, collections of peptides, e.g., various libraries, are based on pancreatic polypeptides. In some embodiments, libraries of pancreatic polypeptides comprise randomization of one or more residues in an alpha-helical region. In some embodiments, libraries of pancreatic polypeptide, neuropeptide Y, or peptide YY comprise randomization of one or more residues in the PPII region. In some embodiments, libraries of pancreatic polypeptide, neuropeptide Y, or peptide YY comprise randomization of one or more residues in a loop region. In some embodiments, libraries of pancreatic polypeptide, neuropeptide Y, or peptide YY comprise randomization of one or more residues in two or more regions of the peptide. In some embodiments, such libraries are prepared by randomizing residues one or more regions.

In some embodiments, pancreatic polypeptide, neuropeptide Y, or peptide YY is from Homo sapiens. In some embodiments, pancreatic polypeptide, neuropeptide Y, or peptide YY is from Ictalurus punctatus. In some embodiments, pancreatic polypeptide, neuropeptide Y, or peptide YY is from Marmota marmota. In some embodiments, pancreatic polypeptide, neuropeptide Y, or peptide YY is from Myotis lucifugus. In some embodiments, pancreatic polypeptide, neuropeptide Y, or peptide YY is from Betta splendens. In some embodiments, pancreatic polypeptide, neuropeptide Y, or peptide YY is from Notechis scutatus. In some embodiments, pancreatic polypeptide, neuropeptide Y, or peptide YY is from Orcinus orca. In some embodiments, pancreatic polypeptide, neuropeptide Y, or peptide YY is from Calypte anna. In some embodiments, protein folds with higher thermal stability are preferred scaffolds for randomization/generation of libraries. In some embodiments, libraries, e.g., of pancreatic polypeptides, are prepared by randomizing residues of a naturally occurring pancreatic polypeptide fold with high thermal stability. In some embodiments, libraries of pancreatic polypeptides are prepared by randomizing residues of a pancreatic polypeptide fold that has been modified to improve thermal stability.

In some embodiments, the present disclosure provides methods for optimizing a peptide of a parent sequence. In some embodiments, the present disclosure provides methods comprising:
modifying a peptide by replacing each of one or more amino acid residues independently with a different amino acid residue to provide a modified peptide, wherein the modified peptide comprises at least two residues suitable for stapling; and
stapling the two residues suitable for stapling.

In some embodiments, a peptide is of or comprises a parent sequence as described herein (e.g., a peptide YY sequence or a fragment thereof). In some embodiments, the two residues suitable for stapling are cysteine residues. In some embodiments, two cysteine residues are stapled as described herein, e.g., using reagents described herein. In some embodiments, a modified peptide provides an improved properties and/or activity, e.g., improved affinity and/or selectivity for target binding, improved stability, improved helix formation, improved cell penetration, etc. In some embodiments, a collection of modified peptides are prepared, e.g., by randomizing one or more residues as described herein. In some embodiments, collections of modified peptides are provided as phase display libraries. In some embodiments, members of such collections/libraries have staples, e.g., cysteine staples, as described herein. In some embodiments, the present disclosure provides, comprising contacting provided collections, libraries or modified peptides with a target of interest so that one or more stapled peptides of the collection binds to the target, and determining amino acid sequences of stapled peptides that bind to the target as described herein.

As will be appreciated by those skilled in the art, provided technologies are applicable to various parent sequences and/or helical scaffolds. In some embodiments, a parent sequence is, or is a fragment (comprising, e.g., 5, 6, 7, 8, 9, 10, 11, 12, 13, 14, 15, 16, 17, 18, 19, 20, or more amino acid residues), or is a helical scaffold of, exendin-4, trp-cage peptides, retro trp-cage peptides, conotoxins, scyllatoxins, scorpion toxins, charybdotoxin, villin headpiece, beta-alpha-beta motif peptides, beta-beta-alpha motif peptides, mastoparan, zinc fingers, helix-linker-helix scaffolds, leucine zipper scaffolds, pancreatic polypeptide scaffolds, neuropeptide Y scaffolds, peptide YY scaffolds, mutants of the aforementioned scaffolds that have been modified for the purpose of increased helix stabilization, and other helical display scaffolds in the art. In some embodiments, peptides of provided collections comprise helix-initiating or helix-termination sequences. In some embodiments, peptides of provided collections comprise an N-terminal leader sequence such as AAA, DPA, NPA, APA, PA, P, or AP, and/or a C-terminal leader sequence such as R, RR, RP, RG, GR, PR, G, GG, P, PP, GP, or PG.

In some embodiments, cysteines are incorporated into sequences of peptides, e.g. displayed peptides using various display technologies. In some embodiments, peptides which bind a target peptide or protein at a site within close distance of one or more cysteines on the target protein may be identified via the formation a covalent bond between the cysteine on a peptide and a cysteine of the target protein (e.g., on its surface), either directly or through a linker (e.g., through using various reagents suitable for cross-linking cysteine residues), which in some embodiments can lead to an observed high-affinity and/or slow off-rate interactions in an assay (e.g., a screen assay assessing binding). Among other things, identification of such cysteines are useful for various purposes, e.g., development of agents that can interact the targets (e.g., covalent target inhibitors), use of disulfide tethering for library synthesis, compound optimization, etc. Those skilled in the art will appreciates that various uses of target cysteines, e.g., surface cysteines on targets, are available in the art.

Peptides of the present disclosure can have various sequences. Useful sequences can have various percentage and/or numbers of one or more amino acid residues. For example, in some embodiments, a sequence comprises 2 and no more than 2 cysteine residues (either stapled or not). In some embodiments, a sequence comprises more than 2 cysteine residues (either stapled or not). In some embodiments, a sequence comprises 2 and no more than 2 stapled cysteine residues. In some embodiments, a sequence comprises more than 2 stapled cysteine residues. In some embodiments, a percentage of stapled cysteine residues in a sequence is 5%-50%. In some embodiments, a percentage is no more than 5%, 6%, 7%, 8%, 9%, 10%, 11%, 12%, 13%, 14%, 15%, 20%, 25%, 30%, 35%, 40%, or 50%. In some embodiments, a percentage is about 5%, 6%, 7%, 8%, 9%, 10%, 11%, 12%, 13%, 14%, 15%, 20%, 25%, 30%, 35%, 40%, or 50%. In some embodiments, a percentage is about or no more than about 5%. In some embodiments, a percentage is about or no more than about 6%. In some embodiments, a percentage is about or no more than about 7%. In some embodiments, a percentage is about or no more than about 8%. In some embodiments, a percentage is about or no more than about 9%. In some embodiments, a percentage is about or no more than about 10%. In some embodiments, a percentage is about or no more than about 11%. In some embodiments, a percentage is about or no more than about 12%. In some embodiments, a percentage is about or no more than about 13%. In some embodiments, a percentage is about or no more than about 14%. In some embodiments, a percentage is about or no more than about 15%. In some embodiments, a percentage is about or no more than about 20%.

In some embodiments, collections of peptides, e.g., provided peptide libraries, are utilized to screen for peptides of certain properties and/or activities. Various screening technologies are described in the art and can be utilized in accordance with the present disclosure. In some embodiments, a library is screened against a target, e.g., a protein, a nucleic acid, etc. In some embodiments, all amino acid residues in a target are D configured amino acids (e.g., a mirror-image display). In some embodiments, libraries are screened against cells, e.g., cell samples, cells grown in culture, etc., or in vivo, using various technologies described in the art. In some embodiments, libraries are screened against human cells. In some embodiments, libraries are screened in a living mammal. In some embodiments, libraries are screened in a living primate.

In some embodiments, libraries are screened for binding affinity to targets, e.g., proteins, nucleic acids, etc. In some embodiments, libraries are screened for selectivity for one target (e.g., a protein) over another target (e.g., a protein). In some embodiments, libraries are screened for one or more desired characteristics such as stability, physicochemical properties, cell penetration, immunogenicity (e.g., low immunogenicity), pharmacokinetic properties, etc. In some embodiments, libraries are screened for affinity to lipids, for optimizing physicochemical properties, and/or cell penetration. In some embodiments, libraries are screened against immobilized lipids, for optimizing physicochemical properties and/or cell penetration. In some embodiments, libraries are screened for crossing blood-brain-barrier or neural barriers, either in vivo or with in vitro models of a blood-brain-barrier or neural barriers. In some embodiments, libraries are screened for crossing intestine or other relevant epithelial cells, either in vivo or with in vitro models of intestine or other relevant epithelial cells. In some embodiments, libraries are screened using organoid models.

In some embodiments, screens are performed in buffers that have been optimized, e.g., for targets (e.g., proteins, cells. etc.) that are subjects of the screens. In some embodiments, screens are performed in buffers that reduce nonspecific binding, for example, buffers comprising bovine serum albumin, bovine gamma-globulin, collagen and sheared collagen, milk proteins, randomized mixtures of synthetic peptides, lysates of bacteria or mammalian cells, lysates or protein preparations subjected to limited proteolysis, poly(deoxyinosinic-deoxycytidylic) acid, salmon sperm DNA, sheared DNA, polylysine, glycerol, trehalose, detergents such as Triton X-100, NP-40, Tween-20, Tween-80, Pleuronic F-127, octyl beta-D-glucopyranoside, etc. Additional additives are known to those skilled in the art and can be utilized in accordance with the present disclosure.

Using technologies described herein, e.g., peptide collections (libraries), screens, etc., peptide binders for diverse protein targets (e.g., beta-catenin (CTNNB1), CBL, CBLB, TEAD4, ERG) were identified.

In some embodiments, stapled peptides or collections thereof may be assessed relative to reference peptides and/or collections thereof. In some embodiments, a reference peptide is an unstapled peptide. In some embodiments, a reference peptide is an unstapled peptide of the same or a comparable sequence. In some embodiments, a peptide is a peptide comprising cysteine stapling, and a reference peptide does not have such cysteine stapling. In some embodiments, a reference peptide do not contain cysteines utilized in cysteine stapling. In some embodiments, a reference peptide does not contain a PPII region of pancreatic polypeptide, neuropeptide Y, or peptide YY which can be utilized to stabilize helical structures.

In some embodiments, a peptide collection as described herein is characterized by a certain redundancy. In some embodiments, redundancy in the context of a collection of peptides (or proteins, etc.; e.g., of a provided library) refers to the number of copies of a unique amino acid sequence within said collection of peptides. In some embodiments, a peptide collection as described herein is characterized by a redundancy of at least 10000. In some embodiments, a peptide collection as described herein is characterized by a redundancy of at least 5000. In some embodiments, a peptide collection as described herein is characterized by a redundancy of at least 1000. In some embodiments, a peptide collection as described herein is characterized by a redundancy of at least 500. In some embodiments, a peptide collection as described herein is characterized by a redundancy of at least 100. In some embodiments, a peptide collection as described herein is characterized by a redundancy of at least 50. In some embodiments, a peptide collection as described herein is characterized by a redundancy of at least 10. In some embodiments, a peptide collection as described herein is characterized by a redundancy of at least 1. In some embodiments, a preferred redundancy with respect to an actual diversity is 100-1000 phages for each unique sequence in a given screening sample, providing a good oversampling of each sequence. For example, a library of 10⁸ sequences and 1000 phages per sequence will contain 10¹¹ phage particles; in a typical screen of 48 samples, there would therefore be 4.8*10¹² total phages.

In some embodiments, a collection of peptides comprises a “spiked-in” standard. In some embodiments, a spiked-in standard is added to a collection of peptides after screening and prior to sequencing of identifying nucleic acid sequences (e.g., those encoding the peptides) of said collection of peptides. In some embodiments, a spiked-in standard is a spiked-in phage. In some embodiments, a spiked-in standard is a spiked-in nucleotide sequence. In some embodiments, a spiked-in standard serves an internal standard. In some embodiments, a spiked-in standard allows for measurement of enrichment of certain amino acid sequences.

In some embodiments, a collection of peptides can be provided and/or utilized in a phage display format. In some embodiments, the present disclosure provides a method comprising the steps of:
i) expressing in a cell a nucleic acid encoding a peptide having an amino acid sequence that: has a length within a range of a* to b* amino acid residues, includes at least one pair of cysteine residues separated by 2 to 15 amino acid residues, and wherein the nucleic acid encodes a fusion protein that is incorporated into a phage particle;
ii) isolating phage particles from the cell; and
iii) contacting phage particles with a cross-linking agent, e.g., having the structure of R^{x}-L^{s2}-R^{x} or a salt thereof or formula R-I:

R^{x}-L^{x1}-C(O)Q-L′-QC(O)-L^{x2}-R^{x} R-I,
or a salt thereof, wherein:
   a*, b*, and R^{x}, Q, and L′ are described in the present disclosure.

In some embodiments, a cross-linking agent is a reagent as described herein. In some embodiments, a cross-linking agent is a compound having the structure of R^{E}-L^{s2}-R^{E} or a salt thereof. In some embodiments, a cross-linking agent is a compound having the structure of R^{E}-L^{x1}-C(O)Q-L′-QC(O)-L^{x2}-R^{E} or a salt thereof. In some embodiments, a cross-linking agent is a compound having the structure of R^{E}-C(O)Q-L′-QC(O)-R^{E} or a salt thereof. In some embodiments, a cross-linking agent is a compound having the structure of R^{x}-L^{s2}-R^{x} or a salt thereof. In some embodiments, a cross-linking agent is a compound having the structure of R^{x}-L^{x1}-C(O)Q-L′-QC(O)-L^{x2}-R^{x} or a salt thereof.

In some embodiments, the present disclosure provides a method further comprising the steps of:
iv) contacting the collection of peptides with a target of interest so that one or more stapled peptides of the collection binds to a target of interest; and
v) determining amino acid sequences of stapled peptides that bind to a target of interest.

In some embodiments, each amino acid sequence is discretely associated with an identifier so that each amino acid sequence can be independently identified. In some embodiments, each amino acid sequence is independently associated with an identifier that comprises a nucleic acid sequence that encodes an amino acid sequence or a portion (e.g., 5, 6, 7, 8, 9, 10, 11, 12, 13, 14, 15, 16, 17, 18, 19, 20, 21, 22, 23, 24, 25 or more optionally consecutive amino acid residues, or 10%, 20%, 30%, 40%, 50%, 60%, 70%, 80%, 90%, 95% or more, of the amino acid sequence) thereof. In some embodiments, association is covalent. In some embodiments, association is through a discrete system comprising an amino acid sequence and its identifier. In some embodiments, a system is a cell, phage, etc. that comprises and/or expresses an amino acid sequence and its identifier. In some embodiments, determination of an amino acid sequence comprises determining nucleotide sequences that encode peptides of a fusion protein incorporated into a phage particle. In some embodiments, determining amino acid sequences comprises the use of high-throughput sequencing of nucleic acids encoding the amino acid sequences. In some embodiments, high-throughput sequencing comprises single-molecule real-time sequencing, ion semiconductor sequencing (e.g. Ion Torrent Sequencing), pyrosequencing, sequencing by synthesis, sequencing by ligation (e.g. SOLiD sequencing), nanopore sequencing, etc. As appreciated by those skilled in the art, various high-throughput sequencing technologies can be utilized in accordance with the present disclosure. In some embodiments, the present disclosure recognizes that technologies prior to the present disclosure can be severely and inherently limited and in many instances cannot be successfully performed when libraries containing large numbers of diverse amino acid sequences are utilized as e.g., amino acid sequences with desired properties and/or activities cannot be readily identified from background noise, e.g., due to the limited numbers of sequences that can be individually assessed. Among other things, the present disclosure demonstrates that the combination of peptide libraries and high-throughput sequencing of the corresponding encoding nucleic acid sequences are particularly powerful for screening peptide libraries comprising large numbers of diverse sequences and identifying from such libraries useful amino acid sequences, in some instances, amino acid sequences very different or totally different from natural amino acid sequences. In some embodiments, provided technologies greatly improve throughput and/or enable analysis/assessment of many candidate sequences at unprecedented level.

In some embodiments, a peptide can be identified through mass spectrometry. In some embodiments, mass spectrometry identifies peptides by their mass. In some embodiments, mass spectrometry identifies peptides by sequencing. In some embodiments, collections are designed and many or all peptides have unique mass.

In some embodiments, the present disclosure provides a method further comprising the steps of:
vi) synthesizing a new collection of peptides guided by patterns observed during the determination of amino acid sequences of stapled peptides that bind to a target of interest.

In some embodiments, the present disclosure provides a method comprising identifying or characterizing a binding agent specific for a target of interest, the method comprising steps of contacting the target of interest with members of a collection of stapled peptides; and detecting specific binding by one or more of the members to the target.

In some embodiments, a collection of peptides is a collection of polypeptides. In some embodiments, a collection of peptides is a collection of polypeptides with secondary structure. In some embodiments, a collection of peptides is a collection of polypeptides with a tertiary structure. In some embodiments, a collection of polypeptides is a collection of proteins. In some embodiments, a collection of proteins is a collection of stapled proteins.

As described in the present disclosure, in some embodiments, peptides in a library comprise an amino acid sequences, e.g., for screening. In some embodiments, such amino acid sequences comprise stapled amino acid residues. In some embodiments, such amino acid sequences have the same length, and/or have the staples at the same positions. In some embodiments, the amino acid sequences in a collection of peptides of a library comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 random positions, the amino acid residue at each of such positions can independently be any amino acid residue (e.g., natural amino acid residue). In some embodiments, there are at least 5 random positions. In some embodiments, there are at least 6 random positions. In some embodiments, there are at least 7 random positions. In some embodiments, there are at least 8 random positions. In some embodiments, there are at least 9 random positions. In some embodiments, there are at least 10 random positions. In some embodiments, there are at least 11 random positions. In some embodiments, there are at least 12 random positions. In some embodiments, there are at least 13 random positions. In some embodiments, there are at least 14 random positions. In some embodiments, there are at least 15 random positions. In some embodiments, there are at least 16 random positions. In some embodiments, there are at least 17 random positions. In some embodiments, each amino acid residue except the stapled residues is independently random. In some embodiments, at a random position there are at least 2, 3, 4, 5, 6, 7, 8, 9, 10, 11, 12, 13, 14, 15, 16, 17, 18, 19 or 20 different amino acid residues with in a collection of peptides of a library. In some embodiments, there are at least 2 different amino acid residues. In some embodiments, there are at least 3 different amino acid residues. In some embodiments, there are at least 4 different amino acid residues. In some embodiments, there are at least 5 different amino acid residues. In some embodiments, there are at least 6 different amino acid residues. In some embodiments, there are at least 7 different amino acid residues. In some embodiments, there are at least 8 different amino acid residues. In some embodiments, there are at least 9 different amino acid residues. In some embodiments, there are at least 10 different amino acid residues. In some embodiments, there are at least 15 different amino acid residues. In some embodiments, there are at least 20 different amino acid residues.

### Library Scaffold

In some embodiments, the present disclosure provides a collection of peptides of the structure:
[X⁰]ₚ₀[X¹]ₚ₁[X²]ₚ₂[X³]ₚ₃[X⁴]ₚ₄[X⁵]ₚ₅[X⁶]ₚ₆[X⁷]ₚ₇[X⁸]ₚ₈[X⁹]ₚ₉[X¹⁰]ₚ₁₀[X¹¹]ₚ₁₁[X¹²]ₚ₁₂[X¹³]ₚ₁₃-X¹⁴X¹⁵X¹⁶X¹⁷X¹⁸X¹⁹-[X²⁰]ₚ₂₀[X²¹]ₚ₂₁[X²²]ₚ₂₂[X²³]ₚ₂₃[X²⁴]ₚ₂₄[X²⁵]ₚ₂₅[X²⁶]ₚ₂₆[X²⁷]ₚ₂₇[X²⁸]ₚ₂₈[X²⁹]ₚ₂₉[X³⁰]ₚ₃₀[X³¹]ₚ₃₁[X³²]ₚ₃₂,
   wherein:
X⁰ is a scaffold constant region;
each of p0, p1, p2, p3, p4, p5, p6, p7, p8, p9, p10, p11, p12, p13, p20, p21, p22, p23, p24, p25, p26, p27, p28, p29, p30, p31, p32 is independently 0 or 1;
each of X¹ to X³² is independently an amino acid residue;
at least two of X¹ to X³² comprise side chains that are optionally linked together to form a staple.

In some embodiments, p0 is 0. In some embodiments, p0 is 1.

In some embodiments, a scaffold constant region provides a peptide sequence that is at least 50%, 60%, 70%, 80%, 90%, or 95% homologous to naturally occurring amino acid sequence. In some embodiments, the homology is at least 50%. In some embodiments, the homology is at least 60%. In some embodiments, the homology is at least 70%. In some embodiments, the homology is at least 80%. In some embodiments, the homology is at least 90%. In some embodiments, the homology is at least 95%. In some embodiments, a scaffold constant region comprises the amino acid sequence AGPAKPEAGEDASP (SEQ ID NO: 19).

### Targets of Interest

In some embodiments, the present disclosure provides reagents, peptide agents, and combinations of peptides that interact with a target of interest. In some embodiments, a target of interest is a biomolecule (e.g. protein, RNA, DNA, etc.), a tissue, or a cell. In some embodiments, a target of interest has an activity or characteristic that is associated with a disease, disorder or condition. In some embodiments, a target of interest is a protein. In some embodiments, a target of interest is a protein associated with a condition, disorder or disease, e.g., a protein associated with cancer. Many targets associated with various conditions, disorders or diseases are known in the art and can be targeted using technologies of the present disclosure.

In some embodiments, a target of interest may be an entity that occurs in a biological system or organism (e.g., a human). In some embodiments, a target of interest may have a known interaction partner. In some embodiments, a target of interest may not have any known interaction partner, or may not have any known interaction partner whose binding interaction with the target is characterized by one or more features as described herein.

In some embodiments, a target of interest may be or comprise one or more of beta-catenin and Casitas B-lineage lymphoma proto-oncogene-b (Cbl-b). In some embodiments, a target of interest is beta-catenin. In some embodiments, a target of interest is Casitas B-lineage lymphoma proto-oncogene-b (Cbl-b).

### Reagents

In some embodiments, the present disclosure provides reagents useful in the production, identification, characterization and/or use of stapled peptides (e.g., cysteine stapled peptides). In some embodiments, provided reagents are particularly useful, for example, they provide high yields and/or purities when used in combination of a biological system (e.g., a system that expresses amino acid sequences).

In some embodiments, the present disclosure provides compounds (e.g., that may be useful as reagents) having the structure of R^{E}-L^{s2}-R^{E} or a salt thereof, wherein each variable is as described in the present disclosure. In some embodiments, R^{E} is or comprises an electrophilic group. As appreciated by those skilled in the art, various electrophilic groups are known and utilized in the art and can be utilized in accordance with the present disclosure. In some embodiments, an electrophilic group reacts with —SH, e.g., —SH of an amino acid side chain, under suitable conditions (e.g., certain pH conditions utilized in the art) so that a reaction occurs between the —SH group and the electrophilic group forming a covalent bond between the —S— and the electrophilic group. In some embodiments, an electrophilic group comprises a leaving group bonded to a carbon atom, e.g., —CH₂R^{x}, wherein R^{x} is a leaving group. In some embodiments, —SH reacts with —CH₂R^{x} to form —CH₂—S—. In some embodiments, an electrophilic group is or comprises a double or triple bond. In some embodiments, a double or triple bond is bonded to one or more electron-withdrawing groups (e.g., one or more —C(O)— groups). In some embodiments, an electrophilic group is a Michael accepter. In some embodiments, an electrophilic group is or comprises

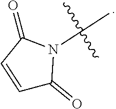 [SMILES: *N1C(=O)C=CC1=O]

In some embodiments, R^{E} is

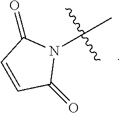 [SMILES: *N1C(=O)C=CC1=O]

In some embodiments, R^{E} is R^{x}-L^{x1}-. In some embodiments, two R^{E} in the same molecule is the same. In some embodiments, they are different. In some embodiments, useful compounds as reagents have the formula of R^{E}-L^{x1}-EWG-Q-L′-Q-EWG-L^{x2}-R^{E} or a salt thereof, wherein each EWG is independently an electron-withdrawing group moiety (e.g., being or comprising —C(O)—, —S(O)—, —S(O)₂—, etc.) and each other variable is independently as described herein. In some embodiments, useful compounds as reagents have the formula of R^{E}-L^{x1}-C(O)Q-L′-QC(O)-L^{x2}-R^{E} or a salt thereof, wherein each variable is independently as described herein. In some embodiments, useful compounds as reagents have the formula of R^{E}-L^{x1}-SO₂-Q-L′-Q-SO₂-L^{x2}-R^{E} or a salt thereof, wherein each variable is independently as described herein. In some embodiments, useful compounds as reagents have the formula of R^{E}-EWG-Q-L′-Q-EWG-R^{E} or a salt thereof, wherein each EWG is independently an electron-withdrawing group moiety (e.g., being or comprising —C(O)—, —S(O)—, —S(O)₂—, etc.) and each other variable is independently as described herein. In some embodiments, useful compounds as reagents have the formula of R^{E}—C(O)Q-L′-QC(O)-R^{E} or a salt thereof, wherein each variable is independently as described herein. In some embodiments, useful compounds as reagents have the formula of R^{E}-SO₂-Q-L′-Q-SO₂-R^{E} or a salt thereof, wherein each variable is independently as described herein. In some embodiments, useful compounds as reagents have the formula of R^{E}-Q-L′-Q-R^{E} or a salt thereof, wherein each variable is independently as described herein. In some embodiments, the present disclosure provides compounds (e.g., that may be useful as reagents) having the structure of R^{x}-L^{s2}-R^{x} or a salt thereof, wherein each variable is as described in the present disclosure. In some embodiments, the present disclosure provides compounds (e.g., that may be useful as reagents) having the formula R-I:

R^{x}-L^{x1}-C(O)Q-L′-QC(O)-L^{x2}-R^{x} R-I

or a salt thereof, wherein:
each R^{x} is independently a leaving group;
each Q is independently selected from a covalent bond, —N(R′)—, —O—, and —S—;
each of L^{x1}, L^{x2}, and L′ is independently a covalent bond, or an optionally substituted, bivalent C₁-C₂₀ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon; and

Various leaving groups are known in the art and may be utilized in accordance with the present disclosure, e.g., as embodiments for R^{x}. In some embodiments, a leaving group, e.g., R^{x}, is a halogen. In some embodiments R^{x} is —Cl. In some embodiments R^{x} is —Br. In some embodiments R^{x} is —I. In some embodiments, a leaving group is —OSO₂R (e.g., wherein R is optionally substituted alkyl (e.g., perfluoroalkyl such as —CF₃)), optionally substituted phenyl, etc.). In some embodiments, a leaving group is -OTs or -OMs. In some embodiments, R^{x} is bonded to —CH₂—. In some embodiments, R^{x} is boned to a propargyl or allylic carbon atom, or carbon bonded to an aromatic ring, e.g., a benzylic carbon atom.

In some embodiments, R^{x} groups within the same compound are the same. In some embodiments, R^{x} groups within the same compound are different.

In some embodiments, L^{x1} is an optionally substituted methylene group. In some embodiments, L^{x1} is —CH₂—. In some embodiments, L^{x2} is an optionally substituted methylene group. In some embodiments, L^{x2} is —CH₂—. In some embodiments, L^{x1} and L^{x2} are the same; in other embodiments, they are different. In some embodiments, both L^{x1} and L^{x2} are —CH₂—.

In some embodiments, Q is a covalent bond. In some embodiments, Q is selected from a covalent bond, —N(R′)—, —O—, and —S—. In some embodiments, Q is —N(R′)—, wherein R′ is described in the present disclosure. In some embodiments, Q is —O—. In some embodiments, Q is —S—. In some embodiments, each Q is a covalent bond. In some embodiments, each Q is independently selected from —N(R′)—, —O—, and —S—. In some embodiments, each Q is independently —N(R′)—, wherein R′ is described in the present disclosure. In some embodiments, each Q is independently —NH—. In some embodiments, each Q is independently —O—. In some embodiments, each Q is independently —S—.

In some embodiments, —Cy— is an optionally substituted bivalent C₃₋₂₀ cycloaliphatic ring. In some embodiments, —Cy— is an optionally substituted bivalent C₆₋₂₀ aryl ring. In some embodiments, —Cy— is an optionally substituted bivalent phenyl ring. In some embodiments, —Cy— is an optionally substituted 5-20 membered heteroaryl ring having 1-10 heteroatoms independently selected from oxygen, nitrogen, sulfur, phosphorus and silicon. In some embodiments, —Cy— is an optionally substituted 3-20 membered heterocyclyl ring having 1-10 heteroatoms independently selected from oxygen, nitrogen, sulfur, phosphorus and silicon. In some embodiments, —Cy— is monocyclic. In some embodiments, —Cy— is bicyclic. In some embodiments, —Cy— is polycyclic. In some embodiments, —Cy— comprises one or more optionally substituted heterocyclic rings, wherein each of the heterocyclic rings independently comprises one or more nitrogen atoms each of which is independently bonded to Q, or —C(O)— when Q is a covalent bond. In some embodiments, —Cy— is monocyclic. In some embodiments, —Cy— is bicyclic. In some embodiments, —Cy— is polycyclic.

In some embodiments, for a bicyclic or polycyclic ring, each of the monocyclic rings is independently a 3-10 (e.g., 3, 4, 5, 6, 7, 8, 9, or 10) membered saturated or partially unsaturated ring having 0-10 (e.g., 0, 1, 2, 3, 4, 5, 6, 7, 8, 9, or 10) heteroatoms (e.g., in some embodiments, selected from nitrogen, oxygen and sulfur) or a 5-10 (e.g., 3, 4, 5, 6, 7, 8, 9, or 10) membered aromatic ring having 0-10 (e.g., 0, 1, 2, 3, 4, 5, 6, 7, 8, 9, or 10) heteroatoms (e.g., in some embodiments, selected from nitrogen, oxygen and sulfur).

In some embodiments, L′ is or comprises —Cy—. In some embodiments, L′ is —Cy—.

In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₈ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₃ aliphatic. In some embodiments, L′ is optionally substituted bivalent C₁-C₂ aliphatic. In some embodiments, L′ is optionally substituted bivalent C₁ aliphatic. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—.

In some embodiments, L′ is optionally substituted bivalent C₆₋₂₀ aryl ring. In some embodiments, L′ is optionally substituted bivalent C₆₋₁₄ aryl ring. In some embodiments, L′ is optionally substituted bivalent C₆₋₁₀ aryl ring. In some embodiments, L′ is optionally substituted bivalent C₆ aryl ring. In some embodiments, L′ is bivalent C₆ aryl substituted with at least one electron-withdrawing group as appreciated by those skilled in the art. In some embodiments, L′ is bivalent C₆ aryl substituted with at least one halogen. In some embodiments, L′ is bivalent C₆ aryl substituted with at least two halogen. In some embodiments, L′ is bivalent C₆ aryl substituted with at least three halogen. In some embodiments, L′ is bivalent C₆ aryl substituted with four halogen. In some embodiments, L′ is bivalent C₆ aryl substituted with at least one fluorine. In some embodiments, L′ is bivalent C₆ aryl substituted with at least two fluorine. In some embodiments, L′ is bivalent C₆ aryl substituted with at least three fluorine. In some embodiments, L′ is bivalent C₆ aryl substituted with four fluorine. In some embodiments, L′ is bivalent C₆ aryl substituted with at least one chlorine. In some embodiments, L′ is bivalent C₆ aryl substituted with at least two chlorine. In some embodiments, L′ is bivalent C₆ aryl substituted with at least three chlorine. In some embodiments, L′ is bivalent C₆ aryl substituted with four chlorine. In some embodiments, L′ is bivalent C₆ aryl substituted at with least one —O(CH₂)₀₋₄CH₃. In some embodiments, L′ is bivalent C₆ aryl substituted with at least two —O(CH₂)₀₋₄CH₃. In some embodiments, L′ is bivalent C₆ aryl substituted with at least three —O(CH₂)₀₋₄CH₃. In some embodiments, L′ is bivalent C₆ aryl substituted with four —O(CH₂)₀₋₄CH₃. In some embodiments, L′ is optionally substituted

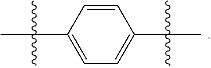 [SMILES: *c1ccc(*)cc1]

In some embodiments, L′ is

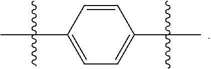 [SMILES: *c1ccc(*)cc1]

In some embodiments, L′ is substituted

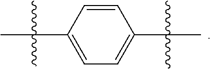 [SMILES: *c1ccc(*)cc1]

In some embodiments, L′ is di-substituted

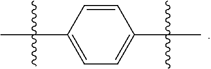 [SMILES: *c1ccc(*)cc1]

In some embodiments, L′ is 2,5-di-substituted

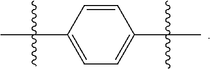 [SMILES: *c1ccc(*)cc1]

In some embodiments, L′ comprises or is bivalent 5-20 membered heteroaryl ring having 1-10 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, and sulfur. In some embodiments, L′ is bivalent 6 membered heteroaryl ring having 1-2 heteroatoms independently selected from oxygen, nitrogen, and sulfur. In some embodiments, L′ is bivalent 6 membered heteroaryl ring having 2 nitrogen.

In some embodiments, L′ is optionally substituted bivalent C₃₋₂₀ cycloaliphatic ring. In some embodiments, L′ is optionally substituted bivalent C₃₋₁₅ cycloaliphatic ring. In some embodiments, L′ is optionally substituted bivalent C₃₋₁₀ cycloaliphatic ring. In some embodiments, L′ is optionally substituted bivalent C₃₋₉ cycloaliphatic ring. In some embodiments, L′ is optionally substituted bivalent C₃₋₈ cycloaliphatic ring. In some embodiments, L′ is optionally substituted bivalent C₃₋₇ cycloaliphatic ring. In some embodiments, L′ is optionally substituted bivalent C₃₋₆ cycloaliphatic ring. In some embodiments, L′ is optionally substituted bivalent C₃₋₅ cycloaliphatic ring. In some embodiments, L′ is optionally substituted bivalent C₃₋₄ cycloaliphatic ring. In some embodiments, L′ is optionally substituted bivalent C₃ cycloaliphatic ring. In some embodiments, L′ is optionally substituted bivalent C₄ cycloaliphatic ring. In some embodiments, L′ is optionally substituted bivalent C₅ cycloaliphatic ring. In some embodiments, L′ is optionally substituted bivalent C₅ cycloalkyl ring. In some embodiments, L′ is optionally substituted bivalent C₅ cycloalkenyl ring. In some embodiments, L′ is optionally substituted bivalent C₆ cycloaliphatic ring. In some embodiments, L′ is optionally substituted bivalent C₆ cycloalkyl ring.

In some embodiments, L^{s2} comprises —N(R′)-L′-N(R′)- and L′ is a covalent bond. In some embodiments L^{s2} comprises —N(R)—N(R)—, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments L^{s2} comprises —N(R)—N(R)—, wherein:
each R is independently optionally substituted C₁₋₃₀ aliphatic; or
two or more R groups on two or more atoms are optionally and independently taken together with their intervening atoms to form an optionally substituted, 3-30 membered monocyclic ring.

In some embodiments, L^{s2} is -L^{x1}-C(O)Q-L′-QC(O)-L^{x1}-, wherein each variable is independently as described in the present disclosure. In some embodiments, L^{s2} is —CH₂C(O)Q-L′-QC(O)CH₂—, wherein each —CH₂— is independently and optionally substituted. In some embodiments, L^{s2} is —CH₂C(O)Q-L′-QC(O)CH₂—. In some embodiments, a provided compound, e.g., a compound of R^{x}-L^{s2}-R^{x}, is selected from the group consisting of: Table 1.

TABLE 1

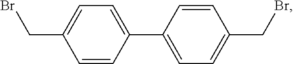 [SMILES: BrCc1ccc(-c2ccc(CBr)cc2)cc1]

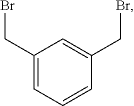

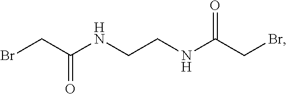 [SMILES: O=C(CBr)NCCNC(=O)CBr]

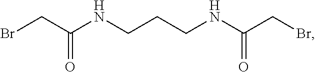 [SMILES: O=C(CBr)NCCCNC(=O)CBr]

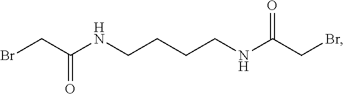 [SMILES: O=C(CBr)NCCCCNC(=O)CBr]

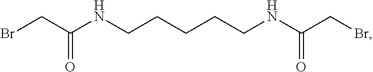 [SMILES: O=C(CBr)NCCCCCNC(=O)CBr]

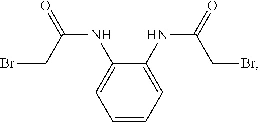 [SMILES: O=C(CBr)Nc1ccccc1NC(=O)CBr]

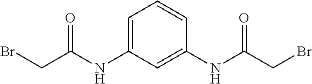 [SMILES: O=C(CBr)Nc1cccc(NC(=O)CBr)c1]

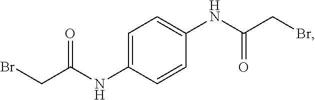 [SMILES: O=C(CBr)Nc1ccc(NC(=O)CBr)cc1]

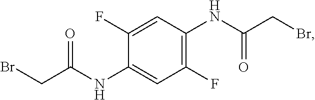 [SMILES: O=C(CBr)Nc1cc(F)c(NC(=O)CBr)cc1F]

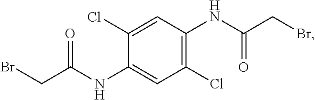 [SMILES: O=C(CBr)Nc1cc(Cl)c(NC(=O)CBr)cc1Cl]

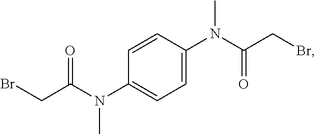 [SMILES: CN(C(=O)CBr)c1ccc(N(C)C(=O)CBr)cc1]

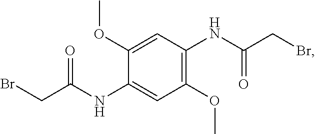 [SMILES: COc1cc(NC(=O)CBr)c(OC)cc1NC(=O)CBr]

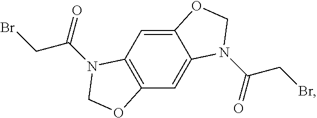 [SMILES: O=C(CBr)N1COc2cc3c(cc21)OCN3C(=O)CBr]

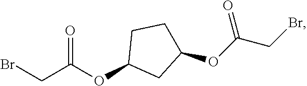 [SMILES: O=C(CBr)O[C@@H]1CC[C@H](OC(=O)CBr)C1]

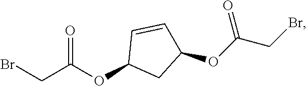 [SMILES: O=C(CBr)O[C@@H]1C=C[C@H](OC(=O)CBr)C1]

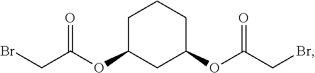 [SMILES: O=C(CBr)O[C@@H]1CCC[C@H](OC(=O)CBr)C1]

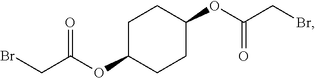 [SMILES: O=C(CBr)O[C@H]1CC[C@@H](OC(=O)CBr)CC1]

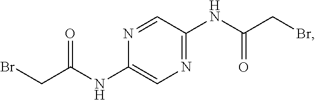 [SMILES: O=C(CBr)Nc1cnc(NC(=O)CBr)cn1]

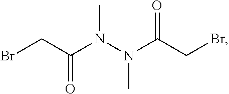

and

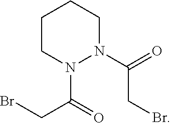 [SMILES: O=C(CBr)N1CCCCN1C(=O)CBr]

In some embodiments, a provided compound, e.g., a compound of R^{x}-L^{s2}-R^{x}, has the structure of R^{x}-L^{x1}-C(O)Q-L′-QC(O)-L^{x2}-R^{x} or R^{x}—CH₂C(O)Q-L′-QC(O)CH₂-R^{x} and is selected from

TABLE 2

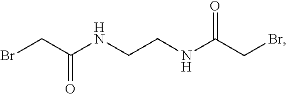 [SMILES: O=C(CBr)NCCNC(=O)CBr]

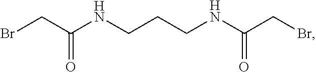 [SMILES: O=C(CBr)NCCCNC(=O)CBr]

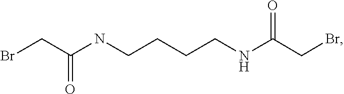

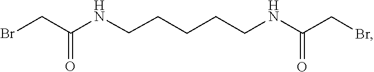 [SMILES: O=C(CBr)NCCCCCNC(=O)CBr]

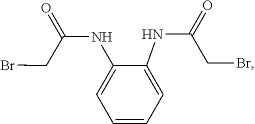 [SMILES: O=C(CBr)Nc1ccccc1NC(=O)CBr]

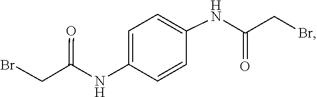 [SMILES: O=C(CBr)Nc1ccc(NC(=O)CBr)cc1]

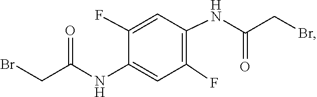 [SMILES: O=C(CBr)Nc1cc(F)c(NC(=O)CBr)cc1F]

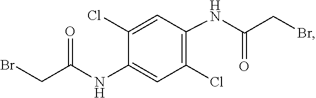 [SMILES: O=C(CBr)Nc1cc(Cl)c(NC(=O)CBr)cc1Cl]

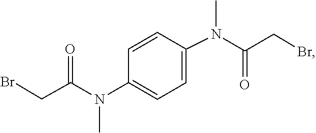 [SMILES: CN(C(=O)CBr)c1ccc(N(C)C(=O)CBr)cc1]

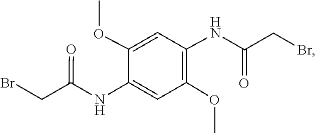 [SMILES: COc1cc(NC(=O)CBr)c(OC)cc1NC(=O)CBr]

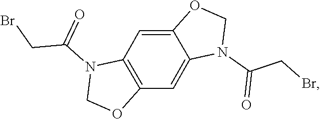 [SMILES: O=C(CBr)N1COc2cc3c(cc21)OCN3C(=O)CBr]

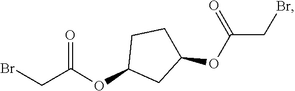 [SMILES: O=C(CBr)O[C@@H]1CC[C@H](OC(=O)CBr)C1]

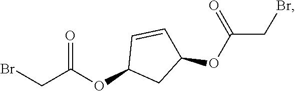 [SMILES: O=C(CBr)O[C@@H]1C=C[C@H](OC(=O)CBr)C1]

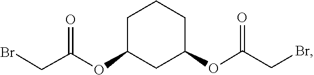 [SMILES: O=C(CBr)O[C@@H]1CCC[C@H](OC(=O)CBr)C1]

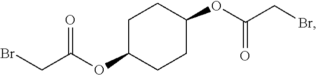 [SMILES: O=C(CBr)O[C@H]1CC[C@@H](OC(=O)CBr)CC1]

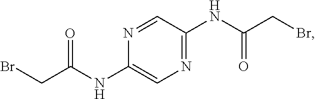 [SMILES: O=C(CBr)Nc1cnc(NC(=O)CBr)cn1]

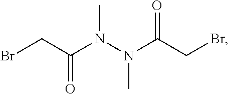

and

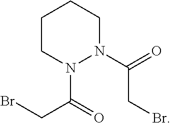 [SMILES: O=C(CBr)N1CCCCN1C(=O)CBr]

In some embodiments, a compound is not

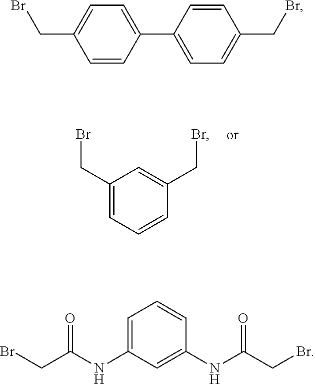 [SMILES: BrCc1ccc(-c2ccc(CBr)cc2)cc1 ; BrCc1cccc(CBr)c1 ; O=C(CBr)Nc1cccc(NC(=O)CBr)c1]

In some embodiments, a compound is not

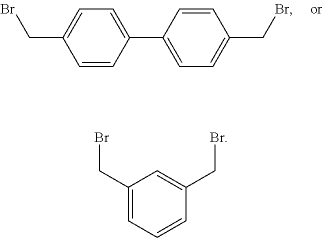 [SMILES: BrCc1ccc(-c2ccc(CBr)cc2)cc1 ; BrCc1cccc(CBr)c1]

In some embodiments, a compound is not

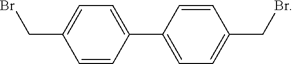 [SMILES: BrCc1ccc(-c2ccc(CBr)cc2)cc1]

In some embodiments, a compound is not

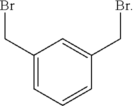

In some embodiments, a compound is not

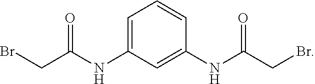 [SMILES: O=C(CBr)Nc1cccc(NC(=O)CBr)c1]

In some embodiments, L^{x2} or —CH₂C(O)Q-L′-QC(O)CH₂— is as described in such compounds, wherein R^{x} is —Br. In some embodiments, L^{s} or L^{s2} is

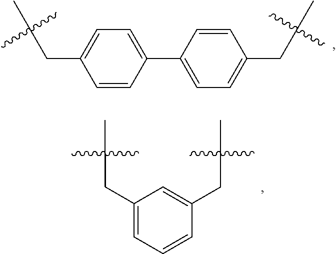 [SMILES: *Cc1ccc(-c2ccc(C*)cc2)cc1 ; *Cc1cccc(C*)c1]

or

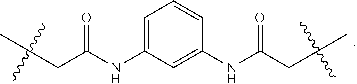 [SMILES: *CC(=O)Nc1cccc(NC(=O)C*)c1]

In some embodiments, L^{s} or L^{s2} is not

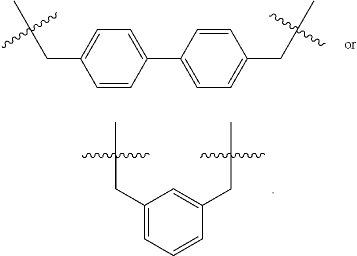 [SMILES: *Cc1ccc(-c2ccc(C*)cc2)cc1 ; *Cc1cccc(C*)c1]

In some embodiments, L^{s} or L^{s2} is not

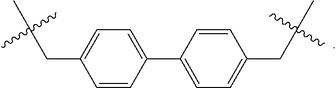 [SMILES: *Cc1ccc(-c2ccc(C*)cc2)cc1]

In some embodiments, L^{s} or L^{s2} is not

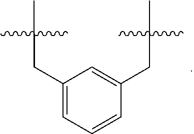 [SMILES: *Cc1cccc(C*)c1]

In some embodiments, L^{s} or L^{s2} is

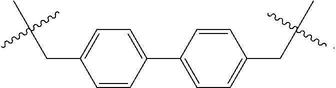 [SMILES: *Cc1ccc(-c2ccc(C*)cc2)cc1]

In some embodiments, L^{s} or L^{s2} is

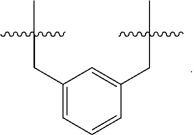 [SMILES: *Cc1cccc(C*)c1]

In some embodiments, L^{s} or L^{s2} is substituted

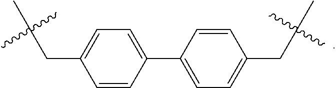 [SMILES: *Cc1ccc(-c2ccc(C*)cc2)cc1]

In some embodiments, L^{s} or L^{s2} is substituted

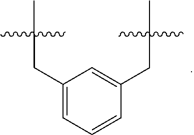 [SMILES: *Cc1cccc(C*)c1]

In some embodiments, L^{s} or L^{s2} or -L^{x1}-C(O)Q-L′-QC(O)-L^{x1}- or —CH₂C(O)Q-L′-QC(O)CH₂— is

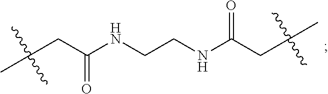 [SMILES: *CC(=O)NCCNC(=O)C*]

in some embodiments, it is substituted

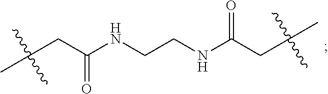 [SMILES: *CC(=O)NCCNC(=O)C*]

in some embodiments, it is

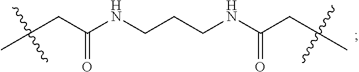 [SMILES: *CC(=O)NCCCNC(=O)C*]

in some embodiments, it is substituted

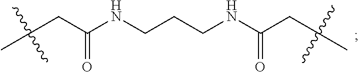 [SMILES: *CC(=O)NCCCNC(=O)C*]

in some embodiments, it is

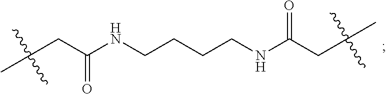 [SMILES: *CC(=O)NCCCCNC(=O)C*]

in some embodiments, it is substituted

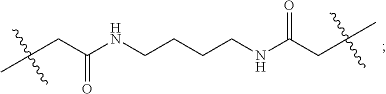 [SMILES: *CC(=O)NCCCCNC(=O)C*]

in some embodiments, it is

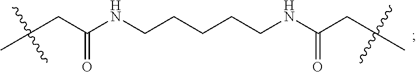 [SMILES: *CC(=O)NCCCCCNC(=O)C*]

in some embodiments, it is substituted

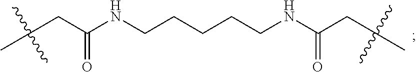 [SMILES: *CC(=O)NCCCCCNC(=O)C*]

in some embodiments, it is optionally substituted

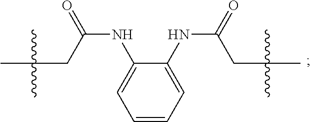 [SMILES: *CC(=O)Nc1ccccc1NC(=O)C*]

in some embodiments, it is substituted

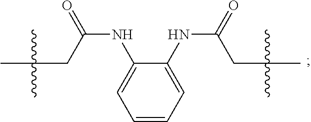 [SMILES: *CC(=O)Nc1ccccc1NC(=O)C*]

in some embodiments, it is

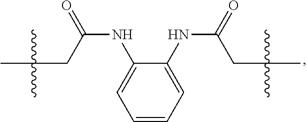 [SMILES: *CC(=O)Nc1ccccc1NC(=O)C*]

wherein the bivalent phenyl ring is optionally substituted; in some embodiments, it is

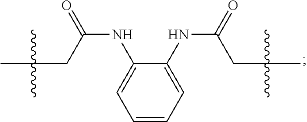 [SMILES: *CC(=O)Nc1ccccc1NC(=O)C*]

in some embodiments, it is optionally substituted

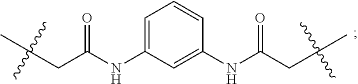 [SMILES: *CC(=O)Nc1cccc(NC(=O)C*)c1]

in some embodiments, it is

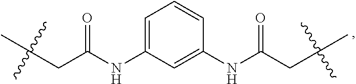 [SMILES: *CC(=O)Nc1cccc(NC(=O)C*)c1]

wherein the bivalent phenyl ring is optionally substituted; in some embodiments, it is not

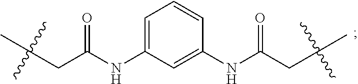 [SMILES: *CC(=O)Nc1cccc(NC(=O)C*)c1]

in some embodiments, it is

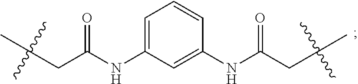 [SMILES: *CC(=O)Nc1cccc(NC(=O)C*)c1]

in some embodiments, it is substituted

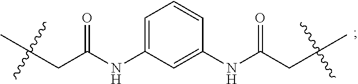 [SMILES: *CC(=O)Nc1cccc(NC(=O)C*)c1]

in some embodiments, it is optionally substituted

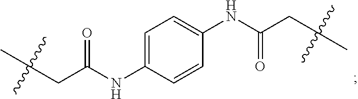 [SMILES: *CC(=O)Nc1ccc(NC(=O)C*)cc1]

in some embodiments, it is

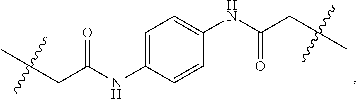 [SMILES: *CC(=O)Nc1ccc(NC(=O)C*)cc1]

wherein the bivalent phenyl ring is optionally substituted; in some embodiments, it is

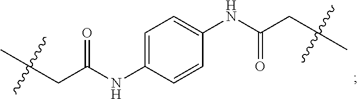 [SMILES: *CC(=O)Nc1ccc(NC(=O)C*)cc1]

in some embodiments, it is substituted

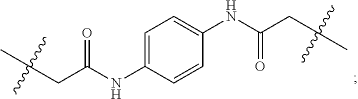 [SMILES: *CC(=O)Nc1ccc(NC(=O)C*)cc1]

in some embodiments, it is

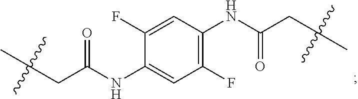 [SMILES: *CC(=O)Nc1cc(F)c(NC(=O)C*)cc1F]

in some embodiments, it is

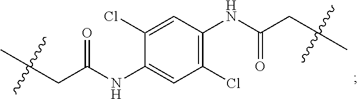 [SMILES: *CC(=O)Nc1cc(Cl)c(NC(=O)C*)cc1Cl]

in some embodiments, it is

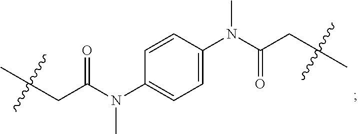 [SMILES: *CC(=O)N(C)c1ccc(N(C)C(=O)C*)cc1]

in some embodiments, it is

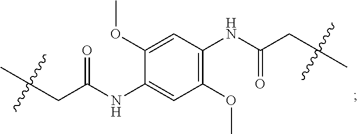 [SMILES: *CC(=O)Nc1cc(OC)c(NC(=O)C*)cc1OC]

in some embodiments, it is

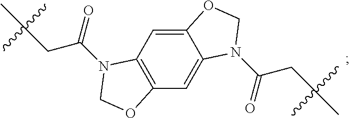 [SMILES: *CC(=O)N1COc2cc3c(cc21)OCN3C(=O)C*]

in some embodiments, it is substituted

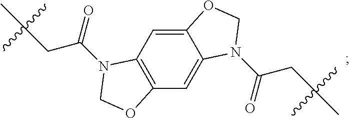 [SMILES: *CC(=O)N1COc2cc3c(cc21)OCN3C(=O)C*]

in some embodiments, it is

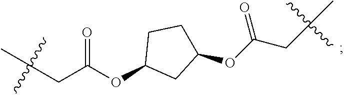 [SMILES: *CC(=O)O[C@@H]1CC[C@H](OC(=O)C*)C1]

in some embodiments, it is substituted

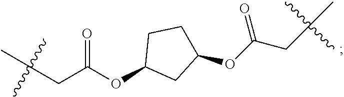 [SMILES: *CC(=O)O[C@@H]1CC[C@H](OC(=O)C*)C1]

in some embodiments, it is

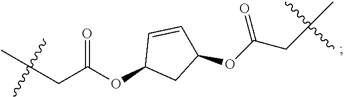 [SMILES: *CC(=O)O[C@@H]1C=C[C@H](OC(=O)C*)C1]

in some embodiments, it is substituted

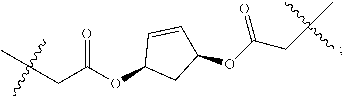 [SMILES: *CC(=O)O[C@@H]1C=C[C@H](OC(=O)C*)C1]

in some embodiments, it is

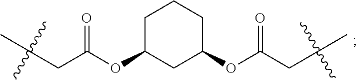 [SMILES: *CC(=O)O[C@@H]1CCC[C@H](OC(=O)C*)C1]

in some embodiments, it is substituted

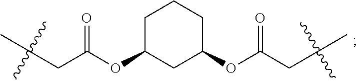 [SMILES: *CC(=O)O[C@@H]1CCC[C@H](OC(=O)C*)C1]

in some embodiments, it is

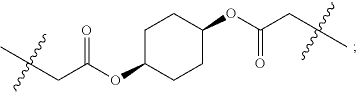 [SMILES: *CC(=O)O[C@H]1CC[C@@H](OC(=O)C*)CC1]

in some embodiments, it is substituted

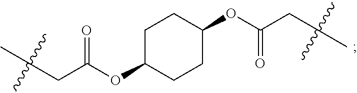 [SMILES: *CC(=O)O[C@H]1CC[C@@H](OC(=O)C*)CC1]

in some embodiments, it is

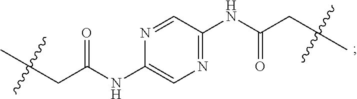 [SMILES: *CC(=O)Nc1cnc(NC(=O)C*)cn1]

in some embodiments, it is substituted

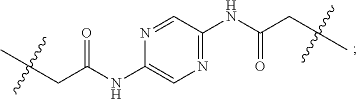 [SMILES: *CC(=O)Nc1cnc(NC(=O)C*)cn1]

in some embodiments, it is

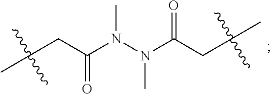 [SMILES: *CC(=O)N(C)N(C)C(=O)C*]

in some embodiments, it is substituted

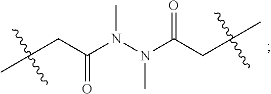 [SMILES: *CC(=O)N(C)N(C)C(=O)C*]

in some embodiments, it is

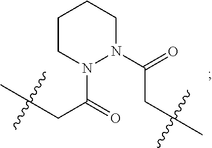 [SMILES: *CC(=O)N1CCCCN1C(=O)C*]

in some embodiments, it is substituted

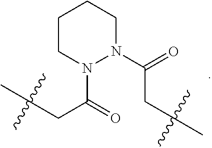 [SMILES: *CC(=O)N1CCCCN1C(=O)C*]

In some embodiments, a useful compound is selected from:

TABLE 3

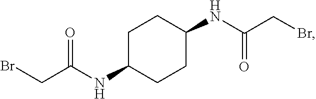 [SMILES: O=C(CBr)N[C@H]1CC[C@@H](NC(=O)CBr)CC1]

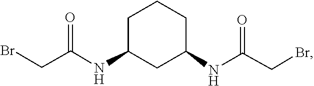 [SMILES: O=C(CBr)N[C@@H]1CCC[C@H](NC(=O)CBr)C1]

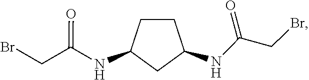 [SMILES: O=C(CBr)N[C@@H]1CC[C@H](NC(=O)CBr)C1]

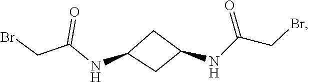 [SMILES: O=C(CBr)N[C@H]1C[C@@H](NC(=O)CBr)C1]

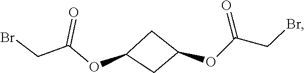 [SMILES: O=C(CBr)O[C@H]1C[C@@H](OC(=O)CBr)C1]

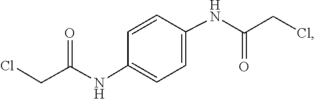 [SMILES: O=C(CCl)Nc1ccc(NC(=O)CCl)cc1]

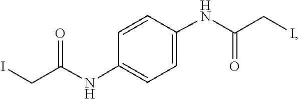 [SMILES: O=C(CI)Nc1ccc(NC(=O)CI)cc1]

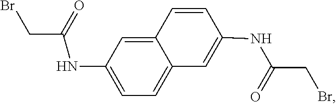 [SMILES: O=C(CBr)Nc1ccc2cc(NC(=O)CBr)ccc2c1]

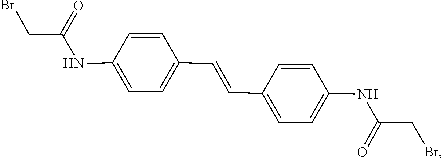 [SMILES: O=C(CBr)Nc1ccc(/C=C/c2ccc(NC(=O)CBr)cc2)cc1]

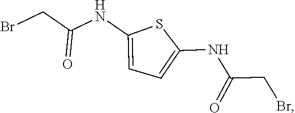 [SMILES: O=C(CBr)Nc1ccc(NC(=O)CBr)s1]

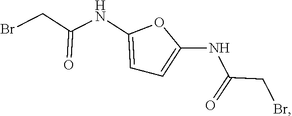 [SMILES: O=C(CBr)Nc1ccc(NC(=O)CBr)o1]

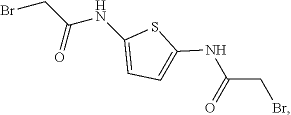 [SMILES: O=C(CBr)Nc1ccc(NC(=O)CBr)s1]

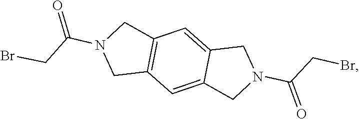 [SMILES: O=C(CBr)N1Cc2cc3c(cc2C1)CN(C(=O)CBr)C3]

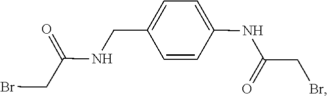 [SMILES: O=C(CBr)NCc1ccc(NC(=O)CBr)cc1]

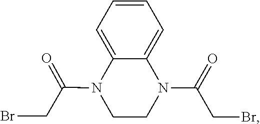 [SMILES: O=C(CBr)N1CCN(C(=O)CBr)c2ccccc21]

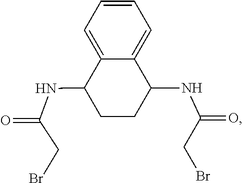 [SMILES: O=C(CBr)NC1CCC(NC(=O)CBr)c2ccccc21]

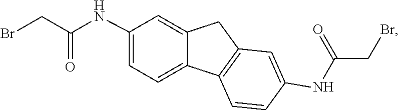 [SMILES: O=C(CBr)Nc1ccc2c(c1)Cc1cc(NC(=O)CBr)ccc1-2]

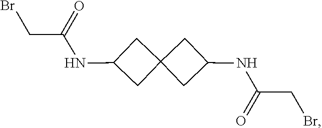 [SMILES: O=C(CBr)NC1CC2(C1)CC(NC(=O)CBr)C2]

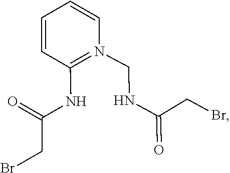

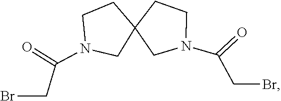 [SMILES: O=C(CBr)N1CCC2(CCN(C(=O)CBr)C2)C1]

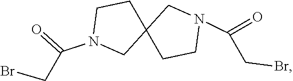 [SMILES: O=C(CBr)N1CCC2(CCN(C(=O)CBr)C2)C1]

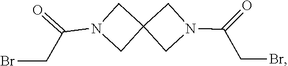 [SMILES: O=C(CBr)N1CC2(C1)CN(C(=O)CBr)C2]

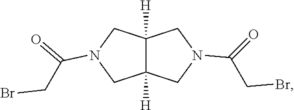 [SMILES: O=C(CBr)N1C[C@H]2CN(C(=O)CBr)C[C@H]2C1]

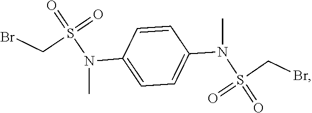 [SMILES: CN(c1ccc(N(C)S(=O)(=O)CBr)cc1)S(=O)(=O)CBr]

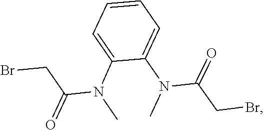 [SMILES: CN(C(=O)CBr)c1ccccc1N(C)C(=O)CBr]

and

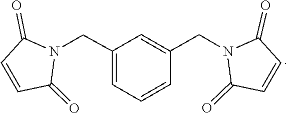 [SMILES: O=C1C=CC(=O)N1Cc1cccc(CN2C(=O)C=CC2=O)c1]

In some embodiments, L^{s} or L^{s2} or -L^{s1}-C(O)Q-L′-QC(O)-L^{x1}- or —CH₂C(O)Q-L′-QC(O)CH₂— comprise an optionally substituted saturated or partially unsaturated 3-20 (e.g., 3, 4, 5, 6, 7, 8, 9, 10, 11, 12, 13, 14, 15, 16, 17, 18, 19, or 20) membered monocyclic, bicyclic or polycyclic ring having 0-10 (0, 1, 2, 3, 4, 5, 6, 7, 8, 9, or 10) heteroatoms (e.g., in some embodiments, selected from nitrogen, oxygen and sulfur); in some embodiments, it comprises an optionally substituted 3-20 (e.g., 3, 4, 5, 6, 7, 8, 9, 10, 11, 12, 13, 14, 15, 16, 17, 18, 19, or 20) membered cycloaliphatic ring; in some embodiments, it is

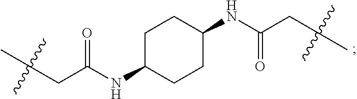 [SMILES: *CC(=O)N[C@H]1CC[C@@H](NC(=O)C*)CC1]

in some embodiments, it is substituted

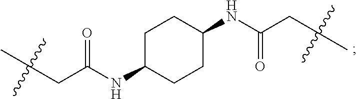 [SMILES: *CC(=O)N[C@H]1CC[C@@H](NC(=O)C*)CC1]

in some embodiments, it is

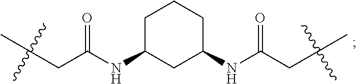 [SMILES: *CC(=O)N[C@@H]1CCC[C@H](NC(=O)C*)C1]

in some embodiments, it is substituted

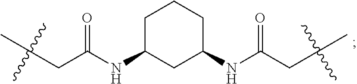 [SMILES: *CC(=O)N[C@@H]1CCC[C@H](NC(=O)C*)C1]

in some embodiments, it is

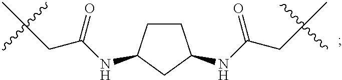 [SMILES: *CC(=O)N[C@@H]1CC[C@H](NC(=O)C*)C1]

in some embodiments, it is substituted

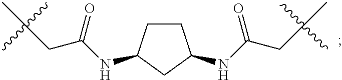 [SMILES: *CC(=O)N[C@@H]1CC[C@H](NC(=O)C*)C1]

in some embodiments, it is

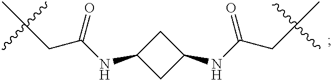 [SMILES: *CC(=O)N[C@H]1C[C@@H](NC(=O)C*)C1]

in some embodiments, it is substituted

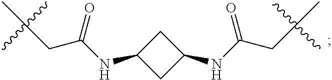 [SMILES: *CC(=O)N[C@H]1C[C@@H](NC(=O)C*)C1]

in some embodiments, it is

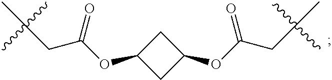 [SMILES: *CC(=O)O[C@H]1C[C@@H](OC(=O)C*)C1]

in some embodiments, it is substituted

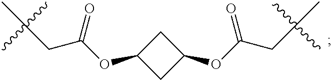 [SMILES: *CC(=O)O[C@H]1C[C@@H](OC(=O)C*)C1]

in some embodiments, it comprises an optionally substituted 6-20 (e.g., 6, 10, or 14) membered monocyclic, bicyclic or polycyclic aryl ring; in some embodiments, it is optionally substituted

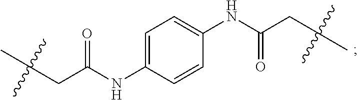 [SMILES: *CC(=O)Nc1ccc(NC(=O)C*)cc1]

in some embodiments, it is

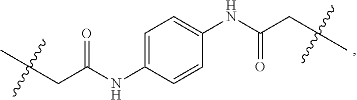 [SMILES: *CC(=O)Nc1ccc(NC(=O)C*)cc1]

wherein the phenyl ring is optionally substituted; in some embodiments, it is

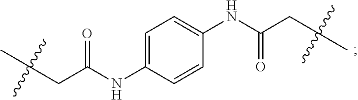 [SMILES: *CC(=O)Nc1ccc(NC(=O)C*)cc1]

in some embodiments, it is substituted

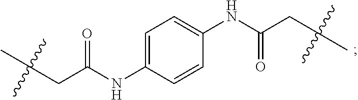 [SMILES: *CC(=O)Nc1ccc(NC(=O)C*)cc1]

in some embodiments, it is

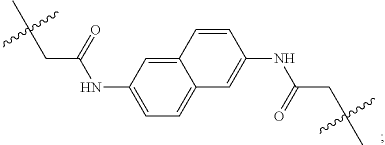 [SMILES: *CC(=O)Nc1ccc2cc(NC(=O)C*)ccc2c1]

in some embodiments, it is substituted

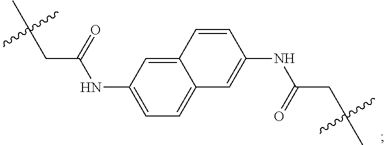 [SMILES: *CC(=O)Nc1ccc2cc(NC(=O)C*)ccc2c1]

in some embodiments, it is

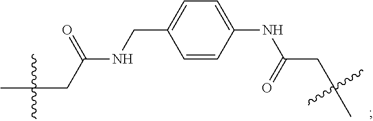 [SMILES: *CC(=O)NCc1ccc(NC(=O)C*)cc1]

in some embodiments, it is substituted

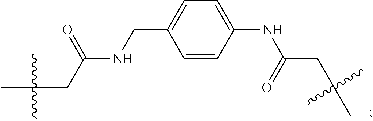 [SMILES: *CC(=O)NCc1ccc(NC(=O)C*)cc1]

in some embodiments, it is

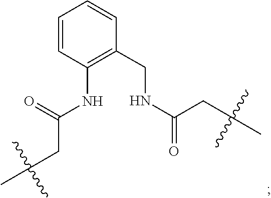 [SMILES: *CC(=O)NCc1ccccc1NC(=O)C*]

in some embodiments, it is substituted

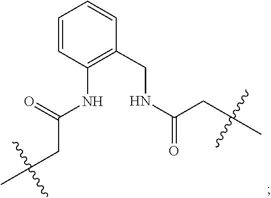 [SMILES: *CC(=O)NCc1ccccc1NC(=O)C*]

in some embodiments, it is

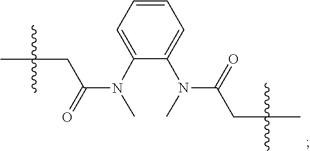 [SMILES: *CC(=O)N(C)c1ccccc1N(C)C(=O)C*]

in some embodiments, it is substituted

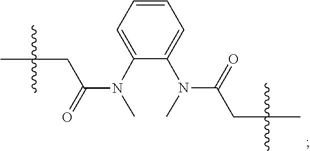 [SMILES: *CC(=O)N(C)c1ccccc1N(C)C(=O)C*]

in some embodiments, it comprises an optionally substituted 5-20 (e.g., 5, 6, 7, 8, 9, 10, 11, 12, 13, 14, 15, 16, 17, 18, 19, or 20) membered monocyclic, bicyclic or polycyclic heteroaryl ring having 0-10 (e.g., 0, 1, 2, 3, 4, 5, 6, 7, 8, 9, or 10) heteroatoms (e.g., in some embodiments, selected from nitrogen, oxygen and sulfur); in some embodiments, it is

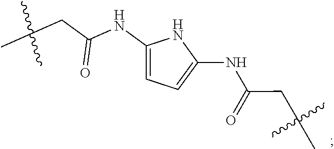 [SMILES: *CC(=O)Nc1ccc(NC(=O)C*)[nH]1]

in some embodiments, it is substituted

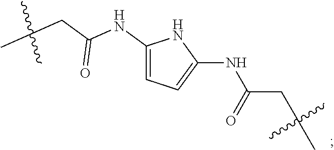 [SMILES: *CC(=O)Nc1ccc(NC(=O)C*)[nH]1]

in some embodiments, it is

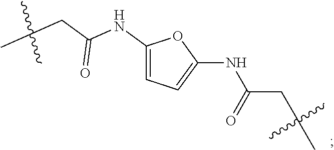 [SMILES: *CC(=O)Nc1ccc(NC(=O)C*)o1]

in some embodiments, it is substituted

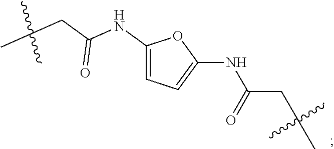 [SMILES: *CC(=O)Nc1ccc(NC(=O)C*)o1]

in some embodiments, it is

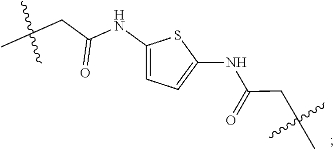 [SMILES: *CC(=O)Nc1ccc(NC(=O)C*)s1]

in some embodiments, it is substituted

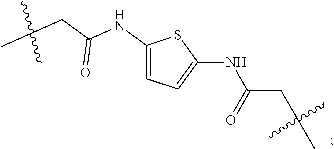 [SMILES: *CC(=O)Nc1ccc(NC(=O)C*)s1]

in some embodiments, it comprises two or more optionally substituted rings, each of which is independently a 3-10 (e.g., 3, 4, 5, 6, 7, 8, 9, or 10) membered saturated or partially unsaturated ring or 5-10 membered aromatic ring, and each of which independently has 0-10 (e.g., 0, 1, 2, 3, 4, 5, 6, 7, 8, 9, or 10) heteroatoms (e.g., in some embodiments, selected from nitrogen, oxygen and sulfur); in some embodiments, it is

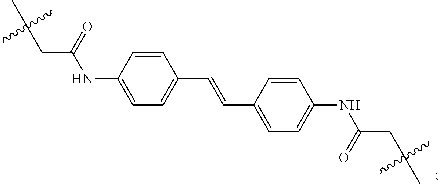 [SMILES: *CC(=O)Nc1ccc(/C=C/c2ccc(NC(=O)C*)cc2)cc1]

in some embodiments, it is substituted

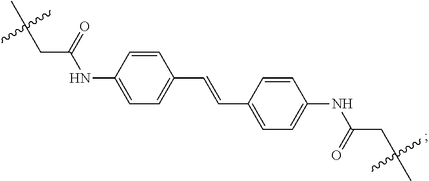 [SMILES: *CC(=O)Nc1ccc(/C=C/c2ccc(NC(=O)C*)cc2)cc1]

in some embodiments, it comprises an optionally substituted 5-20 (e.g., 5, 6, 7, 8, 9, 10, 11, 12, 13, 14, 15, 16, 17, 18, 19, or 20) membered bicyclic or polycyclic ring having 0-10 (e.g., 0, 1, 2, 3, 4, 5, 6, 7, 8, 9, or 10) heteroatoms (e.g., in some embodiments, selected from nitrogen, oxygen and sulfur), wherein each of the monocyclic rings is independently a 3-10 (e.g., 3, 4, 5, 6, 7, 8, 9, or 10) membered saturated or partially unsaturated ring having 0-10 (e.g., 0, 1, 2, 3, 4, 5, 6, 7, 8, 9, or 10) heteroatoms (e.g., in some embodiments, selected from nitrogen, oxygen and sulfur) or a 5-10 (e.g., 3, 4, 5, 6, 7, 8, 9, or 10) membered aromatic ring having 0-10 (e.g., 0, 1, 2, 3, 4, 5, 6, 7, 8, 9, or 10) heteroatoms (e.g., in some embodiments, selected from nitrogen, oxygen and sulfur); in some embodiments, it is

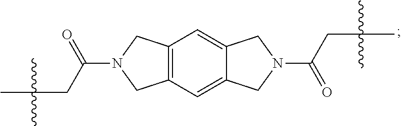 [SMILES: *CC(=O)N1Cc2cc3c(cc2C1)CN(C(=O)C*)C3]

in some embodiments, it is substituted

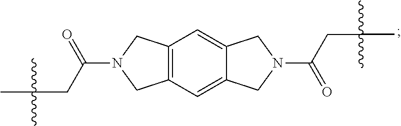 [SMILES: *CC(=O)N1Cc2cc3c(cc2C1)CN(C(=O)C*)C3]

in some embodiments, it is

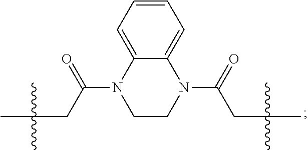 [SMILES: *CC(=O)N1CCN(C(=O)C*)c2ccccc21]

in some embodiments, it is substituted

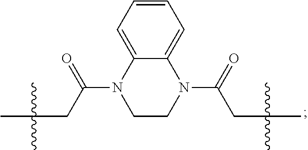 [SMILES: *CC(=O)N1CCN(C(=O)C*)c2ccccc21]

in some embodiments, it is

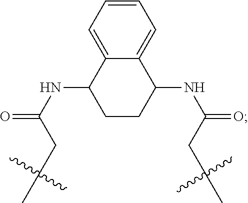 [SMILES: *CC(=O)NC1CCC(NC(=O)C*)c2ccccc21]

in some embodiments, it is substituted

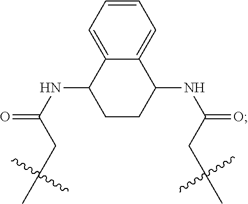 [SMILES: *CC(=O)NC1CCC(NC(=O)C*)c2ccccc21]

in some embodiments, it is

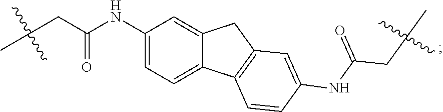 [SMILES: *CC(=O)Nc1ccc2c(c1)Cc1cc(NC(=O)C*)ccc1-2]

in some embodiments, it is substituted

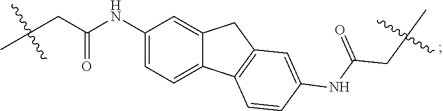 [SMILES: *CC(=O)Nc1ccc2c(c1)Cc1cc(NC(=O)C*)ccc1-2]

in some embodiments, it is

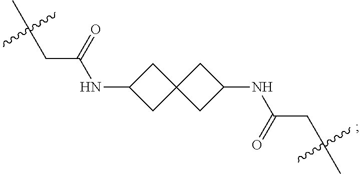 [SMILES: *CC(=O)NC1CC2(C1)CC(NC(=O)C*)C2]

in some embodiments, it is substituted

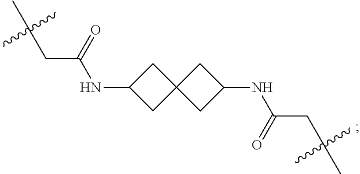 [SMILES: *CC(=O)NC1CC2(C1)CC(NC(=O)C*)C2]

in some embodiments, it is

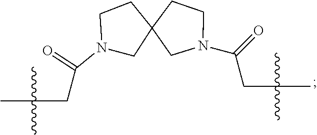 [SMILES: *CC(=O)N1CCC2(CCN(C(=O)C*)C2)C1]

in some embodiments, it is substituted

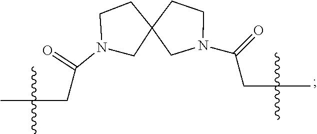 [SMILES: *CC(=O)N1CCC2(CCN(C(=O)C*)C2)C1]

in some embodiments, it is

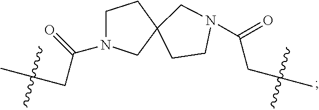 [SMILES: *CC(=O)N1CCC2(CCN(C(=O)C*)C2)C1]

in some embodiments, it is substituted

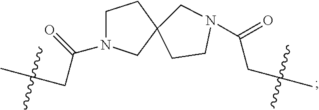 [SMILES: *CC(=O)N1CCC2(CCN(C(=O)C*)C2)C1]

in some embodiments, it is

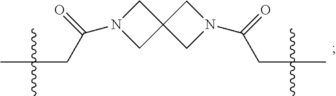 [SMILES: *CC(=O)N1CC2(C1)CN(C(=O)C*)C2]

in some embodiments, it is substituted

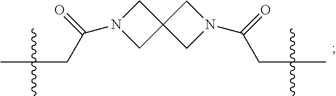 [SMILES: *CC(=O)N1CC2(C1)CN(C(=O)C*)C2]

in some embodiments, it is

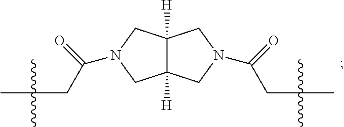 [SMILES: *CC(=O)N1C[C@H]2CN(C(=O)C*)C[C@H]2C1]

in some embodiments, it is substituted

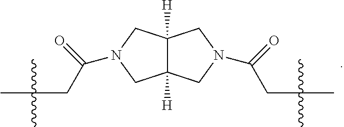 [SMILES: *CC(=O)N1C[C@H]2CN(C(=O)C*)C[C@H]2C1]

In some embodiments, L^{s} or L^{s2} is

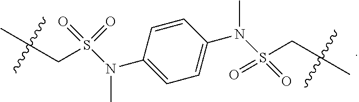 [SMILES: *CS(=O)(=O)N(C)c1ccc(N(C)S(=O)(=O)C*)cc1]

In some embodiments, L^{s} or L^{s2} is substituted

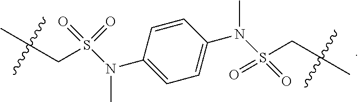 [SMILES: *CS(=O)(=O)N(C)c1ccc(N(C)S(=O)(=O)C*)cc1]

In some embodiments, L^{s} or L^{s2} is

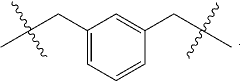 [SMILES: *Cc1cccc(C*)c1]

In some embodiments, L^{s} or L^{s2} is substituted

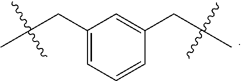 [SMILES: *Cc1cccc(C*)c1]

As described herein, rings can be of various sizes. In some embodiments, non-aromatic rings are 3-20 membered. In some embodiments, aromatic rings are 5-20 membered. In some embodiments, a ring is 3-10 membered. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. In some embodiments, a ring is 11-membered. In some embodiments, a ring is 12-membered. In some embodiments, a ring is 13-membered. In some embodiments, a ring is 14-membered. In some embodiments, a ring is 15-membered. Rings may also have various numbers and types of heteroatoms. In some embodiments, a ring has 0-10 heteroatoms. In some embodiments, a ring has 0 heteroatom. In some embodiments, a ring has 1 heteroatom. In some embodiments, a ring has 2 heteroatoms. In some embodiments, a ring has 3 heteroatoms. In some embodiments, a ring has 4 heteroatoms. In some embodiments, a ring has 5 heteroatoms. In some embodiments, a ring has 6 heteroatoms. In some embodiments, a ring has 7 heteroatoms. In some embodiments, a ring has 8 heteroatoms. In some embodiments, a ring has 9 heteroatoms. In some embodiments, a ring has 10 heteroatoms. In some embodiments, a heteroatom is selected from nitrogen, oxygen and sulfur. In some embodiments, at least one heteroatom is nitrogen. In some embodiments, at least one heteroatom is oxygen. In some embodiments, at least one heteroatom is sulfur. In some embodiments, a ring is saturated. In some embodiments, a ring is partially unsaturated. In some embodiments, a ring is aromatic. In some embodiments, a ring is a cycloaliphatic ring. In some embodiments, a ring is a cycloalkyl ring. In some embodiments, a ring is a heteroaliphatic ring. In some embodiments, a ring is a heterocyclyl ring. In some embodiments, a ring is a heterocycloalkyl ring. In some embodiments, a ring is an aryl ring. In some embodiments, a ring is a heteroaryl ring. As appreciated by those skilled in the art, rings are independently and optionally substituted—either substituted or unsubstituted.

Many additional technologies are useful for preparing stapled peptides in accordance with the present disclosure. For example, in some embodiments, amino acid residues having side chains comprising double or triple bonds and optionally various heteroatoms may be utilized to construct various types of staples, e.g., hydrocarbon staples, amino- or carbamate-containing staples, etc.

As those skilled in the art will appreciate, after stapling using reagents described herein, portions of reagents' structures are incorporated into staples in the products. Thus, various embodiments of L^{s}, L^{s2}, -L^{x1}-C(O)Q-L′-QC(O)-L^{x1}-, —CH₂C(O)Q-L′-QC(O)CH₂—, L^{x1}, Q, L′, etc. described for reagents can be utilized in staples and vice versa.

### Stapled Peptides

Stapled peptides as described herein are peptides in which two or more amino acids of a peptide chain are linked through bonding of two peptide backbone atoms of the amino acid residues and, as is understood by those skilled in the art, the resulting linker is not through the peptide backbone between the linked amino acid residues. In some embodiments, a stapled peptide comprises a staple as described herein. A staple as described herein is a linker that can link one amino acid residue to another amino acid residue through bonding two peptide backbone atoms of the amino acid residues and, as is understood by those skilled in the art, the resulting bond is not through the peptide backbone between the linked amino acid residues. In some embodiments, a staple bonds to the peptide backbone by replacing one or more hydrogen and/or substituents (e.g., side chains, O, S, etc.) on peptide backbone atoms (e.g., C, N, etc.).

As will be appreciated by those of ordinary skill in the art, a variety of peptide stapling technologies are available, including both hydrocarbon-stapling and non-hydrocarbon-stapling technologies. Certain technologies are described in U.S. Ser. No. 11/198,713, US 20210179665, WO 2021119537, WO 2021188659, WO 2022020651, or WO 2022020652, the entirety of each of which is incorporated herein by reference, and can be utilized in accordance with the present disclosure.

In some embodiments, a staple as described herein is a hydrocarbon staple. In some embodiments, a staple as described herein is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is a comprises at least one sulfur atom derived from an amino acid residue of a polypeptide. In some embodiments, a non-hydrocarbon staple comprises two sulfur atom derived from two different amino acid residues of a polypeptide. In some embodiments, a non-hydrocarbon staple comprises two sulfur atoms derived from two different cysteine residues of a polypeptide. In some embodiments, a staple is a cysteine staple. In some embodiments, a staple is a non-cysteine staple.

In some embodiments, the present disclosure provides a stapled peptide agent having a structure:
[X¹]ₚ₁[X²]ₚ₂[X³]ₚ₃[X⁴]ₚ₄[X⁵]ₚ₅[X⁶]ₚ₆[X⁷]ₚ₇[X⁸]ₚ₈[X⁹]ₚ₉[X¹⁰]ₚ₁₀[X¹¹]ₚ₁₁[X¹²]ₚ₁₂[X¹³]ₚ₁₃-X¹⁴X¹⁵X¹⁶X¹⁷X¹⁸X¹⁹-[X²⁰]ₚ₂₀[X²¹]ₚ₂₁[X²²]ₚ₂₂[X²³]ₚ₂₃[X²⁴]ₚ₂₄[X²⁵]ₚ₂₅[X²⁶]ₚ₂₆[X²⁷]ₚ₂₇[X²⁸]ₚ₂₈[X²⁹]ₚ₂₉[X³⁰]ₚ₃₀[X³¹]ₚ₃₁[X³²]ₚ₃₂,
wherein:
   each of p1, p2, p3, p4, p5, p6, p7, p8, p9, p10, p11, p12, p13, p20, p21, p22, p23, p24, p25, p26, p27, p28, p29, p30, p31, p32 is independently 0 or 1;
   each of X¹ to X³² is independently an amino acid residue;
   at least two of X¹ to X³² comprise side chains that are optionally linked together to form a staple.

In some embodiments, a provided peptide is a stapled peptide, and at least two of X¹ to X³² comprise side chains that are linked together to form a staple. In some embodiments, a provided peptide is an unstapled peptide, wherein at least two of X¹ to X³² comprise side chains that can be linked together to form a staple. In some embodiments, a stapled peptide, or an unstapled peptide once stapled, interacts with a target of interest at one or more sites on the target of interest.

In some embodiments, each of X¹ to X³² is independently an amino acid residue of an amino acid having the structure of formula A-I.

In some embodiments, Xⁱ and X^{i+m}, each independently comprises a side chain comprising a thiol, and the two side chains can be linked together to form a staple. In some embodiments, Xⁱ and X^{i+m}, each independently comprises a cysteine side chain, and the two side chains can be linked together to form a cysteine staple.

In some embodiments, Xⁱ and X^{i+m}, each independently comprises a side chain that comprises an olefin. In some embodiments, both of the olefins are terminal olefins. In some embodiments, at least one of Xⁱ and X^{i+m} comprises a side chain comprising an olefin and a nitrogen atom. In some embodiments, at least one of Xⁱ and X^{i+m} comprises —C(R^{2a})(R^{3a}) being -C(-L^{a}-R′)(R^{3a}), wherein at least one methylene unit of L^{a} is replaced with —N(R′)— and R′ comprises an olefin. In some embodiments, at least one of Xⁱ and X^{i+m} comprises —C(R^{2a})(R^{3a}) being —C(-L^{a}-CH═CH₂)(R^{3a}), wherein at least one methylene unit of L^{a} is replaced with —N(R′)—.

In some embodiments, i is an integer of 1 to 31 inclusive, and is m an integer of 1 to 12 inclusive.

In some embodiments, i is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31. In some embodiments, i is 1. In some embodiments, i is 2. In some embodiments, i is 3. In some embodiments, i is 4. In some embodiments, i is 5. In some embodiments, i is 6. In some embodiments, i is 7. In some embodiments, i is 8. In some embodiments, i is 9. In some embodiments, i is 10. In some embodiments, i is 11. In some embodiments, i is 12. In some embodiments, i is 13. In some embodiments, i is 14. In some embodiments, i is 15. In some embodiments, i is 16. In some embodiments, i is 17. In some embodiments, i is 18. In some embodiments, i is 17. In some embodiments, i is 18. In some embodiments, i is 19. In some embodiments, i is 20. In some embodiments, i is 21. In some embodiments, i is 22. In some embodiments, i is 23. In some embodiments, i is 24. In some embodiments, i is 25. In some embodiments, i is 26. In some embodiments, i is 27. In some embodiments, i is 28. In some embodiments, i is 29. In some embodiments, i is 30. In some embodiments, i is 31. In some embodiments, i is 32.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12.

In some embodiments, a stapled peptide comprise one or more staples. In some embodiments, a stapled peptide comprises one and no more than one staple. In some embodiments, a stapled peptide comprises one and no more than one staples from cysteine stapling. In some embodiments, a stapled peptide comprises one and no more than one staples having the structure of

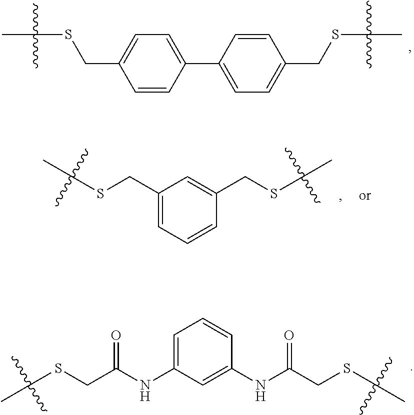 [SMILES: *SCc1ccc(-c2ccc(CS*)cc2)cc1 ; *SCc1cccc(CS*)c1 ; *SCC(=O)Nc1cccc(NC(=O)CS*)c1]

In some embodiments, a stapled peptide comprises one and no more than one staples having the structure of

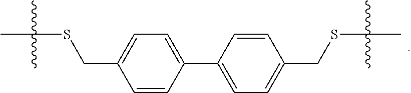 [SMILES: *SCc1ccc(-c2ccc(CS*)cc2)cc1]

In some embodiments, a stapled peptide comprises one and no more than one staples having the structure of

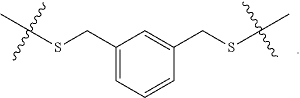 [SMILES: *SCc1cccc(CS*)c1]

In some embodiments, a stapled peptide comprises one and no more than one staples having the structure of

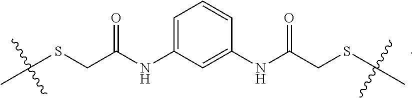 [SMILES: *SCC(=O)Nc1cccc(NC(=O)CS*)c1]

In some embodiments, a stapled peptide comprises no staples having the structure of

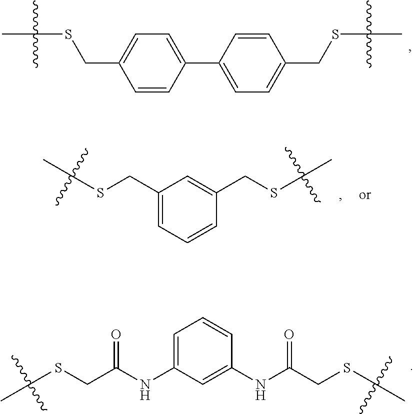 [SMILES: *SCc1ccc(-c2ccc(CS*)cc2)cc1 ; *SCc1cccc(CS*)c1 ; *SCC(=O)Nc1cccc(NC(=O)CS*)c1]

In some embodiments, a stapled peptide comprises no staples having the structure of

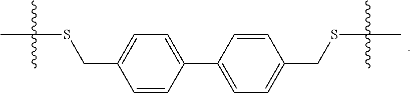 [SMILES: *SCc1ccc(-c2ccc(CS*)cc2)cc1]

In some embodiments, a stapled peptide comprises no staples having the structure of

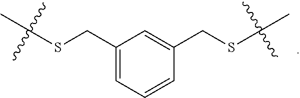 [SMILES: *SCc1cccc(CS*)c1]

In some embodiments, a stapled peptide comprises no staples having the structure of

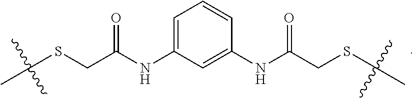 [SMILES: *SCC(=O)Nc1cccc(NC(=O)CS*)c1]

In some embodiments, peptides, e.g., staple peptides, of the present disclosure is or comprises a helix structure. As those skilled in the art will appreciate, helixes can have various lengths. In some embodiments, lengths of helixes range from 5 to 30 amino acid residues. In some embodiments, a length of a helix is 5, 6, 7, 8, 9, 10, 11, 12, 13, 14, 15, 16, 17, 18, 19, or 20, or more, amino acid residues. In some embodiments, a length of a helix is 6 amino acid residues. In some embodiments, a length of a helix is 8 amino acid residues. In some embodiments, a length of a helix is 10 amino acid residues. In some embodiments, a length of a helix is 12 amino acid residues. In some embodiments, a length of a helix is 14 amino acid residues. In some embodiments, a length of a helix is 16 amino acid residues. In some embodiments, a length of a helix is 17 amino acid residues. In some embodiments, a length of a helix is 18 amino acid residues. In some embodiments, a length of a helix is 19 amino acid residues. In some embodiments, a length of a helix is 20 amino acid residues.

In some embodiments, stapled peptides form or comprise alpha-helix structures. In some embodiments, stapling promotes and/or enhances formation of alpha-helix structures. In some embodiments, hydrophilic collapse cloaks amide protons. In some embodiments, helix formation expels high-energy water molecules. In some embodiments, helix formation retains weakly-bound water molecules. In some embodiments, stapled peptides cross membranes at higher efficiencies relative to comparable non-stapled peptides. In some embodiments, staples constrain peptide backbones of stapled peptides or portions thereof to adopt alpha-helical structures. In some embodiments, alpha-helical structures present side chains that confer target-specific recognition and/or drug-like properties. In some embodiments, technologies of present disclosure provide high levels of cytoplasmic and/or nuclear exposure, in some embodiments, by passive membrane permeability. In some embodiments, for increased permeability a hydrophobic amino acid residue may be utilized over a polar residue or a charged residue. In some embodiments, for increased permeability a polar amino acid residue may be utilized over a charged residue. In some embodiments, it is observed that replacing carbon-carbon double bond with a carbon-carbon single bond does not significantly impact cell permeability. In some embodiments, it is observed that staple type and/or positioning impacts cytosolic exposure. In some embodiments, longer staples provide higher cytosolic exposure. In some embodiments, less polar staples may provide higher cytosolic exposure. In some embodiments, stapled peptides provide suppressed renal clearance and long-circulating half-lives. In some embodiments, stapled peptides have high proteolytic stability, e.g., in cells and in vivo relative to comparable non-stapled peptides. In some embodiments, stapled peptides are non- or low-immunogenic. In some embodiments, stapled peptides do not bind MHC class I and/or class II receptor. In some embodiments, stapled peptides provide oral bioavailability, in some cases including and especially in left-handed (D-configurated) form.

### Cysteine Stapling

In some embodiments, the present disclosure provides useful technologies relating to cysteine stapling. Among other things, the present disclosure appreciates that peptides amenable to cysteine stapling and/or comprising one or more cysteine staples, can be produced and/or assessed in a biological system. The present disclosure further appreciates that certain such systems permit development, production, and/or assessment of cysteine stapled peptides having a range of different structures (e.g., different amino acid sequences), and in fact can provide a user with complete control over selection and implementation of amino acid sequences to be incorporated into stapled peptides.

Cysteine stapling, as described herein, involves linking one cysteine residue to another cysteine residue, where the resulting bond is not through the peptide backbone between the linked cysteine residues.

In some embodiments, a stapled peptide as described herein comprises a staple which staple is L^{s}, wherein:
L^{s} is -L^{s1}-S-L^{s2}-S-L^{s3}-;
L^{s1}, L^{s2} and L^{s3} are each independently L;
each L is independently a covalent bond, or an optionally substituted, bivalent C₁-C₂₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L^{s2} is L and comprises at least one —C(O)-.

As described herein, various linker moieties or staples can be L. In some embodiments, L is a covalent bond. In some embodiments, L is an optionally substituted, bivalent C₁-C₂₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O-. In some embodiments, L is an optionally substituted, bivalent C₁-C₂₀ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂-, —S(O)₂N(R′)—, —C(O)S—, or —C(O)O-. In some embodiments, L is an optionally substituted, bivalent C₁-C₁₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O-. In some embodiments, L is an optionally substituted, bivalent C₁-C₁₀ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L is an optionally substituted, bivalent C₁-C₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L is independently a bivalent C₁-C₂₅ aliphatic group. In some embodiments, L is independently a bivalent C₁-C₂₀ aliphatic group. In some embodiments, L is independently a bivalent C₁-C₁₀ aliphatic group. In some embodiments, L is independently a bivalent C₁-C₅ aliphatic group. In some embodiments, L is independently a bivalent C₁ aliphatic group. In some embodiments, L is optionally substituted —CH₂—. In some embodiments, L is —CH₂—.

In some embodiments, L^{s1} is L as described herein. In some embodiments, L^{s1} is optionally substituted —CH₂—. In some embodiments, L^{s1} is —CH₂—. In some embodiments, L^{s3} is L as described herein. In some embodiments, L^{s3} is optionally substituted —CH₂—. In some embodiments, L^{s3} is —CH₂—. In some embodiments, L^{s1} and L^{s3} are both optionally substituted —CH₂—. In some embodiments, L^{s1} and L^{s3} are both —CH₂—. In some embodiments, L^{s} is —CH₂—S-L^{s2}-S—CH₂— wherein L^{s2} is as described herein.

In some embodiments, L^{s2} is L as described herein. In some embodiments, L^{s2} comprises —C(R′)₂-L′-C(R′)₂—, wherein L′ is described in the present disclosure. In some embodiments, L^{s2} is -L^{x1}-C(O)Q-L′-QC(O)-L^{x1}-, wherein each variable is independently as described in the present disclosure. In some embodiments, L^{s2} is —CH₂C(O)Q-L′-QC(O)CH₂—, wherein each —CH₂— is independently and optionally substituted. In some embodiments, L^{s2} is —CH₂C(O)Q-L′-QC(O)CH₂—.

In some embodiments, L^{s2} In some embodiments, L^{s2} is L and comprises at least one —C(O)-. In some embodiments, L^{s2} is L and comprises at least two -C(O)-. In some embodiments, L^{s2} is L and comprises at least one —C(O)Q-, wherein Q is selected from the group consisting of: a covalent bond, —N(R′)—, —O—, and —S—. In some embodiments, L^{s2} is L and comprises at least one —C(O)Q-, wherein Q is selected between —N(R′)— and —O—. In some embodiments, L^{s2} is L and comprises at least two —C(O)Q-, wherein Q is selected from the group consisting of: —N(R′)—, —O—, and —S—. In some embodiments, L^{s2} is L and comprises at least two —C(O)Q-, wherein Q is selected between —N(R′)— and —O—. In some embodiments, L^{s2} is L and comprises at least one —C(O)N(R′)—. In some embodiments, L^{s2} is L and comprises at least two —C(O)N(R′)—. In some embodiments, L^{s2} is L and comprises at least one —C(O)O—. In some embodiments, L^{s2} is L and comprises at least two —C(O)O—.

In some embodiments, L^{s2} comprises -Q-L′-Q-, wherein Q is independently selected from the group consisting of: —N(R′)—, —O—, and —S, wherein L′ is described in the present disclosure.

In some embodiments, L^{s2} comprises -Q-L′-Q-, wherein Q is independently selected between —N(R′)— and —O—, wherein L′ is described in the present disclosure. In some embodiments, L^{s2} comprises —C(O)Q-L′-QC(O)—, wherein Q is independently selected from the group consisting of: —N(R′)—, —O—, and —S, wherein L′ is described in the present disclosure. In some embodiments, L^{s2} comprises —C(O)Q-L′-QC(O)—, wherein Q is independently selected between —N(R′)— and -0, wherein L′ is described in the present disclosure. In some embodiments, L^{s2} comprises —C(R′)₂C(O)Q-L′-QC(O)C(R′)₂—, wherein Q is independently selected from the group consisting of: —N(R′)—, —O—, and —S, wherein L′ is described in the present disclosure. In some embodiments, L^{s2}comprises —C(R′)₂C(O)Q-L′-QC(O)C(R′)₂—, wherein Q is independently selected between —N(R′)— and -0, wherein L′ is described in the present disclosure.

In some embodiments, L^{s2} comprises —N(R′)-L′-N(R′)—, wherein L′ is described in the present disclosure. In some embodiments, L^{s2} comprises —C(O)N(R′)-L′-N(R′)C(O)-, wherein L′ is described in the present disclosure. In some embodiments, L^{s2} is —C(R′)₂C(O)N(R′)-L′-N(R′)C(O)C(R′)₂—, wherein L′ is described in the present disclosure.

In some embodiments, L^{s2} comprises —O(R′)-L′-O(R′)—, wherein L′ is described in the present disclosure. In some embodiments, L^{s2} comprises —C(O)O-L′-OC(O)-, wherein L′ is described in the present disclosure. In some embodiments, L^{s2} is —C(R′)₂C(O)O-L′-OC(O)C(R′)₂—, wherein L′ is described in the present disclosure.

In some embodiments, R′ is an optionally substituted C₁₋₃₀ aliphatic. In some embodiments, R′ is an optionally substituted C₁₋₁₅ aliphatic. In some embodiments, R′ is an optionally substituted C₁₋₁₀ aliphatic. In some embodiments, R′ is an optionally substituted C₁₋₅ aliphatic. In some embodiments, R′ is hydrogen.

In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₈ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₃ aliphatic. In some embodiments, L′ is optionally substituted bivalent C₁-C₂ aliphatic. In some embodiments, L′ is optionally substituted bivalent C₁ aliphatic. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—.

In some embodiments, L′ is optionally substituted bivalent C₆₋₂₀ aryl ring. In some embodiments, L′ is optionally substituted bivalent C₆₋₁₄ aryl ring. In some embodiments, L′ is optionally substituted bivalent C₆₋₁₀ aryl ring. In some embodiments, L′ is optionally substituted bivalent C₆ aryl ring. In some embodiments, L′ is bivalent C₆ aryl substituted with at least one halogen. In some embodiments, L′ is bivalent C₆ aryl substituted with at least two halogen. In some embodiments, L′ is bivalent C₆ aryl substituted with at least three halogen. In some embodiments, L′ is bivalent C₆ aryl substituted with four halogen. In some embodiments, L′ is bivalent C₆ aryl substituted with at least one fluorine. In some embodiments, L′ is bivalent C₆ aryl substituted with at least two fluorine. In some embodiments, L′ is bivalent C₆ aryl substituted with at least three fluorine. In some embodiments, L′ is bivalent C₆ aryl substituted with four fluorine. In some embodiments, L′ is bivalent C₆ aryl substituted with at least one chlorine. In some embodiments, L′ is bivalent C₆ aryl substituted with at least two chlorine. In some embodiments, L′ is bivalent C₆ aryl substituted with at least three chlorine. In some embodiments, L′ is bivalent C₆ aryl substituted with four chlorine. In some embodiments, L′ is bivalent C₆ aryl substituted at with least one —O(CH₂)₀₋₄CH₃. In some embodiments, L′ is bivalent C₆ aryl substituted with at least two —O(CH₂)₀₋₄CH₃. In some embodiments, L′ is bivalent C₆ aryl substituted with at least three —O(CH₂)₀₋₄CH₃. In some embodiments, L′ is bivalent C₆ aryl substituted with four —O(CH₂)₀₋₄CH₃.

In some embodiments, L′ is bivalent 5-20 membered heteroaryl ring having 1-10 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, and sulfur. In some embodiments, L′ is bivalent 6 membered heteroaryl ring having 1-2 heteroatoms independently selected from oxygen, nitrogen, and sulfur. In some embodiments, L′ is bivalent 6 membered heteroaryl ring having 2 nitrogen.

In some embodiments, L′ is optionally substituted bivalent C₃₂₀ cycloaliphatic ring. In some embodiments, L′ is optionally substituted bivalent C₃₋₁₅ cycloaliphatic ring. In some embodiments, L′ is optionally substituted bivalent C₃₋₁₀ cycloaliphatic ring. In some embodiments, L′ is optionally substituted bivalent C₃₋₉ cycloaliphatic ring. In some embodiments, L′ is optionally substituted bivalent C₃₋₈ cycloaliphatic ring. In some embodiments, L′ is optionally substituted bivalent C₃₋₇ cycloaliphatic ring. In some embodiments, L′ is optionally substituted bivalent C₃₋₆ cycloaliphatic ring. In some embodiments, L′ is optionally substituted bivalent C₃₋₅ cycloaliphatic ring. In some embodiments, L′ is optionally substituted bivalent C₃₋₄ cycloaliphatic ring. In some embodiments, L′ is optionally substituted bivalent C₃ cycloaliphatic ring. In some embodiments, L′ is optionally substituted bivalent C₄ cycloaliphatic ring. In some embodiments, L′ is optionally substituted bivalent C₅ cycloaliphatic ring. In some embodiments, L′ is optionally substituted bivalent C₅ cycloalkyl ring. In some embodiments, L′ is optionally substituted bivalent C₅ cycloalkenyl ring. In some embodiments, L′ is optionally substituted bivalent C₆ cycloaliphatic ring. In some embodiments, L′ is optionally substituted bivalent C₆ cycloalkyl ring.

In some embodiments, L^{s2} comprises —N(R′)-L′—N(R′)— and L′ is a covalent bond. In some embodiments L^{s2} comprises —N(R)—N(R)—, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments L^{s2} comprises —N(R)—N(R)—, wherein:
each R is independently optionally substituted C₁₋₃₀ aliphatic; or
two or more R groups on two or more atoms are optionally and independently taken together with their intervening atoms to form an optionally substituted, 3-30 membered monocyclic ring.

In some embodiments, L^{s2} is a staple selected from the group consisting of:

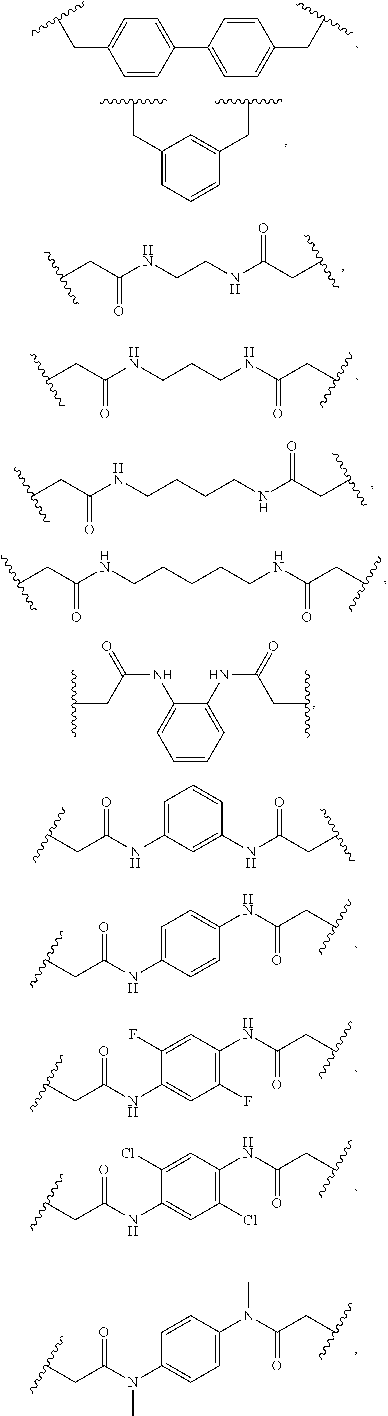

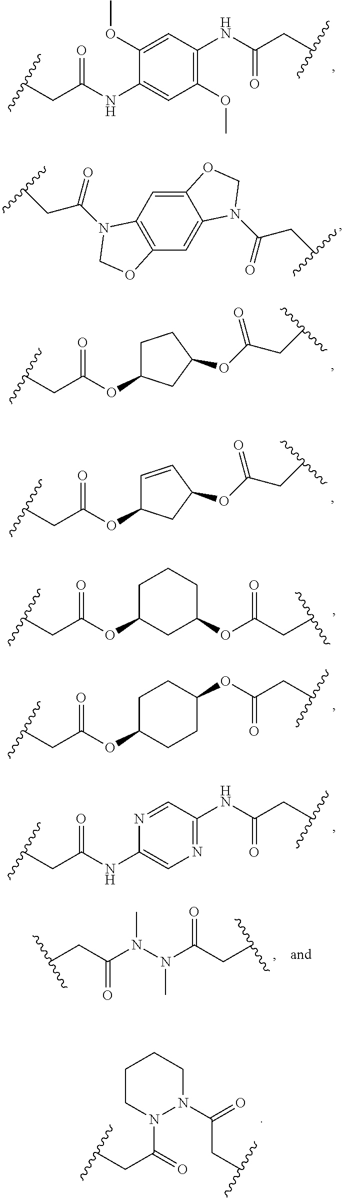

As those skilled in the art will appreciate, provided technologies can be utilized to prepare collection of peptides using non-cysteine residues and suitable chemistry therefor. For example, in some embodiments, cysteine stapling is replaced with lysine stapling, wherein the cysteine residues for cysteine stapling are replaced with lysine residues for lysine stapling (e.g., using agents that can crosslink two lysine residues, for example, through reactions with side chain amino groups). In some embodiments, for lysine stapling, R^{E} in various formulae is or comprises an activated carboxylic acid group (e.g., NHS ester group), an imidoester group, etc. Suitable reagents are widely known in the art including many commercially available ones. In some embodiments, cysteine stapling is replaced with methionine stapling. In some embodiments, cysteine residues for cysteine stapling are replaced with methionine residues for methionine stapling. In some embodiments, cysteine stapling is replaced with tryptophan stapling. In some embodiments, cysteine residues for cysteine stapling are replaced with tryptophan residues for tryptophan stapling. As those skilled in the art will appreciate, various technologies (e.g., reagents, reactions, etc.) are described in the art and can be utilized in accordance with the present disclosure for, e.g., methionine stapling, tryptophan stapling, etc. In some embodiments, such stapling can be performed using reagents having various formulae described herein, wherein R^{E} is or comprises a group that are suitable for methionine and/or tryptophan stapling. In some embodiments, stapling may be performed using one residue at a first position, and a different residue at a second position. Useful reagents for such stapling may comprise a first reactive group for stapling at a first position (e.g., through a first R^{E}), and a second reactive group for stapling at a second position (e.g., through a second R^{E}).

In some embodiments, for various types of stapling (e.g., cysteine stapling, or non-cysteine stapling), stapling is between residues (e.g., cysteine residues for cysteine stapling) separated by two residues (i+3 stapling). In some embodiments, stapling is between residues separated by three residues (i+4 stapling). In some embodiments, stapling is between residues separated by six residues (i+7 stapling).

As appreciated by those skilled in the art, in some embodiments, more than two residues can be stapled at the same time. For example, in some embodiments, three or more cysteines are stapled using crosslinking reagents containing three or more reactive groups (e.g., R^{E} groups).

### Non-Cysteine Stapling

In some embodiments, the present disclosure provides useful technologies relating to non-cysteine stapling. Among other things, the present disclosure appreciates that peptides amenable to cysteine stapling and/or comprising one or more non-cysteine staples, can have its cysteine residues and cysteine staple replaced with other amino acids and staples (e.g. hydrocarbon and other non-hydrocarbon amino acid and staples). In some embodiments, the resulting non-cysteine stapled peptide maintains the same or similar interaction with a target of interest when compared to a reference cysteine stapled peptide. Described herein are non-cysteine amino acids and non-cysteine staples involving such non-cysteine amino acid residues.

In some embodiments, an amino acid of formula A-I is a compound having the structure of formula A-II:

NH(R^{a1})-L^{a1}-C(-L^{a}-CH═CH₂)(R^{a3})-L^{a2}-COOH, A-II

or a salt thereof, wherein each variable is independently as described in the present disclosure.

In some embodiments, an amino acid of formula A-I is a compound having the structure of formula A-III:

NH(R^{a1})-C(-L^{a}-CH═CH₂)(R^{a3})—COOH, A-III

or a salt thereof, wherein each variable is independently as described in the present disclosure.

In some embodiments, L^{a} comprises at least one —N(R′)— wherein R′ is independently as described in the present disclosure.

In some embodiments, an amino acid of formula A-I is a standard amino acid. In some embodiments, an amino acid of formula A-I is selected from Tables A-I, A-II, and A-III:

TABLE A-I
Exemplary amino acids (Fmoc-Protected).
Monomer A (M_{A})

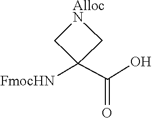

Monomer B (M_{B})

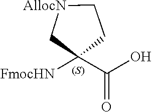

Monomer C (M_{C})

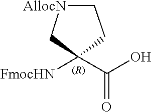

TABLE A-II
Exemplary amino acids (Fmoc-Protected).
Monomer D (M_{D})

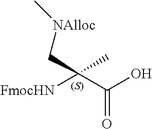

Monomer E (M_{E})

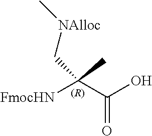

Monomer F (M_{F})

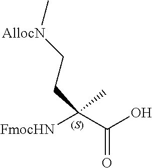

Monomer G (M_{G})

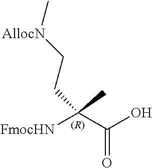

Monomer H (M_{H})

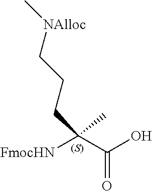

Monomer I (M_{I})

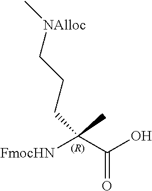

TABLE A-III
Exemplary amino acids (Fmoc-Protected).
S₃

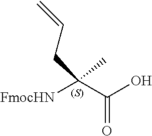

R₃

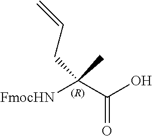

S₄

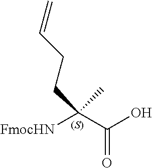

R₄

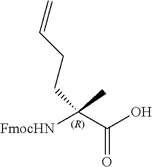

S₅

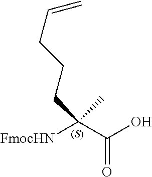

R₅

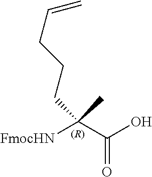

B₅

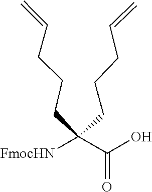

S₆

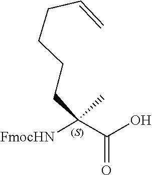

R₆

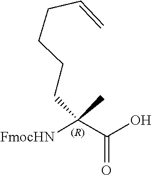

S₇

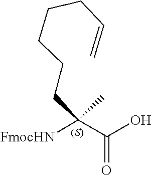

R₇

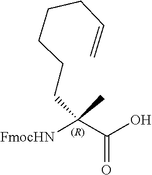

S₈

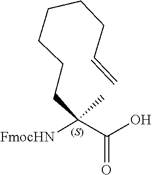

R₈

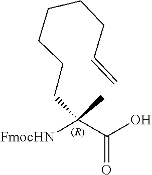

In some embodiments, an amino acid is an alpha-amino acid. In some embodiments, an amino acid is an L-amino acid. In some embodiments, an amino acid is a D-amino acid. In some embodiments, the alpha-carbon of an amino acid is achiral. In some embodiments, an amino acid is a beta-amino acid. In some embodiments, an amino acid is a gamma-amino acid.

In some embodiments, a provided amino acid sequence contains two or more amino acid residues whose side chains are linked together to form one or more staples. In some embodiments, a provided amino acid sequence contains two or more amino acid residues, each of which independently has a side chain comprising an olefin. In some embodiments, a provided amino acid sequence contains two or more amino acid residues, each of which independently has a side chain comprising a terminal olefin. In some embodiments, a provided amino acid sequence contains two and no more than two amino acid residues, each of which independently has a side chain comprising an olefin. In some embodiments, a provided amino acid sequence contains two and no more than two amino acid residues, each of which independently has a side chain comprising a terminal olefin. In some embodiments, a provided amino acid sequence comprises at least one residue of an amino acid that comprises an olefin and a nitrogen atom other than the nitrogen atom of its amino group. In some embodiments, a provided amino acid sequence comprises at least one residue of an amino acid that comprises a terminal olefin and a nitrogen atom other than the nitrogen atom of its amino group. In some embodiments, a provided amino acid sequence comprises at least one residue of an amino acid that has a side chain than comprises a terminal olefin and a nitrogen atom. In some embodiments, a provided amino acid sequence comprises at least one residue of an amino acid of formula A-I, wherein R^{a2} comprising an olefin and a —N(R′)— moiety, wherein R′ is as described in the present disclosure (including, in some embodiments, optionally taken together with R^{a3} and their intervening atoms to form an optionally substituted ring as described in the present disclosure). In some embodiments, R^{a2} comprising a terminal olefin and a —N(R′)— moiety wherein R′ is as described in the present disclosure. In some embodiments, a provided amino acid sequence comprises at least one residue of an amino acid selected from Table A-I. In some embodiments, a provided amino acid sequence comprises at least one residue of an amino acid selected from Table A-II. In some embodiments, a provided amino acid sequence comprises at least one residue of an amino acid selected from Table A-III. In some embodiments, two olefins from two side chains are linked together through olefin metathesis to form a staple. In some embodiments, a staple is preferably formed by side chains of amino acid residues that are not at the corresponding positions of a target of interest. In some embodiments, a formed staple does not disrupt interaction between the peptide and a target of interest.

In some embodiments, a provided staple is a hydrocarbon staple. In some embodiments, a hydrocarbon staple comprises no chain heteroatoms wherein a chain of a staple is the shortest covalent connection within the staple from one end of the staple to the other end of the staple.

In some embodiments, a provided staple is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is a carbamate staple in that it comprises a —N(R′)-C(O)-O— moiety in its chain. In some embodiments, a non-hydrocarbon staple is an amino staple in that it comprises a —N(R′)— moiety in its chain, wherein the —N(R′)— moiety is not part of —N(R′)-C(O)-O-. In some embodiments, a non-hydrocarbon staple is an amino staple in that it comprises a —N(R′)— moiety in its chain, wherein the —N(R′)— moiety is not bonded to a carbon atom that additionally forms a double bond with a heteroatom (e.g., —C(═O), —C(═S), —C(═N-R′), etc.).

In some embodiments, a provided stapled peptide comprises a staple which staple is L^{s}, wherein L^{s} is -L^{s1}-L^{s2}-L^{s3}-, each of L^{s1}, L^{s2}, and L^{s3} is independently L, wherein each L is independently as described in the present disclosure. In some embodiments, a provided staple is L^{s}.

In some embodiments, L^{s1} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s1} is -L′-N(R′)—, wherein L′ is optionally substituted bivalent C₁-C₁₉ aliphatic. In some embodiments, L^{s1} is -L′-N(CH₃)—, wherein L′ is optionally substituted bivalent C₁-C₁₉ aliphatic.

In some embodiments, R′ is optionally substituted C₁₋₆ alkyl. In some embodiments, R′ is C₁₋₆ alkyl. In some embodiments, R′ is methyl. In some embodiments, the peptide backbone atom to which L^{s1} is bonded is also bonded to R¹, and R′ and R¹ are both R and are taken together with their intervene atoms to form an optionally substituted ring as described in the present disclosure. In some embodiments, a formed ring has no additional ring heteroatoms in addition to the nitrogen atom to which R′ is bonded. In some embodiments, a formed ring is 3-membered. In some embodiments, a formed ring is 4-membered. In some embodiments, a formed ring is 5-membered. In some embodiments, a formed ring is 6-membered.

In some embodiments, L′ is optionally substituted bivalent C₁-C₂₀ aliphatic. In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₈ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₄ aliphatic. In some embodiments, L′ is optionally substituted alkylene. In some embodiments, L′ is optionally substituted alkenylene. In some embodiments, L′ is unsubstituted alkylene. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—. In some embodiments, L′ is bonded to a peptide backbone atom. In some embodiments, L′ is optionally substituted alkenylene. In some embodiments, L′ is unsubstituted alkenylene. In some embodiments, L′ is —CH₂—CH═CH-CH₂—.

In some embodiments, L^{s1} comprises at least one —N(R′)C(O)—, wherein R′ is as described in the present disclosure. In some embodiments, L^{s1} is -L′-N(R′)C(O)—, wherein each of L′ and R′ is independently as described in the present disclosure. In some embodiments, L^{s1} is -L′-N(CH₃)C(O)—, wherein L′ is independently as described in the present disclosure.

In some embodiments, L^{s1} is a covalent bond.

In some embodiments, L^{s1} is L′, wherein L′ is as described in the present disclosure.

In some embodiments, L^{s2} is L, wherein L is as described in the present disclosure. In some embodiments, L^{s2} is L′, wherein L′ is as described in the present disclosure. In some embodiments, L^{s2}comprises —CH₂—CH═CH-CH₂—. In some embodiments, L^{s2} is —CH₂—CH═CH-CH₂—. In some embodiments, L^{s2} comprises —(CH₂)₄—. In some embodiments, L^{s2} is —(CH₂)₄—.

In some embodiments, L^{s3} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s3} is -L′-N(R′)—, wherein L′ is optionally substituted bivalent C₁-C₁₉ aliphatic. In some embodiments, L^{s3} is -L′-N(CH₃)—, wherein L′ is optionally substituted bivalent C₁-C₁₉ aliphatic.

In some embodiments, L^{s3} comprises at least one —N(R′)C(O)—, wherein R′ is as described in the present disclosure. In some embodiments, L^{s3} is -L′-N(R′)C(O)—, wherein each of L′ and R′ is independently as described in the present disclosure. In some embodiments, L^{s3} is -L′-N(CH₃)C(O)—, wherein L′ is independently as described in the present disclosure.

In some embodiments, L^{s3} is L′, wherein L′ is as described in the present disclosure. In some embodiments, L^{s3} is optionally substituted alkylene. In some embodiments, L^{s3} is unsubstituted alkylene.

In some embodiments, L^{s} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s} comprises at least one —N(R′)C(O)—, wherein R′ is as described in the present disclosure.

In some embodiments, L comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L is -L′-N(R′)—, wherein L′ is optionally substituted bivalent C₁-C₁₉ aliphatic. In some embodiments, L is -L′-N(CH₃)—, wherein L′ is optionally substituted bivalent C₁-C₁₉ aliphatic.

In some embodiments, L comprises at least one —N(R′)C(O)—, wherein R′ is as described in the present disclosure. In some embodiments, L is -L′-N(R′)C(O)—, wherein each of L′ and R′ is independently as described in the present disclosure. In some embodiments, L is -L′-N(CH₃)C(O)—, wherein L′ is independently as described in the present disclosure.

In some embodiments, L is L′, wherein L′ is as described in the present disclosure. In some embodiments, L is optionally substituted alkylene. In some embodiments, L is unsubstituted alkylene.

In some embodiments, L is optionally substituted bivalent C₁-C₂₅ aliphatic. In some embodiments, L is optionally substituted bivalent C₁-C₂₀ aliphatic. In some embodiments, L is optionally substituted bivalent C₁-C₁₅ aliphatic. In some embodiments, L is optionally substituted bivalent C₁-C₁₀ aliphatic. In some embodiments, Lis optionally substituted bivalent C₁-C₉ aliphatic. In some embodiments, L is optionally substituted bivalent C₁-C₈ aliphatic. In some embodiments, L is optionally substituted bivalent C₁-C₇ aliphatic. In some embodiments, L is optionally substituted bivalent C₁-C₆ aliphatic. In some embodiments, L is optionally substituted bivalent C₁-C₅ aliphatic. In some embodiments, L is optionally substituted bivalent C₁-C₄ aliphatic. In some embodiments, L is optionally substituted alkylene. In some embodiments, L is optionally substituted alkenylene. In some embodiments, L is unsubstituted alkylene. In some embodiments, L is —CH₂—. In some embodiments, L is —(CH₂)₂—. In some embodiments, L is —(CH₂)₃—. In some embodiments, L is —(CH₂)₄—. In some embodiments, L is —(CH₂)₅—. In some embodiments, L is —(CH₂)₆—. In some embodiments, L is —(CH₂)₇—. In some embodiments, L is —(CH₂)₈—. In some embodiments, L is bonded to a peptide backbone atom. In some embodiments, L is optionally substituted alkenylene. In some embodiments, L is unsubstituted alkenylene. In some embodiments, L is —CH₂—CH═CH-CH₂—.

In some embodiments, each R is independently —H, or an optionally substituted group selected from C₁₋₃₀ (e.g., C₁₋₂₀, C₁₋₁₅, C₁₋₁₀, etc.) aliphatic, C₁₋₃₀ (e.g., C₁₋₂₀, C₁₋₁₅, C₁₋₁₀, etc.) heteroaliphatic having 1-10 (e.g., 1-8, 1-6, 1-5, 1-4, 1, 2, 3, 4, 5, 6, 7, 8, 9, or 10, etc.) heteroatoms independently selected from oxygen, nitrogen, sulfur, phosphorus and silicon, C₆₋₃₀ (e.g., C₆₋₂₀, C₆₋₁₄, C₆₋₁₀, etc.) aryl, C₆₋₃₀ (e.g., C₆₋₂₀, C₆₋₁₄, C₆₋₁₀, etc.) arylaliphatic, C₆₋₃₀ (e.g., C₆₋₂₀, C₆₋₁₄, C₆₋₁₀, etc.) arylheteroaliphatic having 1-10 (e.g., 1-8, 1-6, 1-5, 1-4, 1, 2, 3, 4, 5, 6, 7, 8, 9, or 10, etc.) heteroatoms independently selected from oxygen, nitrogen, sulfur, phosphorus and silicon, 5-30 (e.g., 5-20, 5-14, 5-10, 5-6, 5, 6, 9, 10, etc.) membered heteroaryl having 1-10 (e.g., 1-8, 1-6, 1-5, 1-4, 1, 2, 3, 4, 5, 6, 7, 8, 9, or 10, etc.) heteroatoms independently selected from oxygen, nitrogen, sulfur, phosphorus and silicon, and 3-30 (e.g., 3-20, 3-15, 3-10, 3-9, 3-6, 5-10, 3, 4, 5, 6, 7, 8, 9, or 10, etc.) membered heterocyclyl having 1-10 (e.g., 1-8, 1-6, 1-5, 1-4, 1, 2, 3, 4, 5, 6, 7, 8, 9, or 10, etc.) heteroatoms independently selected from oxygen, nitrogen, sulfur, phosphorus and silicon. In some embodiments, two R groups are optionally and independently taken together to form a covalent bond. In some embodiments, two or more R groups on the same atom are optionally and independently taken together with the atom to form an optionally substituted, 3-30 (e.g., 3-20, 3-15, 3-10, 3-9, 3-6, 5-10, 3, 4, 5, 6, 7, 8, 9, or 10, etc.) membered, monocyclic, bicyclic or polycyclic ring having, in addition to the atom, 0-10 (e.g., 1-10, 1-8, 1-6, 1-5, 1-4, 0, 1, 2, 3, 4, 5, 6, 7, 8, 9, or 10, etc.) heteroatoms independently selected from oxygen, nitrogen, sulfur, phosphorus and silicon. In some embodiments, two or more R groups on two or more atoms are optionally and independently taken together with their intervening atoms to form an optionally substituted, 3-30 (e.g., 3-20, 3-15, 3-10, 3-9, 3-6, 5-10, 3, 4, 5, 6, 7, 8, 9, or 10, etc.) membered, monocyclic, bicyclic or polycyclic ring having, in addition to the intervening atoms, 0-10 (e.g., 1-10, 1-8, 1-6, 1-5, 1-4, 0, 1, 2, 3, 4, 5, 6, 7, 8, 9, or 10, etc.) heteroatoms independently selected from oxygen, nitrogen, sulfur, phosphorus and silicon. In some embodiments, each R is independently —H, or an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₄ aryl, C₆₋₁₄ arylaliphatic, C₆₋₁₄ arylheteroaliphatic having 1-5 heteroatoms independently selected from oxygen, nitrogen, sulfur, phosphorus and silicon, 5-14 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon, or two R groups are optionally and independently taken together to form a covalent bond; or two or more R groups on the same atom are optionally and independently taken together with the atom to form an optionally substituted, 3-10 membered, monocyclic, bicyclic or polycyclic ring having, in addition to the atom, 0-5 heteroatoms independently selected from oxygen, nitrogen, sulfur, phosphorus and silicon; or two or more R groups on two or more atoms are optionally and independently taken together with their intervening atoms to form an optionally substituted, 3-10 membered, monocyclic, bicyclic or polycyclic ring having, in addition to the intervening atoms, 0-5 heteroatoms independently selected from oxygen, nitrogen, sulfur, phosphorus and silicon.

In some embodiments, one end of a staple is connected to an atom Aⁿ¹ of the peptide backbone, wherein Aⁿ¹ is optionally substituted with R¹ and is an atom of an amino acid residue at amino acid position n¹ of the peptide from the N-terminus, and the other end is connected to an atom Aⁿ² of the peptide backbone, wherein Aⁿ² is optionally substituted with R² (in some embodiments, R¹ and/or R² is R which can be hydrogen) and is an atom of an amino acid residue at amino acid position n² of the peptide from the N-terminus, wherein each of n¹ and n² is independently an integer, and n²=n¹+m, wherein m is 3-12.

In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, a staple is referred to a (i, i+m) staple.

In some embodiments, Aⁿ¹ is a carbon atom. In some embodiments, Aⁿ¹ is achiral. In some embodiments, Aⁿ¹ is chiral. In some embodiments, Aⁿ¹ is R. In some embodiments, Aⁿ¹ is S.

In some embodiments, Aⁿ² is a carbon atom. In some embodiments, Aⁿ² is achiral. In some embodiments, Aⁿ² is chiral. In some embodiments, Aⁿ² is R. In some embodiments, Aⁿ² is S.

In some embodiments, Aⁿ¹ is achiral and Aⁿ² is achiral. In some embodiments, Aⁿ¹ is achiral and Aⁿ² is R. In some embodiments, Aⁿ¹ is achiral and Aⁿ² is S. In some embodiments, Aⁿ¹ is R and Aⁿ² is achiral. In some embodiments, Aⁿ¹ is R and Aⁿ² is R. In some embodiments, Aⁿ¹ is R and Aⁿ² is S. In some embodiments, Aⁿ¹ is S and Aⁿ² is achiral. In some embodiments, Aⁿ¹ is S and Aⁿ² is R. In some embodiments, Aⁿ¹ is S and Aⁿ² is S.

In some embodiments, provided stereochemistry at staple-backbone connection points and/or combinations thereof, optionally together with one or more structural elements of provided peptide, e.g., staple chemistry (hydrocarbon, non-hydrocarbon), staple length, etc. can provide various benefits, such as improved preparation yield, purity, and/or selectivity, improved properties (e.g., improved solubility, improved stability, lowered toxicity, improved selectivities, etc.), improved activities, etc. In some embodiments, provided stereochemistry and/or stereochemistry combinations are different from those typically used, e.g., those of U.S. Pat. No. 9,617,309, US 2015-0225471, US 2016-0024153, US 2016-0215036, US 2016-0244494, WO 2017/062518, and provided one or more of benefits described in the present disclosure.

In some embodiments, a staple can be of various lengths, in some embodiments, as represent by the number of chain atoms of a staple. In some embodiments, a chain of a staple is the shortest covalent connection in the staple from a first end (connection point with a peptide backbone) of a staple to a second end of the staple, wherein the first end and the second end are connected to two different peptide backbone atoms. In some embodiments, a staple comprises 5-30 chain atoms, e.g., 5, 6, 7, 8, 9, or 10 to 11, 12, 13, 14, 15, 16, 17, 18, 19, 20, 21, 22, 23, 24, or 25 chain atoms. In some embodiments, a staple comprises 5 chain atoms. In some embodiments, a staple comprises 6 chain atoms. In some embodiments, a staple comprises 7 chain atoms. In some embodiments, a staple comprises 8 chain atoms. In some embodiments, a staple comprises 9 chain atoms. In some embodiments, a staple comprises 10 chain atoms. In some embodiments, a staple comprises 11 chain atoms. In some embodiments, a staple comprises 12 chain atoms. In some embodiments, a staple comprises 13 chain atoms. In some embodiments, a staple comprises 14 chain atoms. In some embodiments, a staple comprises 15 chain atoms. In some embodiments, a staple comprises 16 chain atoms. In some embodiments, a staple comprises 17 chain atoms. In some embodiments, a staple comprises 18 chain atoms. In some embodiments, a staple comprises 19 chain atoms. In some embodiments, a staple comprises 20 chain atoms. In some embodiments, a staple has a length of 5 chain atoms. In some embodiments, a staple has a length of 6 chain atoms. In some embodiments, a staple has a length of 7 chain atoms. In some embodiments, a staple has a length of 8 chain atoms. In some embodiments, a staple has a length of 9 chain atoms. In some embodiments, a staple has a length of 10 chain atoms. In some embodiments, a staple has a length of 11 chain atoms. In some embodiments, a staple has a length of 12 chain atoms. In some embodiments, a staple has a length of 13 chain atoms. In some embodiments, a staple has a length of 14 chain atoms. In some embodiments, a staple has a length of 15 chain atoms. In some embodiments, a staple has a length of 16 chain atoms. In some embodiments, a staple has a length of 17 chain atoms. In some embodiments, a staple has a length of 18 chain atoms. In some embodiments, a staple has a length of 19 chain atoms. In some embodiments, a staple has a length of 20 chain atoms. In some embodiments, a staple has a length of 8-15 chain atoms. In some embodiments, a staple has 8-12 chain atoms. In some embodiments, a staple has 9-12 chain atoms. In some embodiments, a staple has 9-10 chain atoms. In some embodiments, a staple has 8-10 chain atoms. In some embodiments, length of a staple can be adjusted according to the distance of the amino acid residues it connects, for example, a longer staple may be needed for a (i, i+7) staple than a (i, i+4) staple. Staple lengths may be otherwise described. For example, in some embodiments, staple lengths may be described as the total number of chain atoms and non-chain ring atoms, where a non-chain ring atom is an atom of the staple which forms a ring with one or more chain atoms but is not a chain atom in that it is not within the shortest covalent connection from a first end of the staple to a second end of the staple. In some embodiments, staples formed using Monomer A (which comprises a azetidine moiety), Monomer B (which comprises a pyrrolidine moiety), and/or Monomer C (which comprises a pyrrolidine moiety) may comprise one or two non-chain ring atoms as illustrated in the exemplary stapled peptides.

In some embodiments, a staple has no heteroatoms in its chain. In some embodiments, a staple comprises at least one heteroatom in its chain. In some embodiments, a staple comprises at least one nitrogen atom in its chain.

In some embodiments, a staple is L^{s}, wherein L^{s} is an optionally substituted, bivalent C₈₋₁₄ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₉₋₁₃ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₀₋₁₅ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₁₋₁₄ aliphatic group wherein one or more methylene units of the aliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is a (i, i+4) staple in that not including the two amino acid residues that are directly connected to the staple, there are three amino acid residues between the two amino acid residues that are directly connected to the staple. In some embodiments, a staple is a (i, i+7) staple in that not including the two amino acid residues that are directly connected to the staple, there are six amino acid residues between the two amino acid residues that are directly connected to the staple.

In some embodiments, for each of L^{s}, L^{s1}, L^{s2}, and L^{s3}, any replacement of methylene units, if any, is replaced with —N(R′)— or —N(R′)—C(O)—.

In some embodiments, an olefin in a staple is a Z-olefin. In some embodiments, an olefin in a staple in an E-olefin. In some embodiments, a provided composition comprises stapled peptides comprising a staple that contains a Z-olefin and stapled peptides comprising a staple that contains an E-olefin. In some embodiments, a provided composition comprises stapled peptides comprising a staple that contains a Z-olefin. In some embodiments, a provided composition comprises stapled peptides comprising a staple that contains an E-olefin. In some embodiments, otherwise identical stapled peptides that differ only in the E/Z configuration of staple olefin demonstrate different properties and/or activities as demonstrated herein. In some embodiments, stapled peptides with E-olefin in a staple may provide certain desirable properties and/or activities given the context. In some embodiments, stapled peptides with Z-olefin in a staple may provide certain desirable properties and/or activities given the context.

In some embodiments, two staples may be bonded to the same atom of the peptide backbone, forming a “stitch” structure.

In some embodiments, a staple is Pro-lock in that one end of the staple is bonded to the alpha-carbon of a proline residue.

In some embodiments, an exemplary staple is a staple as illustrated below in Tables S-1, S-2, S-3, and S-4 (with exemplary peptide backbone illustrated for clarity (can be applied to other peptide backbone), X being amino acid residues). In some embodiments, the olefin is Z. In some embodiments, the olefin is E. In some embodiments, an (i, i+4) staple is selected from Table S-1. In some embodiments, an (i, i+4) staple is selected from Table S-2. In some embodiments, an (i, i+7) staple is selected from Table S-3. In some embodiments, an (i, i+7) staple is selected from Table S-4.

TABLE S-1
Exemplary staples.

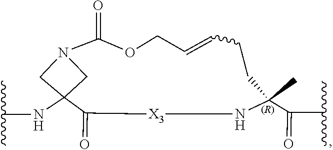

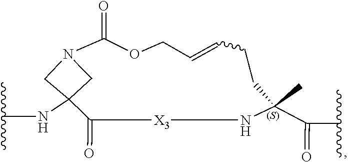

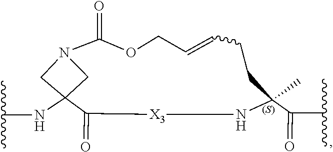

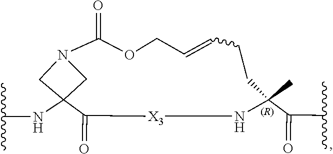

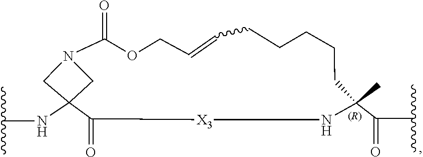

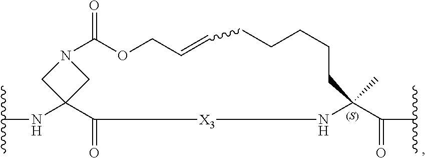

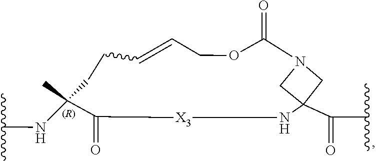

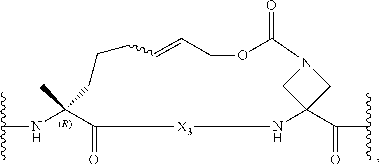

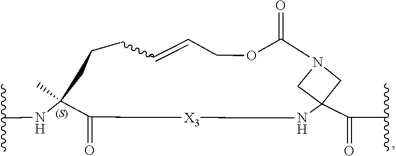

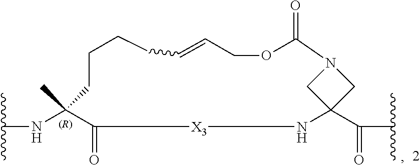

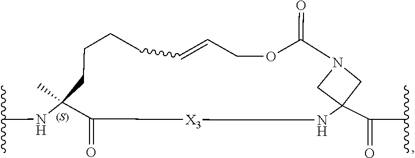

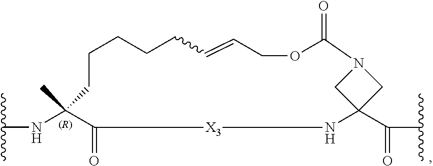

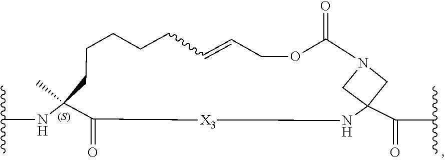

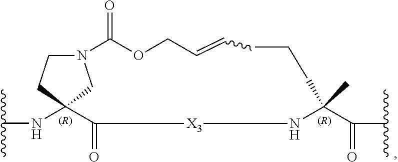

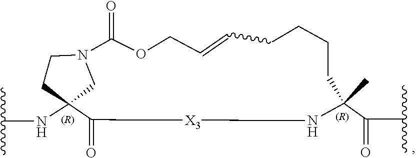

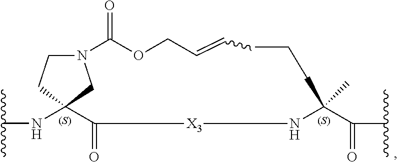

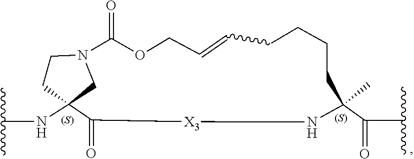

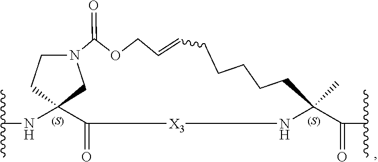

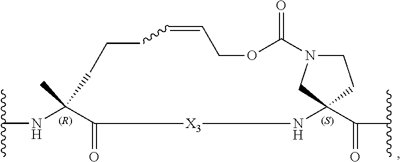

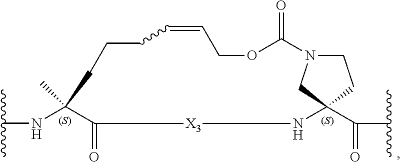

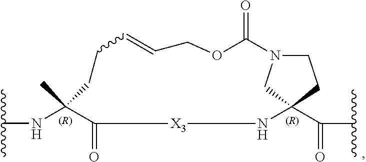

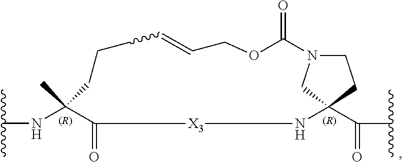

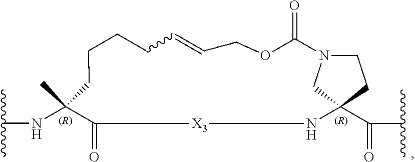

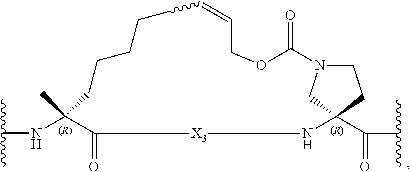

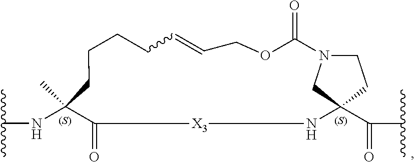

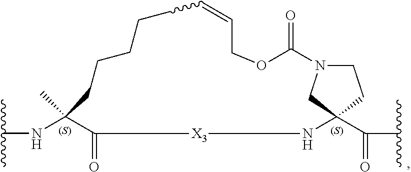

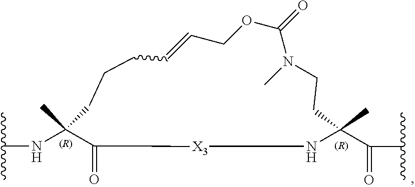

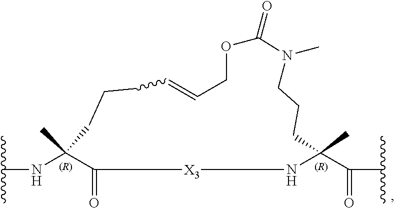

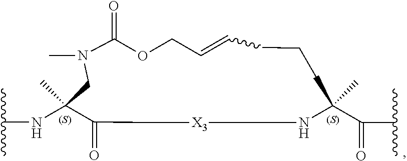

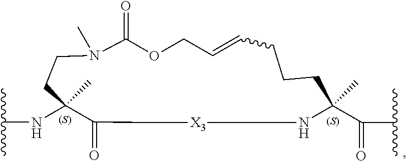

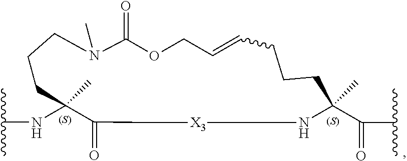

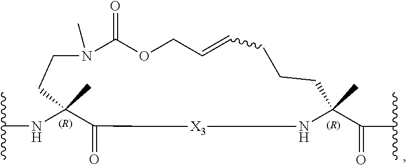

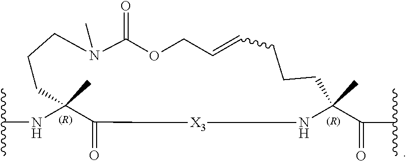

TABLE S-2
Exemplary staples.

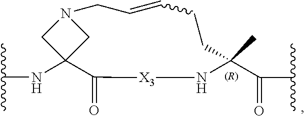

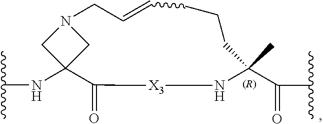

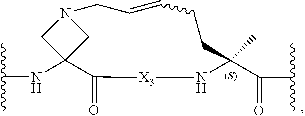

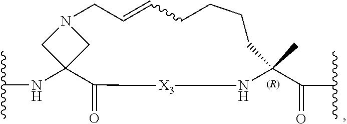

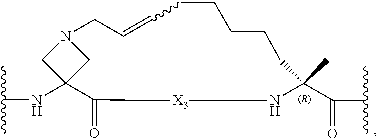

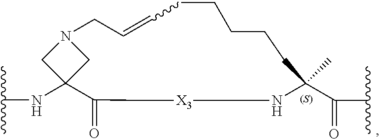

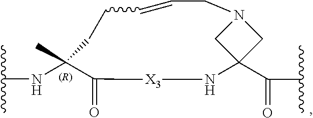

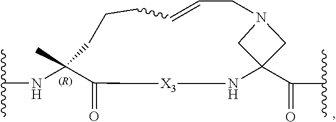

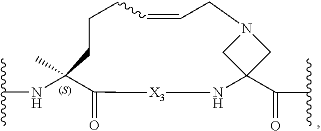

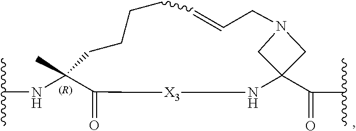

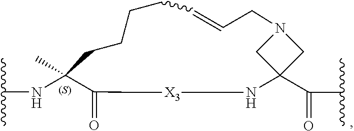

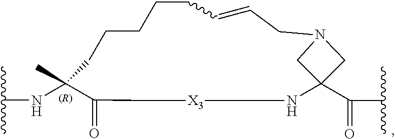

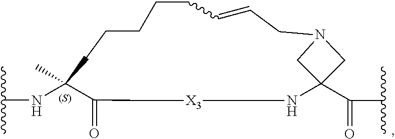

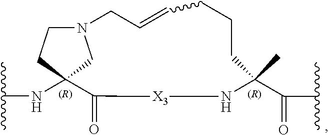

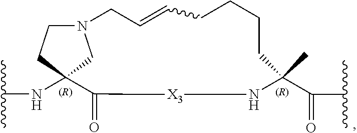

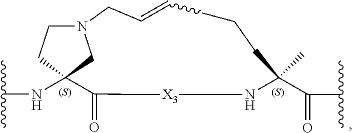

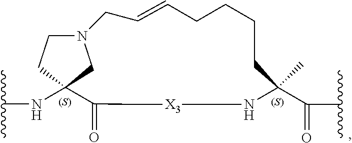

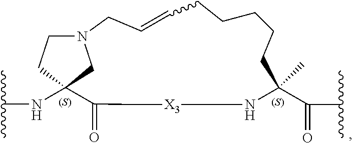

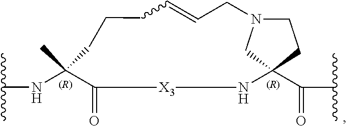

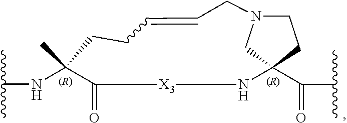

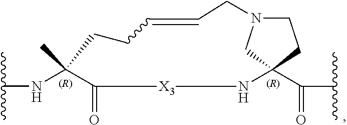

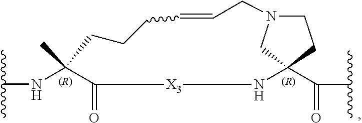

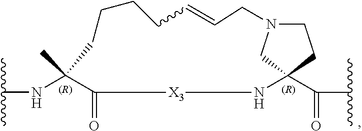

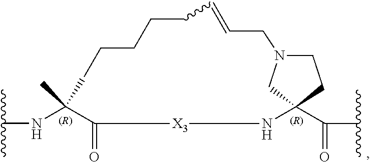

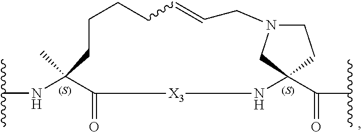

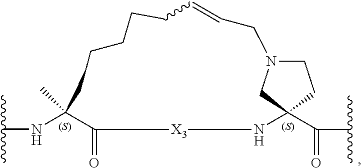

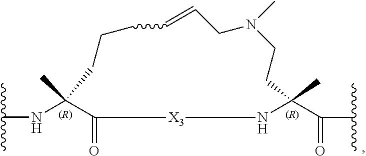

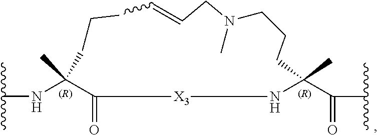

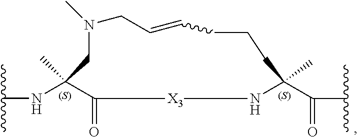

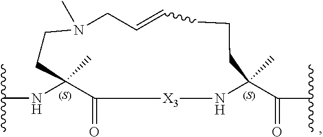

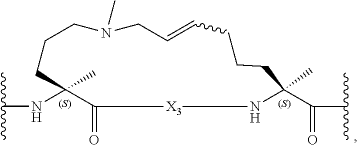

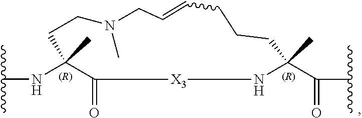

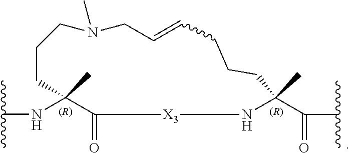

TABLE S-3
Exemplary staples.

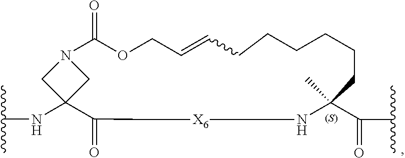

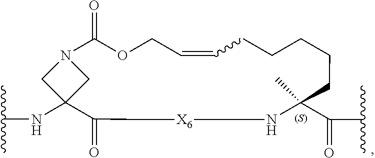

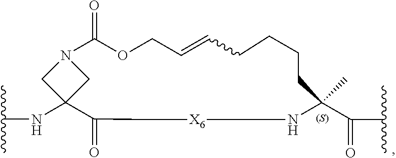

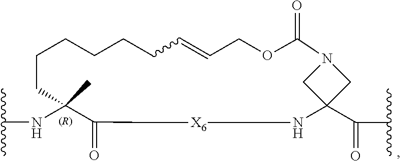

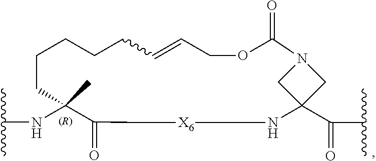

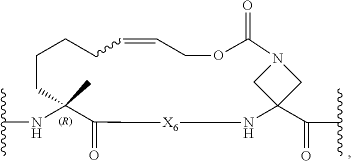

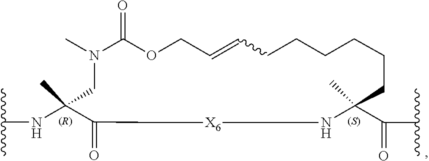

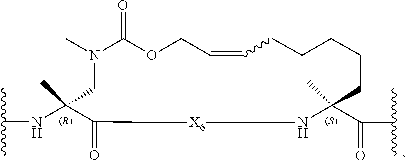

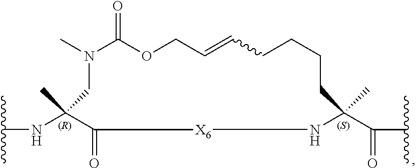

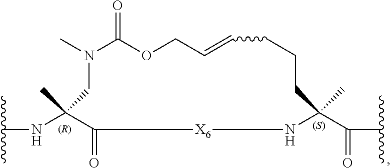

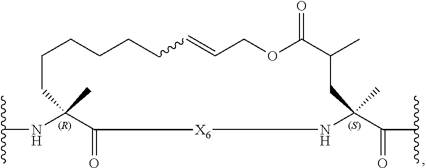

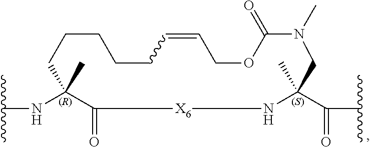

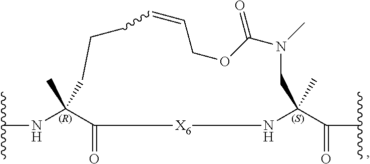

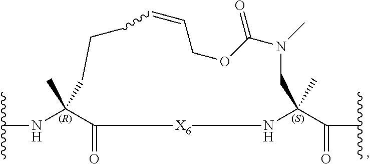

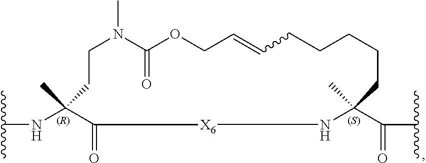

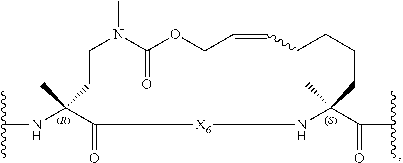

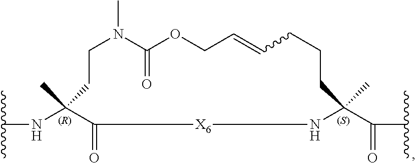

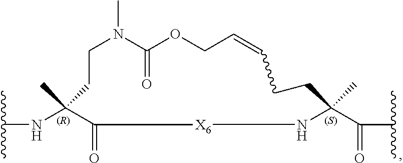

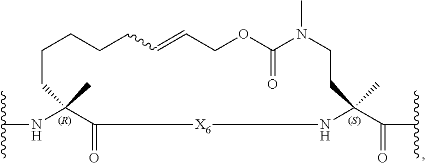

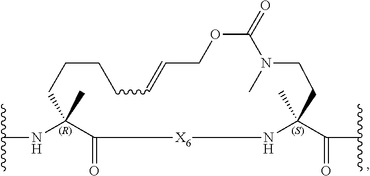

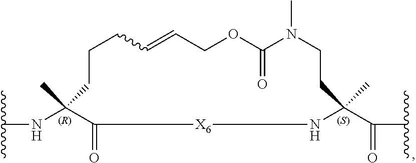

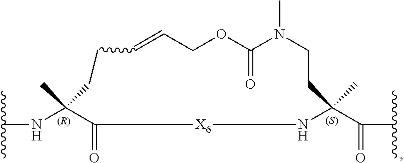

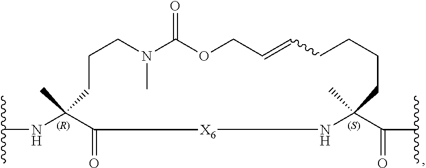

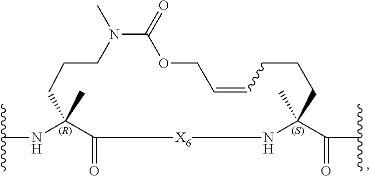

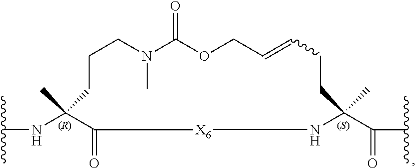

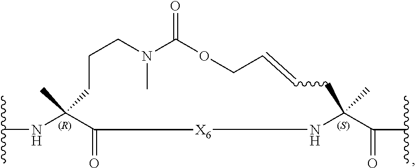

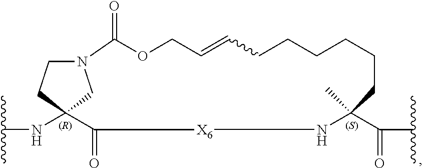

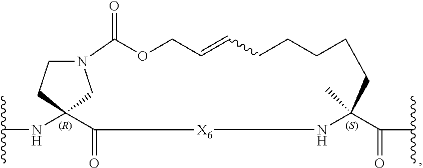

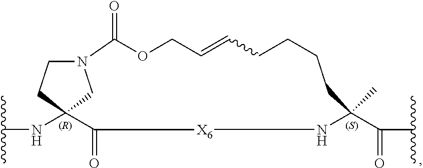

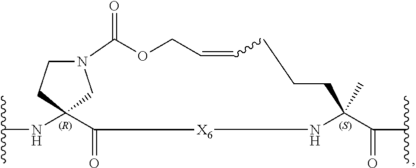

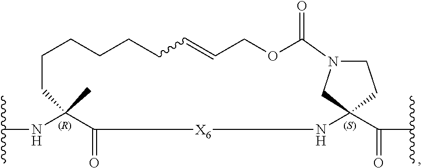

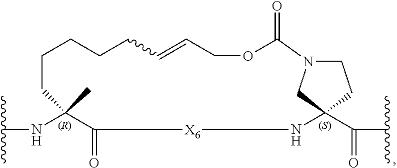

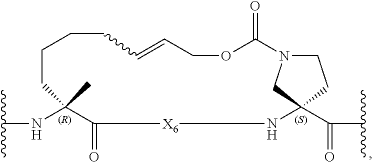

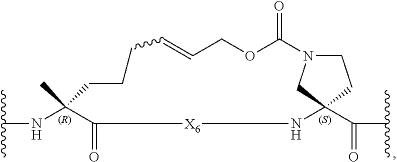

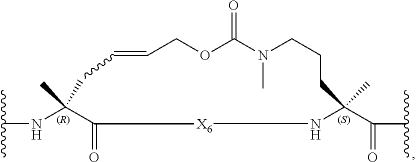

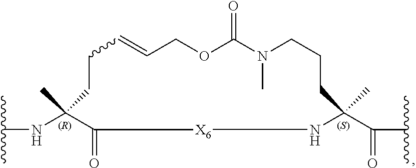

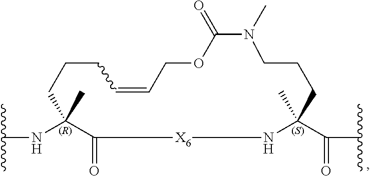

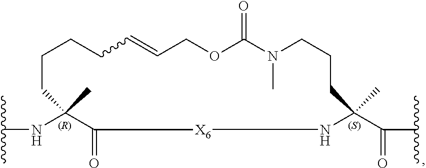

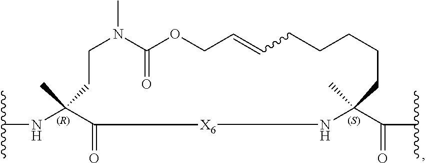

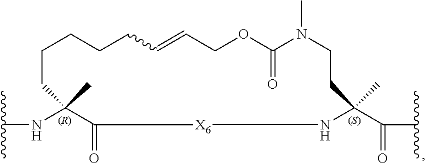

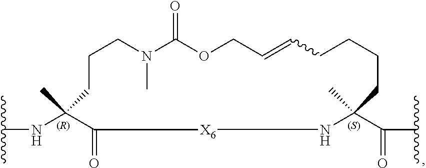

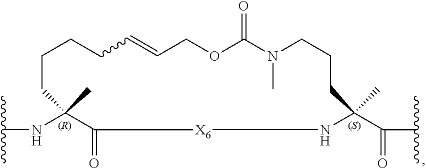

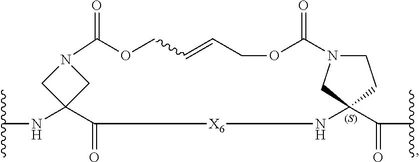

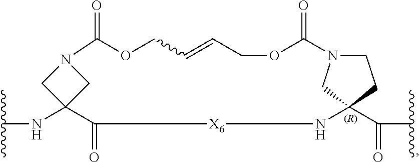

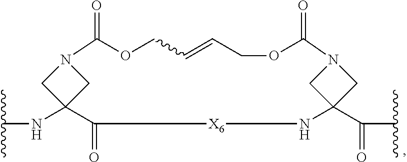

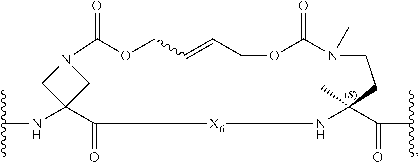

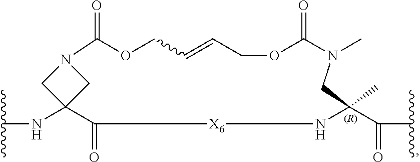

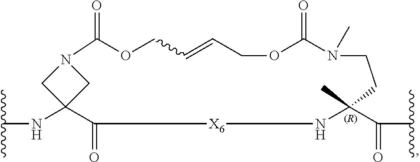

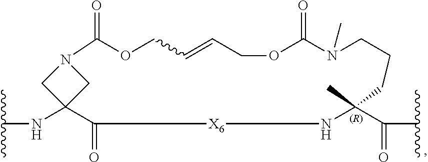

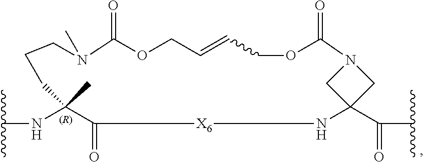

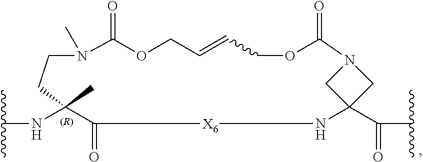

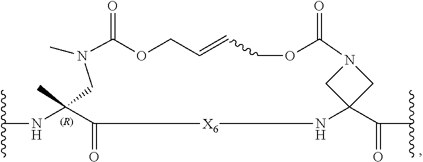

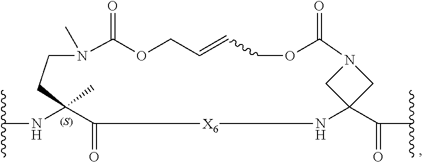

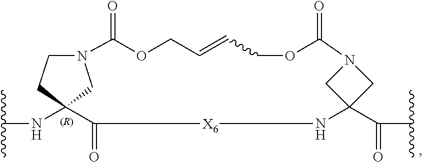

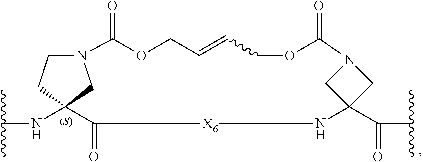

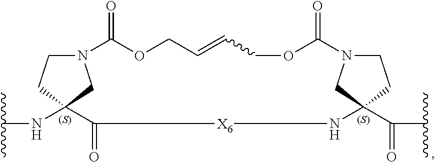

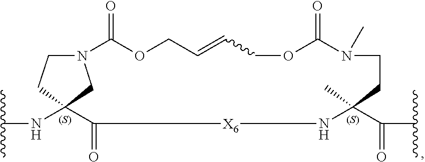

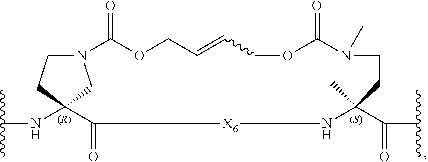

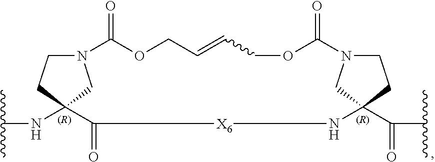

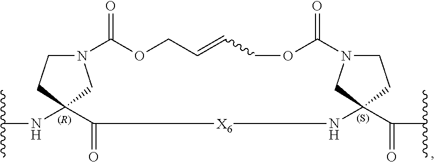

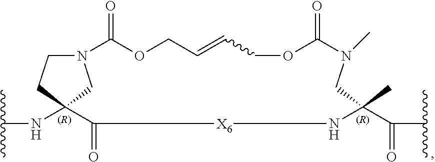

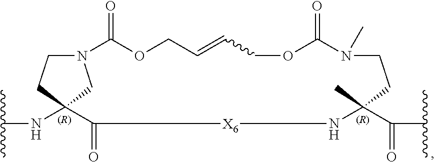

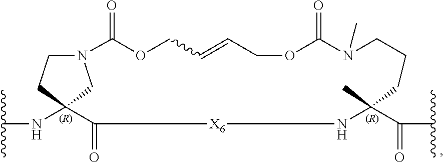

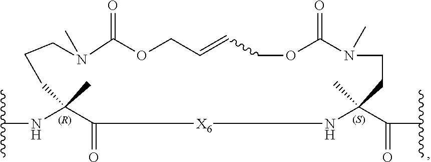

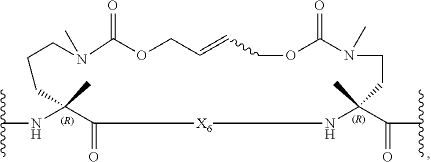

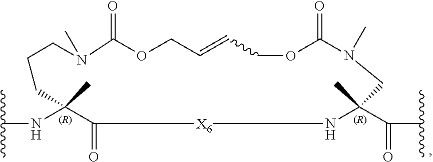

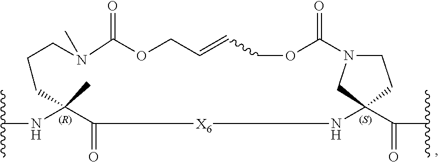

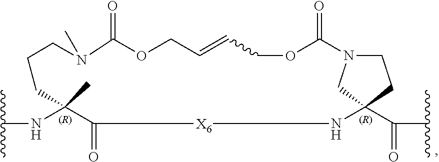

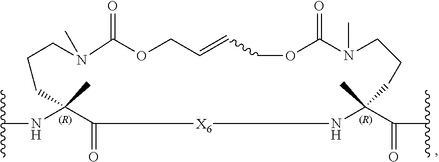

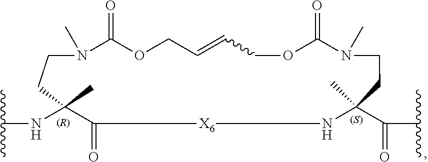

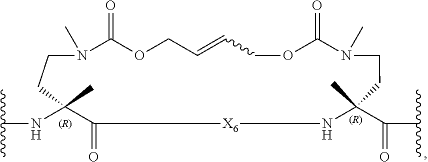

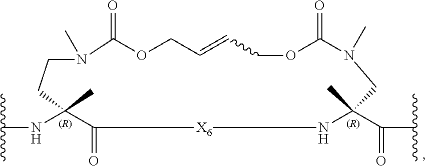

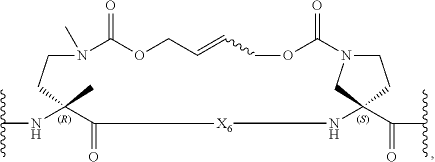

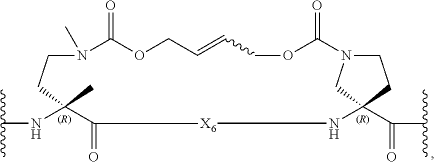

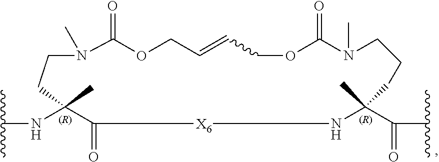

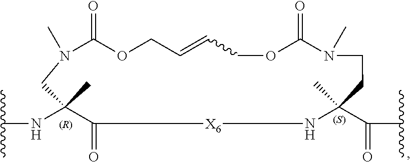

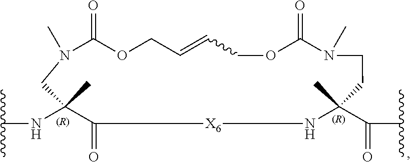

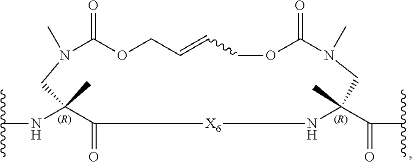

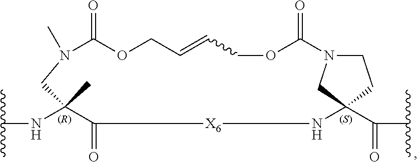

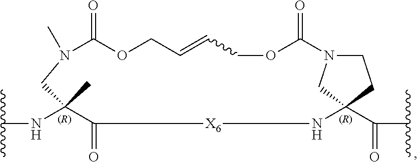

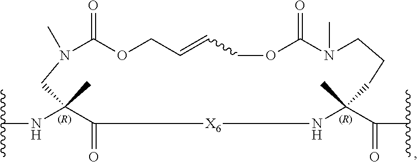

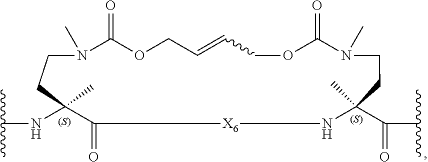

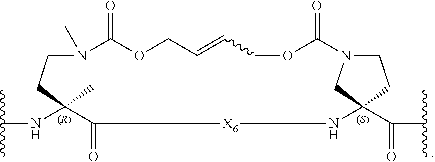

TABLE S-4
Exemplary staples.

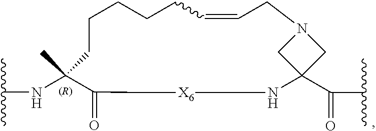

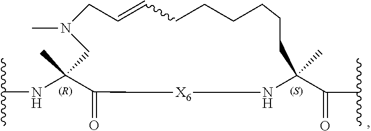

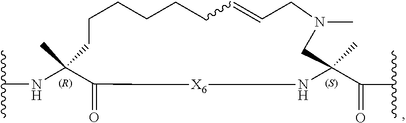

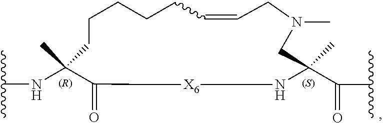

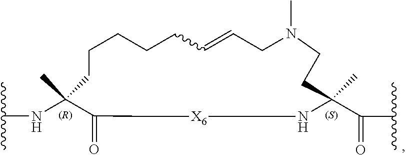

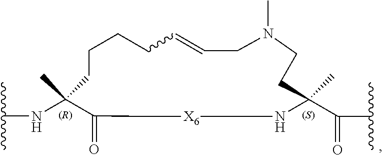

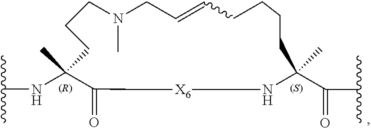

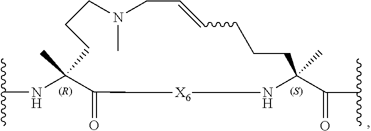

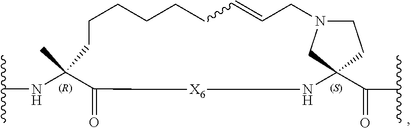

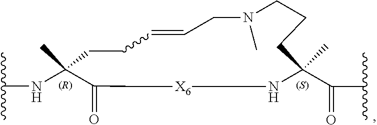

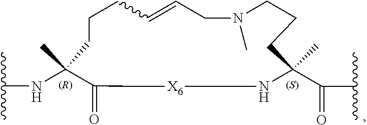

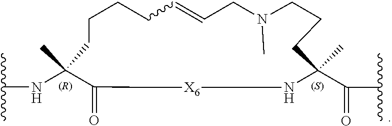

As described herein, in various instances, a group is or comprises an optionally substituted ring. For example, in some embodiments, R or a group that can be R (e.g., R′) is or comprises an optionally substituted ring as described herein. In some embodiments, two or more R groups, or two or more groups that are or can be R (e.g., R′, R², R³, etc.,), can be taken together with their intervening atom(s) to form an optionally substituted ring as described herein. In some embodiments, a ring is substituted (in addition to groups attached to the intervening atom(s)). In some embodiments, a ring is unsubstituted. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. In some embodiments, a ring is saturated. In some embodiments, a ring is partially unsaturated. In some embodiments, a ring is aromatic. In some embodiments, a ring is monocyclic. In some embodiments, it is bicyclic. In some embodiments, it is polycyclic. In some embodiments, each monocyclic unit is independently a 3-10 (e.g., 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4) heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a ring has, in addition to the intervening atom(s), 0-4 (e.g., 0, 1, 2, 3, or 4, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, R or a group that can be R has at least one but no more than about 10 (e.g., 1-9, 1-8, 1-6, 1, 2, 3, 4, 5, 6, 7, 8, 9, 10, etc.) carbon atoms and no more than 6 (e.g., 0-5, 1-5, 1, 2, 3, 4, 5, 6, etc.) heteroatoms independently selected from nitrogen, oxygen and sulfur. In some embodiments, a bivalent moiety, e.g., L′, L, etc., has no more than about 10 (e.g., 1-9, 1-8, 1-6, 1, 2, 3, 4, 5, 6, 7, 8, 9, 10, etc.) carbon atoms and no more than 6 (e.g., 0-5, 1-5, 1, 2, 3, 4, 5, 6, etc.) heteroatoms independently selected from nitrogen, oxygen and sulfur.

As described herein, various groups may be optionally substituted. Substituents are routinely utilized in chemistry including in development of various therapeutics. Many substituents can be utilized in accordance with the present disclosure. In some embodiments, an optionally substituted group is unsubstituted. In some embodiments, an optionally substituted group is substituted. Substituents are preferably those that result in the formation of compounds for a desired property, activity, use, etc., as described herein. In some embodiments, compounds are stable for therapeutic use as described herein. The term “stable,” as used herein, refers to compounds that are not substantially altered when subjected to conditions to allow for their production, detection, and, in certain embodiments, their recovery, purification, and use for one or more of the purposes disclosed herein. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is no more than about 2; in some embodiments, it is no more than about 3; in some embodiments, it is no more than about 4; in some embodiments, it is no more than about 5; in some embodiments, it is no more than about 6; in some embodiments, it is no more than about 7; in some embodiments, it is no more than about 8; in some embodiments, it is no more than about 9; in some embodiments, it is no more than about 10; in some embodiments, it is no more than about 11; in some embodiments, it is no more than about 12; in some embodiments, it is no more than about 13; in some embodiments, it is no more than about 14; in some embodiments, it is no more than about 15; in some embodiments, it is no more than about 20. In some embodiments, the total number of carbon and non-halogen heteroatom(s) in each substituent is independently no more than about 20. In some embodiments, the total number of carbon and non-halogen heteroatom(s) in each substituent is independently no more than about 15. In some embodiments, the total number of carbon and non-halogen heteroatom(s) in each substituent is independently no more than about 10. In some embodiments, the total number of carbon and non-halogen heteroatom(s) in each substituent is independently no more than about 6. In some embodiments, each optional substituent on a substitutable group is independently halogen, C₁₋₄ alkyl, —OH, —CN, —NO₂, C₁₋₄ haloalkyl (e.g., —CF₃), —OR^{SB}, —N(R^{SB})₂, —C(O)OR^{SB}, —C(O)N(R^{SB})₂, or —S(O)₂N(R^{SB})₂ wherein each R^{SB} is independently —H, C₁₋₄ alkyl or C₁₋₄ haloalkyl. In some embodiments, each optional substituent on a substitutable group is independently halogen, C₁₋₄ alkyl, C₁₋₄ haloalkyl, or —OH. In some embodiments, each optional substituent on a substitutable group is independently halogen or C₁₋₄ alkyl.

### Peptide Characterization

In some embodiments, peptides as described herein (e.g., stapled peptides, e.g., cysteine stapled peptides, non-cysteine stapled peptides that are variants of cysteine stapled peptides, as described herein, and/or collections thereof) are characterized with respect to, for example, one or more characteristics selected from the group consisting of: binding characteristics—e.g., with respect to a particular target of interest; stability characteristics, for example in solution or in dried form; cell permeability characteristics, etc., and combinations thereof.

In some embodiments, a binding characteristic may be or comprise specificity, affinity, on-rate, off-rate, etc, optionally under (or over a range of) specified conditions such as, for example, concentration, temperature, pH, cell type, presence or level of a particular competitor, etc.

As will be appreciated by those skilled in the art, assessments of characteristics as described herein may involve comparison with an appropriate reference (e.g., a positive or negative control) which may, in some embodiments, be a contemporaneous reference or, in some embodiments, a historical reference.

In some embodiments, desirable peptide characteristics may be, for example: binding to a target of interest (e.g., binding affinity of about or no more than about 1, 2, 3, 4, 5, 6, 7, 8, 9, 10, 15, 20, 25, 30, 40, 50, 60, 70, 80, 90 or 100 μM, and preferably no more than 10, 1 0.5, 0.1, 0.05 or 0.01 μM); cell penetration (e.g., as measured by fluorescence-based assays or mass spectrometry of cellular fractions, etc.); activity (e.g., modulating one or more functions of a target, which may be assessed in a cellular reporter assay (e.g., with an IC50 of no more than a concentration, e.g., about 10, 1 μM, 500 nM, etc.), an animal model and/or a subject; stability, which may be assessed using a number of assays (e.g., in a rat pharmacokinetic study (e.g., administered via oral, iv, ip, etc.) with a terminal half-life of greater than a suitable time, e.g., 1 hour); low toxicity, which might be assessed by a number of assays (e.g., a standard ADME/toxicity assays); and/or low levels of cytotoxicity (e.g., low levels of lactate dehydrogenase (LDH) released from cells when treated at a suitable concentration, e.g., about 10 μM of a peptide).

### Peptide Production

Various technologies are known in the art for producing stapled peptides of may be utilized in accordance with the present disclosure. Those skilled in the art, reading the present disclosure, will well appreciate which such technologies are applicable in which aspects of the present disclosure. Certain technologies are described U.S. Ser. No. 11/198,713, US 20210179665, WO 2021119537, WO 2021188659, WO 2022020651, or WO 2022020652, the entirety of each of which is incorporated herein by reference and can be utilized in accordance with the present disclosure.

In some embodiments, as described herein, certain stapled peptides, and in particular cysteine stapled peptides, may be provided in and/or produced by a biological system and reacting with a provided reagent, e.g., one having the structure of R^{x}-L^{s2}-R^{x} formula R-I, or a salt thereof.

In some embodiments, peptides are prepared on solid phase on a synthesizer using, typically, Fmoc chemistry.

In some embodiments, staples are formed by olefin metathesis. In some embodiments, a product double bond of metathesis is reduced/hydrogenated. In some embodiments, CO₂ are extruded from a carbamate moiety of a staple. In some embodiments, provided stapled peptides are further modified, and/or conjugated to other entities. Conditions and/or reagents of these reactions are widely known in the art and can be performed in accordance with the present disclosure to provide stapled peptides.

Properties and/or activities of provided stapled peptides can be readily assessed in accordance with the present disclosure, for example, through use of one or more methods described in the examples.

In some embodiments, technologies for preparing and/or assessing provided stapled peptides include those described in U.S. Pat. No. 9,617,309, US 2015-0225471, US 2016-0024153, US 2016-0215036, US2016-0244494, WO 2017/062518, etc.

In some embodiments, a provided agent, e.g., a provided peptide, has a purity of 60%-100%. In some embodiments, a provided agent has a purity of at least 60%, 65%, 70%, 75%, 80%, 85%, 90%, 91%, 92%, 93%, 94%, 95%, 96%, 97%, 98%, or 99%. In some embodiments, a purity is at least 60%. In some embodiments, a purity is at least 70%. In some embodiments, a purity is at least 80%. In some embodiments, a purity is at least 85%. In some embodiments, a purity is at least 90%. In some embodiments, a purity is at least 91%. In some embodiments, a purity is at least 92%. In some embodiments, a purity is at least 93%. In some embodiments, a purity is at least 94%. In some embodiments, a purity is at least 95%. In some embodiments, a purity is at least 96%. In some embodiments, a purity is at least 97%. In some embodiments, a purity is at least 98%. In some embodiments, a purity is at least 99%. In some embodiments, a purity is at least 99.5%.

In some embodiments, provided methods provide high yields. In some embodiments, a yield is 50%-100%. In some embodiments, a yield is at least 60%, 65%, 70%, 75%, 80%, 85%, 90%, 91%, 92%, 93%, 94%, 95%, 96%, 97%, 98% or 99%. In some embodiments, a yield is at least 60%. In some embodiments, a yield is at least 65%. In some embodiments, a yield is at least 70%. In some embodiments, a yield is at least 75%. In some embodiments, a yield is at least 80%. In some embodiments, a yield is at least 85%. In some embodiments, a yield is at least 90%. In some embodiments, a yield is at least 91%. In some embodiments, a yield is at least 92%. In some embodiments, a yield is at least 93%. In some embodiments, a yield is at least 94%. In some embodiments, a yield is at least 95%. In some embodiments, a yield is at least 96%. In some embodiments, a yield is at least 97%. In some embodiments, a yield is at least 98%. In some embodiments, a yield is at least 99%.

In some embodiments, a provided method delivers high E/Z selectivity for olefin. In some embodiments, provided selectivity favors the E isomer. In some embodiments, provided selectivity favors the Z isomer. In some embodiments, a E:Z ratio is at least 1:1, 1.5:1, 2:1, 3:1, 4:1, 5:1, 6:1, 7:1, 8:1, 9:1, 10:1, 20:1, 30:1, 40:1, 50:1, or 100:1. In some embodiments, a Z:E ratio is at least 1:1, 1.5:1, 2:1, 3:1, 4:1, 5:1, 6:1, 7:1, 8:1, 9:1, 10:1, 20:1, 30:1, 40:1, 50:1, 80:1, 90:1, 95:1, 99:1, or 100:1. In some embodiments, a ratio is at least 1:1. In some embodiments, a ratio is at least 1.5:1. In some embodiments, a ratio is at least 2:1. In some embodiments, a ratio is at least 3:1. In some embodiments, a ratio is at least 4:1. In some embodiments, a ratio is at least 5:1. In some embodiments, a ratio is at least 6:1. In some embodiments, a ratio is at least 7:1. In some embodiments, a ratio is at least 8:1. In some embodiments, a ratio is at least 9:1. In some embodiments, a ratio is at least 10:1. In some embodiments, a ratio is at least 20:1. In some embodiments, a ratio is at least 30:1. In some embodiments, a ratio is at least 40:1. In some embodiments, a ratio is at least 50:1. In some embodiments, a ratio is at least 80:1. In some embodiments, a ratio is at least 90:1. In some embodiments, a ratio is at least 95:1. In some embodiments, a ratio is at least 99:1. In some embodiments, a ratio is at least 100:1.

In some embodiments, a provide method comprises a period of time at a temperature higher than room temperature. In some embodiments, a temperature is about 25-200° C. In some embodiments, a temperature is about 25° C. In some embodiments, a temperature is about 30° C. In some embodiments, a temperature is about 35° C. In some embodiments, a temperature is about 40° C. In some embodiments, a temperature is about 45° C. In some embodiments, a temperature is about 50° C. In some embodiments, a temperature is about 55° C. In some embodiments, a temperature is about 60° C. In some embodiments, a temperature is about 65° C. In some embodiments, a temperature is about 70° C. In some embodiments, a temperature is about 75° C. In some embodiments, a temperature is about 80° C. In some embodiments, a temperature is about 85° C. In some embodiments, a temperature is about 90° C. In some embodiments, a temperature is about 95° C. In some embodiments, a temperature is about 100° C. In some embodiments, a temperature is about 150° C. In some embodiments, a temperature is higher than about 150° C.

### Peptide Compositions

Among other things, the present disclosure provides compositions that comprise or otherwise relate to peptides, e.g., stapled peptides, as described herein.

For example, in some embodiments, provided compositions are or comprise elements of a phage display system that encodes and/or expresses stapled peptides (e.g., cysteine stapled peptides), or a collection thereof, as described herein.

In some embodiments, provided compositions are or comprise an assay system for characterizing (and optionally including) a stapled peptide as described herein.

In some embodiments, provided compositions are pharmaceutical compositions e.g., that comprise or deliver one or more stapled peptides (e.g., in particular one or more non-cysteine stapled peptides that may, in some embodiments, correspond to and/or be a variant of a parent cysteine stapled peptide as described herein).

In some embodiments, a pharmaceutical composition comprises a peptide agent and a pharmaceutically acceptable carrier.

In some embodiments, a peptide composition may include or deliver a particular form (e.g., a particular optical isomer, diastereomer, salt form, covalent conjugate form [e.g., covalently attached to a carrier moiety], etc., or combination thereof) of a peptide agent as described herein). In some embodiments, a peptide agent included or delivered by a pharmaceutical composition is described herein is not covalently linked to a carrier moiety.

In some embodiments, a provided therapeutic composition may comprise one or more additional therapeutic agents and/or one or more stabilizing agents and/or one or more agents that alters (e.g., extends or limits to a particular tissue, location or site) rate or extent of delivery over time.

### Uses and Applications

In some embodiments, the present disclosure provides certain stapled peptides and/or other technologies (e.g., collections of stapled peptides, and/or a biological system adapted to express or display and/or expressing or displaying such stapled peptide(s), etc.) for the development and/or use of stapled peptides (e.g., that bind a target of interest).

In some embodiments, the present disclosure provides collections of peptides, wherein the collection of peptides comprise one or more (e.g., about or at least about 1, 2, 3, 4, 5, 6, 7, 8, 9, or 10) degenerate positions and two or more residues stapled or suitable for stapling. In some embodiments, two or more residues are stapled. In some embodiments, two or more residues are suitable for stapling and can be stapled in accordance with the present disclosure. In some embodiments, a collection of peptides comprises one or more positions at each of which a single amino acid or a selected set of amino acids are utilized. In some embodiments, a collection of peptides comprises a set of enriched amino acid residues at a position (an enriched position). In some embodiments, a collection of peptides comprises a set of enriched amino acid residues independently at two or more enriched positions. In some embodiments, a set of enriched amino acid residues comprise about or at least about 1, 2, 3, 4, 5, 6, 7, 8, 9, or 10 unique amino acid residues. In some embodiments, there is a single amino acid residue in a set. In some embodiments, there are two or more amino acid residues in a set. In some embodiments, compared to at a degenerate position, there are fewer unique amino acid residues independently at each enriched position. In some embodiments, a set of enriched amino acid residues is selected for interacting with a target of interest, e.g., in some embodiments, a first target of interest. In some embodiments, a set of enriched amino acid residues occurs at higher frequency in stapled peptides binding to a target of interest compared to stapled peptides that do not bind to a target of interest, or an initial collection of stapled peptides for screening.

In some embodiments, the present disclosure provides certain stapled peptides, or collections thereof (e.g., including collections in which individual peptides may be fused with a phage coat protein), and/or a biological system adapted to express or display and/or expressing or displaying such cysteine stapled peptide(s). In some embodiments, certain provided stapled peptides and/or other technologies can be utilized to identify and/or characterize one or more desirable structural features (e.g., amino acid sequence, staple location and/or structure [e.g., length, composition, degree of constraint, etc.]) of a stapled peptide that interacts with (e.g., binds to) a target of interest. In some embodiments, certain provided stapled peptides and/or other technologies are useful for the development of stapled peptides (e.g., non-cysteine stapled peptides) that correspond to (e.g., share significant structural identity with, and optionally structural identity with, a reference cysteine stapled peptide, except for substitution of the cysteine(s) for other non-cysteine staple-forming residue(s)).

In some embodiments, a provided peptide or collection thereof, whose amino acid sequence, includes at least two appropriately spaced cysteine residues, is used to prepare a cysteine stapled peptide, or collection thereof (e.g., by reaction with a compound of formula R-I. certain provided stapled peptides and/or other technologies

In some embodiments, certain provided stapled peptides can be identified and synthesized by technologies and examples described in the present disclosure. In some embodiments, a certain provided stapled peptide is a cysteine stapled peptide. In some embodiments, a cysteine stapled peptide that binds a target of interest may have a cysteine staple and corresponding cysteine residues replaced by a non-cysteine staple and amino acids necessary to facilitate such a non-cysteine staple.

In some embodiments, a provided collection of peptides (or nucleic acids that encode them) is characterized in that peptides of the collection all include cysteine residues (e.g. a pair of cysteine residues), spaced relative to one another to permit cysteine stapling as described herein, but otherwise have independent amino acid sequences and, optionally, in that peptides of the collection all have the same length. In some embodiments, degeneracy and/or bias is introduced in one or more positions through genetic engineering and/or expression of selected nucleic acid sequences in a biological system. In some embodiments, degree of degeneracy or bias at one or more positions in peptides of a peptide collection or library as described here is informed and/or selected by prior assessment one or more binding characteristics of a related library or collection (e.g., with comparable cysteine residues). In some embodiments, such prior assessment is by high-throughput analysis (e.g., screening) of a collection or collections of stapled peptides against a target of interest and the use of high-throughput sequencing to decode the genotypes of a subset of the collection of stapled peptides can inform the production of a biased library.

In some embodiments, a collection of stapled peptides comprises cysteine stapled peptides fused to another molecule for use in a biological system (e.g. phage display) or non-biological system. In some embodiments, after screening a collection of stapled peptides in the context of a biological system or non-biological system, high throughput sequencing will identify particular cysteine stapled peptides that interact with a target of interest. In some embodiments, a particular cysteine stapled peptide, when not fused to another molecule for use in a biological system, will exhibit the same or similar interaction with a target of interest as the particular cysteine stapled peptide when fused to said molecule. In some embodiments, a particular cysteine stapled peptide, when not fused to another molecule for use in a biological system, can have its cysteine residues and cysteine staple replaced with other amino acids and staples. In some embodiments, the resulting peptide maintains the same or similar interaction with a target of interest.

In some embodiments, a collection of stapled peptides comprises non-cysteine staples.

In some embodiments, certain provided stapled peptides and/or other technologies as described herein may be useful to modulate one or more biological events or statuses, e.g., by binding with a relevant target of interest.

In some embodiments, the present disclosure provides a method for modulating an activity of a target of interest, comprising contacting the target of interest with an agent described herein. In some embodiments, the present disclosure provides a method for modulating interaction of a target of interest with a partner, comprising contacting the target of interest with an agent described herein. In some embodiments, the present disclosure provides a method for modulating an activity of a target of interest in a system comprising the target of interest, comprising administering or delivering to the system an agent described herein. In some embodiments, the present disclosure provides a method for modulating interaction a target of interest with a partner in a system comprising the target of interest, comprising administering or delivering to the system an agent described herein. In some embodiments, a system expresses a target of interest. In some embodiments, a system is in vivo. In some embodiments, a system is in vitro. In some embodiments, a system is or comprises a cell. In some embodiments, a system is or comprises a population of cells. In some embodiments, a system is or comprises a tissue. In some embodiments, a system is or comprises an organ. In some embodiments, a system is a subject. In some embodiments, a partner is a polypeptide. In some embodiments, a partner is a protein. In some embodiments, an activity is inhibited. In some embodiments, an interaction is reduced. In some embodiments, an interaction is enhanced. In some embodiments, an agent comprises a stapled peptide. In some embodiments, an agent is a stapled peptide.

In some embodiments, provided technologies enables de novo design of stapled peptides for targets of interest including protein targets without prior information on their alpha-helix binding properties, which has significantly limited the proteins and diseases for which stapled peptides or other alpha-helical structure could be discovered. In some embodiments, provided technologies can be utilized to identify, characterize, and produce stapled peptides that can bind to and modulate activities of various targets of interest, including many that have not been reported to bind to isolated a-helical peptides. In some embodiments, provided technologies provide distinct a-helix recognition sites. Among other things, provided technologies can block protein-protein interactions, inhibit enzymatic activity, induce conformational rearrangements, and cause protein dimerization.

Certain agents, collections, methods, etc. are described below as examples. In some embodiments, at a position where there is no strong preference or enrichment, an amino acid residue, e.g., X (such as X³¹, X², etc. if no enrichment), the amino acid is selected from natural amino acids. In some embodiments, it is selected from alanine, arginine, asparagine, aspartic acid, glutamine, glutamic acid, histidine, isoleucine, leucine, methionine, phenylalanine, serine, threonine, tryptophan, tyrosine, and valine.

In some embodiments, a sequence may be truncated. In some embodiments, a truncated sequence may be part of optimization, e.g., to increase affinity. In some embodiments, one or more (e.g., 1, 2, or 3) or all residues to the N-terminus side of a stapled residue (e.g., X⁴), e.g., X³¹, X³², X³³, X³⁴, X³⁵, X⁴¹, X⁴², X⁴³, X⁴⁴, X⁴⁵, X⁴⁶, X⁴⁷, X⁴⁸, X⁵¹, X⁵², X⁵³, X⁵⁴, X⁶¹, X⁶², X², X³, etc. are absent. In some embodiments, one or more (e.g., 1, 2, or 3) or all residues to the C-terminus side of a stapled residue (e.g., X¹¹), e.g., X¹², X¹³, X¹⁴, etc. are absent. In some embodiments, X³¹, X³², X³³, X³⁴, X³⁵, X⁴¹, X⁴², X⁴³, X⁴⁴, X⁴⁵, X⁴⁶, X⁴⁷, X⁴⁸, X⁵¹, X⁵², X⁵³, X⁵⁴, X⁶¹, or X⁶² is absent. In some embodiments, X² is absent. In some embodiments, X³ is absent. In some embodiments, X¹² is absent. In some embodiments, X¹³ is absent. In some embodiments, X¹⁴ is absent.

### Beta-Catenin

In some embodiments, the present disclosure provides agents that can bind to and/or modulate activities of beta-catenin. In some embodiments, stapled peptides comprising sequences of certain clusters, e.g., cluster 31, cluster 32, cluster 33 and cluster 43, compete with axin for beta-catenin interaction. In some embodiments, stapled peptides comprising sequences of certain clusters, e.g., cluster 31, cluster 32, cluster 33 and cluster 43, compete with TCF for beta-catenin interaction. In some embodiments, stapled peptides comprising sequences of certain clusters, e.g., cluster 31, cluster 32, cluster 33 and cluster 43, compete with both axin and TCF for beta-catenin interaction. In some embodiments, stapled peptides comprising sequences of certain clusters, e.g., cluster 35, compete with TCF for beta-catenin interaction. In some embodiments, stapled peptides comprising sequences of certain clusters, e.g., cluster 35, compete with TCF but not axin for beta-catenin interaction.

### Cluster C31

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C31 as described herein. In some embodiments, an agent comprises X³¹X²X³X⁴X⁵X⁶X⁷X⁸X⁹X¹⁰X¹¹X¹²X¹³X¹⁴, wherein each of X³¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³¹X²X³X⁴X⁵X⁶X⁷X⁸X⁹X¹⁰X¹¹X¹²X¹³X¹⁴,

wherein:
each of X³¹, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising an acidic group;
X⁵ comprises a hydrophobic side chain;
X⁶ comprises a hydrophobic side chain;
X⁹ comprises a side chain comprising an aromatic group or a basic group; and
X¹⁰ comprises a hydrophobic side chain.

Various amino acid residues may be utilized for X³¹. For example, in some embodiments, X³¹ comprises a side chain comprising an acidic group. In some embodiments, a cluster is enriched for amino acid residues whose side chains comprising acidic groups at X³¹. In some embodiments, X³¹ comprises a side chain comprising —COOH. In some embodiments, X³¹ is D. In some embodiments, X³¹ is E. In some embodiments, X³¹ comprises a hydrophobic side chain. In some embodiments, X³¹ is V. In some embodiments, X³¹ comprises a side chain comprising a polar group. In some embodiments, X³¹ is Q. In some embodiments, X³¹ is N.

Various amino acid residues may be utilized for X². For example, in some embodiments, X² comprises a side chain comprising an acidic group. In some embodiments, X² comprises a side chain comprising —COOH. In some embodiments, X² is D. In some embodiments, X² is E. In some embodiments, X² comprises a hydrophobic side chain. In some embodiments, X² is A. In some embodiments, X² is V. In some embodiments, X² comprises a side chain comprising a polar group. In some embodiments, X² is Q. In some embodiments, X² is N.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, X³ comprises a hydrophobic side chain. In some embodiments, X³ is I. In some embodiments, X³ comprises a side chain comprising a polar group. In some embodiments, X³ is Q. In some embodiments, X³ is N. In some embodiments, X³ comprises a side chain comprising —OH. In some embodiments, X³ is S. In some embodiments, X³ is T.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

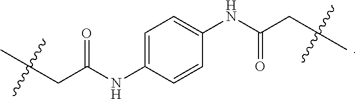 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is I. In some embodiments, X⁵ is V. In some embodiments, X⁵ is M. In some embodiments, X⁵ comprises a side chain comprising a polar group. In some embodiments, a cluster is enriched for amino acid residues comprising a polar group at X⁵. In some embodiments, X⁵ comprises a side chain comprising —OH. In some embodiments, X⁵ is T.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁ 6alkyl. In some embodiments, X⁶ is L. In some embodiments, X⁶ is M. In some embodiments, X⁶ is I.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising a basic group. In some embodiments, a cluster is enriched for amino acid residues comprising a basic group at X⁹. In some embodiments, X⁹ is H. In some embodiments, X⁹ comprises a side chain comprising a polar group. In some embodiments, X⁹ comprises a side chain comprising —OH. In some embodiments, X⁹ is Y. In some embodiments, X⁹ is Q.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C_{1 6} aliphatic. In some embodiments, the side chain of X¹⁰ is C₁₋₆ alkyl. In some embodiments, the side chain of X¹⁰ comprises an aromatic group. In some embodiments, X¹⁰ is I. In some embodiments, X¹⁰ is V. In some embodiments, X¹⁰ is F. In some embodiments, X¹⁰ comprises a side chain comprising an acidic group. In some embodiments, a cluster is enriched for amino acid residues comprising an acidic group at X¹⁰. In some embodiments, X¹⁰ comprises a side chain comprising —COOH. In some embodiments, X¹⁰ is E.

Various amino acid residues may be utilized for X¹². For example, in some embodiments, X¹² comprises a side chain comprising a basic group. In some embodiments, a cluster is enriched for amino acid residues comprising a basic group at X¹². In some embodiments, X¹² is R. In some embodiments, X¹² is H. In some embodiments, X¹² comprises a side chain comprising a polar group. In some embodiments, a cluster is enriched for amino acid residues comprising a polar group at X¹². In some embodiments, X¹² is Q. In some embodiments, X¹² is N. In some embodiments, X¹² is A.

Various amino acid residues may be utilized for X¹³. For example, in some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, the side chain of X¹³ comprises an aromatic group. In some embodiments, X¹³ is I. In some embodiments, X¹³ is L. In some embodiments, X¹³ is A. In some embodiments, X¹⁰ is V. In some embodiments, X¹⁰ is W. In some embodiments, X¹³ comprises a side chain comprising a basic group. In some embodiments, a cluster is enriched for amino acid residues comprising a basic group at X¹³. In some embodiments, X¹³ is H. In some embodiments, X¹³ comprises a side chain comprising a polar group. In some embodiments, X¹³ is N.

Various amino acid residues may be utilized for X¹⁴. For example, in some embodiments, X¹⁴ comprises a hydrophobic side chain. In some embodiments, a cluster is enriched for amino acid residues comprising a hydrophobic side chain at X¹⁴. In some embodiments, the side chain of X¹⁴ is C₁₋₆ aliphatic. In some embodiments, the side chain of X¹⁴ is C₁₋₆ alkyl. In some embodiments, the side chain of X¹⁴ comprises an aromatic group. In some embodiments, X¹⁴ is I. In some embodiments, X¹⁴ is F. In some embodiments, X¹⁴ is W. In some embodiments, X¹⁴ comprises a side chain comprising an acidic group. In some embodiments, a cluster is enriched for amino acid residues whose side chains comprising acidic groups at X¹⁴. In some embodiments, X¹⁴ comprises a side chain comprising —COOH. In some embodiments, X¹⁴ is D. In some embodiments, X¹⁴ is E.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of SEQ ID NO: 1 or amino acid residues corresponding thereto: H219, H223, S250, F253, Y254, T257, N261, H260, L264, K292, A295, I296, N299, Y333, K335, W338, and T339. In some embodiments, it interacts with H219 of SEQ ID NO: 1 or an amino acid residue corresponding thereto. In some embodiments, it interacts with H223 of SEQ ID NO: 1 or an amino acid residue corresponding thereto. In some embodiments, it interacts with S250 of SEQ ID NO: 1 or an amino acid residue corresponding thereto. In some embodiments, it interacts with F253 of SEQ ID NO: 1 or an amino acid residue corresponding thereto. In some embodiments, it interacts with Y254 of SEQ ID NO: 1 or an amino acid residue corresponding thereto. In some embodiments, it interacts with T257 of SEQ ID NO: 1 or an amino acid residue corresponding thereto. In some embodiments, it interacts with N261 of SEQ ID NO: 1 or an amino acid residue corresponding thereto. In some embodiments, it interacts with H260 of SEQ ID NO: 1 or an amino acid residue corresponding thereto. In some embodiments, it interacts with L264 of SEQ ID NO: 1 or an amino acid residue corresponding thereto. In some embodiments, it interacts with K292 of SEQ ID NO: 1 or an amino acid residue corresponding thereto. In some embodiments, it interacts with A295 of SEQ ID NO: 1 or an amino acid residue corresponding thereto. In some embodiments, it interacts with I296 of SEQ ID NO: 1 or an amino acid residue corresponding thereto. In some embodiments, it interacts with N299 of SEQ ID NO: 1 or an amino acid residue corresponding thereto. In some embodiments, it interacts with Y333 of SEQ ID NO: 1 or an amino acid residue corresponding thereto. In some embodiments, it interacts with K335 of SEQ ID NO: 1 or an amino acid residue corresponding thereto. In some embodiments, it interacts with W338 of SEQ ID NO: 1 or an amino acid residue corresponding thereto. In some embodiments, it interacts with T339 of SEQ ID NO: 1 or an amino acid residue corresponding thereto.

(SEQ ID NO: 1)
MATQADLMELDMAMEPDRKAAVSHWQQQSYLDSGIHSGATTTAPSLSGK
GNPEEEDVDTSQVLYEWEQGFSQSFTQEQVADIDGQYAMTRAQRVRAAM
FPETLDEGMQIPSTQFDAAHPTNVQRLAEPSQMLKHAVVNLINYQDDAE
LATRAIPELTKLLNDEDQVVVNKAAVMVHQLSKKEASRHAIMRSPQMVS
AIVRTMQNTNDVETARCTAGTLHNLSHHREGLLAIFKSGGIPALVKMLG
SPVDSVLFYAITTLHNLLLHQEGAKMAVRLAGGLQKMVALLNKTNVKFL
AITTDCLQILAYGNQESKLIILASGGPQALVNIMRTYTYEKLLWTTSRV
LKVLSVCSSNKPAIVEAGGMQALGLHLTDPSQRLVQNCLWTLRNLSDAA
TKQEGMEGLLGTLVQLLGSDDINVVTCAAGILSNLTCNNYKNKMMVCQV
GGIEALVRTVLRAGDREDITEPAICALRHLTSRHQEAEMAQNAVRLHYG
LPVVVKLLHPPSHWPLIKATVGLIRNLALCPANHAPLREQGAIPRLVQL
LVRAHQDTQRRTSMGGTQQQFVEGVRMEEIVEGCTGALHILARDVHNRI
VIRGLNTIPLFVQLLYSPIENIQRVAAGVLCELAQDKEAAEAIEAEGAT
APLTELLHSRNEGVATYAAAVLFRMSEDKPQDYKKRLSVELTSSLFRTE
PMAWNETADLGLDIGAQGEPLGYRQDDPSYRSFHSGGYGQDALGMDPMM
EHEMGGHHPGADYPVDGLPDLGHAQDLMDGLPPGDSNQLAWFDTDL

In some embodiments, an agent comprising a cluster 31 sequence competes with axin for interaction with beta-catenin.

### Cluster C32

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C32 as described herein. In some embodiments, an agent comprises X³²X²X³X⁴X⁵X⁶X⁷X⁸X⁹X¹⁰X¹¹X¹²X¹³X¹⁴, wherein each of X³², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³²X²X³X⁴X⁵X⁶X⁷X⁸X⁹X¹⁰X¹¹X¹²X¹³X¹⁴,

wherein:
each of X³², X², X³, X⁴, X⁵, X⁶, X⁷, X⁸, X⁹, X¹⁰, X¹¹, X¹², X¹³, and X¹⁴ is independently an amino acid residue;
X² comprises a hydrophobic side chain;
X³ comprises a side chain comprising an acidic group;
X⁵ comprises a side chain comprising an aromatic group;
X⁶ comprises a hydrophobic side chain; and
X¹⁰ comprises a side chain comprising an acidic group.

Various amino acid residues may be utilized for X³². For example, in some embodiments, X³² comprises a hydrophobic side chain. In some embodiments, a cluster is enriched for amino acid residues comprising a hydrophobic side chain at X³². In some embodiments, the side chain of X³² is C₁₋₆ aliphatic. In some embodiments, the side chain of X³² is C₁₋₆ alkyl. In some embodiments, the side chain of X³² comprises an aromatic group. In some embodiments, X³² is I. In some embodiments, X³² is V. In some embodiments, X³² is F. In some embodiments, X³² comprises a side chain comprising a polar group. In some embodiments, a cluster is enriched for amino acid residues comprising a polar group at X³². In some embodiments, X³² is T. In some embodiments, X³² comprises a side chain comprising a basic group. In some embodiments, X³² is H.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² is C₁₋₆ aliphatic. In some embodiments, the side chain of X² is C₁₋₆ alkyl. In some embodiments, the side chain of X² comprises an aromatic group. In some embodiments, a cluster is enriched for amino acid residues comprising an aromatic group at X². In some embodiments, X² is L. In some embodiments, X² is F.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, X³ comprises a side chain comprising a polar group. In some embodiments, a cluster is enriched for amino acid residues comprising a polar group at X³. In some embodiments, X³ is A. In some embodiments, X³ is N.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

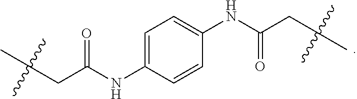 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues comprising a basic group at X⁵. In some embodiments, X⁵ is H. In some embodiments, X⁹ comprises a side chain comprising a polar group. In some embodiments, a cluster is enriched for amino acid residues comprising a polar group at X⁵. In some embodiments, X⁵ is Q. In some embodiments, X⁵ comprises a side chain comprising —OH. In some embodiments, X⁵ is S. In some embodiments, X⁵ is T.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, X⁶ is I. In some embodiments, X⁶ is L. In some embodiments, X⁶ is M. In some embodiments, X⁶ is V.

Various amino acid residues may be utilized for X⁷. In some embodiments, X⁷ is A.

Various amino acid residues may be utilized for X⁷. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C_{1 6} alkyl. In some embodiments, the side chain of X⁹ comprises an aromatic group. In some embodiments, X⁹ is W. In some embodiments, X⁹ is I In some embodiments, X⁹ is L. In some embodiments, X⁹ is M.

Various amino acid residues may be utilized for X¹⁰. For example, in some embodiments, X¹⁰ comprises a side chain comprising an acidic group. In some embodiments, a cluster is enriched for amino acid residues whose side chains comprise acidic groups at X¹⁰. In some embodiments, X¹⁰ comprises a side chain comprising —COOH. In some embodiments, X¹⁰ is D. In some embodiments, X¹⁰ is E. In some embodiments, X¹⁰ comprises a side chain comprising a polar group. In some embodiments, a cluster is enriched for amino acid residues whose side chains comprises polar groups at X¹⁰. In some embodiments, X¹⁰ is N. In some embodiments, X¹⁰ is T.

Various amino acid residues may be utilized for X¹². For example, in some embodiments, X¹² is Y. In some embodiments, X¹² is W. In some embodiments, X¹² is S.

Various amino acid residues may be utilized for X¹³. For example, in some embodiments, X¹³ is E. In some embodiments, X¹³ is D.

Various amino acid residues may be utilized for X¹⁴. For example, in some embodiments, X¹⁴ is I. In some embodiments, X¹⁴ is E. In some embodiments, X¹⁴ is W.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of SEQ ID NO: 1 or amino acid residues corresponding thereto: H219, H223, S250, F253, Y254, T257, N261, H260, L264, K292, A295, I296, N299, Y333, K335, W338, and T339. In some embodiments, it interacts with H219 of SEQ ID NO: 1 or an amino acid residue corresponding thereto. In some embodiments, it interacts with H223 of SEQ ID NO: 1 or an amino acid residue corresponding thereto. In some embodiments, it interacts with S250 of SEQ ID NO: 1 or an amino acid residue corresponding thereto. In some embodiments, it interacts with F253 of SEQ ID NO: 1 or an amino acid residue corresponding thereto. In some embodiments, it interacts with Y254 of SEQ ID NO: 1 or an amino acid residue corresponding thereto. In some embodiments, it interacts with T257 of SEQ ID NO: 1 or an amino acid residue corresponding thereto. In some embodiments, it interacts with N261 of SEQ ID NO: 1 or an amino acid residue corresponding thereto. In some embodiments, it interacts with H260 of SEQ ID NO: 1 or an amino acid residue corresponding thereto. In some embodiments, it interacts with L264 of SEQ ID NO: 1 or an amino acid residue corresponding thereto. In some embodiments, it interacts with K292 of SEQ ID NO: 1 or an amino acid residue corresponding thereto. In some embodiments, it interacts with A295 of SEQ ID NO: 1 or an amino acid residue corresponding thereto. In some embodiments, it interacts with I296 of SEQ ID NO: 1 or an amino acid residue corresponding thereto. In some embodiments, it interacts with N299 of SEQ ID NO: 1 or an amino acid residue corresponding thereto. In some embodiments, it interacts with Y333 of SEQ ID NO: 1 or an amino acid residue corresponding thereto. In some embodiments, it interacts with K335 of SEQ ID NO: 1 or an amino acid residue corresponding thereto. In some embodiments, it interacts with W338 of SEQ ID NO: 1 or an amino acid residue corresponding thereto. In some embodiments, it interacts with T339 of SEQ ID NO: 1 or an amino acid residue corresponding thereto.

In some embodiments, an agent comprising a cluster 32 sequence competes with axin for interaction with beta-catenin.

### Cluster C33

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C33 as described herein. In some embodiments, an agent comprises X³³X²X³X⁴X⁵X⁶X⁷X⁸X⁹X¹⁰X¹¹X¹²X¹³X¹⁴, wherein each of X³³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴X⁵X⁶X⁷X⁸X⁹X¹⁰X¹¹X¹²X¹³X¹⁴,

wherein:
each of X⁴, X⁵, X⁶, X⁷, X⁸, X⁹, X¹⁰, X¹¹, X¹², X¹³, and X¹⁴ is independently an amino acid residue;
X⁵ comprises a side chain comprising an acidic group;
X⁶ comprises a side chain comprising an aromatic group;
X¹⁰ comprises a side chain comprising an aromatic group;
X¹³comprises a hydrophobic side chain; and
X¹⁴ comprises a hydrophobic side chain.

Various amino acid residues may be utilized for X³³. For example, in some embodiments, X³³ is T. In some embodiments, X³³ is S. In some embodiments, X³³ is absent.

Various amino acid residues may be utilized for X². For example, in some embodiments, X² is H. In some embodiments, X² is E. In some embodiments, X² is absent.

Various amino acid residues may be utilized for X³. For example, in some embodiments, X³ is R. In some embodiments, X³ is L. In some embodiments, X³ is absent.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

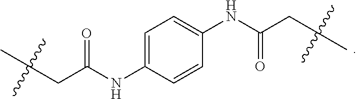 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprising acidic groups at X⁵. In some embodiments, X⁵ comprises a side chain comprising —COOH. In some embodiments, X⁵ is E. In some embodiments, X⁵ is D. In some embodiments, X⁵ comprises a side chain comprising a polar group. In some embodiments, a cluster is enriched for amino acid residues comprising a polar group at X⁵. In some embodiments, X⁵ is Q. In some embodiments, X⁵ is N.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ comprises an aromatic group. In some embodiments, the side chain of X⁶ is C₁₋₆ aliphatic. In some embodiments, the side chain of X⁶ is C₁₋₆ alkyl. In some embodiments, X⁶ is W. In some embodiments, X⁶ is M.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising an acidic group. In some embodiments, a cluster is enriched for amino acid residues whose side chains comprise acidic groups at X⁹. In some embodiments, X⁹ comprises a side chain comprising —COOH. In some embodiments, X⁹ is E. In some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, the side chain of X⁹ comprises an aromatic group. In some embodiments, X⁹ is I. In some embodiments, X⁹ is L. In some embodiments, X⁹ is V. In some embodiments, X⁹ is F. In some embodiments, X⁹ is M.

Various amino acid residues may be utilized for X¹⁰. For example, in some embodiments, X¹⁰ comprises a side chain comprising a basic group. In some embodiments, a cluster is enriched for amino acid residues whose side chains comprise basic groups at X¹⁰. In some embodiments, X¹⁰ comprises a side chain comprising an aromatic group. In some embodiments, a cluster is enriched for amino acid residues whose side chains comprise aromatic groups at X¹⁰. In some embodiments, X¹⁰ is H. In some embodiments, X¹⁰ is Y.

Various amino acid residues may be utilized for X¹². For example, in some embodiments, X¹² comprises a side chain comprising an acidic group. In some embodiments, a cluster is enriched for amino acid residues whose side chains comprising acidic groups at X¹². In some embodiments, X¹² comprises a side chain comprising —COOH. In some embodiments, X¹² is E. In some embodiments, X¹² is D. In some embodiments, X¹² comprises a side chain comprising a polar group. In some embodiments, a cluster is enriched for amino acid residues comprising a polar group at X¹². In some embodiments, X¹² is Q. In some embodiments, X¹² is S.

Various amino acid residues may be utilized for X¹³. For example, in some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, X¹³ is L.

Various amino acid residues may be utilized for X¹⁴. For example, in some embodiments, X¹⁴ comprises a hydrophobic side chain. In some embodiments, a cluster is enriched for amino acid residues comprising a hydrophobic side chain at X¹⁴. In some embodiments, the side chain of X¹⁴ is C₁₋₆ aliphatic. In some embodiments, the side chain of X¹⁴ is C₁₋₆ alkyl. In some embodiments, X¹⁴ is I. In some embodiments, X¹⁴ is L. In some embodiments, X¹⁴ is V.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of SEQ ID NO: 1 or amino acid residues corresponding thereto: H219, H223, S250, F253, Y254, T257, N261, H260, L264, K292, A295, I296, N299, Y333, K335, W338, and T339. In some embodiments, it interacts with H219 of SEQ ID NO: 1 or an amino acid residue corresponding thereto. In some embodiments, it interacts with H223 of SEQ ID NO: 1 or an amino acid residue corresponding thereto. In some embodiments, it interacts with S250 of SEQ ID NO: 1 or an amino acid residue corresponding thereto. In some embodiments, it interacts with F253 of SEQ ID NO: 1 or an amino acid residue corresponding thereto. In some embodiments, it interacts with Y254 of SEQ ID NO: 1 or an amino acid residue corresponding thereto. In some embodiments, it interacts with T257 of SEQ ID NO: 1 or an amino acid residue corresponding thereto. In some embodiments, it interacts with N261 of SEQ ID NO: 1 or an amino acid residue corresponding thereto. In some embodiments, it interacts with H260 of SEQ ID NO: 1 or an amino acid residue corresponding thereto. In some embodiments, it interacts with L264 of SEQ ID NO: 1 or an amino acid residue corresponding thereto. In some embodiments, it interacts with K292 of SEQ ID NO: 1 or an amino acid residue corresponding thereto. In some embodiments, it interacts with A295 of SEQ ID NO: 1 or an amino acid residue corresponding thereto. In some embodiments, it interacts with I296 of SEQ ID NO: 1 or an amino acid residue corresponding thereto. In some embodiments, it interacts with N299 of SEQ ID NO: 1 or an amino acid residue corresponding thereto. In some embodiments, it interacts with Y333 of SEQ ID NO: 1 or an amino acid residue corresponding thereto. In some embodiments, it interacts with K335 of SEQ ID NO: 1 or an amino acid residue corresponding thereto. In some embodiments, it interacts with W338 of SEQ ID NO: 1 or an amino acid residue corresponding thereto. In some embodiments, it interacts with T339 of SEQ ID NO: 1 or an amino acid residue corresponding thereto.

In some embodiments, an agent comprising a cluster 33 sequence competes with axin for interaction with beta-catenin.

### Cluster C34

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C34 as described herein. In some embodiments, an agent comprises X³⁴X²X³X⁴X⁵X⁶X⁷X⁸X⁹X¹⁰X¹¹X¹²X¹³X¹⁴, wherein each of X³⁴, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³⁴X²X³X⁴X⁵X⁶X⁷X⁸X⁹X¹⁰X¹¹X¹²X¹³X¹⁴,

wherein:
each of X³⁴, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group;
X⁵ comprises a side chain comprising an acidic side chain;
X⁶ comprises a side chain comprising a basic group or an aromatic group;
X⁹ comprises a hydrophobic side chain; and
X¹⁰ comprises a hydrophobic side chain.

Various amino acid residues may be utilized for X³⁴. For example, in some embodiments, X³⁴ is D. In some embodiments, X³⁴ is L. In some embodiments, X³⁴ is absent.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² comprises an aromatic group. In some embodiments, a cluster is enriched for amino acid residues comprising an aromatic group at X². In some embodiments, X² is W.

Various amino acid residues may be utilized for X³. For example, in some embodiments, X³ is E. In some embodiments, X³ is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

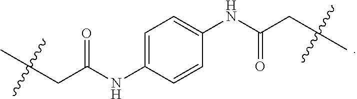 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprising acidic groups at X⁵. In some embodiments, X⁵ comprises a side chain comprising —COOH. In some embodiments, X⁵ is E. In some embodiments, X⁵ is D.

Various amino acid residues may be utilized for X⁶. For example, in some embodiments, X⁶ comprises a side chain comprising a basic group. In some embodiments, a cluster is enriched for amino acid residues whose side chains comprise basic groups at X⁶. In some embodiments, X⁶ comprises a side chain comprising an aromatic group. In some embodiments, a cluster is enriched for amino acid residues whose side chains comprise aromatic groups at X⁶. In some embodiments, X⁶ is H.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, X⁹ is L.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C₁₋₆ aliphatic. In some embodiments, the side chain of X¹⁰ is C₁₋₆ alkyl. In some embodiments, X¹⁰ is L. In some embodiments, X¹⁰ is I. In some embodiments, X¹⁰ is V. In some embodiments, X¹⁰ comprises a side chain comprising a polar group. In some embodiments, a cluster is enriched for amino acid residues comprising a polar group at X¹⁰. In some embodiments, X¹⁰ is N. In some embodiments, X¹⁰ is T.

Various amino acid residues may be utilized for X¹². For example, in some embodiments, X¹² is H. In some embodiments, X¹² is D. In some embodiments, X¹² is absent.

Various amino acid residues may be utilized for X¹³. For example, in some embodiments, X¹³ is Y. In some embodiments, X¹³ is V. In some embodiments, X¹³ is absent.

Various amino acid residues may be utilized for X¹⁴. For example, in some embodiments, X¹⁴ is W. In some embodiments, X¹⁴ is H. In some embodiments, X¹⁴ is absent.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of SEQ ID NO: 1 or amino acid residues corresponding thereto: H219, H223, S250, F253, Y254, T257, N261, H260, L264, K292, A295, I296, N299, Y333, K335, W338, and T339. In some embodiments, it interacts with H219 of SEQ ID NO: 1 or an amino acid residue corresponding thereto. In some embodiments, it interacts with H223 of SEQ ID NO: 1 or an amino acid residue corresponding thereto. In some embodiments, it interacts with S250 of SEQ ID NO: 1 or an amino acid residue corresponding thereto. In some embodiments, it interacts with F253 of SEQ ID NO: 1 or an amino acid residue corresponding thereto. In some embodiments, it interacts with Y254 of SEQ ID NO: 1 or an amino acid residue corresponding thereto. In some embodiments, it interacts with T257 of SEQ ID NO: 1 or an amino acid residue corresponding thereto. In some embodiments, it interacts with N261 of SEQ ID NO: 1 or an amino acid residue corresponding thereto. In some embodiments, it interacts with H260 of SEQ ID NO: 1 or an amino acid residue corresponding thereto. In some embodiments, it interacts with L264 of SEQ ID NO: 1 or an amino acid residue corresponding thereto. In some embodiments, it interacts with K292 of SEQ ID NO: 1 or an amino acid residue corresponding thereto. In some embodiments, it interacts with A295 of SEQ ID NO: 1 or an amino acid residue corresponding thereto. In some embodiments, it interacts with I296 of SEQ ID NO: 1 or an amino acid residue corresponding thereto. In some embodiments, it interacts with N299 of SEQ ID NO: 1 or an amino acid residue corresponding thereto. In some embodiments, it interacts with Y333 of SEQ ID NO: 1 or an amino acid residue corresponding thereto. In some embodiments, it interacts with K335 of SEQ ID NO: 1 or an amino acid residue corresponding thereto. In some embodiments, it interacts with W338 of SEQ ID NO: 1 or an amino acid residue corresponding thereto. In some embodiments, it interacts with T339 of SEQ ID NO: 1 or an amino acid residue corresponding thereto.

In some embodiments, an agent comprising a cluster 34 sequence competes with axin for interaction with beta-catenin.

### Cluster C35

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C35 as described herein. In some embodiments, an agent comprises X³⁵X²X³X⁴X⁵X⁶X⁷X⁸X⁹X¹⁰X¹¹X¹²X¹³X¹⁴, wherein each of X³⁵, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³⁵X²X³X⁴X⁵X⁶X⁷X⁸X⁹X¹⁰X¹¹X¹²X¹³X¹⁴,

wherein:
each of X³⁵, X², X³, X⁴, X⁵, X⁶, X⁷, X⁸, X⁹, X¹⁰, X¹¹, X¹², X¹³, and X¹⁴ is independently an amino acid residue;
X⁵ comprises a side chain comprising an aromatic group;
X⁶ comprises a side chain comprising an acidic group;
X⁹ comprises a side chain comprising an aromatic group; and
X¹³ comprises a side chain comprising an aromatic group.

Various amino acid residues may be utilized for X³⁵. For example, in some embodiments, X³⁵ comprises a hydrophobic side chain. In some embodiments, a cluster is enriched for amino acid residues comprising a hydrophobic side chain at X³⁵. In some embodiments, the side chain of X³⁵ is C₁₋₆ aliphatic. In some embodiments, the side chain of X³⁵ is C₁₋₆ alkyl. In some embodiments, X³⁵ is I. In some embodiments, X³⁵ is V. In some embodiments, X³⁵ is M

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² is C₁₋₆ aliphatic. In some embodiments, the side chain of X² is C₁₋₆ alkyl. In some embodiments, X² is I. In some embodiments, X² is L. In some embodiments, X² is M. In some embodiments, X² is V.

Various amino acid residues may be utilized for X³. For example, in some embodiments, X³ is H. In some embodiments, X³ is E.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

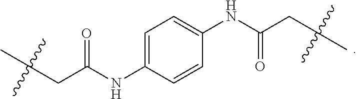 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, the side chain of X⁵ comprises an aromatic group. In some embodiments, a cluster is enriched for amino acid residues comprising an aromatic group at X⁵. In some embodiments, the side chain of X⁵ comprises a polar group. In some embodiments, a cluster is enriched for amino acid residues comprising a polar group at X⁵. In some embodiments, X⁵ comprises a side chain comprising —OH. In some embodiments, X⁵ is Y.

Various amino acid residues may be utilized for X⁶. For example, in some embodiments, X⁶ comprises a side chain comprising an acidic group. In some embodiments, a cluster is enriched for amino acid residues whose side chains comprise acidic groups at X⁶. In some embodiments, X⁶ comprises a side chain comprising —COOH. In some embodiments, X⁶ is E. In some embodiments, X⁶ is D. In some embodiments, X⁶ comprises a side chain comprising a polar group. In some embodiments, a cluster is enriched for amino acid residues comprising a polar group at X⁶. In some embodiments, X⁶ is Q.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ comprises an aromatic group. In some embodiments, a cluster is enriched for amino acid residues comprising an aromatic group at X⁹. In some embodiments, X⁹ is F. In some embodiments, X⁹ is Y.

Various amino acid residues may be utilized for X¹⁰. For example, in some embodiments, X¹⁰ is F. In some embodiments, X¹⁰ is I.

Various amino acid residues may be utilized for X¹². For example, in some embodiments, X¹² comprises a side chain comprising a polar group. In some embodiments, a cluster is enriched for amino acid residues whose side chains comprise polar groups at X¹². In some embodiments, X¹² is Q. In some embodiments, X¹² comprises a side chain comprising an acidic group. In some embodiments, a cluster is enriched for amino acid residues comprising an acidic group at X¹². In some embodiments, X¹² comprises a side chain comprising —COOH. In some embodiments, X¹² is D. In some embodiments, X¹² is E. In some embodiments, X¹² comprises a side chain comprising a basic group. In some embodiments, a cluster is enriched for amino acid residues comprising a basic group at X¹². In some embodiments, X¹² is H.

Various amino acid residues may be utilized for X¹³. For example, in some embodiments, the side chain of X¹³ comprises an aromatic group. In some embodiments, a cluster is enriched for amino acid residues comprising an aromatic group at X¹³. In some embodiments, the side chain of X¹³ comprises a polar group. In some embodiments, a cluster is enriched for amino acid residues comprising a polar group at X¹³. In some embodiments, X¹³ comprises a side chain comprising —OH. In some embodiments, X¹³ is Y. In some embodiments, X¹³ is N.

Various amino acid residues may be utilized for X¹⁴. For example, in some embodiments, X¹⁴ comprises a hydrophobic side chain. In some embodiments, a cluster is enriched for amino acid residues comprising a hydrophobic side chain at X¹⁴. In some embodiments, the side chain of X¹⁴ is C₁₋₆ aliphatic. In some embodiments, the side chain of X¹⁴ is C₁₋₆ alkyl. In some embodiments, the side chain of X¹⁴ comprises an aromatic group. In some embodiments, X¹⁴ is F. In some embodiments, X¹⁴ is M. In some embodiments, X¹⁴ is I. In some embodiments, X¹⁴ is V. In some embodiments, X¹⁴ is Y. In some embodiments, X¹⁴ is Q.

In some embodiments, an agent described herein, e.g., a stapled peptide, interacts with one or more (e.g., 1, 2, 3, 4, 5, 6, 7, 8, 9, 10, 11, 12, 13, or 14) or all of the following amino acid residues of SEQ ID NO: 1 or amino acid residues corresponding thereto: L519, I579, H578, R582, E620, C619, R661, F660, A657, T653, Y654, R612, G575, and C613. In some embodiments, it interacts with L519 of SEQ ID NO: 1 or an amino acid residue corresponding thereto. In some embodiments, it interacts with I579 of SEQ ID NO: 1 or an amino acid residue corresponding thereto. In some embodiments, it interacts with H578 of SEQ ID NO: 1 or an amino acid residue corresponding thereto. In some embodiments, it interacts with R582 of SEQ ID NO: 1 or an amino acid residue corresponding thereto. In some embodiments, it interacts with E620 of SEQ ID NO: 1 or an amino acid residue corresponding thereto. In some embodiments, it interacts with C619 of SEQ ID NO: 1 or an amino acid residue corresponding thereto. In some embodiments, it interacts with R661 of SEQ ID NO: 1 or an amino acid residue corresponding thereto. In some embodiments, it interacts with F660 of SEQ ID NO: 1 or an amino acid residue corresponding thereto. In some embodiments, it interacts with A657 of SEQ ID NO: 1 or an amino acid residue corresponding thereto. In some embodiments, it interacts with T653 of SEQ ID NO: 1 or an amino acid residue corresponding thereto. In some embodiments, it interacts with Y654 of SEQ ID NO: 1 or an amino acid residue corresponding thereto. In some embodiments, it interacts with R612 of SEQ ID NO: 1 or an amino acid residue corresponding thereto. In some embodiments, it interacts with G575 of SEQ ID NO: 1 or an amino acid residue corresponding thereto. In some embodiments, it interacts with C613 of SEQ ID NO: 1 or an amino acid residue corresponding thereto.

In some embodiments, an agent comprising a cluster 35 sequence does not compete with axin for interaction with beta-catenin.

### RNF31

In some embodiments, the present disclosure provides agents that can bind to and/or modulate activities of RNF31. In some embodiments, stapled peptides comprising sequences of certain clusters, e.g., cluster C41, cluster C42, cluster C43, cluster C44, and cluster C45, compete with Otulin for RNF31 interaction. In some embodiments, stapled peptides comprising sequences of certain clusters, e.g., cluster 46, cluster C47, and cluster C48, compete with Sharpin/SIPL1 for RNF31 interaction. In some embodiments, stapled peptides comprising sequences of certain clusters, e.g., cluster C41, cluster C42, cluster C43, cluster C44, and cluster C45, bind to RNF31 PUB domain. In some embodiments, stapled peptides comprising sequences of certain clusters, e.g., cluster 46, cluster C47, and cluster C48 bind to RNF31 UBA domain. In some embodiments, the present disclosure provides methods for reducing interactions with relevant partners, or reducing interactions at PUB or UBA domain, comprising contacting RNF31 with stapled peptides that compete with the partner or interact with RNF at PUB or UBA domain, or comprising administering or delivering to a system comprising RNF31 stapled peptides that compete with the partner or interact with RNF at PUB or UBA domain.

### Cluster C41

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C41 as described herein. In some embodiments, an agent comprises X⁴¹X²X³X⁴X⁵X⁶X⁷X⁸X⁹X¹⁰X¹¹X¹²X¹³X¹⁴, wherein each of X⁴¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴¹X²X³X⁴X⁵X⁶X⁷X⁸X⁹X¹⁰X¹¹X¹²X¹³X¹⁴,

wherein:
each of X⁴¹, X², X³, X⁴, X⁵, X⁶, X⁷, X⁸, X⁹, X¹⁰, X¹¹, X¹², X¹³, and X¹⁴ is independently an amino acid residue;
X⁶ comprises a side chain comprising an aromatic group;
X⁹ comprises a hydrophobic side chain;
X¹⁰ comprises a side chain comprising an aromatic group; and
X¹² comprises a side chain comprising an acidic group.

Various amino acid residues may be utilized for X⁴¹. Various amino acid residues may be utilized for X⁴¹. For example, in some embodiments, X⁴¹ comprises a hydrophobic side chain. In some embodiments, a cluster is enriched for amino acid residues comprising a hydrophobic side chain at X⁴¹. In some embodiments, the side chain of X⁴¹ comprises an aromatic group. In some embodiments, X⁴¹ is I. In some embodiments, X⁴¹ is Y. In some embodiments, X⁴¹ is W. In some embodiments, X⁴¹ is L. In some embodiments, X⁴¹ is A.

Various amino acid residues may be utilized for X². For example, in some embodiments, X² comprises a side chain comprising an acidic group. In some embodiments, a cluster is enriched for amino acid residues whose side chains comprising acidic groups at X². In some embodiments, X² comprises a side chain comprising —COOH. In some embodiments, X² is E. In some embodiments, X² is D. In some embodiments, X² comprises a side chain comprising a polar group. In some embodiments, X² is T. In some embodiments, X² is V.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, X³ comprises a side chain comprising a polar group. In some embodiments, X³ is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

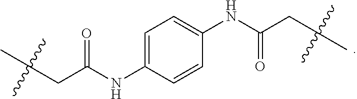 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprising acidic groups at X⁵. In some embodiments, X⁵ comprises a side chain comprising —COOH. In some embodiments, X⁵ is D. In some embodiments, X⁵ is E. In some embodiments, X³ comprises a side chain comprising a polar group. In some embodiments, X⁵ is S. In some embodiments, X⁵ is A.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ comprises an aromatic group. In some embodiments, a cluster is enriched for amino acid residues comprising an aromatic group at X⁶. In some embodiments, X⁶ is W. In some embodiments, X⁶ is D.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, X⁹ is L. In some embodiments, X⁹ is I. In some embodiments, X⁹ is M.

Various amino acid residues may be utilized for X¹⁰. For example, in some embodiments, the side chain of X¹⁰ comprises an aromatic group. In some embodiments, a cluster is enriched for amino acid residues comprising an aromatic group at X¹⁰. In some embodiments, X¹⁰ comprises a side chain comprising —OH. In some embodiments, X¹⁰ is Y.

Various amino acid residues may be utilized for X¹². For example, in some embodiments, X¹² comprises a side chain comprising an acidic group. In some embodiments, a cluster is enriched for amino acid residues whose side chains comprising acidic groups at X¹². In some embodiments, X¹² comprises a side chain comprising —COOH. In some embodiments, X¹² is D. In some embodiments, X¹² is E. In some embodiments, X¹² comprises a side chain comprising a polar group. In some embodiments, X¹² is N. In some embodiments, X¹² is S.

Various amino acid residues may be utilized for X¹³. For example, in some embodiments, X¹³ comprises a side chain comprising an acidic group. In some embodiments, a cluster is enriched for amino acid residues whose side chains comprising acidic groups at X¹³. In some embodiments, X¹³ comprises a side chain comprising —COOH. In some embodiments, X¹³ is D. In some embodiments, X¹³ is E. In some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, X¹³ is M.

Various amino acid residues may be utilized for X¹⁴. For example, in some embodiments, X¹⁴ is Q.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of SEQ ID NO: 2 or amino acid residues corresponding thereto: Thr74, Asn77, Ile78, Lys81, Tyr82, Asn85, Leu86, Pro92, Tyr94, Trp95, Val98, Asn102, Val104, Thr108, and Tyr124. In some embodiments, it interacts with Thr74 of SEQ ID NO: 2 or an amino acid residue corresponding thereto. In some embodiments, it interacts with Asn77 of SEQ ID NO: 2 or an amino acid residue corresponding thereto. In some embodiments, it interacts with Ile78 of SEQ ID NO: 2 or an amino acid residue corresponding thereto. In some embodiments, it interacts with Lys81 of SEQ ID NO: 2 or an amino acid residue corresponding thereto. In some embodiments, it interacts with Tyr82 of SEQ ID NO: 2 or an amino acid residue corresponding thereto. In some embodiments, it interacts with Asn85 of SEQ ID NO: 2 or an amino acid residue corresponding thereto. In some embodiments, it interacts with Leu86 of SEQ ID NO: 2 or an amino acid residue corresponding thereto. In some embodiments, it interacts with Pro92 of SEQ ID NO: 2 or an amino acid residue corresponding thereto. In some embodiments, it interacts with Tyr94 of SEQ ID NO: 2 or an amino acid residue corresponding thereto. In some embodiments, it interacts with Trp95 of SEQ ID NO: 2 or an amino acid residue corresponding thereto. In some embodiments, it interacts with Val98 of SEQ ID NO: 2 or an amino acid residue corresponding thereto. In some embodiments, it interacts with Asn102 of SEQ ID NO: 2 or an amino acid residue corresponding thereto. In some embodiments, it interacts with Val104 of SEQ ID NO: 2 or an amino acid residue corresponding thereto. In some embodiments, it interacts with Thr108 of SEQ ID NO: 2 or an amino acid residue corresponding thereto. In some embodiments, it interacts with Tyr124 of SEQ ID NO: 2 or an amino acid residue corresponding thereto.

(SEQ ID NO: 2)
MPGEEEERAFLVAREELASALRRDSGQAFSLEQLRPLLASSLPLAARYL
QLDAARLVRCNAHGEPRNYLNTLSTALNILEKYGRNLLSPQRPRYWRGV
KFNNPVFRSTVDAVQGGRDVLRLYGYTEEQPDGLSFPEGQEEPDEHQVA
TVTLEVLLLRTELSLLLQNTHPRQQALEQLLEDKVEDDMLQLSEFDPLL
REIAPGPLTTPSVPGSTPGPCFLCGSAPGTLHCPSCKQALCPACDHLFH
GHPSRAHHLRQTLPGVLQGTHLSPSLPASAQPRPQSTSLLALGDSSLSS
PNPASAHLPWHCAACAMLNEPWAVLCVACDRPRGCKGLGLGTEGPQGTG
GLEPDLARGRWACQSCTFENEAAAVLCSICERPRLAQPPSLVVDSRDAG
ICLQPLQQGDALLASAQSQVWYCIHCTFCNSSPGWVCVMCNRTSSPIPA
QHAPRPYASSLEKGPPKPGPPRRLSAPLPSSCGDPEKQRQDKMREEGLQ
LVSMIREGEAAGACPEEIFSALQYSGTEVPLQWLRSELPYVLEMVAELA
GQQDPGLGAFSCQEARRAWLDRHGNLDEAVEECVRTRRRKVQELQSLGF
GPEEGSLQALFQHGGDVSRALTELQRQRLEPFRQRLWDSGPEPTPSWDG
PDKQSLVRRLLAVYALPSWGRAELALSLLQETPRNYELGDVVEAVRHSQ
DRAFLRRLLAQECAVCGWALPHNRMQALTSCECTICPDCFRQHFTIALK
EKHITDMVCPACGRPDLTDDTQLLSYFSTLDIQLRESLEPDAYALFHKK
LTEGVLMRDPKFLWCAQCSFGFIYEREQLEATCPQCHQTFCVRCKRQWE
EQHRGRSCEDFQNWKRMNDPEYQAQGLAMYLQENGIDCPKCKFSYALAR
GGCMHFHCTQCRHQFCSGCYNAFYAKNKCPEPNCRVKKSLHGHHPRDCL
FYLRDWTALRLQKLLQDNNVMENTEPPAGARAVPGGGCRVIEQKEVPNG
LRDEACGKETPAGYAGLCQAHYKEYLVSLINAHSLDPATLYEVEELETA
TERYLHVRPQPLAGEDPPAYQARLLQKLTEEVPLGQSIPRRRK

In some embodiments, an agent comprising a cluster 41 sequence interacts with RNF31 PUB domain.

### Cluster C42

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C42 as described herein. In some embodiments, an agent comprises X⁴²X²X³X⁴X⁵X⁶X⁷X⁸X⁹X¹⁰X¹¹X¹²X¹³X¹⁴, wherein each of X⁴², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴²X²X³X⁴X⁵X⁶X⁷X⁸X⁹X¹⁰X¹¹X¹²X¹³X¹⁴,

wherein:
each of X⁴², X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group;
X⁵ comprises a hydrophobic side chain; and
X⁶ comprises a side chain comprising an aromatic group.

Various amino acid residues may be utilized for X⁴². For example, in some embodiments, X⁴² comprises a side chain comprising an aromatic group. In some embodiments, a cluster is enriched for amino acid residues whose side chains comprise aromatic groups at X⁴². In some embodiments, X⁴² is W. In some embodiments, X⁴² is Y. In some embodiments, X⁴² is F.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² is C₁₋₆ aliphatic. In some embodiments, the side chain of X² is C₁₋₆ alkyl. In some embodiments, the side chain of X² comprises an aromatic group. In some embodiments, X² is W. In some embodiments, X² is M. In some embodiments, X² is H.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, X³ comprises a side chain comprising a polar group. In some embodiments, X³ is N.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

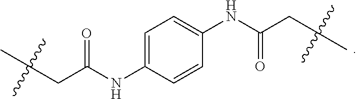 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is L. In some embodiments, X⁵ is I. In some embodiments, X⁵ is M.

Various amino acid residues may be utilized for X⁶. For example, in some embodiments, the side chain of X⁶ comprises an aromatic group. In some embodiments, a cluster is enriched for amino acid residues comprising an aromatic group at X⁶. In some embodiments, X⁶ comprises a side chain comprising —OH. In some embodiments, X⁶ is Y.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ is W.

Various amino acid residues may be utilized for X¹⁰. For example, in some embodiments, X¹⁰ comprises a side chain comprising an acidic group. In some embodiments, a cluster is enriched for amino acid residues whose side chains comprising acidic groups at X¹⁰. In some embodiments, X¹⁰ comprises a side chain comprising —COOH. In some embodiments, X¹⁰ is D. In some embodiments, X¹⁰ is E. In some embodiments, X¹⁰ comprises a side chain comprising a polar group. In some embodiments, X¹⁰ is T. In some embodiments, X¹⁰ is S.

Various amino acid residues may be utilized for X¹². In some embodiments, X¹² is D. In some embodiments, X¹² is Y.

Various amino acid residues may be utilized for X¹³.

Various amino acid residues may be utilized for X¹⁴.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of SEQ ID NO: 2 or amino acid residues corresponding thereto: Thr74, Asn77, Ile78, Lys81, Tyr82, Asn85, Leu86, Pro92, Tyr94, Trp95, Val98, Asn102, Val104, Thr108, and Tyr124. In some embodiments, it interacts with Thr74 of SEQ ID NO: 2 or an amino acid residue corresponding thereto. In some embodiments, it interacts with Asn77 of SEQ ID NO: 2 or an amino acid residue corresponding thereto. In some embodiments, it interacts with Ile78 of SEQ ID NO: 2 or an amino acid residue corresponding thereto. In some embodiments, it interacts with Lys81 of SEQ ID NO: 2 or an amino acid residue corresponding thereto. In some embodiments, it interacts with Tyr82 of SEQ ID NO: 2 or an amino acid residue corresponding thereto. In some embodiments, it interacts with Asn85 of SEQ ID NO: 2 or an amino acid residue corresponding thereto. In some embodiments, it interacts with Leu86 of SEQ ID NO: 2 or an amino acid residue corresponding thereto. In some embodiments, it interacts with Pro92 of SEQ ID NO: 2 or an amino acid residue corresponding thereto. In some embodiments, it interacts with Tyr94 of SEQ ID NO: 2 or an amino acid residue corresponding thereto. In some embodiments, it interacts with Trp95 of SEQ ID NO: 2 or an amino acid residue corresponding thereto. In some embodiments, it interacts with Val98 of SEQ ID NO: 2 or an amino acid residue corresponding thereto. In some embodiments, it interacts with Asn102 of SEQ ID NO: 2 or an amino acid residue corresponding thereto. In some embodiments, it interacts with Val104 of SEQ ID NO: 2 or an amino acid residue corresponding thereto. In some embodiments, it interacts with Thr108 of SEQ ID NO: 2 or an amino acid residue corresponding thereto. In some embodiments, it interacts with Tyr124 of SEQ ID NO: 2 or an amino acid residue corresponding thereto.

In some embodiments, an agent comprising a cluster 42 sequence interacts with RNF31 PUB domain.

### Cluster C43

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C43 as described herein. In some embodiments, an agent comprises X⁴³X²X³X⁴X⁵X⁶X⁷X⁸X⁹X¹⁰X¹¹X¹²X¹³X¹⁴, wherein each of X⁴³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴³X²X³X⁴X⁵X⁶X⁷X⁸X⁹X¹⁰X¹¹X¹²X¹³X¹⁴,

wherein:
each of X⁴³, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an acidic group;
X³ comprises a side chain comprising an aromatic group;
X⁵ comprises a hydrophobic side chain;
X⁶ comprises a side chain comprising a polar group or an aromatic group; and
X¹⁰ comprises a side chain comprising an acidic group.

Various amino acid residues may be utilized for X⁴³. For example, in some embodiments, the side chain of X⁴³ comprises an aromatic group. In some embodiments, a cluster is enriched for amino acid residues comprising an aromatic group at X⁴³. In some embodiments, X⁴³ is Y. In some embodiments, X⁴³ is W. In some embodiments, X⁴³ is H.

Various amino acid residues may be utilized for X². For example, in some embodiments, X² comprises a side chain comprising an acidic group. In some embodiments, a cluster is enriched for amino acid residues whose side chains comprising acidic groups at X². In some embodiments, X² comprises a side chain comprising —COOH. In some embodiments, X² is D. In some embodiments, X² is E. In some embodiments, X² is W.

Various amino acid residues may be utilized for X³. For example, in some embodiments, the side chain of X³ comprises an aromatic group. In some embodiments, a cluster is enriched for amino acid residues comprising an aromatic group at X³. In some embodiments, X⁴³ is W. In some embodiments, X³ is Y.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

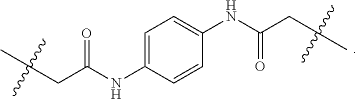 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is L. In some embodiments, X⁵ is M.

Various amino acid residues may be utilized for X⁶. For example, in some embodiments, the side chain of X⁶ comprises an aromatic group. In some embodiments, a cluster is enriched for amino acid residues comprising an aromatic group at X⁶. In some embodiments, X⁶ comprises a side chain comprising —OH. In some embodiments, X⁶ is Y.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ is D. In some embodiments, X⁹ is Y. In some embodiments, X⁹ is E.

Various amino acid residues may be utilized for X¹⁰. For example, in some embodiments, X¹⁰ comprises a side chain comprising an acidic group. In some embodiments, a cluster is enriched for amino acid residues whose side chains comprising acidic groups at X¹⁰. In some embodiments, X¹⁰ comprises a side chain comprising —COOH. In some embodiments, X¹⁰ is D. In some embodiments, X¹⁰ is E. In some embodiments, X¹⁰ is N.

Various amino acid residues may be utilized for X¹².

Various amino acid residues may be utilized for X¹³.

Various amino acid residues may be utilized for X¹⁴.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of SEQ ID NO: 2 or amino acid residues corresponding thereto: Thr74, Asn77, Ile78, Lys81, Tyr82, Asn85, Leu86, Pro92, Tyr94, Trp95, Val98, Asn102, Val104, Thr108, and Tyr124. In some embodiments, it interacts with Thr74 of SEQ ID NO: 2 or an amino acid residue corresponding thereto. In some embodiments, it interacts with Asn77 of SEQ ID NO: 2 or an amino acid residue corresponding thereto. In some embodiments, it interacts with Ile78 of SEQ ID NO: 2 or an amino acid residue corresponding thereto. In some embodiments, it interacts with Lys81 of SEQ ID NO: 2 or an amino acid residue corresponding thereto. In some embodiments, it interacts with Tyr82 of SEQ ID NO: 2 or an amino acid residue corresponding thereto. In some embodiments, it interacts with Asn85 of SEQ ID NO: 2 or an amino acid residue corresponding thereto. In some embodiments, it interacts with Leu86 of SEQ ID NO: 2 or an amino acid residue corresponding thereto. In some embodiments, it interacts with Pro92 of SEQ ID NO: 2 or an amino acid residue corresponding thereto. In some embodiments, it interacts with Tyr94 of SEQ ID NO: 2 or an amino acid residue corresponding thereto. In some embodiments, it interacts with Trp95 of SEQ ID NO: 2 or an amino acid residue corresponding thereto. In some embodiments, it interacts with Val98 of SEQ ID NO: 2 or an amino acid residue corresponding thereto. In some embodiments, it interacts with Asn102 of SEQ ID NO: 2 or an amino acid residue corresponding thereto. In some embodiments, it interacts with Val104 of SEQ ID NO: 2 or an amino acid residue corresponding thereto. In some embodiments, it interacts with Thr108 of SEQ ID NO: 2 or an amino acid residue corresponding thereto. In some embodiments, it interacts with Tyr124 of SEQ ID NO: 2 or an amino acid residue corresponding thereto.

In some embodiments, an agent comprising a cluster 43 sequence interacts with RNF31 PUB domain.

### Cluster C44

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C44 as described herein. In some embodiments, an agent comprises X⁴⁴X²X³X⁴X⁵X⁶X⁷X⁸X⁹X¹⁰X¹¹X¹²X¹³X¹⁴, wherein each of X⁴, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴⁴X²X³X⁴X⁵X⁶X⁷X⁸X⁹X¹⁰X¹¹X¹²X¹³X¹,

wherein:
each of X⁴⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain;
X¹³ comprises a side chain comprising a polar group; and
X¹⁴ comprises a side chain comprising s polar group or an aromatic group.

Various amino acid residues may be utilized for X⁴⁴. Various amino acid residues may be utilized for X⁴⁴. For example, in some embodiments, X⁴⁴ comprises a hydrophobic side chain. In some embodiments, a cluster is enriched for amino acid residues comprising a hydrophobic side chain at X⁴⁴. In some embodiments, the side chain of X⁴⁴ is C₁₋₆ aliphatic. In some embodiments, the side chain of X⁴⁴ is C₁₋₆ alkyl. In some embodiments, the side chain of X⁴⁴ comprises an aromatic group. In some embodiments, X⁴⁴ is I. In some embodiments, X⁴⁴ is F. In some embodiments, X⁴⁴ is A.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, X³ comprises a side chain comprising a polar group. In some embodiments, X³ is T. In some embodiments, X³ is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

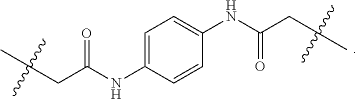 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ is Q.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, the side chain of X⁶ comprises an aromatic group. In some embodiments, X⁶ is L. In some embodiments, X⁶ is I. In some embodiments, X⁶ is V. In some embodiments, X⁶ is F.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, the side chain of X⁹ comprises an aromatic group. In some embodiments, X⁹ is A. In some embodiments, X⁹ is M. In some embodiments, X⁹ is L. In some embodiments, X⁹ is Q. In some embodiments, X⁹ is D.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C₁₋₆ aliphatic. In some embodiments, the side chain of X¹⁰ is C₁₋₆ alkyl. In some embodiments, X¹⁰ is V. In some embodiments, X¹⁰ is I. In some embodiments, X¹⁰ is T.

Various amino acid residues may be utilized for X¹². For example, in some embodiments, X¹² comprises a side chain comprising a polar group. In some embodiments, a cluster is enriched for amino acid residues comprising a polar group at X¹². In some embodiments, X¹² comprises a side chain comprising —OH. In some embodiments, X¹² is S. In some embodiments, X¹² is T. In some embodiments, X¹² is M. In some embodiments, X¹² is A.

Various amino acid residues may be utilized for X¹³. For example, in some embodiments, X¹³ comprises a side chain comprising a polar group. In some embodiments, a cluster is enriched for amino acid residues comprising a polar group at X¹³. In some embodiments, X¹³ comprises a side chain comprising —OH. In some embodiments, X¹³ is T.

Various amino acid residues may be utilized for X¹⁴. For example, in some embodiments, the side chain of X¹⁴ comprises an aromatic group. In some embodiments, a cluster is enriched for amino acid residues comprising an aromatic group at X¹⁴. In some embodiments, X¹⁴ comprises a side chain comprising —OH. In some embodiments, X¹⁴ is Y.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of SEQ ID NO: 2 or amino acid residues corresponding thereto: Thr74, Asn77, Ile78, Lys81, Tyr82, Asn85, Leu86, Pro92, Tyr94, Trp95, Val98, Asn102, Val104, Thr108, and Tyr124. In some embodiments, it interacts with Thr74 of SEQ ID NO: 2 or an amino acid residue corresponding thereto. In some embodiments, it interacts with Asn77 of SEQ ID NO: 2 or an amino acid residue corresponding thereto. In some embodiments, it interacts with Ile78 of SEQ ID NO: 2 or an amino acid residue corresponding thereto. In some embodiments, it interacts with Lys81 of SEQ ID NO: 2 or an amino acid residue corresponding thereto. In some embodiments, it interacts with Tyr82 of SEQ ID NO: 2 or an amino acid residue corresponding thereto. In some embodiments, it interacts with Asn85 of SEQ ID NO: 2 or an amino acid residue corresponding thereto. In some embodiments, it interacts with Leu86 of SEQ ID NO: 2 or an amino acid residue corresponding thereto. In some embodiments, it interacts with Pro92 of SEQ ID NO: 2 or an amino acid residue corresponding thereto. In some embodiments, it interacts with Tyr94 of SEQ ID NO: 2 or an amino acid residue corresponding thereto. In some embodiments, it interacts with Trp95 of SEQ ID NO: 2 or an amino acid residue corresponding thereto. In some embodiments, it interacts with Val98 of SEQ ID NO: 2 or an amino acid residue corresponding thereto. In some embodiments, it interacts with Asn102 of SEQ ID NO: 2 or an amino acid residue corresponding thereto. In some embodiments, it interacts with Val104 of SEQ ID NO: 2 or an amino acid residue corresponding thereto. In some embodiments, it interacts with Thr108 of SEQ ID NO: 2 or an amino acid residue corresponding thereto. In some embodiments, it interacts with Tyr124 of SEQ ID NO: 2 or an amino acid residue corresponding thereto.

In some embodiments, an agent comprising a cluster 44 sequence interacts with RNF31 PUB domain.

### Cluster C45

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C45 as described herein. In some embodiments, an agent comprises X⁴⁵X²X³X⁴X⁵X⁶X⁷X⁸X⁹X¹⁰X¹¹X¹²X¹³X¹⁴, wherein each of X⁴⁵, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴⁵X²X³X⁴X⁵X⁶X⁷X⁸X⁹X¹⁰X¹¹X¹²X¹³X¹⁴,

wherein:
each of X⁴⁵, X², X³, X⁴, X⁵, X⁶, X⁷, X⁸, X⁹, X¹⁰, X¹¹, X¹², X¹³, and X¹⁴ is independently an amino acid residue;
X⁶ comprises a side chain comprising an acidic group;
X⁹ comprises a hydrophobic side chain;
X¹² comprises a side chain comprising a polar group; and
X¹³ comprises a side chain comprising a polar group or an aromatic group.

Various amino acid residues may be utilized for X4⁵.

Various amino acid residues may be utilized for X². For example, in some embodiments, X² comprises a side chain comprising an acidic group. In some embodiments, a cluster is enriched for amino acid residues whose side chains comprising acidic groups at X². In some embodiments, X² comprises a side chain comprising —COOH. In some embodiments, X² is E. In some embodiments, X² is D.

Various amino acid residues may be utilized for X³.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

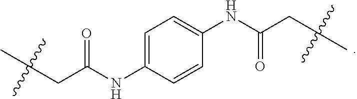 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, the side chain of X⁵ comprises an aromatic group. In some embodiments, X⁵ is W. In some embodiments, X⁵ is L. In some embodiments, X⁵ is I. In some embodiments, X⁵ is F.

Various amino acid residues may be utilized for X⁶. For example, in some embodiments, X⁶ comprises a side chain comprising an acidic group. In some embodiments, a cluster is enriched for amino acid residues whose side chains comprising acidic groups at X⁶. In some embodiments, X² comprises a side chain comprising —COOH. In some embodiments, X⁶ is D. In some embodiments, X⁶ is E. In some embodiments, the side chain of X⁶ comprises a polar group. In some embodiments, X⁶ is N. In some embodiments, X⁶ is S.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, X⁹ is V. In some embodiments, X⁹ is I. In some embodiments, X⁹ is M.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C₁₋₆ aliphatic. In some embodiments, the side chain of X¹⁰ is C₁₋₆ alkyl. In some embodiments, X¹⁰ is M. In some embodiments, X¹⁰ is L. In some embodiments, X¹⁰ is A.

Various amino acid residues may be utilized for X¹². For example, in some embodiments, X¹² comprises a side chain comprising a polar group. In some embodiments, a cluster is enriched for amino acid residues comprising a polar group at X¹². In some embodiments, X¹² comprises a side chain comprising —OH. In some embodiments, X¹² is T. In some embodiments, X¹² is S.

Various amino acid residues may be utilized for X¹³. For example, in some embodiments, the side chain of X¹³ comprises an aromatic group. In some embodiments, a cluster is enriched for amino acid residues comprising an aromatic group at X¹³. In some embodiments, X¹³ comprises a side chain comprising a polar group. In some embodiments, a cluster is enriched for amino acid residues comprising a polar group at X¹³. In some embodiments, X¹³ comprises a side chain comprising —OH. In some embodiments, X¹³ is Y.

Various amino acid residues may be utilized for X¹⁴. For example, in some embodiments, X¹⁴ comprises a side chain comprising a polar group. In some embodiments, a cluster is enriched for amino acid residues comprising a polar group at X¹⁴. In some embodiments, X¹⁴ comprises a side chain comprising —OH. In some embodiments, X¹⁴ is Y. In some embodiments, X¹⁴ is T. In some embodiments, X¹⁴ is S.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of SEQ ID NO: 2 or amino acid residues corresponding thereto: Thr74, Asn77, Ile78, Lys81, Tyr82, Asn85, Leu86, Pro92, Tyr94, Trp95, Val98, Asn102, Val104, Thr108, and Tyr124. In some embodiments, it interacts with Thr74 of SEQ ID NO: 2 or an amino acid residue corresponding thereto. In some embodiments, it interacts with Asn77 of SEQ ID NO: 2 or an amino acid residue corresponding thereto. In some embodiments, it interacts with Ile78 of SEQ ID NO: 2 or an amino acid residue corresponding thereto. In some embodiments, it interacts with Lys81 of SEQ ID NO: 2 or an amino acid residue corresponding thereto. In some embodiments, it interacts with Tyr82 of SEQ ID NO: 2 or an amino acid residue corresponding thereto. In some embodiments, it interacts with Asn85 of SEQ ID NO: 2 or an amino acid residue corresponding thereto. In some embodiments, it interacts with Leu86 of SEQ ID NO: 2 or an amino acid residue corresponding thereto. In some embodiments, it interacts with Pro92 of SEQ ID NO: 2 or an amino acid residue corresponding thereto. In some embodiments, it interacts with Tyr94 of SEQ ID NO: 2 or an amino acid residue corresponding thereto. In some embodiments, it interacts with Trp95 of SEQ ID NO: 2 or an amino acid residue corresponding thereto. In some embodiments, it interacts with Val98 of SEQ ID NO: 2 or an amino acid residue corresponding thereto. In some embodiments, it interacts with Asn102 of SEQ ID NO: 2 or an amino acid residue corresponding thereto. In some embodiments, it interacts with Val104 of SEQ ID NO: 2 or an amino acid residue corresponding thereto. In some embodiments, it interacts with Thr108 of SEQ ID NO: 2 or an amino acid residue corresponding thereto. In some embodiments, it interacts with Tyr124 of SEQ ID NO: 2 or an amino acid residue corresponding thereto.

In some embodiments, an agent comprising a cluster 45 sequence interacts with RNF31 PUB domain.

### Cluster C46

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C46 as described herein. In some embodiments, an agent comprises X⁴⁶X²X³X⁴X⁵X⁶X⁷X⁸X⁹X¹⁰X¹¹X¹²X¹³X¹⁴, wherein each of X⁴⁶, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴⁶X²X³X⁴X⁵X⁶X⁷X⁸X⁹X¹⁰X¹¹X¹²X¹³X¹⁴,

wherein:
each of X⁴⁶, X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain or a side chain comprising an aromatic group;
X⁶ comprises a side chain comprising a polar group;
X⁹ comprises a hydrophobic side chain
X¹⁰ comprises a side chain comprising an aromatic group;

Various amino acid residues may be utilized for X⁴⁶. For example, in some embodiments, X⁴⁶ comprises a hydrophobic side chain. In some embodiments, a cluster is enriched for amino acid residues comprising a hydrophobic side chain at X⁴⁶. In some embodiments, the side chain of X⁴⁶ is C₁₋₆ aliphatic. In some embodiments, the side chain of X⁴⁶ is C₁₋₆ alkyl. In some embodiments, X⁴⁶ is M. In some embodiments, X⁴⁶ is L. In some embodiments, X⁴⁶ is A. In some embodiments, X⁴⁶ is Q.

Various amino acid residues may be utilized for X². In some embodiments, X² is Q. In some embodiments, X² is R. In some embodiments, X² is M.

Various amino acid residues may be utilized for X³. In some embodiments, X² is R.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

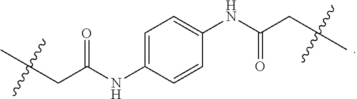 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, the side chain of X⁵ comprises an aromatic group. In some embodiments, X⁵ is L. In some embodiments, X⁵ is F. In some embodiments, X⁵ comprises a side chain comprising —OH. In some embodiments, X⁵ is Y. In some embodiments, X⁵ is T.

Various amino acid residues may be utilized for X⁶. For example, in some embodiments, X⁶ comprises a side chain comprising a polar group. In some embodiments, a cluster is enriched for amino acid residues comprising a polar group at X⁶. In some embodiments, X⁶ comprises a side chain comprising —OH. In some embodiments, X⁶ is S.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, the side chain of X⁹ comprises an aromatic group. In some embodiments, X⁹ is V. In some embodiments, X⁹ is I. In some embodiments, X⁹ is F. In some embodiments, X⁹ is M.

Various amino acid residues may be utilized for X¹⁰. For example, in some embodiments, the side chain of X¹⁰ comprises an aromatic group. In some embodiments, a cluster is enriched for amino acid residues comprising an aromatic group at X¹⁰. In some embodiments, X¹⁰ is Y. In some embodiments, X¹⁰ is W. In some embodiments, X¹⁰ is F.

Various amino acid residues may be utilized for X¹². In some embodiments, X¹² is Q. In some embodiments, X¹² is A. In some embodiments, X¹² is S. In some embodiments, X¹² is N.

Various amino acid residues may be utilized for X¹³. For example, in some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, the side chain of X¹³ comprises an aromatic group. In some embodiments, X¹³ is I. In some embodiments, X¹³ is M. In some embodiments, X¹³ is F.

Various amino acid residues may be utilized for X¹⁴. In some embodiments, X¹⁴ is S. In some embodiments, X¹⁴ is F. In some embodiments, X¹⁴ is Y. In some embodiments, X¹⁴ is H.

In some embodiments, an agent described herein, e.g., a stapled peptide, interacts with one or more (e.g., 1, 2, 3, 4, 5, 6, 7, 8, 9, 10, 11, 12, 13, 14, 15, or 16) or all of the following amino acid residues of SEQ ID NO: 2 or amino acid residues corresponding thereto: Glu506, Phe509, Ser510, Leu524, Arg525, Leu528, Tyr530, Val531, Met534, Leu538, Trp558, His562, Gly563, Gly563, Gly563, and Leu565. In some embodiments, it interacts with Glu506 of SEQ ID NO: 2 or an amino acid residue corresponding thereto. In some embodiments, it interacts with Phe509 of SEQ ID NO: 2 or an amino acid residue corresponding thereto. In some embodiments, it interacts with Ser510 of SEQ ID NO: 2 or an amino acid residue corresponding thereto. In some embodiments, it interacts with Leu524 of SEQ ID NO: 2 or an amino acid residue corresponding thereto. In some embodiments, it interacts with Arg525 of SEQ ID NO: 2 or an amino acid residue corresponding thereto. In some embodiments, it interacts with Leu528 of SEQ ID NO: 2 or an amino acid residue corresponding thereto. In some embodiments, it interacts with Tyr530 of SEQ ID NO: 2 or an amino acid residue corresponding thereto. In some embodiments, it interacts with Val531 of SEQ ID NO: 2 or an amino acid residue corresponding thereto. In some embodiments, it interacts with Met534 of SEQ ID NO: 2 or an amino acid residue corresponding thereto. In some embodiments, it interacts with Leu538 of SEQ ID NO: 2 or an amino acid residue corresponding thereto. In some embodiments, it interacts with Trp558 of SEQ ID NO: 2 or an amino acid residue corresponding thereto. In some embodiments, it interacts with His562 of SEQ ID NO: 2 or an amino acid residue corresponding thereto. In some embodiments, it interacts with Gly563 of SEQ ID NO: 2 or an amino acid residue corresponding thereto. In some embodiments, it interacts with Gly563 of SEQ ID NO: 2 or an amino acid residue corresponding thereto. In some embodiments, it interacts with Gly563 of SEQ ID NO: 2 or an amino acid residue corresponding thereto. In some embodiments, it interacts with Leu565 of SEQ ID NO: 2 or an amino acid residue corresponding thereto.

In some embodiments, an agent comprising a cluster 41 sequence interacts with RNF31 UBA domain.

### Cluster C47

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C47 as described herein. In some embodiments, an agent comprises X⁴⁷X²X³X⁴X⁵X⁶X⁷X⁸X⁹X¹⁰X¹¹X¹²X¹³X¹⁴, wherein each of X⁴⁷, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴⁷X²X³X⁴X⁵X⁶X⁷X⁸X⁹X¹⁰X¹¹X¹²X¹³X¹⁴,

wherein:
each of X⁴⁷, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising a polar group;
X⁵ comprises a hydrophobic side chain;
X⁶ comprises a side chain comprising a polar group or an aromatic group; and
X⁹ comprises a hydrophobic side chain.

Various amino acid residues may be utilized for X⁴⁷. For example, in some embodiments, X⁴⁷ comprises a hydrophobic side chain. In some embodiments, a cluster is enriched for amino acid residues comprising a hydrophobic side chain at X⁴⁷. In some embodiments, the side chain of X⁴⁷ comprises an aromatic group. In some embodiments, the side chain of X⁴⁷ is C₁₋₆ aliphatic. In some embodiments, the side chain of X⁴⁷ is C₁₋₆ alkyl. In some embodiments, X⁴⁷ is F. In some embodiments, X⁴⁷ is L.

Various amino acid residues may be utilized for X². For example, in some embodiments, the side chain of X² comprises a polar group. In some embodiments, a cluster is enriched for amino acid residues comprising a polar group at X². In some embodiments, X² comprises a side chain comprising —OH. In some embodiments, X² is S. In some embodiments, X² is Y. In some embodiments, X² is F.

Various amino acid residues may be utilized for X³. In some embodiments, X³ is T. In some embodiments, X³ is Q. In some embodiments, X³ is E. In some embodiments, X³ is R.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

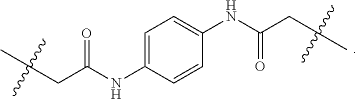 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is V. In some embodiments, X⁵ is I.

Various amino acid residues may be utilized for X⁶. For example, in some embodiments, the side chain of X⁶ comprises an aromatic group. In some embodiments, a cluster is enriched for amino acid residues comprising an aromatic group at X⁶. In some embodiments, X⁶ comprises a side chain comprising a polar group. In some embodiments, a cluster is enriched for amino acid residues comprising a polar group at X⁶. In some embodiments, X⁶ comprises a side chain comprising —OH. In some embodiments, X⁶ is Y.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. in some embodiments, the side chain of X⁹ comprises an aromatic group. In some embodiments, X⁹ is V. In some embodiments, X⁹ is F. In some embodiments, X⁹ is I. In some embodiments, X⁹ is L.

Various amino acid residues may be utilized for X¹⁰. In some embodiments, X¹⁰ is M. In some embodiments, X¹⁰ is F.

Various amino acid residues may be utilized for X¹². In some embodiments, X¹² is E. In some embodiments, X¹² is D.

Various amino acid residues may be utilized for X¹³. In some embodiments, X¹³ is D.

Various amino acid residues may be utilized for X¹⁴.

In some embodiments, an agent described herein, e.g., a stapled peptide, interacts with one or more (e.g., 1, 2, 3, 4, 5, 6, 7, 8, 9, 10, 11, 12, 13, 14, 15, or 16) or all of the following amino acid residues of SEQ ID NO: 2 or amino acid residues corresponding thereto: Glu506, Phe509, Ser510, Leu524, Arg525, Leu528, Tyr530, Val531, Met534, Leu538, Trp558, His562, Gly563, Gly563, Gly563, and Leu565. In some embodiments, it interacts with Glu506 of SEQ ID NO: 2 or an amino acid residue corresponding thereto. In some embodiments, it interacts with Phe509 of SEQ ID NO: 2 or an amino acid residue corresponding thereto. In some embodiments, it interacts with Ser510 of SEQ ID NO: 2 or an amino acid residue corresponding thereto. In some embodiments, it interacts with Leu524 of SEQ ID NO: 2 or an amino acid residue corresponding thereto. In some embodiments, it interacts with Arg525 of SEQ ID NO: 2 or an amino acid residue corresponding thereto. In some embodiments, it interacts with Leu528 of SEQ ID NO: 2 or an amino acid residue corresponding thereto. In some embodiments, it interacts with Tyr530 of SEQ ID NO: 2 or an amino acid residue corresponding thereto. In some embodiments, it interacts with Val531 of SEQ ID NO: 2 or an amino acid residue corresponding thereto. In some embodiments, it interacts with Met534 of SEQ ID NO: 2 or an amino acid residue corresponding thereto. In some embodiments, it interacts with Leu538 of SEQ ID NO: 2 or an amino acid residue corresponding thereto. In some embodiments, it interacts with Trp558 of SEQ ID NO: 2 or an amino acid residue corresponding thereto. In some embodiments, it interacts with His562 of SEQ ID NO: 2 or an amino acid residue corresponding thereto. In some embodiments, it interacts with Gly563 of SEQ ID NO: 2 or an amino acid residue corresponding thereto. In some embodiments, it interacts with Gly563 of SEQ ID NO: 2 or an amino acid residue corresponding thereto. In some embodiments, it interacts with Gly563 of SEQ ID NO: 2 or an amino acid residue corresponding thereto. In some embodiments, it interacts with Leu565 of SEQ ID NO: 2 or an amino acid residue corresponding thereto.

In some embodiments, an agent comprising a cluster 47 sequence interacts with RNF31 UBA domain.

### Cluster C48

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C33 as described herein. In some embodiments, an agent comprises X⁴⁸X²X³X⁴X⁵X⁶X⁷X⁸X⁹X¹⁰X¹¹X¹²X¹³X¹⁴, wherein each of X⁴⁸, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴⁸X²X³X⁴X⁵X⁶X⁷X⁸X⁹X¹⁰X¹¹X¹²X¹³X¹⁴,

wherein:
each of X⁴⁸, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising a basic group or an aromatic group;
X¹⁰ comprises a side chain comprising an aromatic group;
X¹² comprises a hydrophobic side chain; and
X¹³ comprises a hydrophobic side chain.

Various amino acid residues may be utilized for X⁴⁸. In some embodiments, X⁴⁸ is R.

Various amino acid residues may be utilized for X². In some embodiments, X² is E. In some embodiments, X² is E.

Various amino acid residues may be utilized for X³. For example, in some embodiments, X³ comprises a side chain comprising a basic group. In some embodiments, a cluster is enriched for amino acid residues comprising a basic group at X³. In some embodiments, the side chain of X³ comprises an aromatic group. In some embodiments, a cluster is enriched for amino acid residues comprising an aromatic group at X³. In some embodiments, X³ is H.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

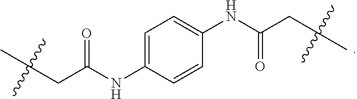 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ is D. In some embodiments, X⁵ is T.

Various amino acid residues may be utilized for X⁶. In some embodiments, X⁶ is A. In some embodiments, X⁶ is S.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ is A.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ comprises an aromatic group. In some embodiments, X¹⁰ is W. In some embodiments, X¹⁰ is F.

Various amino acid residues may be utilized for X¹². In some embodiments, X¹² is Y. In some embodiments, X¹² is W. In some embodiments, X¹² is H.

Various amino acid residues may be utilized for X¹³. For example, in some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, X¹³ is L. In some embodiments, X¹³ is I. In some embodiments, X¹³ is V.

Various amino acid residues may be utilized for X¹¹⁴. For example, in some embodiments, X¹⁴ comprises a hydrophobic side chain. In some embodiments, a cluster is enriched for amino acid residues comprising a hydrophobic side chain at X¹⁴. In some embodiments, the side chain of X¹⁴ is C₁₋₆ aliphatic. In some embodiments, the side chain of X¹⁴ is C₁₋₆ alkyl. In some embodiments, X¹⁴ is L. In some embodiments, X¹⁴ is M. In some embodiments, X¹⁴ is I.

In some embodiments, an agent comprising a cluster 48 sequence interacts with RNF31 UBA domain.

### CDK2

In some embodiments, the present disclosure provides agents that can bind to and/or modulate activities of CDK2. In some embodiments, provided stapled peptides selectively bind CDK2 or CDK2 in complex with its partner, e.g., CyclinE1, over CDK1 or CDK1 in complex with its partner, e.g., CyclinA2 (C51 and C52). In some embodiments, a stapled peptide does not compete with ATP. In some embodiments, a stapled peptide bind to CDK2 in the presence of other ATP-competitive CDK2-binding agents, e.g., proteins, Dinaciclib, Zotiraciclib, etc. In some embodiments, a stapled peptide does not inhibit the kinase activity of CDK2. In some embodiments, a stapled peptide does not inhibit the kinase activity of CDK2 in a luminescence-based assay that detects ADP production.

### Cluster C51

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C51 as described herein. In some embodiments, an agent comprises X⁵¹X²X³X⁴X⁵X⁶X⁷X⁸X⁹X¹⁰X¹¹X¹²X¹³X¹⁴, wherein each of X⁵¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁵¹X²X³X⁴X⁵X⁶X⁷X⁸X⁹X¹⁰X¹¹X¹²X¹³X¹⁴,

wherein:
each of X⁵¹, X², X³, X⁴, X⁵, X⁶, X⁷, X⁸, X⁹, X¹⁰, X¹¹, X¹², X¹³, and X¹⁴ is independently an amino acid residue;
X¹ comprises a side chain comprising an aromatic group;
X² comprises a side chain comprising an aromatic group;
X⁶ comprises a hydrophobic side chain;
X⁹ comprises a hydrophobic side chain; and
X¹⁰ comprises a side chain comprising an acidic group.

Various amino acid residues may be utilized for X⁵¹. For example, in some embodiments, X⁵¹ comprises a hydrophobic side chain. In some embodiments, a cluster is enriched for amino acid residues comprising a hydrophobic side chain at X⁵¹. In some embodiments, the side chain of X⁵¹ comprises an aromatic group. In some embodiments, a cluster is enriched for amino acid residues comprising an aromatic group at X⁵¹. In some embodiments, X⁵¹ is W.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² comprises an aromatic group. In some embodiments, a cluster is enriched for amino acid residues comprising an aromatic group at X². In some embodiments, X² is W. In some embodiments, X² is Y

Various amino acid residues may be utilized for X³. For example, in some embodiments, X³ is R. In some embodiments, X³ is V. In some embodiments, X³ is T. In some embodiments, X³ is A. In some embodiments, X³ is N.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

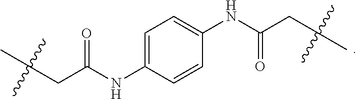 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is I. In some embodiments, X⁵ is V. In some embodiments, X⁵ is A. In some embodiments, the side chain of X⁵ comprises a polar group. In some embodiments, a cluster is enriched for amino acid residues comprising a polar group at X⁵. In some embodiments, X⁵ comprises a side chain comprising —OH. In some embodiments, X⁵ is S. In some embodiments, X⁵ is T.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, X⁶ is I. In some embodiments, X⁶ is V.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ is C₁₋₆ aliphatic. In some embodiments, the side chain of X⁹ is C₁₋₆ alkyl. In some embodiments, X⁹ is I. In some embodiments, X⁹ is L. In some embodiments, X⁹ is M. In some embodiments, X⁹ is V.

Various amino acid residues may be utilized for X¹⁰. For example, in some embodiments, X¹⁰ comprises a side chain comprising an acidic group. In some embodiments, a cluster is enriched for amino acid residues whose side chains comprising acidic groups at X¹⁰. In some embodiments, X¹⁰ comprises a side chain comprising —COOH. In some embodiments, X¹⁰ is E. In some embodiments, X¹⁰ is D. In some embodiments, X¹⁰ comprises a side chain comprising a polar group. In some embodiments, X¹⁰ is Q.

Various amino acid residues may be utilized for X¹². For example, in some embodiments, X¹² comprises a side chain comprising an acidic group. In some embodiments, a cluster is enriched for amino acid residues whose side chains comprising acidic groups at X¹². In some embodiments, X¹² comprises a side chain comprising —COOH. In some embodiments, X¹² is E. In some embodiments, X¹² comprises a side chain comprising a basic group. In some embodiments, a cluster is enriched for amino acid residues whose side chains comprising a basic groups at X¹². In some embodiments, X¹² is H. In some embodiments, X¹² is D. In some embodiments, X¹² comprises a side chain comprising a polar group. In some embodiments, X¹² is Q. In some embodiments, X¹² is S.

Various amino acid residues may be utilized for X¹³. For example, in some embodiments, X¹³ comprises a side chain comprising an acidic group. In some embodiments, a cluster is enriched for amino acid residues whose side chains comprising acidic groups at X¹³. In some embodiments, X¹³ comprises a side chain comprising —COOH. In some embodiments, X¹³ is D. In some embodiments, X¹³ is E. In some embodiments, X¹³ comprises a side chain comprising a polar group. In some embodiments, X¹³ is T. In some embodiments, X¹³ is S.

Various amino acid residues may be utilized for X¹⁴. For example, in some embodiments, X¹⁴ comprises a side chain comprising a polar group. In some embodiments, a cluster is enriched for amino acid residues comprising a polar group at X¹⁴. In some embodiments, X¹⁴ comprises a side chain comprising —OH. In some embodiments, X¹⁴ is T. In some embodiments, X¹⁴ is S. In some embodiments, X¹⁴ is Y. In some embodiments, X¹⁴ is Q. In some embodiments, X¹⁴ is V.

In some embodiments, an agent described herein, e.g., a stapled peptide, interacts with one or more (1, 2, 3, 4, 5, 6, 7, 8, 9, 10, 11, 12, 13, 14, 15, 16, or 17) or all of the following amino acid residues of SEQ ID NO: 3 or amino acid residues corresponding thereto: Gln5, Val7, Tyr19, Ala21, Asn23, Glu28, Val30, Leu32, Lys34, Arg36, Leu67, Asp68, Ile70, Thr72, Lys75, Tyr77, and Val79. In some embodiments, it interacts with Gln5 of SEQ ID NO: 3 or an amino acid residue corresponding thereto. In some embodiments, it interacts with Val7 of SEQ ID NO: 3 or an amino acid residue corresponding thereto. In some embodiments, it interacts with Tyr19 of SEQ ID NO: 3 or an amino acid residue corresponding thereto. In some embodiments, it interacts with Ala21 of SEQ ID NO: 3 or an amino acid residue corresponding thereto. In some embodiments, it interacts with Asn23 of SEQ ID NO: 3 or an amino acid residue corresponding thereto. In some embodiments, it interacts with Glu28 of SEQ ID NO: 3 or an amino acid residue corresponding thereto. In some embodiments, it interacts with Val30 of SEQ ID NO: 3 or an amino acid residue corresponding thereto. In some embodiments, it interacts with Leu32 of SEQ ID NO: 3 or an amino acid residue corresponding thereto. In some embodiments, it interacts with Lys34 of SEQ ID NO: 3 or an amino acid residue corresponding thereto. In some embodiments, it interacts with Arg36 of SEQ ID NO: 3 or an amino acid residue corresponding thereto. In some embodiments, it interacts with Leu67 of SEQ ID NO: 3 or an amino acid residue corresponding thereto. In some embodiments, it interacts with Asp68 of SEQ ID NO: 3 or an amino acid residue corresponding thereto. In some embodiments, it interacts with Ile70 of SEQ ID NO: 3 or an amino acid residue corresponding thereto. In some embodiments, it interacts with Thr72 of SEQ ID NO: 3 or an amino acid residue corresponding thereto. In some embodiments, it interacts with Lys75 of SEQ ID NO: 3 or an amino acid residue corresponding thereto. In some embodiments, it interacts with Tyr77 of SEQ ID NO: 3 or an amino acid residue corresponding thereto. In some embodiments, it interacts with Val79 of SEQ ID NO: 3 or an amino acid residue corresponding thereto.

(SEQ ID NO: 3)
MENFQKVEKIGEGTYGVVYKARNKLTGEVVALKKIRLDTETEGVPSTAI
REISLLKELNHPNIVKLLDVIHTENKLYLVFEFLHQDLKKFMDASALTG
IPLPLIKSYLFQLLQGLAFCHSHRVLHRDLKPQNLLINTEGAIKLADFG
LARAFGVPVRTYTHEVVTLWYRAPEILLGCKYYSTAVDIWSLGCIFAEM
VTRRALFPGDSEIDQLFRIFRTLGTPDEVVWPGVTSMPDYKPSFPKWAR
QDFSKVVPPLDEDGRSLLSQMLHYDPNKRISAKAALAHPFFQDVTKPVP
HLRL

In some embodiments, an agent comprising a cluster 51 sequence demonstrates selectivity for CDK2 and a CDK2-CCNE1 complex over CDK1 or a CDK1-CCNA2 complex.

### Cluster C52

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C52 as described herein. In some embodiments, an agent comprises X⁵²X²X³X⁴X⁵X⁶X⁷X⁸X⁹X¹⁰X¹¹X¹²X¹³X¹⁴, wherein each of X⁵², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁵²X²X³X⁴X⁵X⁶X⁷X⁸X⁹X¹⁰X¹¹X¹²X¹³X¹⁴,

wherein:
each of X⁵², X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain;
X⁸ comprises a side chain comprising an aromatic group; and
X⁹ comprises a side chain comprising an aromatic group.

Various amino acid residues may be utilized for X⁵². For example, in some embodiments, X⁵² is P. In some embodiments, X⁵² is absent.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² is C₁₋₆ aliphatic. In some embodiments, the side chain of X² is C₁₋₆ alkyl. In some embodiments, the side chain of X² comprises an aromatic group. In some embodiments, X² is P. In some embodiments, X² is F. In some embodiments, X² is M. In some embodiments, X² is Y.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is E. In some embodiments, X³ comprises a hydrophobic side chain. In some embodiments, a cluster is enriched for amino acid residues comprising a hydrophobic side chain at X³. In some embodiments, the side chain of X³ is C₁₋₆ aliphatic. In some embodiments, the side chain of X³ is C₁₋₆ alkyl. In some embodiments, the side chain of X³ comprises an aromatic group. In some embodiments, X³ is P. In some embodiments, X³ is A. In some embodiments, X³ is Y.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

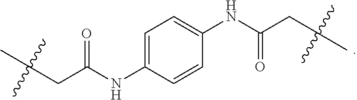 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is L. In some embodiments, X⁵ is V. In some embodiments, X⁵ is M.

Various amino acid residues may be utilized for X⁶. For example, in some embodiments, X⁶ comprises a side chain comprising an acidic group. In some embodiments, a cluster is enriched for amino acid residues whose side chains comprise acidic groups at X⁶. In some embodiments, X⁶ comprises a side chain comprising —COOH. In some embodiments, X⁶ is D. In some embodiments, X⁶ is E. In some embodiments, the side chain of X⁶ comprises a polar group. In some embodiments, a cluster is enriched for amino acid residues comprising a polar group at X⁶. In some embodiments, X⁶ comprises a side chain comprising —OH. In some embodiments, X⁶ is S.

Various amino acid residues may be utilized for X⁷. In some embodiments, X⁷ is A.

Various amino acid residues may be utilized for X⁸. For example, in some embodiments, X⁸ comprises a hydrophobic side chain. In some embodiments, a cluster is enriched for amino acid residues comprising a hydrophobic side chain at X⁸. In some embodiments, the side chain of X⁸ comprises an aromatic group. In some embodiments, X⁸ is F. In some embodiments, X⁸ is Y.

Various amino acid residues may be utilized for X⁹. For example, in some embodiments, X⁹ comprises a hydrophobic side chain. In some embodiments, a cluster is enriched for amino acid residues comprising a hydrophobic side chain at X⁹. In some embodiments, the side chain of X⁹ comprises an aromatic group. In some embodiments, X⁹ is F.

Various amino acid residues may be utilized for X¹⁰. For example, in some embodiments, the side chain of X¹⁰ comprises a polar group. In some embodiments, a cluster is enriched for amino acid residues comprising a polar group at X¹⁹. In some embodiments, X¹⁰ comprises a side chain comprising —OH. In some embodiments, X¹⁰ is S. In some embodiments, X¹⁰ is T. In some embodiments, X¹⁰ is A.

Various amino acid residues may be utilized for X¹². For example, in some embodiments, X¹² is V. In some embodiments, X¹² is absent.

Various amino acid residues may be utilized for X¹³. In some embodiments, X¹³ is absent.

Various amino acid residues may be utilized for X¹⁴. In some embodiments, X¹⁴ is absent.

In some embodiments, an agent described herein, e.g., a stapled peptide, interacts with one or more (e.g., 1, 2, 3, 4, 5, 6, 7, 8, 9, 10, 11, 12, 13, 14, 15, or 16) or all of the following amino acid residues of SEQ ID NO: 3 or amino acid residues corresponding thereto: His84, Gln85, Lys89, Phe90, Ala93, Ser94, Thr97, Ile99, Pro100, Leu103, Tyr107, Ile135, Thr137, Thr137, Thr137, and Gly139. In some embodiments, it interacts with His84 of SEQ ID NO: 3 or an amino acid residue corresponding thereto. In some embodiments, it interacts with Gln85 of SEQ ID NO: 3 or an amino acid residue corresponding thereto. In some embodiments, it interacts with Lys89 of SEQ ID NO: 3 or an amino acid residue corresponding thereto. In some embodiments, it interacts with Phe90 of SEQ ID NO: 3 or an amino acid residue corresponding thereto. In some embodiments, it interacts with Ala93 of SEQ ID NO: 3 or an amino acid residue corresponding thereto. In some embodiments, it interacts with Ser94 of SEQ ID NO: 3 or an amino acid residue corresponding thereto. In some embodiments, it interacts with Thr97 of SEQ ID NO: 3 or an amino acid residue corresponding thereto. In some embodiments, it interacts with Ile99 of SEQ ID NO: 3 or an amino acid residue corresponding thereto. In some embodiments, it interacts with Pro100 of SEQ ID NO: 3 or an amino acid residue corresponding thereto. In some embodiments, it interacts with Leu103 of SEQ ID NO: 3 or an amino acid residue corresponding thereto. In some embodiments, it interacts with Tyr107 of SEQ ID NO: 3 or an amino acid residue corresponding thereto. In some embodiments, it interacts with Ile135 of SEQ ID NO: 3 or an amino acid residue corresponding thereto. In some embodiments, it interacts with Thr137 of SEQ ID NO: 3 or an amino acid residue corresponding thereto. In some embodiments, it interacts with Thr137 of SEQ ID NO: 3 or an amino acid residue corresponding thereto. In some embodiments, it interacts with Thr137 of SEQ ID NO: 3 or an amino acid residue corresponding thereto. In some embodiments, it interacts with Gly139 of SEQ ID NO: 3 or an amino acid residue corresponding thereto.

### PPIA

In some embodiments, the present disclosure provides agents that can bind to and/or modulate activities of PPIA. In some embodiments, a stapled peptide competes with CsA for PPIA interaction.

### Cluster C53

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C53 as described herein. In some embodiments, an agent comprises X⁵³X²X³X⁴X⁵X⁶X⁷X⁸X⁹X¹⁰X¹¹X¹²X¹³X¹⁴, wherein each of X⁵³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁵³X²X³X⁴X⁵X⁶X⁷X⁸X⁹X¹⁰X¹¹X¹²X¹³X¹⁴,

wherein:
each of X⁵³, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising an acidic group;
X⁵ comprises a side chain comprising a basic or aromatic group;
X⁶ comprises a hydrophobic side chain; and
X⁹ comprises a side chain comprising an aromatic group or a polar group.

Various amino acid residues may be utilized for X⁵³. For example, in some embodiments, X⁵³ comprises a side chain comprising a basic group. In some embodiments, a cluster is enriched for amino acid residues comprising a basic group at X⁵³. In some embodiments, X⁵³ is R. In some embodiments, the side chain of X⁵³ comprises a polar group. In some embodiments, a cluster is enriched for amino acid residues comprising a polar group at X⁵³. In some embodiments, X⁵³ is T. In some embodiments, X⁵³ is Q. In some embodiments, X⁵³ is S. In some embodiments, X⁵³ is A. In some embodiments, X⁵³ is N.

Various amino acid residues may be utilized for X². For example, in some embodiments, X² is Q. In some embodiments, X² is H. In some embodiments, X² is Y. In some embodiments, X² is T. In some embodiments, X² is A.

Various amino acid residues may be utilized for X³. For example, in some embodiments, X³ comprises a side chain comprising an acidic group. In some embodiments, a cluster is enriched for amino acid residues whose side chains comprise acidic groups at X³. In some embodiments, X³ comprises a side chain comprising —COOH. In some embodiments, X³ is D. In some embodiments, the side chain of X³ comprises a polar group. In some embodiments, a cluster is enriched for amino acid residues comprising a polar group at X³. In some embodiments, X³ comprises a side chain comprising —OH. In some embodiments, X³ is S. In some embodiments, X³ is N.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

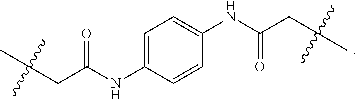 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues whose side chains comprise basic groups at X⁵. In some embodiments, X⁵ is H. In some embodiments, X⁵ is R. In some embodiments, X⁵ is Q.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, X⁶ is V. In some embodiments, X⁶ is I.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ comprises an aromatic group. In some embodiments, a cluster is enriched for amino acid residues comprising an aromatic group at X⁹. In some embodiments, X⁹ is Y. In some embodiments, X⁹ is W. In some embodiments, X⁹ is F.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C₁₋₆ aliphatic. In some embodiments, the side chain of X¹⁰ is C₁₋₆ alkyl. In some embodiments, the side chain of X¹⁰ comprises an aromatic group. In some embodiments, X¹⁰ is H. In some embodiments, X¹⁰ is W. In some embodiments, X¹⁰ is F. In some embodiments, X¹⁰ is L. In some embodiments, X¹⁰ is Q.

Various amino acid residues may be utilized for X¹². For example, in some embodiments, X¹² comprises a hydrophobic side chain. In some embodiments, a cluster is enriched for amino acid residues comprising a hydrophobic side chain at X¹². In some embodiments, the side chain of X¹² is C_{1 6} aliphatic. In some embodiments, the side chain of X¹² is C₁₋₆ alkyl. In some embodiments, the side chain of X¹² comprises an aromatic group. In some embodiments, X¹² is W. In some embodiments, X¹² is Y. In some embodiments, X¹² is I. In some embodiments, X¹² is H.

Various amino acid residues may be utilized for X¹³. For example, in some embodiments, X¹³ is Q. In some embodiments, X¹³ is H. In some embodiments, X¹³ is F. In some embodiments, X¹³ is W.

Various amino acid residues may be utilized for X¹⁴. For example, in some embodiments, X¹⁴ is R. In some embodiments, X¹⁴ is Q.

In some embodiments, an agent described herein, e.g., a stapled peptide, interacts with one or more (e.g., 1, 2, 3, 4, 5, 6, 7, 8, 9, 10, 11, 12, 13, or 14) or all of the following amino acid residues of SEQ ID NO: 4 or amino acid residues corresponding thereto: His54, Ile57, Phe60, Met61, Gln63, Thr73, Lys82, Ala101, Ala103, Gln111, Phe113, Trp121, Leu122, and His126. In some embodiments, it interacts with His54 of SEQ ID NO: 4 or an amino acid residue corresponding thereto. In some embodiments, it interacts with Ile57 of SEQ ID NO: 4 or an amino acid residue corresponding thereto. In some embodiments, it interacts with Phe60 of SEQ ID NO: 4 or an amino acid residue corresponding thereto. In some embodiments, it interacts with Met61 of SEQ ID NO: 4 or an amino acid residue corresponding thereto. In some embodiments, it interacts with Gln63 of SEQ ID NO: 4 or an amino acid residue corresponding thereto. In some embodiments, it interacts with Thr73 of SEQ ID NO: 4 or an amino acid residue corresponding thereto. In some embodiments, it interacts with Lys82 of SEQ ID NO: 4 or an amino acid residue corresponding thereto. In some embodiments, it interacts with Ala101 of SEQ ID NO: 4 or an amino acid residue corresponding thereto. In some embodiments, it interacts with Ala103 of SEQ ID NO: 4 or an amino acid residue corresponding thereto. In some embodiments, it interacts with Gln111 of SEQ ID NO: 4 or an amino acid residue corresponding thereto. In some embodiments, it interacts with Phe113 of SEQ ID NO: 4 or an amino acid residue corresponding thereto. In some embodiments, it interacts with Trp121 of SEQ ID NO: 4 or an amino acid residue corresponding thereto. In some embodiments, it interacts with Leu122 of SEQ ID NO: 4 or an amino acid residue corresponding thereto. In some embodiments, it interacts with His126 of SE ID NO: 4 or an amino acid residue corresponding thereto.

(SEQ ID NO: 4)
MVNPTVFFDIAVDGEPLGRVSFELFADKVPKTAENFRALSTGEKGFGYK
GSCFHRIIPGFMCQGGDFTRHNGTGGKSIYGEKFEDENFILKHTGPGIL
SMANAGPNINGSQFFICTAKTEWLDGKHVVFGKVKEGMNIVEAMERFGS
RNGKTSKKITIADCGQLE

In some embodiments, an agent comprising a cluster 53 sequence competes with cyclosporine for PPIA binding. In some embodiments, an agent comprising a cluster 53 sequence inhibit PPIA peptidyl-prolyl cis-trans isomerase activity. In some embodiments, an agent comprising a cluster 53 sequence inhibit PPIA peptidyl-prolyl cis-trans isomerase activity but with higher IC₅₀ than CsA.

### Cluster C54

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C54 as described herein. In some embodiments, an agent comprises X⁵⁴X²X³X⁴X⁵X⁶X⁷X⁸X⁹X¹⁰X¹¹X¹²X¹³X¹⁴, wherein each of X⁵⁴, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁵⁴X²X³X⁴X⁵X⁶X⁷X⁸X⁹X¹⁰X¹¹X¹²X¹³X¹⁴,

wherein:
each of X⁵⁴, X², X³, X⁴, X⁵, X⁶, X⁷, X⁸, X⁹, X¹⁰, X¹¹, X¹², X¹³, and X¹⁴ is independently an amino acid residue;
X⁵ comprises a side chain comprising an aromatic group or a basic group;
X⁶ comprises a hydrophobic side chain;
X⁸ comprises a hydrophobic side chain; and
X⁹ comprises a side chain comprising an aromatic group.

Various amino acid residues may be utilized for X⁵⁴. For example, in some embodiments, X⁵⁴ is P. In some embodiments, X⁵⁴ is absent.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² is C₁₋₆ aliphatic. In some embodiments, the side chain of X² is C₁₋₆ alkyl. In some embodiments, the side chain of X² comprises an aromatic group. In some embodiments, X² is P. In some embodiments, X² is Y. In some embodiments, X² is H.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, the side chain of X³ comprises a polar group. In some embodiments, X³ is N. In some embodiments, X³ is T. In some embodiments, X³ is Y.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

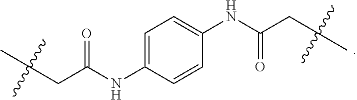 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues comprising a basic group at X⁵. In some embodiments, the side chain of X⁵ comprises an aromatic group. In some embodiments, a cluster is enriched for amino acid residues comprising an aromatic group at X⁵. In some embodiments, X⁵ is H.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, X⁶ is I. In some embodiments, X⁶ is V. In some embodiments, X⁶ is L.

Various amino acid residues may be utilized for X⁷. In some embodiments, X⁷ is A. In some embodiments, X⁷ is R.

Various amino acid residues may be utilized for X⁸. In some embodiments, X⁸ is A.

Various amino acid residues may be utilized for X⁹. For example, in some embodiments, the side chain of X⁹ comprises an aromatic group. In some embodiments, a cluster is enriched for amino acid residues comprising an aromatic group at X⁹. In some embodiments, X⁹ is Y. In some embodiments, X⁹ is W. In some embodiments, X⁹ is F.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹ is C₁₋₆ aliphatic. In some embodiments, the side chain of X¹⁰ is C₁₋₆ alkyl. In some embodiments, the side chain of X¹⁰ comprises an aromatic group. In some embodiments, X¹⁰ is Y. In some embodiments, X¹⁰ is M. In some embodiments, X¹⁰ is F. In some embodiments, X¹⁰ is V.

Various amino acid residues may be utilized for X¹². For example, in some embodiments, X¹² is H.

Various amino acid residues may be utilized for X¹³. In some embodiments, X¹³ is absent.

Various amino acid residues may be utilized for X¹⁴. In some embodiments, X¹⁴ is absent.

In some embodiments, an agent described herein, e.g., a stapled peptide, interacts with one or more (e.g., 1, 2, 3, 4, 5, 6, 7, 8, 9, 10, 11, 12, 13, or 14) or all of the following amino acid residues of SEQ ID NO: 4 or amino acid residues corresponding thereto: His54, Ile57, Phe60, Met61, Gln63, Thr73, Lys82, Ala101, Ala103, Gln111, Phe113, Trp121, Leu122, and His126. In some embodiments, it interacts with His54 of SEQ ID NO: 4 or an amino acid residue corresponding thereto. In some embodiments, it interacts with Ile57 of SEQ ID NO: 4 or an amino acid residue corresponding thereto. In some embodiments, it interacts with Phe60 of SEQ ID NO: 4 or an amino acid residue corresponding thereto. In some embodiments, it interacts with Met61 of SEQ ID NO: 4 or an amino acid residue corresponding thereto. In some embodiments, it interacts with Gln63 of SEQ ID NO: 4 or an amino acid residue corresponding thereto. In some embodiments, it interacts with Thr73 of SEQ ID NO: 4 or an amino acid residue corresponding thereto. In some embodiments, it interacts with Lys82 of SEQ ID NO: 4 or an amino acid residue corresponding thereto. In some embodiments, it interacts with Ala101 of SEQ ID NO: 4 or an amino acid residue corresponding thereto. In some embodiments, it interacts with Ala103 of SEQ ID NO: 4 or an amino acid residue corresponding thereto. In some embodiments, it interacts with Gln11I of SEQ ID NO: 4 or an amino acid residue corresponding thereto. In some embodiments, it interacts with Phe113 of SEQ ID NO: 4 or an amino acid residue corresponding thereto. In some embodiments, it interacts with Trp121 of SEQ ID NO: 4 or an amino acid residue corresponding thereto. In some embodiments, it interacts with Leu122 of SEQ ID NO: 4 or an amino acid residue corresponding thereto. In some embodiments, it interacts with His126 of SEQ ID NO: 4 or an amino acid residue corresponding thereto.

In some embodiments, an agent comprising a cluster 54 sequence competes with cyclosporine for PPIA binding. In some embodiments, an agent comprising a cluster 54 sequence inhibit PPIA peptidyl-prolyl cis-trans isomerase activity. In some embodiments, an agent comprising a cluster 54 sequence inhibit PPIA peptidyl-prolyl cis-trans isomerase activity but with higher IC₅₀ than CsA.

### PD-Li

In some embodiments, the present disclosure provides agents that can bind to and/or modulate activities of PD-L1. In some embodiments, a stapled peptide reduces interaction between PD-L1 and PD1. In some embodiments, a stapled peptide induces PD-L1 dimerization. In some embodiments, a stapled peptide binds to extracellular domain of PD-L1.

### Cluster C61

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C61 as described herein. In some embodiments, an agent comprises X⁶¹X²X³X⁴X⁵X⁶X⁷X⁸X⁹X⁰X¹¹X¹²X¹³X¹⁴, wherein each of X⁶¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁶¹X³X⁴X⁵X⁶X⁷X⁸X⁹X¹¹X¹²X¹³X¹⁴,

wherein:
each of X⁶¹, X³, X⁴, X⁵, X⁶, X⁷, X⁸, X⁹, X°, X¹, X¹², X¹³, and X¹⁴ is independently an amino acid residue;
X⁶¹ comprises a side chain comprising an aromatic group;
X⁶ comprises a hydrophobic side chain and/or a side chain comprising an aromatic group; and
X⁹ comprises a side chain comprising a basic group.

Various amino acid residues may be utilized for X⁶¹. For example, in some embodiments, X⁶¹ is L.

Various amino acid residues may be utilized for X². For example, in some embodiments, X² comprises a hydrophobic side chain. In some embodiments, a cluster is enriched for amino acid residues comprising a hydrophobic side chain at X². In some embodiments, the side chain of X² comprises an aromatic group. In some embodiments, a cluster is enriched for amino acid residues comprising an aromatic group at X². In some embodiments, X² is W.

Various amino acid residues may be utilized for X³. For example, in some embodiments, X³ is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

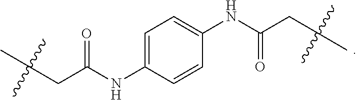 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, X⁵ is V.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ comprises an aromatic group. In some embodiments, a cluster is enriched for amino acid residues comprising an aromatic group at X⁶. In some embodiments, X⁶ is F.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising a basic group. In some embodiments, a cluster is enriched for amino acid residues comprising a basic group at X⁹. In some embodiments, X⁹ is R.

Various amino acid residues may be utilized for X¹⁰. For example, in some embodiments, X¹⁰ is S.

Various amino acid residues may be utilized for X¹². For example, in some embodiments, X¹² is Y.

Various amino acid residues may be utilized for X¹³. In some embodiments, side chain of X¹³ comprises an acidic group, e.g., —COOH. In some embodiments, X¹³ is E.

Various amino acid residues may be utilized for X¹⁴. For example, in some embodiments, side chain of X¹⁴ comprises an acidic group, e.g., —COOH. In some embodiments, X¹⁴ is E. In some embodiments, X¹⁴ is absent.

In some embodiments, an agent described herein, e.g., a stapled peptide, interacts with one or more (1, 2, 3, 4, 5, 6, 7, 8, 9, 10, 11, 12, 13, or 14) or all of the following amino acid residues of SEQ ID NO: 5 or amino acid residues corresponding thereto: Ile54, Tyr56, Glu58, Asp61, Asn63, Gln66, Val68, Val76, His78, Arg113, Met115, Ser117, Ala121, and Tyr123. In some embodiments, it interacts with Ile54 of SEQ ID NO: 5 or an amino acid residue corresponding thereto. In some embodiments, it interacts with Tyr56 of SEQ ID NO: 5 or an amino acid residue corresponding thereto. In some embodiments, it interacts with Glu58 of SEQ ID NO: 5 or an amino acid residue corresponding thereto. In some embodiments, it interacts with Asp61 of SEQ ID NO: 5 or an amino acid residue corresponding thereto. In some embodiments, it interacts with Asn63 of SEQ ID NO: 5 or an amino acid residue corresponding thereto. In some embodiments, it interacts with Gln66 of SEQ ID NO: 5 or an amino acid residue corresponding thereto. In some embodiments, it interacts with Val68 of SEQ ID NO: 5 or an amino acid residue corresponding thereto. In some embodiments, it interacts with Val76 of SEQ ID NO: 5 or an amino acid residue corresponding thereto. In some embodiments, it interacts with His78 of SEQ ID NO: 5 or an amino acid residue corresponding thereto. In some embodiments, it interacts with Arg113 of SEQ ID NO: 5 or an amino acid residue corresponding thereto. In some embodiments, it interacts with Met115 of SEQ ID NO: 5 or an amino acid residue corresponding thereto. In some embodiments, it interacts with Ser117 of SEQ ID NO: 5 or an amino acid residue corresponding thereto. In some embodiments, it interacts with Ala121 of SEQ ID NO: 5 or an amino acid residue corresponding thereto. In some embodiments, it interacts with Tyr123 of SEQ ID NO: 5 or an amino acid residue corresponding thereto.

(SEQ ID NO: 5)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET

### Cluster C62

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C62 as described herein. In some embodiments, an agent comprises X⁶²X²X³X⁴X⁵X⁶X⁷X⁸X⁹X¹⁰X¹¹X¹²X¹³X¹⁴, wherein each of X⁶², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁶²X²X³X⁴X⁵X⁶X⁷X⁸X⁹X¹⁰X¹¹X¹²X¹³X¹⁴,

wherein:
each of X⁶², X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising a polar group or an acidic group;
X⁶ comprises a hydrophobic side chain or a side chain comprising an aromatic group; and
X⁹ comprises a side chain comprising an aromatic group.

Various amino acid residues may be utilized for X⁶². For example, in some embodiments, X⁶² comprises a hydrophobic side chain. In some embodiments, a cluster is enriched for amino acid residues comprising a hydrophobic side chain at X⁶². In some embodiments, the side chain of 62 is C₁₋₆ aliphatic. In some embodiments, the side chain of X⁶² is C₁₋₆ alkyl. In some embodiments, X⁶² is I. In some embodiments, X⁶² is V. In some embodiments, the side chain of X⁶² comprises a polar group. In some embodiments, a cluster is enriched for amino acid residues comprising a polar group at X⁶². In some embodiments, X⁶² comprises a side chain comprising —OH. In some embodiments, X⁶² is S. In some embodiments, X⁶² is Y.

Various amino acid residues may be utilized for X². For example, in some embodiments, X² comprises a side chain comprising an acidic group. In some embodiments, a cluster is enriched for amino acid residues whose side chains comprising acidic groups at X². In some embodiments, X² comprises a side chain comprising —COOH. In some embodiments, X² is D. In some embodiments, X² is E.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH. In some embodiments, X³ is D. In some embodiments, X³ is E. In some embodiments, the side chain of X³ comprises a polar group. In some embodiments, a cluster is enriched for amino acid residues comprising a polar group at X³. In some embodiments, X³ comprises a side chain comprising —OH. In some embodiments, X³ is S. In some embodiments, X³ is T. In some embodiments, X³ is Y.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

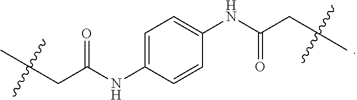 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues comprising a basic group at X⁵. In some embodiments, X⁵ is R. In some embodiments, X⁵ comprises a side chain comprising an aromatic group. In some embodiments, a cluster is enriched for amino acid residues comprising an aromatic group at X⁵. In some embodiments, X⁵ is W. In some embodiments, X⁵ comprises a hydrophobic side chain. In some embodiments, a cluster is enriched for amino acid residues comprising a hydrophobic side chain at X⁶². In some embodiments, the side chain of X⁶² is C_{1 6} aliphatic. In some embodiments, the side chain of X⁶² is C₁₋₆ alkyl. In some embodiments, X⁶² is A.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ comprises an aromatic group. In some embodiments, the side chain of X⁶ is C₁₋₆ aliphatic. In some embodiments, the side chain of X⁶ is C₁₋₆ alkyl. In some embodiments, X⁶ is W. In some embodiments, X⁶ is F. In some embodiments, X⁶ is V.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ comprises an aromatic group. In some embodiments, a cluster is enriched for amino acid residues comprising an aromatic group at X⁹. In some embodiments, X⁹ is F. In some embodiments, the side chain of X⁹ comprises a polar group. In some embodiments, a cluster is enriched for amino acid residues comprising a polar group at X⁹. In some embodiments, X⁹ comprises a side chain comprising —OH. In some embodiments, X⁹ is T. In some embodiments, X⁹ is Y.

Various amino acid residue may be utilized for X¹⁰. For example, in some embodiments, X¹⁰ is L.

Various amino acid residue may be utilized for X¹¹.

Various amino acid residues may be utilized for X¹². For example, in some embodiments, X¹² comprises a hydrophobic side chain. In some embodiments, a cluster is enriched for amino acid residues comprising a hydrophobic side chain at X¹². In some embodiments, the side chain of X¹² is C₁₋₆ aliphatic. In some embodiments, the side chain of X¹² is C₁₋₆ alkyl. In some embodiments, X¹² is M. In some embodiments, X¹² is A. In some embodiments, the side chain of X¹² comprises a basic group. In some embodiments, a cluster is enriched for amino acid residues comprising a side chain comprising a basic group at X¹². In some embodiments, X¹² is R.

Various amino acid residue may be utilized for X¹³. For example, in some embodiments, X¹³ is V.

Various amino acid residues may be utilized for X¹⁴. For example, in some embodiments, the side chain of X¹⁴ comprises a polar group. In some embodiments, a cluster is enriched for amino acid residues comprising a polar group at X¹⁴. In some embodiments, X¹⁴ comprises a side chain comprising —OH. In some embodiments, X¹⁴ is Y. In some embodiments, X¹⁴ is S.

In some embodiments, an agent described herein, e.g., a stapled peptide, interacts with one or more (1, 2, 3, 4, 5, 6, 7, 8, 9, 10, 11 or 12) or all of the following amino acid residues of SEQ ID NO: 5 or amino acid residues corresponding thereto: Ile54, Tyr56, Glu58, Asp61, Gln66, Val68, Arg113, Met115, Ser117, Ala121, Tyr123, and Arg125. In some embodiments, it interacts with Ile54 of SEQ ID NO: 5 or an amino acid residue corresponding thereto. In some embodiments, it interacts with Tyr56 of SEQ ID NO: 5 or an amino acid residue corresponding thereto. In some embodiments, it interacts with Glu58 of SEQ ID NO: 5 or an amino acid residue corresponding thereto. In some embodiments, it interacts with Asp61 of SEQ ID NO: 5 or an amino acid residue corresponding thereto. In some embodiments, it interacts with Gln66 of SEQ ID NO: 5 or an amino acid residue corresponding thereto. In some embodiments, it interacts with Val68 of SEQ ID NO: 5 or an amino acid residue corresponding thereto. In some embodiments, it interacts with Arg113 of SEQ ID NO: 5 or an amino acid residue corresponding thereto. In some embodiments, it interacts with Met115 of SEQ ID NO: 5 or an amino acid residue corresponding thereto. In some embodiments, it interacts with Ser117 of SEQ ID NO: 5 or an amino acid residue corresponding thereto. In some embodiments, it interacts with Ala121 of SEQ ID NO: 5 or an amino acid residue corresponding thereto. In some embodiments, it interacts with Tyr123 of SEQ ID NO: 5 or an amino acid residue corresponding thereto. In some embodiments, it interacts with Arg125 of SEQ ID NO: 5 or an amino acid residue corresponding thereto.

### HECT E3 Family

In some embodiments, the present disclosure provides agents that can bind to HECT E3 ligases. In some embodiments, provided agents can bind to WWP1. In some embodiments, provided agents can bind to WWP2. In some embodiments, agents comprising sequences of certain clusters, e.g., cluster C71, are capable of binding both an inactive form of WWP1 (e.g., WWP1^{WW-HECT}), a form of WWP1 that may assume active and inactive states (e.g., WWP1^{HECT}), and a form of WWP2 that may assume active and inactive states (e.g., WWP2^{HECT}). In some embodiments, agents comprising sequences of certain clusters, e.g., cluster C72, are capable of selectively binding to WWP2 (e.g., WWP2^{H}CT) over WWP1 (e.g., WWP1^{HECT} and/or WWP1^{WW-HECT}). In some embodiments, agents comprising sequences of certain clusters, e.g., cluster C73, are capable of binding an active form of WWP2 (e.g., WWP2^{HECT}) and/or selectively binding to an active form of WWP1 (e.g., WWP1^{HECT}) over an inactive form WWP1 (e.g., WWP1^{WW-HECT}). In some embodiments, stapled peptides comprising sequences of certain clusters, e.g., cluster C74, are capable of selectively binding to an inactive form of WWP1 (e.g., WWP1^{WW-HECT}) over an active form of WWP1 (e.g., WWP1^{HECT}) or an active form of WWP2 (e.g., WWP2^{HECT}) In some embodiments, agents comprising sequences of certain clusters, e.g., cluster C71 and cluster C73, are capable of binding to the HECT domain of WWP1. In some embodiments, agents comprising sequences of certain clusters, e.g., cluster C71, interact with the N-lobe of the HECT domain of WWP1. In some embodiments, agents comprising sequences of certain clusters, e.g., cluster C71 and cluster C73, interact with the N-lobe and the C-lobe of the HECT domain of WWP2. In some embodiments, agents comprising sequences of certain clusters, e.g., cluster C72, interact with the C-lobe of the HECT domain of WWP2. In some embodiments, the present disclosure provides methods for modulating a function or interaction of an HECT E3 ligases, comprising contacting the ligase with a provided agent.

### Cluster C71

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C71 as described herein. In some embodiments, an agent comprises X⁷¹X²X³X⁴X⁵X⁶X⁷X⁸X⁹X¹⁰X¹¹X¹²X¹³X¹⁴, wherein each of X⁷¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷¹X²X³X⁴X⁵X⁶X⁷X⁸X⁹X¹⁰X¹¹X¹²X¹³X¹⁴,

wherein:
each of X⁷¹, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain.

Various amino acid residues may be utilized for X⁷¹. For example, in some embodiments, X⁷¹ comprises a hydrophobic side chain. In some embodiments, a cluster is enriched for amino acid residues whose side chains comprise a hydrophobic side chain at X⁷¹. In some embodiments, X⁷¹ is M. In some embodiments, the side chain of X⁷¹ is aliphatic. In some embodiments, the side chain of X⁷¹ is C₁₋₆ alkyl. In some embodiments, X⁷¹ is A. In some embodiments, X⁷¹ is V. In some embodiments, X⁷¹ is I. In some embodiments, X⁷¹ is L. In some embodiments, X⁷¹ is Q.

Various amino acid residues may be utilized for X². In some embodiments, X² is M.

Various amino acid residues may be utilized for X³. For example, in some embodiments, X³ comprises a side chain comprising a basic group. In some embodiments, a cluster is enriched for amino acid residues whose side chains comprise a basic group at X³. In some embodiments, X³ is R. In some embodiments, X³ is H. In some embodiments, X³ comprises an acidic side chain. In some embodiments, a cluster is enriched for amino acid residues whose side chains comprise an acidic group at X³. In some embodiments, X³ comprises a side chain comprising —COOH. In some embodiments, X³ is D. In some embodiments, X³ comprises a side chain comprising a polar group. In some embodiments, a cluster is enriched for amino acid residues whose side chains comprise a polar group. In some embodiments, X³ comprises a side chain comprising a polar group. In some embodiments, X³ comprises a side chain comprising —OH. In some embodiments, X³ is S. In some embodiments, X³ is T. In some embodiments, X³ is Y.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

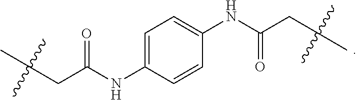 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a chain comprising a polar group. In some embodiments, a cluster is enriched for amino acid residues whose side chains comprise a polar group at X⁵. In some embodiments, X⁵ comprises a side chain comprising —OH. In some embodiments, X⁵ is S. In some embodiments, X⁵ is T. In some embodiments, X⁵ is M. In some embodiments, X⁵ comprises a side chain comprising a polar group. In some embodiments, a cluster is enriched for amino acid residues whose side chains comprise a polar group at X⁵. In some embodiments, X⁵ comprises a side chain comprising —OH. In some embodiments, X⁵ is T. In some embodiments, X⁵ is R.

Various amino acid residues may be utilized for X⁶. For example, in some embodiments, X⁶ comprises a side chain comprising a polar group. In some embodiments, a cluster is enriched for amino acid residues whose side chains comprise a basic polar at X⁶. In some embodiments, X⁶ comprises a side chain comprising —C(O)NH₂. In some embodiments, X⁶ is Q. In some embodiments, X⁶ is E.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising a basic group. In some embodiments, a cluster is enriched for amino acid residues comprising a basic group at X⁹. In some embodiments, X⁹ is R. In some embodiments, X⁹ is H. In some embodiments, X⁹ comprises a side chain comprising a polar group. In some embodiments, X⁹ comprises a side chain comprising —C(O)NH₂. In some embodiments, X⁹ is N. In some embodiments, X⁹ is Q. In some embodiments, X⁹ comprises a side chain comprising an aromatic group. In some embodiments, a cluster is enriched for amino acid residues whose side chains comprise an aromatic group at X⁹. In some embodiments, X⁹ is Y. In some embodiments, X⁹ is W. In some embodiments, X⁹ is F

Various amino acid residues may be utilized for X¹⁰. For example, in some embodiments, the side chain of X¹⁰ is C₁₋₆ aliphatic. In some embodiments, the side chain of X¹⁰ is C₁₋₆ alkyl. In some embodiments, X¹⁰ is I. In some embodiments, X¹⁰ is V. In some embodiments, X¹⁰ may be Y

Various amino acid residues may be utilized for X¹². For example, in some embodiments, X¹² comprises a side chain comprising a basic group. In some embodiments, a cluster is enriched for amino acid residues comprising a basic group at X¹². In some embodiments, X¹² is R. In some embodiments, X¹² is H. In some embodiments, X¹² comprises a side chain comprising an acidic group. In some embodiments, a cluster is enriched for amino acid residues comprising an acidic group at X¹². In some embodiments, X¹² comprises a side chain comprising —COOH. In some embodiments, X¹² is D. In some embodiments, X¹² is E. In some embodiments, X¹² comprises a side chain comprising a polar group. In some embodiments, a cluster is enriched for amino acid residues whose side chains comprise a polar group at X¹². In some embodiments, X¹² comprises a side chain comprising —OH. In some embodiments, X¹² is S. In some embodiments, X¹² may be F.

Various amino acid residues may be utilized for X¹³. For example, in some embodiments, X¹³ comprises a hydrophobic side chain. In some embodiments, a cluster is enriched for amino acid residues comprising a hydrophobic side chain at X¹³. In some embodiments, the side chain of X¹³ is C₁₋₆ aliphatic. In some embodiments, the side chain of X¹³ is C₁₋₆ alkyl. In some embodiments, X¹³ is I. In some embodiments, X¹³ is L. In some embodiments, X¹³ is V. In some embodiments, X¹³ is M.

Various amino acid residues may be utilized for X¹⁴. For example, in some embodiments, the side chain of X¹⁴ is C₁₋₆ aliphatic. In some embodiments, the side chain of X¹⁴ is C₁₋₆ alkyl. In some embodiments, X¹⁴ is V. In some embodiments, X¹⁴ is L. In some embodiments, X¹⁴ is H.

In some embodiments, an agent described herein, e.g., a stapled peptide, interacts with one or more (e.g., 1, 2, 3, 4, 5, 6, 7, 8, 9, 10, 11, 12, 13, or 14) or all of the following amino acid residues of WWP1 or amino acid residues corresponding thereto: Glu702, Phe703, Ser706, Leu707, Trp709, Ile710, Glu717, Cys718, Gly719, Leu720, Glu721, Met722, Val726, and Met760. In some embodiments, it interacts with Glu702 of WWP1 or an amino acid residue corresponding thereto. In some embodiments, it interacts with Phe703 of WWP1 or an amino acid residue corresponding thereto. In some embodiments, it interacts with Ser706 of WWP1 or an amino acid residue corresponding thereto. In some embodiments, it interacts with Leu707 of WWP1 or an amino acid residue corresponding thereto. In some embodiments, it interacts with Trp709 of WWP1 or an amino acid residue corresponding thereto. In some embodiments, it interacts with Ile710 of WWP1 or an amino acid residue corresponding thereto. In some embodiments, it interacts with Glu717 of WWP1 or an amino acid residue corresponding thereto. In some embodiments, it interacts with Cys718 of WWP1 or an amino acid residue corresponding thereto. In some embodiments, it interacts with Gly719 of WWP1 or an amino acid residue corresponding thereto. In some embodiments, it interacts with Leu720 of WWP1 or an amino acid residue corresponding thereto. In some embodiments, it interacts with Glu721 of WWP1 or an amino acid residue corresponding thereto. In some embodiments, it interacts with Met722 of WWP1 or an amino acid residue corresponding thereto. In some embodiments, it interacts with Val726 of WWP1 or an amino acid residue corresponding thereto. In some embodiments, it interacts with Met760 of WWP1 or an amino acid residue corresponding thereto.

In some embodiments, an agent described herein, e.g., a stapled peptide, interacts with one or more (e.g., 1, 2, 3, 4, 5, 6, 7, 8, 9, 10, 11, 12, 13, 14, 15, 16, 17, or 18) or all of the following amino acid residues of WWP2 or amino acid residues corresponding thereto: Glu650, Phe651, Asn653, Ser654, Ile655, Trp657, Ile658, Asn661, Asn662, Glu665, Cys666, Gly667, Leu668, Glu669, Leu670, Gln674, Tyr704, and Leu708. In some embodiments, it interacts with Glu650 of WWP2 or an amino acid residue corresponding thereto. In some embodiments, it interacts with Phe651 of WWP2 or an amino acid residue corresponding thereto. In some embodiments, it interacts with Asn653 of WWP2 or an amino acid residue corresponding thereto. In some embodiments, it interacts with Ser654 of WWP2 or an amino acid residue corresponding thereto. In some embodiments, it interacts with Ile655 of WWP2 or an amino acid residue corresponding thereto. In some embodiments, it interacts with Trp657 of WWP2 or an amino acid residue corresponding thereto. In some embodiments, it interacts with Ile658 of WWP2 or an amino acid residue corresponding thereto. In some embodiments, it interacts with Asn661 of WWP2 or an amino acid residue corresponding thereto. In some embodiments, it interacts with Asn662 of WWP2 or an amino acid residue corresponding thereto. In some embodiments, it interacts with Glu665 of WWP2 or an amino acid residue corresponding thereto. In some embodiments, it interacts with Cys666 of WWP2 or an amino acid residue corresponding thereto. In some embodiments, it interacts with Gly667 of WWP2 or an amino acid residue corresponding thereto. In some embodiments, it interacts with Leu668 of WWP2 or an amino acid residue corresponding thereto. In some embodiments, it interacts with Glu669 of WWP2 or an amino acid residue corresponding thereto. In some embodiments, it interacts with Leu670 of WWP2 or an amino acid residue corresponding thereto. In some embodiments, it interacts with Gln674 of WWP2 or an amino acid residue corresponding thereto. In some embodiments, it interacts with Tyr704 of WWP2 or an amino acid residue corresponding thereto. In some embodiments, it interacts with Leu708 of WWP2 or an amino acid residue corresponding thereto.

In some embodiments, an agent comprising a cluster C71 sequence is capable of binding to WWP1. In some embodiments, an agent comprising a cluster C71 sequence is capable of binding to an autoinhibited form of WWP1. In some embodiments, an agent comprising a cluster C71 sequence is capable of binding to WWP2.

In some embodiments, a cluster comprising a cluster C71 sequence is capable of selectively binding to an active form of WWP1 over an autoinhibited form thereof. In some embodiments, a cluster comprising a cluster C71 sequence is capable of selectively binding to an active form of WWP2 over an active form of WWP1.

### Cluster C72

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C72 as described herein. In some embodiments, an agent comprises X⁷²X²X³X⁴X⁵X⁶X⁷X⁸X⁹X¹⁰X¹¹X¹²X¹³X¹⁴, wherein each of X⁷², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷²X²X³X⁴X⁵X⁶X⁷X⁸X⁹X¹⁰X¹¹X¹²X¹³X¹⁴,

wherein:
each of X⁷², X², X³, X⁴, X⁵, X⁶, X⁷, X⁸, X⁹, X¹⁰, X¹¹, X¹², X¹³, and X¹⁴ is independently an amino acid residue;
X⁹ comprises a side chain comprising a polar group;
X¹³ comprises a hydrophobic side chain or a side chain comprising an aromatic group; and
X¹⁴ comprises a hydrophobic side chain or a side chain comprising an aromatic group.

Various amino acid residues may be utilized for X⁷². For example, in some embodiments, the side chain of X⁷² is hydrophobic. In some embodiments, a cluster is enriched for amino acid residues comprising a hydrophobic side chain at X⁷². In some embodiments, the side chain of X⁷² is aliphatic. In some embodiments, the side chain of X⁷² is C₁₋₆ alkyl. In some embodiments, X⁷² is A. In some embodiments, X⁷² is I. In some embodiments, X⁷² is L.

Various amino acid residues may be utilized for X². For example, in some embodiments, X² comprises a side chain comprising a polar group. In some embodiments, a cluster is enriched for amino acid residues whose side chains comprise a polar group at X². In some embodiments, X² comprises a side chain comprising —C(O)NH₂. In some embodiments, X² is N. In some embodiments, X² is Q. In some embodiments, the side chain of X² is hydrophobic. In some embodiments, a cluster is enriched for amino acid residues comprising a hydrophobic side chain at X². In some embodiments, the side chain of X² is aliphatic. In some embodiments, the side chain of X² is C₁₋₆ alkyl. In some embodiments, X² is V. In some embodiments, X² is I. In some embodiments, X² is L. In some embodiments, X² comprises a side chain comprising an aromatic group. In some embodiments, X² is H. In some embodiments, X² is Y. In some embodiments, X² is W.

Various amino acid residues may be utilized for X³. For example, in some embodiments, X³ comprises a side chain comprising an aromatic group. In some embodiments, X³ is H. In some embodiments, X³ is F. In some embodiments, X³ is Y. In some embodiments, X³ is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

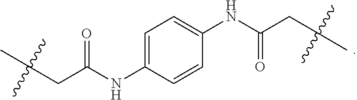 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues whose side chains comprise a basic group at X⁵. In some embodiments, X⁵ is R. In some embodiments, X⁵ is H. In some embodiments, the side chain of X⁵ is hydrophobic. In some embodiments, a cluster is enriched for amino acid residues comprising a hydrophobic side chain at X⁵. In some embodiments, the side chain of X⁵ is aliphatic. In some embodiments, the side chain of X⁵ is C₁₋₆ alkyl. In some embodiments, X⁵ is L.

Various amino acid residues may be utilized for X⁶. For example, in some embodiments, X⁶ comprises a hydrophobic side chain. In some embodiments, a cluster is enriched for amino acid residues comprising a hydrophobic side chain at X⁶. In some embodiments, the side chain of X⁶ is C₁₋₆ aliphatic. In some embodiments, the side chain of X⁶ is C₁₋₆ alkyl. In some embodiments, X⁶ is A. In some embodiments, X⁶ is V.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising an acidic group. In some embodiments, a cluster is enriched for amino acid residues whose side chains comprise an acidic group at X⁹. In some embodiments, X⁹ comprises a side chain comprising —COOH. In some embodiments, X⁹ is D. In some embodiments, X⁹ is E.

Various amino acid residues may be utilized for X¹⁰. For example, in some embodiments, X¹⁰ comprises a side chain comprising a polar group. In some embodiments, a cluster is enriched for amino acid residues whose side chains comprise polar groups at X¹⁰. In some embodiments, X¹⁰ comprises a side chain comprising —OH. In some embodiments, X¹⁰ is T. In some embodiments, the side chain of X¹⁰ is hydrophobic. In some embodiments, a cluster is enriched for amino acid residues comprising a hydrophobic side chain at X¹⁰. In some embodiments, X¹⁰ is M. In some embodiments, the side chain of X¹⁰ is aliphatic. In some embodiments, the side chain of X¹⁰ is C₁₋₆ alkyl. In some embodiments, X¹⁰ is V. In some embodiments, X¹⁰ is L.

Various amino acid residues may be utilized for X¹³. In some embodiments, X¹³ comprises a side chain comprising an aromatic group. In some embodiments, X¹³ is F.

Various amino acid residues may be utilized for X¹⁴. In some embodiments, X¹⁴ comprises a side chain comprising an aromatic group. In some embodiments, X¹⁴ is F. In some embodiments, X¹⁴ is W.

In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of WWP2 or amino acid residues corresponding thereto: Tyr499, Phe495, Arg496, His500, Arg503, Phe504, His507, Ser508, Gly619, Lys620, Phe621, Leu747, Met748, Met752, and Glu789. In some embodiments, it interacts with Tyr499 of WWP2 or an amino acid residue corresponding thereto. In some embodiments, it interacts with Phe495 of WWP2 or an amino acid residue corresponding thereto. In some embodiments, it interacts with Arg496 of WWP2 or an amino acid residue corresponding thereto. In some embodiments, it interacts with His500 of WWP2 or an amino acid residue corresponding thereto. In some embodiments, it interacts with Arg503 of WWP2 or an amino acid residue corresponding thereto. In some embodiments, it interacts with Phe504 of WWP2 or an amino acid residue corresponding thereto. In some embodiments, it interacts with His507 of WWP2 or an amino acid residue corresponding thereto. In some embodiments, it interacts with Ser508 of WWP2 or an amino acid residue corresponding thereto. In some embodiments, it interacts with Gly619 of WWP2 or an amino acid residue corresponding thereto. In some embodiments, it interacts with Lys620 of WWP2 or an amino acid residue corresponding thereto. In some embodiments, it interacts with Phe621 of WWP2 or an amino acid residue corresponding thereto. In some embodiments, it interacts with Leu747 of WWP2 or an amino acid residue corresponding thereto. In some embodiments, it interacts with Met748 of WWP2 or an amino acid residue corresponding thereto. In some embodiments, it interacts with Met752 of WWP2 or an amino acid residue corresponding thereto. In some embodiments, it interacts with Glu789 of WWP2 or an amino acid residue corresponding thereto.

In some embodiments, an agent comprising a cluster C72 sequence is capable of binding to WWP2. In some embodiments, an agent comprising a cluster C72 sequence is capable of selectively binding to WWP2 over WWP1.

### Cluster C73

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C73 as described herein. In some embodiments, an agent comprises X⁷³X²X³X⁴X⁵X⁶X⁷X⁸X⁹X¹⁰X¹¹X¹²X¹³X¹⁴, wherein each of X⁷³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷³X²X³X⁴X⁵X⁶X⁷X⁸X⁹X¹⁰X¹¹X¹²X¹³X¹⁴,

wherein:
each of X⁴, X⁵, X⁶, X⁷, X⁸, X⁹, X¹⁰, X¹¹, X¹², X¹³, and X¹⁴ is independently an amino acid residue; and
X² comprises a hydrophobic side chain or a side chain comprising an aromatic group.

Various amino acid residues may be utilized for X⁷³. For example, in some embodiments, X⁷³ comprises a side chain comprising a polar group. In some embodiments, a cluster is enriched for amino acid residues whose side chains comprise a polar group at X⁷³. In some embodiments, X⁷³ comprises a side chain comprising —OH. In some embodiments, X⁷³ is T. In some embodiments, the side chain of X⁷³ is hydrophobic. In some embodiments, a cluster is enriched for amino acid residues comprising a hydrophobic side chain at X⁷³. In some embodiments, the side chain of X⁷³ is aliphatic. In some embodiments, the side chain of X⁷³ is C₁₋₆ alkyl. In some embodiments, X⁷³ is A. In some embodiments, X⁷³ is V. In some embodiments, X⁷³ is I. In some embodiments, X⁷³ is L.

Various amino acid residues may be utilized for X². For example, in some embodiments, X² comprises a side chain comprising an aromatic group. In some embodiments, X² is W.

Various amino acid residues may be utilized for X³. For example, in some embodiments, X³ comprises a side chain comprising an acidic group. In some embodiments, a cluster is enriched for amino acid residues whose side chains comprise an acidic group at X³. In some embodiments, X³ comprises a side chain comprising —COOH. In some embodiments, X³ is D. In some embodiments, X³ is E.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

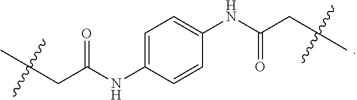 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a basic group. In some embodiments, a cluster is enriched for amino acid residues whose side chains comprise a basic group at X⁵. In some embodiments, X⁵ is R.

Various amino acid residues may be utilized for X⁶. For example, in some embodiments, X⁶ comprises a side chain comprising a polar group. In some embodiments, a cluster is enriched for amino acid residues whose side chains comprise a polar group at X⁶. In some embodiments, X⁶ comprises a side chain comprising —C(O)NH2. In some embodiments, X⁶ is N. In some embodiments, X⁶ is Q. In some embodiments, X⁶ comprises a side chain comprising an aromatic group. In some embodiments, X⁶ is Y. In some embodiments, X⁶ is W.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising an aromatic group. In some embodiments, X⁹ is H. In some embodiments, X⁹ is F. In some embodiments, X⁹ is W.

Various amino acid residues may be utilized for X¹⁰. For example, in some embodiments, X¹⁰ comprises a side chain comprising an acidic group. In some embodiments, a cluster is enriched for amino acid residues whose side chains comprise an acidic group at X¹⁰. In some embodiments, X¹⁰ comprises a side chain comprising —COOH. In some embodiments, X¹⁰ is D. In some embodiments, X¹⁰ is E. In some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —C(O)NH₂. In some embodiments, X¹⁰ is N. In some embodiments, X¹⁰ is Q.

Various amino acid residues may be utilized for X¹². For example, in some embodiments, X¹² comprises a side chain comprising an aromatic group. In some embodiments, X¹² is F. In some embodiments, X¹² is Y. In some embodiments, X¹² is W.

Various amino acid residues may be utilized for X¹³. For example, in some embodiments, the side chain of X¹³ is hydrophobic. In some embodiments, a cluster is enriched for amino acid residues comprising a hydrophobic side chain at X¹³. In some embodiments, X¹³ is M. In some embodiments, the side chain of X¹³ is aliphatic. In some embodiments, the side chain of X¹³ is C₁₋₆ alkyl. In some embodiments, X¹³ is V. In some embodiments, X¹³ is I. In some embodiments, X¹³ is L.

Various amino acid residues may be utilized for X¹⁴. For example, in some embodiments, X¹⁴ comprises a side chain comprising a polar group. In some embodiments, a cluster is enriched for amino acid residues whose side chains comprise a polar group at X¹⁴. In some embodiments, X¹⁴ comprises a side chain comprising —C(O)NH₂. In some embodiments, X¹⁴ is N. In some embodiments, X¹⁴ is Q.

In some embodiments, an agent described herein, e.g., a stapled peptide, interacts with one or more (e.g., 1, 2, 3, 4, 5, 6, 7, 8, 9, 10, 11, 12, 13, 14, 15, 16, 17, 18, 19, 20, 21, or 22) or all of the following amino acid residues of WWP2 or amino acid residues corresponding thereto: Phe495, Arg496, Tyr499, His500, Arg561, Glu562, Phe565, Leu566, Gly619, Lys620, Phe621, Ile622, Asp623, Leu747, Cys750, Gly751, Met752, Gln753, Glu754, Asp787, Glu789, and Arg803. In some embodiments, it interacts with Phe495 of WWP2 or an amino acid residue corresponding thereto. In some embodiments, it interacts with Arg496 of WWP2 or an amino acid residue corresponding thereto. In some embodiments, it interacts with Tyr499 of WWP2 or an amino acid residue corresponding thereto. In some embodiments, it interacts with His500 of WWP2 or an amino acid residue corresponding thereto. In some embodiments, it interacts with Arg561 of WWP2 or an amino acid residue corresponding thereto. In some embodiments, it interacts with Glu562 of WWP2 or an amino acid residue corresponding thereto. In some embodiments, it interacts with Phe565 of WWP2 or an amino acid residue corresponding thereto. In some embodiments, it interacts with Leu566 of WWP2 or an amino acid residue corresponding thereto. In some embodiments, it interacts with Gly619 of WWP2 or an amino acid residue corresponding thereto. In some embodiments, it interacts with Lys620 of WWP2 or an amino acid residue corresponding thereto. In some embodiments, it interacts with Phe621 of WWP2 or an amino acid residue corresponding thereto. In some embodiments, it interacts with Ile622 of WWP2 or an amino acid residue corresponding thereto. In some embodiments, it interacts with Asp623 of WWP2 or an amino acid residue corresponding thereto. In some embodiments, it interacts with Leu747 of WWP2 or an amino acid residue corresponding thereto. In some embodiments, it interacts with Cys750 of WWP2 or an amino acid residue corresponding thereto. In some embodiments, it interacts with Gly751 of WWP2 or an amino acid residue corresponding thereto. In some embodiments, it interacts with Met752 of WWP2 or an amino acid residue corresponding thereto. In some embodiments, it interacts with Gln753 of WWP2 or an amino acid residue corresponding thereto. In some embodiments, it interacts with Glu754 of WWP2 or an amino acid residue corresponding thereto. In some embodiments, it interacts with Asp787 of WWP2 or an amino acid residue corresponding thereto. In some embodiments, it interacts with Glu789 of WWP2 or an amino acid residue corresponding thereto. In some embodiments, it interacts with Arg803 of WWP2 or an amino acid residue corresponding thereto.

In some embodiments, an agent comprising a cluster C73 sequence is capable of binding to WWP2. In some embodiments, an agent comprising a cluster C73 sequence is capable of binding to WWP1. In some embodiments, an agent comprising a cluster C73 sequence is capable of selectively binding to an active WWP1 over an autoinhibited form of WWP1.

### Cluster C74

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C74 as described herein. In some embodiments, an agent comprises X⁷⁴X²X³X⁴X⁵X⁶X⁷X⁸X⁹X¹⁰X¹¹X¹²X¹³X¹⁴, wherein each of X⁷⁴, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷⁴X²X³X⁴X⁵X⁶X⁷X⁸X⁹X¹⁰X¹¹X¹²X¹³X¹⁴,

wherein:
each of X³⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain; and
X¹² comprises a side chain comprising a basic group or an aromatic group.

Various amino acid residues may be utilized for X². For example, in some embodiments, X² comprises a hydrophobic side chain. In some embodiments, a cluster is enriched for amino acid residues comprising a hydrophobic side chain at X². In some embodiments, X² is M. In some embodiments, the side chain of X² is aliphatic. In some embodiments, the side chain of X² is C₁₋₆ alkyl. In some embodiments, X² is V. In some embodiments, X² is I. In some embodiments, X² is L.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

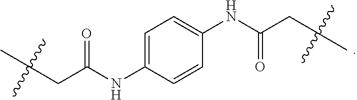 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a polar group. In some embodiments, a cluster is enriched for amino acid residues whose side chains comprise a polar group at X⁵. In some embodiments, X⁵ comprises a side chain comprising —OH. In some embodiments, X⁵ is S. In some embodiments, X⁵ is T. In some embodiments, the side chain of X⁵ is hydrophobic. In some embodiments, a cluster is enriched for amino acid residues comprising a hydrophobic side chain at X⁵. In some embodiments, the side chain of X⁵ is aliphatic. In some embodiments, the side chain of X⁵ is C₁₋₆ alkyl. In some embodiments, X⁵ is A. In some embodiments, X⁵ is V.

Various amino acid residues may be utilized for X⁶. For example, in some embodiments, the side chain of X⁶ is hydrophobic. In some embodiments, a cluster is enriched for amino acid residues comprising a hydrophobic side chain at X⁶. In some embodiments, X⁶ is M. In some embodiments, the side chain of X⁶ is aliphatic. In some embodiments, the side chain of X⁶ is C₁₋₆ alkyl. In some embodiments, X⁶ is V. In some embodiments, X⁶ is L. In some embodiments, X⁶ comprises a side chain comprising an aromatic group. In some embodiments, X⁶ is H. In some embodiments, X⁶ is F. In some embodiments, X⁶ is W.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising a polar group. In some embodiments, a cluster is enriched for amino acid residues whose side chains comprise a polar group at X⁹. In some embodiments, X⁹ comprises a side chain comprising —C(O)NH₂. In some embodiments, X⁹ is N. In some embodiments, X⁹ is Q. In some embodiments, the side chain of X⁹ is hydrophobic. In some embodiments, a cluster is enriched for amino acid residues comprising a hydrophobic side chain at X⁹. In some embodiments, the side chain of X⁹ is aliphatic. In some embodiments, the side chain of X⁹ is C₁₋₆ alkyl. In some embodiments, X⁹ is V.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, X¹⁰ is M. In some embodiments, the side chain of X¹⁰ is C₁₋₆ aliphatic. In some embodiments, the side chain of X¹⁰ is C₁₋₆ alkyl. In some embodiments, X¹⁰ is L. In some embodiments, X¹⁰ is I. In some embodiments, X¹⁰ is V.

Various amino acid residues may be utilized for X¹². For example, in some embodiments, X¹² comprises a side chain comprising a basic group. In some embodiments, a cluster is enriched for amino acid residues whose side chains comprise a basic group at X¹². In some embodiments, X¹² comprises a side chain comprising an aromatic group. In some embodiments, X¹² is H.

Various amino acid residues may be utilized for X¹³. For example, in some embodiments, X¹³ comprises a side chain comprising a polar group. In some embodiments, a cluster is enriched for amino acid residues whose side chains comprise a polar group at X¹³. In some embodiments, X¹³ comprises a side chain comprising —OH. In some embodiments, X¹³ is S. In some embodiments, X¹³ is T. In some embodiments, X¹³ comprises a side chain comprising —C(O)NH₂. In some embodiments, X¹³ is N. In some embodiments, X¹³ is Q.

Various amino acid residues may be utilized for X¹⁴. For example, in some embodiments, X¹⁴ comprises a side chain comprising an acidic group. In some embodiments, a cluster is enriched for amino acid residues whose side chains comprise an acidic group at X¹⁴. In some embodiments, X¹⁴ comprises a side chain comprising —COOH. In some embodiments, X¹⁴ is D. In some embodiments, X¹⁴ comprises a side chain comprising a polar group. In some embodiments, a cluster is enriched for amino acid residues whose side chains comprise a polar group at X¹⁴. In some embodiments, X¹⁴ comprises a side chain comprising —OH. In some embodiments, X¹⁴ is S. In some embodiments, X¹⁴ is T.

In some embodiments, an agent comprising a cluster C74 sequence is capable of selectively binding to an autoinhibited form of WWP1 over an active form thereof. In some embodiments, an agent comprising a cluster C74 sequence is capable of selectively binding to an autoinhibited form of WWP1 over WWP2.

### Cullin-RING Family

In some embodiments, the present disclosure provides agents that can bind to Cullin-Ring E3 ligases. In some embodiments, the present disclosure provides methods for modulating a function or interaction of such an E3 ligase, comprising contacting the ligase with a provided agent.

### Cluster C75

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C75 as described herein. In some embodiments, an agent comprises X⁷⁵X²X³X⁴X⁵X⁶X⁷X⁸X⁹X¹⁰X¹¹X¹²X¹³X¹⁴, wherein each of X⁷⁵, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷⁵X²X³X⁴X⁵X⁶X⁷X⁸X⁹X¹⁰X¹¹X¹²X¹³X¹⁴,

wherein:
each of X⁷⁵, X², X³, X⁴, X⁵, X⁶, X⁷, X⁸, X⁹, X¹⁰, X¹¹, X¹², X¹³, and X¹⁴ is independently an amino acid residue;
X⁷⁵ comprises a hydrophobic side chain or a side chain comprising an aromatic group; and
X¹² comprises a side chain comprising an acidic group.

Various amino acid residues may be utilized for X³⁵. In some embodiments, X³s is W.

Various amino acid residues may be utilized for X². For example, in some embodiments, X² comprises a hydrophobic side chain. In some embodiments, a cluster is enriched for amino acid residues comprising a hydrophobic side chain at X². In some embodiments, X² comprises a side chain comprising an aromatic group. In some embodiments, X² is H. In some embodiments, X² is Y. In some embodiments, X² is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

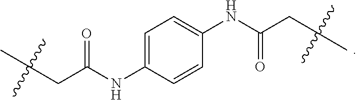 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, the side chain of X⁵ is hydrophobic. In some embodiments, a cluster is enriched for amino acid residues comprising a hydrophobic side chain at X⁵. In some embodiments, X⁵ is M. In some embodiments, the side chain of X⁵ is aliphatic. In some embodiments, the side chain of X⁵ is C₁₋₆ alkyl. In some embodiments, X⁵ is V. In some embodiments, X⁵ is I. In some embodiments, X⁵ is L. For example, in some embodiments, the side chain of X⁵ comprises an aromatic group. In some embodiments, a cluster is enriched for amino acid residues comprising an aromatic group at X⁵. In some embodiments, the side chain of X⁵ comprises a polar group. In some embodiments, a cluster is enriched for amino acid residues comprising a polar group at X⁵. In some embodiments, X⁵ comprises a side chain comprising —OH. In some embodiments, X⁵ is Y. In some embodiments, X⁵ is F.

Various amino acid residues may be utilized for X⁶. For example, in some embodiments, X⁶ comprises a side chain comprising an acidic group. In some embodiments, a cluster is enriched for amino acid residues whose side chains comprise acidic groups at X⁶. In some embodiments, X⁶ comprises a side chain comprising —COOH. In some embodiments, X⁶ is D.

Various amino acid residues may be utilized for X⁷. In some embodiments, X⁷ is A.

Various amino acid residues may be utilized for X⁸. In some embodiments, X⁸ is A.

Various amino acid residues may be utilized for X¹². For example, in some embodiments, X¹² comprises a side chain comprising an acidic group. In some embodiments, a cluster is enriched for amino acid residues whose side chains comprise an acidic group at X¹². In some embodiments, X¹² comprises a side chain comprising —COOH. In some embodiments, X¹² is D.

Various amino acid residues may be utilized for X¹³. For example, in some embodiments, the side chain of X¹³ comprises an aromatic group. In some embodiments, a cluster is enriched for amino acid residues comprising an aromatic group at X¹³. In some embodiments, the side chain of X¹³ comprises a polar group. In some embodiments, a cluster is enriched for amino acid residues comprising a polar group at X¹³. In some embodiments, X¹³ comprises a side chain comprising —OH. In some embodiments, X¹³ is Y. In some embodiments, X¹³ is N.

Various amino acid residues may be utilized for X¹⁴. For example, in some embodiments, X¹⁴ comprises a hydrophobic side chain. In some embodiments, a cluster is enriched for amino acid residues comprising a hydrophobic side chain at X¹⁴. In some embodiments, the side chain of X¹⁴ is C₁₋₆ aliphatic. In some embodiments, the side chain of X¹⁴ is C₁₋₆ alkyl. In some embodiments, the side chain of X¹⁴ comprises an aromatic group. In some embodiments, X¹⁴ is F. In some embodiments, X¹⁴ is M. In some embodiments, X¹⁴ is I. In some embodiments, X¹⁴ is V. In some embodiments, X¹⁴ is Y. In some embodiments, X¹⁴ is Q.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of VHL or amino acid residues corresponding thereto: Pro59, Arg60, Val62, Leu63, Arg64, Val66, Gly114, His115, Leu116, Thr133, Glu134, Leu135, Val137, Leu201, Thr202, Arg205, and Ile206. In some embodiments, it interacts with Pro59 of VHL or an amino acid residue corresponding thereto. In some embodiments, it interacts with Arg60 of VHL or an amino acid residue corresponding thereto. In some embodiments, it interacts with Val62 of VHL or an amino acid residue corresponding thereto. In some embodiments, it interacts with Leu63 of VHL or an amino acid residue corresponding thereto. In some embodiments, it interacts with Arg64 of VHL or an amino acid residue corresponding thereto. In some embodiments, it interacts with Val66 of VHL or an amino acid residue corresponding thereto. In some embodiments, it interacts with Gly114 of VHL or an amino acid residue corresponding thereto. In some embodiments, it interacts with His115 of VHL or an amino acid residue corresponding thereto. In some embodiments, it interacts with Leu116 of VHL or an amino acid residue corresponding thereto. In some embodiments, it interacts with Thr133 of VHL or an amino acid residue corresponding thereto. In some embodiments, it interacts with Glu134 of VHL or an amino acid residue corresponding thereto. In some embodiments, it interacts with Leu135 of VHL or an amino acid residue corresponding thereto. In some embodiments, it interacts with Val137 of VHL or an amino acid residue corresponding thereto. In some embodiments, it interacts with Leu201 of VHL or an amino acid residue corresponding thereto. In some embodiments, it interacts with Thr202 of VHL or an amino acid residue corresponding thereto. In some embodiments, it interacts with Arg205 of VHL or an amino acid residue corresponding thereto. In some embodiments, it interacts with Ile206 of VHL or an amino acid residue corresponding thereto.

In some embodiments, an agent comprising a cluster C73 sequence is capable of binding to VHL.

### Cluster C76

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C76 as described herein. In some embodiments, an agent comprises X⁷⁶X²X³X⁴X⁵X⁶X⁷X⁸X⁹X¹⁰X¹¹X¹²X¹³X¹⁴, wherein each of X⁷⁶, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷⁶X²X³X⁴X⁵X⁶X⁷X⁸X⁹X¹⁰X¹¹X¹²X¹³X¹⁴,

wherein:
each of X⁷⁶, X², X³, X⁴, X⁵, X⁶, X⁷, X⁸, X⁹, X¹⁰, X¹¹, X¹², X¹³, and X¹⁴ is independently an amino acid residue;
X⁷⁶ comprises a hydrophobic side chain or a side chain comprising an aromatic group; and
X¹² comprises a side chain comprising an acidic group.

Various amino acid residues may be utilized for X⁷⁶. In some embodiments, X⁷⁶ is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

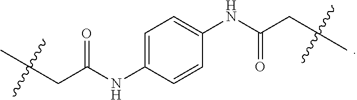 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, the side chain of X⁵ is hydrophobic. In some embodiments, a cluster is enriched for amino acid residues comprising a hydrophobic side chain at X⁵. In some embodiments, the side chain of X⁵ is aliphatic. In some embodiments, the side chain of X⁵ is C₁₋₆ alkyl. In some embodiments, X⁵ is A.

Various amino acid residues may be utilized for X⁶. For example, in some embodiments, X⁶ comprises a side chain comprising an acidic group. In some embodiments, a cluster is enriched for amino acid residues whose side chains comprise an acidic group at X⁶. In some embodiments, X⁶ comprises a side chain comprising —COOH. In some embodiments, X⁶ is E.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a hydrophobic side chain. In some embodiments, X⁹ comprises a side chain comprising an aromatic group. In some embodiments, X⁹ is W.

In some embodiments, an agent described herein, e.g., a stapled peptide, interacts with one or more (e.g., 1, 2, 3, 4, 5, 6, 7, 8, 9, or 10) or all of the following amino acid residues of CUL5 or amino acid residues corresponding thereto: Val35, Thr36, Lys37, Trp40, Phe41, Phe44, His48, Ile106, Lys109, Cys112, and Gln113. In some embodiments, it interacts with Val35 of CUL5 or an amino acid residue corresponding thereto. In some embodiments, it interacts with Thr36 of CUL5 or an amino acid residue corresponding thereto. In some embodiments, it interacts with Lys37 of CUL5 or an amino acid residue corresponding thereto. In some embodiments, it interacts with Trp40 of CUL5 or an amino acid residue corresponding thereto. In some embodiments, it interacts with Phe41 of CUL5 or an amino acid residue corresponding thereto. In some embodiments, it interacts with Phe44 of CUL5 or an amino acid residue corresponding thereto. In some embodiments, it interacts with His48 of CUL5 or an amino acid residue corresponding thereto. In some embodiments, it interacts with Ile106 of CUL5 or an amino acid residue corresponding thereto. In some embodiments, it interacts with Lys109 of CUL5 or an amino acid residue corresponding thereto. In some embodiments, it interacts with Cys112 of CUL5 or an amino acid residue corresponding thereto. In some embodiments, it interacts with Gln113 of CUL5 or an amino acid residue corresponding thereto.

In some embodiments, an agent comprising a cluster C76 sequence is capable of binding to CUL5.

### Cluster C77

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C77 as described herein. In some embodiments, an agent comprises X⁷⁷X²X³X⁴X⁵X⁶X⁷X⁸X⁹X¹⁰X¹¹X¹²X¹³X¹⁴, wherein each of X⁷⁷, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷⁷X²X³X⁴X⁵X⁶X⁷X⁸X⁹X¹⁰X¹¹X¹²X¹³X¹⁴,

wherein:
each of X⁷⁷, X², X³, X⁴, X⁵, X⁶, X⁷, X⁸, X⁹, X¹⁰, X¹¹, X¹², X¹³, and X¹⁴ is independently an amino acid residue;
X⁶ comprises a hydrophobic side chain; and
X⁹ comprises a hydrophobic side chain or a side chain comprising an aromatic group.

Various amino acid residues may be utilized for X⁷⁷. For example, in some embodiments, X⁷⁷ comprises a side chain comprising an acidic group. In some embodiments, a cluster is enriched for amino acid residues whose side chains comprise an acidic group at X⁷⁷. In some embodiments, X⁷⁷ comprises a side chain comprising —COOH. In some embodiments, X⁷⁷ is D. In some embodiments, X⁷⁷ is E. In some embodiments, X⁷⁷ comprises a side chain comprising a polar group. In some embodiments, a cluster is enriched for amino acid residues whose side chains comprise a polar group at X⁷⁷. In some embodiments, X⁷⁷ comprises a side chain comprising —OH. In some embodiments, X⁷⁷ is S. In some embodiments, X⁷⁷ is T.

Various amino acid residues may be utilized for X². In some embodiments, wherein X² comprises a side chain comprising a basic group. In some embodiments, X² is R.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

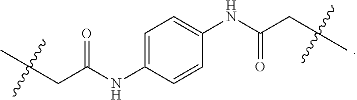 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprise an acidic group at X⁵. In some embodiments, X⁵ comprises a side chain comprising —COOH. In some embodiments, X⁵ is D. In some embodiments, X⁵ is E.

Various amino acid residues may be utilized for X⁶. For example, in some embodiments, X⁶ is M. In some embodiments, the side chain of X⁶ is aliphatic. In some embodiments, the side chain of X⁶ is C₁₋₆ alkyl. In some embodiments, X⁶ is L. In some embodiments, X⁶ comprises a side chain comprising an aromatic group. In some embodiments, X⁶ is F.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising an aromatic group. In some embodiments, X⁹ is W.

Various amino acid residues may be utilized for X¹⁰. In some embodiments, X¹⁰ comprises a hydrophobic side chain. In some embodiments, the side chain of X¹⁰ is aliphatic. In some embodiments, the side chain of X¹⁰ is C₁₋₆ alkyl. In some embodiments, X¹⁰ is A. In some embodiments, X¹⁰ is V. In some embodiments, X¹⁰ is I.

Various amino acid residues may be utilized for X¹². For example, in some embodiments, the side chain of X¹² is hydrophobic. In some embodiments, a cluster is enriched for amino acid residues comprising a hydrophobic side chain at X¹². In some embodiments, the side chain of X¹² is aliphatic. In some embodiments, the side chain of X¹² is C₁₋₆ alkyl. In some embodiments, X¹² is A. In some embodiments, X¹² is V. In some embodiments, X¹² is I. In some embodiments, X¹² comprises a side chain comprising an acidic group. In some embodiments, a cluster is enriched for amino acid residues whose side chains comprise an acidic group at X¹². In some embodiments, X¹² comprises a side chain comprising —COOH. In some embodiments, X¹² is D. In some embodiments, X¹² is E.

Various amino acid residues may be utilized for X¹⁴. For example, in some embodiments, the side chain of X¹⁴ is hydrophobic. In some embodiments, a cluster is enriched for amino acid residues comprising a hydrophobic side chain at X¹⁴. In some embodiments, X¹⁴ comprises a side chain comprising an aromatic group. In some embodiments, X¹⁴ comprises a side chain comprising —OH. In some embodiments, X¹⁴ is F. In some embodiments, X¹⁴ is Y. In some embodiments, X¹⁴ is W. In some embodiments, X¹⁴ is T.

In some embodiments, an agent comprising a cluster C77 sequence is capable of binding to CUL4B.

### Cluster C78

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C78 as described herein. In some embodiments, an agent comprises X⁷⁸X²X³X⁴X⁵X⁶X⁷X⁸X⁹X¹⁰X¹¹X¹²X¹³X¹⁴, wherein each of X⁷⁸, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising X⁷⁸X²X³X⁴X⁵X⁶X⁷X⁸X⁹X¹⁰X¹¹X¹²X¹³X¹⁴,

wherein:
each of X⁷⁸, X², X³, X⁴, X⁵, X⁶, X⁷, X⁸, X⁹, X¹⁰, X¹¹, X¹², X¹³, and X¹⁴ is independently an amino acid residue;
X⁵ comprises a side chain comprising an acidic group; and
X¹³ comprises an aliphatic side chain.

Various amino acid residues may be utilized for X⁷⁸. For example, in some embodiments, X⁷⁸ comprises a side chain comprising an acidic group. In some embodiments, a cluster is enriched for amino acid residues whose side chains comprise an acidic group at X⁷⁸. In some embodiments, X⁷⁸ comprises a side chain comprising —COOH. In some embodiments, X⁷⁸ is E.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

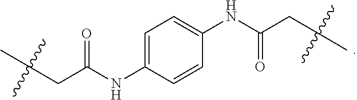 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising —COOH. In some embodiments, X⁵ is D. In some embodiments, X⁵ is E.

Various amino acid residues may be utilized for X⁶. For example, in some embodiments, the side chain of X⁶ is hydrophobic. In some embodiments, a cluster is enriched for amino acid residues comprising a hydrophobic side chain at X⁶. In some embodiments, X⁶ is M. In some embodiments, the side chain of X⁶ is aliphatic. In some embodiments, the side chain of X⁶ is C₁₋₆ alkyl. In some embodiments, X⁶ is V. In some embodiments, X⁶ is I. In some embodiments, X⁶ is L. In some embodiments, X⁶ comprises a side chain comprising an aromatic group. In some embodiments, X⁶ is H. In some embodiments, X⁶ is Y. In some embodiments, X⁶ is W.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ is hydrophobic. In some embodiments, a cluster is enriched for amino acid residues comprising a hydrophobic side chain at X⁹. In some embodiments, the side chain of X⁹ is aliphatic. In some embodiments, the side chain of X⁹ is C₁₋₆ alkyl. In some embodiments, X⁹ is A.

Various amino acid residues may be utilized for X¹⁰. For example, in some embodiments, the side chain of X¹⁰ is hydrophobic. In some embodiments, a cluster is enriched for amino acid residues comprising a hydrophobic side chain at X¹⁰. In some embodiments, X¹⁰ is M. In some embodiments, the side chain of X¹⁰ is aliphatic. In some embodiments, the side chain of X¹⁰ is C₁₋₆ alkyl. In some embodiments, X¹⁰ is A. In some embodiments, X¹⁰ is L. In some embodiments, X¹⁰ is I. In some embodiments, X¹⁰ comprises a side chain comprising an aromatic group. In some embodiments, X¹⁰ is F. In some embodiments, X¹⁰ is Y. In some embodiments, X¹⁰ is W.

Various amino acid residues may be utilized for X¹³. For example, in some embodiments, the side chain of X¹³ is hydrophobic. In some embodiments, a cluster is enriched for amino acid residues comprising a hydrophobic side chain at X¹³. In some embodiments, the side chain of X¹³ is aliphatic. In some embodiments, the side chain of X¹³ is C₁₋₆ alkyl. In some embodiments, X¹³ is A. In some embodiments, X¹³ is V. In some embodiments, X¹³ is I. In some embodiments, X¹³ is L.

In some embodiments, an agent comprising a cluster C78 sequence is capable of binding to CUL1.

### Cluster C79

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C79 as described herein. In some embodiments, an agent comprises X⁷⁹X²X³X⁴X⁵X⁶X⁷X⁸X⁹X¹⁰X¹¹X¹²X¹³X¹⁴ wherein each of X⁷⁹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁷⁹X²X³X⁴X⁵X⁶X⁷X⁸X⁹X¹⁰X¹¹X¹²X¹³X¹⁴,

wherein:
each of X⁷⁹, X², X³, X⁴, X⁵, X⁶, X⁷, X⁸, X⁹, X¹⁰, X¹¹, X¹², X¹³, and X¹⁴ is independently an amino acid residue;
X¹³ comprises a hydrophobic side chain or a side chain comprising an aromatic group; and
X¹⁴ comprises a side chain comprising an aromatic group.

Various amino acid residues may be utilized for X². For example, in some embodiments, the side chain of X² is hydrophobic. In some embodiments, a cluster is enriched for amino acid residues comprising a hydrophobic side chain at X². In some embodiments, X² is M. In some embodiments, the side chain of X² is aliphatic. In some embodiments, the side chain of X² is C₁₋₆ alkyl. In some embodiments, X² is I. In some embodiments, X² is L. In some embodiments, X² comprises a side chain comprising an aromatic group. In some embodiments, X² is F. In some embodiments, X² is Y. In some embodiments, X² is W.

Various amino acid residues may be utilized for X³. For example, in some embodiments, the side chain of X³ is hydrophobic. In some embodiments, a cluster is enriched for amino acid residues comprising a hydrophobic side chain at X³. In some embodiments, X³ is M. In some embodiments, the side chain of X³ is aliphatic. In some embodiments, the side chain of X³ is C₁₋₆ alkyl. In some embodiments, X³ is A. In some embodiments, X³ is V. In some embodiments, X³ is I. In some embodiments, X³ comprises a side chain comprising an aromatic group. In some embodiments, X³ is F. In some embodiments, X³ is Y. In some embodiments, X³ is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

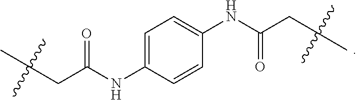 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprise an acidic group at X⁵. In some embodiments, X⁵ comprises a side chain comprising —COOH. In some embodiments, X⁵ is D. In some embodiments, X⁵ is E.

Various amino acid residues may be utilized for X⁶. For example, in some embodiments, X⁶ comprises a side chain comprising an acidic group. In some embodiments, a cluster is enriched for amino acid residues whose side chains comprise an acidic group at X⁶. In some embodiments, X⁶ comprises a side chain comprising —COOH. In some embodiments, X⁶ is D. In some embodiments, X⁶ is E. In some embodiments, X⁶ comprises a side chain comprising a polar group. In some embodiments, a cluster is enriched for amino acid residues whose side chains comprise a polar group at X⁶. In some embodiments, X⁶ comprises a side chain comprising —C(O)NH₂. In some embodiments, X⁶ is N.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising an acidic group. In some embodiments, a cluster is enriched for amino acid residues whose side chains comprise an acidic group at X⁹. In some embodiments, X⁹ comprises a side chain comprising —COOH. In some embodiments, X⁹ is D. In some embodiments, X⁹ is E. In some embodiments, the side chain of X⁹ is hydrophobic. In some embodiments, a cluster is enriched for amino acid residues comprising a hydrophobic side chain at X⁹. In some embodiments, the side chain of X⁹ is aliphatic. In some embodiments, the side chain of X⁹ is C₁₋₆ alkyl. In some embodiments, X⁹ is L.

Various amino acid residues may be utilized for X¹⁰. For example, in some embodiments, the side chain of X¹⁰ is hydrophobic. In some embodiments, a cluster is enriched for amino acid residues comprising a hydrophobic side chain at X¹⁰. In some embodiments, the side chain of X¹⁰ is aliphatic. In some embodiments, the side chain of X¹⁰ is C₁₋₆ alkyl. In some embodiments, X¹⁰ is I. In some embodiments, X¹⁰ is L.

Various amino acid residues may be utilized for X¹². For example, in some embodiments, X¹² comprises a side chain comprising an acidic group. In some embodiments, a cluster is enriched for amino acid residues whose side chains comprise an acidic group at X¹². In some embodiments, X¹² comprises a side chain comprising —COOH. In some embodiments, X¹² is D. In some embodiments, X¹² is E.

Various amino acid residues may be utilized for X¹³. For example, in some embodiments, the side chain of X¹³ is aliphatic. In some embodiments, the side chain of X¹³ is C₁₋₆ alkyl. In some embodiments, X¹³ is A. In some embodiments, X¹³ comprises a side chain comprising an aromatic group. In some embodiments, X¹³ is F. In some embodiments, X¹³ is W.

Various amino acid residues may be utilized for X¹⁴. In some embodiments, X¹⁴ comprises a side chain comprising an aromatic group. In some embodiments, X¹⁴ is F. In some embodiments, X¹⁴ is W.

In some embodiments, an agent comprising a cluster C79 sequence is capable of binding to FBXW7.

### RING/U-BOX Family

### Cluster C80

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C80 as described herein. In some embodiments, an agent comprises X⁸⁰X²X³X⁴X⁵X⁶X⁷X⁸X⁹X¹⁰X¹¹X¹²X¹³X¹⁴, wherein each of X⁸⁰, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

[X⁸⁰]p1[X²]p2[X³]p3X⁴X⁵X⁶X⁷X⁸X⁹X¹⁰X¹¹X¹²X³X⁴

wherein:
each of p1, p2 and p3 is independently 0 or 1;
each of X⁸⁰, X², X³, X⁴, X⁵, X⁶, X⁷, X⁸, X⁹, X¹⁰, X¹¹, X¹², X¹³, and X¹⁴ is independently an amino acid residue;
X⁷ comprises a hydrophobic side chain; and
X⁹ comprises a hydrophobic side chain.

Various amino acid residues may be utilized for X⁸⁰. In some embodiments, X⁸⁰ is absent. In some embodiments, X⁸⁰ is P.

Various amino acid residues may be utilized for X². In some embodiments, X² is absent. In some embodiments, X² is P.

Various amino acid residues may be utilized for X³. In some embodiments, X³ is absent. In some embodiments, X² is P. In some embodiments, X² comprises a hydrophobic side chain. In some embodiments, X² is I.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

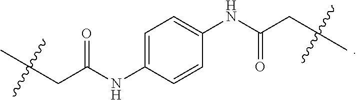 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ is comprises a side chain comprising an aromatic group. In some embodiments, X⁵ is W. In some embodiments, X⁵ is Y. In some embodiments, X⁵ is F.

Various amino acid residues may be utilized for X⁶. For example, in some embodiments, X⁶ comprises a side chain comprising an acidic group. In some embodiments, a cluster is enriched for amino acid residues whose side chains comprise acidic groups at X⁶. In some embodiments, X⁶ comprises a side chain comprising —COOH. In some embodiments, X⁶ is D. In some embodiments, X⁶ is E. In some embodiments, X⁶ comprises a side chain comprising a polar group. In some embodiments, a cluster is enriched for amino acid residues whose side chains comprise a polar group at X⁶. In some embodiments, X⁶ comprises a side chain comprising —OH. In some embodiments, X⁶ is S. In some embodiments, X⁶ is T. In some embodiments, X⁶ comprises a side chain comprising —C(O)NH₂. In some embodiments, X⁶ is N. In some embodiments, X⁶ is Q.

Various amino acid residues may be utilized for X⁷. For example, in some embodiments, the side chain of X⁷ is aliphatic. In some embodiments, the side chain of X⁷ is C₁₋₆ alkyl. In some embodiments, X⁷ is A.

Various amino acid residues may be utilized for X⁸. For example, in some embodiments, the side chain of X⁸ is hydrophobic. In some embodiments, a cluster is enriched for amino acid residues comprising a hydrophobic side chain at X⁸. In some embodiments, X⁸ comprises a side chain comprising an aromatic group. In some embodiments, X⁸ is W.

Various amino acid residues may be utilized for X⁹. For example, in some embodiments, X⁹ is M. In some embodiments, the side chain of X⁹ is aliphatic. In some embodiments, the side chain of X⁹ is C₁₋₆ alkyl. In some embodiments, X⁹ is V. In some embodiments, X⁹ is I. In some embodiments, X⁹ is L.

Various amino acid residues may be utilized for X¹⁰. For example, in some embodiments, X¹⁰ comprises a side chain comprising an acidic group. In some embodiments, a cluster is enriched for amino acid residues whose side chains comprise an acidic group at X¹⁰. In some embodiments, X¹⁰ comprises a side chain comprising —COOH. In some embodiments, X¹⁰ is E.

Various amino acid residues may be utilized for X¹². For example, in some embodiments, X¹² comprises a side chain comprising an acidic group. In some embodiments, a cluster is enriched for amino acid residues whose side chains comprise an acidic group at X¹². In some embodiments, X¹² comprises a side chain comprising —COOH. In some embodiments, X¹² is D. In some embodiments, X¹² comprises a side chain comprising a polar group. In some embodiments, a cluster is enriched for amino acid residues whose side chains comprise a polar group at X¹². In some embodiments, X¹² comprises a side chain comprising —OH. In some embodiments, X¹² is S. In some embodiments, X¹² is T.

In some embodiments, an agent described herein, e.g., a stapled peptide, interacts with one or more (e.g., 1, 2, 3, 4, 5, 6, 7, 8, 9, 10, 11, 12, 13, 14, 15, 16, 17, 18, 19, 20, 21, or 22) or all of the following amino acid residues of CHIP or amino acid residues corresponding thereto: Phe37, Val38, Tyr49, Val61, Asn65, Leu68, Leu71, Lys72, Ser93, Val94, Lys95, Phe98, Phe99, Gln102, Glu106, Gln127, Leu129, Asn130, Phe131, Gly132, Asp134, and Ile135. In some embodiments, it interacts with Asn34 of CHIP or an amino acid residue corresponding thereto. In some embodiments, it interacts with Phe37 of CHIP or an amino acid residue corresponding thereto. In some embodiments, it interacts with Val38 of CHIP or an amino acid residue corresponding thereto. In some embodiments, it interacts with Tyr49 of CHIP or an amino acid residue corresponding thereto. In some embodiments, it interacts with Asn65 of CHIP or an amino acid residue corresponding thereto. In some embodiments, it interacts with Leu68 of CHIP or an amino acid residue corresponding thereto. In some embodiments, it interacts with Leu71 of CHIP or an amino acid residue corresponding thereto. In some embodiments, it interacts with Lys72 of CHIP or an amino acid residue corresponding thereto. In some embodiments, it interacts with Ser93 of CHIP or an amino acid residue corresponding thereto. In some embodiments, it interacts with Val94 of CHIP or an amino acid residue corresponding thereto. In some embodiments, it interacts with Lys95 of CHIP or an amino acid residue corresponding thereto. In some embodiments, it interacts with Phe98 of CHIP or an amino acid residue corresponding thereto. In some embodiments, it interacts with Phe99 of CHIP or an amino acid residue corresponding thereto. In some embodiments, it interacts with Gln102 of CHIP or an amino acid residue corresponding thereto. In some embodiments, it interacts with Glu106 of CHIP or an amino acid residue corresponding thereto. In some embodiments, it interacts with Leu129 of CHIP or an amino acid residue corresponding thereto. In some embodiments, it interacts with Asn130 of CHIP or an amino acid residue corresponding thereto. In some embodiments, it interacts with Phe131 of CHIP or an amino acid residue corresponding thereto. In some embodiments, it interacts with Gly132 of CHIP or an amino acid residue corresponding thereto. In some embodiments, it interacts with Asp134 of CHIP or an amino acid residue corresponding thereto. In some embodiments, it interacts with Ile135 of CHIP or an amino acid residue corresponding thereto.

In some embodiments, an agent comprising a cluster C80 sequence is capable of binding to CHIP.

### Cluster C81

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C81 as described herein. In some embodiments, an agent comprises X⁸¹X²X³X⁴X⁵X⁶X⁷X⁸X⁹X¹⁰X¹¹X¹²X³X¹⁴, wherein each of X⁸¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁸¹X²X³X⁴X⁵X⁶X⁷X⁸X⁹X¹⁰X¹¹X¹²X¹³X¹⁴,

wherein:
each of X⁸¹, X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain or a side chain comprising an aromatic group;
X⁶ comprises a side chain comprising a polar group;
X¹⁰ comprises a side chain comprising an acidic group; and
X¹⁴ comprises a hydrophobic side chain or a side chain comprising an aromatic group.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

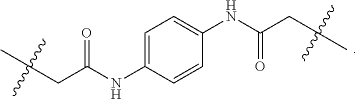 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ comprises a side chain comprising an aromatic group. In some embodiments, X⁵ is F.

Various amino acid residues may be utilized for X⁶. For example, in some embodiments, X⁶ comprises a side chain comprising a polar group. In some embodiments, a cluster is enriched for amino acid residues whose side chains comprise a polar group at X⁶. In some embodiments, X⁶ comprises a side chain comprising —OH. In some embodiments, X⁶ is S. In some embodiments, X⁶ is T. In some embodiments, X⁶ comprises a side chain comprising —C(O)NH₂. In some embodiments, X⁶ is Q.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ is hydrophobic. In some embodiments, a cluster is enriched for amino acid residues comprising a hydrophobic side chain at X⁹. In some embodiments, X⁹ comprises a side chain comprising an aromatic group. In some embodiments, X⁹ is W.

Various amino acid residues may be utilized for X¹⁰. For example, in some embodiments, X¹⁰ comprises a side chain comprising —COOH. In some embodiments, X¹⁰ is D.

Various amino acid residues may be utilized for X¹². For example, in some embodiments, X¹² comprises a side chain comprising a polar group. In some embodiments, a cluster is enriched for amino acid residues whose side chains comprise a polar group at X¹². In some embodiments, X¹² comprises a side chain comprising —C(O)NH₂. In some embodiments, X¹² is Q.

Various amino acid residues may be utilized for X¹³. For example, in some embodiments, the side chain of X¹³ is hydrophobic. In some embodiments, a cluster is enriched for amino acid residues comprising a hydrophobic side chain at X¹³. In some embodiments, X¹³ comprises a side chain comprising an aromatic group. In some embodiments, X¹³ is F. In some embodiments, X¹³ is Y.

Various amino acid residues may be utilized for X¹⁴. For example, in some embodiments, X¹⁴ comprises a side chain comprising an aromatic group. In some embodiments, X¹⁴ is M. In some embodiments, X¹⁴ comprises a side chain comprising an aromatic group. In some embodiments, X¹⁴ is F. In some embodiments, X¹⁴ is W.

In some embodiments, an agent comprising a cluster C81 sequence is capable of binding to MDM2.

### Cluster C82

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C82 as described herein. In some embodiments, an agent comprises X⁸²X²X³X⁴X⁵X⁶X⁷X⁸X⁹X¹⁰X¹¹X¹²X³X¹⁴, wherein each of X⁸², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁸²X²X³X⁴X⁵X⁶X⁷X⁸X⁹X¹⁰X¹¹X¹²X¹³X¹⁴,

wherein:
each of X⁸², X², X³, X⁴, X⁵, X⁶, X⁷, X⁸, X⁹, X¹⁰, X¹¹, X¹², X¹³, and X¹⁴ is independently an amino acid residue;
X⁹ comprises a hydrophobic side chain or a side chain comprising an aromatic group; and
X¹³ comprises a hydrophobic side chain or a side chain comprising an aromatic group.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

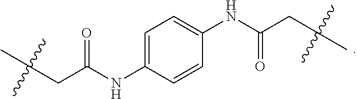 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a polar group. In some embodiments, a cluster is enriched for amino acid residues whose side chains comprise a polar group at X⁵. In some embodiments, X⁵ comprises a side chain comprising —OH. In some embodiments, X⁵ is S. In some embodiments, X⁵ is T. In some embodiments, X⁵ comprises a side chain comprising —C(O)NH₂. In some embodiments, X⁵ is N. In some embodiments, X⁵ is Q.

Various amino acid residues may be utilized for X⁶. For example, in some embodiments, X⁶ comprises a side chain comprising an acidic group. In some embodiments, a cluster is enriched for amino acid residues whose side chains comprise an acidic group at X⁶. In some embodiments, X⁶ comprises a side chain comprising —COOH. In some embodiments, X⁶ is D. In some embodiments, X⁶ is E.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ is hydrophobic. In some embodiments, a cluster is enriched for amino acid residues comprising a hydrophobic side chain at X⁹. In some embodiments, X⁹ comprises a side chain comprising an aromatic group. In some embodiments, X⁹ is W. In some embodiments, X⁹ is F.

Various amino acid residues may be utilized for X¹⁰. For example, in some embodiments, the side chain of X¹⁰ is hydrophobic. In some embodiments, a cluster is enriched for amino acid residues comprising a hydrophobic side chain at X¹⁰. In some embodiments, X¹⁰ comprises a side chain comprising an aromatic group. In some embodiments, X¹⁰ is W.

Various amino acid residues may be utilized for X¹². For example, in some embodiments, the side chain of X¹² is hydrophobic. In some embodiments, a cluster is enriched for amino acid residues comprising a hydrophobic side chain at X¹². In some embodiments, X¹² comprises a side chain comprising an aromatic group. In some embodiments, X¹² is H. In some embodiments, X¹² is F. In some embodiments, X¹² is Y. In some embodiments, X¹² is W.

Various amino acid residues may be utilized for X¹³. In some embodiments, X¹³ comprises a side chain comprising an aromatic group. In some embodiments X¹³ is W.

Various amino acid residues may be utilized for X¹⁴. For example, in some embodiments, the side chain of X¹⁴ is hydrophobic. In some embodiments, a cluster is enriched for amino acid residues comprising a hydrophobic side chain at X¹⁴. In some embodiments, X¹⁴ is M. In some embodiments, the side chain of X¹⁴ is aliphatic. In some embodiments, the side chain of X¹⁴ is C₁₋₆ alkyl. In some embodiments, X¹⁴ is V. In some embodiments, X¹⁴ is I. In some embodiments, X¹⁴ comprises a side chain comprising an aromatic group. In some embodiments, X¹⁴ is F. In some embodiments, X¹⁴ is Y. In some embodiments, X¹⁴ is W.

In some embodiments, an agent comprising a cluster C82 sequence is capable of binding to MDM2.

### Cluster C83

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C83 as described herein. In some embodiments, an agent comprises X⁸³X²X³X⁴X⁵X⁶X⁷X⁸X⁹X¹⁰X¹¹X¹²X¹³X¹⁴, wherein each of X⁸³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁸³X²X³X⁴X⁵X⁶X⁷X⁸X⁹X¹¹X¹²X¹³X¹⁴,

wherein:
each of X⁸³, X², X³, X⁴, X⁵, X⁶, X⁷, X⁸, X⁹, X¹⁰, X¹¹, X¹², X¹³, and X¹⁴ is independently an amino acid residue;
X⁸³ comprises a hydrophobic side chain or a side chain comprising an aromatic group;
X⁵ comprises a side chain comprising a polar group; and
X⁹ comprises a hydrophobic side chain.

Various amino acid residues may be utilized for X⁸³. For example, in some embodiments, X⁸³ comprises a side chain comprising an aromatic group. In some embodiments, a cluster is enriched for amino acid residues whose side chains comprise an aromatic group at X⁸³. In some embodiments, X⁸³ is W.

Various amino acid residues may be utilized for X². For example, in some embodiments, X² comprises a side chain comprising an acidic group. In some embodiments, a cluster is enriched for amino acid residues whose side chains comprise an acidic group at X². In some embodiments, X² comprises a side chain comprising —COOH. In some embodiments, X² is D. In some embodiments, X² is E. In some embodiments, X² comprises a side chain comprising an aromatic group. In some embodiments, X² is W.

Various amino acid residues may be utilized for X³. For example, in some embodiments, X³ comprises a side chain comprising a basic group. In some embodiments, a cluster is enriched for amino acid residues whose side chains comprise a basic group at X³. In some embodiments, X³ is R.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

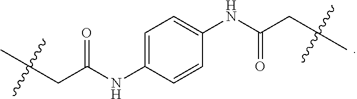 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ is Q.

Various amino acid residues may be utilized for X⁶. For example, in some embodiments, the side chain of X⁶ is hydrophobic. In some embodiments, a cluster is enriched for amino acid residues comprising a hydrophobic side chain at X⁶. In some embodiments, X⁶ comprises a side chain comprising an aromatic group. In some embodiments, X⁶ is F. In some embodiments, X⁶ is Y. In some embodiments, X⁶ is W.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, the side chain of X⁹ is aliphatic. In some embodiments, the side chain of X⁹ is C₁₋₆ alkyl. In some embodiments, X⁹ is A. In some embodiments, X⁹ is V. In some embodiments, X⁹ is I.

Various amino acid residues may be utilized for X¹⁰. For example, in some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —OH. In some embodiments, X¹⁰ is S. In some embodiments, X¹⁰ is T. In some embodiments, the side chain of X¹⁰ is hydrophobic. In some embodiments, a cluster is enriched for amino acid residues comprising a hydrophobic side chain at X¹⁰. In some embodiments, X¹⁰ is M.

In some embodiments, an agent comprising a cluster C83 sequence is capable of binding to CHIP.

### Cluster C84

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C84 as described herein. In some embodiments, an agent comprises X⁸⁴X²X³X⁴X⁵X⁶X⁷X⁸X⁹X¹⁰X¹¹X¹²X¹³X¹⁴, wherein each of X⁸⁴, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁸⁴X²X³X⁴X⁵X⁶X⁷X⁸X⁹X¹⁰X¹¹X¹²X¹³X¹⁴,

wherein:
each of X⁸⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain;
X¹³ comprises a hydrophobic side chain; and
X¹⁴ comprises a side chain comprising an acidic group.

Various amino acid residues may be utilized for X³. For example, in some embodiments, the side chain of X³ is hydrophobic. In some embodiments, a cluster is enriched for amino acid residues comprising a hydrophobic side chain at X³. In some embodiments, X³ is M. In some embodiments, the side chain of X³ is aliphatic. In some embodiments, the side chain of X³ is C₁₋₆ alkyl. In some embodiments, X³ is A. In some embodiments, X³ is V. In some embodiments, X³ is I. In some embodiments, X³ comprises a side chain comprising an aromatic group. In some embodiments, X³ is F. In some embodiments, X³ is Y. In some embodiments, X³ is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

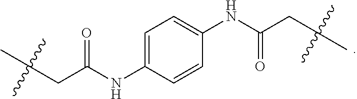 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a polar group. In some embodiments, a cluster is enriched for amino acid residues whose side chains comprise a polar group at X⁵. In some embodiments, X⁵ comprises a side chain comprising —OH. In some embodiments, X⁵ is S. In some embodiments, X⁵ is T. In some embodiments, X⁵ comprises a side chain comprising a basic group. In some embodiments, a cluster is enriched for amino acid residues whose side chains comprise a basic group at X⁵. In some embodiments, X⁵ comprises a side chain comprising an aromatic group. In some embodiments, X⁵ is H. In some embodiments, X⁵ is F. In some embodiments, X⁵ is Y. In some embodiments, X⁵ is W.

Various amino acid residues may be utilized for X⁶. For example, in some embodiments, the side chain of X⁶ is hydrophobic. In some embodiments, a cluster is enriched for amino acid residues comprising a hydrophobic side chain at X⁶. In some embodiments, X⁶ comprises a side chain comprising an aromatic group. In some embodiments, X⁶ is F. In some embodiments, X⁶ is Y. In some embodiments, X⁶ is W.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising a basic group. In some embodiments, a cluster is enriched for amino acid residues whose side chains comprise a basic group at X⁹. In some embodiments, X⁹ is H. In some embodiments, X⁹ comprises a side chain comprising an acidic group. In some embodiments, a cluster is enriched for amino acid residues whose side chains comprise an acidic group at X⁹. In some embodiments, X⁹ comprises a side chain comprising —COOH. In some embodiments, X⁹ is D. In some embodiments, X⁹ comprises a side chain comprising a polar group. In some embodiments, a cluster is enriched for amino acid residues whose side chains comprise a polar group at X⁹. In some embodiments, X⁹ comprises a side chain comprising —OH. In some embodiments, X⁹ is S. In some embodiments, X⁹ comprises a side chain comprising —C(O)NH₂. In some embodiments, X⁹ is N. In some embodiments, X⁹ is Q.

Various amino acid residues may be utilized for X¹⁰. In some embodiments, X¹⁰ is I.

Various amino acid residues may be utilized for X¹². For example, in some embodiments, the side chain of X¹² is hydrophobic. In some embodiments, a cluster is enriched for amino acid residues comprising a hydrophobic side chain at X¹². In some embodiments, X¹² is M. In some embodiments, X¹² comprises a side chain comprising an acidic group. In some embodiments, a cluster is enriched for amino acid residues whose side chains comprise an acidic group at X¹². In some embodiments, X¹² comprises a side chain comprising —COOH. In some embodiments, X¹² is D. In some embodiments, X¹² comprises a side chain comprising an aromatic group. In some embodiments, X¹² is W.

Various amino acid residues may be utilized for X¹³. For example, in some embodiments, the side chain of X¹³ is aliphatic. In some embodiments, the side chain of X¹³ is C₁₋₆ alkyl. In some embodiments, X¹³ is V. In some embodiments, X¹³ is I.

Various amino acid residues may be utilized for X¹⁴. In some embodiments, X¹⁴ is D.

In some embodiments, an agent comprising a cluster C84 sequence is capable of binding to CHIP.

### Cluster C85

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C85 as described herein. In some embodiments, an agent comprises X⁸⁵X²X³X⁴X⁵X⁶X⁷X⁸X⁹X¹⁰X¹¹X¹²X¹³X¹⁴, wherein each of X⁸⁵, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁸⁵X²X³X⁴X⁵X⁶X⁷X⁸X⁹X¹⁰X¹¹X¹²X¹³X¹⁴,

wherein:
each of X⁸⁵, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising an acidic or polar group;
X⁵ comprises a hydrophobic side chain or a side chain comprising an aromatic group;
X⁹ comprises a hydrophobic side chain or a side chain comprising an aromatic group; and
X¹² comprises a hydrophobic side chain.

Various amino acid residues may be utilized for X³. For example, in some embodiments, a cluster is enriched for amino acid residues whose side chains comprise an acidic group at X³. In some embodiments, X³ comprises a side chain comprising —COOH or a salt form thereof. In some embodiments, X³ is D. In some embodiments, X³ is E. In some embodiments, X³ comprises a side chain comprising a polar group. In some embodiments, a cluster is enriched for amino acid residues whose side chains comprise a polar group at X³. In some embodiments, X³ comprises a side chain comprising -C(O)NH₂. In some embodiments, X³ is N. In some embodiments, X³ is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

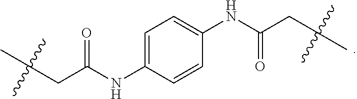 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ is comprises a side chain comprising an aromatic group. In some embodiments, X⁵ is F.

Various amino acid residues may be utilized for X⁶. For example, in some embodiments, X⁶ comprises a side chain comprising a polar group. In some embodiments, a cluster is enriched for amino acid residues whose side chains comprise a polar group at X⁶. In some embodiments, X⁶ comprises a side chain comprising —OH. In some embodiments, X⁶ is S. In some embodiments, X⁶ is T. In some embodiments, X⁶ comprises a side chain comprising —C(O)NH₂. In some embodiments, X⁶ is N. In some embodiments, X⁶ is Q.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ is comprises a side chain comprising an aromatic group. In some embodiments, X⁹ is W.

Various amino acid residues may be utilized for X¹². For example, in some embodiments, X¹² is M. In some embodiments, the side chain of X¹² is aliphatic. In some embodiments, the side chain of X¹² is C₁₋₆ alkyl. In some embodiments, X¹² is A. In some embodiments, X¹² is V. In some embodiments, X¹² is I.

Various amino acid residues may be utilized for X¹³. For example, in some embodiments, the side chain of X¹³ is hydrophobic. In some embodiments, a cluster is enriched for amino acid residues comprising a hydrophobic side chain at X¹³. In some embodiments, X¹³ is M. In some embodiments, the side chain of X¹³ is aliphatic. In some embodiments, the side chain of X¹³ is C₁₋₆ alkyl. In some embodiments, X¹³ is A. In some embodiments, X¹³ is V. In some embodiments, X¹³ is I. In some embodiments, X¹³ comprises a side chain comprising an aromatic group. In some embodiments, X¹³ is W.

In some embodiments, an agent comprising a cluster C85 sequence is capable of binding to MDM2.

### Trimerizers

In some embodiments, the present disclosure provides technologies for identification, characterization, production, and/or use of stapled peptide agents that can generate or enhance interactions of multiple targets of interest. In some embodiments, stapled peptide agents can bridge two targets of interests. In some embodiments, stapled peptide agents can enhance interactions of two targets of interest. In some embodiments, two targets of interest do not interact with each other absence of stapled peptide agents. In some embodiments, absence of provided stapled peptide agents, interactions of the two targets of interest are of low level or cannot be detected. In some embodiments, two targets of interest and a stapled peptide form a trimer (trimerize). In some embodiments, a stapled peptide forms a trimer with two targets of interest (such a stapled peptide may be referred to as a “trimerizer” or “molecular glue”).

In some embodiments, a trimerizer interacts with two targets of interest to cooperatively bind the targets of interest (e.g., polypeptides) together. In some embodiments, a trimerizer interacts with two protein surfaces to cooperatively bind the proteins together. In some embodiments, a trimerizer can bind targets of interest with greater affinity. In some embodiments, a trimerizer can bind sites or targets that a typical agent cannot bind. In some embodiments, a trimerizer is useful for inducing protein degradation.

In some embodiments, the present disclosure provides technologies for identifying trimerizers.

In some embodiments, the present disclosure provides a method comprising:
contacting a first collection of stapled peptides with a first target of interest so that a number of stapled peptides of the first collection binds to the target;
collecting stapled peptides of the first collection that bind to the first target;
contacting the collected stapled peptides with a second target of interest so that one or more stapled peptides binds to the second target; and
determining amino acid sequences of stapled peptides that bind to the second target.

In some embodiments, contact with a second target of interest is performed in the presence of a first target of interest. In some embodiments, contact in the presence of both targets of interest reduce or prevent enrichment of stapled peptides that can bind to a second target of interest but cannot bridge both target of interest.

In some embodiments, after contacting a first collection of stapled peptides with a first target of interest, sequences of stapled peptides of the first collection that bind to the first target are determined, and a second collection of stapled peptides is generated based on sequences of stapled peptides of the first collection that bind to the first target. For example, in some embodiments, enriched amino acid residues shared by a cluster of stapled peptides of the first collection that bind to the first target at one or more or all positions are determined, and when generating a second collection of stapled peptides, such enriched amino acid residues c utilized at one or more or all corresponding positions. At each position where enrichment is observed, one or more of the enriched amino acid residues at such a position based on the first collection can be independently and optionally utilized. In some embodiments, at each position that is important for interacting with a first target of interest, one or more of the enriched amino acid residues at such a position based on the first collection are independently and optionally utilized. In some embodiments, at each position that is important for interacting with a first target of interest, one or more of the enriched amino acid residues at such a position based on the first collection are independently utilized.

In some embodiments, the present disclosure provides comprising:
contacting a first collection of stapled peptides with a first target of interest so that a number of stapled peptides of the first collection binds to the target;
determining amino acid sequences of stapled peptides of the first collection that bind to the first target;
identifying one or more enriched amino acid residues shared by a cluster of stapled peptides of the first collection that bind to the first target independently at one or more enriched positions (“first collection enriched amino acid residues”);
contacting a second collection of stapled peptides with a second target of interest so that one or more stapled peptides of the second collection binds to the second target, wherein the second collection is enriched for one or more first collection enriched amino acid residues independently at one or more enriched positions or positions corresponding thereto; and
determining amino acid sequences of stapled peptides that bind to the second target.

In some embodiments, the present disclosure provides a method, comprising:
contacting a first collection of stapled peptides with a first target of interest so that a number of stapled peptides of the first collection binds to the target;
determining amino acid sequences of stapled peptides of the first collection that bind to the first target;
identifying one or more enriched amino acid residues shared by a cluster of stapled peptides of the first collection that bind to the first target independently at one or more enriched positions (“first collection enriched amino acid residues”); providing a second collection of stapled peptides, which stapled peptides are enriched for one or more of the enriched amino acid residue independently at one or more enriched positions or positions corresponding thereto, and are randomized at one or more positions;
contacting a second collection of stapled peptides with a second target of interest so that one or more stapled peptides of the second collection binds to the second target; and
determining amino acid sequences of stapled peptides that bind to the second target.

In some embodiments, the present disclosure provides a method, comprising:
contacting a collection of stapled peptides with a target of interest so that a number of stapled peptides of the collection binds to the target, wherein the collection is enriched for one or more enriched amino acid residues independently at one or more enriched positions; and
determining amino acid sequences of stapled peptides that bind to the target; wherein the one or more enriched amino acid residues are characterized in that when a collection of stapled peptides, which are randomized independently at the one or more enriched positions or positions corresponding thereto, is contacted with another target of interest, the stapled peptides that bind to the another target is enriched for one or more of the enriched amino acid residues independently at one or more of the enriched positions.

In some embodiments, a target of interest is a second target of interest as described herein. In some embodiments, an another target of interest is a first target of interest as described herein.

In some embodiments, first collection enriched amino acid residues are identified from a first collection of stapled peptides. In some embodiments, one or more or all of such enriched amino acid residues are utilized to generate a second collection of stapled peptides, biased/enriched for one or more or all of the enriched amino acid residues at relevant positions. In some embodiments, each of the first collection enriched amino acid residues is preferred or required for binding to a first target of interest. In some embodiments, each of the first collection enriched amino acid residues biased/enriched for in a second collection is preferred or required for binding to a first target of interest. In some embodiments, contacting with a second target of interest is performed in the presence of the first target. In some embodiments, stapled peptides that can bridge the first and second targets of interest is selected over stapled peptides that only binds to a second target of interest, only binds to a second target of interest, or can bind to both the first and the second targets of interest but cannot bridge them together. In some embodiments, a stapled peptide is a trimerizer. In some embodiments, a first target of interest is cytosolic. In some embodiments, a first target of interest is more abundant than a second target of interest. In some embodiments, a first target of interest is a polypeptide. In some embodiments, a first target of protein is considered an abundant protein by those skilled in the art. In some embodiments, a first target of interest may be referred to as a presenter protein. In some embodiments, a second target of interest is a polypeptide. In some embodiments, one of the target of interest is an E3 ubiquitin ligase, e.g., MDM2. In some embodiments, a presenter protein is an E3 ubiquitin ligase. In some embodiments, the other target of interest is degraded.

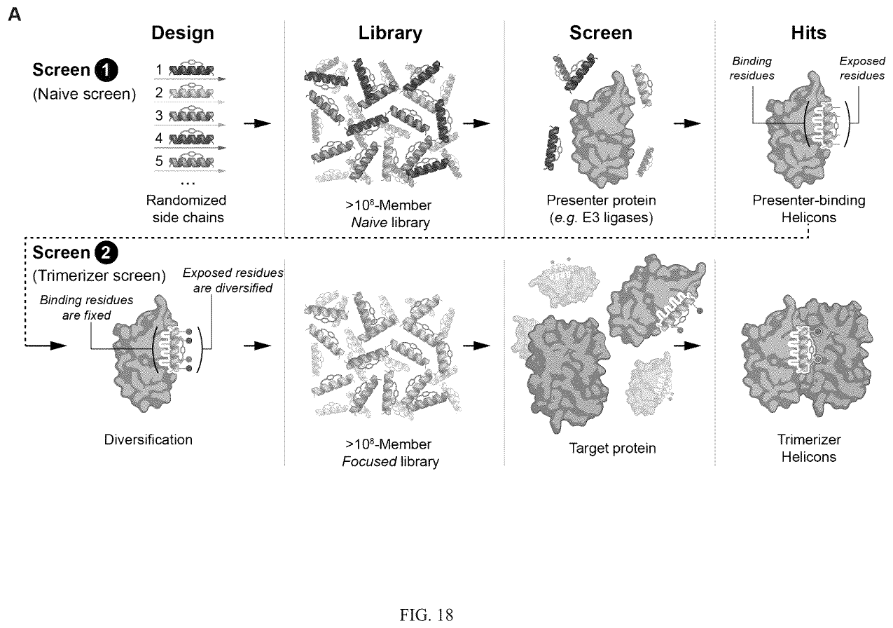

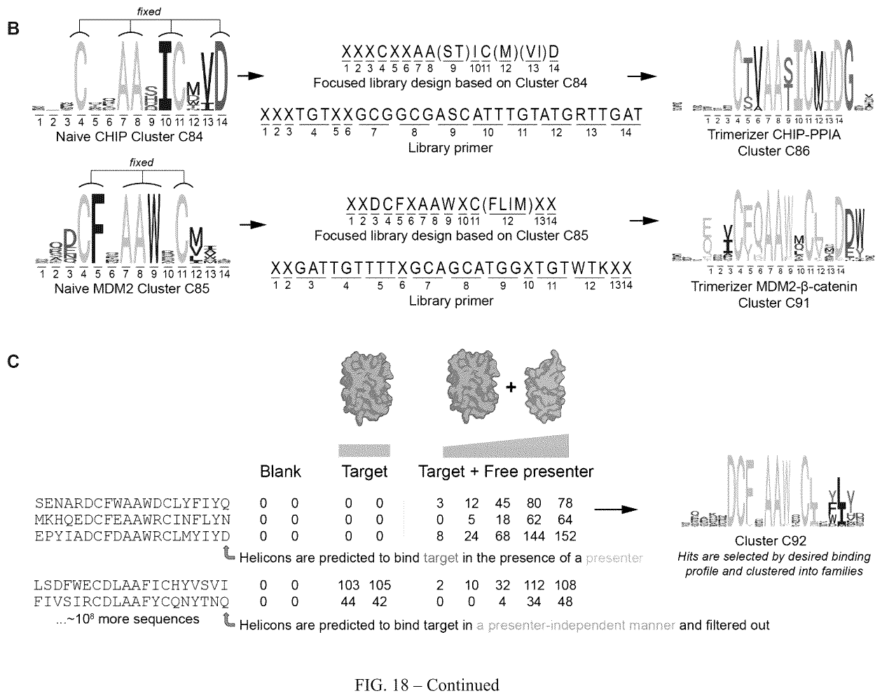

For example, in some embodiments, a first collection of stapled peptides is or comprises a naive library targeting a first target of interest. In some embodiments, a first target of interest is or comprises a polypeptide. In some embodiments, a first target of interest is or comprises an E3 ligase. In some embodiments, a first target of interest is CHIP. In some embodiments, a first target of interest is MDM2. In some embodiments, a first collection enriched amino acid residues comprises enriched amino acid residues from a cluster, e.g., enriched amino acid residues in various clusters as described herein (see, e.g., cluster C84 and C85 in FIG. 18, (B)). In some embodiments, a second target of interest is or comprises a polypeptide. In some embodiments, a second target of interest is or comprises a target polypeptide as described herein. In some embodiments, a second target of interest is or comprises a polypeptide with which a first target of interest form a complex. In some embodiments, a second target of interest is or comprises a polypeptide with which a first target of interest interact. For example, in some embodiments, a second target of interest is PPIA; in some embodiments, a second target of interest is beta-catenin. Certain example technologies are described in the Examples.

In some embodiments, the present disclosure provides a method for reducing level of a polypeptide, e.g., beta-catenin, comprising administering to a system comprising the peptide a stapled peptide, wherein the stapled peptide bridges the polypeptide to an E3 ubiquitin ligase. In some embodiments, a present disclosure provides trimerizers for beta-catenin and MDM2.

Among other things, provided technologies can recognize a polypeptide surface. In some embodiments, provided technologies can reprogram a polypeptide surface. In some embodiments, the present disclosure provides a method for recognizing or reprogramming a polypeptide surface, comprising contacting the polypeptide with an agent of the present disclosure. In some embodiments, contacting is performed in the presence of another polypeptide, wherein the agent binds to the polypeptide and the another polypeptide cooperatively. In some embodiments, the present disclosure provides a method for reprograming a polypeptide to cooperatively bind a target polypeptide, comprising contacting the polypeptides with an agent present disclosure.

In some embodiments, provided technologies can modulate polypeptide interactions. In some embodiments, the provided technologies can enhance certain interactions. In some embodiments, the provided technologies can induce new interactions. In some embodiments, provided technologies inhibit certain interactions. In some embodiments, provided technologies remove certain interactions. In some embodiments, the present disclosure provides a method for modulating polypeptide interaction, comprising contacting two polypeptides with an agent of present disclosure, wherein the agent binds to the two polypeptides cooperatively.

In some embodiments, the present disclosure provides technologies for modulating polypeptide functions. In some embodiments, the present disclosure provides technologies for modulating polypeptide levels. In some embodiments, the present disclosure provides technologies for reducing polypeptide levels. In some embodiments, the present disclosure provides technologies for targeting a polypeptide for degradation. In some embodiments, provided technologies comprise modulating interactions of polypeptides. In some embodiments, provided technologies comprise contacting a polypeptide with an agent, wherein a complex forms comprising the polypeptide, the agent, and another polypeptide. In some embodiments, the present disclosure provides technologies for forming a complex, comprising contacting an agent of present disclosure with polypeptides. In some embodiments, the present disclosure provides complexes comprising an agent of present disclosure and two polypeptides.

In some embodiments, an agent modulate polypeptide interactions as described herein. In some embodiments, two polypeptides interact with each other absence of the agent. In some embodiments, an agent provides new interactions between the polypeptides (which interactions does not exist absence of an agent). In some embodiments, an agent enhances an interaction between the polypeptides. In some embodiments, an agent reduces an interaction between the polypeptides. In some embodiments, the polypeptides do not interact with each absence of the agent. As those skilled in the art appreciates, polypeptides may interaction each other through residues, domains, surfaces, etc. In some embodiments, an agent binds to polypeptides simultaneously. In some embodiments, an agent binds to polypeptides cooperatively. In some embodiments, an agent interacts with one polypeptide through a set of residues and interacts with another polypeptide through another set of residues. In some embodiments, an agent binds to a polypeptide at a reduced level or weaker, or does not bind, compared to in the presence of another polypeptide.

In some embodiments, provided technologies modulate interactions, including inducing new interactions, between various target polypeptides with polypeptides associated with polypeptide degradation, e.g., E3 ligase. In some embodiments, a polypeptide is or comprises an E3 ligase. In some embodiments, an E3 ligase is or comprises an RBR E3 ligase. In some embodiments, an E3 ligase is or comprises a HECT E3 ligase. In some embodiments, an E3 ligase is or comprises an E3 ligase of a Cullin-RING (CRL) multi-subunit E3 family. In some embodiments, an E3 ligase is or comprises an E3 ligase of a RING/U-Box family. In some embodiments, an E3 ligase is or comprises an RBR E3 ligase. In some embodiments, an E3 ligase is or comprises RNF31. In some embodiments, an E3 ligase is or comprises WWP1. In some embodiments, an E3 ligase is or comprises WWP2. In some embodiments, an E3 ligase is or comprises WWP1^{WW-HECT}. In some embodiments, an E3 ligase is or comprises WWP1^{HECT}. In some embodiments, an E3 ligase is or comprises WWP2^{HECT}. In some embodiments, an E3 ligase is or comprises STUB1. In some embodiments, an E3 ligase is or comprises CUL1. In some embodiments, an E3 ligase is or comprises CUL2. In some embodiments, an E3 ligase is or comprises CUL5. In some embodiments, an E3 ligase is or comprises CRBN. In some embodiments, an E3 ligase is or comprises BIRC2. In some embodiments, an E3 ligase is or comprises FBXW7. In some embodiments, an E3 ligase is or comprises VHL. In some embodiments, an E3 ligase is or comprises XIAP. In some embodiments, an E3 ligase is or comprises MDM2. In some embodiments, an E3 ligase is or comprises a NEDD4-like E3 ligase.

Various target of interests may be targeted by provided technologies. In some embodiments, a target of interest is or comprises a polypeptide. In some embodiments, a target polypeptide is or comprises an enzyme. In some embodiments, a target peptide is or comprises transcription factor. In some embodiments, a target peptide is or comprises transcription coactivator. In some embodiments, a target polypeptide is PPIA. In some embodiments, a target polypeptide is TEAD4. In some embodiments, a target polypeptide is beta-catenin.

In some embodiments, the present disclosure provides technologies, e.g., agents and uses thereof, that can modulate polypeptide interactions. In some embodiments, provided technologies can enhance or induce new polypeptide interactions between a target of interest and another polypeptide. In some embodiments, a target of interest is or comprises a polypeptide. In some embodiments, an another polypeptide is or comprises an E3 ligase. In some embodiments, provided technologies can enhance or induce new interactions of a polypeptide with a target of interest, wherein the polypeptide is or comprises an E3 ligase. In some embodiments, the present disclosure provides complexes comprising an agent as described herein, a polypeptide that is or comprises an E3 ligase, and a target polypeptide. Among other things, provided technologies are useful for degradation of polypeptides. Certain technologies are presented below as examples.

### CHIP Trimerizers

In some embodiments, an E3 ligase is or comprises CHIP. In some embodiments, an E3 ligase is or comprises CHIP²³⁻³⁰³. In some embodiments, the present disclosure provides agents that can form complexes with CHIP and a target polypeptide. In some embodiments, the present disclosure provides agents that can form complexes with CHIP²³-3⁰³ and a target polypeptide.

### Cluster C86

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C86 as described herein. In some embodiments, an agent comprises X⁸⁶ X−¹X⁰X¹X²X³X⁴X⁵X⁶X⁷X⁸X⁹X¹⁰X¹¹X¹²X¹³X¹⁴X¹⁵X¹⁶X¹⁷, wherein each of X⁸⁶, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁸⁶X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁸⁶, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁵ comprises a side chain comprising a polar group;
X⁶ comprises a hydrophobic side chain;
X⁹ comprises a side chain comprising a polar group;
X¹⁰ comprises a hydrophobic side chain;
X¹² comprises a hydrophobic side chain;
X¹³ comprises a hydrophobic side chain; and
X¹⁴ comprises a side chain comprising an acidic group.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

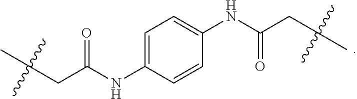 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising —OH. In some embodiments, X⁵ is S. In some embodiments, X⁵ is T.

Various amino acid residues may be utilized for X⁶. For example, in some embodiments, the side chain of X⁶ is aliphatic. In some embodiments, the side chain of X⁶ is C₁₋₆ alkyl. In some embodiments, X⁶ is A. In some embodiments, X⁶ is V. In some embodiments, X⁶ is I.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. For example, in some embodiments, X⁹ comprises a side chain comprising —OH. In some embodiments, X⁹ is S. In some embodiments, X⁹ is T.

Various amino acid residues may be utilized for X¹⁰. In some embodiments, X¹⁰ is I.

Various amino acid residues may be utilized for X¹². In some embodiments, X¹² is M. In some embodiments, X¹² is W.

Various amino acid residues may be utilized for X¹³. In some embodiments, X¹³ is V. In some embodiments, X¹³ is I.

Various amino acid residues may be utilized for X¹⁴. In some embodiments, X¹⁴ is D.

Various amino acid residues may be utilized for X¹⁵. In some embodiments, X¹⁵ is G.

Various amino acid residues may be utilized for X¹⁷. For example, in some embodiments, X¹⁷ comprises a side chain comprising a polar group. In some embodiments, a cluster is enriched for amino acid residues whose side chains comprise a polar group at X¹⁷. In some embodiments, X¹⁷ comprises a side chain comprising —OH. In some embodiments, X¹⁷ is S. In some embodiments, X¹⁷ is T. In some embodiments, the side chain of X¹⁷ is hydrophobic. In some embodiments, a cluster is enriched for amino acid residues comprising a hydrophobic side chain at X¹⁷. In some embodiments, the side chain of X¹⁷ is aliphatic. In some embodiments, the side chain of X¹⁷ is C₁₋₆ alkyl. In some embodiments, X¹⁷ is A. In some embodiments, X¹⁷ is V. In some embodiments, X¹⁷ is I. In some embodiments, X¹⁷ is L.

In some embodiments, an agent comprising a cluster C86 sequence is capable of binding to CHIP. In some embodiments, an agent comprising a cluster C86 sequence is capable of promoting an interaction between CHIP and PPIA. In some embodiments, an agent comprising a cluster C86 sequence is capable of binding to PPIA in the presence of CHIP.

In some embodiments, the present disclosure provides a complex comprising PPIA, CHIP and an agent of cluster C86. In some embodiments, the present disclosure provides methods for reducing levels of PPIA in a system, comprising administering or delivering to the system an effective amount of an agent of cluster C86. In some embodiments, the present disclosure provides a method for preventing or treating a condition, disorder or disease associated with PPIA, comprising administering or delivering to a subject suffering therefrom an effective amount of an agent of cluster C86.

### Cluster C87

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C87 as described herein. In some embodiments, an agent comprises X⁸⁷X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁸⁷, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁸⁷X⁻¹X⁰X¹X²X³X⁴X⁵X⁶X⁷X⁸X⁹X¹⁰X¹¹X¹²X³X⁴X¹⁵X¹⁶X¹⁷,

wherein:
each of X⁸⁷, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁰ comprises a hydrophobic side chain or a side chain comprising an aromatic group;
X¹ comprises a hydrophobic side chain or a side chain comprising an aromatic group;
X⁹ comprises a hydrophobic side chain.

Various amino acid residues may be utilized for X⁸⁷. For example, in some embodiments, X⁸⁷ comprises a side chain comprising a polar group. In some embodiments, a cluster is enriched for amino acid residues whose side chains comprise a polar group at X⁸⁷. In some embodiments, X⁸⁷ comprises a side chain comprising —OH. In some embodiments, X⁸⁷ is T. In some embodiments, the side chain of X⁸⁷ is hydrophobic. In some embodiments, a cluster is enriched for amino acid residues comprising a hydrophobic side chain at X⁸⁷. In some embodiments, the side chain of X⁸⁷ is aliphatic. In some embodiments, the side chain of X⁸⁷ is C₁₋₅ alkyl. In some embodiments, X⁸⁷ is V. In some embodiments, X⁸⁷ is I.

Various amino acid residues may be utilized for X⁻¹. For example, in some embodiments, X-1 is P.

Various amino acid residues may be utilized for X⁰. In some embodiments, X⁰ comprises a side chain comprising an aromatic group. In some embodiments, X⁰ is F. In some embodiments, X⁰ is Y.

Various amino acid residues may be utilized for X¹. In some embodiments, X¹ comprises a side chain comprising an aromatic group. For example, in some embodiments, X¹ is F. In some embodiments, X¹ is Y.

Various amino acid residues may be utilized for X². For example, in some embodiments, the side chain of X² is hydrophobic. In some embodiments, a cluster is enriched for amino acid residues comprising a hydrophobic side chain at X². In some embodiments, X² comprises a side chain comprising an aromatic group. In some embodiments, X² is W.

Various amino acid residues may be utilized for X³. For example, in some embodiments, X³ comprises a side chain comprising an acidic group. In some embodiments, a cluster is enriched for amino acid residues whose side chains comprise an acidic group at X³. In some embodiments, X³ comprises a side chain comprising —COOH. In some embodiments, X³ is D. In some embodiments, X³ is E. In some embodiments, X³ is P.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

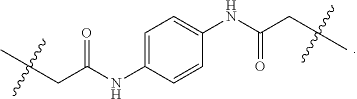 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising a polar group. In some embodiments, a cluster is enriched for amino acid residues whose side chains comprise a polar group at X⁵. In some embodiments, X⁵ comprises a side chain comprising —C(O)NH₂. In some embodiments, X⁵ is Q.

Various amino acid residues may be utilized for X⁶. For example, in some embodiments, the side chain of X⁶ is hydrophobic. In some embodiments, a cluster is enriched for amino acid residues comprising a hydrophobic side chain at X⁶. In some embodiments, X⁶ comprises a side chain comprising an aromatic group. In some embodiments, X⁶ is H. In some embodiments, X⁶ is F. In some embodiments, X⁶ is Y.

Various amino acid residues may be utilized for X⁷. In some embodiments, X⁷ is A.

Various amino acid residues may be utilized for X⁸. In some embodiments, the side chain of X⁸ is aliphatic. In some embodiments, the side chain of X⁸ is C₁₋₆ alkyl. In some embodiments, X⁸ is A.

Various amino acid residues may be utilized for X⁹. For example, in some embodiments, the side chain of X⁹ is aliphatic. In some embodiments, the side chain of X⁹ is C₁₋₆ alkyl. In some embodiments, X⁹ is A. In some embodiments, X⁹ is I.

Various amino acid residues may be utilized for X¹⁰. For example, in some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —OH. In some embodiments, X¹⁰ is T. In some embodiments, the side chain of X¹⁰ is hydrophobic. In some embodiments, a cluster is enriched for amino acid residues comprising a hydrophobic side chain at X¹⁰. In some embodiments, X¹⁰ is M.

In some embodiments, an agent comprising a cluster C87 sequence is capable of binding to CHIP. In some embodiments, an agent comprising a cluster C87 sequence is capable of promoting an interaction between CHIP and TEAD4. In some embodiments, an agent comprising a cluster C87 sequence is capable of binding to TEAD4 in the presence of CHIP.

In some embodiments, the present disclosure provides a complex comprising TEAD4, CHIP and an agent of C87. In some embodiments, the present disclosure provides methods for reducing levels of TEAD4 in a system, comprising administering or delivering to the system an effective amount of an agent of C87. In some embodiments, the present disclosure provides a method for preventing or treating a condition, disorder or disease associated with TEAD4, comprising administering or delivering to a subject suffering therefrom an effective amount of an agent of C87.

### Cluster C88

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C88 as described herein. In some embodiments, an agent comprises X⁸⁸X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁸⁸, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising: X⁸⁸X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁸⁸, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X² comprises a hydrophobic side chain or a side chain comprising an aromatic group;
X³ comprises a side chain comprising a polar group;
X⁵ comprises a hydrophobic side chain;
X⁶ comprises a side chain comprising an acidic group;
X⁹ comprises a hydrophobic side chain or a side chain comprising an aromatic group;
X¹⁰ comprises a hydrophobic side chain;
X¹² comprises a side chain comprising an acidic group;
X¹³ comprises a side chain comprising a polar group;
X¹⁴ comprises a hydrophobic side chain or a side chain comprising an aromatic group; and
X¹⁵ comprises a hydrophobic side chain.

Various amino acid residues may be utilized for X². In some embodiments, X² comprises a side chain comprising an aromatic group. In some embodiments, X² is W.

Various amino acid residues may be utilized for X³. In some embodiments, X³ comprises a side chain comprising a polar group. In some embodiments, X³ comprises a side chain comprising —OH. In some embodiments, X³ is S.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

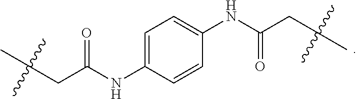 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, the side chain of X⁵ is aliphatic. In some embodiments, the side chain of X⁵ is C₁₋₆ alkyl. In some embodiments, X⁵ is A. In some embodiments, X⁵ is V.

Various amino acid residues may be utilized for X⁶. In some embodiments, X⁶ is E.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising an aromatic group. In some embodiments, X⁹ is Y. In some embodiments, X⁹ is F.

Various amino acid residues may be utilized for X¹⁰. In some embodiments, the side chain of X¹⁰ is hydrophobic. In some embodiments, a cluster is enriched for amino acid residues comprising a hydrophobic side chain at X¹⁰. In some embodiments, X¹⁰ is M. In some embodiments, the side chain of X¹⁰ is aliphatic. In some embodiments, the side chain of X¹⁰ is C₁₋₆ alkyl. In some embodiments, X¹⁰ is I. In some embodiments, X¹⁰ is L.

Various amino acid residues may be utilized for X¹². In some embodiments, X¹² is E.

Various amino acid residues may be utilized for X¹³. In some embodiments, X¹³ comprises a side chain comprising —C(O)NH₂. In some embodiments, X¹³ is N. In some embodiments, X¹³ is Q.

Various amino acid residues may be utilized for X¹⁴. In some embodiments, X¹⁴ comprises a side chain comprising an aromatic group. In some embodiments, X¹⁴ is H. In some embodiments, X¹⁴ is Y.

Various amino acid residues may be utilized for X¹⁵. In some embodiments, the side chain of X¹⁵ is aliphatic. In some embodiments, the side chain of X¹⁵ is C₁₋₆ alkyl. In some embodiments, X¹⁵ is V.

Various amino acid residues may be utilized for X¹⁷. For example, in some embodiments, X¹⁷ comprises a side chain comprising a polar group. In some embodiments, a cluster is enriched for amino acid residues whose side chains comprise a polar group at X¹⁷. In some embodiments, X¹⁷ comprises a side chain comprising —OH. In some embodiments, X¹⁷ is S. In some embodiments, X¹⁷ is T. In some embodiments, the side chain of X¹⁷ is hydrophobic. In some embodiments, a cluster is enriched for amino acid residues comprising a hydrophobic side chain at X¹⁷. In some embodiments, the side chain of X¹⁷ is aliphatic. In some embodiments, the side chain of X¹⁷ is C₁₋₆ alkyl. In some embodiments, X¹⁷ is A. In some embodiments, X¹⁷ is V. In some embodiments, X¹⁷ is I.

In some embodiments, an agent comprising a cluster C88 sequence is capable of binding to CHIP. In some embodiments, an agent comprising a cluster C88 sequence is capable of promoting an interaction between CHIP and PPIA. In some embodiments, an agent comprising a cluster C88 sequence is capable of binding to PPIA in the presence of CHIP.

In some embodiments, the present disclosure provides a complex comprising PPIA, CHIP and an agent of cluster C88. In some embodiments, the present disclosure provides methods for reducing levels of PPIA in a system, comprising administering or delivering to the system an effective amount of an agent of cluster C88. In some embodiments, the present disclosure provides a method for preventing or treating a condition, disorder or disease associated with PPIA, comprising administering or delivering to a subject suffering therefrom an effective amount of an agent of cluster C88.

### Cluster C89

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C89 as described herein. In some embodiments, an agent comprises X⁸⁹X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁸⁹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising: X⁸⁹X⁻¹X⁰X¹X²X³X⁴X⁵X⁶X⁷X⁸X⁹X¹¹X¹²X¹³X¹⁴X¹⁵X¹⁶X¹⁷,

wherein:
each of X⁸⁹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁹ comprises a side chain comprising a polar group;
X¹⁰ comprises a hydrophobic side chain;
X¹² comprises a hydrophobic side chain;
X¹³ comprises a hydrophobic side chain; and
X¹⁴ comprises a side chain comprising an acidic group.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

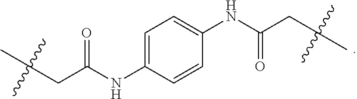 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ is G.

Various amino acid residues may be utilized for X⁶. In some embodiments, X⁶ is P. In some embodiments, a cluster is enriched for P at X⁶.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, a cluster is enriched for amino acid residues whose side chains comprise —OH at X⁹. In some embodiments, X⁹ comprises a side chain comprising —OH. In some embodiments, X⁹ is S. In some embodiments, X⁹ is T.

Various amino acid residues may be utilized for X¹⁰. In some embodiments, the side chain of X¹³ is aliphatic. In some embodiments, the side chain of X¹³ is C₁₋₆ alkyl. In some embodiments, X¹³ is I.

Various amino acid residues may be utilized for X¹². For example, in some embodiments, X¹² comprises a side chain comprising an aromatic group. In some embodiments, X¹² is W.

Various amino acid residues may be utilized for X¹³. In some embodiments, the side chain of X¹³ is aliphatic. In some embodiments, the side chain of X¹³ is C₁₋₆ alkyl. In some embodiments, X¹³ is V. In some embodiments, X¹³ is I.

Various amino acid residues may be utilized for X¹⁴. In some embodiments, X¹⁴ comprises a side chain comprising —COOH. In some embodiments, X¹⁴ is D.

Various amino acid residues may be utilized for X¹⁵. In some embodiments, X¹⁵ is G.

Various amino acid residues may be utilized for X¹⁶. For example, in some embodiments, the side chain of X¹⁶ is hydrophobic. In some embodiments, a cluster is enriched for amino acid residues comprising a hydrophobic side chain at X¹⁶. In some embodiments, X¹⁶ is M. In some embodiments, the side chain of X¹⁶ is aliphatic. In some embodiments, the side chain of X¹⁶ is C₁₋₆ alkyl. In some embodiments, X¹⁶ is V. In some embodiments, X¹⁶ is I. In some embodiments, X¹⁶ comprises a side chain comprising an aromatic group. In some embodiments, X¹⁶ is F. In some embodiments, X¹⁶ is Y.

Various amino acid residues may be utilized for X¹⁷. For example, in some embodiments, the side chain of X¹⁷ is hydrophobic. In some embodiments, a cluster is enriched for amino acid residues comprising a hydrophobic side chain at X¹⁷. In some embodiments, the side chain of X¹⁷ is aliphatic. In some embodiments, the side chain of X¹⁷ is C₁₋₆ alkyl. In some embodiments, X¹⁷ is V. In some embodiments, X¹⁷ is I.

In some embodiments, an agent comprising a cluster C89 sequence is capable of binding to CHIP. In some embodiments, an agent comprising a cluster C89 sequence is capable of promoting an interaction between CHIP and PPIA. In some embodiments, an agent comprising a cluster C89 sequence is capable of binding to PPIA in the presence of CHIP.

In some embodiments, the present disclosure provides a complex comprising PPIA, CHIP and an agent of cluster C89. In some embodiments, the present disclosure provides methods for reducing levels of PPIA in a system, comprising administering or delivering to the system an effective amount of an agent of cluster C89. In some embodiments, the present disclosure provides a method for preventing or treating a condition, disorder or disease associated with PPIA, comprising administering or delivering to a subject suffering therefrom an effective amount of an agent of cluster C89.

### Cluster C90

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C90 as described herein. In some embodiments, an agent comprises X⁹⁰X⁻¹X⁰X¹X²X³X⁴X⁵X⁶X⁷X⁸X⁹X⁰X¹X¹²X¹³X¹⁴X¹⁵X¹⁶X¹⁷, wherein each of X⁹⁰, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁹⁰X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁹⁰, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X¹ comprises a hydrophobic side chain;
X² comprises a side chain comprising an acidic group;
X³ comprises a hydrophobic side chain;
X⁶ comprises a side chain comprising an aromatic group;
X⁹ comprises a side chain comprising an acidic group;
X¹⁰ comprises a hydrophobic side chain;
X¹² comprises a hydrophobic side chain or a side chain comprising an aromatic group;
X¹³ comprises a hydrophobic side chain; and
X¹⁴ comprises a hydrophobic side chain or a side chain comprising an aromatic group.

Various amino acid residues may be utilized for X⁻¹. For example, in some embodiments, X⁻¹ comprises a side chain comprising an acidic group. In some embodiments, a cluster is enriched for amino acid residues whose side chains comprise an acidic group at X⁻¹. In some embodiments, X⁻¹ comprises a side chain comprising —COOH. In some embodiments, X⁻¹ is D. In some embodiments, X⁻¹ is E.

Various amino acid residues may be utilized for X⁰. For example, in some embodiments, X⁰ is P. In some embodiments, X⁰ is G. In some embodiments, X⁰ is E.

Various amino acid residues may be utilized for X¹. In some embodiments, X¹ is M. In some embodiments, X¹ is F. In some embodiments, the side chain of X¹ is aliphatic. In some embodiments, the side chain of X¹ is C₁₋₆ alkyl. In some embodiments, X¹ is V. In some embodiments, X¹ is I.

Various amino acid residues may be utilized for X². In some embodiments, X² is D.

Various amino acid residues may be utilized for X³. In some embodiments, X³ is M. In some embodiments, the side chain of X³ is aliphatic. In some embodiments, the side chain of X³ is C₁₋₆ alkyl. In some embodiments, X³ is V. In some embodiments, X³ is I. In some embodiments, X³ is L.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

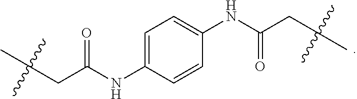 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, X⁵ comprises a side chain comprising an acidic group. In some embodiments, a cluster is enriched for amino acid residues whose side chains comprise an acidic group at X⁵. In some embodiments, X⁵ comprises a side chain comprising —COOH. In some embodiments, X⁵ is D. In some embodiments, X⁵ is E.

Various amino acid residues may be utilized for X⁶. In some embodiments, X⁶ is H. In some embodiments, X⁶ is F. In some embodiments, X⁶ is Y.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising —COOH. In some embodiments, X⁹ is D. In some embodiments, X⁹ is E.

Various amino acid residues may be utilized for X¹⁰. In some embodiments, X¹⁰ is M. In some embodiments, the side chain of X¹⁰ is aliphatic. In some embodiments, the side chain of X¹ is C₁₋₆ alkyl. In some embodiments, X¹⁰ is V. In some embodiments, X¹⁰ is I.

Various amino acid residues may be utilized for X¹². In some embodiments, X¹² is H. In some embodiments, X¹² is W.

Various amino acid residues may be utilized for X¹³. In some embodiments, X¹³ comprises a side chain comprising an aromatic group. In some embodiments, X¹³ is W. In some embodiments, the side chain of X¹³ is aliphatic. In some embodiments, the side chain of X¹³ is C₁₋₆ alkyl. In some embodiments, X¹³ is L. In some embodiments, X¹³ is I.

Various amino acid residues may be utilized for X¹⁴. In some embodiments, X¹⁴ is L. In some embodiments, X¹⁴ comprises a side chain comprising an aromatic group. In some embodiments, X¹⁴ is Y. In some embodiments, X¹⁴ is W.

Various amino acid residues may be utilized for X¹⁵. In some embodiments, X¹⁵ comprises a hydrophobic side chain. In some embodiments, X¹⁵ is M. In some embodiments, the side chain of X¹⁵ is aliphatic. In some embodiments, the side chain of X¹⁵ is C₁₋₆ alkyl. In some embodiments, X¹⁵ is A. In some embodiments, X¹⁵ is V. In some embodiments, X¹⁵ is I. In some embodiments, X¹⁵ is L.

Various amino acid residues may be utilized for X¹⁶. For example, in some embodiments, X¹⁶ comprises a side chain comprising an acidic group. In some embodiments, a cluster is enriched for amino acid residues whose side chains comprise an acidic group at X¹⁶. In some embodiments, X¹⁶ comprises a side chain comprising —COOH. In some embodiments, X¹⁶ is D. In some embodiments, X¹⁶ is E.

In some embodiments, an agent comprising a cluster C90 sequence is capable of binding to CHIP. In some embodiments, an agent comprising a cluster C90 sequence is capable of promoting an interaction between CHIP and TEAD4. In some embodiments, an agent comprising a cluster C90 sequence is capable of binding to TEAD4 in the presence of CHIP.

In some embodiments, the present disclosure provides a complex comprising TEAD4, CHIP and an agent of C90. In some embodiments, the present disclosure provides methods for reducing levels of TEAD4 in a system, comprising administering or delivering to the system an effective amount of an agent of C90. In some embodiments, the present disclosure provides a method for preventing or treating a condition, disorder or disease associated with TEAD4, comprising administering or delivering to a subject suffering therefrom an effective amount of an agent of C90.

### Cluster C94

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C94 as described herein. In some embodiments, an agent comprises X⁹⁴X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁹⁴, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁹⁴X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁹⁴, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁹ comprises a side chain comprising an acid group;
X¹⁰ comprises a hydrophobic side chain;
X¹² comprises a hydrophobic side chain or a side chain comprising an aromatic group;
X¹³ comprises a hydrophobic side chain;
X¹⁴ comprises a hydrophobic side chain or a side chain comprising an aromatic group;
X¹⁵ comprises a hydrophobic side chain or a side chain comprising an aromatic group;
X¹⁶ comprises a hydrophobic side chain; and
X¹⁷ comprises a hydrophobic side chain or a side chain comprising an aromatic group.

Various amino acid residues may be utilized for X². In some embodiments, X² comprises a side chain comprising a polar group. In some embodiments, a cluster is enriched for amino acid residues whose side chains comprise a polar group at X². In some embodiments, X² comprises a side chain comprising —OH. In some embodiments, X² is S. In some embodiments, X² is T. In some embodiments, X² comprises a side chain comprising —C(O)NH₂. In some embodiments, X² is N. In some embodiments, X² is Q.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

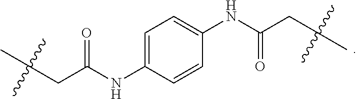 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. For example, in some embodiments, the side chain of X⁵ is hydrophobic. In some embodiments, a cluster is enriched for amino acid residues comprising a hydrophobic side chain at X⁵. In some embodiments, the side chain of X⁵ is aliphatic. In some embodiments, the side chain of X⁵ is C₁₋₆ alkyl. In some embodiments, X⁵ is A. In some embodiments, X⁵ is V. In some embodiments, X⁵ is I. In some embodiments, X⁵ is L. In some embodiments, X⁵ is P.

Various amino acid residues may be utilized for X⁶. For example, in some embodiments, X⁶ comprises a side chain comprising a basic group. In some embodiments, a cluster is enriched for amino acid residues whose side chains comprise a basic group at X⁶. In some embodiments, X⁶ is H. In some embodiments, X⁶ comprises a side chain comprising an acidic group. In some embodiments, a cluster is enriched for amino acid residues whose side chains comprise an acidic group at X⁶. In some embodiments, X⁶ comprises a side chain comprising —COOH. In some embodiments, X⁶ is D. In some embodiments, X⁶ is E. In some embodiments, X⁶ comprises a side chain comprising a polar group. In some embodiments, a cluster is enriched for amino acid residues whose side chains comprise a polar group at X⁶. In some embodiments, X⁶ comprises a side chain comprising —C(O)NH₂. In some embodiments, X⁶ is N. In some embodiments, X⁶ is Q.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising —COOH. In some embodiments, X⁹ is D. In some embodiments, X⁹ is E.

Various amino acid residues may be utilized for X¹⁰. In some embodiments, the side chain of X¹⁰ is aliphatic. In some embodiments, the side chain of X¹⁰ is C₁₋₆ alkyl. In some embodiments, X¹⁰ is V. In some embodiments, X¹⁰ is I.

Various amino acid residues may be utilized for X¹². In some embodiments, X¹² is W.

Various amino acid residues may be utilized for X¹³. In some embodiments, the side chain of X¹³ is aliphatic. In some embodiments, the side chain of X¹³ is C₁₋₆ alkyl. In some embodiments, X¹³ is I. In some embodiments, X¹³ is L.

Various amino acid residues may be utilized for X¹⁴. In some embodiments, X¹⁴ is Y.

Various amino acid residues may be utilized for X¹⁵. In some embodiments, X¹⁵ is W.

Various amino acid residues may be utilized for X¹⁶. In some embodiments, the side chain of X¹⁶ is aliphatic. In some embodiments, the side chain of X¹⁶ is C₁₋₆ alkyl. In some embodiments, X¹⁶ is V. In some embodiments, X¹⁶ is L.

Various amino acid residues may be utilized for X¹⁷. In some embodiments, X¹⁷ is H. In some embodiments, X¹⁷ is Y.

In some embodiments, an agent comprising a cluster C94 sequence is capable of binding to CHIP. In some embodiments, an agent comprising a cluster C94 sequence is capable of promoting an interaction between CHIP and PPIA. In some embodiments, an agent comprising a cluster C94 sequence is capable of binding to PPIA in the presence of CHIP.

In some embodiments, the present disclosure provides a complex comprising PPIA, CHIP and an agent of cluster C94. In some embodiments, the present disclosure provides methods for reducing levels of PPIA in a system, comprising administering or delivering to the system an effective amount of an agent of cluster C94. In some embodiments, the present disclosure provides a method for preventing or treating a condition, disorder or disease associated with PPIA, comprising administering or delivering to a subject suffering therefrom an effective amount of an agent of cluster C94.

### MDM2 Trimerizers

In some embodiments, an E3 ligase is or comprises MDM2. In some embodiments, an E3 ligase is or comprises MDM2²⁵⁻¹⁰⁹. In some embodiments, the present disclosure provides agents that can form complexes with MDM2 and a target polypeptide. In some embodiments, the present disclosure provides agents that can form complexes with MDM2²⁵⁻¹⁰⁹ and a target polypeptide.

### Cluster C91

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C91 as described herein. In some embodiments, an agent comprises X⁹¹X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁹¹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁹¹X⁻¹X⁰X¹X²X³X⁴X⁵X⁶X⁷X⁸X⁹X¹¹X¹²X¹³X¹⁴X¹⁵X¹⁶X¹⁷,

wherein:
each of X⁹¹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X³ comprises a hydrophobic side chain;
X⁵ comprises a hydrophobic side chain or a side chain comprising an aromatic group;
X⁶ comprises a side chain comprising a polar group;
X⁹ comprises a hydrophobic side chain or a side chain comprising an aromatic group;
X¹² comprises a hydrophobic side chain;
X¹⁴ comprises a side chain comprising an acidic group;
X¹⁵ comprises a side chain comprising an acidic group; and
X¹⁶ comprises a hydrophobic side chain or a side chain comprising an aromatic group.

Various amino acid residues may be utilized for X⁹¹. In some embodiments, the side chain of X⁹¹ is hydrophobic. In some embodiments, a cluster is enriched for amino acid residues comprising a hydrophobic side chain at X⁹¹. In some embodiments, X⁹¹ is M. In some embodiments, the side chain of X⁹¹ is aliphatic. In some embodiments, the side chain of X⁹¹ is C₁₋₆ alkyl. In some embodiments, X⁹¹ is V. In some embodiments, X⁹¹ is I. In some embodiments, X⁹¹ comprises a side chain comprising an aromatic group. In some embodiments, X⁹¹ is F. In some embodiments, X⁹¹ is Y. In some embodiments, X⁹¹ is W.

Various amino acid residues may be utilized for X¹. For example, in some embodiments, X¹ comprises a side chain comprising an acidic group. In some embodiments, a cluster is enriched for amino acid residues whose side chains comprise an acidic group at X¹. In some embodiments, X¹ comprises a side chain comprising —COOH. In some embodiments, X¹ is E. In some embodiments, X¹ comprises a side chain comprising a polar group. In some embodiments, a cluster is enriched for amino acid residues whose side chains comprise a polar group at X¹. In some embodiments, X¹ comprises a side chain comprising —C(O)NH₂. In some embodiments, X¹ is Q.

Various amino acid residues may be utilized for X³. In some embodiments, the side chain of X³ is aliphatic. In some embodiments, the side chain of X³ is C₁₋₆ alkyl. In some embodiments, X³ is V. In some embodiments, X³ is I.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

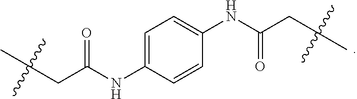 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ comprises a side chain comprising an aromatic group. In some embodiments, X⁵ is F. In some embodiments, X⁵ is Y.

Various amino acid residues may be utilized for X⁶. In some embodiments, X⁶ comprises a side chain comprising —C(O)NH₂. In some embodiments, X⁶ is Q.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising an aromatic group. In some embodiments, X⁹ is W.

Various amino acid residues may be utilized for X¹⁰. In some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —OH. In some embodiments, X¹⁰ is T. In some embodiments, X¹⁰ comprises a side chain comprising —C(O)NH₂. In some embodiments, X¹⁰ is Q. In some embodiments, the side chain of X¹⁰ is hydrophobic. In some embodiments, a cluster is enriched for amino acid residues comprising a hydrophobic side chain at X¹⁰. In some embodiments, X¹⁰ is M. In some embodiments, the side chain of X¹⁰ is aliphatic. In some embodiments, the side chain of X¹⁰ is C₁₋₆ alkyl. In some embodiments, X¹⁰ is L. In some embodiments, X¹⁰ comprises a side chain comprising an aromatic group. In some embodiments, X¹⁰ is Y.

Various amino acid residues may be utilized for X¹². In some embodiments, X¹² comprises a side chain comprising an aromatic group. In some embodiments, X¹² is F. In some embodiments, the side chain of X¹² is aliphatic. In some embodiments, the side chain of X¹² is C₁₋₆ alkyl. In some embodiments, X¹² is V. In some embodiments, X¹² is L.

Various amino acid residues may be utilized for X¹³. In some embodiments, X¹³ comprises a side chain comprising a polar group. In some embodiments, a cluster is enriched for amino acid residues whose side chains comprise a polar group at X¹³. In some embodiments, X¹³ comprises a side chain comprising —OH. In some embodiments, X¹³ is S. In some embodiments, X¹³ is T.

Various amino acid residues may be utilized for X¹⁴. In some embodiments, X¹⁴ is L. In some embodiments, X¹⁴ comprises a side chain comprising an aromatic group. In some embodiments, X¹⁴ is Y. In some embodiments, X¹⁴ is W.

Various amino acid residues may be utilized for X¹⁵. In some embodiments, X¹⁴ comprises a side chain comprising —COOH. In some embodiments, X¹⁴ is D. In some embodiments, X¹⁴ is E.

Various amino acid residues may be utilized for X¹⁵. In some embodiments, X¹⁵ comprises a side chain comprising —COOH. In some embodiments, X¹⁵ is D. In some embodiments, X¹⁵ is E.

Various amino acid residues may be utilized for X¹⁶. In some embodiments, X¹⁶ comprises a side chain comprising an aromatic group. In some embodiments, X¹⁶ is Y. In some embodiments, X¹⁶ is W.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of MDM2 or amino acid residues corresponding thereto: Thr26, Met50, Leu54, Leu57, Gly58, Ile61, Met62, Tyr67, Gln72, His73, Val75, Val93, Lys94, His96, Ile99, Tyr100, and Tyr104. In some embodiments, it interacts with Thr26 of MDM2 or an amino acid residue corresponding thereto. In some embodiments, it interacts with Met50 of MDM2 or an amino acid residue corresponding thereto. In some embodiments, it interacts with Leu54 of MDM2 or an amino acid residue corresponding thereto. In some embodiments, it interacts with Leu57 of MDM2 or an amino acid residue corresponding thereto. In some embodiments, it interacts with Gly58 of MDM2 or an amino acid residue corresponding thereto. In some embodiments, it interacts with Ile61 of MDM2 or an amino acid residue corresponding thereto. In some embodiments, it interacts with Met62 of MDM2 or an amino acid residue corresponding thereto. In some embodiments, it interacts with Tyr67 of MDM2 or an amino acid residue corresponding thereto. In some embodiments, it interacts with Gln72 of MDM2 or an amino acid residue corresponding thereto. In some embodiments, it interacts with His73 of MDM2 or an amino acid residue corresponding thereto. In some embodiments, it interacts with Val75 of MDM2 or an amino acid residue corresponding thereto. In some embodiments, it interacts with Val93 of MDM2 or an amino acid residue corresponding thereto. In some embodiments, it interacts with Lys94 of MDM2 or an amino acid residue corresponding thereto. In some embodiments, it interacts with His96 of MDM2 or an amino acid residue corresponding thereto. In some embodiments, it interacts with Ile99 of MDM2 or an amino acid residue corresponding thereto. In some embodiments, it interacts with Tyr100 of MDM2 or an amino acid residue corresponding thereto. In some embodiments, it interacts with Tyr104 of MDM2 or an amino acid residue corresponding thereto. In some embodiments, an agent described herein, e.g., a stapled peptide, interacts with one or more (e.g., 1, 2, 3, 4, 5, 6, 7, 8, 9, 10, 11, 12, 13, 14, 15, 16, 17, or 18) or all of the following amino acid residues of beta-catenin or amino acid residues corresponding thereto: Tyr432, Arg474, His475, Arg515, Leu519, His578, Arg582, Arg612, Cys619, Glu620, Gln623, Gly650, Thr653, Tyr654, Ala656, Ala657, Phe660, and Arg661. In some embodiments, it interacts with Tyr432 of beta-catenin or an amino acid residue corresponding thereto. In some embodiments, it interacts with Arg474 of beta-catenin or an amino acid residue corresponding thereto. In some embodiments, it interacts with His475 of beta-catenin or an amino acid residue corresponding thereto. In some embodiments, it interacts with Arg515 of beta-catenin or an amino acid residue corresponding thereto. In some embodiments, it interacts with Leu519 of beta-catenin or an amino acid residue corresponding thereto. In some embodiments, it interacts with His578 of beta-catenin or an amino acid residue corresponding thereto. In some embodiments, it interacts with Arg582 of beta-catenin or an amino acid residue corresponding thereto. In some embodiments, it interacts with Arg612 of beta-catenin or an amino acid residue corresponding thereto. In some embodiments, it interacts with Glu620 of beta-catenin or an amino acid residue corresponding thereto. In some embodiments, it interacts with Cys619 of beta-catenin or an amino acid residue corresponding thereto. In some embodiments, it interacts with Gln623 of beta-catenin or an amino acid residue corresponding thereto. In some embodiments, it interacts with Gly650 of beta-catenin or an amino acid residue corresponding thereto. In some embodiments, it interacts with Thr653 of beta-catenin or an amino acid residue corresponding thereto. In some embodiments, it interacts with Tyr654 of beta-catenin or an amino acid residue corresponding thereto. In some embodiments, it interacts with Ala656 of beta-catenin or an amino acid residue corresponding thereto. In some embodiments, it interacts with Ala657 of beta-catenin or an amino acid residue corresponding thereto. In some embodiments, it interacts with Phe660 of beta-catenin or an amino acid residue corresponding thereto. In some embodiments, it interacts with Phe661 of beta-catenin or an amino acid residue corresponding thereto. In some embodiments, in a complex comprising an agent, beta-catenin and MDM2, one or more of Asn430, Tyr432, Lys433, Arg474, His475, Gln476, Glu479 and Arg582 of beta-catenin or amino acid residues corresponding thereto interact with one or more of Glu25, Thr26, His96, Arg97, Tyr104, Val109 and Val110 of MDM2 or amino acid residues corresponding thereto.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of MDM2 or amino acid residues corresponding thereto: Thr26, Met50, Leu54, Leu57, Gly58, Ile61, Met62, Tyr67, Gln72, His73, Val75, Val93, Lys94, His96, Ile99, Tyr100, and Tyr104. In some embodiments, it interacts with Thr26 of MDM2 or an amino acid residue corresponding thereto. In some embodiments, it interacts with Met50 of MDM2 or an amino acid residue corresponding thereto. In some embodiments, it interacts with Leu54 of MDM2 or an amino acid residue corresponding thereto. In some embodiments, it interacts with Leu57 of MDM2 or an amino acid residue corresponding thereto. In some embodiments, it interacts with Gly58 of MDM2 or an amino acid residue corresponding thereto. In some embodiments, it interacts with Ile61 of MDM2 or an amino acid residue corresponding thereto. In some embodiments, it interacts with Met62 of MDM2 or an amino acid residue corresponding thereto. In some embodiments, it interacts with Tyr67 of MDM2 or an amino acid residue corresponding thereto. In some embodiments, it interacts with Gln72 of MDM2 or an amino acid residue corresponding thereto. In some embodiments, it interacts with His73 of MDM2 or an amino acid residue corresponding thereto. In some embodiments, it interacts with Val75 of MDM2 or an amino acid residue corresponding thereto. In some embodiments, it interacts with Val93 of MDM2 or an amino acid residue corresponding thereto. In some embodiments, it interacts with Lys94 of MDM2 or an amino acid residue corresponding thereto. In some embodiments, it interacts with His96 of MDM2 or an amino acid residue corresponding thereto. In some embodiments, it interacts with Ile99 of MDM2 or an amino acid residue corresponding thereto. In some embodiments, it interacts with Tyr100 of MDM2 or an amino acid residue corresponding thereto. In some embodiments, it interacts with Tyr104 of MDM2 or an amino acid residue corresponding thereto. In some embodiments, an agent described herein, e.g., a stapled peptide, interacts with one or more (e.g., 1, 2, 3, 4, 5, 6, 7, 8, 9, 10, 11, 12, 13, 14, or 15) or all of the following amino acid residues of beta-catenin or amino acid residues corresponding thereto: Arg474, His475, Arg515, Leu519, His578, Arg582, Arg612, Glu620, Gln623, Gly650, Thr653, Tyr654, Ala656, Ala657, and Phe660. In some embodiments, it interacts with Arg474 of beta-catenin or an amino acid residue corresponding thereto. In some embodiments, it interacts with His475 of beta-catenin or an amino acid residue corresponding thereto. In some embodiments, it interacts with Arg515 of beta-catenin or an amino acid residue corresponding thereto. In some embodiments, it interacts with Leu519 of beta-catenin or an amino acid residue corresponding thereto. In some embodiments, it interacts with His578 of beta-catenin or an amino acid residue corresponding thereto. In some embodiments, it interacts with Arg582 of beta-catenin or an amino acid residue corresponding thereto. In some embodiments, it interacts with Arg612 of beta-catenin or an amino acid residue corresponding thereto. In some embodiments, it interacts with Glu620 of beta-catenin or an amino acid residue corresponding thereto. In some embodiments, it interacts with Gln623 of beta-catenin or an amino acid residue corresponding thereto. In some embodiments, it interacts with Gly650 of beta-catenin or an amino acid residue corresponding thereto. In some embodiments, it interacts with Thr653 of beta-catenin or an amino acid residue corresponding thereto. In some embodiments, it interacts with Tyr654 of beta-catenin or an amino acid residue corresponding thereto. In some embodiments, it interacts with Ala656 of beta-catenin or an amino acid residue corresponding thereto. In some embodiments, it interacts with Ala657 of beta-catenin or an amino acid residue corresponding thereto. In some embodiments, it interacts with Phe660 of beta-catenin or an amino acid residue corresponding thereto. In some embodiments, in a complex comprising an agent, beta-catenin and MDM2, one or more of Tyr432, Arg474, His475, Gln476, Als478, Glu479, Arg582 and Glu649 of beta-catenin or amino acid residues corresponding thereto interact with one or more of Glu25, Lys51, His96, Arg97, Tyr104, and Val109 of MDM2 or amino acid residues corresponding thereto.

In some embodiments, an agent comprising a cluster C91 sequence is capable of binding to MDM2. In some embodiments, an agent comprising a cluster C91 sequence is capable of promoting an interaction between MDM2 and beta-catenin. In some embodiments, an agent comprising a cluster C91 sequence is capable of binding to beta-catenin in the presence of MDM2.

In some embodiments, the present disclosure provides a complex comprising beta-catenin, MDM2 and an agent of cluster C91. In some embodiments, the present disclosure provides methods for reducing levels of beta-catenin in a system, comprising administering or delivering to the system an effective amount of an agent of cluster C91. In some embodiments, the present disclosure provides a method for preventing or treating a condition, disorder or disease associated with beta-catenin, comprising administering or delivering to a subject suffering therefrom an effective amount of an agent of cluster C91.

### Cluster C92

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C92 as described herein. In some embodiments, an agent comprises X⁹²X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁹², X⁻¹, X⁰, X¹, X², X³, X⁴, X⁵, X⁶, X⁷, X⁸, X⁹, X¹⁰, X¹¹, X¹², X¹³, X¹⁴, X⁵, X¹⁶, and X¹⁷ is independently an amino acid residue.

In some embodiments, the present disclosure provides an agent comprising:

X⁹²X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁹², X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X³ comprises a side chain comprising an acidic group;
X⁵ comprises a hydrophobic side chain or a side chain comprising an aromatic group;
X⁹ comprises a hydrophobic side chain or a side chain comprising an aromatic group;
X¹² comprises a hydrophobic side chain; and
X¹⁵ comprises a hydrophobic side chain.

Various amino acid residues may be utilized for X⁹². For example, in some embodiments, X⁹² comprises a side chain comprising an acidic group. In some embodiments, a cluster is enriched for amino acid residues whose side chains comprise an acidic group at X⁹². In some embodiments, X⁹² comprises a side chain comprising —COOH. In some embodiments, X⁹² is D. In some embodiments, X⁹² is E.

Various amino acid residues may be utilized for X-1. For example, in some embodiments, X-1 comprises a side chain comprising a polar group. In some embodiments, a cluster is enriched for amino acid residues whose side chains comprise a polar group at X⁻¹. In some embodiments, X⁻¹ comprises a side chain comprising —OH. In some embodiments, X⁻¹ is S. In some embodiments, X⁻¹ comprises a side chain comprising —C(O)NH₂. In some embodiments, X⁻¹ is Q.

Various amino acid residues may be utilized for X⁰. For example, in some embodiments, the side chain of X⁰ is hydrophobic. In some embodiments, a cluster is enriched for amino acid residues comprising a hydrophobic side chain at X⁰. In some embodiments, the side chain of X⁰ is aliphatic. In some embodiments, the side chain of X⁰ is C₁₋₆ alkyl. In some embodiments, X⁰ is A. In some embodiments, X⁰ comprises a side chain comprising an aromatic group. In some embodiments, X⁰ is H. In some embodiments, X⁰ is F. In some embodiments, X⁰ is Y. In some embodiments, X⁰ is W.

Various amino acid residues may be utilized for X¹. For example, in some embodiments, X¹ comprises a side chain comprising a basic group. In some embodiments, a cluster is enriched for amino acid residues whose side chains comprise a basic group at X¹. In some embodiments, X¹ is R. In some embodiments, X¹ is H. In some embodiments, X¹ is K.

Various amino acid residues may be utilized for X³. In some embodiments, X³ comprises a side chain comprising —COOH. In some embodiments, X³ is D.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

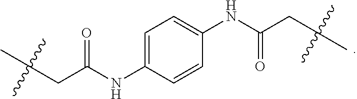 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ comprises a side chain comprising an aromatic group. In some embodiments, X⁵ is F. In some embodiments, X⁵ is Y.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising an aromatic group. In some embodiments, X⁹ is W. In some embodiments, X⁹ is F.

Various amino acid residues may be utilized for X¹⁰. For example, in some embodiments, X¹⁰ comprises a side chain comprising an acidic group. In some embodiments, a cluster is enriched for amino acid residues whose side chains comprise an acidic group at X¹⁰. In some embodiments, X¹⁰ comprises a side chain comprising —COOH. In some embodiments, X¹⁰ is D. In some embodiments, X¹⁰ is E. In some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —OH. In some embodiments, X¹⁰ is S. In some embodiments, X¹⁰ is T. In some embodiments, X¹⁰ comprises a side chain comprising —C(O)NH₂. In some embodiments, X¹⁰ is N. In some embodiments, X¹⁰ is Q.

Various amino acid residues may be utilized for X¹². In some embodiments, the side chain of X¹² is aliphatic. In some embodiments, the side chain of X¹² is C₁₋₆ alkyl. In some embodiments, X¹² is I. In some embodiments, X¹² is L.

Various amino acid residues may be utilized for X¹⁴. For example, in some embodiments, the side chain of X¹⁴ is hydrophobic. In some embodiments, a cluster is enriched for amino acid residues comprising a hydrophobic side chain at X¹⁴. In some embodiments, X¹⁴ comprises a side chain comprising an aromatic group. In some embodiments, X¹⁴ is F. In some embodiments, X¹⁴ is Y. In some embodiments, X¹⁴ is W.

Various amino acid residues may be utilized for X¹⁵. In some embodiments, X¹⁵ is M. In some embodiments, the side chain of X¹⁵ is aliphatic. In some embodiments, the side chain of X¹⁵ is C₁₋₆ alkyl. In some embodiments, X¹⁵ is I. In some embodiments, X¹⁵ is L.

Various amino acid residues may be utilized for X¹⁶. In some embodiments, X¹⁶ comprises a hydrophobic side chain or a side chain comprising an aromatic group. In some embodiments, X¹⁶ comprises a side chain comprising an aromatic group. In some embodiments, X¹⁶ is H. In some embodiments, X¹⁶ is Y. In some embodiments, X¹⁶ is W.

Various amino acid residues may be utilized for X¹⁷. For example, in some embodiments, X¹⁷ comprises a side chain comprising an acidic group. In some embodiments, a cluster is enriched for amino acid residues whose side chains comprise an acidic group at X¹⁷. In some embodiments, X¹⁷ comprises a side chain comprising —COOH. In some embodiments, X¹⁷ is D. In some embodiments, X¹⁷ comprises a side chain comprising a polar group. In some embodiments, a cluster is enriched for amino acid residues whose side chains comprise a polar group at X¹⁷. In some embodiments, X¹⁷ comprises a side chain comprising —OH. In some embodiments, X¹⁷ is T. In some embodiments, X¹⁷ comprises a side chain comprising —C(O)NH₂. In some embodiments, X¹⁷ is N. In some embodiments, X¹⁷ is Q.

In some embodiments, an agent described herein, e.g., a stapled peptide, interacts with one or more (e.g., 1, 2, 3, 4, 5, 6, 7, 8, 9, 10, 11, 12, 13, 14, 15, 16, or 17) or all of the following amino acid residues of MDM2 or amino acid residues corresponding thereto: Met50, Lys51, Leu54, Phe55, Leu57, Gly58, Ile61, Met62, Tyr67, Gln72, Val75, Val93, Lys94, His96, Ile99, Tyr100, and Tyr104. In some embodiments, it interacts with Met50 of MDM2 or an amino acid residue corresponding thereto. In some embodiments, it interacts with Lys51 of MDM2 or an amino acid residue corresponding thereto. In some embodiments, it interacts with Leu54 of MDM2 or an amino acid residue corresponding thereto. In some embodiments, it interacts with Phe55 of MDM2 or an amino acid residue corresponding thereto. In some embodiments, it interacts with Leu57 of MDM2 or an amino acid residue corresponding thereto. In some embodiments, it interacts with Gly58 of MDM2 or an amino acid residue corresponding thereto. In some embodiments, it interacts with Ile61 of MDM2 or an amino acid residue corresponding thereto. In some embodiments, it interacts with Met62 of MDM2 or an amino acid residue corresponding thereto. In some embodiments, it interacts with Tyr67 of MDM2 or an amino acid residue corresponding thereto. In some embodiments, it interacts with Gln72 of MDM2 or an amino acid residue corresponding thereto. In some embodiments, it interacts with Val75 of MDM2 or an amino acid residue corresponding thereto. In some embodiments, it interacts with Val93 of MDM2 or an amino acid residue corresponding thereto. In some embodiments, it interacts with Lys94 of MDM2 or an amino acid residue corresponding thereto. In some embodiments, it interacts with His96 of MDM2 or an amino acid residue corresponding thereto. In some embodiments, it interacts with Ile99 of MDM2 or an amino acid residue corresponding thereto. In some embodiments, it interacts with Tyr100 of MDM2 or an amino acid residue corresponding thereto. In some embodiments, it interacts with Tyr104 of MDM2 or an amino acid residue corresponding thereto. In some embodiments, an agent described herein, e.g., a stapled peptide, interacts with one or more (e.g., 1, 2, 3, 4, 5, or 6) or all of the following amino acid residues of beta-catenin or amino acid residues corresponding thereto: Glu620, Thr653, Tyr654, Ala656, Ala657, and Phe660. In some embodiments, it interacts with Glu620 of beta-catenin or an amino acid residue corresponding thereto. In some embodiments, it interacts with Thr653 of beta-catenin or an amino acid residue corresponding thereto. In some embodiments, it interacts with Tyr654 of beta-catenin or an amino acid residue corresponding thereto. In some embodiments, it interacts with Ala656 of beta-catenin or an amino acid residue corresponding thereto. In some embodiments, it interacts with Ala657 of beta-catenin or an amino acid residue corresponding thereto. In some embodiments, it interacts with Phe660 of beta-catenin or an amino acid residue corresponding thereto. In some embodiments, in a complex comprising an agent, beta-catenin and MDM2, one or more of Arg582, Val584, Arg587, Cys619, Gln623, Ala656, Ala657, Phe660, Glu664 and Asp665 of beta-catenin or amino acid residues corresponding thereto interact with one or more of Gln71, His73, Val93, Lys94, Glu95, His96 and Arg97 of MDM2 or amino acid residues corresponding thereto.

In some embodiments, an agent described herein, e.g., a stapled peptide, interacts with one or more (e.g., 1, 2, 3, 4, 5, 6, 7, 8, 9, 10, 11, or 12) or all of the following amino acid residues of MDM2 or amino acid residues corresponding thereto: Met50, Lys51, Leu54, Leu57, Gly58, Ile61, Gln72, Val75, Val93, Lys94, His96, and Tyr100. In some embodiments, it interacts with Met50 of MDM2 or an amino acid residue corresponding thereto. In some embodiments, it interacts with Lys51 of MDM2 or an amino acid residue corresponding thereto. In some embodiments, it interacts with Leu54 of MDM2 or an amino acid residue corresponding thereto. In some embodiments, it interacts with Leu57 of MDM2 or an amino acid residue corresponding thereto. In some embodiments, it interacts with Gly58 of MDM2 or an amino acid residue corresponding thereto. In some embodiments, it interacts with Ile61 of MDM2 or an amino acid residue corresponding thereto. In some embodiments, it interacts with Gln72 of MDM2 or an amino acid residue corresponding thereto. In some embodiments, it interacts with Val75 of MDM2 or an amino acid residue corresponding thereto. In some embodiments, it interacts with Val93 of MDM2 or an amino acid residue corresponding thereto. In some embodiments, it interacts with Lys94 of MDM2 or an amino acid residue corresponding thereto. In some embodiments, it interacts with His96 of MDM2 or an amino acid residue corresponding thereto. In some embodiments, it interacts with Tyr100 of MDM2 or an amino acid residue corresponding thereto. In some embodiments, an agent described herein, e.g., a stapled peptide, interacts with one or more (e.g., 1, 2, 3, 4, 5, 6, or 7) or all of the following amino acid residues of beta-catenin or amino acid residues corresponding thereto: Arg582, Glu620, Thr653, Tyr654, Ala656, Ala657, and Phe660. In some embodiments, it interacts with Arg582 of beta-catenin or an amino acid residue corresponding thereto. In some embodiments, it interacts with Glu620 of beta-catenin or an amino acid residue corresponding thereto. In some embodiments, it interacts with Thr653 of beta-catenin or an amino acid residue corresponding thereto. In some embodiments, it interacts with Tyr654 of beta-catenin or an amino acid residue corresponding thereto. In some embodiments, it interacts with Ala656 of beta-catenin or an amino acid residue corresponding thereto. In some embodiments, it interacts with Ala657 of beta-catenin or an amino acid residue corresponding thereto. In some embodiments, it interacts with Phe660 of beta-catenin or an amino acid residue corresponding thereto. In some embodiments, in a complex comprising an agent, beta-catenin and MDM2, one or more of Arg582, Val584, Arg587, Cys619, Gln623, Ala656, Ala657, Val658, Phe660 and Arg661 of beta-catenin or amino acid residues corresponding thereto interact with one or more of His73, Val93, Lys94, Glu95, His96 and Arg97 of MDM2 or amino acid residues corresponding thereto.

In some embodiments, an agent comprising a cluster C92 sequence is capable of binding to MDM2. In some embodiments, an agent comprising a cluster C92 sequence is capable of promoting an interaction between MDM2 and beta-catenin. In some embodiments, an agent comprising a cluster C92 sequence is capable of binding to beta-catenin in the presence of MDM2.

In some embodiments, the present disclosure provides a complex comprising beta-catenin, MDM2 and an agent of cluster C92. In some embodiments, the present disclosure provides methods for reducing levels of beta-catenin in a system, comprising administering or delivering to the system an effective amount of an agent of cluster C92. In some embodiments, the present disclosure provides a method for preventing or treating a condition, disorder or disease associated with beta-catenin, comprising administering or delivering to a subject suffering therefrom an effective amount of an agent of cluster C92.

### Cluster C93

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C93 as described herein. In some embodiments, an agent comprises X⁹³X⁻¹X⁰X¹X²X³X⁴X⁵X⁶X⁷X⁸X⁹X¹⁰X¹¹X¹²X¹³X¹⁴X¹⁵X¹⁶X¹⁷, wherein each of X⁹³, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent comprising:

X⁹³X⁻¹X⁰X¹X²X³X⁴X⁵X⁶X⁷X⁸X⁹X¹⁰X¹¹X¹²X¹³X¹⁴X¹⁵X¹⁶X¹⁷,

wherein:
each of X⁹³, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁵ comprises a hydrophobic side chain or a side chain comprising an aromatic group;
X⁹ comprises a hydrophobic side chain or a side chain comprising an aromatic group;
X¹¹ comprises a hydrophobic side chain;
X¹⁶ comprises a hydrophobic side chain or a side chain comprising an aromatic group; and
X¹⁷ comprises an acidic side chain.

Various amino acid residues may be utilized for X⁻¹. In some embodiments, X⁻¹ comprises a side chain comprising a polar group. In some embodiments, a cluster is enriched for amino acid residues whose side chains comprise a polar group at X⁻¹. In some embodiments, X⁻¹ comprises a side chain comprising —OH. In some embodiments, X⁻¹ is T. In some embodiments, the side chain of X⁻¹ is hydrophobic. In some embodiments, a cluster is enriched for amino acid residues comprising a hydrophobic side chain at X⁻¹. In some embodiments, the side chain of X⁻¹ is aliphatic. In some embodiments, the side chain of X⁻¹ is C₁₋₆ alkyl. In some embodiments, X⁻¹ is I. In some embodiments, X⁻¹ is L. In some embodiments, X⁻¹ comprises a side chain comprising an aromatic group. In some embodiments, X⁻¹ is W.

Various amino acid residues may be utilized for X⁰. For example, in some embodiments, X⁰ comprises a side chain comprising a polar group. In some embodiments, a cluster is enriched for amino acid residues whose side chains comprise a polar group at X⁰. In some embodiments, X⁰ comprises a side chain comprising —OH. In some embodiments, X⁰ is S. In some embodiments, X⁰ is T. In some embodiments, X⁰ comprises a side chain comprising —C(O)NH₂. In some embodiments, X⁰ is N. In some embodiments, X⁰ is Q.

Various amino acid residues may be utilized for X¹. For example, in some embodiments, X¹ comprises a side chain comprising an acidic group. In some embodiments, a cluster is enriched for amino acid residues whose side chains comprise an acidic group at X¹. In some embodiments, X¹ comprises a side chain comprising —COOH. In some embodiments, X¹ is D. In some embodiments, X¹ comprises a side chain comprising a polar group. In some embodiments, a cluster is enriched for amino acid residues whose side chains comprise a polar group at X¹. In some embodiments, X¹ comprises a side chain comprising —C(O)NH₂. In some embodiments, X¹ is Q.

Various amino acid residues may be utilized for X². For example, in some embodiments, X² comprises a side chain comprising a polar group. In some embodiments, a cluster is enriched for amino acid residues whose side chains comprise a polar group at X². In some embodiments, X² comprises a side chain comprising —OH. In some embodiments, X² is S. In some embodiments, X² is T. In some embodiments, X² comprises a side chain comprising —C(O)NH₂. In some embodiments, X² is N. In some embodiments, X² is Q.

Various amino acid residues may be utilized for X³. For example, in some embodiments, the side chain of X³ is hydrophobic. In some embodiments, a cluster is enriched for amino acid residues comprising a hydrophobic side chain at X³. In some embodiments, the side chain of X³ is aliphatic. In some embodiments, the side chain of X³ is C₁₋₆ alkyl. In some embodiments, X³ is A. In some embodiments, X³ is V. In some embodiments, X³ is I. In some embodiments, X³ comprises a side chain comprising an aromatic group. In some embodiments, X³ is Y. In some embodiments, X³ is W.

In some embodiments, X⁴ is a residue for stapling. In some embodiments, X⁴ is stapled with an amino acid residue. In some embodiments, X¹¹ is a residue for stapling. In some embodiments, X¹¹ is stapled with an amino acid residue. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

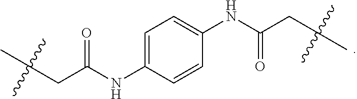 [SMILES: *CC(=O)Nc1ccc(NC(=O)C*)cc1]

In some embodiments, X⁴ and X¹¹ are stapled through a non-cysteine staple.

Various amino acid residues may be utilized for X⁵. In some embodiments, X⁵ comprises a side chain comprising an aromatic group. In some embodiments, X⁵ is F. In some embodiments, X⁵ is W.

Various amino acid residues may be utilized for X⁶. For example, in some embodiments, X⁶ comprises a side chain comprising a polar group. In some embodiments, a cluster is enriched for amino acid residues whose side chains comprise a polar group at X⁶. In some embodiments, X⁶ comprises a side chain comprising —C(O)NH₂. In some embodiments, X⁶ is Q. In some embodiments, X⁶ is P.

Various amino acid residues may be utilized for X⁷. In some embodiments, X⁷ is A.

Various amino acid residues may be utilized for X⁸. In some embodiments, X⁸ is A.

Various amino acid residues may be utilized for X⁹. In some embodiments, X⁹ comprises a side chain comprising an aromatic group. In some embodiments, X⁹ is W.

Various amino acid residues may be utilized for X¹⁰. For example, in some embodiments, X¹⁰ comprises a side chain comprising a polar group. In some embodiments, a cluster is enriched for amino acid residues whose side chains comprise a polar group at X¹⁰. In some embodiments, X¹⁰ comprises a side chain comprising —C(O)NH₂. In some embodiments, X¹⁰ is Q. In some embodiments, X¹⁰ is M.

Various amino acid residues may be utilized for X¹². In some embodiments, X¹² comprises a hydrophobic side chain. In some embodiments, the side chain of X¹² is aliphatic. In some embodiments, the side chain of X¹² is C₁₋₆ alkyl. In some embodiments, X¹² is V. In some embodiments, X¹² is I. In some embodiments, X¹² is L.

Various amino acid residues may be utilized for X¹³. For example, in some embodiments, X¹³ comprises a side chain comprising a polar group. In some embodiments, a cluster is enriched for amino acid residues whose side chains comprise a polar group at X¹³. In some embodiments, X¹³ comprises a side chain comprising —OH. In some embodiments, X¹³ is S. In some embodiments, X¹³ is T. In some embodiments, X¹³ comprises a side chain comprising —C(O)NH₂. In some embodiments, X¹³ is Q.

Various amino acid residues may be utilized for X¹⁴. For example, in some embodiments, X¹⁴ comprises a side chain comprising an acidic group. In some embodiments, a cluster is enriched for amino acid residues whose side chains comprise an acidic group at X¹⁴. In some embodiments, X¹⁴ comprises a side chain comprising —COOH. In some embodiments, X¹⁴ is D.

Various amino acid residues may be utilized for X¹⁵. For example, in some embodiments, X¹⁵ comprises a side chain comprising a polar group. In some embodiments, a cluster is enriched for amino acid residues whose side chains comprise a polar group at X¹⁵. In some embodiments, X¹⁵ comprises a side chain comprising —OH. In some embodiments, X¹⁵ is T.

Various amino acid residues may be utilized for X¹⁶. In some embodiments, X¹⁶ comprises a side chain comprising an aromatic group. In some embodiments, X¹⁶ is F. In some embodiments, X¹⁶ is Y. In some embodiments, X¹⁶ is W.

Various amino acid residues may be utilized for X¹⁷. In some embodiments, X¹⁷ comprises a side chain comprising —COOH. In some embodiments, X¹⁷ is D. In some embodiments, X¹⁷ is E.

In some embodiments, an agent comprising a cluster C93 sequence is capable of binding to MDM2. In some embodiments, an agent comprising a cluster C93 sequence is capable of promoting an interaction between MDM2 and beta-catenin. In some embodiments, an agent comprising a cluster C93 sequence is capable of binding to beta-catenin in the presence of MDM2.

In some embodiments, the present disclosure provides a complex comprising beta-catenin, MDM2 and an agent of cluster C93. In some embodiments, the present disclosure provides methods for reducing levels of beta-catenin in a system, comprising administering or delivering to the system an effective amount of an agent of cluster C93. In some embodiments, the present disclosure provides a method for preventing or treating a condition, disorder or disease associated with beta-catenin, comprising administering or delivering to a subject suffering therefrom an effective amount of an agent of cluster C93.

Among other things, provided technologies can provide various benefits and advantages, e.g., selective binding. In some embodiments, a trimerizer agent selectively forms a complex with two polypeptides compared to each of the polypeptide alone. In some embodiments, a trimerizer agent binds to an individual polypeptide weakly or do not bind. In some embodiments, a trimerizer agent selectively forms complex with a pair of polypeptide compared to another pair. In some embodiments, the present disclosure provides technologies that bind VHL-ELOBC. In some embodiments, the present disclosure provides technologies that bind VHL-ELOBC selectively over SOCS2-ELOBC. In some embodiments, provided technologies do not compete with HXC78.

### Griptides

In some embodiments, the present disclosure provides technologies for identification, characterization, production, and/or use of griptide agents. In some embodiments, a griptide comprises two paired alpha-helices. In some embodiments, a griptide comprises two stapled peptides portions, each forming an alpha-helix structure. In some embodiments, a griptide provides a larger surface compared to agent comprising a single alpha-helix structure. In some embodiments, a griptide can engage a target with high affinity and/or specificity. In some embodiments, a griptide can engage a challenging target with high affinity and specificity, e.g., compared to an agent comprising a single alpha-helix structure.

In some embodiments, the present disclosure provides a method comprising:
contacting a collection of stapled peptides with a target of interest so that a number of stapled peptides of the collection binds to the target; and
determining amino acid sequences of stapled peptides that bind to the target.

In some embodiments, one or more stapled peptides, e.g., about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of all stapled peptides, independently comprise two portions, wherein the peptide backbones of the two portions do not overlap, and each portion independently is or comprises an alpha-helical structure. In some embodiments, one or more stapled peptides, e.g., about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of all stapled peptides, independently comprise two portions, wherein the peptide backbones of the two portions do not overlap, and each portion independently comprises two residues stapled or suitable for stapling. In some embodiments, one or more stapled peptides, e.g., about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of all stapled peptides, independently comprise two portions, wherein the peptide backbones of the two portions do not overlap, and each portion independently comprises two stapled residues. In some embodiments, each portion independently forms or comprises or is part of an alpha-helical structure. In some embodiments, each portion is independently a moiety of a stapled peptide as described herein. In some embodiments, each portion independently comprises an amino acid sequence and a staple as described herein. For example, in some embodiments, each portion is independently about 5-30, about 5-20, about 8-20, or about 5, 6, 7, 8, 9, 10, 11, 12, 13, 14, 15, 16, 17, 18, 19, 20, 21, 22, 23, 24, 25, 26, 27, 28, 29 or 30 amino acid residues in length. In some embodiments, each portion is independently about 8-20 amino acid residues in length.

In some embodiments, each of the two portions independently comprises an amino acid residue suitable for crosslinking. In some embodiments, the two portions are crosslinked. In some embodiments, an amino acid residue suitable for crosslinking is cysteine. In some embodiments, two portions are crosslinked through formation of a disulfide bond between two cysteine residues.

In some embodiments, the two portions are of two different peptide chains. In some embodiments, the two portions are ports of the same peptide chains. In some embodiments, peptide backbones of the two portions are two portions of the same peptide backbone. In some embodiments, the two portions are tandem. In some embodiments, each of them is independently part of two peptide backbones.

In some embodiments, the present disclosure provides a method comprising:
contacting a collection of stapled peptides with a target of interest so that a number of stapled peptides of the collection binds to the target; and
determining amino acid sequences of stapled peptides that bind to the target;
wherein one or more stapled peptides independently comprise an amino acid residue suitable for crosslinking with other stapled peptides, or one or more stapled peptides are independently crosslinked with one or more other stapled peptides.

In some embodiments, an amino acid residue suitable for crosslinking is cysteine. In some embodiments, one or more stapled peptides are independently crosslinked with one or more other stapled peptides. In some embodiments, crosslinking is through cysteine residues. In some embodiments, about or at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of all stapled peptides in the collection are crosslinked.

In some embodiments, a peptide is a dimer. In some embodiments, a peptide is a homodimer. In some embodiments, a peptide is a heterodimer.

### Treatment of Conditions, Disorders or Diseases

In some embodiments, certain stapled peptides and/or other agents as described herein are useful for preventing or treating various conditions, disorders or diseases. In some embodiments, a therapeutic composition as described herein, that comprises and/or delivers a stapled peptide (e.g., a non-cysteine stapled peptide, for example corresponding to a parent cysteine stapled peptide identified and/or characterized as described herein) is administered to a subject in need thereof. In some embodiments, a subject may have received and/or be receiving other therapy so that, in some embodiments, therapy with a provided peptide agent may be administered in combination with other therapy; in some such embodiments, a peptide agent as described herein may be administered via a composition that contains and/or delivers another therapeutic agent; in some embodiments, separate compositions are employed.

Various strategies for administering stapled peptides are available in the art and can be utilized in accordance with the present disclosure. In some embodiments, provided peptides have enhanced cell penetration properties compared otherwise identical but non-stapled peptides.

In some particular embodiments, certain provided stapled peptides and/or other technologies are useful to bind to and/or modulate one or more activities or effects of, beta-catenin.

In some embodiments, certain provided stapled peptides and/or other technologies are useful in treatment of a condition, disorder, or disease associated with one or more components involved in Wnt/beta-catenin signaling and/or specifically with one or more beta-catenin functions.

In some embodiments, certain provided stapled peptides and/or other technologies are useful in modulating, e.g., reducing the interaction between beta-catenin and another protein, for example, Axin, APC, BCL9, TCF4/TCF7L2, TCF3/TCF7L1, and TCF7.

In some embodiments, a condition, disorder or disease is associated with beta-catenin. In some embodiments, a condition, disorder or disease is associated with RNF31. In some embodiments, a condition, disorder or disease is associated with CDK2. In some embodiments, a condition, disorder or disease is associated with PPIA. In some embodiments, a condition, disorder or disease is associated with PD-L1. In some embodiments, a condition, disorder or disease is associated with an E3 ligase. Various conditions, disorders or diseases associated with such polypeptides have been reported and can be prevented or treated in accordance with the present disclosure.

In some embodiments, a condition, disorder or disease is selected from cancer, cardiac disease, dilated cardiomyopathy, fetal alcohol syndrome, depression, and diabetes. In some embodiments, a condition, disorder, or disease is a heart condition, disorder, or disease. In some embodiments, a condition, disorder, or disease is cancer. In some embodiments a cancer is selected from: colon cancer, colorectal cancer, rectal cancer, prostate cancer familial adenomatous polyposis (FAP), Wilms Tumor, melanoma, hepatocellular carcinoma, ovarian cancer, endometrial cancer, medulloblastoma pilomatricomas, primary hetpatocellular carcinoma, ovarial carcinoma, breast cancer, lung cancer, glioblastoma, pliomatrixoma, medulloblastoma, thyroid tumors, ovarian neoplasms. In some embodiments, a cancer is colorectal cancer. In some embodiments, a cancer is hepatocellular cancer. In some embodiments, a cancer is prostate cancer. In some embodiments, a cancer is melanoma. In some embodiments, a cancer is associated with Wnt/beta-catenin signaling. In some embodiments, a cancer is associated with beta-catenin. As appreciated by those skilled in the art, provided technologies are applicable to various targets, including many protein targets associated with cancer.

### EXEMPLIFICATION

Those skilled in the art appreciate that various technologies are available for designing, identifying, characterizing, manufacturing, assessing, using, etc., provided agents, collections, methods, etc. in accordance with the present disclosure. Described below are certain such useful technologies.

### Example 1. Technologies for Preparing, Assessing and Identifying Stapled Peptides that can Bind Various Targets and Collections Thereof

Recent advances in identifying human disease targets have not been matched by advances in the ability to drug these targets. This actionability gap is largely due to the fact that neither of the two main classes of approved therapeutics—biologics and small molecules—can simultaneously address target accessibility and selective target engagement. Biologics, despite an impressive ability to engage diverse target proteins, are largely restricted to an extracellular operating theatre, as their size and polarity renders them unable to cross biological membranes. Small molecules, in contrast, can access the intracellular space, but cannot bind with high affinity and specificity to the vast majority of proteins that are found there.

Among other things, the present disclosure encompasses the disconnect between the ability to identify disease targets and the ability to drug them with high strength and specificity. In some embodiments, the present disclosure provides technologies that can provide new classes of drugs—ones that can engage intracellular proteins that lack the deep hydrophobic pocket ordinarily required for small-molecule binding.

In nature, such “undruggable” proteins are often targeted with macrocyclic molecules, frequently peptidic in structure, whose large size compared to small molecules enables them to bind with high affinity and specificity to protein surfaces. Significant efforts have been made to elucidate the mechanisms of cell entry for these natural products, which typically possess molecular weights of 700-1,200 Da or higher, well beyond the typical range for cell penetration in small-molecule drug discovery. While the mechanisms of cell entry are complex and vary from molecule to molecule, certain research on peptidic macrocycles has been reported to highlight the importance of desolvating amide protons and reducing their exposure to the membrane interior as a key driver in passive, thermal diffusion across the lipid bilayer—a phenomenon that may be referred to as amide-proton cloaking. In some embodiments, the amide proton, present between residues in a polypeptide chain, is highly electropositive and forms a strong hydrogen-bonding interaction with water. In some embodiments, this poses a substantial hurdle for membrane permeability, since tightly bound solvent water molecules are shed prior to entering the lipid bilayer. In some embodiments, exposed amide groups incur a further energetic penalty upon membrane entry due to unfavorable electrostatic interactions with the low-dielectric environment of the membrane interior. Consequently, many peptides and proteins are unable to cross membranes.

In some embodiments, the present disclosure provides agents, e.g., stapled peptides, that can cross membranes. In some embodiments, provided agents comprises staples. In some embodiments, staples facilitate formations and/or maintenance, or stabilize alpha-helical structures.

Among other things, the present disclosure encompasses the recognition that design of stapled peptides can be challenging in view of the inability to systematically define the alpha-helix binding sites on a protein's surface, and to identify stapled peptides that bind to those sites. In some embodiments, this limitation has restricted development of stapled peptides and their uses by many third parties to only those protein targets for which naturally occurring or previously characterized alpha-helical binders were known, with stapled peptides generated from fragments of the known binders. Among other things, the present disclosure provide a rapid, high-throughput platform, in some embodiments, utilizing phage display, that enables an unbiased mapping of interactome of a target of interest, e.g., a protein, with agents such as stapled peptides, without requiring prior knowledge of the structure or known binding partners of a target of interest. Among other things, provided technologies are capable of identifying alpha-helix-binding sites on the surfaces of a range of protein folds, including many for which no alpha-helical binders are reported. In some embodiments, stapled peptides that bind these sites are able to impact diverse functions, including inhibiting interactions (e.g., protein-protein interactions), inhibiting enzymatic activity, inducing dimerization, and inducing conformational changes. In some embodiments, a range of binding modes can be adopted. In some embodiments, a mode is “side-on”, i.e., mediated exclusively by stapled peptide side-chains rather than involving main chain amide interactions. Among other things, provided technologies significantly expands the universe of targets of interest including proteins that can be bound by stapled peptides, and furthers the pursuit of targeting undruggable targets of interest including proteins.

Among other things, the present disclosure provides technologies for preparing, assessing, characterizing and identifying stapled peptides that can bind various targets. In some embodiments, provided stapled peptides comprise alpha-helical structures. In some embodiments, stapled peptides may be referred to as “Helicons.” In some embodiments, the present disclosure provides unbiased screening platform for identifying stapled peptide binders. In some embodiments, the present disclosure provides libraries comprising a number of (e.g., about or at least about 100, 500, 1000, 5000, 10⁴, 10⁵, 10⁶, 10⁷, 10⁸, 10⁹, 10¹⁰, 10¹¹, 10¹², etc.) stapled peptides. Various technologies can be utilized to screen provided libraries. In one example, a phage display technology was utilized for preparing and/or screening libraries comprising stapled peptides. In some embodiments, a phage display technology can provide i) high library diversities, ii) tolerance to chemical manipulation, and/or iii) rapid and inexpensive library generation. In some embodiments, the present disclosure utilizes technologies that are compatible with primary amines and other reactive groups present on phage particles. In some embodiments, a stapling system based on crosslinking cysteine residues with electrophiles, e.g., alkyl bromides, was utilized. In some embodiments, such chemistry can be performed under aqueous conditions and in the presence of other biomacromolecules. Various bifunctional electrophiles are available and may be utilized in accordance with the present disclosure, e.g., those described in WO 2020/041270, the stapling technologies, e.g., electrophiles, of which are incorporated herein by reference. A panel of bifunctional alkyl bromides was utilized for stapling. In one example, N,N′-(1,4-phenylene)bis(2-bromoacetamide) was utilized for crosslinking pairs of cysteines at i,i+7 positioning. In some embodiments, incorporating this staple into a diverse panel of peptides, e.g., those are 14 amino acid residues in length, consistently led to an increase in alpha-helical secondary structure, as assessed by circular dichroism.

In some embodiments, this chemistry was performed on phage-displayed Helicons. M13 bacteriophage was prepared with a model sequence containing a pair i,i+7 cysteine residues fused to a pIII coat protein. In some embodiments, one or more phage-displayed polypeptides with pairs of cysteines were independently present in the oxidized, disulfide-bonded state. In some embodiments, timing and concentration of phage was optimized. In some embodiments, choice of reducing agent was optimized. In some embodiments, choice of crosslinker was optimized. In one example, on-phage crosslinking was achieved by briefly treating the phage with dithiothreitol (DTT), followed by rapid dialysis, incubation with a bifunctional electrophile, e.g., a bifunctional alkyl bromide such as N,N′-(1,4-phenylene)bis(2-bromoacetamide), and a final capping and quenching step. Stapling of desired cysteine residues was achieved despite the presence of multiple structural disulfide bonds in the pIII protein. In some embodiments, presence of properly crosslinked Helicons was confirmed by trypsinization of these phage followed by mass spectrometry (MS) analysis. In some embodiments, phage prepared in this way remained viable and compatible with downstream screening and sequencing. In some embodiments, stapled peptides interact with their targets with alpha-helical modes. In some embodiments, stapled peptides interact with their targets with non-alpha-helical modes. In some embodiments, it was observed that in many co-crystal structures solved of stapled peptides discovered using phage libraries prepared with this crosslinker, a majority of stapled peptides bind their targets with mostly or entirely alpha-helical modes.

Agents, e.g., stapled peptides, can be characterized utilizing various technologies in accordance with the present disclosure. In some embodiments, stapled peptides and unstapled peptides were characterized by HPLC, mass spectrometry, etc. In some embodiments, crosslinked peptides displayed on phage were characterized by mass spectrometry, confirming on-phage cysteine stapling. In some embodiments, sequencing of 12 phage library members indicates that 11 of 12 sequences match the expected library design (with one sequence containing an additional amino acid insertion).

As demonstrated herein, provided technologies allow for swift, unbiased screening of large Helicon libraries, e.g., using phage display. Among other things, provided technologies are able to identify previously unknown alpha-helix binding sites on a range of targets of interest including protein folds and types, including a transcriptional regulator (beta-catenin), two structurally dissimilar domains from an E3 ubiquitin ligase (RNF31), a kinase (CDK2), a peptidyl-prolyl cis-trans isomerase (PPIA), and an extracellular receptor (PD-L1). X-ray co-crystal structures indicate that many of these sites are located on surfaces not previously known to bind alpha-helices. Among other things, biochemical and structural experiments demonstrate diverse functional impacts by the Helicons which bind these sites, including inhibition of protein-protein interactions, inhibition of enzymatic activity, induced conformational rearrangement, and induced dimerization. Additional screens have been successfully performed with a wide range of small molecule, peptidic, and protein binding partners, and phosphorylated vs. non-phosphorylated, glycosylated vs. non-glycosylated, apo vs. ligand-bound, mouse vs. human, monomeric vs. multimeric proteins, etc. were also assessed. In some embodiments, one or more panning rounds prior to screen, e.g., the multiwell screen, can be introduced to enrich low-abundance library members and provide clearer signals to select binders with, and the use of monovalent phagemid systems can avoid the avidity effects of the multivalent phage system that can complicate the interpretation of apparent on-phage affinities.

Among other things, it was observed many Helicons were alpha-helical in the region of the molecule involved in engaging the target, with few examples of fraying at the ends of Helicons outside of the stapled region. In some embodiments, significant exposure of amide protons in a Helicon structure, except those at the N-terminus of each Helicon, is not observed. In some embodiments, Helicons engage target surface, e.g., protein surface, by side chains and not by any Helicon main-chain amides, e.g., as shown in crystal structures.

In some embodiments, the present disclosure provides technologies for establishment of structure-activity relationships to identify which features are critical for binding and activity. In the absence of a co-crystal structure or other direct structural information, establishing structure-activity relationships can requires extensive empirical testing of compound analogs. Due to the relatively large number of Helicons in many of the clusters discovered in the provided technologies, in some embodiments, sufficient information in clusters can be utilized to predict which amino acids are involved, e.g., in target binding. As demonstrated herein, comparison of clusters with co-crystal structures confirmed that in almost all cases, highly conserved amino acids were directly participating in protein binding.

Certain stapled peptides that were identified to bind their targets are described herein as examples. Certain agents including stapled peptides are presented below as examples (including some controls).

TABLE 1
Certain agents including stapled peptides.

| | | | SEQ | Expected | Observed | |
|---|---|---|---|---|---|---|
| Target | FOG ID | Sequence | ID NO: | Mass | Mass | Staple type |
| CTNNB1 | FP01567 | Ac-DPATHRCEWAALHCELV-NH2 | 46 | 2180.4 | 2180.2 | Cys- |
| | | | | | | stapled |
| CTNNB1 | FP49332 | Ac-DPATHRCEWAALHCELV-NH2 | 47 | 1992.2 | 1989.2 | unstapled |
| CTNNB1 | FP49250 | Ac-DPAAHRCEWAALHCELV-NH2 | 48 | 2150.4 | 2149.0 | Cys- |
| | | | | | | stapled |
| CTNNB1 | FP49251 | Ac-DPATARCEWAALHCELV-NH2 | 49 | 2114.4 | 2112.8 | Cys- |
| | | | | | | stapled |
| CTNNB1 | FP49252 | Ac-DPATHACEWAALHCELV-NH2 | 50 | 2095.3 | 2094.0 | Cys- |
| | | | | | | stapled |
| CTNNB1 | FP49253 | Ac-DPATHRCAWAALHCELV-NH2 | 51 | 2122.4 | 2120.8 | Cys- |
| | | | | | | stapled |
| CTNNB1 | FP49254 | Ac-DPATHRCEAAALHCELV-NH2 | 52 | 2065.3 | 2063.8 | Cys- |
| | | | | | | stapled |
| CTNNB1 | FP49255 | Ac-DPATHRCEWAAAHCELV-NH2 | 53 | 2138.3 | 2136.8 | Cys- |
| | | | | | | stapled |
| CTNNB1 | FP49257 | Ac-DPATHRCEWAALHCALV-NH2 | 54 | 2122.4 | 2120.8 | Cys- |
| | | | | | | stapled |
| CTNNB1 | FP49258 | Ac-DPATHRCEWAALHCEAV-NH2 | 55 | 2138.3 | 2136.8 | Cys- |
| | | | | | | stapled |
| CTNNB1 | FP49259 | Ac-DPATHRCEWAALHCELA-NH2 | 56 | 2152.4 | 2150.8 | Cys- |
| | | | | | | stapled |
| CTNNB1 | FP01822 | Ac-DPASELCEWAAIHCDLV-NH2 | 57 | 2101.3 | 2100.2 | Cys- |
| | | | | | | stapled |
| CTNNB1 | FP49333 | Ac-DPASELCEWAAIHCDLV-NH2 | 58 | 1913.1 | 1912.1 | unstapled |
| CTNNB1 | FP01838 | Ac-DPAILECHIAAWNCYEI-NH2 | 59 | 2190.5 | 2189.0 | Cys- |
| | | | | | | stapled |
| CTNNB1 | FP49194 | \|Ac-DPATLDCHIAAWDCWDE-NH2 | 60 | 2190.3 | 2188.8 | Cys- |
| | | | | | | stapled |
| CTNNB1 | FP49193 | Ac-DPAILACHLAAMDCSDW-NH2 | 61 | 2061.3 | 2060.4 | Cys- |
| | | | | | | stapled |
| CTNNB1 | FP49432 | Ac-DPAILACHLAAMDCSDW-NH2 | 62 | 1873.1 | 1871.2 | unstapled |
| CTNNB1 | FP49196 | Ac-DPANANCILAAHECRIW-NH2 | 63 | 2126.4 | 2124.8 | Cys- |
| | | | | | | stapled |
| CTNNB1 | FP49197 | Ac-DPAEVECMLAAHVCRAF-NH2 | 64 | 2091.4 | 2089.8 | Cys- |
| | | | | | | stapled |
| CTNNB1 | FP01848 | Ac-DPAQDDCILAAHVCALW-NH2 | 65 | 2070.3 | 2069.1 | Cys- |
| | | | | | | stapled |
| CTNNB1 | FP49433 | Ac-DPAQDDCILAAHVCALW-NH2 | 66 | 1882.1 | 1881.1 | unstapled |
| CTNNB1 | FP49199 | Ac-DPADWECEHAALLCHYW- | 67 | 2288.5 | 2286.9 | Cys- |
| | | NH2 | | | | stapled |
| CTNNB1 | FP49200 | Ac-DPALWQCEHAALLCDVH-NH2 | 68 | 2150.4 | 2149.2 | Cys- |
| | | | | | | stapled |
| CTNNB1 | FP49431 | Ac-DPALWQCEHAALLCDVH-NH2 | 69 | 1962.2 | 1959.5 | |
| CTNNB1 | FP05863 | Ac-DPAIIHCYEAAFFCQYI-NH2 | 70 | 2233.5 | 2233.4 | Cys- |
| | | | | | | stapled |
| CTNNB1 | FP05874 | Ac-DPAVMECYEAAFICHYV-NH2 | 71 | 2190.5 | 2189.3 | Cys- |
| | | | | | | stapled |
| CTNNB1 | FP04872 | 5FAM-bAla- | 72 | 5245.3 | 5242.2 | Cys- |
| | | DDLGANDELISFKDEGEQEEKSSE | | | | stapled |
| | | NSSAERDLADVKSSLVNESE-NH2 | | | | |
| CTNNB1 | FP00013 | FITC-PEG1-PQ-S5-ILD-S5- | 73 | 2358.8 | 2357.8 | S5-S5 |
| | (fStAx- | HVRRVWR | | | | stapled |
| | 33⁽¹⁰⁾) | | | | | |
| RNF31 | FP06635 | Ac-DPAEWICRMAAMNCLYQ-NH2 | 74 | 2244.6 | 2244.4 | Cys- |
| (PUB) | | | | | | stapled |
| RNF31 | FP49434 | Ac-DPAEWICRMAAMNCLYQ-NH2 | 75 | 2056.4 | 2054.2 | unstapled |
| (PUB) | | | | | | |
| RNF31 | FP06641 | Ac-DPARDWCLYAAYDCYTA- | 76 | 2226.4 | 2226.3 | Cys- |
| (PUB) | | NH2 | | | | stapled |
| RNF31 | FP49435 | Ac-DPARDWCLYAAYDCYTA- | 77 | 2038.2 | 2035.9 | unstapled |
| (PUB) | | NH2 | | | | |
| RNF31 | FP06649 | Ac-DPAFTDCQLAAAVCMTY-NH2 | 78 | 2049.3 | 2049.5 | Cys- |
| (PUB) | | | | | | stapled |
| RNF31 | FP06652 | Ac-DPAIVQCAWAALYCDMQ-NH2 | 79 | 2127.4 | 2127.3 | Cys- |
| (PUB) | | | | | | stapled |
| RNF31 | FP16923 | FITC-NHHex- | 80 | 2824.0 | 2822.0 | Cys- |
| (PUB) | | AEHEEDMYRAADEIEKEKE-NH2 | | | | stapled |
| RNF31 | FP06655 | Ac-DPAMQRCFSAAVYCAIS-NH2 | 81 | 2062.3 | 2062.3 | Cys- |
| (UBA) | | | | | | stapled |
| RNF31 | FP12122 | 5FAM-bAla- | 82 | 2449.7 | 2449.8 | Cys- |
| (UBA) | | DPAMQRCFSAAVYCAIS-NH2 | | | | stapled |
| CDK2 | FP24322 | Ac-FECLDAFFSC-NH2 | 83 | 1410.6 | 1409.8 | Cys- |
| | | | | | | stapled |
| CDK2 | FP19711 | Ac-DPAWWVCAIAAIECSDV-NH2 | 84 | 2078.3 | 2077.4 | Cys- |
| | | | | | | stapled |
| CDK2 | FP33215 | Ac-WWVCAIAAIECSD-NH2 | 85 | 1695.9 | 1695.0 | Cys- |
| | | | | | | stapled |
| PPIA | FP29103 | Ac-PDCHIRAYVCH-NH2 | 86 | 1542.7 | 1542.1 | Cys- |
| | | | | | | stapled |
| PPIA | FP29092 | Ac-DPANQDCHVAAWHCWQR- | 87 | 2266.4 | 2265.8 | Cys- |
| | | NH2 | | | | stapled |
| PPIA | FP29102 | Ac-PECHIEA YWCI-NH2 | 88 | 1592.8 | 1591.8 | Cys- |
| | | | | | | stapled |
| PD-L1 | FP30790 | Ac-DPAAADCQWAAFLCRVY-NH2 | 89 | 2129.4 | 2128.0 | Cys- |
| | | | | | | stapled |
| PD-L1 | FP28132 | Ac-DPALWQCVFAARSCYEE-NH2 | 90 | 2217.4 | 2217.0 | Cys- |
| | | | | | | stapled |
| PD-L1 | FP28141 | Ac-DPALAQCVFAARSCYEE-NH2 | 91 | 2102.3 | 2102.5 | Cys- |
| | | | | | | stapled |
| PD-L1 | FP28135 | Ac-DPALWMCVFAARQCYES-NH2 | 92 | 2219.5 | 2218.9 | Cys- |
| | | | | | | stapled |
| PD-L1 | FP28136 | Ac-DPALWQCVFAARYCYEE-NH2 | 93 | 2293.5 | 2293.2 | Cys- |
| | | | | | | stapled |
| PRKCI | FP47831 | 5FAM-QRFARKGALRQKNV-NH2 | 94 | 2028.0 | 2029.2 | unstapled |
| (5FAM- | | | | | | |
| PKC | | | | | | |
| iota/zeta- | | | | | | |
| blocking | | | | | | |
| peptide) | | | | | | |
| NA | FP47842 | 5FAM-dGlu-dGln-dLys-dLeu-dIle- | 95 | 1559.7 | 1561.1 | unstapled |
| (F5AM- | | dSer-dGlu-dGlu-dAsp-dLeu-NH2 | | | | |
| D-Myc | | | | | | |
| peptide) | | | | | | |
| NA | FP47843 | 5FAM-dTyr-dPro-dTyr-dAsp-dVal- | 96 | 1458.5 | 1459.8 | unstapled |
| (5FAM- | | dPro-dAsp-dTyr-dAla-NH2 | | | | |
| D-HA | | | | | | |
| peptide) | | | | | | |
| MDM2 | FP47839 | Ac-[5FAM]-Lys-Gln-Ser-Gln-Gln- | 97 | 2594.3 | 2596.4 | R8-S5⁽¹¹⁾ |
| (5FAM- | | Thr-Phe-R8-Asn-Leu-Trp-Arg-Leu- | | | | stapled |
| SAH-p53-8) | | Leu-S5-Gln-Asn-NH2 | | | | |
| MDM2 | FP01413 | Ac-Leu-Thr-Phe-R8-Glu-Tyr-Trp- | 98 | 1743.9 | 1745.0 | R8-S5 |
| (ATSP- | | Ala-Gln-Cba-S5-Ser-Ala-Ala-NH2 | | | | stapled |
| 7041) | | | | | | |
| CTNNB1 | FP07855 | Ac-Val-nLeu-Glu-R8-Tyr-Glu-Ala- | 99 | 1786.0 | 1785.7 | R8-S5 |
| | | Ala-Phe-Ile-S5-His-Tyr-Val-NH2 | | | | stapled |
| CTNNB1 | FP07856 | Ac-Val-nLeu-Glu-R8-Tyr-Glu-Ala- | 99 | 1786.0 | 1785.8 | R8-S5 |
| | | Ala-Phe-Ile-S5-His-Tyr-Val-NH2 | | | | stapled |
| CTNNB1 | FP07859 | Ac-Ile-Leu-Gln-R8-Tyr-Glu-Ala- | 100 | 1801.9 | 1801.6 | R8-S5 |
| | | Ala-Phe-Ser-S5-His-Tyr-Gln-NH2 | | | | stapled |
| CTNNB1 | FP07860 | Ac-Ile-Leu-Gln-R8-Tyr-Glu-Ala- | 100 | 1801.9 | 1801.7 | R8-S5 |
| | | Ala-Phe-Ser-S5-His-Tyr-Gln-NH2 | | | | stapled |
| CTNNB1 | FP07863 | Ac-Val-nLeu-Gln-R8-Tyr-Glu-Ala- | 101 | 1785.0 | 1784.8 | R8-S5 |
| | | Ala-Phe-Ile-S5-His-Tyr-Val-NH2 | | | | stapled |
| CTNNB1 | FP07864 | Ac-Val-nLeu-Gln-R8-Tyr-Glu-Ala- | 101 | 1785.0 | 1784.8 | R8-S5 |
| | | Ala-Phe-Ile-S5-His-Tyr-Val-NH2 | | | | stapled |
| CTNNB1 | FP07867 | Ac-Val-nLeu-Glu-R8-Tyr-Gln-Ala- | 102 | 1785.0 | 1784.8 | R8-S5 |
| | | Ala-Phe-Ile-S5-His-Tyr-Val-NH2 | | | | stapled |
| CTNNB1 | FP07868 | Ac-Val-nLeu-Glu-R8-Tyr-Gln-Ala- | 102 | 1785.0 | 1784.8 | R8-S5 |
| | | Ala-Phe-Ile-S5-His-Tyr-Val-NH2 | | | | stapled |
| CTNNB1 | FP07871 | Ac-Val-nLeu-Gln-R8-Tyr-Gln-Ala- | 103 | 1784.0 | 1783.8 | R8-S5 |
| | | Ala-Phe-Ile-S5-His-Tyr-Val-NH2 | | | | stapled |
| CTNNB1 | FP07872 | Ac-Val-nLeu-Gln-R8-Tyr-Gln-Ala- | 103 | 1784.0 | 1783.8 | R8-S5 |
| | | Ala-Phe-Ile-S5-His-Tyr-Val-NH2 | | | | stapled |
| CTNNB1 | FP08011 | Ac-Ile-Ile-His-R8-Tyr-Glu-Ala- | 104 | 1847.0 | 1847.2 | R8-S5 |
| | | Ala-Phe-Phe-S5-Gln-Tyr-Ile-NH2 | | | | stapled |
| CTNNB1 | FP08012 | Ac-Ile-Ile-His-R8-Tyr-Glu-Ala- | 104 | 1847.0 | 1847.4 | R8-S5 |
| | | Ala-Phe-Phe-S5-Gln-Tyr-Ile-NH2 | | | | stapled |
| CTNNB1 | FP08017 | Ac-Ile-Leu-Glu-R8-Tyr-Glu-Ala- | 105 | 1821.0 | 1821.1 | R8-S5 |
| | | Ala-Phe-Glu-S5-Gln-Tyr-nLeu-NH2 | | | | stapled |
| CTNNB1 | FP08018 | Ac-Ile-Leu-Glu-R8-Tyr-Glu-Ala- | 105 | 1821.0 | 1821.1 | R8-S5 |
| | | Ala-Phe-Glu-S5-Gln-Tyr-nLeu-NH2 | | | | stapled |
| CTNNB1 | FP08023 | Ac-Ile-nLeu-Ala-R8-Tyr-Gln-Ala- | 106 | 1820.0 | 1820.1 | R8-S5 |
| | | Ala-Phe-Trp-S5-Gln-Tyr-Asn-NH2 | | | | stapled |
| CTNNB1 | FP08024 | Ac-Ile-nLeu-Ala-R8-Tyr-Gln-Ala- | 106 | 1820.0 | 1820.1 | R8-S5 |
| | | Ala-Phe-Trp-S5-Gln-Tyr-Asn-NH2 | | | | stapled |
| CTNNB1 | FP08029 | Ac-Ile-Leu-Thr-R8-Tyr-Glu-Ala- | 107 | 1779.9 | 1780.1 | R8-S5 |
| | | Ala-Phe-Thr-S5-Gln-Tyr-Gln-NH2 | | | | stapled |
| CTNNB1 | FP08030 | Ac-Ile-Leu-Thr-R8-Tyr-Glu-Ala- | 107 | 1779.9 | 1780.1 | R8-S5 |
| | | Ala-Phe-Thr-S5-Gln-Tyr-Gln-NH2 | | | | stapled |

### Example 2. Technologies for Screening Stapled Peptide Collections

In some embodiments, the present disclosure provides technologies for identifying stapled peptides that can bind to targets of interest using stapled peptide collections. Among other things, the present disclosure provides a naive screening library. In some embodiments, a library comprises a collection of cysteine-stapled peptides, wherein the collection comprises random amino acid residues at one or more positions. In some embodiments, various stapled peptides in the library comprise alpha-helical structures. In one design, cysteine-stapled peptides are displayed as N-terminal fusions to a pIII protein on M13 bacteriophage. In some embodiments, stapled peptides are designed to contain one or more or all of the following features: a) a cap containing a proline residue at the N-terminus, or a N-terminal cap containing N-DPAA-C(SEQ ID NO: 20) sequence; b) two amino acid residues suitable for stapling, e.g., cysteine residues, placed at i,i+7 positions; c) one or more, e.g., three, randomized residues flanking each amino acid residue suitable for stapling, e.g., cysteine; d) a pair of randomized residues towards the inside of the stapled section between the two stapling residues, two alanine residues at the center of the stapled section (i,i+3 and i,i+4, numbered from the first amino acid residue suitable for stapling, e.g., cysteine) (without the intention to be limited by theory, in some embodiments, to minimize steric hindrance with the staple); and e) a short (8-residue) glycine-rich linker between the last randomized residue and the N-terminus of the pIII coat protein. Those skilled in the art will appreciate that if other technologies are utilized to generate a collection, a linker to a pIII coated protein may not be needed.

In some embodiments, a randomized peptide library was achieved by synthesizing and using degenerate phage library primers using a mix, e.g., an equimolar mix, of trimer phosphoroamidites that corresponded to various amino acid residues, for example, in some embodiments 16 of the 20 naturally occurring amino acids: alanine, arginine, asparagine, aspartic acid, glutamine, glutamic acid, histidine, isoleucine, leucine, methionine, phenyalanine, serine, threonine, tryptophan, tyrosine, and valine. In some embodiments, a X residue, e.g., X³¹, X², X³, etc., is selected from alanine, arginine, asparagine, aspartic acid, glutamine, glutamic acid, histidine, isoleucine, leucine, methionine, phenyalanine, serine, threonine, tryptophan, tyrosine, and valine. In some embodiments, cysteine and lysine were omitted to avoid intramolecular competition with the crosslinking between the two fixed stapling cysteine residues, and proline and glycine were omitted to avoid destabilization of the alpha-helical structure. In one embodiment, 10 randomized residues corresponds to a theoretical diversity of 16¹⁰≈10¹² possible sequences. In some embodiments, the number of unique sequences after transformation and amplification of such library was estimated to be approximately 10⁸ library members, in which case the constructed library explores roughly 0.01% of a theoretical sequence space of about 10¹² possible sequences.

Phage display has been reported to be performed by panning a library against a protein target and then eluting and amplifying the bound phage by infection of E. coli, followed by additional rounds of selection and amplification. Among other things, the present disclosure recognizes that while this approach can enable the detection of weak or initially low-abundance binders, it is not practical to perform multiple selections and enrichments on dozens or hundreds of selections in parallel, and/or it inherently prevents quantitative comparisons between different selections after the first round due to a number of factors. For example, in some embodiments, input phage libraries are no longer identical and/or there can be biases in elution efficiency, in growth, and/or infection rates.

In some embodiments, the present disclosure describes technologies for screening stapled peptide libraries. In some embodiments the present disclosure utilized a “single-round” approach wherein a library of stapled peptides, e.g., displayed on phages, are incubated with one or more protein targets. In some embodiments, NGS is utilized to identify and/or quantify stapled peptides that can bind to targets of interest. In some embodiments, individual protein targets have been immobilized, e.g., on magnetic beads, followed by protein target isolation, e.g., bead isolation and washing, and then identification of stapled peptides bound to target proteins. In some embodiments, after bead isolation and washing, direct boiling, PCR amplification, and next-generation sequencing (NGS) of phage DNA were performed, followed by hierarchical statistical clustering. In some embodiments, one or more phage screens (e.g., 48 or 96) are run in parallel, e.g., in multi-well plates, against a range of targets of interest, e.g., protein targets. In some embodiments, each target, e.g., each target in a well, receives approximately multiple, e.g., 100, copies of each library member, e.g., of each of the 10 library members in a library (10¹⁰ phage total). In some embodiments, provided technologies allow results of a target to be compared to others. In some embodiments, following target binding and washes, phage DNA is directly extracted, amplified and sequenced. In some embodiments, following the binding and washing steps, but prior to boiling and PCR, a defined quantity of a known sequence, e.g., phage sequence was spiked into each well, which can enable the normalization of NGS counts in each well, e.g., by dividing the number of reads for each library member by the number of reads for the spike-in, then multiplying the result by the number of spike-in copies that were originally added. In some embodiments, NGS counts in each well are normalized by dividing the number of reads for each library member by the number of reads for the spike-in, then multiplying the result by the number of spike-in copies that were originally added. In some embodiments, binding of each of the 10i library members is expressed in terms of eluted phage particles rather than as sequence reads.

In some embodiments, approximately the same number of phage particles were added to each well. In some embodiments, a quantitative comparison among one or more samples, for all library members can be performed, allowing stapled peptides to be selected for specific criteria in silico rather than by having to perform actual enrichment or depletion experiments. In some embodiments, non-specific or bead binders can be flagged by screening multiple proteins as well as blank beads, since such binders will appear in most or all samples, whereas highly specific binders will appear only in samples corresponding to one or more desired targets. In some embodiments, screens can be performed to search for stapled peptides with specific properties, such as their ability to compete with a known interaction or function of a target. In some embodiments, competition for binding sites can be assessed by screening a target in both the presence and absence of a binding partner or ligand, and searching the screening data for stapled peptides whose phage binding is affected when the partner or ligand is present. In some embodiments, these screens occur in vitro. In some embodiments, one or more or dozens or even hundreds of screens can be run in parallel. Among other things, the provided technologies can provide considerable flexibility in screen design and conditions. In some embodiments, screens were performed with a wide range of small molecule, peptidic, and protein binding partners. In some embodiments, proteins with different sequences (e.g., from different species), modifications, forms (with or without ligands, monomeric or polymeric forms, with or without binding partners, etc.), etc., can be compared. In some embodiments, proteins with or without a modification are compared. In some embodiments, phosphorylated vs. non-phosphorylated, proteins were compared. In some embodiments, glycosylated vs. non-glycosylated proteins were compared. In some embodiments, apo vs. ligand-bound proteins were compared. In some embodiments, mouse vs. human proteins were compared. In some embodiments, monomeric vs. multimeric proteins were compared.

In some embodiments, certain stapled peptides are selected based on their target-binding and selectivity properties. In some embodiments, certain stapled peptides are grouped into families of related sequences using hierarchical clustering. In some embodiments, it was observed certain positions appear to be highly conserved (enriched positions; the conserved amino acid residues, enriched amino acid residues). In some embodiments, only one or a small number of amino acids (enriched amino acid residues) are found at a conserved position across all members of a cluster. In some embodiments, these conserved positions are strongly predictive of which residues are forming direct binding interactions with their protein targets. In some embodiments, one or more conserved residues directly interact with protein targets. In some embodiments, direct interactions can and have been confirmed using suitable technologies, e.g., X-ray co-crystal structures of stapled peptides and their binding proteins. In some embodiments, conserved residues can be used to define a conserved pharmacophore and/or predict structure-activity relationships for a cluster.

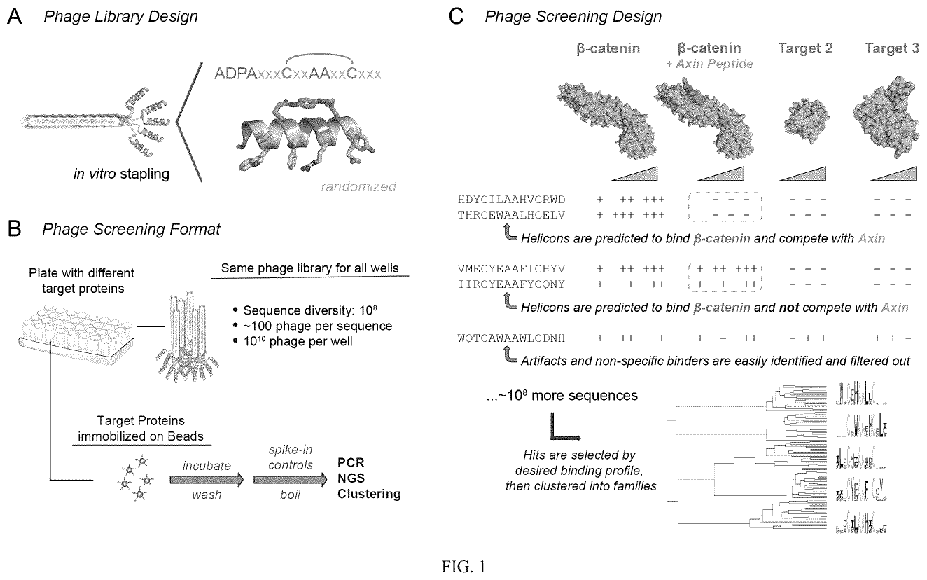

Certain useful technologies are described in FIG. 1 as examples. Binders for many targets of interest have been identified; certain examples are presented below.

### Example 3. Stapled Peptides Binding Beta-Catenin at Various Binding Sites

In some embodiments, provided technologies were utilized to discover stapled peptides that bind to therapeutic targets. In some embodiments, beta-catenin was screened against stapled peptide libraries. In some embodiments, the present disclosure provides stapled peptides that can bind beta-catenin at multiple binding sites. In some embodiments, the present disclosure provides stapled peptides that can modulate beta-catenin activities.

beta-Catenin is reported to be a key hub in the Wnt signaling pathway that is an attractive intracellular target for therapeutic intervention in many Wnt-driven cancers, such as those with APC or CTNNB1 mutations. beta-Catenin is reported to be engaged in numerous functional protein-protein interactions that have been difficult to target with traditional small molecules and biologics, but several examples of stapled peptide PPI inhibitors based on native binders have been described for beta-catenin and other “undruggable” targets. In one example, beta-catenin was screened at multiple concentrations, and in the presence and absence of an Axin-derived stapled peptide (which binds beta-catenin as a short alpha-helix) and the beta-catenin-binding domain of the TCF4 transcription factor. In some embodiments, certain stapled peptides that bind to beta-catenin competed with both Axin and TCF4. In some embodiments, certain stapled peptides that bind to beta-catenin competed with TCF4 but not with Axin.

In some embodiments, clusters of stapled peptides that competed with both Axin and TCF4 possessed different sequence patterns. In some embodiments, there were pairs of clusters that bore similar patterns of conserved residues, but that were shifted to different positions within the sequence, for example, Clusters C31 and C32 or C33 and C34. In some embodiments, members of a cluster may be binding beta-catenin in a similar way, but pharmacophores may be presented in different positions, or register, relative to the position of a staple. In some embodiments, such clusters are referred to as “shifts” of each other. In some embodiments, there were clusters whose patterns of conserved residues resembled the conserved residues in other clusters, , but were in the reverse orientation (N->C vs. C->N), for example, C32 and C33. In some embodiments, a similar binding mode is achieved from a peptide whose N- and C-termini have been inverted relative to another peptide, and side chains are able to form similar interactions, despite a different presentation angle from the main chain of the peptide. In some embodiments, analysis of co-crystal structures confirmed similar interactions despite inverted N- and C-termini. In some embodiments, conserved residues for several of these clusters, such as C31 or C32, bore close resemblance to the alpha-helical sequence in Axin that is known to bind beta-catenin.

In some embodiments, binding to beta-catenin, e.g., at the Axin site for certain stapled peptides were further assessed. In some embodiments, stapled peptides were synthesized and assessed in surface plasmon resonance (SPR) assays. In some embodiments, stapled peptides were synthesized and assessed in competition fluorescence polarization (FP) binding assays. In some embodiments, stapled peptides members of the C31-C35 clusters were synthesized and assessed in SPR and/or competition fluorescence polarization (FP) binding assays.

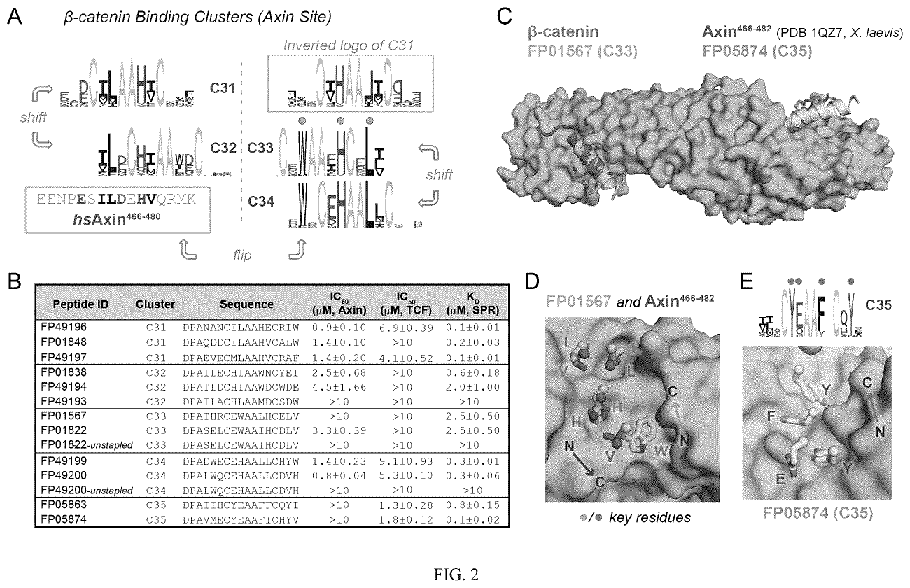

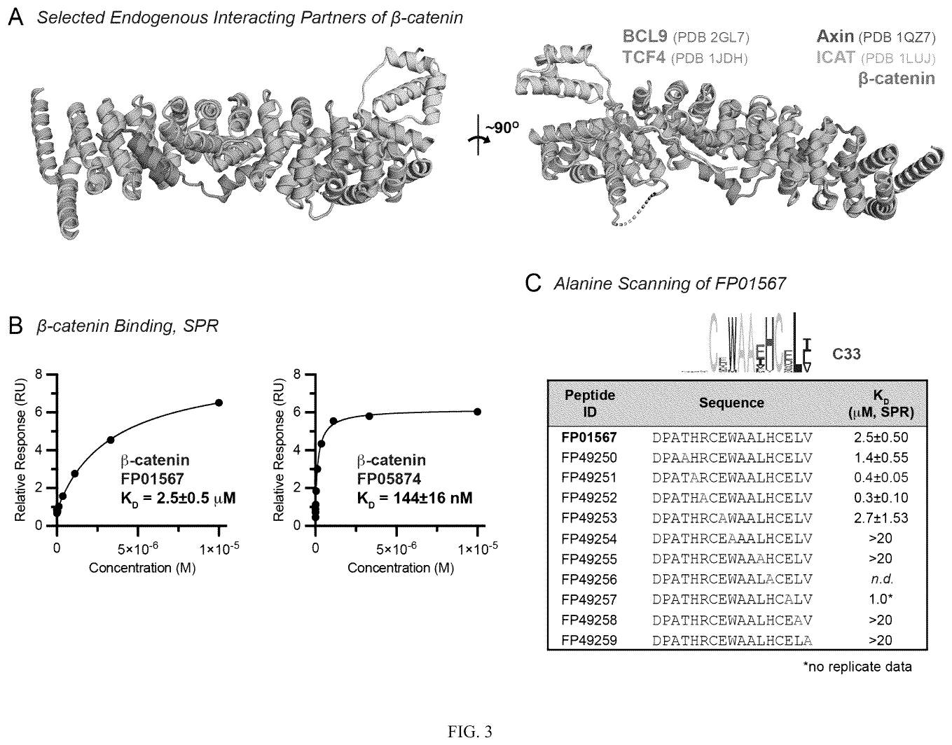

In one embodiment, SPR and competition FP data demonstrated binding to beta-catenin, as well as competition with both an Axin-derived Helicon and the full beta-catenin-binding domain of the TCF4 transcription factor. In some embodiment, SPR and competition FP data is consistent with the predicted binding behavior based on screens against beta-catenin. In some embodiments, direct interactions can and have been confirmed using suitable technologies, e.g., X-ray co-crystal structures of stapled peptides and their binding proteins. In some embodiments, beta-catenin binding site of stapled peptides from C33 cluster, e.g. FP01567, that was predicted to be a “flip” of the natural Axin sequence, was confirmed using suitable technologies, e.g. X-ray co-crystal structures and an alanine-screening analysis. In some embodiments, binding mode of FP01567 with beta-catenin was shown to be similar to binding mode of Axin but with an inverted/flipped N-to-C alpha-helical orientation and sidechains which were projected from a different direction. The crystal structure with FP01567, combined with an alanine-scanning analysis, further confirmed the importance of the conserved logo residues. Certain data are presented in FIG. 2, FIG. 3 and Table 2 as examples. Shift behavior and occasional flip behavior have also been observed for various other targets of interest. The Table below discloses SEQ ID NOS 901-905, 115, 906-907, 155, 908-909, 180, and 910-911, respectively, in order of appearance.

| | | | IC₅₀ | IC₅₀ | K_{D} |
|---|---|---|---|---|---|
| Peptide ID | Cluster | Sequence | (μM, Axin) | (μM, TCF) | (μM, SPR) |
| FP01838 | C31 | DPAILECHIAAWNCYEI | 1.8 | >10 | 0.4 |
| FP49194 | C31 | DPATLDCHIAAWDCWDE | 2.9 | >10 | 3 |
| FP49193 | C31 | DPAILACHLAAMDCSDW | >10 | >10 | n.d. |
| FP49196 | C32 | DPANANCILAAHECRIW | 1 | >10 | 0.1 |
| FP01848 | C32 | DPAQDDCILAAHVCALW | 1.3 | >10 | 0.14 |
| FP01848-unstapled | C32 | DPAQDDCILAAHVCALW | N.D. | >10 | >10 |
| FP01567 | C33 | DPATHRCEWAALHCELV | 7.3 | >10 | 3 |
| FP01822 | C33 | DPASELCEWAAIHCDLV | 3.7 | >10 | 3 |
| FP01822-unstapled | C33 | DPASELCEWAAHCDLV | >10 | >10 | >10 |
| FP49199 | C34 | DPADWECEHAALLCHYW | 1.1 | 8.1 | 0.3 |
| FP49200 | C34 | DPALWQCEHAALLCDVH | 0.78 | 5.3 | 0.4 |
| FP49200-unstapled | C34 | DPALWQCEHAALLCDVH | >10 | >10 | >10 |
| FP05863 | C35 | DPAIIHCYEAAFFCQYI | >10 | 1.0 | 0.7 |
| FP05874 | C35 | DPAVMECYEAAFICHYV | >10 | 1.5 | 0.15 |

In some embodiments, stapled peptides compete with TCF, but not Axin. In some embodiments, biochemical assays performed on members of Cluster C35 confirmed competition in vitro with TCF but not Axin. In some embodiments, co-crystal structures of member of C35 cluster, e.g., FP05877, indicated direct interactions with beta-catenin at a similar site as the naturally occurring beta-catenin binder ICAT. Certain results are presented in Table 2 as examples.

In some embodiments, cysteine staples are replaced with other types of staples. In some embodiments, Helicons with non-cysteine staples maintained binding to beta-catenin while also demonstrating cell entry, e.g., as assessed by mass spectrometry quantification of intact peptide in cells after treatment and washing. Certain results are presented in Table 2 as examples.

TABLE 2
Certain data as examples.

| | | | | | β-catenin | CellTiter- |
|---|---|---|---|---|---|---|
| | | | | Helicon | Binding | Glo fold |
| | | Parent | SEQ | in cellsª | IC₅₀^{b} (TCF | change^{c} |
| FOG ID | Cluster | peptide sequence | ID NO: | ± SD (%) | probe, uM) | ± SD |
| FP47839 | (Assay | 5FAM-SAH-p53-8^{d} | | 2.1 ± 0.1 | | 1.3 ± 0.01 |
| | Positive | | | | | |
| | Control) | | | | | |
| FP01413 | (Assay | ATSP-7041^{d} | | 3.7 ± 0.4 | | 1.2 ± 0.07 |
| | Positive | | | | | |
| | Control) | | | | | |
| FP47831 | (Assay | 5FAM-PKCI^{d} | | 0.0 ± 0.0 | | 1.1 ± 0.02 |
| | Negative | | | | | |
| | Control) | | | | | |
| FP47842 | (Assay | 5FAM-D-Myc peptide^{d} | | 0.0 ± 0.0 | | 1.2 ± 0.01 |
| | Negative | | | | | |
| | Control) | | | | | |
| FP47843 | (Assay | 5FAM-D-HA peptide^{d} | | 0.0 ± 0.0 | | 1.2 ± 0.02 |
| | Negative | | | | | |
| | Control) | | | | | |
| FP07855 | C35 | DPAVMECYEAAFICHYV | 108 | 16.4 ± 0.6 | 4.0 ± 0.1 | 1.2 ± 0.10 |
| | (CTNNB1) | | | | | |
| FP07856 | C35 | DPAVMECYEAAFICHYV | 108 | 15.5 ± 0.9 | 6.6 ± 0.9 | 1.3 ± 0.12 |
| | (CTNNB1) | | | | | |
| FP07859 | C35 | DPAILQCYEAAFSCHYQ | 109 | 0.1 ± 0.0 | >10 | 1.2 ± 0.02 |
| | (CTNNB1) | | | | | |
| FP07860 | C35 | DPAILQCYEAAFSCHYQ | 109 | 0.3 ± 0.0 | >10 | 1.2 ± 0.03 |
| | (CTNNB1) | | | | | |
| FP07863 | C35 | DPAVMECYEAAFICHYV | 108 | 23.5 ± 6.5 | 8.7 ± 1.6 | 1.2 ± 0.02 |
| | (CTNNB1) | | | | | |
| FP07864 | C35 | DPAVMECYEAAFICHYV | 108 | 18.5 ± 11.4 | 7.6 ± 1.1 | 1.1 ± 0.05 |
| | (CTNNB1) | | | | | |
| FP07867 | C35 | DPAVMECYEAAFICHYV | 108 | 8.6 ± 0.4 | >10 | 1.2 ± 0.03 |
| | (CTNNB1) | | | | | |
| FP07868 | C35 | DPAVMECYEAAFICHYV | 108 | 10.3 ± 1.4 | >10 | 1.2 ± 0.10 |
| | (CTNNB1) | | | | | |
| FP07871 | C35 | DPAVMECYEAAFICHYV | 108 | 7.0 ± 0.3 | >10 | 1.2 ± 0.03 |
| | (CTNNB1) | | | | | |
| FP07872 | C35 | DPAVMECYEAAFICHYV | 108 | 11.7 ± 1.6 | >10 | 1.1 ± 0.02 |
| | (CTNNB1) | | | | | |
| FP08011 | C35 | DPAIIHCYEAAFFCQYI | 110 | 6.5 ± 0.5 | >10 | 1.2 ± 0.01 |
| | (CTNNB1) | | | | | |
| FP08012 | C35 | DPAIIHCYEAAFFCQYI | 110 | 5.4 ± 0.9 | >10 | 1.1 ± 0.04 |
| | (CTNNB1) | | | | | |
| FP08017 | C35 | DPAILECYEAAFECQYM | 111 | 0.6 ± 0.1 | >10 | 1.2 ± 0.01 |
| | (CTNNB1) | | | | | |
| FP08018 | C35 | DPAILECYEAAFECQYM | 111 | 1.0 ± 0.1 | >10 | 1.1 ± 0.05 |
| | (CTNNB1) | | | | | |
| FP08023 | C35 | \|DPAIMACYQAAFWCQYN | 112 | 3.0 ± 0.3 | >10 | 1.2 ± 0.03 |
| | (CTNNB1) | | | | | |
| FP08024 | C35 | \|DPAIMACYQAAFWCQYN | 112 | 6.5 ± 0.5 | >10 | 1.2 ± 0.00 |
| | (CTNNB1) | | | | | |
| FP08029 | C35 | DPAILTCYEAAFTCQYQ | 113 | 0.2 ± 0.0 | 8.0 ± 1.1 | 1.1 ± 0.03 |
| | (CTNNB1) | | | | | |
| FP08030 | C35 | DPAILTCYEAAFTCQYQ | 113 | 0.3 ± 0.0 | >10 | 1.2 ± .02 |
| | (CTNNB1) | | | | | |

^{a}The percentage of Helicons in cells after treatment and wash. n = 2; data are presented as mean ± SD.
^{b}In vitro assay for competition with beta-catenin-binding TCF peptide.
^{c}Measure of ATP levels, reflective of cell health.
^{d}Full sequences of control peptides can be found in Table 1.

Stapled peptides of certain beta-catenin binding clusters are presented below as examples.

| Cluster C31 | | Target Concentration | | | | | | | | | | Competitors | |
|---|---|---|---|---|---|---|---|---|---|---|---|---|---|
| Sequence | SEQ ID NO | 10 | 30 | 100 | 300 | 1000 | 1000 | BLANK | BLANK | BLANK | BLANK | Axin_FP0597c_300 | TCF4_300 |
| DPAEHDCMLAAHFCRHF | 114 | 8 | 55 | 242 | 194 | 337 | 260 | 0 | 2 | 0 | 0 | 0 | 36 |
| DPAQDDCILAAHVCALW | 115 | 44 | 161 | 560 | 800 | 809 | 743 | 0 | 3 | 0 | 0 | 0 | 206 |
| DPAVEDCVLAAHVCAWF | 116 | 0 | 2 | 47 | 39 | 49 | 73 | 0 | 0 | 0 | 0 | 0 | 0 |
| DPAEAECILAAHICNIS | 117 | 10 | 0 | 37 | 39 | 51 | 52 | 0 | 0 | 0 | 0 | 0 | 0 |
| DPAEVDCIMAAHICVWD | 118 | 0 | 0 | 31 | 42 | 40 | 51 | 0 | 0 | 0 | 0 | 4 | 0 |
| DPADNECILAAHICTLA | 119 | 0 | 5 | 24 | 36 | 45 | 30 | 0 | 0 | 0 | 0 | 4 | 0 |
| DPAQQDCTLAAHVCRVF | 120 | 0 | 0 | 19 | 21 | 70 | 11 | 0 | 0 | 0 | 0 | 0 | 0 |
| DPAEADCMMAAHICSNI | 121 | 0 | 0 | 21 | 15 | 46 | 22 | 0 | 0 | 0 | 0 | 0 | 0 |
| DPADYECVIAAQVCQHF | 122 | 0 | 1 | 11 | 18 | 19 | 14 | 0 | 4 | 0 | 0 | 0 | 15 |
| DPANANCILAAHECRIW | 123 | 87 | 199 | 501 | 899 | 945 | 680 | 0 | 2 | 0 | 0 | 25 | 173 |
| DPAFESCFLAAHICYWE | 124 | 2 | 0 | 10 | 7 | 18 | 16 | 0 | 0 | 0 | 0 | 0 | 0 |
| DPAEAICILAAHECWWD | 125 | 19 | 38 | 139 | 235 | 322 | 228 | 0 | 0 | 0 | 0 | 0 | 28 |
| DPAEVECMLAAHVCRAF | 126 | 8 | 26 | 67 | 109 | 142 | 130 | 0 | 0 | 0 | 0 | 0 | 1 |
| DPADYDCILAAHICTNI | 127 | 2 | 18 | 70 | 147 | 118 | 122 | 0 | 0 | 0 | 0 | 19 | 0 |
| DPADTECILAAHFCHYF | 128 | 0 | 1 | 39 | 81 | 78 | 49 | 0 | 2 | 0 | 0 | 0 | 0 |
| DPAVDECIIAAHICHIW | 129 | 0 | 4 | 21 | 44 | 31 | 66 | 0 | 0 | 0 | 0 | 0 | 0 |
| DPAESDCILAAYICYVE | 130 | 0 | 1 | 22 | 51 | 69 | 40 | 0 | 2 | 0 | 0 | 0 | 0 |
| DPADEDCILAAHVCRHF | 131 | 0 | 6 | 11 | 29 | 25 | 21 | 0 | 0 | 0 | 0 | 0 | 1 |
| DPAADDCVLAAHVCAYF | 132 | 8 | 46 | 144 | 397 | 407 | 343 | 7 | 1 | 0 | 0 | 0 | 0 |
| DPARHECILAAHICEWE | 133 | 41 | 151 | 612 | 1704 | 1683 | 1509 | 1 | 2 | 2 | 0 | 2 | 144 |
| Cluster C32 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAILDCQIAAIECEFA | 134 | 0 | 22 | 76 | 68 | 139 | 56 | 0 | 0 | 0 | 1 | 0 | 3 |
| DPAILDCHIAAIQCFHD | 135 | 2 | 1 | 6 | 8 | 1 | 44 | 0 | 0 | 0 | 3 | 0 | 0 |
| Cluster C32 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAIFNCTMAALECTQI | 136 | 0 | 0 | 2 | 3 | 19 | 0 | 0 | 0 | 0 | 0 | 0 | 0 |
| DPAILECHIAAWNCYEI | 137 | 0 | 2 | 50 | 70 | 57 | 97 | 0 | 0 | 0 | 0 | 0 | 1 |
| DPAHLMCHYAAWDCRDE | 138 | 0 | 0 | 11 | 13 | 12 | 23 | 0 | 0 | 0 | 0 | 0 | 0 |
| DPAILDCQLAAMDCSTW | 139 | 0 | 6 | 22 | 21 | 25 | 55 | 0 | 0 | 0 | 0 | 15 | 0 |
| DPAFLTCHIAAWACSEQ | 140 | 0 | 1 | 21 | 15 | 52 | 25 | 0 | 2 | 1 | 0 | 0 | 0 |
| DPAFLDCHIAAWTCNEF | 141 | 0 | 1 | 10 | 15 | 28 | 15 | 0 | 0 | 0 | 0 | 0 | 0 |
| DPAILDCQLAAIDCVYT | 142 | 3 | 21 | 67 | 168 | 201 | 114 | 0 | 0 | 0 | 0 | 0 | 9 |
| DPAILDCSIAAIDCAYS | 143 | 0 | 0 | 16 | 42 | 39 | 29 | 0 | 0 | 0 | 0 | 0 | 0 |
| DPAILECHMAAFECYIE | 144 | 8 | 2 | 20 | 59 | 86 | 41 | 0 | 0 | 0 | 0 | 0 | 0 |
| DPAVFECSLAAIDCLQI | 145 | 0 | 0 | 10 | 28 | 10 | 81 | 0 | 0 | 0 | 0 | 4 | 0 |
| DPAVLECHIAAWECRMD | 146 | 0 | 0 | 24 | 83 | 91 | 45 | 0 | 0 | 0 | 0 | 0 | 0 |
| DPAILECHVAALDCQMW | 147 | 0 | 2 | 40 | 55 | 98 | 81 | 0 | 2 | 0 | 0 | 0 | 0 |
| DPAILNCHMAAWDCNFY | 148 | 0 | 7 | 14 | 57 | 37 | 59 | 0 | 0 | 1 | 0 | 0 | 0 |
| DPATLDCHIAAWDCWDE | 149 | 14 | 10 | 42 | 104 | 176 | 104 | 0 | 0 | 0 | 0 | 0 | 0 |
| DPAILACHLAAMDCSDW | 150 | 0 | 1 | 11 | 98 | 103 | 83 | 0 | 0 | 0 | 0 | 0 | 0 |
| DPAVLECHIAAWECNHT | 151 | 0 | 1 | 22 | 67 | 140 | 55 | 0 | 0 | 0 | 0 | 0 | 0 |
| DPAVLDCHIAALDCHAH | 152 | 0 | 1 | 0 | 10 | 39 | 3 | 0 | 0 | 0 | 0 | 0 | 0 |
| DPAILNCHVAAWNCHSF | 153 | 0 | 0 | 1 | 8 | 4 | 23 | 0 | 0 | 0 | 0 | 0 | 0 |
| Cluster C33 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAELACEWAAIHCELV | 154 | 0 | 2 | 41 | 94 | 69 | 75 | 0 | 0 | 0 | 0 | 0 | 17 |
| DPASELCEWAAIHCDLV | 155 | 19 | 41 | 163 | 495 | 605 | 424 | 1 | 2 | 3 | 1 | 0 | 3 |
| DPADDFCMWAAEHCELL | 156 | 10 | 12 | 81 | 226 | 273 | 227 | 0 | 0 | 1 | 0 | 0 | 1 |
| DPAWQNCTWAAFHCELL | 157 | 0 | 10 | 52 | 194 | 158 | 163 | 0 | 1 | 0 | 0 | 0 | 39 |
| DPAQNICEWAALHCDLI | 158 | 0 | 13 | 37 | 109 | 177 | 82 | 0 | 1 | 0 | 0 | 0 | 10 |
| Cluster C33 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAEFYCEMAAVHCELL | 159 | 2 | 1 | 17 | 99 | 61 | 107 | 0 | 0 | 0 | 0 | 0 | 0 |
| DPAVHRCFWAAEHCDLI | 160 | 0 | 2 | 11 | 86 | 95 | 62 | 0 | 2 | 0 | 0 | 0 | 0 |
| DPAWILCDWAAMHCELV | 161 | 0 | 0 | 2 | 3 | 1 | 18 | 0 | 0 | 0 | 0 | 0 | 0 |
| DPAMIQCQWAAEHCELI | 162 | 0 | 0 | 4 | 33 | 19 | 42 | 0 | 1 | 0 | 0 | 0 | 0 |
| DPAIADCDWAAEHCDLL | 163 | 0 | 6 | 118 | 266 | 325 | 417 | 0 | 1 | 4 | 1 | 0 | 1 |
| DPAVQDCEWAAIYCELI | 164 | 0 | 9 | 10 | 158 | 104 | 157 | 0 | 1 | 0 | 1 | 0 | 12 |
| DPAWSDCIWAAEHCQLI | 165 | 0 | 3 | 5 | 127 | 109 | 85 | 0 | 1 | 0 | 0 | 0 | 0 |
| DPAMSQCNWAAVHCQLL | 166 | 6 | 6 | 6 | 13 | 15 | 23 | 0 | 0 | 0 | 0 | 0 | 0 |
| DPANVECEWAAIHCSLV | 167 | 13 | 2 | 19 | 158 | 164 | 187 | 0 | 0 | 0 | 0 | 0 | 3 |
| DPAQSDCDWAAEHCNLI | 168 | 0 | 1 | 0 | 23 | 9 | 57 | 0 | 0 | 0 | 0 | 2 | 1 |
| DPAAQRCTWAAIHCELI | 169 | 0 | 0 | 7 | 39 | 25 | 93 | 0 | 0 | 2 | 0 | 0 | 1 |
| DPAYTYCQWAAEHCSLL | 170 | 5 | 5 | 3 | 88 | 115 | 137 | 2 | 2 | 1 | 2 | 0 | 0 |
| DPAHWSCEWAAEHCRLI | 171 | 0 | 1 | 0 | 31 | 19 | 64 | 0 | 0 | 0 | 0 | 0 | 0 |
| DPAENTCSWAAEHCALV | 172 | 0 | 0 | 0 | 26 | 28 | 31 | 0 | 0 | 0 | 0 | 0 | 0 |
| DPATWHCDWAAEHCELL | 173 | 2 | 10 | 81 | 147 | 203 | 280 | 0 | 0 | 0 | 0 | 0 | 3 |
| Cluster C34 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPALWNCDHAALLCDHQ | 174 | 0 | 0 | 10 | 8 | 13 | 10 | 0 | 0 | 0 | 0 | 0 | 0 |
| DPAEWDCMHAALICEVH | 175 | 0 | 10 | 55 | 54 | 67 | 89 | 0 | 0 | 0 | 0 | 0 | 0 |
| DPASWRCEHAALNCQID | 176 | 0 | 6 | 20 | 18 | 21 | 57 | 0 | 0 | 0 | 0 | 0 | 0 |
| DPAWWQCDHAALICIAE | 177 | 28 | 127 | 422 | 637 | 830 | 731 | 0 | 1 | 2 | 0 | 32 | 113 |
| DPAEWQCEHAALLCEQQ | 178 | 78 | 201 | 907 | 1681 | 1740 | 1609 | 0 | 2 | 0 | 6 | 36 | 154 |
| DPAQWACEHAALLCSMD | 179 | 6 | 35 | 226 | 419 | 326 | 432 | 0 | 1 | 0 | 0 | 0 | 25 |
| DPALWQCEHAALLCDVH | 180 | 13 | 27 | 122 | 158 | 246 | 246 | 0 | 2 | 1 | 0 | 0 | 0 |
| DPADWICEHAALTCQEF | 181 | 0 | 2 | 6 | 15 | 12 | 14 | 0 | 1 | 0 | 0 | 0 | 0 |
| Cluster C34 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAHWVCEHAALNCDQN | 182 | 0 | 2 | 47 | 96 | 104 | 118 | 0 | 1 | 0 | 0 | 0 | 0 |
| DPADWECEHAALLCHYW | 183 | 11 | 18 | 90 | 179 | 198 | 226 | 0 | 2 | 0 | 0 | 0 | 1 |
| DPAEWICEHAALLCSVL | 184 | 0 | 0 | 11 | 33 | 33 | 27 | 0 | 0 | 0 | 0 | 0 | 0 |
| DPAEWACEHAALTCWDQ | 185 | 6 | 24 | 148 | 448 | 431 | 449 | 1 | 4 | 0 | 0 | 0 | 33 |
| DPASWDCDHAALLCTLD | 186 | 3 | 6 | 86 | 257 | 283 | 238 | 0 | 0 | 0 | 1 | 0 | 0 |
| DPAQFYCEHAALLCRWE | 187 | 0 | 0 | 5 | 11 | 15 | 11 | 0 | 0 | 0 | 0 | 0 | 0 |
| DPAHWRCEHAALLCWYN | 188 | 13 | 6 | 51 | 132 | 140 | 149 | 0 | 0 | 2 | 0 | 0 | 0 |
| DPAEWYCEHAALVCTNH | 189 | 0 | 12 | 47 | 156 | 148 | 155 | 2 | 0 | 0 | 1 | 0 | 0 |
| DPAMWICEHAALNCEIR | 190 | 2 | 6 | 46 | 158 | 145 | 152 | 0 | 0 | 0 | 0 | 0 | 7 |
| DPAYWDCTHAALLCNLD | 191 | 3 | 14 | 152 | 42 | 543 | 409 | 0 | 1 | 2 | 0 | 0 | 19 |
| DPAHWHCEHAALLCASV | 192 | 2 | 15 | 60 | 231 | 177 | 234 | 1 | 0 | 0 | 0 | 0 | 1 |
| DPAYWVCEHAALLCTDY | 193 | 5 | 18 | 85 | 337 | 283 | 227 | 3 | 3 | 1 | 0 | 0 | 4 |
| Cluster C35 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAIIHCYEAAFFCQYI | 110 | 0 | 0 | 15 | 20 | 22 | 28 | 1 | 0 | 0 | 1 | 27 | 1 |
| DPAILECYEAAFECQYM | 111 | 7 | 8 | 20 | 49 | 41 | 39 | 0 | 0 | 0 | 0 | 87 | 0 |
| DPAIMACYQAAFWCQYN | 112 | 0 | 0 | 4 | 27 | 29 | 18 | 1 | 0 | 1 | 0 | 38 | 0 |
| DPATIRCYEAAFMCQYW | 194 | 0 | 0 | 3 | 34 | 21 | 38 | 0 | 0 | 0 | 0 | 23 | 0 |
| DPAILTCYEAAFTCQYQ | 113 | 0 | 7 | 18 | 71 | 69 | 105 | 0 | 1 | 0 | 0 | 107 | 0 |
| DPAIMACYEAAYLCDYI | 195 | 2 | 4 | 29 | 85 | 111 | 150 | 1 | 0 | 0 | 1 | 130 | 0 |
| DPAIIRCYEAAFYCQNY | 196 | 0 | 3 | 10 | 67 | 93 | 94 | 0 | 0 | 1 | 0 | 124 | 0 |
| DPAVIQCYQAAFHCDYQ | 197 | 0 | 0 | 0 | 13 | 12 | 30 | 0 | 0 | 3 | 0 | 14 | 0 |
| DPAEIICYEAAFLCQYQ | 198 | 0 | 0 | 0 | 15 | 38 | 26 | 0 | 0 | 0 | 0 | 40 | 0 |
| DPAALSCYQAAFHCQYY | 199 | 0 | 0 | 1 | 5 | 14 | 17 | 0 | 0 | 0 | 0 | 16 | 0 |
| DPARMTCYEAAFRCQYY | 200 | 0 | 2 | 2 | 3 | 11 | 9 | 0 | 0 | 0 | 0 | 4 | 0 |
| Cluster C35 | | Target Concentration | | | | | | | | | | Competitors | |
| Sequence | SEQ ID NO: | 10 | 30 | 100 | 300 | 1000 | 1000 | BLANK | BLANK | BLANK | BLANK | Axin_FP0597c_300 | TCF4_300 |
| DPAVVECYEAAYTCQYI | 201 | 0 | 0 | 0 | 0 | 0 | 13 | 0 | 0 | 0 | 0 | 0 | 0 |
| DPAVMECYEAAFICHYV | 108 | 0 | 27 | 16 | 79 | 51 | 74 | 0 | 0 | 0 | 0 | 120 | 0 |
| DPAILQCYEAAFSCHYQ | 109 | 0 | 9 | 40 | 54 | 39 | 42 | 0 | 0 | 0 | 0 | 47 | 0 |
| DPAVLQCYEAAFICEYM | 202 | 0 | 0 | 1 | 6 | 4 | 5 | 0 | 0 | 0 | 0 | 2 | 0 |
| DPAVMACYEAAFQCAYW | 203 | 0 | 0 | 0 | 4 | 12 | 0 | 0 | 0 | 1 | 0 | 36 | 0 |
| DPAVIACYDAAFVCEYF | 204 | 0 | 0 | 0 | 0 | 3 | 3 | 0 | 1 | 0 | 0 | 0 | 0 |
| DPAVFMCYEAAFECQYF | 205 | 0 | 0 | 0 | 1 | 2 | 7 | 0 | 2 | 1 | 0 | 13 | 0 |
| DPAIIHCYQAAFQCQYM | 206 | 0 | 0 | 0 | 0 | 7 | 0 | 0 | 0 | 0 | 0 | 0 | 0 |
| DPAMVTCYEAAYVCQYF | 207 | 0 | 0 | 0 | 0 | 7 | 0 | 0 | 0 | 0 | 0 | 0 | 0 |

### Example 4. Stapled Peptides Binding the E3 Ubiquitin Ligase RNF31 at Various Binding Sites

In some embodiments, the present disclosure provides technologies useful for identifying, characterizing, manufacturing, assessing, using, etc., stapled peptide binders to targets of interest, e.g., proteins, with few or no known alpha-helical binding sites. In some embodiments, the present disclosure provides stapled peptide agents that bind E3 ubiquitin ligase RNF31. In some embodiments, the present disclosure provides stapled peptide agents that modulate activities of RNF31. RNF31 is reported to be an integral component of the linear ubiquitin chain assembly complex (LUBAC). It is reported to be a 1,072-amino acid protein comprised of multiple folded and disordered regions. In some embodiments, two of the non-enzymatic folded domains of RNF31, the PUB and UBA domains, were screened, each at multiple concentrations and in the presence or absence of a known binding partner.

Screens for both UBA and PUB domains afforded multiple distinct clusters of binders, and several instances of “shift” clusters were observed. In some embodiments, most hits for the RNF31 PUB domain could be grouped into two families of related clusters, (C41-C43 and C44-C45), both of which competed with the natural binding partner Otulin, and both of which appeared to contain a critical tyrosine residue—also present in Otulin—but that had other dissimilar structure features. In some embodiments, X-ray co-crystal structures of member from a representative cluster of each family revealed that the two Helicons, FP06649 from Cluster C44 and FP06652 from C41, bound the same site in the protein surface, but that they engaged this site using completely different binding modes, with the critical tyrosine residues oriented differently. Among other other things, the present disclosure provides distinct binding solutions to a common site, e.g., for RNF31 and other targets of interest.

In some embodiments, binders to the RNF31 UBA domain were observed to compete with its partner Sharpin/SIPL1. In one embodiment, these binders could be grouped into at least two families. One of these families, comprised of Cluster C46 and C47, was characterized by a shared compact pharmacophore containing conserved leucine/phenylalanine, serine, valine, and tyrosine residues that was observed in two shifted registers. In some embodiments, biochemical characterization of members from C46 revealed that several stapled peptides bound with mid-nanomolar affinity. In some embodiments, analysis of a co-crystal structure of FP06655, from C46 cluster, in complex with the RNF31 UBA domain, revealed an large conformational rearrangement when compared to the co-crystal structure of the RNF31 UBA domain in complex with its natural binding partner Sharpin. In some embodiments, binding of FP06655 with this target involves a significant rotation of the bundle of three N-terminal a-helices of the RNF31 UBA domain to form a groove in which the conserved leucine/phenylalanine and valine residues of FP06655 engage one wall of the groove, and the conserved serine and tyrosine residues engage the opposite wall, thereby stabilizing the RNF31 UBA domain in this induced fit. In some embodiments, it was showed that Sharpin UBL indeed competes with 5FAM-labeled FP06655 in solution. In some embodiments, binding of stapled peptides to their targets lead to significant induced fits.

Stapled peptides of certain RNF31 binding clusters are presented below as examples.

| Cluster C41 | | Target Concentration | | | | | | | | | | | Competitor |
|---|---|---|---|---|---|---|---|---|---|---|---|---|---|
| Sequence | SEQ ID NO: | 10 | 30 | 100 | 300 | 1000 | 1000 | BLANK | BLANK | BLANK | BLANK | BLANK | FP3455_300 |
| DPAIVQCAWAALYCDMQ | 208 | 0 | 0 | 0 | 7 | 22 | 20 | 0 | 0 | 0 | 0 | 0 | 0 |
| DPALEDCQWAALYCDEY | 209 | 0 | 0 | 1 | 2 | 7 | 9 | 1 | 1 | 1 | 0 | 0 | 1 |
| DPAYEDCAWAALYCDMT | 210 | 0 | 0 | 0 | 10 | 70 | 64 | 0 | 0 | 0 | 0 | 0 | 0 |
| DPAYYTCDWAAIYCDYH | 211 | 0 | 0 | 0 | 1 | 6 | 6 | 0 | 1 | 0 | 0 | 0 | 0 |
| DPAYRDCDWAALYCDDF | 212 | 0 | 0 | 1 | 2 | 12 | 12 | 0 | 3 | 0 | 0 | 1 | 0 |
| DPAWTQCDWAALYCEDQ | 213 | 0 | 1 | 0 | 1 | 7 | 10 | 0 | 0 | 0 | 0 | 0 | 0 |
| DPAEDECDWAALYCDMQ | 214 | 0 | 0 | 0 | 1 | 7 | 6 | 0 | 0 | 0 | 0 | 0 | 0 |
| DPAYEDCADAAMYCDMD | 215 | 0 | 0 | 0 | 0 | 11 | 10 | 1 | 0 | 0 | 0 | 0 | 0 |
| DPAWTDCDWAALYCEFQ | 216 | 1 | 0 | 0 | 0 | 6 | 6 | 0 | 0 | 0 | 1 | 0 | 0 |
| DPAWTDCDWAALYCDES | 217 | 0 | 0 | 0 | 0 | 8 | 4 | 0 | 0 | 0 | 0 | 0 | 0 |
| DPALVTCTWAAIYCDDF | 218 | 1 | 1 | 2 | 1 | 4 | 10 | 0 | 1 | 0 | 2 | 0 | 0 |
| DPAYTLCSWAAMYCDSM | 219 | 0 | 1 | 0 | 0 | 5 | 6 | 0 | 1 | 1 | 4 | 2 | 2 |
| DPAYSSCSWAAIYCNWT | 220 | 1 | 1 | 5 | 2 | 7 | 4 | 1 | 1 | 4 | 2 | 1 | 1 |
| DPAADDCEWAALYCSFH | 221 | 2 | 0 | 0 | 3 | 3 | 8 | 2 | 9 | 0 | 2 | 3 | 0 |
| Cluster C42 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3455_300 |
| DPAWNDCLYAAETCISW | 222 | 1 | 1 | 1 | 3 | 11 | 1 | 2 | 1 | 1 | 0 | 0 | 0 |
| DPAWWNCLYAANTCVED | 223 | 0 | 1 | 0 | 5 | 7 | 5 | 1 | 2 | 1 | 1 | 1 | 1 |
| DPAQHECIYAAWDCDML | 224 | 1 | 1 | 1 | 5 | 6 | 8 | 3 | 2 | 0 | 1 | 0 | 5 |
| DPAWWDCMYAASHCDTV | 225 | 0 | 0 | 0 | 3 | 12 | 2 | 2 | 0 | 0 | 0 | 0 | 0 |
| DPAWMQCMYAAWDCNIE | 226 | 0 | 0 | 0 | 4 | 6 | 5 | 0 | 0 | 0 | 0 | 0 | 1 |
| DPAYWDCMYAAFYCDDI | 227 | 0 | 0 | 0 | 3 | 3 | 8 | 0 | 0 | 0 | 0 | 2 | 0 |
| DPAYWDCLYAAADCAFY | 228 | 0 | 0 | 0 | 4 | 8 | 5 | 2 | 0 | 0 | 0 | 0 | 0 |
| DPAYWDCLYAAWSCDIL | 229 | 0 | 1 | 0 | 4 | 9 | 7 | 1 | 1 | 1 | 0 | 0 | 1 |
| DPAWWSCMYAAWTCDDT | 230 | 1 | 10 | 0 | 7 | 14 | 15 | 0 | 2 | 2 | 1 | 1 | 2 |
| DPAWLDCIYAAYDCVTD | 231 | 1 | 1 | 1 | 3 | 6 | 4 | 1 | 1 | 1 | 0 | 2 | 1 |
| DPAYWQCIYAAWDCDQM | 232 | 0 | 0 | 1 | 4 | 13 | 6 | 1 | 2 | 2 | 1 | 0 | 4 |
| DPAWWNCLYAAYECNHA | 233 | 1 | 0 | 0 | 3 | 9 | 5 | 0 | 0 | 0 | 2 | 0 | 0 |
| DPALWDCLYAADACITS | 234 | 0 | 1 | 0 | 4 | 8 | 12 | 0 | 2 | 1 | 0 | 0 | 0 |
| DPAFWECMYAASDCDAS | 235 | 0 | 0 | 1 | 2 | 6 | 4 | 1 | 0 | 0 | 0 | 2 | 0 |
| DPAFWACLYAAEECVVD | 236 | 0 | 0 | 0 | 3 | 6 | 9 | 1 | 0 | 0 | 0 | 1 | 0 |
| DPAWMDCLYAAYDCQHM | 237 | 2 | 2 | 0 | 9 | 29 | 24 | 1 | 1 | 1 | 0 | 0 | 1 |
| DPAYWHCLYAAASCDDI | 238 | 0 | 0 | 0 | 2 | 8 | 4 | 0 | 3 | 0 | 0 | 2 | 0 |
| DPAEWDCLYAAFDCFAN | 239 | 2 | 2 | 0 | 3 | 15 | 7 | 0 | 0 | 0 | 0 | 0 | 0 |
| DPADWDCMYAAQDCFED | 240 | 0 | 0 | 1 | 1 | 11 | 1 | 0 | 0 | 0 | 0 | 0 | 0 |
| DPAWWNCLYAADECWSW | 241 | 0 | 0 | 0 | 3 | 13 | 6 | 0 | 0 | 1 | 0 | 0 | 0 |
| Cluster C43 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3455_300 |
| DPAYDWCMYAAHECVDQ | 242 | 0 | 0 | 0 | 3 | 3 | 8 | 0 | 0 | 0 | 1 | 0 | 1 |
| DPALDYCLYAAYDCMYA | 243 | 0 | 0 | 0 | 3 | 5 | 12 | 0 | 3 | 0 | 0 | 0 | 3 |
| DPATWWCLYAAEDCVNI | 244 | 0 | 1 | 0 | 2 | 6 | 6 | 0 | 0 | 0 | 0 | 4 | 0 |
| DPAYDWCLYAAYQCDQN | 245 | 0 | 0 | 0 | 6 | 13 | 40 | 0 | 0 | 0 | 0 | 0 | 0 |
| DPAWEWCLYAAAECIED | 246 | 0 | 0 | 0 | 2 | 12 | 5 | 0 | 0 | 0 | 0 | 0 | 0 |
| DPAEDWCMYAAWDCQME | 247 | 1 | 0 | 0 | 4 | 22 | 10 | 0 | 0 | 0 | 0 | 0 | 0 |
| DPAQDWCMYAAYECDAH | 248 | 0 | 0 | 0 | 2 | 7 | 9 | 0 | 0 | 2 | 1 | 1 | 0 |
| DPAWDYCMYAAYDCNHS | 249 | 2 | 0 | 1 | 8 | 46 | 29 | 0 | 1 | 0 | 0 | 1 | 2 |
| DPARDWCLYAAYDCYTA | 250 | 0 | 0 | 4 | 61 | 350 | 353 | 1 | 0 | 3 | 1 | 0 | 2 |
| DPAHDWCLYAAYDCRVY | 251 | 0 | 0 | 1 | 2 | 15 | 8 | 0 | 0 | 0 | 0 | 0 | 0 |
| DPAWEHCLYAAYDCTHE | 252 | 0 | 1 | 0 | 2 | 10 | 15 | 0 | 4 | 0 | 0 | 0 | 4 |
| DPAYDWCMYAAYNCVED | 253 | 2 | 1 | 1 | 6 | 41 | 38 | 0 | 0 | 0 | 0 | 0 | 0 |
| DPAWESCMYAAYDCDNE | 254 | 0 | 1 | 0 | 2 | 14 | 5 | 0 | 0 | 0 | 0 | 0 | 0 |
| DPAEQFCLYAAYDCYSE | 255 | 2 | 0 | 0 | 2 | 18 | 9 | 1 | 3 | 1 | 0 | 0 | 0 |
| DPAYDWCLYAAEYCHTD | 256 | 0 | 0 | 0 | 1 | 7 | 4 | 0 | 0 | 1 | 0 | 0 | 0 |
| DPAYDYCLYAAQDCVWW | 257 | 0 | 0 | 0 | 1 | 6 | 5 | 0 | 0 | 0 | 0 | 0 | 0 |
| DPAFDWCLYAADDCAET | 258 | 1 | 0 | 0 | 3 | 31 | 12 | 0 | 0 | 0 | 0 | 1 | 0 |
| DPAHTWCMYAAFDCFES | 259 | 1 | 0 | 0 | 1 | 9 | 4 | 1 | 2 | 2 | 0 | 0 | 0 |
| DPAYDYCLYAAYDCTQT | 260 | 0 | 0 | 1 | 5 | 48 | 38 | 1 | 0 | 0 | 1 | 0 | 0 |
| Cluster C44 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3455_300 |
| DPAQYQCDIAAQVCSTY | 261 | 0 | 0 | 0 | 4 | 12 | 10 | 0 | 0 | 0 | 0 | 0 | 0 |
| DPAFAQCEVAAMVCTTY | 262 | 0 | 0 | 0 | 2 | 5 | 6 | 0 | 0 | 0 | 0 | 0 | 0 |
| DPAIQECLLAAATCWTY | 263 | 0 | 0 | 0 | 2 | 8 | 4 | 0 | 0 | 0 | 0 | 0 | 0 |
| DPAHANCQLAADVCSTY | 264 | 0 | 0 | 0 | 13 | 49 | 33 | 1 | 0 | 0 | 0 | 0 | 0 |
| DPAWQECVIAALVCDTY | 265 | 0 | 0 | 0 | 3 | 13 | 8 | 0 | 0 | 1 | 4 | 0 | 0 |
| DPAIYQCSLAADICSTY | 266 | 1 | 3 | 3 | 3 | 7 | 8 | 2 | 2 | 0 | 0 | 0 | 1 |
| DPAIWECVSAAAVCMTY | 267 | 0 | 0 | 0 | 2 | 7 | 7 | 0 | 0 | 0 | 1 | 0 | 0 |
| DPAIQECYLAAQVCTTY | 268 | 0 | 0 | 0 | 4 | 14 | 13 | 0 | 0 | 0 | 0 | 0 | 0 |
| DPAAWECHIAAFVCSTY | 269 | 1 | 1 | 0 | 2 | 8 | 7 | 0 | 0 | 0 | 0 | 0 | 0 |
| DPAAEDCHIAAMVCMTY | 270 | 0 | 4 | 0 | 12 | 46 | 67 | 0 | 0 | 0 | 0 | 0 | 0 |
| DPAIRECMLAALVCSTY | 271 | 0 | 1 | 3 | 10 | 42 | 49 | 0 | 0 | 0 | 1 | 0 | 1 |
| DPAFTDCQLAAAVCMTY | 272 | 0 | 0 | 3 | 31 | 161 | 145 | 1 | 2 | 0 | 0 | 0 | 1 |
| DPAADQCDIAAFVCSTY | 273 | 0 | 0 | 0 | 4 | 26 | 17 | 0 | 0 | 0 | 0 | 0 | 0 |
| DPAFMDCVFAAAVCATY | 274 | 0 | 0 | 0 | 2 | 8 | 8 | 0 | 1 | 1 | 0 | 0 | 0 |
| DPALSHCTVAAMVCSTY | 275 | 1 | 1 | 1 | 11 | 96 | 69 | 0 | 0 | 2 | 0 | 0 | 1 |
| DPANFTCRVAADVCSTY | 276 | 0 | 0 | 3 | 3 | 26 | 12 | 0 | 0 | 1 | 0 | 0 | 0 |
| DPAIAQCMLAAQVCSTY | 277 | 2 | 0 | 0 | 5 | 54 | 34 | 1 | 0 | 0 | 0 | 0 | 0 |
| DPAMQDCQLAALVCTTY | 278 | 0 | 1 | 0 | 1 | 6 | 6 | 0 | 0 | 0 | 0 | 0 | 0 |
| DPAFHDCQFAAAVCSTY | 279 | 0 | 0 | 0 | 3 | 26 | 26 | 0 | 0 | 0 | 0 | 0 | 0 |
| DPAMIDCFLAAQVCATY | 280 | 0 | 0 | 0 | 1 | 6 | 6 | 0 | 0 | 0 | 0 | 0 | 0 |
| Cluster C45 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3455_300 |
| DPAFQFCVEAAVACSYY | 281 | 0 | 0 | 0 | 6 | 9 | 5 | 0 | 0 | 0 | 0 | 4 | 0 |
| DPAEDECQDAAILCSYN | 282 | 1 | 3 | 0 | 7 | 19 | 6 | 1 | 0 | 0 | 0 | 1 | 1 |
| DPALRDCLDAAIMCTYT | 283 | 0 | 1 | 0 | 4 | 5 | 7 | 0 | 0 | 0 | 0 | 0 | 0 |
| DPAQYQCWNAAMSCSYS | 284 | 1 | 1 | 3 | 3 | 7 | 4 | 1 | 0 | 0 | 3 | 2 | 0 |
| DPAQMECIDAAIMCSYT | 285 | 0 | 0 | 0 | 3 | 5 | 5 | 0 | 0 | 0 | 0 | 0 | 0 |
| DPAQDQCISAAVLCTYT | 286 | 0 | 0 | 0 | 22 | 44 | 37 | 0 | 0 | 1 | 0 | 0 | 0 |
| DPAQEACWDAAIMCTYR | 287 | 0 | 0 | 0 | 29 | 56 | 80 | 0 | 0 | 0 | 0 | 0 | 0 |
| DPAEEYCLEAAILCTYD | 288 | 0 | 0 | 1 | 14 | 44 | 32 | 0 | 0 | 0 | 0 | 0 | 0 |
| DPAMQECMDAAIMCTYA | 289 | 0 | 0 | 0 | 4 | 14 | 8 | 0 | 2 | 0 | 0 | 0 | 0 |
| DPAMEWCWDAAVFCTYS | 290 | 1 | 1 | 0 | 6 | 18 | 16 | 2 | 1 | 0 | 1 | 1 | 0 |
| DPAVVDCWDAAVQCTYY | 291 | 0 | 0 | 0 | 4 | 15 | 11 | 0 | 0 | 0 | 2 | 0 | 1 |
| DPAIDACYDAAVMCTYV | 292 | 0 | 0 | 0 | 6 | 27 | 15 | 0 | 0 | 0 | 0 | 4 | 0 |
| DPASHMCLEAAVMCTYD | 293 | 0 | 0 | 0 | 4 | 10 | 13 | 0 | 0 | 0 | 0 | 0 | 0 |
| DPATEACFDAAIECTYY | 294 | 0 | 0 | 0 | 2 | 7 | 5 | 0 | 0 | 0 | 0 | 0 | 0 |
| DPAYDLCLNAAVACTYV | 295 | 0 | 2 | 2 | 19 | 62 | 71 | 1 | 2 | 1 | 0 | 0 | 0 |
| DPAYSLCISAAVECTYY | 296 | 0 | 1 | 0 | 2 | 6 | 7 | 0 | 0 | 0 | 0 | 0 | 0 |
| DPARRECWDAAILCTYY | 297 | 0 | 0 | 1 | 12 | 40 | 44 | 0 | 0 | 1 | 0 | 0 | 0 |
| DPASEWCLDAAVLCTYS | 298 | 0 | 0 | 3 | 18 | 63 | 65 | 2 | 0 | 0 | 0 | 0 | 0 |
| DPALEICWDAAIQCSYV | 299 | 2 | 3 | 4 | 47 | 186 | 171 | 2 | 0 | 0 | 3 | 1 | 1 |
| DPAQEECFEAAVFCSYY | 300 | 2 | 0 | 2 | 7 | 36 | 20.49 | 0 | 0 | 0 | 0 | 1 | 0 |
| Cluster C46 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3456s_300 |
| DPAMQRCFSAAVYCAIS | 301 | 11 | 44 | 101 | 150 | 156 | 159 | 0 | 0 | 1 | 0 | 1 | 158 |
| DPAQMSCFSAAVYCRLF | 302 | 0 | 0 | 11 | 10 | 10 | 17 | 0 | 0 | 0 | 0 | 0 | 4 |
| DPAQRQCLSAAVWCATF | 303 | 10 | 57 | 121 | 199 | 201 | 203 | 1 | 0 | 2 | 1 | 0 | 215 |
| DPAALACLSAAVYCEMY | 304 | 6 | 49 | 132 | 200 | 199 | 246 | 0 | 1 | 2 | 0 | 1 | 203 |
| DPALQSCLSAAVYCTVS | 305 | 0 | 0 | 6 | 10 | 9 | 10 | 0 | 0 | 0 | 0 | 0 | 12 |
| DPAVQSCLSAAVYCAFR | 306 | 2 | 9 | 68 | 106 | 117 | 118 | 0 | 1 | 2 | 0 | 1 | 92 |
| DPAQMFCYSAAVYCAAF | 307 | 0 | 0 | 4 | 5 | 8 | 4 | 0 | 0 | 0 | 0 | 0 | 5 |
| DPAASQCFSAAVYCAWI | 308 | 0 | 0 | 8 | 13 | 16 | 13 | 0 | 0 | 0 | 1 | 0 | 9 |
| DPAEQRCLSAAVYCAWH | 309 | 1 | 3 | 16 | 18 | 36 | 26 | 0 | 1 | 1 | 0 | 0 | 30 |
| DPAHVRCTSAALWCNIF | 310 | 0 | 0 | 3 | 4 | 5 | 7 | 0 | 0 | 0 | 0 | 0 | 2 |
| DPAAQACLSAAVFCAWY | 311 | 5 | 19 | 45 | 86 | 88 | 97 | 0 | 0 | 0 | 0 | 0 | 98 |
| DPALRTCLSAAVFCAVA | 312 | 0 | 6 | 10 | 18 | 15 | 25 | 0 | 0 | 1 | 0 | 1 | 24 |
| DPASASCLSAAIYCSMF | 313 | 5 | 17 | 122 | 255 | 247 | 297 | 0 | 0 | 1 | 0 | 0 | 235 |
| DPAQLNCLSAAMYCALY | 314 | 2 | 19 | 49 | 95 | 104 | 111 | 0 | 0 | 1 | 0 | 0 | 87 |
| DPAQRQCLSAAVFCATL | 315 | 3 | 24 | 137 | 266 | 292 | 319 | 0 | 1 | 1 | 0 | 1 | 266 |
| DPALTNCFSAAVYCAMF | 316 | 8 | 39 | 131 | 239 | 297 | 291 | 0 | 1 | 1 | 0 | 0 | 279 |
| DPASMVCLSAAVYCAMH | 317 | 1 | 15 | 38 | 77 | 82 | 91 | 0 | 2 | 0 | 0 | 1 | 87 |
| DPAQQICLSAAVFCSIF | 318 | 0 | 0 | 4 | 10 | 11 | 7 | 0 | 0 | 0 | 0 | 0 | 6 |
| DPAMIQCLSAAVYCVHY | 319 | 1 | 3 | 8 | 13 | 21 | 13 | 0 | 0 | 0 | 0 | 0 | 13 |
| DPAQRDCFSAAVYCSFL | 320 | | 210 | 40 | 88 | 98 | 99 | 1 | 0 | 1 | 0 | 0 | 98 |
| Cluster C47 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3456s_300 |
| DPAFSQCVYAAFFCREI | 321 | 1 | 0 | 2 | 20 | 28 | 18 | 0 | 0 | 0 | 0 | 0 | 11 |
| DPAFSTCVYAAVFCQNT | 322 | 0 | 0 | 1 | 8 | 7 | 12 | 0 | 0 | 0 | 0 | 0 | 4 |
| DPAFSTCVYAAIICNDT | 323 | 0 | 0 | 7 | 29 | 46 | 36 | 0 | 0 | 0 | 0 | 0 | 12 |
| DPAFSTCIYAAVMCMDQ | 324 | 0 | 0 | 3 | 19 | 21 | 29 | 0 | 0 | 0 | 0 | 0 | 14 |
| DPAFSECVYAAIYCVHS | 325 | 0 | 0 | 0 | 7 | 9 | 16 | 0 | 0 | 0 | 0 | 2 | 5 |
| DPAFSQCVYAAVVCEVQ | 326 | 2 | 0 | 11 | 55 | 91 | 102 | 1 | 0 | 1 | 1 | 1 | 54 |
| DPAFSRCVYAAVACEDH | 327 | 0 | 1 | 3 | 31 | 57 | 56 | 0 | 0 | 2 | 0 | 0 | 27 |
| DPAFSLCVYAALFCEQQ | 328 | 0 | 0 | 8 | 20 | 36 | 43 | 0 | 0 | 0 | 0 | 0 | 14 |
| DPAFSACVYAAYMCNQD | 329 | 0 | 0 | 1 | 4 | 6 | 11 | 0 | 5 | 0 | 0 | 0 | 0 |
| DPAFSQCVYAAWHCESL | 330 | 1 | 0 | 2 | 7 | 14 | 15 | 0 | 1 | 0 | 0 | 0 | 5 |
| DPALSTCVYAAVMCQDV | 331 | 0 | 0 | 2 | 4 | 8 | 8 | 0 | 0 | 0 | 0 | 0 | 3 |
| DPAFSMCVYAAVTCVST | 332 | 0 | 0 | 0 | 4 | 6 | 13 | 0 | 0 | 0 | 0 | 0 | 2 |
| DPAFSRCVYAAFTCQTF | 333 | 0 | 0 | 1 | 10 | 21 | 30 | 1 | 0 | 0 | 0 | 0 | 8 |
| DPAFSECVYAAFSCSWI | 334 | 1 | 2 | 3 | 9 | 22 | 23 | 0 | 0 | 2 | 1 | 0 | 4 |
| DPAFFTCVYAAVTCEMT | 335 | 0 | 0 | 0 | 3 | 8 | 6 | 0 | 0 | 1 | 0 | 2 | 3 |
| DPAFYECVYAAVTCTQF | 336 | 0 | 0 | 0 | 2 | 7 | 6 | 0 | 1 | 0 | 0 | 0 | 1 |
| DPALSQCVYAAVFCQNL | 337 | 0 | 2 | 0 | 4 | 14 | 11 | 3 | 0 | 1 | 0 | 1 | 1 |
| DPAFSLCVYAAVECDHA | 338 | 0 | 0 | 0 | 1 | 9 | 3 | 0 | 0 | 0 | 0 | 0 | 2 |
| DPAFSQCIYAAFQCDMQ | 339 | 0 | 0 | 0 | 2 | 11 | 6 | 0 | 0 | 1 | 2 | 0 | 2 |
| DPAFSECVYAATMCLNH | 340 | 2 | 0 | 1 | 1 | 9 | 10 | 0 | 0 | 0 | 0 | 0 | 2 |
| Cluster C48 | | Target Concentration | | | | | | | | | | | Competitor |
| Sequence | SEQ ID NO: | 10 | 30 | 100 | 300 | 1000 | 1000 | BLANK | BLANK | BLANK | BLANK | BLANK | FP3456s_300 |
| DPAYEHCVAAAYWCYLL | 341 | 1 | 0 | 11 | 30 | 32 | 36 | 0 | 0 | 0 | 0 | 0 | 27 |
| DPAREHCDSAAIFCYIL | 342 | 0 | 0 | 1 | 14 | 13 | 14 | 0 | 0 | 1 | 0 | 0 | 8 |
| DPAREHCAAAATWCOLL | 343 | 1 | 0 | 1 | 28 | 31 | 33 | 0 | 1 | 0 | 0 | 0 | 20 |
| DPAHEHCTAAASWCYIL | 344 | 0 | 0 | 3 | 8 | 11 | 12 | 0 | 0 | 0 | 0 | 0 | 8 |
| DPAQNHCDSAAVFCYIL | 345 | 0 | 0 | 1 | 8 | 10 | 11 | 0 | 0 | 0 | 0 | 0 | 3 |
| DPAHVHCQAAAQWCVLL | 346 | 0 | 1 | 1 | 6 | 10 | 9 | 1 | 0 | 0 | 0 | 0 | 4 |
| DPAREHCDSAAAWCWVL | 347 | 0 | 0 | 1 | 4 | 11 | 6 | 0 | 0 | 4 | 0 | 2 | 6 |
| DPAITHCTAAAMWCHLL | 348 | 1 | 0 | 9 | 80 | 153 | 169 | 0 | 0 | 0 | 1 | 0 | 88 |
| DPASSHCQSAAIWCYLM | 349 | 1 | 0 | 6 | 35 | 67 | 81 | 0 | 0 | 2 | 0 | 0 | 52 |
| DPASIHCAAAALWCDIL | 350 | 0 | 0 | 1 | 8 | 25 | 15 | 0 | 0 | 0 | 0 | 0 | 10 |
| DPANEHCIAAAWWCQIL | 351 | 0 | 0 | 0 | 3 | 11 | 5 | 0 | 0 | 0 | 0 | 0 | 6 |
| DPALTHCTAAAYWCYVM | 352 | 0 | 0 | 0 | 2 | 6 | 6 | 0 | 0 | 0 | 0 | 0 | 3 |
| DPALQHCAAAASWCWLM | 353 | 1 | 2 | 4 | 20 | 56 | 52 | 2 | 0 | 2 | 0 | 1 | 16 |
| DPAHHHCQAAAAWCWVM | 354 | 2 | 0 | 4 | 19 | 44 | 65 | 0 | 0 | 0 | 1 | 1 | 22 |
| DPATQHCLAAAMWCHLI | 355 | 0 | 0 | 1 | 3 | 9 | 9 | 0 | 0 | 0 | 1 | 0 | 1 |
| DPAHEHCTSAAAWCMLL | 356 | 0 | 0 | 0 | 2 | 6 | 5 | 0 | 0 | 0 | 0 | 0 | 4 |
| DPAQQHCSAAAAWCVVL | 357 | 0 | 0 | 1 | 10 | 29 | 31 | 0 | 0 | 1 | 1 | 1 | 7 |
| DPAEYHCDAAAVWCYLM | 358 | 0 | 0 | 2 | 19 | 65 | 55 | 0 | 0 | 0 | 0 | 0 | 30 |
| DPAQVHCRSAAAWCILM | 359 | 0 | 0 | 0 | 2 | 7 | 7 | 0 | 0 | 0 | 0 | 0 | 1 |
| DPARYHCEAAAAWCELL | 360 | 0 | 0 | 0 | 8 | 33 | 24 | 1 | 0 | 0 | 0 | 0 | 12 |
| Cluster C49 | | Target Concentration | | | | | | | | | | | |
| Sequence | SEQ ID NO: | 10 | 30 | 100 | 300 | 1000 | 1000 | BLANK | BLANK | BLANK | BLANK | BLANK | |
| DPADLICQLAAANCIYL | 361 | 0 | 0 | 2 | 30 | 100 | 79 | 0 | 0 | 0 | 0 | 0 | |
| DPAQWVCHMAAMNCLYQ | 362 | 0 | 1 | 0 | 4 | 10 | 14 | 0 | 0 | 0 | 4 | 0 | |
| DPAEWLCQMAAHNCIYL | 363 | 0 | 1 | 2 | 13 | 62 | 55 | 0 | 0 | 2 | 3 | 1 | |
| DPAMYICEMAAANCLYQ | 364 | 0 | 1 | 1 | 6 | 29 | 28 | 0 | 0 | 0 | 0 | 0 | |
| DPADYICYLAANNCLYH | 365 | 3 | 1 | 4 | 19 | 117 | 106 | 1 | 1 | 0 | 0 | 1 | |
| DPAEWICRMAAMNCLYQ | 366 | 0 | 0 | 4 | 10 | 54 | 80 | 0 | 0 | 0 | 2 | 0 | |
| DPAAFVCEMAANDCLYT | 367 | 0 | 1 | 0 | 2 | 10 | 8 | 0 | 0 | 0 | 0 | 0 | |
| DPATLVCTMAANNCIYR | 368 | 0 | 0 | 0 | 1 | 13 | 10 | 0 | 0 | 0 | 0 | 0 | |
| DPARWVCELAAQNCLYR | 369 | 0 | 1 | 1 | 1 | 18 | 14 | 0 | 0 | 1 | 0 | 0 | |
| DPAEWICEMAAHNCLYM | 370 | 0 | 1 | 1 | 1 | 14 | 22 | 0 | 0 | 0 | 0 | 0 | |
| DPASIICMLAADNCLYE | 371 | 0 | 0 | 0 | 0 | 7 | 7 | 0 | 0 | 0 | 0 | 0 | |
| DPADLLCHMAAQNCLYR | 372 | 1 | 0 | 0 | 0 | 15 | 4 | 0 | 0 | 2 | 0 | 0 | |
| DPAAYLCDMAAHNCLYL | 373 | 1 | 0 | 0 | 0 | 6 | 4 | 0 | 0 | 0 | 0 | 0 | |
| DPAHILCEMAARNCLYN | 374 | 3 | 1 | 1 | 1 | 10 | 2 | 2 | 3 | 1 | 3 | 2 | |
| DPADWTCVLAAHNCLYT | 375 | 0 | 0 | 0 | 1 | 3 | 9 | 0 | 4 | 4 | 2 | 4 | |

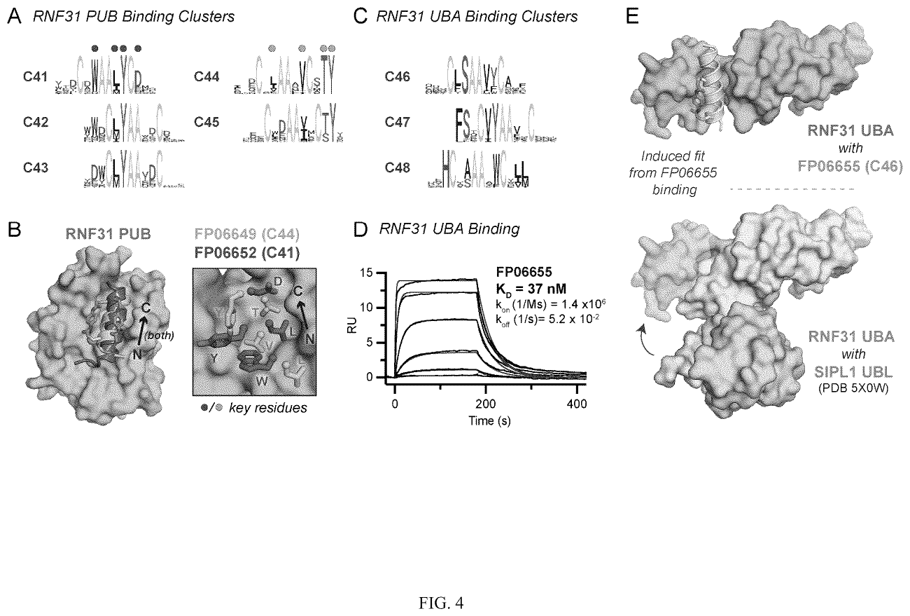

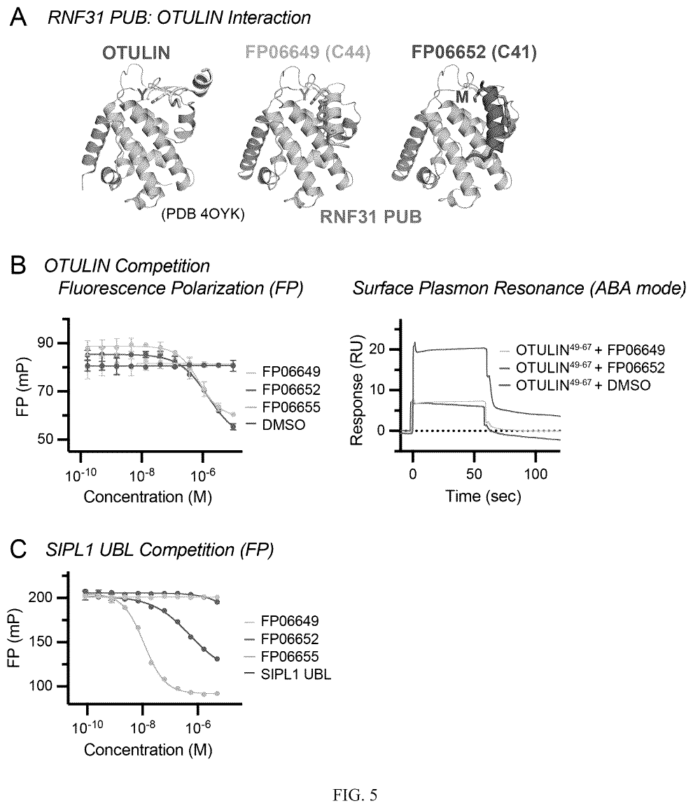

Certain data are presented in FIG. 4 and FIG. 5 as examples.

### Example 5. Stapled Peptides Binding CDK2

In some embodiments, the present disclosure provides stapled peptides that bind and modulate the function of enzymes. In some embodiments, the present disclosure provides stapled peptides that bind cyclin-dependent kinase 2, CDK2. In some embodiments, the present disclosure provides stapled peptides that modulate CDK2 activities. CDK2 is reported to be involved in regulation of the cell cycle. CDK2 is reportedly a protein target of high therapeutic interest, e.g., due to its role in cell cycle progression and its implication in CyclinE1-mediated resistance to CDK4/6-inhibitor treated cancers, but it has historically been challenging to develop CDK2-selective inhibitors, e.g., due to the close similarity of CDK2 with other CDK family members, particularly CDK1. Whereas the ATP-binding pockets of CDK2 and CDK1 are highly similar, the present disclosure encompasses the recognition that their surfaces share significantly less sequence identity so that Helicons can engage the CDK2 surface and differentiate between the two family members.

Among other things, two distinct clusters of Helicons that bound both CDK2 and CDK2 in complex with its partner CyclinE1, but not to CDK1 or CDK1 in complex with its partner CyclinA2 (C51 and C52). In some embodiments, co-crystal structures of Helicons from each of these clusters, FP19711 from Cluster C51 and FP24322 from C52, showed two allosteric binding sites with respect to the ATP pocket. A FP19711-CDK2 co-structure indicated that the N-terminal and C-terminal-most peptide residues may be not directly involved in the CDK2 interaction. In some embodiments, truncation of both the N- and C-termini to generate FP33215 improved the affinity of FP19711 to 300 nM. In some embodiments, one or more (e.g., 1, 2 or 3) or all residues to the N-terminus side of X⁴ are absent. In some embodiments, one or more (e.g., 1, 2 or 3) or all residues to the C-terminus side of X¹¹ are absent. It was also confirmed that FP19711 does not compete with ATP, and can bind to CDK2 in the presence of other ATP-competitive CDK2-binding proteins. In some embodiments, neither FP19711 nor FP33215 appeared to inhibit the kinase activity of CDK2 in a luminescence-based assay that detects ADP production. It was confirmed that FP19711 binding site can be a region on the CDK2 surface where there is high divergence from CDK1. These results highlight the utility of exploiting surface binding to achieve selectivity between closely related proteins when selectivity is difficult to achieve otherwise, e.g., with small-molecule ligands.

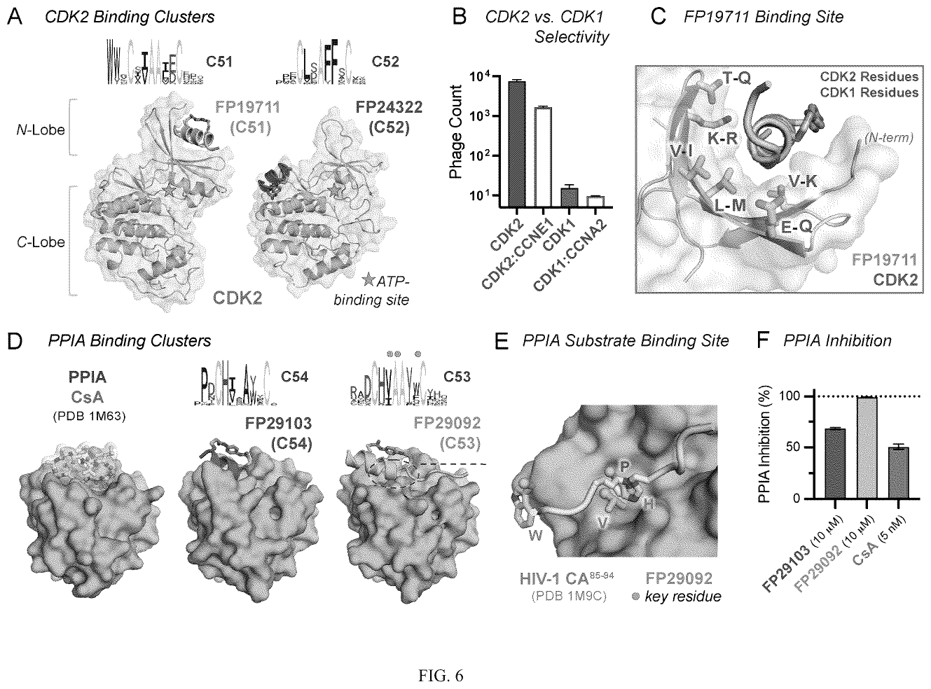

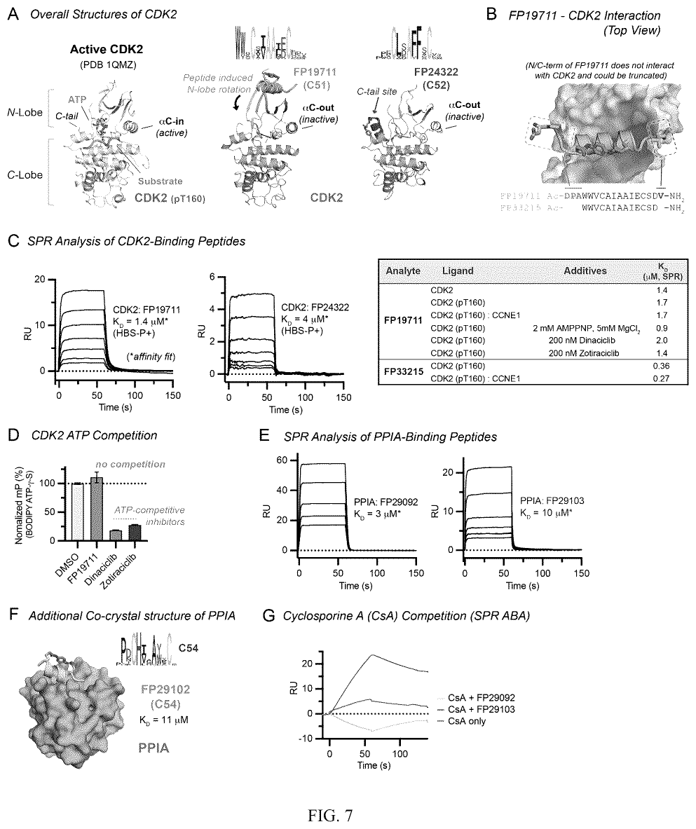

Certain results are presented in FIG. 6 and FIG. 7 as examples.

Stapled peptides of certain CDK2 binding clusters are presented below as examples:

| Cluster C51 | | Target Concentration | | | | | | | | | | | | |
|---|---|---|---|---|---|---|---|---|---|---|---|---|---|---|
| Sequence | SEQ ID NO: | 10 | 30 | 100 | 300 | 1000 | 1000 | BLANK | BLANK | BLANK | BLANK | BLANK | BLANK | BLANK |
| DPAWWVCSIAAIECESS | 376 | 0 | 0 | 10 | 85 | 65 | 77 | 0 | 0 | 0 | 0 | 0 | 0 | 0 |
| DPAWWMCTIAAIECETT | 377 | 0 | 0 | 73 | 973 | 909 | 998 | 2 | 0 | 0 | 0 | 0 | 0 | 0 |
| DPAWWRCVVAALDCVDY | 378 | 0 | 12 | 8 | 262 | 162 | 409 | 2 | 0 | 0 | 0 | 0 | 0 | 0 |
| DPAWWHCSIAALECHTT | 379 | 0 | 18 | 25 | 216 | 195 | 358 | 2 | 0 | 0 | 0 | 0 | 0 | 0 |
| DPAWWRCSVAAIDCHWN | 380 | 0 | 10 | 3 | 93 | 65 | 230 | 0 | 0 | 0 | 0 | 0 | 0 | 0 |
| DPAWWECIVAALECSDR | 381 | 0 | 0 | 0 | 62 | 76 | 90 | 0 | 0 | 0 | 0 | 0 | 0 | 0 |
| DPAWWHCVIAALDCETQ | 382 | 0 | 15 | 5 | 93 | 119 | 141 | 0 | 0 | 0 | 0 | 0 | 0 | 0 |
| DPAWYECVVAAIECHAY | 383 | 0 | 12 | 0 | 286 | 411 | 473 | 0 | 0 | 0 | 0 | 0 | 0 | 0 |
| DPAWWVCAIAAMDCHEQ | 384 | 0 | 0 | 0 | 139 | 216 | 422 | 0 | 0 | 0 | 0 | 0 | 0 | 0 |
| DPAWWSCVIAAMECQDI | 385 | 0 | 0 | 0 | 39 | 87 | 90 | 0 | 0 | 0 | 0 | 0 | 0 | 0 |
| DPAWWTCSVAAIQCWDS | 386 | 0 | 0 | 10 | 69 | 119 | 243 | 0 | 12 | 0 | 0 | 0 | 0 | 0 |
| DPAWWRCSIAAVDCEMQ | 387 | 0 | 0 | 0 | 93 | 238 | 217 | 0 | 0 | 0 | 0 | 0 | 0 | 0 |
| DPAWWRCSIAAMECHHD | 388 | 0 | 0 | 13 | 39 | 119 | 102 | 0 | 0 | 0 | 0 | 0 | 0 | 0 |
| DPAWYACIVAAVDCEHS | 389 | 0 | 0 | 8 | 46 | 206 | 115 | 0 | 0 | 0 | 0 | 0 | 0 | 0 |
| DPAWWVCAIAAIECSDV | 390 | 38 | 634 | 2330 | 12368 | 7673 | 8315 | 8 | 0 | 0 | 0 | 0 | 0 | 0 |
| DPAWWVCAVAAIECQEY | 391 | 3 | 47 | 258 | 1258 | 822 | 819 | 0 | 0 | 0 | 0 | 0 | 0 | 0 |
| DPAWWTCIIAALECEEQ | 392 | 0 | 0 | 51 | 193 | 195 | 102 | 0 | 0 | 0 | 0 | 0 | 0 | 0 |
| DPAWYECIVAAIECVEY | 393 | 0 | 0 | 79 | 131 | 54 | 115 | 0 | 0 | 0 | 0 | 0 | 0 | 0 |
| Cluster C52 | | Target Concentration | | | | | | | | | | | | |
| Sequence | SEQ ID NO: | 10 | 30 | 100 | 300 | 1000 | 1000 | BLANK | BLANK | BLANK | BLANK | BLANK | BLANK | BLANK |
| PECLEAFFDCR | 396 | 0 | 9 | 0 | 84 | 344 | 328 | 0 | 0 | 0 | 0 | 0 | 0 | 0 |
| MACVDAFFSC | 397 | 0 | 0 | 1 | 17 | 320 | 16 | 0 | 0 | 0 | 0 | 0 | 0 | 0 |
| PCLDAFFYCVT | 398 | 0 | 0 | 0 | 7 | 18 | 51 | 0 | 0 | 0 | 0 | 0 | 0 | 0 |
| PCLEAFFSCVY | 399 | 0 | 0 | 0 | 61 | 314 | 257 | 0 | 0 | 0 | 0 | 0 | 0 | 0 |
| ECLSAFFTCV | 400 | 0 | 0 | 0 | 57 | 452 | 284 | 0 | 0 | 0 | 0 | 0 | 0 | 0 |
| PMACLSAFFTC | 401 | 0 | 0 | 0 | 16 | 114 | 176 | 0 | 0 | 0 | 0 | 0 | 0 | 0 |
| FECLDAFFSC | 402 | 0 | 0 | 0 | 49 | 982 | 405 | 0 | 0 | 0 | 0 | 0 | 0 | 0 |
| CLDAFFACER | 403 | 0 | 0 | 0 | 4 | 7 | 408 | 0 | 0 | 0 | 0 | 0 | 0 | 0 |
| PECLVAFFSCV | 404 | 0 | 1 | 9 | 9 | 422 | 142 | 0 | 0 | 0 | 0 | 0 | 0 | 0 |
| PSCLSAFFSCY | 405 | 0 | 1 | 0 | 14 | 722 | 485 | 0 | 0 | 0 | 0 | 0 | 0 | 0 |
| PCMDAFFSCVW | 406 | 0 | 0 | 1 | 0 | 39 | 8 | 0 | 0 | 0 | 0 | 0 | 0 | 0 |
| PFECLSAFFSC | 407 | 0 | 0 | 0 | 1 | 212 | 44 | 0 | 0 | 0 | 0 | 0 | 0 | 0 |
| PTCLNAFFACE | 408 | 0 | 0 | 0 | 1 | 109 | 35 | 0 | 0 | 0 | 0 | 0 | 0 | 0 |
| YECLEAFFNC | 409 | 0 | 0 | 0 | 2 | 208 | 193 | 0 | 0 | 0 | 0 | 0 | 0 | 0 |
| PACVSAFFACL | 410 | 0 | 0 | 0 | 0 | 151 | 19 | 0 | 0 | 0 | 0 | 0 | 0 | 0 |
| CLSAFFMCSS | 411 | 0 | 0 | 0 | 0 | 90 | 50 | 0 | 0 | 0 | 0 | 0 | 0 | 0 |
| WSCLSAYFSC | 412 | 0 | 0 | 0 | 0 | 19 | 189 | 0 | 0 | 0 | 0 | 0 | 0 | 0 |
| CLSAFFSCTY | 413 | 29 | 0 | 0 | 0 | 111 | 330 | 0 | 0 | 0 | 0 | 0 | 0 | 0 |

### Example 6. Stapled Peptides Binding PPIA

In some embodiments, the present disclosure provides stapled peptides that bind peptidyl-prolyl cis-trans isomerase cyclophilin A, PPIA. In some embodiments, the present disclosure provides stapled peptides that modulate PPIA activities. PPIA is reported to bind non-alpha-helical peptide substrates and catalyzes the cis-trans isomerization of proline residues.

In some embodiments, peptidyl-prolyl cis-trans isomerases (PPIases) are reported to be a family of enzymes that recognize a diverse range of proline-containing polypeptide substrates, and are targets of the “trimerizer” natural products CsA and Sanglifehrin A that bind PPIA to form ternary complexes with the proteins Calcineurin and IMPDH2, respectively.

In some embodiments, the present disclosure provides a pair of shifted clusters that competed with CsA both in the phage screen and in in vitro biochemical assays. Co-crystal structures of Helicons from both Clusters C53 and C54 confirmed that they can bind the site ordinarily occupied by peptide substrates or natural products, providing another instance where provided technologies were able to identify an alpha-helical binding solution to a site ordinarily recognized by peptides in a non-alpha-helical conformation. A PPIase assay showed that these Helicons inhibit PPIA activity, as expected given their orthosteric binding mode, demonstrating the ability of Helicons to directly block enzymatic function.

Certain results are presented in FIG. 6 and FIG. 7 as examples.

Stapled peptides of certain PPIA binding clusters are presented below as examples:

| Cluster C53 | | Target Concentration | | | | | | | | | | Competitor |
|---|---|---|---|---|---|---|---|---|---|---|---|---|
| Sequence | SEQ ID NO: | 10 | 30 | 100 | 300 | 1000 | 1000 | BLANK | BLANK | BLANK | BLANK | Cyclosporin |
| DPAYYDCHIAAYFCRTQ | 414 | 16 | 62 | 77 | 106 | 93 | 131 | 0 | 0 | 0 | 0 | 0 |
| DPARADCHVAAYWCYHR | 415 | 0 | 1 | 6 | 6 | 7 | 6 | 0 | 0 | 0 | 0 | 0 |
| DPASYDCHVAAYFCNFA | 416 | 6 | 10 | 176 | 197 | 190 | 227 | 0 | 0 | 0 | 0 | 0 |
| DPAAFSCHVAAWQCHTL | 417 | 0 | 1 | 156 | 199 | 142 | 190 | 0 | 0 | 0 | 0 | 0 |
| DPALFDCHIAAYQCIFY | 418 | 0 | 0 | 25 | 29 | 20 | 55 | 0 | 0 | 0 | 0 | 0 |
| DPARSDCHVAAYWCYHQ | 419 | 0 | 0 | 4 | 5 | 6 | 6 | 0 | 0 | 0 | 0 | 0 |
| DPATMNCHVAAYFCIVE | 420 | 0 | 0 | 10 | 13 | 10 | 19 | 0 | 0 | 0 | 1 | 0 |
| DPARADCQVAAYWCYHQ | 421 | 0 | 3 | 13 | 16 | 20 | 21 | 0 | 0 | 0 | 0 | 0 |
| DPARTDCHVAAYWCYHQ | 422 | 0 | 5 | 13 | 14 | 18 | 25 | 0 | 0 | 0 | 0 | 0 |
| DPAQLNCHVAAFFCFQS | 423 | 0 | 0 | 21 | 27 | 125 | 8 | 0 | 0 | 0 | 0 | 0 |
| DPARADCHVAAYWCYQQ | 424 | 0 | 1 | 6 | 8 | 10 | 10 | 0 | 0 | 0 | 0 | 0 |
| DPARADCRVAAYWCYHQ | 425 | 0 | 1 | 6 | 6 | 11 | 7 | 0 | 0 | 0 | 0 | 0 |
| DPAAHDCHIAAYWCLNY | 426 | 0 | 7 | 116 | 208 | 201 | 121 | 0 | 0 | 0 | 0 | 0 |
| DPAQHDCHVAAYQCIWE | 427 | 0 | 52 | 273 | 416 | 616 | 456 | 0 | 0 | 0 | 0 | 0 |
| DPARADCHVAAYWCHHQ | 428 | 0 | 0 | 4 | 5 | 8 | 7 | 0 | 0 | 0 | 0 | 0 |
| DPARYSCHVAAYECILN | 429 | 0 | 0 | 17 | 14 | 41 | 27 | 0 | 0 | 0 | 0 | 0 |
| DPARADCHVAAYWCYHE | 430 | 0 | 1 | 4 | 7 | 9 | 7 | 0 | 0 | 0 | 0 | 0 |
| DPAQADCHIAAYLCLFD | 431 | 0 | 0 | 10 | 19 | 25 | 18 | 0 | 0 | 0 | 0 | 0 |
| DPARAACHVAAYWCYHQ | 432 | 0 | 1 | 3 | 7 | 6 | 7 | 0 | 0 | 0 | 0 | 0 |

| Cluster C54 | | Target Concentration | | | | | | | | |
|---|---|---|---|---|---|---|---|---|---|---|
| Sequence | SEQ ID NO: | 10 | 30 | 100 | 300 | 1000 | 1000 | BLANK | BLANK | BLANK |
| PNCHVMAYWCW | 434 | 0 | 0 | 1 | 8 | 10 | 7 | 0 | 0 | 0 |
| PNCHVEAYFCM | 435 | 0 | 1 | 8 | 68 | 98 | 63 | 0 | 0 | 0 |
| PHDCHVNAWYC | 436 | 0 | 0 | 0 | 26 | 21 | 45 | 0 | 0 | 0 |
| PDCHVAAYICY | 437 | 0 | 0 | 3 | 38 | 54 | 44 | 0 | 0 | 0 |
| PDCHLNAYWCM | 438 | 0 | 0 | 1 | 47 | 47 | 115 | 0 | 0 | 0 |
| PTCHIDAWVCY | 439 | 0 | 0 | 0 | 12 | 18 | 26 | 0 | 0 | 0 |
| PFCHIEAYYCW | 440 | 0 | 0 | 0 | 14 | 46 | 22 | 0 | 0 | 0 |
| PNCHLYAWECY | 441 | 0 | 0 | 0 | 4 | 5 | 19 | 0 | 0 | 0 |
| PDCHIYAFYCL | 442 | 0 | 0 | 1 | 5 | 8 | 18 | 0 | 0 | 0 |
| PDCHVAAWMCF | 443 | 0 | 0 | 0 | 8 | 16 | 26 | 0 | 0 | 0 |
| PTCHIHAYHCY | 444 | 0 | 0 | 3 | 5 | 31 | 8 | 0 | 0 | 0 |
| PDCHIASWFCS | 445 | 0 | 0 | 0 | 3 | 25 | 5 | 0 | 0 | 0 |
| PDCHIQAYICH | 446 | 0 | 0 | 0 | 4 | 6 | 35 | 0 | 0 | 0 |
| PDCHIRAYVCH | 447 | 0 | 0 | 1 | 8 | 11 | 120 | 0 | 0 | 0 |
| PNCHVEAWYCI | 448 | 0 | 0 | 0 | 1 | 12 | 12 | 0 | 0 | 0 |
| PDCHIRAYMCN | 449 | 0 | 0 | 0 | 0 | 20 | 26 | 0 | 0 | 0 |
| PDCHIAAWYCR | 450 | 0 | 0 | 0 | 0 | 11 | 10 | 0 | 0 | 0 |
| PECHVEAYFCE | 451 | 0 | 0 | 0 | 0 | 24 | 22 | 0 | 0 | 0 |
| PTCHVQAWYCW | 452 | 0 | 0 | 0 | 0 | 10 | 29 | 0 | 0 | 0 |
| PYDCHIAAWMC | 453 | 0 | 0 | 0 | 0 | 20 | 35 | 0 | 0 | 0 |

### Example 7. Stapled Peptides Binding PD-L1

In some embodiments, the present disclosure provides stapled peptides that bind PD-L1. In some embodiments, the present disclosure provides stapled peptides that modulate PD-L1 activities. In some embodiments, the present disclosure provides stapled peptides that bind extracellular domains or (ECDs). In some embodiments, the present disclosure provides stapled peptides that bind extracellular domains or (ECDs) of PD-L1.

It has been reported that ECD of the transmembrane protein Programmed cell death 1 ligand 1 (PD-L1) can bind to the ECD of Programmed cell death protein 1 (PD-1) to suppress T-cell function. To the Applicant's knowledge, the PD-L1 ECD has not been shown to bind a-helices. In some embodiments, the present disclosure provides two clusters of Helicons (C61 and C62) that bind PD-L1. In some embodiments, they compete with PD-1. In some embodiments, on-phage competition was confirmed. Among other things, in vitro competition enzyme-linked immunoassay (ELISA) confirmed that FP28132 from Cluster C61 compete with PD-L1. FP30790 from Cluster C62 did not show competition in the utilized ELISA format, but competition SPR confirmed that PD1 blocks its binding to PD-L1. Co-crystal structures of PD-L1 with these two Helicons showed that the two clusters both engage the PD-1 binding surface of PD-L1, but occupy distinct alpha-helical sites on it.

It was observed that an asymmetric unit of the FP28132/PD-L1 co-crystal structure contained a symmetric dimer of two FP28132/PD-L1 complexes. Two other Helicons, FP28135 and FP28136 derived from the same PD-L1-binding phage cluster, were also structurally characterized and a similar assembly was observed. An extensive series of contacts between both the two Helicon protomers and the two PD-L1 protomers were shown. To assess whether this is an indication that the FP28132/PD-L1 complex is a dimer in solution, analytical size-exclusion chromatography (SEC) and time-resolved fluorescence energy transfer (TR-FRET) experiments were performed with PD-L1 in the presence and absence of FP28132, FP30790, a non-PD-L1 binding mutant of FP28132 that differs by one residue (FP28141), and BMS-1001, a small-molecule PD-L1 binder that has also been shown to induce protein dimerization. Evidence of PD-L1 dimerization in the presence of both BMS-1001 and FP28132, but not in the presence of FP30790 or FP28141, was observed. Among other things, these findings demonstrate that the provided technologies can provide multiple a-helix binding sites to a relatively small ECD that was not previously known to have any, as well as Helicons that can induce protein dimerization.

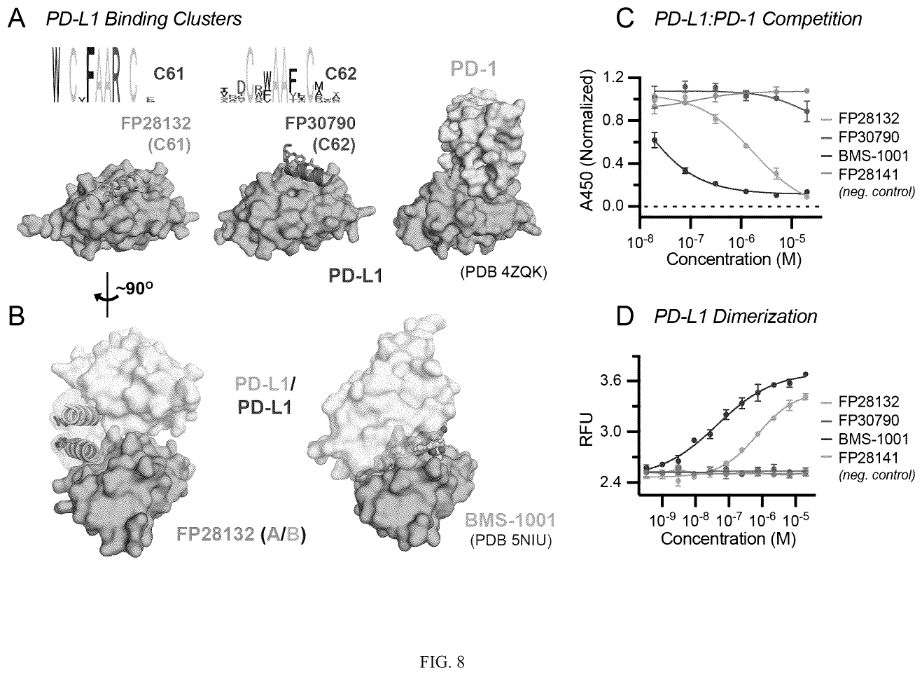

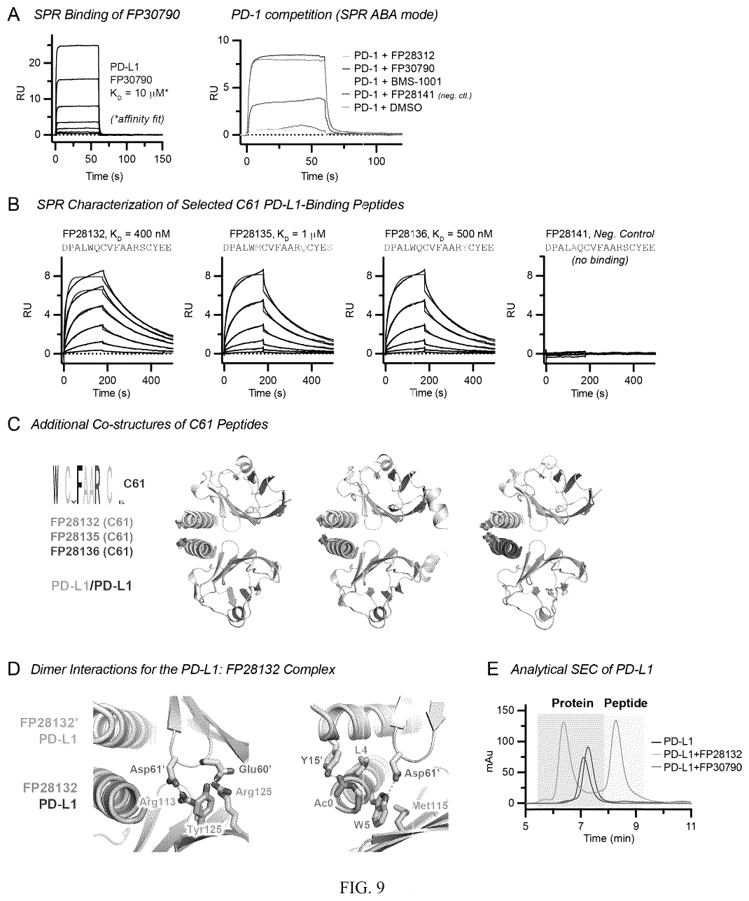

Certain results are presented in FIG. 8 and FIG. 9 as examples.

Stapled peptides of certain PD-L1 binding clusters are presented below as examples:

| Cluster C61 | | Target Concentration | | | | | | | | | | Competitor |
|---|---|---|---|---|---|---|---|---|---|---|---|---|
| Sequence | SEQ ID NO: | 10 | 30 | 100 | 300 | 1000 | 1000 | BLANK | BLANK | BLANK | BLANK | PD1_300 |
| DPALWQCVFAARSCYEE | 454 | 667 | 1034 | 1256 | 1411 | 599 | 730 | 6 | 0 | 0 | 0 | 496 |
| DPAVWACTFAARYCHEA | 455 | 0 | 26 | 269 | 259 | 105 | 91 | 0 | 0 | 0 | 0 | 0 |
| DPATWRCVFAARACSMQ | 456 | 0 | 0 | 0 | 121 | 7 | 75 | 0 | 0 | 0 | 0 | 0 |
| DPALWMCVFAARQCYES | 457 | 97 | 3 | 3 | 77 | 7 | 83 | 1 | 0 | 0 | 0 | 4 |
| Cluster C62 | | Target Concentration | | | | | | | | | | Competitor |
| Sequence | SEQ ID NO: | 10 | 30 | 100 | 300 | 1000 | 1000 | BLANK | BLANK | BLANK | BLANK | PD1_300 |
| DPAAADCQWAAFLCRVY | 458 | 0 | 0 | 12 | 47 | 60 | 62 | 0 | 0 | 0 | 0 | 0 |
| DPAYQTCWWAAYICAQY | 459 | 0 | 0 | 0 | 5 | 6 | 5 | 0 | 0 | 0 | 0 | 0 |
| DPAVDDCWVAATICRIM | 460 | 0 | 0 | 0 | 13 | 12 | 31 | 0 | 0 | 0 | 0 | 0 |
| DPASEDCRWAAFLCAHY | 461 | 0 | 0 | 9 | 110 | 191 | 289 | 0 | 0 | 0 | 0 | 0 |
| DPAIEECRFAAFQCMYA | 462 | 0 | 0 | 0 | 2 | 7 | 5 | 0 | 0 | 0 | 0 | 0 |
| DPAIADCRFAAFRCMYA | 463 | 0 | 0 | 0 | 2 | 12 | 35 | 0 | 0 | 0 | 0 | 0 |
| DPAVQSCWWAAYTCAIQ | 464 | 0 | 0 | 0 | 2 | 23 | 38 | 0 | 0 | 0 | 0 | 0 |
| DPASSDCSWAAFRCMFS | 465 | 0 | 0 | 0 | 1 | 15 | 14 | 0 | 0 | 0 | 0 | 0 |
| DPAVDYCRFAAFSCQWA | 466 | 0 | 12 | 0 | 0 | 13 | 19 | 0 | 0 | 0 | 0 | 0 |
| DPAIRDCAWAAFLCMHE | 467 | 0 | 0 | 0 | 5 | 18 | 17 | 0 | 3 | 0 | 2 | 0 |

### Example 8. Trimerizers

In some embodiments, the present disclosure provides technologies for designing, identifying, characterizing, producing and using stapled peptides that can bridge more than one target of interest. In some embodiments, the present disclosure provides trimerizers that can bridge targets of interest. Certain useful technologies are presented below as examples.

Naive Phage Library: In some embodiments, collections of stapled peptides, etc. phage-displayed peptide libraries are constructed using the filamentous bacteriophage vector M13KE (New England Biolabs, Ipswich, MA). In some embodiments, protocol guidelines in the New England Biolabs Ph.D.™ Peptide Display Cloning System kit were utilized. Briefly, library oligonucleotides are chemically synthesized using a mix of trimer phosphoramides (Glen Research, Sterling, VA) without codons for cysteine, lysine, proline, and glycine, annealed, extended, and ligated into a digested M13KE vector. KpnI and EagI sites are used to digest M13KE for 5 h at 37° C. The insert coding strand contains the library sequence, 5′-CATGCCCGGGTACCTTTCTATTCTCACTCTGCGGATCCGGCGXXXTGCXXGCAGCAXXTGTXXXGGTGGTTCTGGCTGGGGTCGTGGTTC-3′ (SEQ ID NO: 21), where X represents a single trimer phosphoramide incorporation, flanked by KpnI site. The antisense strand complements the 3′-end of the insert coding strand to allow Klenow extension, 5′-CATGTTTCGGCCGAACCACGACCTGCGCCAGAACCAC-3′ (SEQ ID NO: 22). The antisense strand possesses an EagI site. Annealed library inserts are digested with EagI and KpnI for 5 h at 37° C. The digested products are purified using Monarch PCR and DNA cleanup kit (New England Biolabs, Ipswich, MA), followed by T4 ligation (New England Biolabs, Ipswich, MA). The resulted library-containing vector is transformed into E. coli strain ER2738 (Lucigen, Middleton, WI) by electroporation. Post-rescue culture is used to determine library diversity. Phage particles containing library members are amplified by adding the post-rescue electroporated cells to a 500 mL robustly growing at 37° C. E. coli culture at early-lag phase (OD600=0.01). Phage propagation continues for 5 h in LB media supplemented with 100 uM of MgCl₂ and CaCl₂, with shaking at 37° C. E. coli cells are pelleted at 5000×g and the phage particles are precipitated from the supernatant by addition of 1/5 volume of 20% (w/v) polyethylene glycol 8000, 2.5 M NaCl, followed by overnight incubation at 4° C., pelleted the second time at 5000×g, and resuspended in Tris-buffered saline (TBS). Phage-displayed peptide libraries are further purified by repeating the precipitation, pelleting, and resuspension steps. Phage-displayed peptide libraries are covalently crosslinked (stapled) by diluting the phage particle solution in 1×TBS to an OD₆₀₀ of 1.0 and adding dithiothreitol to a concentration of 1 mM, followed by dialysis against 100 volumes of 20 mM NH₄CO₃, 2 mM EDTA, pH -8 for 30-60 min, followed by addition of the dialyzed phage to a solution of crosslinker prepared in 20 mM NH₄CO₃, 2 mM EDTA, pH -8 (final crosslinker concentration is 200 uM, note that crosslinker may not dissolve completely, in some embodiments, brief sonication immediately prior to mixing with phage is performed to disperse the solid into a fine suspension) and incubation with rotation for 2 h at 32° C. Excess crosslinker is removed first by pelleting at 5000×g and decanting, followed by addition of dithiothreitol to a concentration of 0.25 mM with incubation for 10 min, and then addition of iodoacetamide to a concentration of 0.75 mM with incubation for a further 10 min. Ellman's reagent (5,5′-dithiobis-(2-nitrobenzoic acid) can be used to track the quenching of DTT until all thiols are capped. Phage particles are further purified by repeating the precipitation, pelleting, and resuspension steps described above, then are stored as solutions in 50% v/v glycerol in TBS at −80° C. at >10¹² pfu/mL. Finally, individual phage library members are characterized by DNA sequencing. Well-separated blue plaques are picked from the LB/IPTG/Xgal Agar plates in 50 μL of water. 2 μL of a resuspended template is mixed with 23 μL of the amplification master mix containing OneTaq DNA polymers (NEB, Ipswitch, MA) and two 10 uM M13KE sequence-specific amplification primers (NEB, Ipswitch, MA). Routine PCR is performed, and samples are submitted for standard Sanger sequencing (GENEWIZ, Cambridge, MA). Prior to library screening, deep sequencing (as described below) of the library is preformed to ensure that it is high in sequence diversity.

Trimerizer Library: Following identification of peptide clusters specific for a first target of interest, e.g., a presenter protein of interest, based on a naive presenter screen, trimerizer library oligonucleotides are designed. First target of interest, e.g., presenter, specific clusters of various sizes can be used, ranging in size from 10-mer to 20-mer. As an example of a Trimerizer library design, a design based on a presenter specific 20-mer cluster, X₁X₂X₃X₄W₅E₆C₇X₈E₉A₁₀A₁₁(F/I/L/M)₁₂X₁₃C₁₄X₁₅(F/Y)₁₆(F/Y)₁₇X₁₈X₁₉X₂₀ (SEQ ID NO: 23), is used. Briefly, codons of conserved or semi-conserved residues (enriched amino acid residues for a first target of interest) responsible for binding with a first target of interest, e.g., a presenter protein, are fixed or partially randomized to bias the library for retained affinity towards a first target of interest, e.g., a chosen presenter protein. In some embodiments, for partial randomization, semi-degenerate codons are used to randomize to sample a fixed subset of residues conserved within that position reducing the total sequence space required for full randomization at every position. For example, in position 12, which has four potential residues (F/L/I/M), semi-degenerate codon, WTK, is used where W represents A or T and K represents G or T, coding for phenylalanine (TTT), leucine (TTG), methionine (ATG), and isoleucine (ATT), thus representing all residues observed within a identified cluster specific for a first target of interest, e.g. a presenter. For position 16 and 17 within the example cluster, semi-degenerate codons, TWT, are used coding for tyrosine (TAT) and phenylalanine (TTT) in a similar manner presenting all residues observed within an identified cluster specific for a first target of interest, e.g., a presenter. All other residues within the displayed peptide with no apparent preference for binding to a first target of interest, e.g., a presenter, (represented as Xs within the cluster) are randomized to any other amino acid, e.g., except cysteine, lysine, proline and glycine. Trimerizer libraries are built using multiple oligonucleotides from various designs based on specific binding sequences for a first target of interest, e.g., a presenter. Library oligonucleotides can be chemically synthesized using a mix of trimer phosphoramides (Glen Research, Sterling, VA) lacking codons for amino acids that are not to be included, e.g., cysteine, lysine, proline, and glycine, annealed, extended, and ligated into a digested with KpnI and EagI restriction enzymes M13KE vector. Example oligonucleotide insert coding strand containing the Trimerizer library sequence is shown below. 5′-CATGCCCGGGTACCTTTCTATTCTCACTCTGCGCCGXXXXTGGGAATGTXGAAGCAGCAWTKXTGTXTWTTWTXXXGGTGGTTCTGGCGCAGGTCGTGGTTC-3′ (SEQ ID NO: 24), where X represents a single trimer phosphoramide incorporation, flanked by KpnI site. The antisense strand complements the 3′ end of the sense strand to allow Klenow extension, 5′-CATGTTTCGGCCGAACCACGACCTGCGCCAGAACCAC-3′ (SEQ ID NO: 22). The antisense strand possesses an EagI site. Trimerizer Library construction protocol can follow the previously described Naive Phage Library construction protocol.

Naive Library Presenter Screening: In some embodiments, phage screening is performed using biotinylated proteins bound to streptavidin magnetic beads (Dynabeads MyOne Streptavidin T1, ThermoFisher Scientific, Waltham, MA). About 10¹⁰ phage particles are added to each phage screening sample, to ensure approximately 100 copies of each of the 10′ library members. Peptide-displayed phage libraries are incubated with streptavidin magnetic beads for 1 h at room temperature in a buffer of 1×TBS, 1 mM MgCl2, 1% (w/v) BSA, 0.1% Tween-20, 0.02% (w/v) sodium azide, 5% (w/v) nonfat milk to remove bead-binding library members. For each screening condition, 100 μL of 2 uM biotinylated desired presenter protein is captured with 0.5 mg of streptavidin-coated magnetic beads that have been previously blocked with 1% BSA, 0.1% Tween-20, 2% glycerol in 1×TBS pH 7.4 at room temperature for 15 min in 96-well plates, then the supernatant is removed using a plate magnet and the beads are promptly but gently resuspended in 50 μL of the same buffer. 150 μL of the depleted phage library is added to each well for 200 uL final volume, plates are sealed, and the screening reactions are incubated at room temperature for 45 min, with rotation to maintain beads in solution. Inspection of these solutions is performed to confirm that beads have not aggregated or crashed out of solution, which can be indicative of protein aggregation. Biochemical tests should be performed to ensure that target proteins are stable under the conditions of the screen, and conditions should be adjusted accordingly if required. Following binding, beads are washed 5 times with ice-cold 1×TBS, 1 mM MgCl2, 1% (w/v) BSA, 0.1% Tween-20, 0.02% (w/v) sodium azide, 2% (w/v) glycerol. This can be performed with an automated bead handler such as a KingFisher (Thermo Fisher) or manually. Target-bound phage library members are then directly processed for NGS.

Trimerizer Phage Screening: In some embodiments, trimerizer phage screening is performed using the similar procedure described above. In some embodiments, a key difference is that Trimerizer library is incubated with a first target of interest, e.g., a presenter protein, after removal of the bead-binding phage library members prior to the incubation with biotinylated proteins bound to streptavidin magnetic beads (Dynabeads MyOne Streptavidin T1, ThermoFisher Scientific, Waltham, MA). To identify first target of interest (e.g., presenter)-dependent peptide members, in some embodiments, a target protein (e.g., a second target of interest) is screened with depleted peptide library in the presence and absence of a first target of interest, e.g. a presenter protein. In some embodiments, prior to addition to captured protein, depleted peptide-displayed phage library is split into two portions, and a first target of interest, e.g., a presenter protein is added to one portion to a final concentration of 10 uM. 150 μL of the depleted peptide-displayed phage library without presenter protein is then added to replicate highest concentration of a target protein and blank wells for 200 uL final volume. To remaining wells, 150 μL of the depleted peptide-displayed phage library mixed with 10 uM of a first target of interest, e.g., a presenter protein, is added to 50 μL of a target of interest, plates are sealed, and the screening reactions are incubated at room temperature for 45 min, with rotation to maintain beads in solution. The rest of the experiment is performed similar to the above described phage screening with the exception of the addition of a first target of interest at a suitable concentration, e.g., a presenter protein at a final concentration of 10 uM to the wash buffer (ice-cold 1×TBS, 1 mM MgCl2, 1% (w/v) BSA, 0.1% Tween-20, 0.02% (w/v) sodium azide, 2% (w/v) glycerol).

Next-Generation Sequencing: To sequence the phage-displayed peptide library members, phage particles are denatured from beads at 95° C. for 15 min in 25 mM Tris pH 8, 50 mM NaCl, 0.5% Tween-20. Prior to boiling, 10,000 copies of a phage clone of known sequence (not a library member) are spiked in to each well to enable cross-well normalization of sequence reads. A two-step low-cycled PCR is performed to introduce Illumina adaptors and 10 bp TruSeq DNA UD Indexes (Illumina, San Diego, CA) to the 3′ and 5′ ends of amplicons with M13KE Forward and M13KE Reverse primers (M13KE Forward: 5′-TCGTCGGCAGCGTCAGATGTGTATAAGAGACAGTTCGCAATTCCTTTAGTGG-3′ (SEQ ID NO: 25) and M13KE Reverse: 5′-GTCTCGTGGGCTCGGAGATGTGTATAAGAGACAGATTTTCTGTATGGGATTTTGCTAA-3′ (SEQ ID NO: 26)) similar to Illumina's 16S Metagenomic Sequencing Library Preparation protocol. The NGS library is sequenced by an Illumina NovaSeq platform using a 2×150 bp high-output kit (Illumina, San Diego, CA).

Hit ID and Clustering: NGS reads are trimmed for quality (Phred score >18) and filtered for sequences that matched the design of the phage library. Counts for each unique sequence are tallied, and then normalized by the counts of the spike-in sequence added to each sample. A metric called Hit Strength is computed for each sequence as the fold change between the normalized counts in the highest target concentration sample with presenter and the normalized counts in the target (no presenter) samples (averaged across experimental replicates). By using target wells with no first target of interest, e.g., presenter as “blanks, presenter-dependent binding is observed. This approach eliminates sequences that show binding to target (e.g., a second target of interest) alone or binding to a free first target of interest, e.g., presenter, alone. 0 counts are observed for a sequence in target only blank samples, a count of 0.5 is used to prevent dividing by zero.

Sequences with a hit strength greater than 5 are subjected to hierarchical clustering to identify sequence families. Pairwise distances between sequences i and j are computed using

$${distance}_{ij} = ( 1 - \frac{{score}_{ij}}{{score}_{ii}} ) ( 1 - \frac{{score}_{ij}}{{score}_{jj}} ) ,$$

where scoreᵢⱼ is the alignment scores based on a modified BLOSUM62 substitution matrix. In some embodiments, tryptophan-tryptophan match score was decreased from 11 to 7 in the BLOSUM62 substitution matrix to prevent overly-biasing clustering towards tryptophans. Hierarchical clustering using average linkage is used to group the sequences into families. To avoid clustering using a large number of sequences, which is computationally intensive and can make it difficult to identify small clusters, in some embodiments, multiple rounds of clustering were performed. First, sequences were sort by descending hit strength. The top 1000 (first round of clustering), top 2000 (2^{nd} round of clustering), or top 3000 (≥3^{rd} round of clustering) sequences, were then taken, and was subjected to clustering as described above. Clusters of sequences with high sequence similarity (sequence “families”) are identified at each round and removed from the pool of sequence for subsequent rounds. Sequences subjected to 3 rounds of clustering without falling into a sequence family are similarly dropped from subsequent rounds of clustering under the assumption that they do not belong to a sequence family. The process is halted after 10 rounds of clustering, or when no sequences remained in the list.

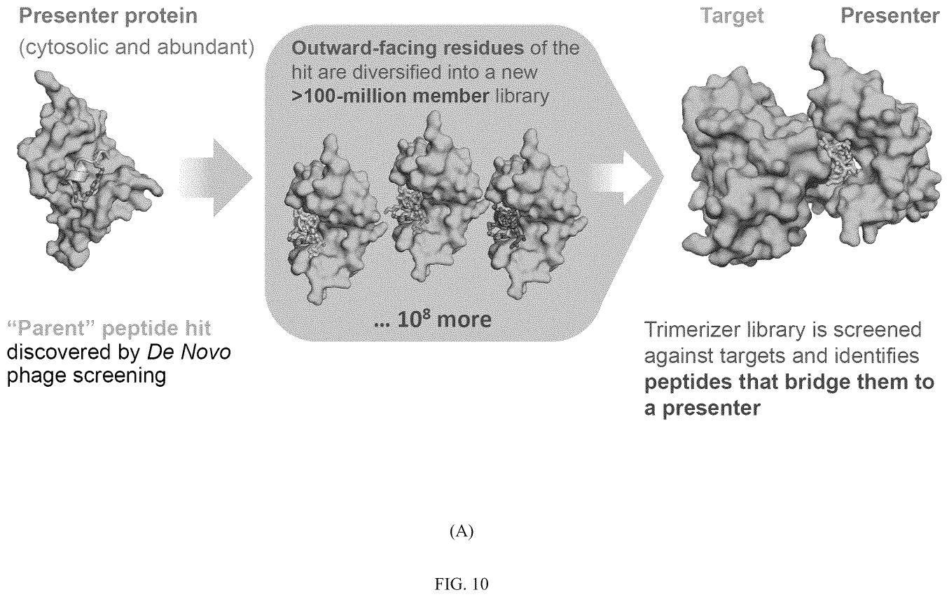

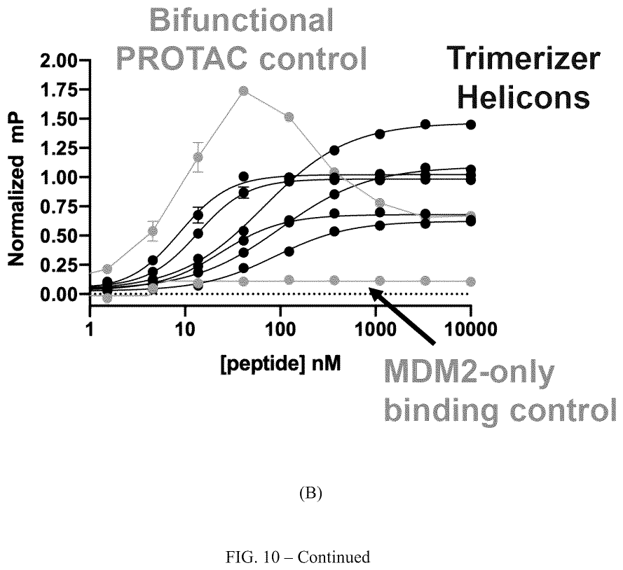

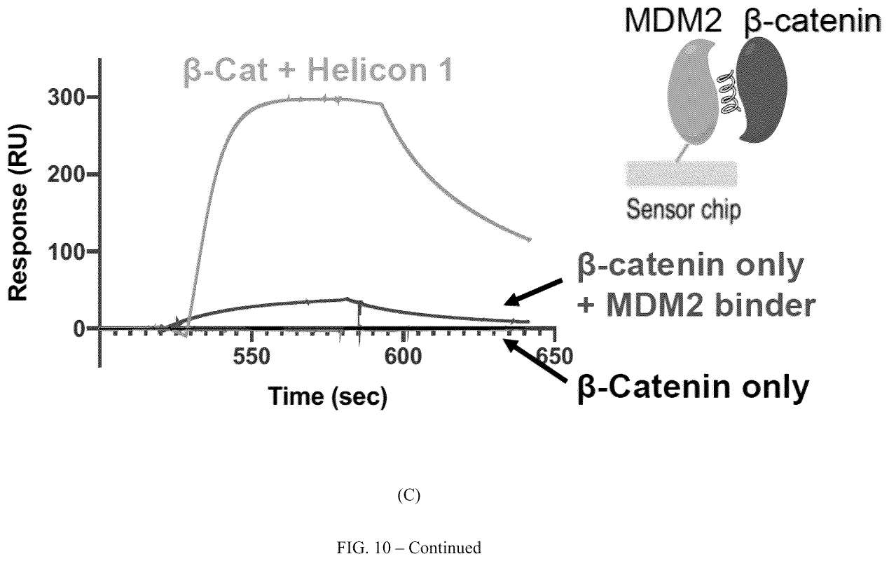

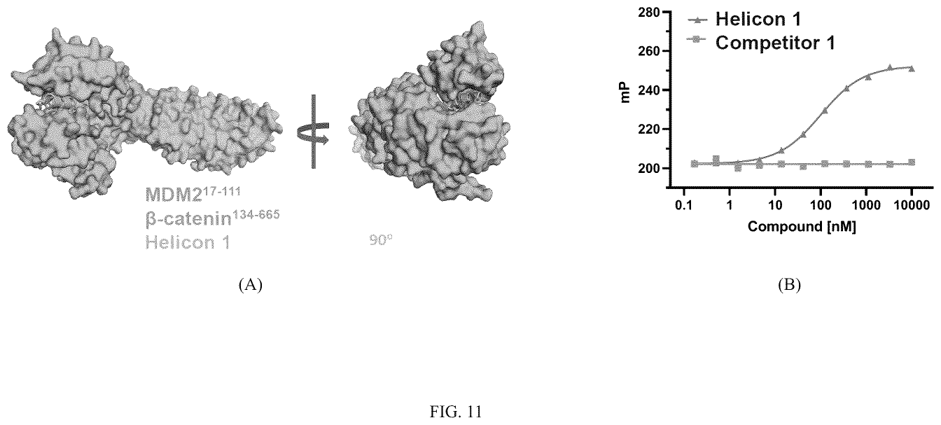

Certain useful technologies and data are described in FIG. 10 and FIG. 11 as examples. In some embodiments, trimerizers are biochemically validated. In some embodiments, trimerizers provide different profiles compared to other binders, e.g., PROTAC. In some embodiments, a hook effect was observed for a given PROTAC control. In some embodiments, no hook effect is observed with trimerizers. In some embodiments, trimerizers provide cooperative binding. In some embodiments, the present disclosure provides trimerizers for beta-catenin and MDM2. In some embodiments, a trimerizer is Ac-Pro-Met-Glu-Gln-Gln-Ala-Ile-Cys-Phe-Gln-Ala-Ala-Trp-Met-Cys-Leu-Ala-Asp-Asp-Trp-Thr-NH2, wherein the two Cys are stapled (SEQ ID NO: 27). In some embodiments, a trimerizer is Ac-Pro-Trp-Lys-Tyr-Glu-Gln-Val-Cys-Tyr-Gln-Ala-Ala-Trp-Gln-Cys-Leu-Ser-Asp-Asp-Trp-Asp-NH2, wherein the two Cys are stapled (SEQ ID NO: 28).

### Example 9. Griptides

In some embodiments, the present disclosure provides technologies for designing, identifying, characterizing, producing and using stapled peptides, e.g., those comprising two stapled peptide moieties or alpha-helical structures linked together. In some embodiments, such griptides can provide high affinity. In some embodiments, hydrophobic interface residues support dimer formation. In some embodiments, surface-exposed residues are diversified into a library as described herein, e.g., with about 10′ members or more. Certain useful technologies are presented below as examples.

Griptide Phage Library construction: In some embodiments, collections of griptides, e.g., phage-displayed Griptide libraries are constructed using a modified phagemid vector according to manufacturer's protocol (Antibody Design Labs, San Diego, CA). pADL-100 phagemid vector (Antibody Design Labs, San Diego, CA), is modified to include a FLAG-tag (DYKDDDDK (SEQ ID NO: 29)) sequence, two additional restriction sites (KpnI and Sac7), a placeholder sequence, and an extended linker sequence between the 3′-end of the leader sequence and 5′-end of the minor coat protein, pIII, by utilizing available restriction cutting sites NocI and SpeI. Griptide DNA-library oligonucleotides are chemically synthesized using trimer phosphoramides technology (Glen Research, Sterling, VA) without codons for cysteine, annealed, extended, and ligated into a digested modified pADL-100 phagemid vector. The antisense strand contains the library sequence, 5′-GGGCCGGTACCGGCGGCCCGAGCCAGCCGGCGTGTCCGGGCGATGATGCGAGCATTXGATCTGXXTATXXXCTGXXTATCTGXGCGGTGGCGGGTGGTGAGCTCATTTT-3′ (SEQ ID NO: 30), where X represents a single trimer phosphoramides, flanked by introduced SacI site. The sense strand complements the 3′ end of the antisense strand to allow Klenow extension, 5′-AAAATGGTACCGGCGGCCCGAGCCAGCCGGCGTGTCCGGGCGATGATGCGAGCATT-3′ (SEQ ID NO: 31). The sense strand possesses introduced KpnI site. Annealed and extended Griptide DNA-library inserts together with the modified phagemid vector are digested with KpnI and SacI for 5 h at 37° C., and the digested products are purified using Monarch PCR and DNA cleanup kit (New England Biolabs, Ipswich, MA). The digested modified phagemid vector dephosphorylated using calf intestine phosphatase, CIP, (New England Biolabs, Ipswich, MA) and purified using Monarch PCR and DNA cleanup kit (New England Biolabs, Ipswich, MA). Purified DNA-library inserts and modified phagemid vector ligated by T4 ligase (New England Biolabs, Ipswich, MA). The resulted Griptide DNA-library-containing phagemid vector is transformed into E. coli electrocompetent TG1 cells (Lucigen, Middleton, WI) using established electroporation procedures. Following a recovery step, a portion of transformed TG1 containing Griptide DNA-libraries is serial diluted and plated to determine library diversity. The remaining recovered TG1 containing Griptide DNA-libraries are plated on LB-agar containing ampicillin (100 μg/mL) and glucose (2% w/v) overnight at 37° C. The following day, TG1 containing Griptide DNA-libraries scrapped from the plate into 2YT media containing 2% (w/v) glucose and subcultured in 62.5 mL 2YT containing ampicillin (100 μg/mL) and 2% glucose with shaking at 37° C. until OD₆₀₀ of 0.4 to 0.5. TG1 containing Griptide DNA-libraries superinfected with pIII-deficient helper phage, CM13d3 (Antibody Design Labs, San Diego, CA), at a MOI of 10 for 30 min infection without shaking at 37° C., followed by 30 min infection with shaking at 37° C. Infected TG1 containing Griptide DNA-libraries are pelleted to remove glucose and resuspended in 500 mL 2YT containing kanamycin (50 ug/mL), ampicillin (100 μg/mL) and 0.1 mM IPTG. Phage-displayed Griptide libraries are amplified by expanding the E. coli culture for 5 h with shaking at 30° C., pelleting E. coli cells at 5000×g, precipitating phage particles from the supernatant by addition of 1/5 volume of 20% (w/v) polyethylene glycol 8000, 2.5 M NaCl, followed by overnight incubation at 4° C., pelleting at 5000×g, and resuspending in Tris-buffered saline (TBS). Phage-displayed Griptide libraries are further purified by repeating the precipitation, pelleting, and resuspension steps. Purified phage-displayed Griptide libraries are stored as solutions in 50% (v/v) glycerol in TBS at −80° C. at >10¹² pfu/mL. Individual phage library members are characterized by Sanger sequencing followed by NGS. Well-separated colonies are picked from the library tittering plate in 20 μL of water. 2 μL of a resuspended template is mixed with 23 μL of the amplification master mix containing OneTaq DNA polymers (NEB, Ipswitch, MA) and two 10 uM phagemid sequence-specific amplification primers (Forward: 5′-GTGGAATTGTGAGCGGATAACAATTTG-3′ (SEQ ID NO: 32) and Reverse: 5′-GCGTAACGATCTAAAGTTTTGTCG-3′ (SEQ ID NO: 33)). Routine PCR is performed. Samples are submitted for standard Sanger sequencing (GENEWIZ, Cambridge, MA). Prior to library screening, deep sequencing (as described below) of the library is performed to ensure that it is high in sequence diversity and is not dominated by a small number of individual sequences, which can occur if construction is not performed properly.

Phage Screen: In some embodiments, phage screening is performed using biotinylated proteins bound to streptavidin magnetic beads (Dynabeads MyOne Streptavidin T1, ThermoFisher Scientific, Waltham, MA). 10¹⁰ phage particles are added to each phage screening sample, to ensure approximately 100 copies of each of the 10′ library members. Phage-display libraries are incubated with streptavidin magnetic beads for 1 h at room temperature in a buffer of 1×TBS, 1 mM MgCl2, 1% (w/v) BSA, 0.1% Tween-20, 0.02% (w/v) sodium azide, 5% (w/v) nonfat milk to remove bead-binding library members. For each screening condition, 100 μL of 2 uM biotinylated protein is captured with 0.5 mg of streptavidin-coated magnetic beads that have been previously blocked with 1% BSA, 0.1% Tween-20, 2% glycerol in 1×TBS pH 7.4 at room temperature for 15 min in 96-well plates, then the supernatant is removed using a plate magnet and the beads are promptly but gently resuspended in 50 μL of the same buffer. 150 μL of the depleted phage library is added to each well for 200 uL final volume, plates are sealed, and the screening reactions are incubated at room temperature for 45 min, with rotation to maintain beads in solution. Inspection of these solutions is performed to confirm that beads have not aggregated or crashed out of solution, which can be indicative of protein aggregation. Biochemical tests should be performed to ensure that target proteins are stable under the conditions of the screen, and conditions should be adjusted accordingly if required. Following binding, beads are washed 5 times with ice-cold 1×TBS, 1 mM MgCl2, 1% (w/v) BSA, 0.1% Tween-20, 0.02% (w/v) sodium azide, 2% (w/v) glycerol. This can be performed with an automated bead handler such as a KingFisher (Thermo Fisher) or manually. Target-bound phage library members are then directly processed for NGS.

Next Generation Sequencing: To sequence the phage-displayed Griptide library members, phage particles are denatured from beads at 95° C. for 15 min in 25 mM Tris pH 8, 50 mM NaCl, 0.5% Tween-20. Prior to boiling, 12,500 copies of a phage clone of known sequence (not a library member) are spiked in to each well to enable cross-well normalization of sequence reads. A two-step low-cycled PCR is performed to introduce Illumina adaptors and 10 bp TruSeq DNA UD Indexes (Illumina San Diego, CA) to the 3′ and 5′ ends of amplicons with modified phagemid vector forward and pADL-100 reverse primers (Forward: 5′-TCGTCGGCAGCGTCAGATGTGTATAAGAGACAGGGCGGATTATAAAGATGACGATGACAAAGG-3′ (SEQ ID NO: 34) and Reverse: 5′-GTCTCGTGGGCTCGGAGATGTGTATAAGAGACAGCGTTAGTAAATGAATTTTCTGTATGAGG-3′ (SEQ ID NO: 35)) similar to Illumina's 16S Metagenomic Sequencing Library Preparation protocol. The NGS library is sequenced by an Illumina NovaSeq platform using a 2×150 bp high-output kit (Illumina San Diego, CA).

Hit ID and Clustering: NGS reads are trimmed for quality (Phred score >18) and filtered for sequences that matched the design of the phage library. Counts for each unique sequence are tallied, and then normalized by the counts of the spike-in sequence added to each sample. A metric called Hit Strength is computed for each sequence as the fold change between the normalized counts in the highest target concentration sample and the normalized counts in the blank bead samples (averaged across experimental replicates). When 0 counts are observed for a sequence in blank bead samples, a count of 0.5 is used to prevent dividing by zero. Sequences with a hit strength greater than 5 are subjected to hierarchical clustering to identify sequence families. Pairwise distances between sequences i and j are computed using

$${distance}_{ij} = ( 1 - \frac{{score}_{ij}}{{score}_{ii}} ) ( 1 - \frac{{score}_{ij}}{{score}_{jj}} ) ,$$

where scoreᵢⱼ is the alignment scores based on a modified BLOSUM62 substitution matrix. Tryptophan-tryptophan match score is decreased from 11 to 7 in the BLOSUM62 substitution matrix to prevent overly-biasing clustering towards tryptophans. Hierarchical clustering using average linkage is used to group the sequences into families. To avoid clustering using a large number of sequences, which is computationally intensive and can make it difficult to identify small clusters, multiple rounds of clustering is performed. First, sequences are sorted by descending hit strength. The top 1000 (first round of clustering), top 2000 (2^{nd} round of clustering), or top 3000 (≥3^{rd} round of clustering) sequences, are then taken and are subjected to clustering as described above. Clusters of sequences with high sequence similarity (sequence “families”) are identified at each round and removed from the pool of sequence for subsequent rounds. Sequences subjected to 3 rounds of clustering without falling into a sequence family are similarly dropped from subsequent rounds of clustering under the assumption that they do not belong to a sequence family. The process is halted after 10 rounds of clustering, or when no sequences remained in the list.

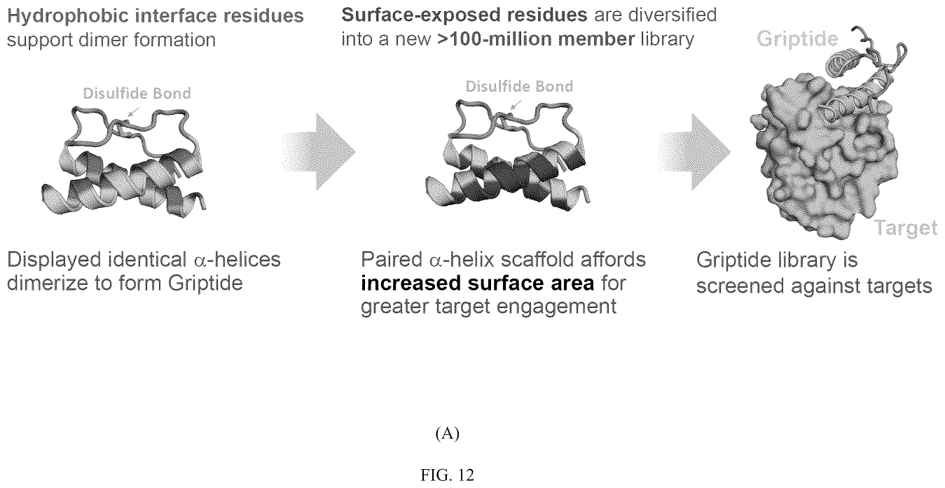

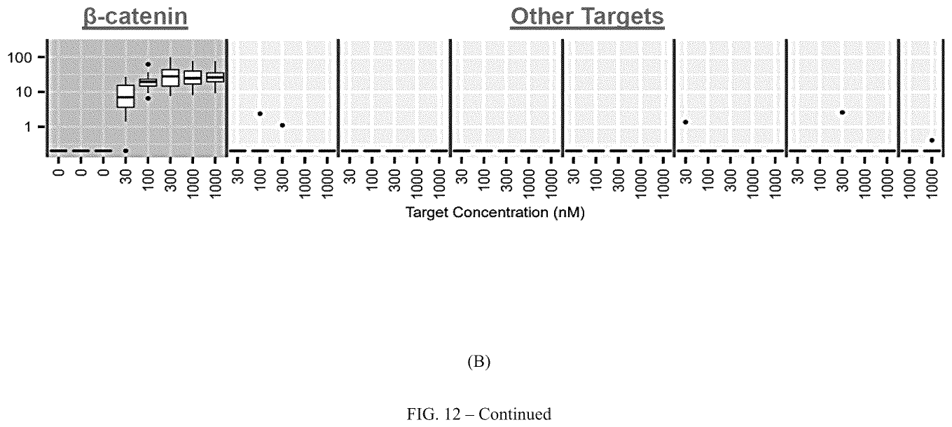

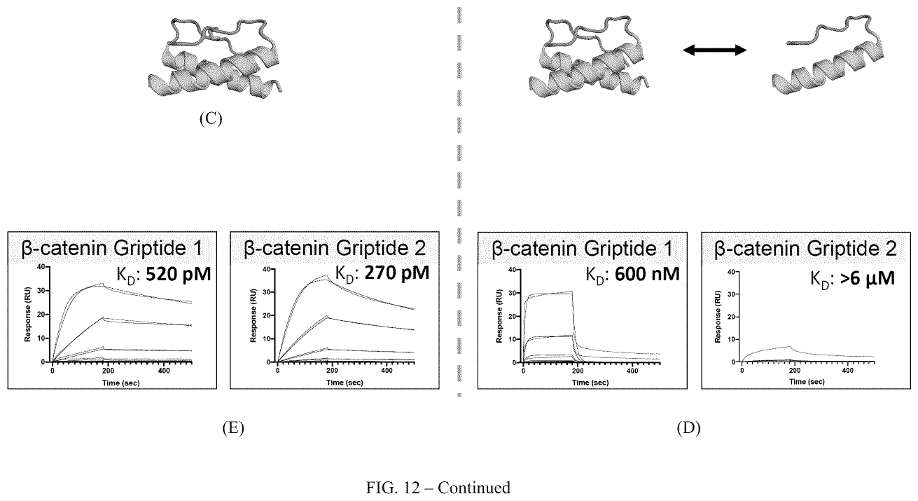

Certain useful technologies and data are described in FIG. 12 as examples. As demonstrated, griptides can provide high affinity and selectivity. In some embodiments, in a griptide there are two units of Gly-Pro-Ser-Gln-Pro-Ala-Cys-Pro-Gly-Asp-Asp-Ala-Ser-Ile-Leu-Asp-Leu-Gln-Leu-Tyr-Asp-Leu-Asp-Leu-Tyr-Asp-Tyr-Leu-Leu-Ala-Val-Ala, wherein the Cys of one unit is crosslinked with the Cys in the other (SEQ ID NO: 36). In some embodiments, in a griptide there are two units of Gly-Pro-Arg-Arg-Pro-Arg-Cys-Pro-Gly-Asp-Asp-Ala-Ser-Ile-Phe-Asp-Leu-Asp-Met-Tyr-Phe-Glu-Trp-Leu-Asp-Asp-Tyr-Leu-Thr-Ala-Val-Ala, wherein the Cys of one unit is crosslinked with the Cys in the other (SEQ ID NO: 37).

### Example 10. Certain Useful Technologies

Various technologies can be utilized to design, identify, characterize, manufacture, assess, and use provided technologies in accordance with the present disclosure. Certain technologies are described in U.S. Ser. No. 11/198,713, US 20210179665, WO 2021119537, WO 2021188659, WO 2022020651, or WO 2022020652, the entirety of each of which is incorporated herein by reference. Certain useful technologies are described below as examples.

Crosslinker Synthesis: N,N′-(1,4-phenylene)bis(2-bromoacetamide) was synthesized by adding bromoacetyl bromide (1.1 mmol) to a solution of p-phenylenediamine (0.5 mmol) in dichloromethane (DCM) (5 mL), dry pyridine (1.2 mmol), and 4-(dimethylamino)pyridine (13 umol) at 0° C. The mixture was stirred at 25° C. for 3 hours. The reaction mixture was filtered and washed with DCM to obtain N,N′-(1,4-phenylene)bis(2-bromoacetamide) as an off-white solid. ¹H NMR (DMF-d7, 500 MHz): δ 4.12 (s, 4H), 6 7.67 (s, 4H), 6 10.47 (s, 2H).

Helicon Synthesis and Cysteine Bisalkylation: Helicons were synthesized at 100 umol scale on Rink Amide resin (˜0.5 mmol/g) using standard Fmoc-based solid phase peptide synthesis workflows. Specifically, the Fmoc-protected resin was swollen using N,N-dimethylformamide (DMF) before coupling the first amino acid. Fmoc-deprotection was performed by treating the resin with 20% (v/v) piperidine in DMF. The amino acid coupling was performed in DMF with 4 equivalents of Fmoc-protected amino acid, 8 equivalents of ethyl cyanohydroxyiminoacetate (oxyma), and 4 equivalents of N,N′-diisopropylcarbodiimide (DIC). The peptides were globally deprotected and cleaved off the resin by treating the resin with a cleavage cocktail composed of 92.5% (v/v) trifluoroacetic acid (TFA), 2.5% (v/v) water, 2.5% (v/v) triisopropylsilane, and 2.5% (v/v) mercaptopropionic acid for 2 hours. The crude peptide was precipitated by adding ice-cold isopropyl ether to the concentrated cleavage cocktail. The precipitated peptide was pelleted by centrifugation and dried under nitrogen.

The crude peptide was dissolved in DMSO before proceeding with the cysteine bisalkylation/stapling reaction. The DMSO stock was diluted in a 2:1 solvent mixture of acetonitrile and 50 mM ammonium hydroxide. The pH of the solution was adjusted to ˜8.5 using N, N-Diisopropylethylamine (DIPEA). 1.3 equivalents of the alkylating agent, N, N′-(1,4-phenylene)bis(2-bromoacetamide) in DMF were added to the crude peptide solution. The reaction mixture was stirred at room temperature for at least two hours or until the reaction had been completed. The progress of the reaction was monitored by analytical HPLC and mass spectrometry. The final reaction was quenched by 0-mercaptoethanol before lyophilizing the reaction mixture. The lyophilized reaction mixture was dissolved in DMSO for purification.

The crude peptide mixtures were purified by preparatory HPLC (solvent A: water with 0.1% (v/v) FA; solvent B: acetonitrile with 0.1% (v/v) FA) using a C18 column. Analytical HPLC and mass spectrometry were used to characterize the peptides. The observed masses of certain final peptide products are presented in Table 1 as examples.

Circular Dichroism (CD) Spectroscopy: CD spectra of stapled and unstapled peptide pairs were obtained using Aviv Biomedical, Inc. 420 CD spectrometer with peptide concentrations at 25 uM or 50 uM in 20 mM phosphate buffer at pH=7.4. The CD measurements were obtained using a cuvette with a 1 mm pathlength at a fixed temperature of 25° C. Three scans were obtained at every 1-nm interval in the wavelengths ranging from 190 to 260 nm. The buffer background was subtracted from each CD spectrum, followed by smoothing the curves by the moving-means method with a convolution width of 2 data points. Next, the smoothed baseline between 250-260 nm was subtracted from the smoothed spectrum of the sample. Finally, the CD measurements were converted to mean residue molar ellipticity (deg·cm²·dmol⁻¹) for data visualization. As DPA residues were used as an N-terminal nonhelical cap, they were ignored for the mean residue molar ellipticity calculations. The percent helicity of peptides was calculated using the ratio of [θ]₂₂₂/[θ]ₘₐₓ. [θ]ₘₐₓ for the peptides was estimated to be −26964.28 using the formula below.

$${[ θ ]}_{max} = ( - 44000 + 250 T ) ( 1 - \frac{k}{n} )$$

$$T = 25 ° C . , k = 4 , n = 14$$

Phage Library construction (primers, protocol, crosslinking, and DNA sequencing): Phage-displayed peptide libraries were constructed using the filamentous bacteriophage vector M13KE (New England Biolabs, Ipswich, MA). Protocol guidelines in the New England Biolabs Ph.D.™ Peptide Display Cloning System kit were followed. Briefly, library of oligonucleotides were chemically synthesized using a mix of trimer phosphoramides (Glen Research, Sterling, VA) lacking cysteine, lysine, proline, and glycine, then annealed, extended, and ligated into a digested M13KE vector. The sense strand contains the library sequence, 5′-CATGCCCGGGTACCTTTCTATTCTCACTCTGCGGATCCGGCGXXXTGCXXGCAGCAXXTGTXXXGGTGGTTCTGGCTGGGGTCGTGGTTC-3′ (SEQ ID NO: 21), where X represents a single trimer phosphoramide incorporation, flanked by KpnI site. The antisense strand complements the 3′ end of the sense strand to allow Klenow extension, 5′-CATGTTTCGGCCGAACCACGACCTGCGCCAGAACCAC-3′ (SEQ ID NO: 22). The antisense strand possesses an EagI site. Annealed library inserts, along with the M13KE vector, were digested with EagI and KpnI for 5 hours, and the digested products were purified using Monarch PCR and DNA cleanup kit (New England Biolabs, Ipswich, MA), followed by T4 ligation (New England Biolabs, Ipswich, MA). The resulting library-containing vector was transformed into E. coli strain ER2738 (Lucigen, Middleton, WI) by electroporation, reserving and plating post-rescue to determine library diversity. Between 5×10⁷ and 1×10⁸ individual phage clones (established by a traditional plaque assay) were used for library amplification. Phages were amplified by adding the post-rescue electroporated cells to a robustly growing E. coli culture at early-log phase for 5 h in 500 mL LB media supplemented with 100 uM each of MgCl₂ and CaCl₂, with shaking at 37° C. E. coli cells were pelleting at 5000×g and supernatant was removed. Phage particles were collected from the supernatant by addition of 1/5 volume of 20% (w/v) polyethylene glycol 8000, 2.5 M NaCl, followed by overnight incubation at 4° C., pelleted at 5000×g, and resuspended in Tris-buffered saline (TBS). Phage-displayed Helicon libraries were further purified by repeating the precipitation, pelleting, and resuspension steps. After the final resuspension step, a plaque assay was performed to assess the overall titer of the phage display library. In some embodiments, between 5×10¹² and 1×10¹³ phage particles in each amplified library were observed.

Phage-displayed Helicon libraries were covalently crosslinked (stapled) by diluting the phage particle solution in TBS to an OD₆₀₀ of 1.0 and adding dithiothreitol to a concentration of 1 mM, followed by dialysis against 100 volumes of 20 mM NH₄CO₃, 2 mM EDTA, pH ˜8 for 30-60 min, followed by addition of the dialyzed phage to a solution of crosslinker prepared in 20 mM NH₄CO₃, 2 mM EDTA, pH ˜8 (final crosslinker concentration is 200 uM. As the crosslinker does not completely dissolve in buffer, the solution was sonicated immediately prior to mixing with phage to disperse the solid into a fine suspension) and incubation with rotation for 2 hours at 32° C. Excess crosslinker was removed first by pelleting at 5000×g and decanting, followed by addition of dithiothreitol to a concentration of 0.25 mM with incubation for 10 minutes, and then addition of iodoacetamide to a concentration of 0.75 mM with incubation for a further 10 minutes. Ellman's reagent (5,5′-dithiobis-(2-nitrobenzoic acid) was used to track the quenching of DTT until all thiols were capped. Phage particles were further purified by repeating the precipitation, pelleting, and resuspension steps described above for purification from E. coli culture, then are stored as solutions in 50% v/v glycerol in TBS at −80° C. at >10¹² pfu/mL. Next-generation sequencing was performed to assess the library quality (details can be found in the Phage NGS section). In some embodiments, between 10⁶-10⁷ phage particles were sequenced and found that on average, between 95%-98% of all reads have the correct library structure.

Mass spectrometry analysis of crosslinked phage was performed by adding 15 μL of phage samples to 2.5 uL of a solution of Trypsin at 1.0 mg/ml freshly prepared in 5 mM acetic acid and then pH adjusted by mixing 1:1 with 100 mM Tris pH 8. After 1 hour of digestion at room temperature, each cleavage reaction was quenched with 15 μL of 20% ACN+1% formic acid, and analyzed a Q-Exactive Plus mass spectrometer equipped with an Ultimate 3000 LC system (Thermo Electron) and a Aeris™ C18 column (Phenomenex). Finally, individual phage library members were characterized by DNA sequencing. Well-separated blue plaques were picked from the LB/IPTG/Xgal Agar plates in 50 μL of water. 2 μL of resuspended template was mixed with 23 μL of the amplification master mix containing OneTaq DNA polymers (NEB, Ipswitch, MA) and two 10 uM M13KE sequence-specific amplification primers (NEB, Ipswitch, MA). Routine PCR was performed, and samples were submitted for standard Sanger sequencing (GENEWIZ, Cambridge, MA). Prior to library screening, deep sequencing (as described below) of the library were performed to ensure that it is high in sequence diversity and is not dominated by a small number of individual sequences.

Phage Library Screening: Phage screening was performed using biotinylated proteins bound to streptavidin magnetic beads (Dynabeads MyOne Streptavidin T1, Thermo Fisher Scientific, Waltham, MA). About 10¹⁰ phage particles were added to each phage screening sample, to ensure approximately 100 copies of each of the 10⁸ library members. Phage display libraries were incubated with streptavidin magnetic beads for 1 hour at room temperature in a buffer made of 1×TBS, 1 mM MgCl₂, 1% w/v BSA, 0.1% Tween-20, 0.02% w/v sodium azide, 5% w/v nonfat milk to remove bead-binding library members. Briefly, Dynabeads were prepared in a 15 ml Falcon tube according to a manufacturing protocol from Thermo Fisher Scientific, diluted phage display libraries were added to the magnetic beads. After an incubation period, the tube was placed on a magnet for 1 min to separate bead-bound and non-bead-bound phage library members. Supernatant containing bead depleting phage library was collected and beads were discarded. For each screening condition, 100 μL of 2 uM biotinylated protein was captured with 0.5 mg of streptavidin-coated magnetic beads that have been previously blocked with 1% BSA, 0.1% Tween, 2% glycerol in 1×TBS pH 7.4 at room temperature for 15 minutes in 96-well plates, followed by removal of the supernatant using a plate magnet and prompt but gentle resuspension of the beads in 50 μL of the same buffer. Next, 150 μL of the depleted phage library was added to each well for 200 uL final volume, the plates were sealed, and the screening reactions incubated at room temperature for 45 minutes, with rotation to maintain beads in solution. These solutions was inspected to confirm that beads had not aggregated or crashed out of solution, which can be indicative of protein aggregation. Following binding, beads were washed 5× with ice-cold 1×TBS, 1 mM MgCl₂, 1% w/v BSA, 0.1% Tween-20, 0.02% w/v sodium azide, 2% w/v glycerol. Washing steps can be performed, as in our case, with an automated bead handler such as a KingFisher (Thermo Fisher) or manually. Target-bound phage library members are directly processed for NGS.

Phage Next-Generation Sequencing (NGS): In some embodiments, the protocol below is used to perform NGS for newly built phage display libraries and to identify target-bound phage library members after a phage screen. To sequence the phage-displayed peptide library members, phage particles are removed from the beads by a denaturation step at 95° C. for 15 min in 25 mM Tris pH 8, 50 mM NaCl, 0.5% Tween-20. Prior to boiling, 10,000 copies of a phage clone of known sequence (not a library member) are spiked in to each well to enable cross-well normalization of sequence reads. The sequence of the spike-in clone is TCTCACTCTGCGCCGGAATGCATTCTGGATTGCCATGTGGCGCGCGTGTGGGGTGGTTCT (SEQ ID NO: 38). A two-step low-cycled PCR is performed to introduce Illumina adaptors and 10 bp TruSeq DNA UD Indexes (Illumina, San Diego, CA) to the 3′ and 5′ ends of amplicons with M13KE Forward and M13KE Reverse primers (M13KE Forward: 5′-TCGTCGGCAGCGTCAGATGTGTATAAGAGACAGTTCGCAATTCCTTTAGTGG-3′ (SEQ ID NO: 25) and M13KE Reverse: 5′-GTCTCGTGGGCTCGGAGATGTGTATAAGAGACAGATTTTCTGTATGGGATTTTGCTAA-3′ (SEQ ID NO: 26)) similar to Illumina's 16S Metagenomic Sequencing Library Preparation protocol. The NGS libraries are sequenced by an Illumina NovaSeq platform using a 2x150-bp high-output kit (Illumina, San Diego, CA).

Hit ID and Clustering: NGS reads were trimmed for quality (Phred score >18) and filtered for sequences that matched the design of the phage library. Counts for each unique sequence were tallied, and then normalized by the counts of the spike-in sequence added to each sample. A metric called Hit Strength was computed for each sequence as the fold change between the normalized counts in the highest target concentration sample and the normalized counts in the blank bead samples (averaged across experimental replicates). When 0 counts were observed for a sequence in blank bead samples, a count of 0.5 was used to prevent dividing by zero. Sequences with a hit strength greater than 5 were then subjected to hierarchical clustering to identify sequence families. Pairwise distances between sequences i and j were computed using

$${distance}_{ij} = ( 1 - \frac{{score}_{ij}}{{score}_{ii}} ) ( 1 - \frac{{score}_{ij}}{{score}_{jj}} ) ,$$

where scoreᵢⱼ is the alignment scores based on a modified BLOSUM62 substitution matrix. Tryptophan-tryptophan match score is reduced from 11 to 7 in the BLOSUM62 substitution matrix to prevent overly biasing clustering towards tryptophans. Hierarchical clustering using average linkage was used to group the sequences into families. To avoid clustering using a large number of sequences, which is computationally intensive and can make it difficult to identify small clusters, multiple rounds of clustering were performed. First, sequences were sorted by descending hit strength. The top 1000 (first round of clustering), top 2000 (2^{nd} round of clustering), or top 3000 (>3^{rd} round of clustering) sequences, were taken and subjected to clustering as described above. Clusters of sequences with high sequence similarity (sequence “families”) were identified at each round and removed from the pool of sequence for subsequent rounds. Sequences subjected to three rounds of clustering without falling into a sequence family were similarly dropped from subsequent rounds of clustering under the assumption that they did not belong to a sequence family. The process was halted after 10 rounds of clustering, or when no sequences remained in the list.

beta-Catenin Surface Plasmon Resonance (SPR): SPR experiments were performed on a Biacore™ 8K (Cytiva) instrument at 25° C. Test peptides were diluted into running buffer (50 mM Tris pH 8.0, 300 mM NaCl, 2% glycerol, 0.5 mM TCEP, 0.5 mM EDTA, 0.005% Tween-20, 1% DMSO). Compounds were diluted to 10 uM or 1 uM and serially diluted 1:3 for seven concentrations and two blanks (7-point three-fold peptide dilution series with top concentration=10 mM). Biotinylated β-catenin residues 134-665 (Uniprot ID P35222) was immobilized to the active surface of the sensor chip for 25 seconds at 10 uL/min using the Biotin CAPture Kit, Series S (Cytiva) and compounds were injected over the reference and active surfaces for 180 seconds at 65 uL/min then allowed to dissociate for 400 seconds. Results were analyzed using the Biacore™ Insight Evaluation software, with double-referencing and fitted to a 1:1 binding affinity model.

beta-Catenin-TCF Competition by Fluorescence Polarization: Compounds at 10 mM in DMSO were serially diluted 1:3 in DMSO for a total of 11 concentrations using a Mosquito LV (SPT Labtech), then diluted 1000-fold in buffer (50 mM HEPES, pH 7.5, 125 mM NaCl, 2% glycerol, 0.5 mM EDTA, 0.05% v/v pluronic acid) in duplicate by the Mosquito LV (SPT Labtech) into a black polystyrene 384-well plate (Corning) (11-point three-fold peptide dilution series with top concentration=10 mM) Probe solution (10 nM full-length β-catenin (Uniprot ID P35222), mixed with 10 nM 5FAM-labeled TCF4 residues 10-53 (Uniprot ID Q9NQB0) peptide (FP04872) in buffer) was prepared and plated using the MultiDrop Combi (Thermo Fisher) for a total reaction pool of 40 mL. The plate was incubated and protected from light for 1 hour at room temperature prior to read. Reads were performed on a CLARIOstar plate reader (BMG Labtech) with excitation at 485 nm, emission at 525 nm, and cutoff at 504 nm. Data were fitted to a 1:1 binding model with Hill slope using an in-house script.

beta-Catenin-Axin Competition by Fluorescence Polarization: Compounds at 10 mM in DMSO were serially diluted 1:3 in DMSO for a total of 11 concentrations using the Mosquito LV (SPT Labtech), then diluted 1000-fold in buffer (50 mM HEPES, pH 7.5, 125 mM NaCl, 2% glycerol, 0.5 mM EDTA, 0.05% v/v pluronic acid) in duplicate by the Mosquito LV (SPT Labtech) into a black polystyrene 384-well plate (Corning). Probe solution (15 nM full-length β-catenin (Uniprot ID P35222), mixed with 20 nM FITC labeled fStAx-33 peptide (FP00013) in buffer) was prepared and plated using the MultiDrop Combi (Thermo Fisher) for a total reaction pool of 40 mL. The plate was incubated protected from light for 1 hour at room temperature prior to read. Reads were performed on a CLARIOstar plate reader (BMG Labtech) with excitation at 485 nm, emission at 525 nm, and cutoff at 504 nm. Data were fitted to a 1:1 binding model with Hill slope using an in-house script.

Measurement of the Cell Association of Helicons: A “Source” plate of 47 test compounds was prepared at a concentration of 1 mM in 90% DMSO in a 500 uL 96-well plate (2 replicates for each compound with 2 DMSO blanks). The 96-well format with one compound per well was maintained for all transfers throughout the protocol. A 2000 uL 96-well v-bottom plate (“Cells” plate) was used to dilute 1.25 uL of compounds from the Source plate into 500 μL of Expi293™ Expression Medium. Each well also received 500 μL of Expi293™ cells (Thermo Fisher) for a total concentration of 1×10⁶ cells/mL in 1 mL of Expi293™ Expression Medium. Baseline cell health at the time of compound addition was measured using CellTiter-Glo™ 2.0 Reagent (CTG) and a GloMax Discover reader (Promega). The Cells plate was incubated in an Infors HT Multitron Pro shaking incubator at 1000 rpm, 37° C., 8.0% CO₂, ˜55% humidity, for 22 hours, along with four plates of DI water to maintain humidity. After 22 hours, the cells were sampled again for post-incubation CTG analysis of cell health. CellTiter-Glo fold-change is calculated as the luminescence readout at T_{final} divided by the luminescence readout at Tᵢₙᵢₜₐₗ (time at which peptide was added). The cells were washed twice with 200 μL of Dulbecco's Phosphate Buffered Saline (DPBS) and transferred to a 500 uL 96-well “Final Assay” plate. After the second wash, the cells were resuspended in 80 μL of buffer (90% DPBS, 10% dimethyl sulfoxide (DMSO), 10 uM of a mixture of nonstapled 14-mer peptides with randomized sequences) in the Final Assay plate. Cell lysis was induced by the addition of 240 μL of ammonium hydroxide and 2 hours of shaking at 37° C. All solvents were removed via 23 hours in a SpeedVac vacuum concentrator (Thermo Fisher). The dried compounds and cell debris were resuspended using 180 μL of resuspension buffer (47.5% Acetonitrile (ACN) with 0.1% formic acid (FA), 47.5% H₂O with 0.1% FA, 5% DMSO) and shaking for 3 hours at 600 rpm. Once resuspended, the cell debris was separated from the resuspended compounds via centrifugation at 3220 rcf for 20 min. A portion of compound-containing supernatant from each well was transferred to a corresponding well in a 384-well plate for mass spectrometry analysis. Matching “cell-free” wells for all “cells” wells were plated in the same 384-well plate. The “cell-free” wells were prepared by adding 1 μL of 0.1 mM compounds from the Source plate to 19 μL of input buffer (47.5% ACN with 0.1% FA, 47.5% H₂O with 0.1% FA, 5% DMSO, 10 uM of a mixture of nonstapled 14-mer peptides with randomized sequences), then adding 2 μL of this 5 uM-dilution to 58 μL of input buffer in the 384-well mass spectrometry plate. All samples were analyzed using mass spectrometry. The percentage of compound in cells after treatment and wash was computed as the percentage of the total compound added to the Cells samples that was present in the cell fraction after removal of the extracellular media. Compounds were quantitated by mass spectrometry, and the signal in the cell-free samples were used as a single-point calibration curve to convert from signal intensity in the Cells sample to the percentage of the total amount added.

beta-Catenin protein production: beta-catenin protein (residues 134-665) with a N-terminal His6-yBBr-TEV tag (“HHHHHH” disclosed as SEQ ID NO: 39) was recombinantly expressed in E. coli BL21 (DE3) pLysS cells (Thermo Fisher) from pET28a vectors (Novagen). The cells were induced at OD₆₀₀=0.6 with 0.15 mM isopropyl P-D-1-thiogalactopyranoside (IPTG) for 16 hours at 16° C., then harvested and resuspended in 25 mM Tris pH 8.0, 200 mM NaCl, 10% glycerol, 20 mM imidazole, 1 mM PMSF. For purification, the pellet was lysed with a tip sonicator, pelleted at 22,000×g for 30 minutes at 4° C., then the supernatant was purified with HisTrap HP columns (Cytiva), eluting with 250 mM imidazole. For crystallography efforts, protein was TEV-cleaved by adding TEV protease at a ratio of 1:10 protease to protein and incubated overnight at 4° C. For biochemical assays, protein was biotinylated via the yBBr reaction according to standard procedures. All proteins were concentrated using Amicon spin filters (Millipore Sigma) then diluted into 25 mM Tris, pH 8.8, 1 mM DTT, 10% glycerol and loaded onto a Q HP (Cytiva) column. Proteins were eluted with a gradient from 50 mM to 600 mM NaCl. Protein-containing fractions were pooled, concentrated and loaded onto a Superdex® 10/300 200 pg (Cytiva) SEC column. Purified proteins were eluted isocratically in 25 mM Tris-HCl, pH 8.8, 10% glycerol, 300 mM NaCl and fractions containing pure protein were collected and pooled.

RNF31 Protein production (UBA, PUB, Sharpin). UBA Domain: RNF31 UBA domain protein (residues 480-639) with N-terminal Thioredoxin-TEV-6×his-yBBr-3C tags (“HHHHHH” disclosed as SEQ ID NO: 39) was recombinantly expressed in E. coli BL21 (DE3) pLysS cells (Thermo Fisher) from pET-derived expression vectors (Novagen). The cells were induced at OD₆₀₀=0.6 with 0.25 mM IPTG for 16 hours at 16° C., then harvested and resuspended in 50 mM HEPES pH 7.5, 500 mM NaCl, 15 mM imidazole, 1 mM TCEP, 2 mM ATP, 10 mM MgCl₂, 0.1× BugBuster®, 5% glycerol 25 U/mL Ready-Lyse™, 25 U/mL Omnicleave™, and 1 tablet Roche cOmplete™ EDTA-free per 50 mL. For purification, the pellet was lysed with a tip sonicator, pelleted at 22,000×g for 40 minutes at 4° C., then the supernatant was purified with HisTrap HP columns (Cytiva), eluting with 500 mM imidazole. For crystallography efforts, protein was cleaved by adding 3C protease at a ratio of 1:40 protease to protein and incubating overnight at 4° C. For biochemical experiments, protein was cleaved by adding TEV protease at a ratio of 1:10 protease to protein and incubating overnight at 4° C. overnight. TEV cleaved proteins were then biotinylated via the yBBr reaction. Proteins were concentrated and injected over a Superdex® HiLoad 16/600 75 pg SEC column pre-equilibrated with 50 mM HEPES pH 7.5, 150 mM NaCl, 1 mM TCEP and 5% Glycerol. Proteins were eluted isocratically and fractions containing pure protein were collected and pooled. PUB domain: RNF31 PUB domain protein (residues 1-179) with N-terminal MBP-TEV-6×His-YBBR-3C tags (“HHHHHH” disclosed as SEQ ID NO: 39) was recombinantly expressed in E. coli BL21 (DE3) pLysS cells (Thermo Fisher) from pET-derived expression vectors (Novagen). The cells were induced at OD₆₀₀=0.6 with 0.15 mM IPTG for 16 hours at 16° C., then harvested and resuspended in 50 mM HEPES pH 7.5, 500 mM NaCl, 1 mM TCEP, 2 mM ATP, 5 mM MgCl₂, 5% Glycerol, and 1 mM PMSF. For purification, the pellet was lysed with a tip sonicator, pelleted at 22,000×g for 30 minutes at 4° C., then the supernatant was purified with Ni-NTA resin (Qiagen), eluting with 250 mM imidazole. For crystallography efforts, protein was cleaved by adding 3C protease at a ratio of 1:40 protease to protein and incubating overnight at 4° C. For biochemical experiments, protein was cleaved by adding TEV protease at a ratio of 1:10 protease to protein and incubating overnight at 4° C. overnight. TEV cleaved proteins were then biotinylated via the yBBr reaction. Proteins were concentrated and injected over a Superdex® HiLoad 16/600 75 pg SEC column pre-equilibrated with 25 mM HEPES pH 7.5, 150 mM NaCl, 1 mM TCEP. Proteins were eluted isocratically and fractions containing pure protein were collected and pooled.

Sharpin: Sharpin ubiquitin-like domain protein (residues 206-309) with N-terminal Thioredoxin-6×His-Thrombin-yBBr-TEV tags (“HHHHHH” disclosed as SEQ ID NO: 39) was recombinantly expressed in E. coli BL21 (DE3) pLysS cells (Thermo Fisher) from pET-derived expression vectors (Novagen). The cells were induced at OD₆₀₀=0.6 with 0.2 mM IPTG for 16 hours at 16° C., then harvested and resuspended in 50 mM HEPES pH 7.5, 500 mM NaCl, 15 mM imidazole, 1 mM TCEP, 2 mM ATP, 10 mM MgCl2, 0.1× BugBuster®, 5% glycerol 25 U/mL Ready-Lyse™, 25 U/mL Omnicleave™, and 1 tablet Roche Complete EDTA-free per 50 mL. For purification, the pellet was lysed with a tip sonicator, pelleted at 22,000×g for 30 minutes at 4° C., then the supernatant was purified with HisTrap HP columns (Cytiva), eluting with 500 mM imidazole. For biochemical experiments, protein was cleaved by adding TEV protease at a ratio of 1:10 protease to protein and incubating overnight at 4° C. overnight. TEV cleaved proteins were then biotinylated via the yBBr reaction. Proteins were concentrated and injected over a Superdex® HiLoad 16/600 75 pg SEC column pre-equilibrated with 50 mM HEPES pH 7.5, 150 mM NaCl, 1 mM TCEP and 5% Glycerol. Proteins were eluted isocratically and fractions containing pure protein were collected and pooled.

RNF31 UBA and PUB SPR: All SPR experiments were performed on a Biacore 8K (Cytiva) instrument at 25° C. For kinetics experiments, the instrument was primed with 10 mM HEPES, pH7.5, 150 mM NaCl, 0.05% Tween 20, 1% DMSO. A CAP Series S sensor chip was docked and pre-conditioned with 3 injections of 1×CAP regeneration solution to remove unbound capture reagent from the surface. Biotinylated RNF31 UBA and PUB domain proteins were diluted to 1 uM in running buffer. FP06655 was diluted to 1 uM in running buffer and serially diluted 1:3 for a total of 8 concentrations and a blank (8-point four-fold peptide dilution series with top concentration=1 mM.). Otulin and test peptides were diluted to 10 uM in running buffer and serially diluted 1:3 for a total of 8 concentrations and a blank. Proteins were captured to the active surface of the sensor chip for 60 seconds at 5 μL/min and the peptides were injected over the reference and active surfaces for 180 seconds at 50 μL/min then allowed to dissociate for 360 seconds. Surface was regenerated with a 120-second injection of CAP regeneration solution each cycle. Data was analyzed using Biacore Insight Evaluation software (Cytiva). Sensorgrams were double-referenced and fit to 1:1 binding affinity model.

RNF31-Otulin competition. Fluorescence Polarization: RNF31 PUB domain was diluted to 1.6 uM in assay buffer (10 mM HEPES pH 7.4, 150 mM NaCl, 0.05% Tween 20) and pipetted into a 384-well black microplate (Corning) in a final volume of 20 uL. Test peptides were added to the plate (40 nL each) serially diluted 3-fold from 10 mM and the plate was incubated at room temperature for 20 minutes (11-point three-fold peptide dilution series with top concentration=10 mM). FITC-labeled Otulin peptide (residues 49-67) (FP16923) was diluted to 40 nM in assay buffer, then 20 μL of the stock was added to the plate for a final volume of 40 μL. The plate was incubated for 60 minutes at room temperature, then fluorescence anisotropy was recorded on a CLARIOstar (BMG LabTech) with excitation at 485 nm, emission at 525 nm, and cutoff at 515 nm. Data were plotted using Prism (Graphpad) and fit to a one-site specific binding model with Hill coefficient. SPR ABA Competition: All SPR experiments were performed on a Biacore 8K (Cytiva) instrument at 25° C. For kinetics experiments, the instrument was primed with 10 mM HEPES, pH 7.5, 150 mM NaCl, 0.05% Tween 20, 1% DMSO. A SA Series S sensor chip was docked and pre-conditioned with three injections of 50 mM NaOH/1M NaCl to remove unbound streptavidin from the surface. Biotinylated RNF31 PUB domains were diluted to 2 uM in running buffer. FP06649 and FP06652 were diluted to 10 uM in running buffer. Otulin peptide (residues 49-67) was diluted to 10 uM in running buffer. Proteins were captured on the active surface of the sensor chip for 300 seconds at 1 uL/min. For each injection, compounds were injected over the surface for 120 seconds at 30 uL/min to achieve equilibrium binding. Otulin was then injected for 60 seconds at 30 uL/min in the absence or presence of competing compound over the surface. Surface was regenerated with an injection of 1M sodium chloride after each cycle. Data was analyzed using Biacore Insight Evaluation software (Cytiva). Sensorgrams were double-referenced and evaluated for competition.

RNF31-Sharpin competition by fluorescence polarization: RNF31 UBA domain, and FAM-labeled RNF31-binding peptide, (FP12122), were diluted to 400 nM and 40 nM, respectively in assay buffer (10 mM HEPES pH 7.4, 150 mM NaCl, 0.05% Tween 20) and pipetted into a 384-well black microplate (Corning) in a final volume of 20 uL. Recombinant Sharpin/SIPL1 UBL protein (residues 153-256), as well as selected control peptides, were added to the plate (20 mL each), serially diluted 3-fold from 10 mM (10-point three-fold peptide or Sharpin dilution series with top concentration=3.3 mM). The plate was incubated for 60 minutes at room temperature, then fluorescence anisotropy was recorded on a CLARIOstar (BMG LabTech) with excitation at 485 nm, emission at 525 nm, and cutoff at 515 nm. Data were plotted using Prism (Graphpad) and fit to a one-site specific binding model with Hill coefficient.

CDK2 and PPIA protein production. CDK2: Full length CDK2 (residues 1-298) with an N-terminal GST-3C-6×his-TEV tag (“HHHHHH” disclosed as SEQ ID NO: 39) was recombinantly expressed in Sf9 cells according to the Bac-to-Bac protocol (Thermo Fisher). Briefly, Sf9 cells were plated at 1×10⁶ cells in 2 mL Sf-900™ II media (Thermo Fisher) into a 6-well cell culture plate. Cells were transfected with purified bacmid diluted in OptiMem™ media using Cellfectin™ reagent. Cells were incubated at 27° C. for 5 days. Cells and supernatant were removed from plate and centrifuged. P1 virus was collected and cell pellet was evaluated for protein expression by Western blot. P2 virus was generated by infecting 2×10⁶ cells/mL Sf9 in 50 mL Sf-900™ II media with 500 mL P1 virus. Cells were incubated with shaking at 27° C. for 5 days. Cells were centrifuged at 1500 rpm at room temperature for 5 minutes. Supernatant was stored at 4° C. as P2 virus stock and pellet was evaluated by Western blot for protein expression. Protein was expressed by seeding Sf9 cells at 2×10⁶ cells/mL in Sf-900™ II media and infecting at an MOI of 1:200. Cultures were incubated with shaking for 72 hours at 27° C. Cells were harvested and supernatant was discarded. Pellets were resuspended in 25 mM HEPES, pH 7.5, 300 NaCl, 10% glycerol, 0.5 mM PMSF and then sonicated with a tip sonicator. Lysates were centrifuged at 22,000×g for 30 minutes at 4° C. Clarified lysate was purified with a GSTrap™ (Cytiva) pre-equilibrated in 25 mM HEPES, pH 7.5, 300 mM NaCl, 10% glycerol. Protein was eluted with 25 mM HEPES, pH 7.5, 300 mM NaCl, 10% glycerol, 10 mM GSH. Eluted protein was cleaved by combining protein with TEV protease at a ratio of 1:10 protease to protein and incubating at room temperature for 40 hours. Cleaved protein was then dialyzed into 25 mM HEPES, pH 7.5, 300 mM NaCl, 10% glycerol and re-injected over a GSTrap™ to remove cleaved tags. Purified protein was concentrated and centrifuged at 22,000×g for 10 minutes at 4° C. to remove soluble aggregates. Protein was then loaded onto a Superdex™ HiLoad 16/600 75 pg SEC column pre-equilibrated with 20 mM HEPES, pH 7.5, 150 mM NaCl, 2% glycerol, 2 mM DTT. Protein was eluted isocratically at 0.5 mL/min. Finally, protein was centrifuged at 22,000×g for 10 minutes at 4° C. to remove soluble aggregates, then aliquoted and frozen.

CDK2 (pT160): CDK2 with T160 phosphorylation was obtained through the co-expression of GST-3C-6×his-TEV-CDK2 (“HHHHHH” disclosed as SEQ ID NO: 39) above with Saccharomyces cerevisiae GST-Cak1. The phosphorylation of pT160 was confirmed with phospho-CDK2 (Thr160) antibody (Cell Signaling #2561) and mass spectrometry.

CDK2 (pT160)/CCNE1: GST-CCNE1 (residues 81-363) was expressed in E. coli BL21 (DE3) pLys S cells. Briefly, cells were grown at 37° C. to OD₆₀o reached 0.8 and induced with 0.1 mM IPTG overnight at 20° C. Cells were lysed in 10 mM HEPES, 150 mM NaCl, pH 7.5 and pelleted with centrifugation. The cell lysate was incubated with glutathione-Sepharose 4B beads with purified CDK2 harboring pT160. After elution with 20 mM glutathione, pCDK2/GST-cyclin E1 was digested with GST-3C protease overnight. The pCDK2/cyclin E1 complex was collected and loaded onto a Superdex™ 16/600 75 pg (Cytiva) SEC column pre-equilibrated with 20 mM Tris pH 7.4, 200 mM NaCl, 10% glycerol, 1.0 mM DTT. Purified proteins were eluted isocratically. Protein fractions were collected, concentrated, aliquoted and frozen.

PPIA: Full length PPIA (residues 1-165) with an N-terminal 6×his-yBBR-TEV tag (“HHHHHH” disclosed as SEQ ID NO: 39) was recombinantly expressed in E. coli BL21 (DE3) CodonPlus RIPL cells (Agilent) from pET-derived expression vectors (Novagen). The cells were induced at OD₆₀₀=0.6 with 0.15 mM isopropyl P-D-1-thiogalactopyranoside (IPTG) for 16 hours at 16° C., then harvested and resuspended in PBS pH 7.4 with 1 mM PMSF. For purification, the pellet was lysed with a tip sonicator, pelleted at 22,000×g for 30 minutes at 4° C. Supernatant was collected then centrifuged again at 22,000×g for 30 minutes at 4° C. The supernatant was purified with Ni-NTA resin (Qiagen), eluting with 250 mM imidazole. Protein was TEV cleaved by adding TEV protease at a ratio of 1:10 protease to protein and incubated for 4 hours at 4° C. Protein was then concentrated and diluted into 20 mM HEPES pH 7.0, 5% glycerol and centrifuged at 22,000×g for 10 min at 4° C. The supernatant was loaded onto a SP HP (Cytiva) column pre-equilibrated with 20 mM HEPES pH 7.0, 5% glycerol. Purified protein was eluted with a gradient from 0 mM to 1 mM NaCl. Protein fractions were pooled, concentrated then centrifuged at 22,000×g for 10 min at 4° C. Supernatant was collected and loaded onto a Superdex™ 16/600 75 pg (Cytiva) SEC column pre-equilibrated with PBS pH 7.4. Purified proteins were eluted isocratically in PBS pH 7.4. Protein fractions were collected, concentrated, aliquoted and frozen.

SPR for CDK2 and the CDK2/CCNE1 complex: SPR experiments were performed on a Biacore S200 or 8K (Cytiva) instrument at 25° C. in 1×HBS-P+ buffer (Cytiva) with 1% DMSO. A SA Series S sensor chip was docked and pre-conditioned with three injections of 50 mM NaOH/1 M NaCl to remove unbound streptavidin from the surface. CDK2 protein, or CDK2: CCNE1 complex, was diluted to g/mL in running buffer and immobilized to channels 1 through 8 at 5 uL/min for 50-80 seconds for a final immobilization level of ˜1800 RU. Peptides were diluted to 5 uM in running buffer then serially diluted 2-fold for a total of seven concentrations with one blank (7-point two-fold peptide dilution series with top concentration=5 uM.). Compounds were injected over the immobilized and reference surfaces at 30 uL/min for 60 seconds and then allowed to dissociate for 180 seconds without surface regeneration. Sensorgrams were double-referenced and fit to a 1:1 steady state affinity model.

ATP competition of CDK2: For ATP competition experiments, 50 mM of selected CDK2-binding compounds were added into 20 nM Bodipy-ATP- yS (Thermo Fisher) and 2 mM CDK2 in an assay buffer contained 20 mM Tris pH 8, 300 mM NaCl, 10% (v/v) glycerol, 2 mM TCEP, and 10 mM MgCl₂, and pipetted into a 384-well black microplate (Corning) in a final volume of 40 μL. The plate was incubated for 60 minutes at room temperature, then fluorescence anisotropy was recorded on a CLARIOstar (BMG LabTech) with excitation at 485 nm, emission at 525 nm, and cutoff at 515 nm. Final data were normalized against DMSO control and Bodipy-ATP-yS-free control.

SPR for PPIA, including cyclosporine competition: All SPR experiments were performed on a Biacore 8K (Cytiva) instrument at 25° C. For kinetics experiments, the instrument was primed with 10 mM HEPES, pH 7.5, 150 mM NaCl, 0.05% Tween 20, 1 mM DTT, 1% DMSO. A SA Series S sensor chip was docked and pre-conditioned with 3 injections of 50 mM NaOH/1 M NaCl to remove unbound streptavidin from the surface. PPIA protein was diluted to 5 μg/mL in running buffer and immobilized to channels 1 through 8 at 5 uL/min for 50 seconds for a final immobilization level of -1900 R U. Peptides were diluted to 5 uM in running buffer then serially diluted 2-fold for a total of seven concentrations with one blank (7-point two-fold peptide dilution series with top concentration=5 uM.). Compounds were injected over the immobilized and reference surfaces at 30 uL/min for 60 seconds then allowed to dissociate for 180 seconds. The surface was regenerated after each cycle with an injection of 1 M sodium chloride. Sensorgrams were double-referenced and fit to a 1:1 steady state affinity model. For ABA competition experiments, PPIA was immobilized similarly to a level of -850 RU. Compounds were diluted to 10 uM in running buffer and cyclosporine A (CsA) was diluted to 100 nM in running buffer. For each injection, peptides were injected over the surface for 120 seconds at 30 uL/min to achieve equilibrium binding. CsA was then injected for 60 seconds at 30 uL/min in the absence or presence of competing compound over the surface. Surface was regenerated after each cycle with an injection of 1 M sodium chloride. Data was analyzed using Biacore Insight Evaluation software (Cytiva). Sensorgrams were double-referenced and evaluated for competition.

PPIA inhibition assay: PPIA inhibition assays were performed at Eurofins Discovery (Ongar, Essex, United Kingdom). Briefly, a 1.5 mL of assay buffer (35 mM HEPES pH 7.8, 50 mM DTT) is pipetted into a 3-mL glass cuvette and cooled to 10° C. with stirring. Test compounds are diluted in 100% DMSO then added to the buffer to establish a blank. PPIA is then added at a final concentration of 2 nM and substrate is added to a final concentration of 60 uM. The absorbance at 330 nm is measured for 300 seconds. The resulting data were fit to a first order rate equation and the catalytic rate was calculated. An exponential curve was generated using the catalytic rate versus the inhibitor concentration to obtain a Ki value. CsA is included at a single concentration as a positive control.

Production of PDL1. E. coli Protein: For crystallography, human PD-L1 protein (residues 18-134) with a C-terminal 6×His tag (SEQ ID NO: 39) was recombinantly expressed in E. coli BL21 (DE3) CodonPlus RIPL cells (Agilent) from pET-derived expression vectors (Novagen). The cells were induced at OD₆₀₀=0.6 with 1.0 mM IPTG for 4 hours at 37° C., then harvested and resuspended in 20 mM Tris-HCl pH 8.0, 300 mM NaCl, 10% glycerol, 1 mM PMSF, DNase I. For purification, the pellet was lysed with a tip sonicator, pelleted at 22,000×g for 50 minutes at 4° C. Protein was located in the inclusion bodies, so the cell pellet was collected and resuspended in 50 mM Tris-HCl pH 8.0, 200 mM NaCl, 10 mM EDTA, 10 mM P-mercaptoethanol (f-ME), 0.5% Triton X-100 and stirred by magnetic stir bar at room temperature for 30 minutes. The suspension was centrifuged at 22,000×g for 30 minutes at 4° C. The process was repeated three times. The pellet was resuspended in 50 mM Tris-HCl pH 8.0, 200 mM NaCl, 10 mM EDTA, 10 mM f-ME and stirred by magnetic stir bar at room temperature overnight. The suspension was centrifuged at 22,000×g for 30 minutes at 4° C. and the supernatant was collected. Supernatant was loaded onto a HisTrap (Cytiva) pre-equilibrated with 50 mM Tris-HCl pH 8.0, 200 mM NaCl, 8 M urea, 10 mM f-ME and eluted with 250 mM imidazole. Protein was refolded by diluting into 100 mM Tris-HCl pH 8.0, 1 M L-Arginine, 0.235 mM GSH, 0.25 mM GSSG with incubation overnight at 4° C. Protein was then dialyzed against PBS pH 7.4 for 4 hours at 4° C. Dialysis was repeated three times. Protein was concentrated and injected onto a Superdex™ HiLoad 16/600 75 pg SEC column pre-equilibrated with 10 mM Tris-HCl pH 8.0, 20 mM NaCl. Fractions containing pure protein were collected and pooled. Mammalian Protein :For biochemical assays, human PD-Li (residues 18-239) with a C-terminal human IgG Fc-Avi™-tag or a C-terminal TEV-10×his-Avi™ tag (“HHHHHHHHHH” disclosed as SEQ ID NO: 40) were recombinantly co-expressed with BirA in Expi293™ cells from pcDNA-derived plasmid (Thermo Fisher) using the Expifectamine™ 293 expression system. For Fc-tagged protein, cells were harvested after 5 days of expression and supernatant was collected and passed over a MabSelect sure affinity column (Cytiva) pre-equilibrated with PBS pH 7.4. Protein was eluted with 100 mM sodium citrate, pH 3.0 and neutralized in 1M Tris-base, pH 9.0. Elution was concentrated and injected over a Superdex™ HiLoad 16/600 200 pg pre-equilibrated with PBS pH 7.4. Fractions containing pure protein were collected and pooled. For 10×his-tagged (SEQ ID NO: 40) protein, cells were harvested after 5 days of expression and supernatant was collected and passed over a Ni-NTA affinity column (Qiagen) pre-equilibrated with PBS pH 7.4. Protein was eluted with 300 mM imidazole. Elution was concentrated and injected over a Superdex™ HiLoad 16/600 75 pg pre-equilibrated with PBS pH 7.4. Fractions containing pure protein were collected and pooled.

SPR for PDL1, including PD1 competition: All SPR analysis was performed on a Biacore S200 (Cytiva) in at 25° C. BMS PD-Li interacting small molecules (BMSpep-57, BMS-1, BMS-1001) were obtained from MedChem Express. A Protein G Series S Sensor Chip (Cytiva) was docked into the instrument primed with PBS pH 7.4 with 0.05% Tween 20 and 1% DMSO. PD-L1-Fc was diluted in running buffer to 50 nM and captured on the surface for 60 seconds at 5 uL/min. Compounds were diluted to 2 uM then serially diluted 3-fold in running buffer (7-point two-fold dilution series with top concentration=5 mM (FP30790), or 6-point two-fold dilution series with top concentration=2 mM (others)). Diluted compounds were injected over the surface at 30 uL/min for 180 seconds and allowed to dissociate for 360 seconds. The surface was regenerated every cycle with a 60 second injection of 10 mM glycine-HCl, pH 2.5. The resulting sensorgrams were double-referenced and fit to a 1:1 binding model using Biacore Insight Evaluation software (Cytiva). For ABA competition experiments, PD-L1 was immobilized on a Streptavidin Series S Sensor Chip (Cytiva) to -250 RU. Compounds were diluted to 10 uM in running buffer and PD-1 was diluted to 400 nM in running buffer. For each injection, compounds were injected over the surface for 120 seconds at 30 uL/min to achieve equilibrium binding. PD-1 was then injected for 60 seconds at 30 uL/min in the absence or presence of competing compound over the surface. The surface was regenerated every cycle with a 60-second injection of 10 mM glycine-HCl, pH 2.5. Data was analyzed using Biacore Insight Evaluation software (Cytiva). Sensorgrams were double-referenced and evaluated for competition.

ELISA for PDL1: PD-1/PD-L1 ELISA competition assays were performed according to manufacturer's instruction (Acro Biosystems). Human PD-L1 was diluted to 2 μg/mL in PBS+0.05% Tween 20. High-binding 96-well plates (Corning) were coated with PD-L1 at 2 μg/100 uL per well. Human PD-1-Avi was diluted to 0.6 μg/mL in ELISA wash buffer (PBS+0.05% Tween 20+0.5% BSA+0.09% DMSO) and added to the coated wells to form complexes. Test peptides were diluted in ELISA wash buffer to 20 uM then serially diluted 4-fold and added to the PD-L1/PD-1-avi complexes. After incubation with the ligand, the plate was washed, and the bound ligand was detected with the addition of streptavidin-HRP, followed by development with 1-Step™ Ultra TMB-ELISA substrate solution (Thermo Fisher). The HRP reaction was stopped by adding ELISA stop solution (Thermo Fisher). Absorbance at 450 nm was determined on a CLARIOstar plate reader. Samples were blank-subtracted and normalized using in-plate controls. Data were plotted using Prism (Graphpad) and fit to a one-site specific binding model with Hill coefficient.

PD-L1 Dimerization Assays, analytical SEC and TR-FRET. Analytical SEC: All analytical SEC methods were performed at Viva Biotech (Shanghai, China) on an Agilent Bio-1260 Infinity II HPLC system. Complexes were prepared by mixing PD-L1 and peptide at a 1:2 protein to peptide ratio. Complexes were injected in separate analyses onto a Superdex® Increase 5/150 200 pg column pre-equilibrated with 10 mM Tris-HCl pH 8.0, 20 mM NaCl containing 1 uM of corresponding peptide (for complexes) or no peptide (for apo-protein). Data was processed using Agilent ChemStation software to determine retention time shifts. TR-FRET: For TR-FRET dimerization experiments, biotinylated, mammalian PD-L1 was diluted to 50 nM, Alexa Fluor™ 488 labeled PD-L1 was diluted to 120 nM and Terbium-labeled streptavidin (Cis-Bio) was diluted to 20 nM in assay buffer (10 mM HEPES pH 7.5, 150 mM NaCl, 0.05% Tween20) in a final volume of 40 μL per well of a black 384-well plate (Costar). Compounds were serially diluted in 90% DMSO and 80 nL of compound (11-point three-fold peptide dilution series with top concentration=20 mM) was added to the plate and the samples were incubated for 60 minutes at room temperature. FRET signal was determined using a LanthaScreen™ filter on a PheraStar (BMG Biotech) plate reader (Ex: 337 nm; Em1: 490 nM; Em2: 520 nM). The ratio of Em₅₂₀ to Em₄₉₀ was calculated and plotted against compound concentration. Resulting data was fit to a four-parameter dose-response curve with variable slope.

Crystallography. X-Ray Crystallography and Structure Determination. beta-Catenin/FP01567 complex crystals were obtained in 0.1 M Sodium phosphate monobasic monohydrate, 0.1 M Potassium phosphate monobasic, 0.1 M MES monohydrate pH 6.5, 2.0 M Sodium chloride. beta-Catenin/FP05874 complex crystals were obtained in 0.05 M MgCl₂, 0.1 M MES 6.5 5% (w/v) PEG 4000. RNF31/FP06649 complex crystals were obtained in 0.03 M Magnesium chloride hexahydrate; 0.03 M Calcium chloride dihydrate, Buffer System 1 pH 6.5 (0.0555M MES, 0.0445 Imidazole), 12.5% MPD, 12.5% PEG 1000, 12.5% PEG 3350. RNF31/FP06652 complex crystals were obtained in 0.02M DL-Glutamic acid monohydrate, 0.02 M DL-Alanine, 0.02 M Glycine, 0.02 M DL-Lysine monohydrochloride, 0.02 M DL-Serine, Buffer System 1 pH 6.5 (0.0555 M MES, 0.0445 M Imidazole), 20% v/v PEG 500 MME, 10% w/v PEG 20000. RNF31/FP06655 complex crystals were obtained in 10% w/v glycerol, 20% w/v ethanol. CDK2/FP19711 complex crystals were obtained in 0.1 M Tris pH 8.5, 25% (w/v) PEG 2,000 MME. CDK2/FP24322 complex crystals were obtained in 0.1 M Bicine pH 9.2, 2% (v/v) 1,4-Dioxane, 6% (w/v) PEG 20000. PPIA/FP29092 complex crystals were obtained in 0.15 M Potassium bromide, 30% (w/v) Polyethylene glycol monomethyl ether 2,000. PPIA/FP29102 complex crystals were obtained in 0.01 M Nickel (II) chloride hexahydrate, 0.1 M Tris pH 8.5, 20% (w/v) Polyethylene glycol monomethyl ether 2,000. PDL1/FP28135 complex crystals were obtained in 1.1 M Sodium malonate pH 7.0, 0.1 M HEPES pH 7.0, 0.5% (v/v) Jeffamine ED-2001 pH 7.0. PDL1/FP30790 complex crystals were obtained in 0.1 M Sodium acetate pH 4.6, 2.0 M Sodium formate. PPIA/FP29103 complex crystals were obtained in 0.2 M Sodium acetate, 0.1 M Sodium cacodylate pH 6.5, 30% (w/v) PEG 8000. PDL1/FP28132 complex crystals were obtained in 0.1 M MES pH 6.5, 1.6 M Magnesium sulfate. PDL1/FP28136 complex crystals were obtained in 0.1 M TRIS pH 8, 25% (v/v) PEG 350 MME.

Crystals were obtained by either the sitting hanging drop or hanging drop vapor diffusion methods at room temperature. Crystals were cryo-protected followed by flash-freezing in liquid nitrogen. Diffraction datasets were collected at 100K. Data was processed in XDS & XSCALE, AIMLESS, and/or STARANISO, all part of the autoPROC suite. Molecular replacement solutions were obtained using PHASER with previously deposited high resolution PDB structures as search models. Complete models were built through iterative cycles of manual model building in COOT and structure refinement was carried out using either REFMAC or PHENIX. Structure model figures were prepared using PyMOL (The PyMOL Molecular Graphics System, Version 2.4, Schrödinger, LLC.). Certain atomic coordinates and structure factors are in the Protein Data Bank, www.pdb.org. (e.g., PDB IDs: 7UWI, 7UWO, 7UX5, 7UXI, 7UXJ, 7UXK, 7UXM, 7UXN, 7UXO, 7UXP, 7UXQ, 7UY2, 7UYJ, and 7UYJ).

### Example 11. Provided Technologies for Recognition and Reprogramming of Polypeptide Surfaces

In some embodiments, the present disclosure provides technologies (e.g., agents, methods, etc.) for modulating interactions between polypeptides. In some embodiments, the present disclosure provides technologies for inducing interactions between two polypeptides. In some embodiments, two polypeptides do not interact with each other absence of technologies described herein, e.g., agents such as stapled peptide agents. In some embodiments, two polypeptide interact each other absence of technologies described herein but provided technologies provide new interactions, e.g., between and among residues, domains, surfaces, etc., that do not interact, and/or interact in different ways, absence of provided technologies. In some embodiments, two polypeptides interact each other absence of technologies described herein but provided technologies enhance one or more interactions, in some cases create one or more interactions, and/or reduce one or more interactions, in some cases remove one or more interactions, between and among residues, domains, surfaces, etc., of the polypeptides. In some embodiments, the provided technology provides technologies for recognition of polypeptide surfaces. In some embodiments, the present disclosure provides technologies for reprogramming polypeptide surfaces. In some embodiments, a polypeptide is or comprises an E3 ligase or a characteristic portion thereof.

Agents that modulate interactions or induce novel interactions between proteins are useful for various purposes including the study of biological systems, therapeutics uses, etc. In many cases, their discovery can be limited by the complexities of rationally designing interactions between three components. Technologies reported by others typically require known binders to each protein to inform initial designs. In some embodiments, the present disclosure provides general technologies for developing agents, in some embodiments, a-helically constrained (Helicon) polypeptides, that cooperatively induce the interaction between two target proteins without a requirement for previously known binders or an intrinsic affinity between the proteins in the absence of such agents. In some embodiments, such agents are or comprise stapled peptides. For example, in some embodiments, the present disclosure provides agents, e.g., Helicons, that are capable of binding every major class of E3 ubiquitin ligases, which are of great biological and therapeutic interest but remain largely intractable to targeting by small molecules. In some embodiments, the present disclosure provides technologies for developing agents and such agents that can induce interactions between polypeptides and E3 ubiquitin ligases. In some embodiments, the present disclosure provides phage-based screening technologies for developing trimerizers, e.g., trimerizer Helicons, that can induce interactions between polypeptides and E3s. In some embodiments, the present disclosure provides technologies that reprogram E3s to cooperatively bind a target polypeptide. In some embodiments, a target polypeptide is or comprises an enzyme (e.g., PPIA). In some embodiments, the present disclosure provides technologies for reducing levels of a target polypeptide, comprising contacting the target polypeptide with an agent that can induce interactions between the target polypeptide and an E3 ubiquitin ligase. In some embodiments, a target polypeptide is or comprises a transcription factor (e.g., TEAD4). In some embodiments, a target polypeptide is or comprises a transcriptional coactivator (e.g., β-catenin).

In some embodiments, polypeptide-polypeptide interactions (PPIs), e.g., protein-protein interactions, play a central role in many biological processes, e.g., binding of protein or peptide ligands to their receptors, modulation of activity and specificity of polypeptides such as enzymes, scaffolding of signaling cascades and other functional complexes, etc., which can become dysregulated in various conditions, disorders or diseases. Among other things, the present disclosure provides technologies for modulating PPIs.

Historically, efforts have largely focused on agents that disrupt PPIs. Reports has been made in recent years to induce the formation of novel PPIs, including in the context of reprogramming E3 ubiquitin ligases to recognize novel substrate proteins and mark them for proteasomal degradation. However, a key constraint facing many reported technologies, e.g., rational design of molecules that induce novel interactions between proteins, is the typical requirement to possess known binding ligands to each, or a known interaction between the two proteins or closely related relatives, to serve as a starting point for designs. Because many proteins cannot be effectively bound by small molecules (the “druggability” problem), this constraint significantly limits the proteins for which small molecule-based PPI-inducing agents can be developed. This limitation is particularly acute for E3 ubiquitin ligases, of which only a handful can be bound by small molecules. There has been reports of peptide-based solutions to modulating therapeutically relevant PPIs, given their ability to engage significantly larger surfaces than small molecules. A critical challenge in these efforts has been the delivery of peptides, which generally possess large numbers of exposed polar and charged groups, into cells.

Among other things, in some embodiments, the present disclosure provides technologies that overcome existing limits and challenges. In some embodiments, the present disclosure provides technologies, e.g., stapled peptide agents and uses thereof, that can target various polypeptides including those that cannot be effectively targeted by reported technologies. In some embodiments, agents of the present disclosure comprise a-helical structure. In some embodiments, agents of the present disclosure comprise a-helical structure reinforced, e.g., by stapling. In some embodiments, reinforced a-helical structures increase agents' ability to cross cellular membranes. In some embodiments, technologies of the present disclosure, e.g., stapled peptide agents, provide increased stability and membrane permeability, and are capable of binding large, flat protein surfaces that are inaccessible to targeting by small molecules. In some embodiments, technologies of the present disclosure, e.g., stapled peptide agents, with their expanded surface area and ability to engage surfaces that small molecules cannot, can induce PPIs, in some cases, novel PPIs.

A general method for developing useful agents, e.g., Helicons that cooperatively lead to the interaction between two proteins, which may be referred to as “trimerizer” Helicons, in some cases without relying on previously known binders to either, is described below as an example. In some embodiments, provided technologies, which are accessible to laboratories with standard molecular biology and DNA sequencing technologies, can be utilized to develop trimerizer Helicons that induce the binding of E3 ligases to target proteins, including for which they have no intrinsic affinity, thereby reprogramming their surfaces. Among other things, the present example confirms various technical effects, including benefits and advantages, of provided technologies.

It is reported that there are an estimated 600 human E3s, in four sub-families. Only a small number of these have been reported to be successfully co-opted into novel PPIs for applications of targeted protein degradation (TPD), using small molecules called “molecular glues”, or “degraders” if they induce TPD. Agents that can engage surfaces, in some embodiments, novel surfaces, on various targets, including an RBR E3 ligase, RNF31, are provided in the present disclosure including in various Examples (e.g., Example 4). The present Example provides agents, e.g., stapled peptide agents such as Helicons, that bind to eight additional E3 proteins from the three remaining E3 ligase families. The present Example further describes a useful screening approach that enables the direct discovery of cooperative, molecular glue-like binders, “trimerizer” Helicons, that can lead to cooperative ternary complex formation between E3 ligases and target proteins that have been considered undruggable by small molecules. Among other things, the present Example confirm that provided technologies can be utilized to formation of various complexes including between various E3 ligases and target polypeptides.

In an example, from the first set of high-throughput screens, Applicant identified Helicons that bind to these E3s from a naive 10⁸-member phage display library of polypeptide comprising stapled peptides, e.g., Helicons (in some embodiments, 14-mer). In some embodiments, the present disclosure provides agents that bind members of the HECT family (e.g., WWP1 and WWP2). In some embodiments, the present disclosure provides agents that bind members of the Cullin-RING (CRL) multi-subunit E3 family (e.g., Cullin proteins paired with their canonical adaptor proteins (CUL1-FBXW7, CUL2-VHL, and CUL5-SOCS2)). In some embodiments, the present disclosure provides agents that bind RING/U-Box family members (e.g., MDM2 and CHIP/STUB1). In some embodiments, characterizing Helicon-E3 co-structures and possible mechanisms of action, the present disclosure identified new a-helical binding sites on the E3 surfaces, as well as potential probes of the disease-linked E3, WWP1, highlighting the generality of provided technologies against a therapeutically important target class.

In some embodiments, these findings were then used for the generation of subsequent “focused” libraries, e.g., of 20-mer Helicons, wherein the E3-binding residues were fixed and the remainder were randomized. By screening these focused libraries against target proteins in the presence and absence of the E3 presenter protein, provided technologies were able to directly identify trimerizer Helicons that cooperatively bind the targets only in the presence of the E3 “presenter” protein. In some embodiments, the present disclosure provides trimerizers that induce interactions between the E3 ubiquitin ligase CHIP and the peptidyl-prolyl cis-trans isomerase Cyclophilin A (PPIA). In some embodiments, the present disclosure provides trimerizers that induce interactions between CHIP and the transcription factor TEAD. In some embodiments, the present disclosure provides trimerizers that induce interactions between the E3 ubiquitin ligase MDM2 and the transcriptional coactivator β-catenin. Biochemical and biophysical assessment of various trimerizers confirmed their cooperative binding and their ability to inhibit protein-protein interactions. In some embodiments, x-ray co-crystal structures of two trimerizers between MDM2 and β-catenin revealed the structural basis for the interactions both between the Helicon and each protein and between the two proteins themselves. Among other things, provided technologies are useful for developing agents that induce the interaction of polypeptides, e.g., proteins, that are challenging to engage with small molecules.

### Stapled Peptide Agents Targeting Various E3 Ligases

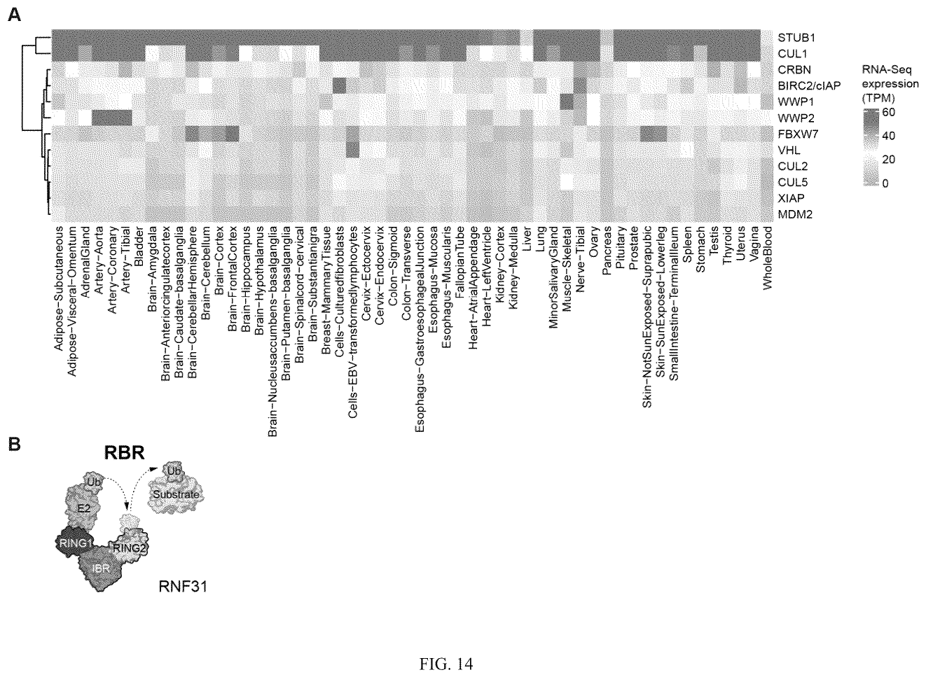

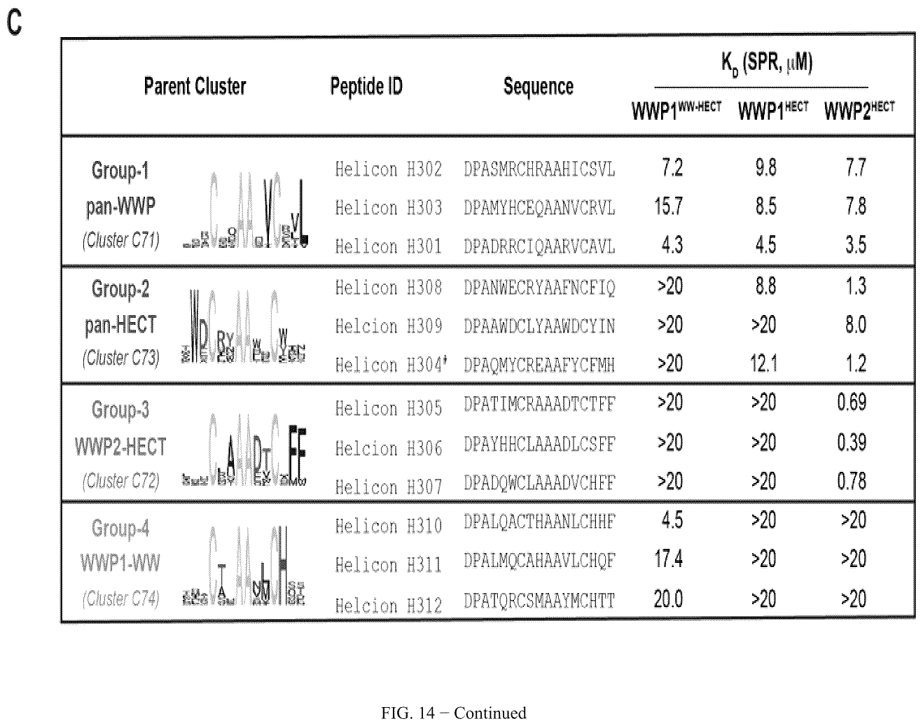

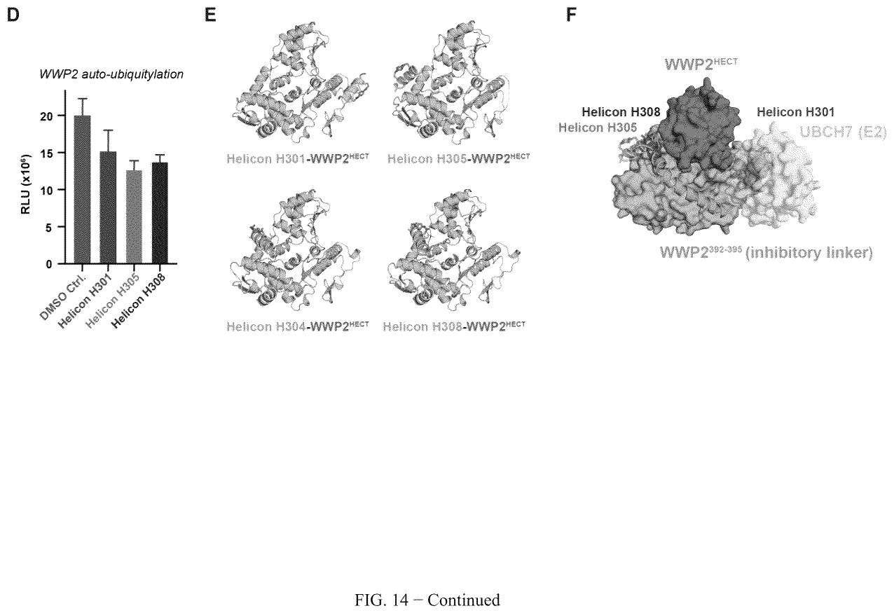

Of the estimated 600 human E3s, only a select few have been reported to be successfully targeted by small-molecule molecular glue-like molecules, e.g., molecules that co-opt the activity of Von Hippel-Lindau (VHL) and CRBN, which are members of the multi-subunit Cullin-RING (CRL) family, and of MDM2 and IAP, which are members of the single-subunit RING-finger/U-Box family. In some embodiments, the present disclosure provides agents targeting E3 ligases with a range of substrate recruitment capabilities, substrate specificities, and tissue distribution profiles. In some embodiments, the present disclosure provides agents that target members of each of the four major E3 families. Certain technologies useful for developing stapled peptide agents, e.g., for screening for Helicons that target members of each of the four major E3 families, are described herein as examples. See, e.g. FIG. 14. In some embodiments, the present disclosure provides technologies (e.g. stapled peptide agents, methods, etc.) for targeting HECT family of E3 ubiquitin ligases. In some embodiments, the present disclosure provides technologies for targeting CRL family of E3 ubiquitin ligases. In some embodiments, the present disclosure provides technologies for targeting RING/U-Box E3 family of ubiquitin ligases. In some embodiments, the present disclosure provides technologies targeting STUB1. In some embodiments, the present disclosure provide technologies for targeting CUL1. In some embodiments, the present disclosure provides technologies for targeting CRBN. In some embodiments, the present disclosure provides technologies for targeting BIRC2/cIAP. In some embodiments, the present disclosure provides technologies for targeting WWP1. In some embodiments, the present disclosure provides technologies for targeting WWP2. In some embodiments, the present disclosure provides technologies for targeting FBXW7. In some embodiments, the present disclosure provides technologies for targeting VHL. In some embodiments, the present disclosure provides technologies for targeting CUL2. In some embodiments, the present disclosure provides technologies for targeting CUL5. In some embodiments, the present disclosure provides technologies for targeting XIAP. In some embodiments, the present disclosure provides technologies for targeting MDM2. Tissue expression of various polypeptides including E3 ligases have been reported. For example, in some embodiments, expression of various polypeptides including E3 ligase can be obtained using RNA-Seq data from the GTEx Portal (gtexportal.org).

Stapled Peptide Agents that Bind the HECT E3 Family

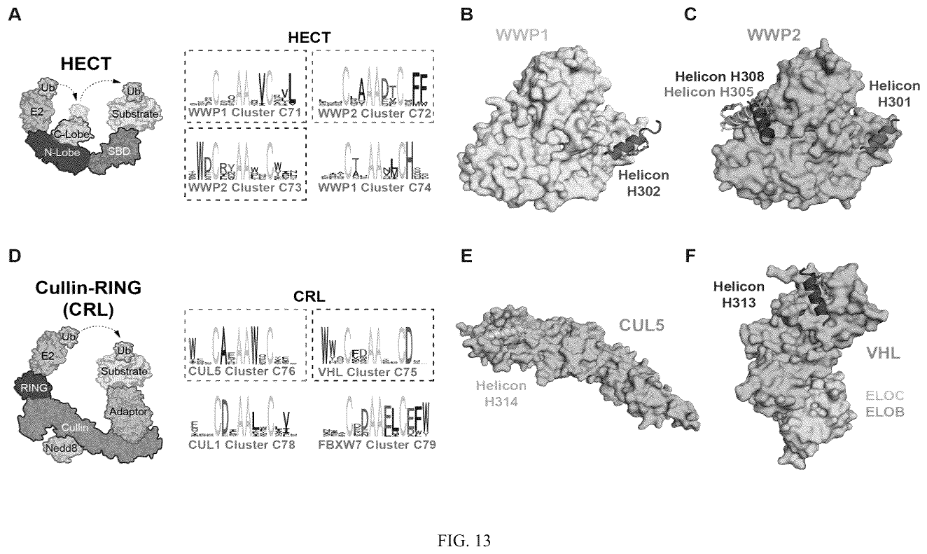

In some embodiments, the present disclosure provides technologies targeting NEDD4-like E3 ligases. It is reported that such E3 ligases can make up approximately a third of the HECT family. Such ligases include WWP1 and WWP2, which according to some reports each comprise four tandem WW domains and a catalytic HECT domain at the C-terminus that in some embodiments transfers ubiquitin from a bound E2 first to itself and subsequently to the target substrate (see, e.g., FIG. 13). It is reported that WWP1 induces the ubiquitination of several tumor suppressor proteins including p53 and PTEN, and its increased expression in some human cancers promotes cell proliferation, migration and invasion, making it an attractive therapeutic target. For instance, it has been reported that inhibition of the MYC-WWP1 axis involved in PTEN regulation using the natural product indole-3-carbinol can suppress tumors by reactivating PTEN.

In some embodiments, the present disclosure provides HECT-containing protein constructs that either stabilize the autoinhibited, inactive state of WWP1 (WWP1^{WW-HECT}). In some embodiments, the present disclosure provides WWP1 constructs that are able to adopt both the active and inactive conformations (WWP1^{HECT}). In some embodiments, the present disclosure provides WWP2 constructs that are able to adopt both the active and inactive conformations (WWP2^{HECT}). The WWP1^{WW-HECT} construct that locks WWP1 in an inactive state comprises WW domains 2-4, an inhibitory linker region that connects WW domains 2 and 3, and the HECT domain. In some embodiments, these three recombinant proteins were screened in vitro in parallel against a single naive phage library that displays ˜10⁸ 14-mer Helicons and hierarchical statistical clustering was utilized to group certain binders into families of related sequences, represented as pharmacophore logos (see, e.g., FIG. 13, (A), and FIG. 14, (C))

Several binding profiles were illustrated in the present Example (see, e.g., FIG. 14, (C), Table E11-1), with four of them described below. As demonstrated herein, a first profile (Group 1) represents certain Helicons such as those in cluster C71 (C71) that can be pan-WWP-binders—binding indiscriminately to inactive and active forms of both HECT domains, which share ˜70% sequence identity. In some embodiments, the present disclosure provides agents that can bind inactive and active forms of both HECT domains. In some embodiments, the present disclosure provides agents that can bind WWP1^{WW-HECT}, WWP1^{HECT} and WWP2^{HECT}. As demonstrated herein, a Group 2 Helicons, the pan-HECT group including C71 and C73, can bind both HECT domains. In some embodiments, the present disclosure provides agents that can bind active forms of both HECT domains. In some embodiments, the present disclosure provides agents that can bind active forms of both HECT domains selectively over inactive HECT. In some embodiments, the present disclosure provides agents that can bind WWP1^{HECT} and WWP2^{HECT}. In some embodiments, the present disclosure provides agents that can bind WWP1^{HECT} and WWP2^{HECT} selectively over WWP1^{WW-HECT}. As demonstrated herein, a Group 3 Helicons, the WWP2-HECT group including C72, can bind selectively WWP2 HECT domain (WWP2^{HECT}) In some embodiments, the present disclosure provides agents that can bind WWP2 HECT domain. In some embodiments, the present disclosure provides agents that can bind WWP2^{HECT}. In some embodiments, the present disclosure provides agents that can bind WWP2^{HECT} selectively over WWP1^{HECT} and WWP1^{WW-HECT}. As demonstrated hereon, a Group 4 Helicons, represented by C74, can bind to WWP1^{WW-HECT} but not WWP1^{HECT}. In some embodiments, provided technologies, e.g., the Group 4 Helicons, can bind inactive WWP1 HECT domain. In some embodiments, the present disclosure provides agents that can bind WWP1^{WW-HECT}. In some embodiments, the present disclosure provides agents that can bind WWP1^{WW-HECT} selectively over WWP1^{HECT} and WWP2^{HECT}. In some embodiments, an inactive WWP1 HECT domain is or comprises WWP1^{WW-HECT} or a characteristic portion thereof. In some embodiments, an active WWP1 HECT domain is or comprises WWP1^{HECT} or a characteristic portion thereof. In some embodiments, an active WWP2 HECT domain is or comprises WWP2^{HECT} or a characteristic portion thereof.

TABLE E11-1
Certain agents.

| | | | | SEQ | | | |
|---|---|---|---|---|---|---|---|
| | Helicon | Cluster | | ID | | Expected | Observed |
| Target | Name | Name | Sequence | NO: | Modification | Mass | Mass |
| WWP1/WWP2 | H301 | C71 | Ac-DPADRRCIQAARVCAVL-NH2 | 468 | Cys-stapled | 2086.395 | 2086.4 |
| WWP1/WWP2 | H302 | C71 | Ac-DPASMRCHRAAHICSVL-NH2 | 469 | Cys-stapled | 2096.413 | 2094.9 |
| WWP1/WWP2 | H303 | C71 | Ac-DPAMYHCEQAANVCRVL-NH2 | 470 | Cys-stapled | 2149.426 | 2148.0 |
| WWP1/WWP2 | H304 | C71 | Ac-DPAQMYCREAAFYCFMH-NH2 | 471 | Cys-stapled | 2310.911 | 2312.2 |
| WWP1/WWP2 | H305 | C72 | Ac-DPATIMCRAAADTCTFF-NH2 | 472 | Cys-stapled | 2063.331 | 2062.6 |
| WWP1/WWP2 | H306 | C72 | Ac-DPAYHHCLAAADLCSFF-NH2 | 473 | Cys-stapled | 2110.324 | 2109.8 |
| WWP1/WWP2 | H307 | C72 | Ac-DPADQWCLAAADVCHFF-NH2 | 474 | Cys-stapled | 2138.334 | 2138.2 |
| WWP1/WWP2 | H308 | C73 | Ac-DPANWECRYAAFNCFIQ-NH2 | 475 | Cys-stapled | 2277.489 | 2277.5 |
| WWP1/WWP2 | H309 | C73 | Ac-DPAAWDCLYAAWDCYIN-NH2 | 476 | Cys-stapled | 2219.404 | 2218.6 |
| WWP1/WWP2 | H310 | C74 | Ac-DPALQACTHAANLCHHF-NH2 | 477 | Cys-stapled | 2078.288 | 2077.2 |
| WWP1/WWP2 | H311 | C74 | Ac-DPALMQCAHAAVLCHQF-NH2 | 478 | Cys-stapled | 2084.398 | 2084.1 |
| WWP1/WWP2 | H312 | C74 | Ac-DPATQRCSMAAYMCHTT-NH2 | 479 | Cys-stapled | 2116.375 | 2116.2 |
| VHL | H313 | C75 | Ac-DPAWWNCFSAAQQCDAM-NH2 | 480 | Cys-stapled | 2173.360 | 2172.6 |
| CUL5 | H314 | C76 | Ac-DPAWYDCADAAWICTFQ-NH2 | 481 | Cys-stapled | 2205.377 | 2206.8 |
| CUL4B | H316 | C77 | Ac-DPADRWCELAAWTCDTF-NH2 | 482 | Cys-stapled | 2229.400 | 2228.2 |
| CHIP | H317 | C80 | Ac-CWEAWLLCET-NH2 | 483 | Cys-stapled | 1482.677 | 1482.7 |
| CHIP | H318 | C80 | Ac-PCYEAWVLCEY-NH2 | 484 | Cys-stapled | 1604.798 | 1604.8 |
| MDM2 | H319 | C81 | Ac-DPANHACFQAAWDCQFF-NH2 | 485 | Cys-stapled | 2200.364 | 2200.4 |
| MDM2 | P320 | | Ac-LTF-R8-EYWAQ-Cba- | 486 | R8-S5 | 1745.020 | 1745.3 |
| | | | S5-SAA-NH2 | | stapled | | |
| CHIP-TEAD4 | H321 | C87 | Ac-PVPFFWECQYAAATCQTPRIK-NH2 | 487 | Cys-stapled | 2686.066 | 2685.5 |
| CHIP-TEAD4 | H322 | C87 | Ac-PVPFFWECQYAAATCQ-NH2 | 488 | Cys-stapled | 2090.333 | 2089.4 |
| CHIP-TEAD4 | H323 | C87 | Ac-PTPFFWECQFAAATCTAPRVQ-NH2 | 489 | Cys-stapled | 2600.919 | 2600.8 |
| CHIP-TEAD4 | H324 | C87 | Ac-PVPFFWDCQFAAATCDAPQRR-NH2 | 490 | Cys-stapled | 2655.957 | 2655.9 |
| CHIP-TEAD4 | P325 | | Ac-LWWPDGSGSGGSPGQVPMRKRQLPA | 491 | unstapled | 3638.026 | 3637.5 |
| | | | SFWEEPR-NH2 | | | | |
| CHIP-PPIA | H326 | C88 | Ac-PAQDDWSCVEAAYLCENYVRV-NH2 | 492 | Cys-stapled | 2660.883 | 2661.0 |
| CHIP-PPIA | H327 | C89 | Ac-PILQGMACGPAATICWIDGII-NH2 | 493 | Cys-stapled | 2372.819 | 2372.8 |
| CHIP-PPIA | H328 | C89 | Ac-PDMLAPMCGPAASICWIDGVI-NH2 | 494 | Cys-stapled | 2389.827 | 2389.8 |
| MDM2-β-catenin | H329 | C91 | Ac-PMEQQAICFQAAWMCLADDWT-NH2 | 27 | Cys-stapled | 2688.037 | 2688.4 |
| MDM2-β-catenin | H330 | C91 | Ac-PWKYEQVCYQAAWQCLSDDWD- | 28 | Cys-stapled | 2864.078 | 2863.7 |
| | | | NH2 | | | | |
| MDM2-β-catenin | H331 | C91 | Ac-QAICFQAAWMCLADDWT-NH2 | 495 | Cys-stapled | 2202.484 | 2201.6 |
| MDM2-β-catenin | H332 | C92 | Ac-PISAANDCFKAAWQCIIWLHQ-NH2 | 496 | Cys-stapled | 2645.017 | 2645.1 |
| MDM2-β-catenin | H333 | C92 | Ac-PSENARDCFWAAWDCLYFIYQ-NH2 | 497 | Cys-stapled | 2828.091 | 2827.6 |
| MDM2-β-catenin | H334 | C93 | Ac-PNLTGADCFPAAWQCLQFLWD-NH2 | 498 | Cys-stapled | 2625.925 | 2625.8 |
| MDM2-β-catenin | P335 | | Ac-PD-cyclopentylalanine- | 43 | Cys-stapled | 3760.075 | 3758.7 |
| | | | CDDAAFNC-3Thi- | | | | |
| | | | benzothienylalanine- | | | | |
| | | | QGSGS-bAla- | | | | |
| | | | LTFEHYWAQLTS-NH2 | | | | |

Selected Helicons from representative clusters from each group were synthesized, crosslinked, and biochemically validated. Most of the phage hits bound the proteins with μM affinity, as assessed by surface plasmon resonance (SPR). See, e.g., FIG. 14, (C) and data below in Table E11-2. The most potent hit from the single-round screen, H306, had a binding affinity (K_{D}) of 390 nM, against WWP2^{HECT}. We found that individual Helicons from all three HECT-binding Groups (1-3) could modestly inhibit the auto-ubiquitylation activity of the isolated WWP2^{HECT} (see, e.g. FIG. 14, (D)).

TABLE E11-2
Certain agents and interaction data.
WWP1-C71

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAQQTCSQAAWVCSIL | 500 | 0 | 55 | 443 | 363 | 397 | 477 | 0 | 0 |
| DPASMRCHRAAHICSVL | 501 | 0 | 21 | 288 | 218 | 244 | 281 | 0 | 0 |
| DPAIQRCSTAAQVCRYL | 502 | 0 | 6 | 202 | 236 | 306 | 197 | 0 | 0 |
| DPAMYHCEQAANVCRVL | 503 | 32 | 86 | 259 | 381 | 306 | 337 | 0 | 1 |
| DPAATLCNQAANVCALL | 504 | 0 | 0 | 132 | 145 | 153 | 197 | 0 | 0 |
| DPARLDCTAAAYVCSVL | 505 | 0 | 0 | 109 | 91 | 153 | 140 | 0 | 0 |
| DPAIMSCMYAAEVCRVL | 506 | 0 | 1 | 147 | 109 | 183 | 253 | 0 | 0 |
| DPALQTCMDAAQVCRLL | 507 | 0 | 0 | 107 | 163 | 153 | 197 | 0 | 0 |
| DPAVRDCSDAAIVCELV | 508 | 5 | 5 | 147 | 91 | 306 | 197 | 0 | 0 |
| DPALASCQQAAWVCEVL | 509 | 0 | 43 | 173 | 291 | 306 | 309 | 0 | 0 |
| DPAMRRCHQAAWVCEVL | 510 | 0 | 1 | 98 | 182 | 183 | 140 | 0 | 0 |
| DPAHTACNQAARVCHTL | 511 | 1 | 4 | 104 | 200 | 153 | 253 | 0 | 0 |
| DPADRRCIQAARVCAVL | 512 | 30 | 30 | 95 | 73 | 153 | 225 | 0 | 0 |
| DPAIFDCEFAAHVCSVL | 513 | 0 | 26 | 222 | 436 | 275 | 618 | 0 | 3 |
| DPARWACTEAAHVCDTL | 514 | 0 | 2 | 112 | 182 | 183 | 281 | 0 | 0 |

WWP2-C72

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPARVDCLVAADMCHMF | 515 | 165 | 532 | 522 | 555 | 466 | 573 | 0 | 0 |
| DPATIMCRAAADTCTFF | 516 | 38 | 83 | 92 | 106 | 86 | 79 | 0 | 0 |
| DPAYHHCLAAADLCSFF | 517 | 59 | 97 | 107 | 147 | 101 | 141 | 0 | 0 |
| DPATYYCEAAAETCRFF | 518 | 37 | 57 | 70 | 94 | 78 | 123 | 0 | 0 |
| DPALWECMTAADWCMFM | 519 | 1 | 24 | 17 | 18 | 28 | 32 | 0 | 0 |
| DPADQWCLAAADVCHFF | 520 | 112 | 222 | 322 | 349 | 344 | 392 | 0 | 0 |
| DPARVNCHAAADTCFFF | 521 | 6 | 83 | 76 | 103 | 112 | 123 | 0 | 0 |
| DPALWHCLAAAETCAMF | 522 | 4 | 38 | 24 | 49 | 47 | 76 | 0 | 0 |
| DPAIHSCYAAAETCQFF | 523 | 43 | 87 | 89 | 139 | 127 | 194 | 0 | 0 |
| DPAANYCEAAADTCQFF | 524 | 0 | 179 | 254 | 297 | 283 | 378 | 0 | 0 |
| DPALLHCMAAAQTCSFF | 525 | 0 | 47 | 26 | 37 | 59 | 123 | 0 | 0 |
| DPAQDTCRAAADTCAFF | 526 | 8 | 159 | 267 | 241 | 232 | 373 | 0 | 0 |
| DPAQNMCQAAADVCMFF | 527 | 2 | 67 | 51 | 125 | 120 | 127 | 0 | 0 |
| DPALIHCRAAADTCAFW | 528 | 87 | 318 | 384 | 608 | 419 | 565 | 0 | 0 |
| DPAIQNCRSAADVCMFF | 529 | 0 | 65 | 96 | 109 | 117 | 127 | 0 | 0 |

WWP2-C73

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPANWECRYAAFNCFIQ | 530 | 0 | 40 | 41 | 40 | 55 | 86 | 0 | 0 |
| DPAVWECRWAAFQCWLD | 531 | 0 | 0 | 88 | 86 | 97 | 107 | 0 | 0 |
| DPAAWDCLYAAWDCYIN | 532 | 1 | 17 | 382 | 567 | 427 | 516 | 0 | 0 |
| DPATWDCNVAAHRCWFL | 533 | 1 | 0 | 12 | 60 | 40 | 75 | 0 | 0 |
| DPAVWDCRYAAWDCYTN | 534 | 0 | 0 | 0 | 28 | 33 | 15 | 0 | 0 |
| DPATWDCRNAAWLCLLN | 535 | 0 | 1 | 4 | 8 | 12 | 12 | 0 | 0 |
| DPALWECRNAAWQCWMH | 536 | 0 | 0 | 0 | 24 | 35 | 27 | 0 | 0 |
| DPAMWDCRYAALDCYMT | 537 | 0 | 0 | 6 | 23 | 62 | 24 | 0 | 0 |
| DPANWDCQQAAWLCIIQ | 538 | 0 | 0 | 0 | 8 | 7 | 27 | 0 | 0 |
| DPAMWSCRYAAWECWVL | 539 | 0 | 0 | 0 | 8 | 7 | 28 | 0 | 0 |
| DPAIWDCLYAATNCYIN | 540 | 0 | 0 | 0 | 8 | 12 | 20 | 0 | 0 |
| DPATWDCQQAAHHCWVL | 541 | 0 | 0 | 2 | 23 | 35 | 66 | 1 | 0 |
| DPATWDCIWAAERCWRY | 542 | 0 | 0 | 1 | 4 | 9 | 13 | 0 | 0 |
| DPATWACINAAHNCWRN | 543 | 0 | 0 | 0 | 6 | 23 | 15 | 0 | 0 |
| DPAAWDCRYAALACYIH | 544 | 0 | | 2 | 11 | 66 | 34 | 0 | 0 |

WWP1-C74

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPALQACTHAANLCHHF | 545 | 0 | 11 | 40 | 218 | 244 | 169 | 0 | 0 |
| DPATHVCTTAA VMCHQT | 546 | 0 | 0 | 6 | 327 | 275 | 365 | 2 | 0 |
| DPATMNCTLAANICHSH | 547 | 0 | 0 | 3 | 363 | 458 | 197 | 0 | 0 |
| DPAFLTCTLAASLCHQD | 548 | 0 | 0 | 0 | 145 | 153 | 253 | 0 | 0 |
| DPATETCAQAAVLCHSD | 549 | 1 | 12 | 3 | 200 | 519 | 449 | 0 | 0 |
| DPAWMACAWAANLCHSS | 550 | 0 | 18 | 14 | 91 | 244 | 253 | 0 | 0 |
| DPAIIRCTSAAMMCHNT | 551 | 0 | 4 | 0 | 55 | 183 | 169 | 0 | 0 |
| DPAHQTCAFAAVLCHQN | 552 | 0 | 0 | 32 | 18 | 153 | 309 | 0 | 0 |
| DPAMIACSHAAQMCHWS | 553 | 0 | 8 | 3 | 454 | 275 | 281 | 0 | 1 |
| DPAFIQCTHAANMCHSS | 554 | 0 | 3 | 43 | 381 | 336 | 562 | 0 | 0 |
| DPAWVSCVRAANLCHVS | 555 | 33 | 1 | 0 | 0 | 183 | 140 | 0 | 0 |
| DPALMQCAHAAVLCHQF | 556 | 0 | 2 | 0 | 400 | 550 | 281 | 8 | 0 |
| DPATQRCSMAAYMCHTT | 557 | 1 | 47 | 155 | 509 | 672 | 1067 | 40 | 0 |
| DPAQLSCTVAAHVCHST | 558 | 0 | 25 | 0 | 254 | 275 | 140 | 9 | 0 |
| DPAFHYCTMAANMCHQD | 559 | 0 | 0 | 3 | 73 | 153 | 169 | 0 | 4 |

VHL-C75

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAWWHCVAAATQCDQR | 560 | 0 | 20 | 56 | 79 | 24 | 0 | 0 | |
| DPAWYACVDAAYFCDYQ | 561 | 0 | 5 | 34 | 35 | 31 | 0 | 0 | |
| DPAWWHCVDAALQCDYY | 562 | 0 | 10 | 59 | 40 | 104 | 0 | 0 | |
| DPAWWDCFNAARRCDDI | 563 | 0 | 1 | 19 | 43 | 6 | 0 | 0 | |
| DPAWWQCFDAAAYCDVH | 564 | 0 | 1 | 24 | 7 | 77 | 0 | 0 | |
| DPAWWYCMDAATMCDQQ | 565 | 0 | 1 | 39 | 19 | 51 | 0 | 0 | |
| DPAWWYCLDAAYDCDHT | 566 | 2 | 2 | 45 | 97 | 23 | 0 | 0 | |
| DPAWWNCIDAAILCDDY | 567 | 0 | 1 | 14 | 18 | 16 | 0 | 0 | |
| DPAWYACYDAARLCDNF | 568 | 0 | 3 | 43 | 78 | 46 | 0 | 0 | |
| DPAWWNCFSAAQQCDAM | 569 | 8 | 16 | 24 | 27 | 65 | 0 | 0 | |
| DPAWYACMDAARVCDRT | 570 | 8 | 2 | 30 | 158 | 23 | 0 | 0 | |
| DPAWHQCFRAAYMCDNH | 571 | 0 | 2 | 9 | 57 | 7 | 0 | 0 | |
| DPAWHACFQAAYICDQA | 572 | 0 | 3 | 11 | 31 | 18 | 0 | 0 | |
| DPAWWSCFAAAHHCDAL | 573 | 0 | 0 | 20 | 73 | 35 | 0 | 0 | |
| DPAWWDCFNAAMRCDIS | 574 | 0 | 0 | 10 | 59 | 12 | 0 | 0 | |

CUL5-C76

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAWRECAHAAWQCYIQ | 75 | 0 | 50 | 47 | 21 | 93 | 0 | 0 | |
| DPAWYDCADAAWICTFQ | 576 | 7 | 193 | 188 | 231 | 389 | 0 | 0 | |
| DPAWAICAEAAWDCFMI | 577 | 0 | 14 | 13 | 58 | 11 | 0 | 0 | |
| DPAYYVCAEAAWNCLFE | 578 | 4 | 46 | 104 | 100 | 76 | 0 | 0 | |
| DPAIQQCAEAAWSCYVE | 579 | 0 | 13 | 30 | 40 | 14 | 0 | 0 | |
| DPAWHMCAEAAWQCAFW | 580 | 5 | 61 | 106 | 119 | 152 | 0 | 0 | |
| DPAWFTCAQAAWDCVFH | 581 | 0 | 65 | 112 | 96 | 224 | 0 | 0 | |
| DPAWFDCAEAAWQCVFI | 582 | 2 | 35 | 91 | 170 | 31 | 0 | 0 | |
| DPAWRACAQAAWDCIIA | 583 | 1 | 167 | 267 | 385 | 368 | 0 | 0 | |
| DPAWQWCAEAAWQCITH | 584 | 14 | 59 | 284 | 284 | 265 | 0 | 0 | |
| DPATFACANAAWECYAR | 585 | 0 | 6 | 25 | 11 | 35 | 0 | 0 | |
| DPAWMRCATAAWECVMT | 586 | 16 | 22 | 151 | 104 | 222 | 0 | 0 | |
| DPAWWECAEAAWQCQVL | 587 | 0 | 6 | 44 | 20 | 59 | 0 | 0 | |
| DPAWMTCATAAWECYLA | 588 | 6 | 6 | 62 | 48 | 55 | 0 | 0 | |
| DPAWYECASAAWVCYSM | 589 | 0 | 13 | 94 | 60 | 171 | 0 | 0 | |

CUL4B-C77

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPADRWCELAAWTCDTF | 590 | 1 | 25 | 191 | 321 | 234 | 0 | 1 | |
| DPATTECILAAWICDRF | 591 | 0 | 0 | 15 | 31 | 18 | 0 | 0 | |
| DPASRLCELAAWVCDSF | 592 | 4 | 15 | 139 | 319 | 251 | 0 | 0 | |
| DPASRMCEMAAWFCENY | 593 | 0 | 3 | 11 | 13 | 78 | 0 | 0 | |
| DPAMNHCVLAAWVCDTF | 594 | 0 | 0 | 6 | 37 | 9 | 0 | 0 | |
| DPAQRACQFAAWICERF | 595 | 1 | 0 | 13 | 56 | 73 | 0 | 0 | |
| DPADRQCIFAAWMCEAF | 596 | 0 | 0 | 3 | 9 | 34 | 0 | 0 | |
| DPADRYCDMAAWVCERF | 597 | 1 | 1 | 15 | 248 | 340 | 0 | 0 | |
| DPAERHCQLAAWVCHQF | 598 | 0 | 0 | 0 | 99 | 29 | 0 | 0 | |
| DPADRLCELAAWVCQSF | 599 | 0 | 0 | 0 | 48 | 9 | 0 | 0 | |
| DPADRWCELAAWICDEF | 600 | 0 | 0 | 0 | 6 | 17 | 0 | 0 | |
| DPATRHCIFAAWACDRF | 601 | 0 | 0 | 47 | 20 | 12 | 0 | 0 | |
| DPATRMCELAAWTCEVW | 602 | 0 | 0 | 1 | 31 | 9 | 0 | 0 | |
| DPADRQCLFAAWYCEHT | 603 | 0 | 10 | 0 | 6 | 11 | 2 | 0 | |

CUL1-C78

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAYDWCDVAALFCLVE | 604 | 0 | 0 | 57 | 36 | 57 | 151 | 0 | 0 |
| DPAYFACDIAALMCTVH | 605 | 0 | 2 | 131 | 135 | 403 | 120 | 0 | 0 |
| DPAMLQCDMAALWCMVS | 606 | 1 | 3 | 77 | 64 | 143 | 126 | 0 | 0 |
| DPAQILCDLAALYCVVE | 607 | 0 | 7 | 61 | 73 | 111 | 119 | 0 | 0 |
| DPAQNDCEVAAFFCWLD | 608 | 1 | 1 | 233 | 428 | 586 | 468 | 0 | 0 |
| DPAEQECDHAALWCIVA | 609 | 14 | 19 | 340 | 937 | 1137 | 743 | 0 | 0 |
| DPAEFVCDWAALACLVD | 610 | 4 | 32 | 183 | 247 | 517 | 300 | 0 | 0 |
| DPAEMQCDYAAILCIVR | 611 | 0 | 0 | 32 | 95 | 114 | 71 | 0 | 0 |
| DPAIIQCDQAALWCVVT | 612 | 0 | 0 | 28 | 83 | 80 | 61 | 0 | 0 |
| DPAEAACDIAALMCIID | 613 | 15 | 13 | 65 | 211 | 203 | 195 | 0 | 0 |
| DPAVMECDWAATICDIF | 614 | 0 | 2 | 154 | 402 | 477 | 419 | 0 | 0 |
| DPAEVHCDMAALYCIIR | 615 | 3 | 7 | 65 | 242 | 366 | 119 | 0 | 0 |
| DPAEQMCDIAAVWCLAH | 616 | 0 | 0 | 35 | 135 | 151 | 88 | 0 | 0 |

FBXW7-C79

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPADLTCEDAAELCEFW | 617 | 2 | 80 | 352 | 535 | 1005 | 510 | 0 | 0 |
| DPAFWYCDEAAEMCEFW | 618 | 0 | 0 | 20 | 28 | 91 | 17 | 0 | 0 |
| DPANIACDDAALLCAFW | 619 | 0 | 0 | 2 | 7 | 5 | 8 | 0 | 0 |
| DPANIACDDAALLCELW | 620 | 0 | 1 | 1 | 7 | 8 | 6 | 0 | 0 |
| DPANIACDDAALLCEFW | 621 | 4 | 131 | 203 | 946 | 1140 | 1464 | 0 | 0 |
| DPADMYCRDAAELCEFW | 622 | 1 | 23 | 187 | 302 | 505 | 554 | 0 | 0 |
| DPAAFFCDDAAERCEFW | 623 | 4 | 39 | 35 | 163 | 245 | 449 | 0 | 0 |
| DPATYFCYDAAELCDWW | 624 | 0 | 0 | 1 | 5 | 5 | 82 | 0 | 0 |
| DPAITTCLDAAELCDFW | 625 | 1 | 26 | 61 | 34 | 215 | 132 | 0 | 0 |
| DPAAWICEEAAEICEFW | 626 | 0 | 0 | 5 | 4 | 15 | 36 | 0 | 0 |
| DPAYFSCEDAAELCEWY | 627 | 0 | 0 | 1 | 3 | 13 | 38 | 0 | 0 |
| DPADMMCENAALLCEFW | 628 | 1 | 3 | 68 | 20 | 282 | 228 | 0 | 0 |
| DPAMTWCLNAAELCEFW | 629 | 0 | 0 | 0 | 3 | 17 | 83 | 0 | 0 |
| DPAIRMCRDAAELCEFW | 630 | 0 | 0 | 1 | 4 | 66 | 94 | 0 | 0 |
| DPANWVCMDAAELCDFY | 631 | 0 | 0 | 5 | 2 | 99 | 15 | 0 | 0 |

CHIP-C80

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| PCWDAWMLCTD | 632 | 19 | 142 | 542 | 632 | 701 | 581 | 0 | 0 |
| ICYNARLECE | 633 | 1 | 2 | 13 | 16 | 21 | 20 | 0 | 0 |
| ICYTAWLECE | 634 | 0 | 1 | 5 | 9 | 7 | 12 | 0 | 0 |
| ICYSAWLECE | 635 | 1 | 3 | 13 | 25 | 22 | 28 | 0 | 0 |
| PCYSAWITCES | 636 | 0 | 134 | 286 | 457 | 563 | 538 | 0 | 0 |
| ICYNAWLECA | 637 | 0 | 1 | 5 | 7 | 11 | 9 | 0 | 0 |
| ICYNAWFECE | 638 | 0 | 2 | 9 | 11 | 18 | 14 | 0 | 0 |
| PCWNAWMVCSV | 639 | 2 | 44 | 334 | 474 | 690 | 691 | 0 | 0 |
| ICYNAWVECE | 640 | 1 | 2 | 10 | 21 | 19 | 23 | 0 | 0 |
| PCFEAWVMCEI | 641 | 4 | 83 | 121 | 285 | 375 | 297 | 0 | 0 |
| PCYSAWIVCSD | 642 | 4 | 81 | 123 | 444 | 549 | 468 | 0 | 0 |
| PCWNAWLYCSN | 643 | 0 | 0 | 11 | 105 | 155 | 58 | 0 | 0 |
| ICWAAWVSCS | 644 | 0 | 0 | 0 | 25 | 42 | 17 | 0 | 0 |
| PCWSAWLACEL | 645 | 0 | 81 | 72 | 130 | 220 | 206 | 0 | 0 |
| PMCSDAWLFCE | 646 | 0 | 0 | 0 | 74 | 143 | 57 | 0 | 0 |
| PCWEAWLWCSM | 647 | 0 | 0 | 4 | 124 | 194 | 145 | 0 | 0 |

MDM2-C81

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAYASCFSAAWDCQFW | 648 | 0 | 0 | 19 | 21 | 13 | 38 | 0 | 0 |
| DPAFDSCFSAAWDCMYW | 649 | 3 | 0 | 27 | 92 | 65 | 84 | 0 | 0 |
| DPAENECFTAAWDCMFF | 650 | 0 | 0 | 7 | 51 | 70 | 40 | 0 | 0 |
| DPANHACFQAAWDCQFF | 651 | 11 | 154 | 643 | 1045 | 776 | 1088 | 1 | 0 |
| DPASEACFQAAWDCVSW | 652 | 2 | 57 | 241 | 559 | 336 | 447 | 1 | 0 |
| DPATQQCFQAAWDCQFM | 653 | 1 | 60 | 324 | 683 | 382 | 679 | 8 | 0 |
| DPADNRCFQAAWDCAFW | 654 | 1 | 0 | 51 | 22 | 20 | 23 | 0 | 0 |
| DPADSECFQAASDCQYW | 655 | 7 | 11 | 0 | 0 | 5 | 6 | 1 | 0 |

MDM2-C82

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAINTCQDAAFWCYWS | 656 | 0 | 131 | 203 | 226 | 185 | 315 | 0 | 0 |
| DPAETDCSQAAFWCYWQ | 657 | 1 | 15 | 170 | 133 | 176 | 188 | 0 | 0 |
| DPAHNLCFDAAWWCHWQ | 658 | 0 | 0 | 9 | 7 | 8 | 11 | 0 | 0 |
| DPARVHCNQAAFMCHWV | 659 | 0 | 0 | 9 | 6 | 14 | 8 | 0 | 0 |
| DPALMQCTEAAFWCYWE | 660 | 0 | 0 | 24 | 36 | 24 | 32 | 0 | 0 |
| DPALFSCDSAAFSCYWY | 661 | 0 | 0 | 25 | 17 | 25 | 57 | 0 | 0 |
| DPATESCEDAAFWCHWI | 662 | 0 | 0 | 30 | 30 | 30 | 70 | 0 | 0 |
| DPAYTQCSDAAFMCWWW | 663 | 0 | 19 | 32 | 48 | 65 | 62 | 0 | 0 |
| DPAQHHCQQAAFWCQWW | 664 | 0 | 0 | 13 | 13 | 36 | 18 | 0 | 0 |
| DPARLRCQDAAFACMWY | 665 | 0 | 0 | 17 | 31 | 32 | 37 | 0 | 0 |
| DPARRECMDAAFWCQWV | 666 | 0 | 53 | 106 | 232 | 148 | 261 | 0 | 0 |
| DPAHYECTDAAFECYWF | 667 | 0 | 2 | 33 | 74 | 57 | 82 | 0 | 0 |
| DPAQEDCHAAAFWCIWD | 668 | 0 | 0 | 15 | 37 | 23 | 48 | 0 | 0 |
| DPADIVCQDAAFQCFWW | 669 | 0 | 1 | 12 | 33 | 46 | 18 | 0 | 0 |
| DPAAVQCNTAAFQCYWF | 670 | 0 | 0 | 23 | 56 | 53 | 69 | 0 | 0 |

CHIP-C83

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAFEVCQFAAATCSSN | 671 | 0 | 0 | 8 | 10 | 6 | 30 | 0 | 0 |
| DPAFWSCQFAAATCSHL | 672 | 0 | 0 | 4 | 6 | 11 | 8 | 0 | 0 |
| DPAMWHCQYAAIMCNAN | 673 | 0 | 0 | 0 | 15 | 7 | 29 | 0 | 0 |
| DPAFVRCQFAAASCHTI | 674 | 0 | 0 | 0 | 22 | 27 | 19 | 0 | 0 |
| DPAFEYCQWAAATCQMD | 675 | 0 | 12 | 0 | 43 | 46 | 90 | 0 | 0 |
| DPAFEQCQFAAAMCLHH | 676 | 0 | 6 | 1 | 17 | 42 | 21 | 0 | 0 |
| DPAFTACQFAAAMCDSS | 677 | 0 | 0 | 0 | 3 | 8 | 5 | 0 | 0 |
| DPAFWRCQFAAATCEHT | 678 | 2 | 3 | 210 | 321 | 597 | 726 | 1 | 0 |
| DPAFWQCQFAAATCAHY | 679 | 0 | 0 | 19 | 16 | 70 | 44 | 0 | 0 |
| DPAFERCQLAAAFCYDS | 680 | 0 | 0 | 2 | 15 | 60 | 47 | 0 | 0 |
| DPAFDACQFAAAMCDHQ | 681 | 0 | 30 | 23 | 39 | 80 | 248 | 0 | 0 |
| DPALWNCQYAAVMCTQL | 682 | 0 | 0 | 0 | 14 | 34 | 75 | 0 | 0 |
| DPAFWVCQYAAAMCQDV | 683 | 8 | 3 | 160 | 86 | 331 | 323 | 1 | 0 |
| DPAFDRCQWAAALCMMN | 684 | 0 | 0 | 4 | 13 | 31 | 76 | 0 | 0 |
| DPAFTRCQFAAAVCNFT | 685 | 0 | 3 | 0 | 6 | 19 | 28 | 0 | 0 |

CHIP-C84

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAEYACYHAASICNVD | 686 | 0 | 0 | 54 | 61 | 31 | 91 | 0 | 0 |
| DPAIIWCSQAASICTVD | 687 | 0 | 0 | 19 | 15 | 23 | 24 | 0 | 0 |
| DPAYDVCHFAAHICWID | 688 | 0 | 0 | 22 | 19 | 20 | 62 | 0 | 0 |
| DPANHICHYAAHICDVD | 689 | 0 | 0 | 3 | 15 | 6 | 49 | 0 | 0 |
| DPALEECTQAAHICMVD | 690 | 0 | 0 | 3 | 14 | 26 | 10 | 0 | 0 |
| DPAETFCWDAADICMVD | 691 | 0 | 0 | 1 | 17 | 7 | 32 | 0 | 0 |
| DPAVHNCSQAASICDVD | 692 | 0 | 0 | 0 | 22 | 9 | 39 | 0 | 0 |
| DPAVRWCAAAAAICMVD | 693 | 0 | 0 | 0 | 21 | 16 | 22 | 0 | 0 |
| DPASQWCQRAASICWID | 694 | 0 | 0 | 0 | 15 | 18 | 8 | 0 | 0 |
| DPAELFCTEAADICMVD | 695 | 0 | 0 | 0 | 18 | 13 | 22 | 0 | 0 |
| DPAQYNCYFAANICTVD | 696 | 0 | 0 | 0 | 48 | 79 | 17 | 0 | 0 |
| DPAINMCYRAAQICDVD | 697 | 0 | 0 | 0 | 31 | 28 | 27 | 0 | 0 |
| DPAFFECVRAASICMVD | 698 | 0 | 0 | 0 | 51 | 41 | 54 | 0 | 0 |
| DPAQDNCHYAAHICMVD | 699 | 0 | 0 | 0 | 47 | 25 | 57 | 0 | 0 |

MDM2-C85

| | SEQ ID | Target Concentration | | | | | | | |
|---|---|---|---|---|---|---|---|---|---|
| Sequence | NO: | 10 | 30 | 100 | 300 | 1000 | 1000 | BLANK | BLANK |
| DPAMSNCFMAAWECLIQ | 701 | 49 | 104 | 206 | 192 | 185 | 176 | 0 | 0 |
| DPAYSDCFQAAWTCMIH | 702 | 0 | 78 | 135 | 155 | 110 | 150 | 0 | 0 |
| DPASAECFFAAWQCIAT | 703 | 0 | 8 | 28 | 27 | 33 | 14 | 0 | 0 |
| DPAHRQCFTAAWRCMMT | 704 | 0 | 42 | 149 | 156 | 202 | 149 | 0 | 0 |
| DPADRDCFSAAWACVIR | 705 | 0 | 37 | 188 | 209 | 160 | 215 | 0 | 0 |
| DPASQDCFSAAWQCLQT | 706 | 0 | 31 | 138 | 165 | 94 | 231 | 0 | 0 |
| DPAVTECFQAAWRCMVM | 707 | 0 | 77 | 184 | 276 | 269 | 268 | 0 | 0 |
| DPAQQDCFLAAWDCVWQ | 708 | 0 | 12 | 132 | 166 | 104 | 237 | 0 | 0 |
| DPARQDCFSAAWMCMAI | 709 | 0 | 1 | 94 | 107 | 120 | 114 | 0 | 0 |
| DPAQSDCFQAAWECVAN | 710 | 0 | 0 | 35 | 47 | 52 | 29 | 0 | 0 |
| DPAERNCFTAAWQCMVW | 711 | 0 | 23 | 158 | 230 | 135 | 225 | 0 | 0 |
| DPAAQECFRAAWRCMQD | 712 | 0 | 0 | 25 | 37 | 34 | 29 | 0 | 0 |
| DPASADCFNAAWDCMWI | 713 | 0 | 19 | 102 | 165 | 124 | 209 | 0 | 0 |
| DPAHQQCFMAAWRCVVH | 714 | 1 | 48 | 230 | 375 | 356 | 327 | 0 | 0 |
| DPAYEDCFFAAWNCMMV | 715 | 0 | 5 | 18 | 17 | 23 | 39 | 0 | 0 |

Blank = Media only.
Unless otherwise noted, concentration in nM.

Crystal structures of four Helicons representing each of the three HECT-binding clusters in complex with WWP2^{HECT}, and one Helicon from Group 1, H302, with WWP1^{HECT} were solved. See, e.g., FIG. 13, (B) and (C), FIG. 14, (E) and Table E1 1-2). Among other things, these structures revealed three distinct binding sites on WWP2^{HECT}. Without the intention to be limited by theory, in all of the structures, both HECT domains adopted similarly closed (inhibited) conformations. In some embodiments, The WWP2 proteins in the co-structures with H301, H305, and H308 share a low root-mean-square deviation (RMSD=0.5-0.8 Å) and are shown overlaid (see, e.g., FIG. 14, (C)). Without the intention to be limited by theory, consistent with their ability to impact the catalytic activity of WWP2^{HECT}, H301 binds near the E2 interface, while H305 and H308 may restrict the conformational change involved in activating this domain. It was observed that the prominence of various individual amino acid letters in the logo correlates well with its role in direct binding to the target protein.

In some embodiments, although they belonged to different clusters, the Group 2 Helicons H304 and H308 were observed to bind WWP2^{HECT} using similar residues, interacting with N- and C-lobes of the HECT domain and overlapping the binding site of WWP2's linker domain, which is reported to be necessary for its auto-inhibition (see, e.g., FIG. 13, (C); and FIG. 14 (F)). In some embodiments, it was observed that while the WWP2 linker region was mostly helical, it was linear where it overlapped the H308-binding site. In some embodiments, it was observed that Group 3 Helicon H305 bound a similar location near the interface of the N- and C-lobes of WWP2^{HECT}, with binding residues predominantly in contact with the C-lobe.

In some embodiments, it was observed that Group 1 Helicons H302 (WWP1^{HECT}-binding) and H301 (WWP2^{HECT}-binding) showed similar binding modes, making contact only with the N-lobe of either WWP1^{HECT} (see, e.g., FIG. 13, (B)) or WWP2^{HECT} (see, e.g., FIG. 13, (C); FIG. 14, (E) and (F)). A similar binding site was reported on HECT-family E3 ligases for bicyclic peptides that acted as competitive inhibitors of the E3 catalytic activity via disrupting interactions with cognate E2 ligases. Stapled peptide agents that bind the Cullin-RING E3 family

Having confirmed that provided technologies, e.g., screening platform can successfully discover novel binding sites and Helicon binders against the HECT family of E3 ligases, Applicant further confirmed that provided technologies can be utilized against the larger Cullin-RING (CRL) family. It has been reported that, among other things, as the HECT family has been shown to be coopted by viral oncoproteins, exemplified by human papilloma virus E6 which binds to host E3 E6AP to induce degradation of the tumor suppressor p53, there are numerous reported examples of viral proteins that can recruit and rewire the substrate specificities of host CRLs to induce degradation of host immunity proteins. In some embodiments, the present disclosure provides technologies, e.g., Helicons, that can modulate CRL structures and/or functions. In some embodiments, the present disclosure provides technologies that can inhibit or promote PPIs of CRL.

According to some reports, CRL is the largest E3 family, and in some cases is unique in that its members are modular—Cullin (CUL) proteins form the central scaffold that recruits an E2 enzyme via a RING-Box protein (typically RBX1 or RBX2) to its N-terminus and a CUL-specific adaptor that bridges the C-terminus of the CUL protein to the substrate protein (see, e.g., FIG. 13, (D)). In some embodiments, in case of CRLs scaffolded by CUL1 and CUL7 (CRL1 and CRL7), the adaptor is a sub-complex called SCF comprising SKP1 and an F-box protein. In some embodiments, adaptors scaffolded by CUL2, CUL3, CUL4A/4B, and CUL5 are variable substrate recruitment subunits. According to some reports, VHL (CRL2) and CRBN (CRL4) are among the most prominent members of the CRL class, and given the availability of small molecule ligands that recruit them, are the targets of most PROteolysis Targeting Chimeras (PROTACs)—heterobifunctional molecules for TPD generally comprising two small molecule ligands that bind separately to the E3 and the target protein, connected by a linker.

In some embodiments, screens were performed in parallel to find agents, e.g., Helicons, that bind to CRLs comprising Cullin proteins paired with their canonical adaptor proteins. In some embodiments, to identify and to assess agents, e.g., Helicons, that interact specifically with CRL1, CRL2, or CRL5, the N-terminal domains of CUL1, CUL2, and CUL5 were purified and screened in parallel with a counter-target CUL4B. In some embodiments, also included were the corresponding CRL substrate-recognition adaptors, including VHL and the ElonginB/ElonginC complex (ELOBC) for CUL2. In some embodiments, the present disclosure provides agents, e.g., Helicons, that form VHL-ELOBC-specific clusters and CUL5-specific clusters (see, e.g., FIG. 13, (D) and Table E11-2). In some embodiments, the present disclosure provides agents, e.g., Helicons, for CUL1, CUL2, FBXW7-SKP1, and SOCS2-ELOBC (see, e.g., Table E11-2).

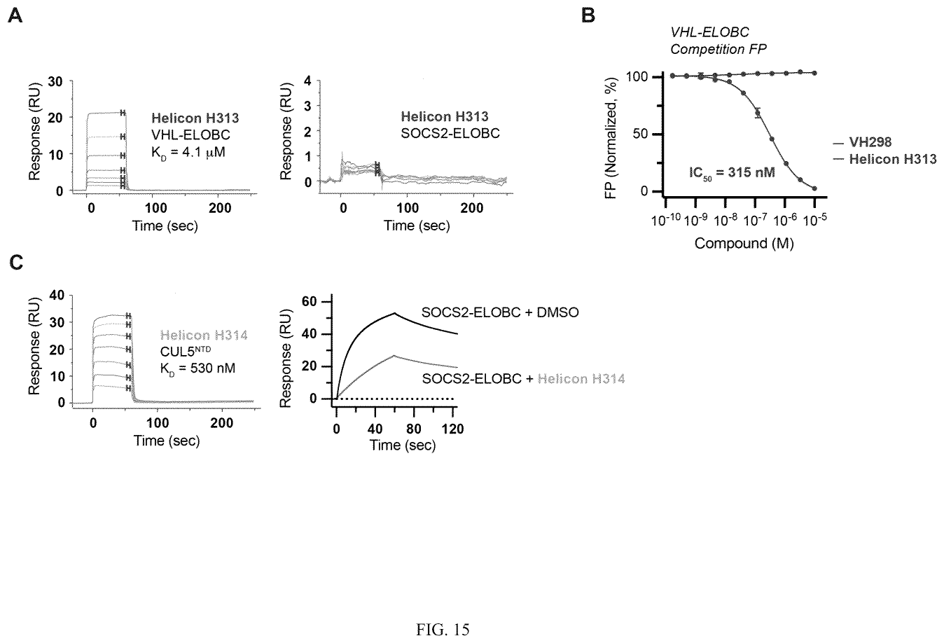

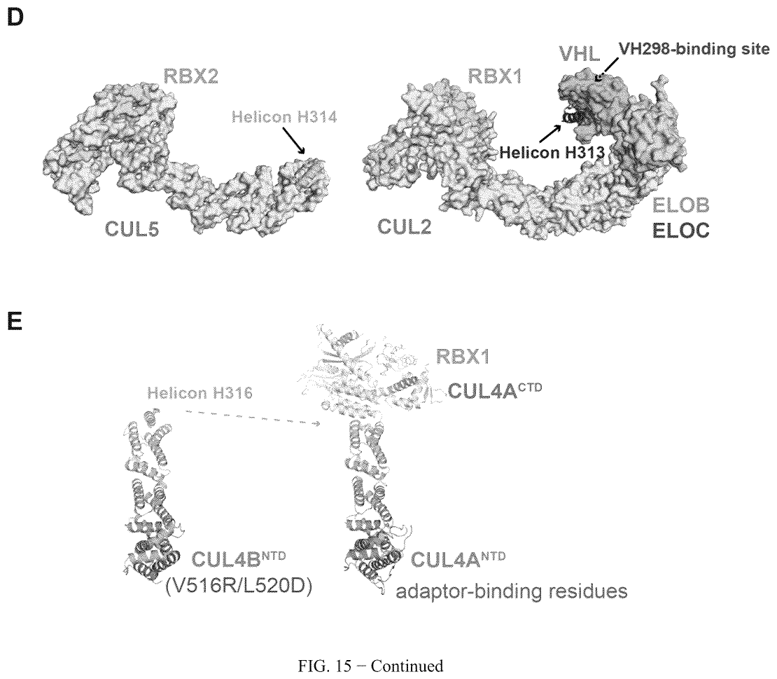

Various technologies can be utilized to characterize and assess agents of the present disclosure. In some embodiments, biochemical and biophysical approaches were utilized to confirm that the clusters and agents were target-specific and can bind to biologically relevant sites. Among the VHL-ELOBC-specific binding clusters, we identified Helicon H313 (C76) that binds VHL-ELOBC, but does not compete with the previously reported fluorescent VHL-binding probe, HXC78, and it did not bind SOCS2-ELOBC that acted as a counter-target for VHL-ELOBC in the CRL phage screens (Table E11-2). In some embodiments, the present disclosure provides technologies that bind VHL-ELOBC. In some embodiments, the present disclosure provides technologies that bind VHL-ELOBC selectively over SOCS2-ELOBC. In some embodiments, provided technologies do not compete with HXC78. In some embodiments, provided technologies compete with VH298. Using SPR, specificity of H313 for VHL-ELOBC over SOCS2-ELOBC was confirmed: the K_{D} for the former was calculated to be 4.1 μM (see, e.g., FIG. 15, (A)). A fluorescence polarization (FP) competition assay was utilized to show that H313 does not inhibit the HXC78 interaction with VHL-ELOBC, while the VHL ligand VH298, which is related to HXC78, does (see, e.g., FIG. 15, (B)). Among the CUL5-binding Helicons, H314 from C76 bound with high-affinity (K_{D}=530 nM), as assessed by SPR (see, e.g., FIG. 15, (C)). H314 also disrupts the SOCS2-ELOBC:CUL5 interaction in a competition SPR (ABA) binding assay. In some embodiments, provided technologies compete for the adaptor-binding site of CUL5 (see, e.g., FIG. 15, (C)). In some embodiments, the present disclosure provides technologies for disrupting SOCS2-ELOBC:CUL5 interaction.

In some embodiments, X-ray crystallography was utilized to characterize or assess provided technologies. A structure of H314 with the CUL5 N-terminal domain (CUL5^{NTD}) was solved at ˜2.8 Å resolution (see, e.g., FIG. 13, (E)), and H313 in complex with VHL-ELOBC at -3.5 Å resolution (see, e.g., FIG. 13, (F); and FIG. 15, (D)), and CUL4B-specific H316 in complex with the N-terminal domain of CUL4B (CUL4B^{NTD}) at ˜2.9 Å resolution (see, e.g., FIG. 15, (E)). In some embodiments, binding to a site revealed in the co-structure with CUL4B^{NTD} may not be relevant for certain biological functions. In some embodiments, a site is not available in the context of the CUL4B C-terminal domain (CUL4B^{CTD}) In some embodiments, biochemical and structural characterization are useful for assessing or confirming agents are of functional relevance, e.g., in cells.

Consistent with its disruption of the CUL5 interaction with ELOBC in SPR assays, H314 binds at the very N-terminus of CUL5, the binding site of the adaptor complex SOCS2-ELOBC (see, e.g., FIG. 15, (D)). In some embodiments, H313 binds at a unique VHL site that is distal to HXC78-binding site, the only known VHL ligand-binding site (see, e.g., FIG. 15, (D)).

In some embodiments, the distinct binding modes and Helicons described here can be further developed to provide additional probes and tools for this important E3 family. For instance, both the H314-binding site on CUL5 and the H313-binding site on VHL face the Ub-E2 binding sites of the cognate Cullin CTDs (see, e.g., FIG. 15, (D)), providing potentially ideal configuration for TPD applications, where ‘linkerology’ of heterobifunctional molecules is critically important to optimize ternary complex formation and the geometry of the E2- and E3-catalyzed reactions. In some embodiments, the present disclosure define functionally relevant binding sites on WWP E3 ligases which provides an opportunity to develop certain therapeutics for this target class.

Stapled Peptide Agents that Bind the RING/U-Box E3 Family

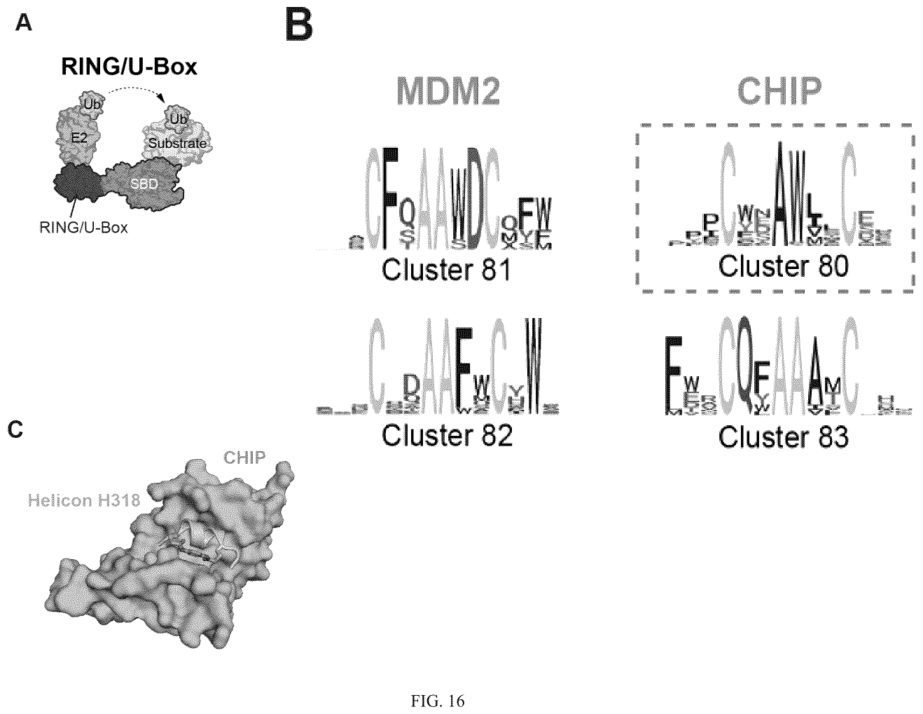

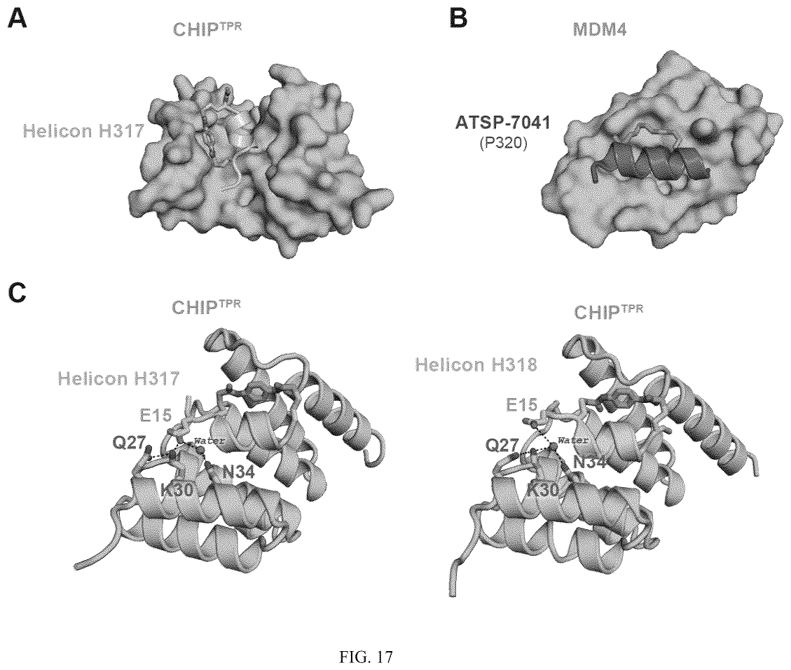

In some embodiments, the present disclosure provides technologies targeting RING/U-Box family (see, e.g., FIG. 16, (A)). In some embodiments, the present disclosure provides technologies that utilize MDM2 for targeted protein degradation. In some embodiments, the present disclosure provides technologies that utilize IAPs (inhibitor of apoptosis proteins) for targeted protein degradation. In some embodiments, the present disclosure provides technologies that utilize CHIP/STUB1 for targeted protein degradation. According to some reports, CHIP/STUB1 is more ubiquitously expressed (see, e.g., FIG. 14, (A)). In some embodiments, provided technologies induce degradation of relevant cancer targets. Certain Helicons that bind MDM2 and CHIP are presented in, e.g., Table E11-2 and FIG. 16 (B). In some embodiments, CHIP-binding Helicons H317 and H318 from C80, were further characterized and assessed, including by X-ray crystallography to examine binding to their E3 presenters (see, e.g., FIG. 16, (C); and FIG. 17, (A)). In some embodiments, MDM2-binding Helicon H319 from C81 was characterized. In some embodiments, H319 bound MDM2 with 3.7 μM K_{D}, as determined by SPR. In some embodiments, co-structure of H317 with CHIP, as well as the previously reported co-structure of MDM2 with ATSP-7041, suggest that various agents of the present disclosure in some instances may bind the E3s in a helical conformation (see, e.g., FIG. 17, (A) and (B)).

In some embodiments, agents of the same cluster may interact with targets similarly. In some embodiments, agents of the same cluster may interact with targets similarly but with differences. For example, in some embodiments, while Helicons H317 and H318 belonged to the same cluster, several differences in the co-structures with CHIP were observed, reflecting unique conformational changes and binding surfaces revealed by the Helicons. For instance, the side chain of Glu9 in H317 directly interacts with Lys30 of CHIP^{T}PR while Glu10 from H318 does not (see, e.g., FIG. 17 (C)). In some embodiments, given their size compared to small molecules, provided technologies such as stapled peptide agents, e.g., Helicons, can provide unique binding interfaces across their non-E3 facing surfaces, which can be used to bridge the E3 to target proteins, in some cases, in unique ternary complexes with new functionality.

### Trimerizer Technologies

In some embodiments, the present disclosure provides technologies for identifying, developing, optimizing, characterizing and/or assessing trimerizer agents, e.g., trimerizer Helicons. In some embodiments, based on their ability to present a much larger surface than small molecules that have been traditionally used to facilitate binding of E3s with substrate targets, provided agents comprising stapled peptides, e.g., Helicons, are engineered to cooperatively promote specific interactions between E3s and a range of substrate targets. In some embodiments, the present disclosure provides technologies to exploit large library diversity of a phage screening platform to explore a vast number of design hypotheses and/or to directly discover trimerizer Helicons, in some embodiments, without a need for structure-based design (see, e.g., FIG. 18, (A)).

In some embodiments, a provided approach for discovering trimerizer Helicons involves two successive screens of ˜10⁸-membered Helicon libraries, wherein the first is a naive binding screen (as described above for E3 ligases) and the second is a screen of a “focused” library that is designed based on hits from the naive screen, in some embodiments, comprises one or more enriched amino acid residues from a first screen. As confirmed herein, in some embodiments, hit clusters for the RING/U-Box family E3s CHIP and MDM2 were selected, and focused libraries were designed based on fixing these conserved binding residues and diversifying the exposed Helicon residues (see, e.g., FIG. 17, (B)). Among other things, it was observed and confirmed that in various cases, highly conserved/enriched positions in the sequence logos serve as accurate indicators of which residues are directly involved in target binding. In some embodiments, provided technologies leveraged this knowledge to design libraries where conserved/enriched positions in the logos for either CHIP or MDM2 were fixed, while the non-conserved residues were diversified. Trimerizer library primers were constructed designed using a combination of defined, semi-degenerate, and degenerate codons to match the sequence logo, in some embodiments, as closely as possible. In some embodiments, length of the Helicon library were extended to 20 amino acids to provide additional surface area for target binding.

Once constructed, these libraries were screened against target proteins in both the presence and absence of the E3 presenter they were designed to bind. In some embodiments, inspection of the phage binding data revealed two types of target binding behavior: binding that occurred regardless of the presence of the E3 presenter protein, and binding that occurred only when the E3 was present. In some embodiments, these latter, E3-dependent binders were selected for synthesis and validation. In some embodiments, E3 dependence indicated a cooperative binding mode to their target (see, e.g., FIG. 18, (C)). Those skilled in the art appreciate that the former agents may be useful for various purposing including binding target proteins and/or modulating functions thereof.

### Construction of Trimerizer Libraries for CHIP and MDM2

In some embodiments, focused phage libraries were designed and constructed based on selected CHIP and MDM2 hit clusters, including those exemplified by H319 for MDM2 and H317 and H318 for CHIP. In some embodiments, four total stapling and scaffolding residues across the 14-mer Helicons were fixed, and consensus residues responsible for binding defined by the cluster logos were also fixed (see, e.g., FIG. 18, (B)). In some embodiments, the remaining residues were randomized either fully (three to four residues at the ends of the Helicons, plus two internal residues that showed no amino acid preference in the cluster logos), or partially (residues that showed some preference for two to five amino acids), using degenerate or semi-degenerate codons, respectively, in the amplification primers (see, e.g., FIG. 18, (B)). In some embodiments, the focused library primers were synthesized and stapled phage libraries were generated in the same manner as the naive library used for the first screens.

For trimerizer phage screening, purified, bead-immobilized target proteins were incubated with the phage libraries—for example, the focused libraries built for discovery of CHIP and MDM2 trimerizers—but also included non-immobilized E3 presenter proteins free in solution during the screen (see, e.g., FIG. 18, (C)). In some embodiments, Helicon hits that promoted the formation of ternary complexes were identified. In some embodiments, the present disclosure provides agents that bind the target only in the presence of a presenter, e.g., an E3 ligase. In some embodiments, certain agents bind in a presenter-independent manner, e.g., an E3-independent manner. In some embodiments, the present disclosure provides presenter-dependent agents for targets. In some embodiments, a presenter is or comprises a polypeptide. In some embodiments, a polypeptide is or comprises an E3 ligase as described herein. In some embodiments, a target is or comprises a polypeptide. Various presenter-dependent agents were presented herein as examples. In some embodiments, the present disclosure provides presenter-dependent trimerizers for CHIP and TEAD4. In some embodiments, the present disclosure provides presenter-dependent trimerizers for CHIP and PPIA. In some embodiments, the present disclosure provides presenter-dependent trimerizers for MDM2 and β-catenin.

### Provided Technologies can Induce the Interaction Between CHIP and PPIA or TEAD4

In some embodiments, the present disclosure provides agents that can promote interactions between CHIP and a target polypeptide. In some embodiments, the present disclosure provides agents that can induce interactions between CHIP and a target polypeptide. In some embodiments, a polypeptide is PPIA. In some embodiments, a polypeptide is TEAD4.

According to some reports, TEAD4 is a member of the transcriptional enhancer factor (TEF) family of transcription factors, and through its interactions with YAP/TAZ, acts as an effector of the Hippo signaling pathway, which is implicated in cell proliferation and migration, organ development, and resistance to specific cancer treatments.

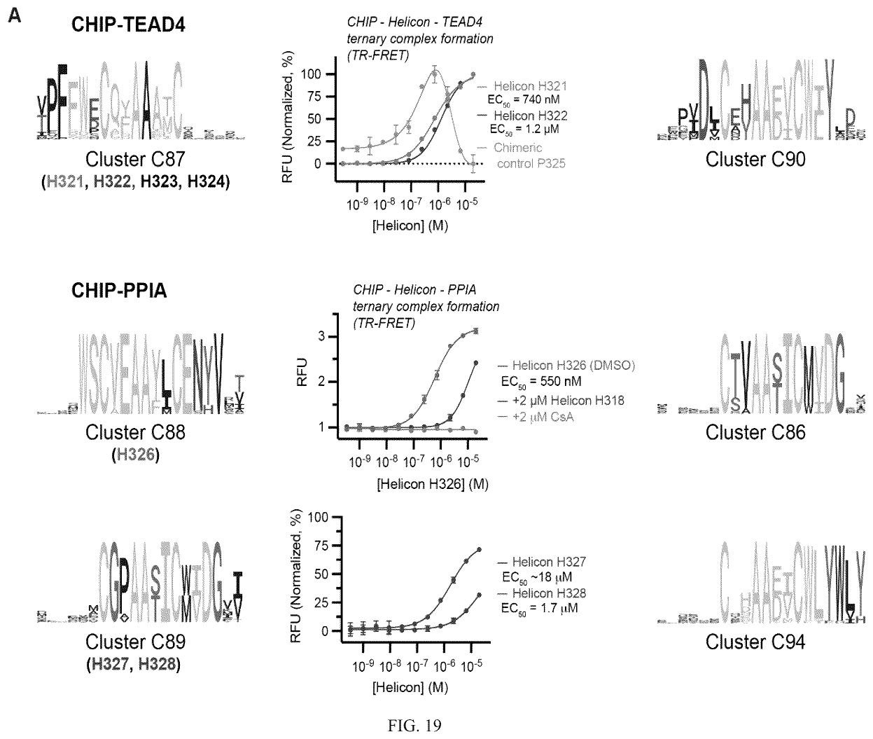

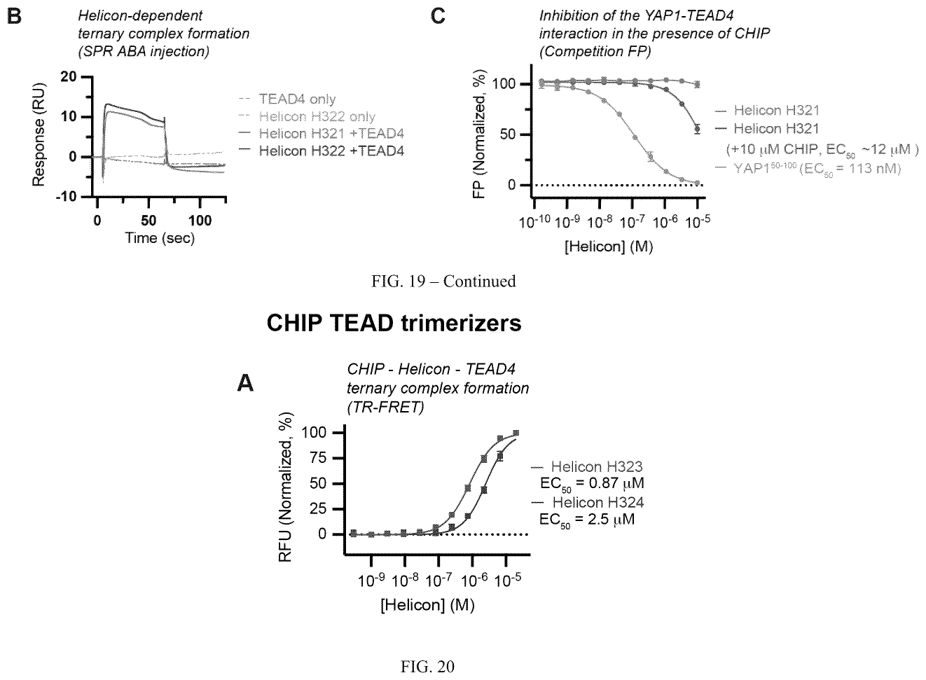

Various technologies can be utilized to assess complex formation induced by agents of present disclosure. For example, in some embodiments, capability of CHIP-TEAD4 trimerizers to promote ternary complex formation were assessed using time-resolved fluorescence energy transfer (TR-FRET), SPR (ABA mode), and fluorescence polarization (FP) assays (see, e.g., FIG. 19).

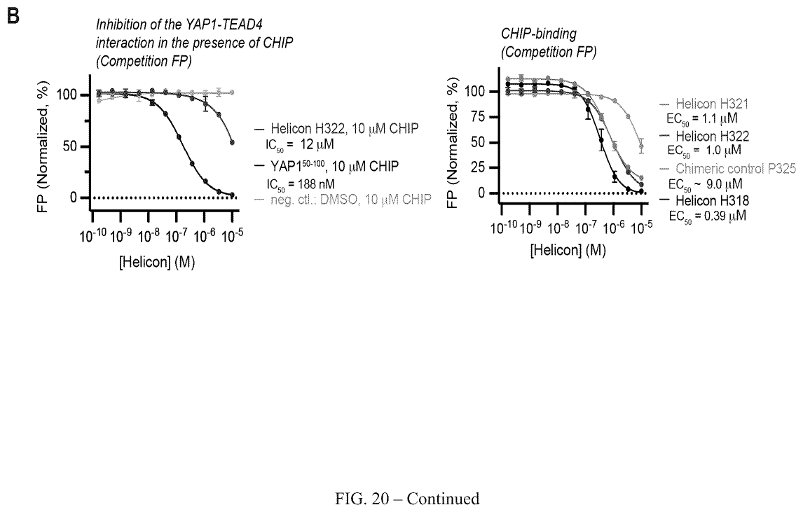

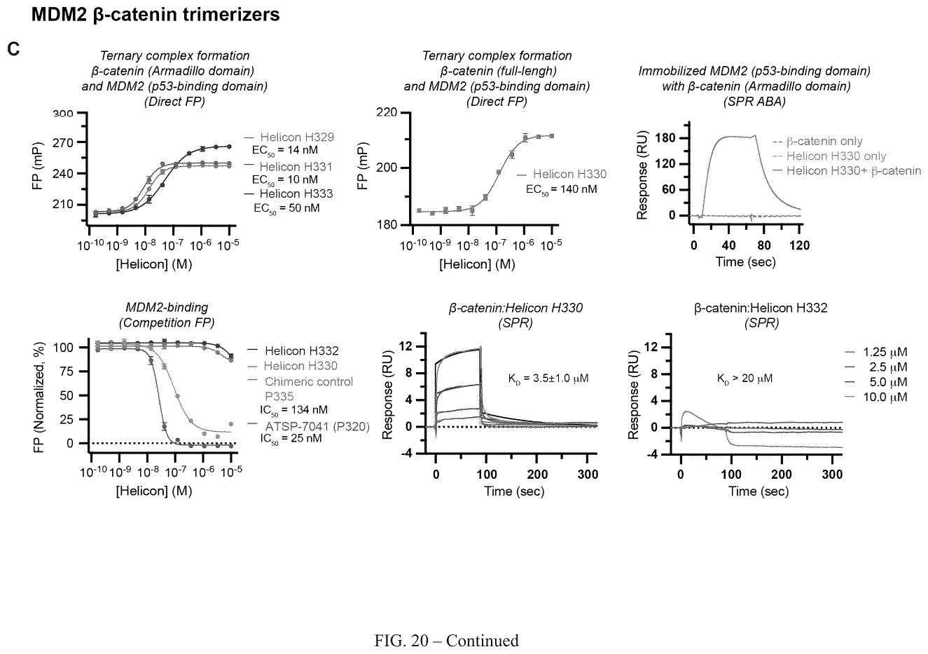

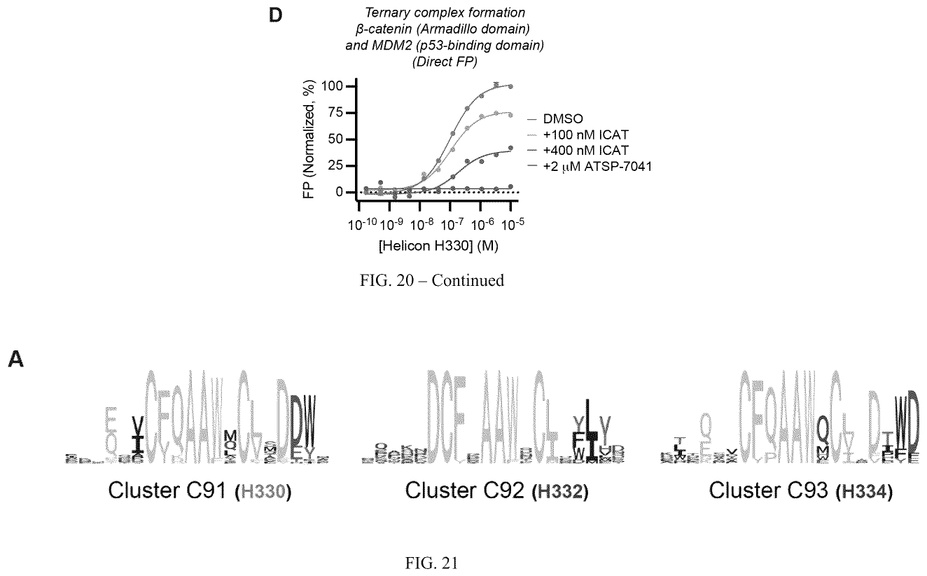

For TR-FRET, Helicon H321 from C85 and a C-terminally truncated version of H321, H322 were assessed (see, e.g., FIG. 19). We also tested Helicons H323 and H324 from the same cluster and a control peptide, P325, a heterobifunctional chimeric peptide generated by fusing CHIP- and TEAD4-interacting peptides (see, e.g., FIG. 20 (A)). Models of PROTAC-mediated ternary complex formation predict that high concentrations typically required for in vitro assays can saturate binding to the target, producing ineffective binary complexes that limit ternary complex formation, and result in a bell-shaped dependency on PROTAC concentration—a phenomenon referred to as the “hook effect”⁴³. Without the intention to be limited by theory, theoretically, the hook effect can be circumvented by improving the cooperative binding of the PPIs. In some embodiments, a hook effect with the chimeric P325 was observed, while H321-H324 showed clear dose-dependent trimerization over the range of concentrations we tested, consistent with cooperative formation of ternary complexes. Crucially, an ABA-format SPR assay confirmed that CHIP bound to TEAD4 only in the presence of H321 and H322 (see, e.g., FIG. 19, (B)).

In some embodiments, both H321 and H322 could disrupt the interaction between recombinant TEAD4 and a fluorescently labeled YAP1 fragment in the presence of CHIP in a competition FP assay (see, e.g., FIG. 19, (C); and FIG. 20, (B)). As expected, an unlabeled YAP1 fragment could also disrupt the interaction (see, e.g., FIG. 19, (C); and FIG. 20, (B)). In some embodiments, it was confirmed that while H321 and H322 did not interact with TEAD4 (see, e.g., FIG. 19, (B)), they did show weak binding affinity to CHIP (see, e.g., FIG. 20, (B)). In some embodiments, besides inducing ternary complex formation only in the presence of CHIP, CHIP-TEAD4 trimerizers can functionally disrupt the biologically relevant TEAD4-YAP1 interaction.

Among other things, technologies of the present disclosure provides versatility. For example, in some embodiments, versatility of CHIP related technologies to engage other targets beyond TEAD4 was confirmed. In some embodiments, the present disclosure provides trimerizers between CHIP and another polypeptide, e.g., PPIA (Cyclophilin A). PPIA is reported to be a peptidyl-prolyl cis-trans isomerase (PPIase) that plays a widespread role in the folding of nascent proteins. In some embodiments, screens with a CHIP-based trimerizer library identified several CHIP-dependent PPIA clusters, including C86, C88, C89, and C94 (see, e.g., FIG. 19, (A)). In some embodiments, Helicon H326 from C88 can promote ternary complex formation between CHIP and PPIA as shown by TR-FRET, and this was abolished by unlabeled CsA, and diminished by the CHIP-binder H318 described in e.g., FIG. 16, (C) (see, e.g., FIG. 19, (A)). In some embodiments, a complex may form via the CsA-binding site of PPIA or the H318-binding site of CHIP. C89 Helicons H327 and H328 can similarly act as trimerizers between the two proteins as shown by TR-FRET (see, e.g., FIG. 19 (A)).

### Provided Technologies can Induce the Interaction Between MDM2 and β-Catenin

In some embodiments, the present disclosure provides technologies that can induce interactions between MDM2 and target polypeptides. In some embodiments, a target polypeptide is beta-catenin. Certain useful technologies that modulate interactions between MDM2 and target polypeptides are described herein as examples, confirming, among other things, generality of provided technologies.

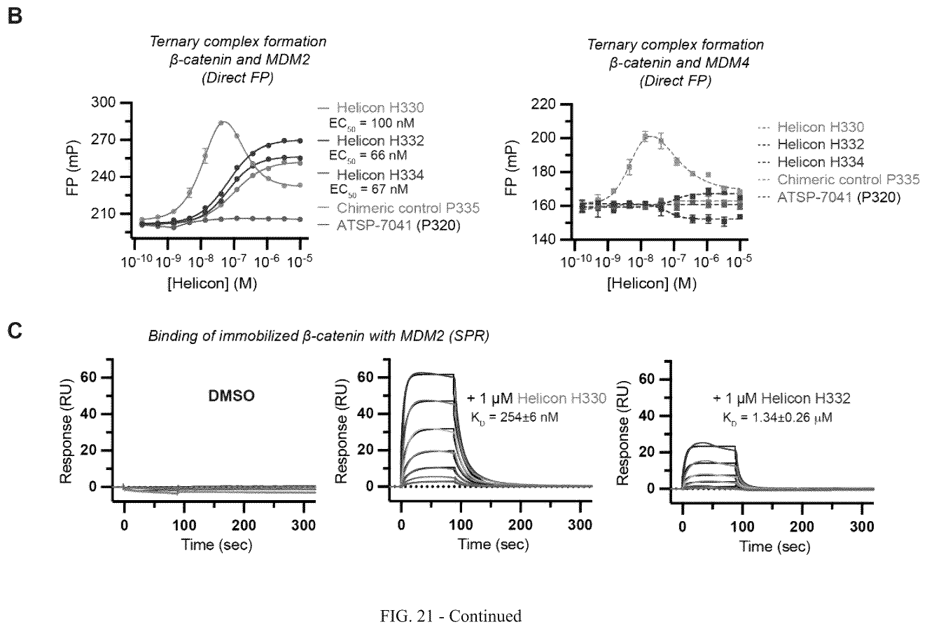

In some embodiments, MDM2-based libraries were screened against β-catenin, a key component of the canonical Wnt signaling pathway that is reported to be often dysregulated in cancer. In some embodiments, MDM2-focused 20-mer Helicon libraries were screened against β-catenin and hits were identified that bound β-catenin only in the presence of MDM2, which belonged to multiple clusters, including C91-C93 (see, e.g., FIG. 21, (A)). In some embodiments, representative Helicons from each of these three clusters were characterized for their ability to act as trimerizers by FP, SPR, and X-ray crystallography.

A direct FP assay of ternary complex formation confirmed that H329 and H330 from C91, H332 and H333 from C92, H334 from C93, and an N-terminally truncated version of H329 (H331) could all promote a cooperative interaction between MDM2 and the Armadillo domain of β-catenin with EC₅₀ values ranging from 10-100 nM, but not between MDM4 and β-catenin (see, e.g., FIG. 21, (B); and FIG. 20, (C)). Agents, e.g., H330, were also confirmed to promote the interaction between MDM2 and the full-length β-catenin (see, e.g., FIG. 20 (C)). A control heterobifunctional molecule P335 made by fusing MDM2- and β-catenin-interacting peptides exhibited a hook effect, and also promoted interactions between MDM4 and β-catenin (see, e.g., FIG. 21, (B)). B y competition FP, it was observed that both Helicon 330 and H332 possessed very weak binding to MDM2 alone (see, e.g., FIG. 20, (C)). By SPR, it was observed that H330, but not H332 could weakly interact with β-catenin in the absence of MDM2 (see, e.g., FIG. 20, (C)). Both H330 and H332 could promote the interaction between MDM2 and immobilized β-catenin with different affinities (see, e.g., FIG. 21, (C)).

An inverted SPR experiment further confirmed the H330 trimerizer activity, where MDM2 was immobilized with β-catenin as the free analyte (see, e.g., FIG. 20 (C)). The MDM2/MDM4-binding helical peptide ATSP-7041 did not promote ternary complex formation, consistent with the direct FP assay (see, e.g., FIG. 21, (B); and FIG. 20, (C)). H330 could compete with ATSP-7041 for binding to MDM2 and with β-catenin-binder ICAT for binding to β-catenin (FIG. 20 (D)). In some embodiments, provided technologies, e.g., H330, can bridge MDM2 and β-catenin via the ATSP-7041- and p53-binding site of MDM2 and the ICAT-binding site of β-catenin.

### Structural Characterization Confirms Direct Interactions Between all Three Molecules of the Complex

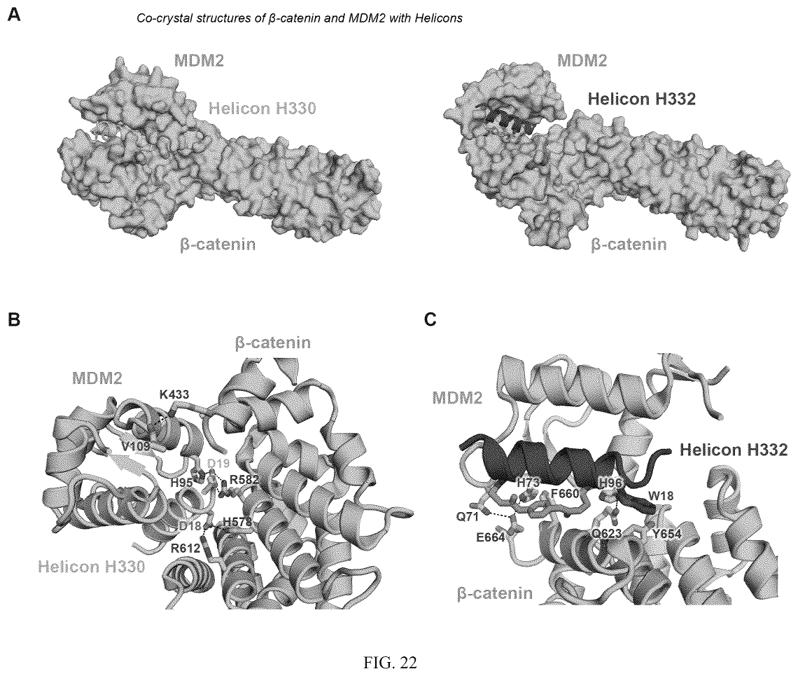

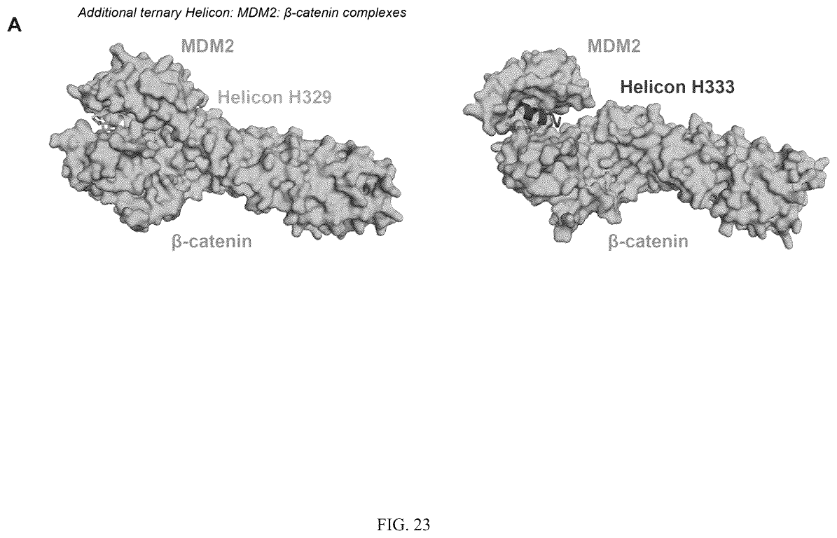

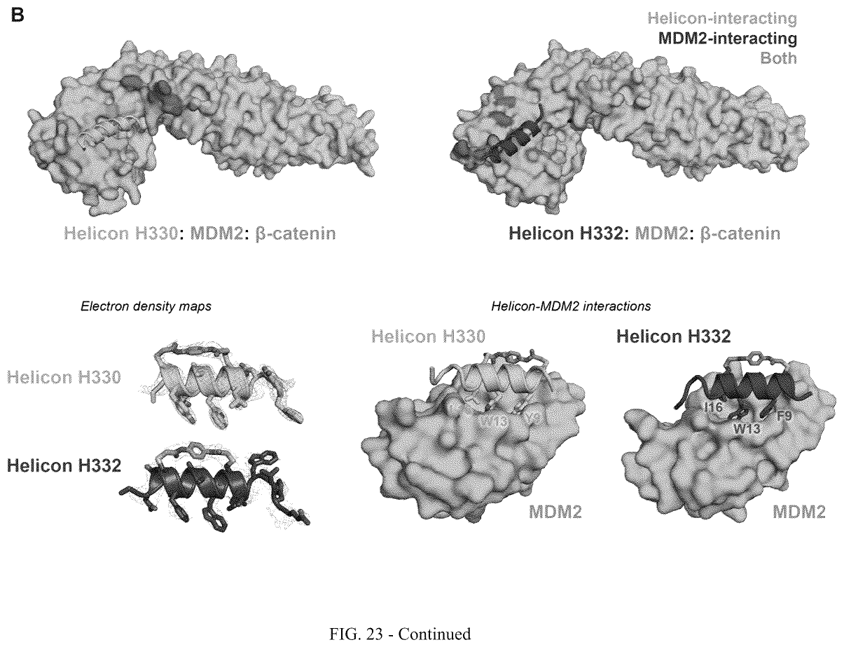

Various technologies can be utilized to characterize, assess and confirm formation of complexes formed by agents of the present disclosure and polypeptides, e.g., E3 ligase proteins and target proteins. In some embodiments, X-ray crystallography was utilized to characterize the MDM2-O-catenin complexes induced by trimerizer agents, e.g., H329 and H330 from C91 and H332 and H333 from C92. In some embodiments, calculated electron density maps indicated that residues 6-21 of H330 and residues 5-21 of H332 were well-resolved. (see, e.g., FIG. 22, (A); and FIG. 23, (A) and (B)). Of note, the structures of MDM2-O-catenin-H332 and MDM2-O-catenin-H333 were solved in different space groups but with identical crystal packings, excluding potential artifacts arising from crystallography. It was observed that binding surfaces from all four Helicons and the MDM2 surface contribute significantly to ternary complex formation, engaging the p53-binding site of MDM2 and the C-terminal ICAT-binding site of β-catenin. It was also observed that C91 and C92 Helicons engage different subsites and residues on the surface of the proteins.

Using the PDBePISA explorer to define the macromolecular interfaces between Helicons H330 and H332 and MDM2 or β-catenin, and between MDM2 and β-catenin, we observed that both Helicons induced similarly sized MDM2-O-catenin interfaces, though H330 did so with a much more extensive set of interactions with β-catenin than H332 did (see, e.g., Table E11-3; and FIG. 23 (B)).

In the 2.6 Å ternary structure between MDM2, Helicon H330, and β-catenin, we found that the sidechains of Tyr9, Trp13 and Leu16 of H330 insert deeply into a hydrophobic cleft on the MDM2 surface, reminiscent of the endogenous p53-MDM2 interaction mediated by p53 residues Phe19, Trp23, and Leu26 (see, e.g., FIG. 23, (B)). The interactions between H330 and β-catenin include a series of intramolecular hydrogen bonds, including between Arg582 of β-catenin and Asp19 of H330, between Arg612 of β-catenin and Asp18 of H330, and between His578 of β-catenin and Asp18 of H330 (FIG. 6B). Interestingly, Asp18 of H330 also directly interacts with His96 of MDM2, serving as a bridge for the assembly of the ternary complex. Also observed was direct crosstalk between the carbonyl group of MDM2 Val109 and the sidechain of β-catenin Lys433 (see, e.g., FIG. 22, (B)).

Similar to the H330-bridged ternary complex, the 3.9 Å ternary structure between MDM2, Helicon H332, and β-catenin also revealed the importance of Helicon residues Phe9, Trp13, and Ile16 for interacting with MDM2, but revealed a similar solution for MDM2-Helicon-binding to β-catenin, with notable differences at the β-catenin-binding interface (see, e.g., FIG. 22 (C) and FIG. 23, (B)). Specifically, the H332 0-catenin interaction is driven by the C-terminal hydrophobic tail residues of the Helicon, Trp18, and Ile19, engaging β-catenin residues including Trp654 (see, e.g., FIG. 22, (C)). Several Helicon-driven hydrogen bonds and salt bridges between MDM2 and β-catenin were observed, including Gln71 and His73 of MDM2 interacting with Glu664 of β-catenin, and His96 of MDM2 interacting with Gln623 of β-catenin. Phe665 of β-catenin was observed positioned in a hydrophobic pocket surrounded by His73 of MDM2 and the staple residue of H332 (see, e.g., FIG. 22, (C)). These results are consistent with H330 and H332 belonging to separate trimerizer clusters, with different exposed residues on the helix opposite the MDM2-binding surface.

In total, these structures reveal the structural basis for the trimerizer Helicon-induced molecular recognition events that promote cooperativity in binding between the E3 and target proteins. In some embodiments, provided technologies can allosterically modify surfaces of E3 ligases to shift their substrate selectivity.

Among other things, as confirmed herein the present disclosure provides technologies for targeting the E3 ligase family, which was considered largely undruggable by traditional small molecules, and even with the traction provided by TPD approaches, still today only a select handful of this large family have been targeted. In some embodiments, the present disclosure provides high-throughput screening platform to identify Helicons that bind, and in some cases modulate the function of diverse E3s across all four families, including the discovery of WWP and CUL binders as well as alternatives to existing VHL binders. These E3 ligases have diverse tissue distributions. In some embodiments, provided technologies can improve cellular selectivity and limit on-target but off-tissue toxicity of candidate therapeutics.

Among other things, various provided technologies, e.g., platform technologies, are rapid, affordable, scalable, and as confirmed with structurally diverse targets, they are generalizable, including here against a very large protein family. Beyond representing additional binding sites on E3s for their optimization into direct inhibitors and degrader-like tools, the trimerizer Helicons of the present disclosure confirmed that Helicons can bridge two proteins by increasing the surface area and cooperative interactions between them.

Provided technologies can overcome several shortcomings of other TPD tools such as PROTACs. As a drug modality, TPD tools such as PROTACs have several attractive features including superior selectivity and a catalytic-type mechanism of action that can result in total elimination of the target—including of disease-relevant point mutants and traditionally undruggable targets. As well, they are not subject to cellular resistance mechanisms, and they represent unique probes that can be used to gain new biological insights into the function of their targets. Unfortunately, PROTACs have several shortcomings. They are difficult to develop given the often-imbalanced determinants of ternary complex formation, proper distance and orientation to allow ubiquitin transfer between E2, E3 and the target protein, and cellular permeability. Typically, PROTAC design and development involves optimizing the linker between the E3- and target-engaging small molecules used to make them. Given the nature of linking these disparate molecules, it is difficult to program cooperative interactions, so PROTACs are prone to the hook effect, a phenomenon that limits ternary complex formation in in vitro assays used during the optimization process. Further, not all PROTACs that direct ternary complex formation lead to degradation. Target binding and ternary complex formation are not the only considerations for PROTAC development. The identity of the recruited E3 ligase also plays a critically important role in determining their degradation capacity.

Provided technologies have various features as tools for TPD and other applications requiring proximity-induced interactions. For example, they can promote cooperative interactions between polypeptides, e.g., E3s and target proteins to drive specificity. Provided technologies can also be developed across the entire E3 family, providing attractive alternatives to the E3s VHL, CRBN, MDM2, and IAP that are used in a majority of TPD applications. This includes the ability to precisely target a protein of interest in its native tissue with an E3 with a matched tissue distribution. Further, provided technologies significantly expand the range of E3-recruiting ligands that can be used. Generally, common E3-recruiting ligands, such as the molecular glues thalidomide and its derivatives are used across most TPD applications. Still further, provided technologies can present an orientation of the E3 ligase that allows for efficient ubiquitin transfer to target proteins.

In some embodiments, provided technologies can identify specific trimerizer hits across a broad range of E3-target interactions with a single round of focused screening. In some embodiments, leads are optimized for the cellular penetration of the leads. It is noted a number of peptides have been used as PPI inhibitors, including ALRN-6924, an analog of ATSP-7041, the chimeric control utilized herein. It is reported that ALRN-6924 inhibits the degradation of the tumor suppressor p53 by targeting the E3s MDM2 and MDM4. There are also reports that viral proteins such as HIV-1 Vif can recruit host E3s and rewire their substrate specificities to counteract immunity. The F-box domains from HIV-1 Vif, VHL and SOCS2 have been reported to share a high structural homology and all bind the RING E3 ligase CUL5-ELOBC complex.

In some embodiments, directed evolution are utilized to exploit the E3-binding capability of Helicons to increase the surface area for binding target proteins. In some embodiments, the present disclosure provides focused libraries from E3-binding hits. In some embodiments, such libraries are screened against therapeutically important target proteins for those that bind only in the presence of the E3 presenter protein. In some embodiments, the successful identification of trimerizers that bridge E3s with target proteins greatly expands the range of tools available for applications requiring induced PPIs, and may eliminate the potential dose-limiting viabilities of current molecules in this space.

PPIs are among the most intractable targets for probes and drugs, and being able to use Helicons to not only disrupt these but also to generate novel PPIs, highlights certain strength of provided technologies to advance these fields simultaneously.

Atomic coordinates and structure factors have been deposited in the Protein Data Bank with accession codes, e.g., 8EIC, 8EHZ, 8EI0, 8EI1, 8EI2, 8EI3, 8EI4, 8EI5, 8EI6, 8EI7, and 8EI8. Certain results are also presented below:

TABLE E11-3
Certain macromolecular interfaces driven by trimerizer Helicons.

| Buried | | | |
|---|---|---|---|
| Interface (Å²) | Helicon:β-catenin | Helicon:MDM2 | MDM2:β-catenin |
| H330-mediated | 640 | 700 | 340 |
| MDM2:β-catenin | | | |
| interaction | | | |
| H332-mediated | 280 | 780 | 400 |
| MDM2:β-catenin | | | |
| interaction | | | |

### Certain Useful Technologies

Those skilled in the art appreciate that many technologies may be utilized for preparing, characterizing, assessing and/or using agents of the present disclosure. Certain technologies are described herein as examples.

### Recombinant Protein Expression.

Unless otherwise stated, all protein constructs correspond to human protein sequences.
WWP^{WW-HECT}, WWP1^{HECT} and WWP2^{HECT}

The expression and purification of human WWP1 (Uniprot ID: Q9HOMO) and WWP2 (Uniprot ID: 000308) fragments were adapted from previous work (Wang, Z. et al. A multi-lock inhibitory mechanism for fine-tuning enzyme activities of the HECT family E3 ligases. Nat Commun 10, 3162 (2019)). Briefly, WWP1^{WW-HECT} (residues 379-922), WWP1^{HECT} (residues 546-917) and WWP2^{HECT} (residues 492-865) were individually cloned into pET-based expression vectors to generate the final constructs GST-TEV-WWP1³⁷⁹⁻⁹²²-yBBr, His-TEV-WWP1⁵⁴⁶⁻⁹¹⁷-yBBr, and GST-TEV-WWP2⁴⁹²⁻⁸⁶⁵-yBBr, respectively, for phage screening and SPR analysis; and His-Thrombin-WWP1⁵⁴⁶⁻⁹¹⁷, His-3C-WWP2⁴⁹²⁻⁸⁶⁵ for ELISA and crystallography. Recombinant proteins were expressed in Escherichia coli BL21 (DE3) (New England Biolabs). After induction at 16° C. for 16 hrs with 1 mM isopropyl R-D-1-thiogalactopyranoside (IPTG), the cells were harvested and resuspended in buffer containing 50 mM Tris pH 8.0, 500 mM NaCl, 10% glycerol, and 1 mM phenylmethylsulfonyl fluoride (PMSF). For purification, the pellet was lysed with a tip sonicator, and centrifuged at 22,000×g for 30 mins at 4° C. The supernatant was purified with Pierce™ Glutathione Agarose or Ni-NTA resin (Qiagen), eluting with 50 mM Tris pH 8.0, 500 mM NaCl, 1 mM tris(2-carboxyethyl) phosphine (TCEP), 10% glycerol, with 10 mM reduced glutathione (GSH) or 250 mM imidazole. Eluted proteins were pooled, concentrated, and cleaved by adding the corresponding protease at a protease to protein ratio of 1:10 and incubated overnight at 4° C. yBBr-tag-containing proteins were biotinylated via the yBBr reaction according to standard procedures⁶². Final proteins were loaded onto a Superdex™ 10/300 75 pg or 200 pg (Cytiva) size exclusion chromatography (SEC) column and eluted in 50 mM Tris, pH 8.0, 200 mM NaCl, 10% glycerol, 1 mM DTT, and 1 mM EDTA. Fractions containing target protein were collected and pooled. GST contaminants were removed with an additional GST-purification step with target protein collected in the flow-through. Final protein fractions were concentrated and stored at −80° C.

The yBBr reaction was carried out as previously described (Yin, J. et al. Genetically encoded short peptide tag for versatile protein labeling by Sfp phosphopantetheinyl transferase. Proc National Acad Sci 102, 15815-15820 (2005)) with 100 μM target protein tagged with ybbR13 (DSLEFIASKLA (SEQ ID NO: 41)), incubated with 150 μM CoA-PEG11-biotin, 5 μM Sfp, and 10 mM MgCl₂ in protein storage buffer at room temperature for 1 hr. Excess CoA-conjugates and Sfp enzymes were removed by follow-up SEC.

### Chemical Structure of CoA-PEG11-Biotin:

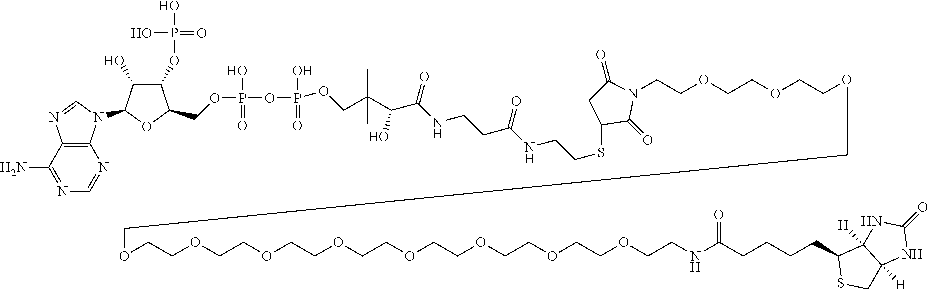 [SMILES: CC(C)(COP(=O)(O)OP(=O)(O)OC[C@H]1O[C@@H](n2cnc3c(N)ncnc32)[C@H](O)[C@@H]1OP(=O)(O)O)[C@@H](O)C(=O)NCCC(=O)NCCSC1CC(=O)N(CCOCCOCCOCCOCCOCCOCCOCCOCCOCCOCCOCCNC(=O)CCCC[C@@H]2SC[C@@H]3NC(=O)N[C@@H]32)C1=O]

### N-Terminal Domains of CUL1, CUL2, CUL4B, and CUL5

For proteins used in phage screening and SPR: the N-terminal domains of CUL1 (Uniprot ID: Q13616, residue 15-410, V367R/L371D), CUL2 (Uniprot ID Q13617, residues 8-384, V340R/L344D), CUL4B (Uniprot ID: Q13620, residues 206-557, V516R/L520D), and CUL5 (Uniprot ID: Q93034, residues 1-386, V341R/L345D) with N-terminal GST-TEV tags and C-terminal AVI tags were cloned into a pET-derived expression vector (Novagen). For proteins used in crystallography: the N-terminal domains of CUL5 (residues 8-384, V340R/L344D) or CUL4B (residues 206-557, V516R/L520D) with N-terminal GST-TEV tags were cloned into pET21b. Proteins were recombinantly expressed in E. coli BL21 CodonPlus cells (Agilent). The cells were induced at OD=0.6 with 1 mM IPTG for 4 hrs at 37° C., then harvested and resuspended in buffer, 20 mM HEPES pH 7.5, 300 mM NaCl, 10% glycerol, and 1 mM PMSF. For purification, the pellet was lysed with a tip sonicator, toggling between 3 sec on and 3 sec off for 20 min, and pelleted at 22,000×g for 30 min. at 4° C. The supernatant was purified using Pierce™ Glutathione Agarose resin eluting with 20 mM HEPES pH 7.5, 300 mM NaCl, 10% glycerol, and 10 mM GSH. Eluted proteins were pooled, concentrated, and cleaved by adding TEV protease at a ratio of 1:10 protease to protein and incubated overnight at 4° C. TEV-cleaved proteins were biotinylated with the published AviTag™ technology (Tykvart, J. et al. Efficient and versatile one-step affinity purification of in vivo biotinylated proteins: Expression, characterization and structure analysis of recombinant human glutamate carboxypeptidase II. Protein Expres Purif 82, 106-115 (2012)). Briefly, purified target proteins were incubated with BirA biotin ligase with a 20:1 molar ratio, in a reaction buffer containing 50 μM biotin, M ATP and 10 mM MgCl₂ at 4° C. for 16 hrs proteins were loaded onto a Superdex™ 10/300 75 pg (Cytiva) SEC column and eluted in 20 mM Tris pH 7.4, 200 mM NaCl, 2 mM DTT, and 5% glycerol. Fractions containing target protein were collected and pooled. GST contaminants were removed with an additional GST-purification step with target protein collected in the flow-through. Final protein fractions were concentrated and stored at −80° C.

### VHL, SOCS2, and FBWX7

For protein used in phage display screens and SPR: FBXW7 (Uniprot ID: Q969H0, residues 263-706) with an N-terminal GST-TEV tag and C-terminal AVI tag were co-expressed with full-length SKP1 (Uniprot ID: P63208, residue 1-163) in the pETDuet-1 plasmid. SOCS2 (Uniprot ID: 014508, residues 32-198) or VHL (Uniprot ID: P40337, residues 54-213) with an N-terminal 6×His-TEV tag (“HHHHHH” disclosed as SEQ ID NO: 39) cloned into pET21b and co-expressed with C-term AVI-tagged ELOB (Uniprot ID: Q15370, residues 1-104) and ELOC (Uniprot ID: Q15369, residues 17-112) cloned in pCDFDuet-1. For protein used in competition SPR (ABA mode) and x-ray crystallography: SOCS2 (residues 32-198) or VHL (residues 54-213) with an N-terminal 6×His-TEV tag (“HHHHHH” disclosed as SEQ ID NO: 39) cloned in pET21b was co-expressed with full-length ELOB (residues 1-118) and ELOC (residues 17-112) cloned in pCDFDuet-1. Recombinant proteins were expressed in E. coli BL21 (DE3) host cells and purified and biotinylated as for the WWP and Cullin proteins above.

### MDM2 and MDM4

For proteins used in the phage display screens and SPR: the p53-binding domain of MDM2 (Uniprot ID: Q00987, residues 25-109) with an N-terminal 6×His-yBBr-TEV tag (“HHHHHH” disclosed as SEQ ID NO: 39) was recombinantly expressed in E. coli BL21 CodonPlus cells (Agilent) from pET-derived expression vectors (Novagen). The cells were induced at OD₆₀₀=0.6 with 1 mM IPTG for 4 hrs at 37° C., then harvested and resuspended in 25 mM Tris-HCl pH 7.5, 300 mM NaCl, 10% glycerol, 1 mM PMSF. For purification, the pellet was lysed with a tip sonicator, and pelleted at 22,000×g for 30 mins at 4° C. The pellets were washed three times with 20 mM Tris-HCl pH 8.0, 150 mM NaCl, 1 M urea, 1.0% Triton X-100, and dissolved in 20 mM Tris-HCl pH 8.0, 150 mM NaCl, 8 M urea, and 2 mM B-mercaptoethanol (B-me). The supernatant was purified using a Ni-NTA resin (Qiagen), and eluted with 20 mM Tris-HCl pH 8.0, 150 mM NaCl, 8 M urea, 2 mM B-me, and 250 mM imidazole. Protein elutes were diluted to ˜0.1 mg/mL and dialyzed into buffers containing 10 mM Tris-HCl pH 8.0, 150 mM NaCl, 2 mM B-me, with 4, 2, 1, or 0 M urea, at 4° C. for 8 hr for each urea gradient. Urea-free proteins were concentrated with Amicon spin filters (Millipore Sigma) to ˜1 mg/mL and biotinylated via the yBBr reaction according to standard procedures (Yin, J. et al. Genetically encoded short peptide tag for versatile protein labeling by Sfp phosphopantetheinyl transferase. Proc National Acad Sci 102, 15815-15820 (2005)), as above. Biotinylated proteins were pooled, concentrated, and loaded onto a Superdex™ 10/300 75 pg (Cytiva) SEC column and eluted in 20 mM HEPES pH 7.0, 200 mM NaCl, 5% glycerol, 0.5 mM TCEP. Fractions containing pure protein were collected, pooled, concentrated to ˜1 mg/mL and stored at −80° C.

For protein used in crystallography and other biochemical assays: p53-binding domains of MDM2 (residues 17-111, with C17S substitution; MDM2¹⁷⁻¹¹¹) with an N-terminal 6×His-TEV tag (“HHHHHH” disclosed as SEQ ID NO: 39), and MDM4 (Uniprot ID: O15151, residues 14-111, with C17S substitution; MDM4¹⁴⁻¹¹¹) with an N-terminal 6×His-yBBr-3C tag (“HHHHHH” disclosed as SEQ ID NO: 39) were recombinantly expressed in E. coli BL21 (DE3) cells (Agilent) from pET-derived expression vectors (Novagen). The cells were induced at OD₆₀₀=0.6 with 0.15 mM IPTG for 16 hr at 37° C., then harvested and resuspended in 50 mM Tris, pH 8.0, 200 mM NaCl, 10% glycerol, 1 mM TCEP, and 20 mM imidazole. For purification, the pellet was lysed with a tip sonicator, toggling between 3 sec on and 3 sec off for 20 min, and then centrifuged at 22,000×g for 30 min at 4° C. The supernatant was purified using a Ni-NTA resin (Qiagen), and eluted with 50 mM Tris, pH 8.0, 200 mM NaCl, 10% glycerol, 1 mM TCEP, and cleaved by adding protease (TEV or PreScission protease) at a protease to protein ratio of 1:10 and incubated overnight at 4° C. Cleaved proteins were loaded onto a Superdex™ 10/300 75 pg (Cytiva) SEC column and eluted in 50 mM Tris, pH 8.0, 200 mM NaCl, 10% glycerol, and 1 mM TCEP. Fractions containing pure protein were collected, pooled, concentrated to ˜8 mg/mL and stored at −80° C.

For protein labeling: Purified tag-free MDM2¹⁷⁻¹¹¹ and MDM4¹⁴⁻¹¹¹ were loaded onto a Superdex™ 10/300 75 pg (Cytiva) SEC column and eluted in 25 mM HEPES pH 7.5 and 250 mM NaCl, with pooled protein factions with the concentration at 300 μM. The protein was then mixed with Alexa Fluor™ 488 NHS Ester (Thermo Scientific) prepared as 100 mM stock, with a final protein to NHS ratio of 1:0.8. The reaction was carried out at room temperature and quenched with 50 mM hydroxylamine before the final SEC purification in 25 mM HEPES pH 7.5, 250 mM NaCl and 1 mM TCEP buffer. Fractions containing Alexa488-labeled protein were collected, pooled, and stored at −80° C.

### CHIP

For protein used in phage screen and SPR: N-term truncated CHIP (also known as STUB1, Uniprot ID: Q9UNE7, residues 23-303; CHIP²³-3⁰³) or the TPR domain of CHIP (residues 23-154; CHIP²³-15) with an N-terminal 6×His-yBBr-TEV tag (“HHHHHH” disclosed as SEQ ID NO: 39) was recombinantly expressed in E. coli BL21 CodonPlus cells (Agilent) from pET-derived expression vectors (Novagen). The cells were induced at OD₆₀₀=0.6 with 1 mM IPTG for 4 hrs at 37° C., then harvested and resuspended in 50 mM Tris-HCl pH 8.0, 500 mM NaCl, 10 mM imidazole, 10% glycerol, and 10 mM 0-me. For purification, the pellet was lysed with a tip sonicator and centrifuged at 22,000×g for 30 mins at 4° C. The supernatant was purified with Ni-NTA resin (Qiagen), eluted with 50 mM Tris-HCl pH 8.0, 500 mM NaCl, 10% glycerol, 10 mM R-me and 250 mM imidazole, and biotinylated via the yBBr reaction according to standard procedures as above. Biotinylated proteins were pooled, concentrated, and loaded onto a Superdex™ 10/300 75 pg (Cytiva) SEC column, and eluted in 20 mM HEPES pH 7.0, 150 mM NaCl, 10% glycerol, 2 mM DTT. Fractions containing pure protein were collected, pooled, concentrated to ˜1.2 mg/mL and stored at −80° C.

For proteins used in crystallography and other biochemical assays: CHIP TPR domain, CHIP²¹⁻¹⁵⁴ or CHIP²³-1⁵⁴, or CHIP²³-3⁰³ each with an N-terminal 6×His-TEV tag (“HHHHHH” disclosed as SEQ ID NO: 39) were recombinantly expressed in E. coli BL21 CodonPlus cells (Agilent) from a pET21b-derived expression vector (Novagen). The cells were induced at OD₆₀₀=0.6 with 1 mM IPTG for 4 hrs at 37° C. or 16 hrs at 16° C., then harvested and resuspended in 50 mM Tris-HCl pH 8.0, 500 mM NaCl, 10 mM imidazole, 10% glycerol, and 10 mM B-me. For purification, the pellet was lysed with a tip sonicator, toggling between 3 sec on and 3 sec off for 20 min, centrifuged at 22,000×g for 30 mins at 4° C. The supernatant was purified with Ni-NTA resin (Qiagen), eluting with 250 mM imidazole. Eluted proteins were pooled, concentrated, and the TEV tag cleaved off by adding TEV protease at a protease to protein ratio of 1:10 and incubated overnight at 4° C. Cleaved/untagged proteins were loaded onto a Superdex™ 10/300 75 pg (Cytiva) SEC column, and eluted in 50 mM HEPES, pH 8.0, 150 mM NaCl, 10% glycerol, 2 mM DTT. Fractions containing pure protein were collected, pooled, concentrated to −30 mg/mL and stored at −80° C.

For Alexa488 labeling: Purified tag-free CHIP²³-3⁰³ was labeled as MDM2¹⁷¹¹¹ above, with the final SEC purification in buffer: 20 mM Tris pH 7.5, 250 NaCl, and 1 mM DTT. Fractions containing Alexa488-labeled protein were collected, pooled, concentrated to 0.6 mg/mL, and stored at −80° C.

### PPIA

Full length PPIA (Uniprot ID: P62937, residues 1-165) with an N-terminal 6×His-yBBR-TEV tag (“HHHHHH” disclosed as SEQ ID NO: 39) was recombinantly expressed in E. coli BL21 (DE3) CodonPlus RIPL cells (Agilent) from pET-derived expression vectors (Novagen). The cells were induced at OD₆₀₀=0.6 with 0.15 mM IPTG for 16 hr at 16° C., then harvested and resuspended in PBS pH 7.4 with 1 mM PMSF. For purification, cell pellets were lysed with a tip sonicator and centrifuged at 22,000×g for 30 min at 4° C. The resulting supernatant was collected then centrifuged again at 22,000×g for 30 min at 4° C. The supernatant was purified with Ni-NTA resin (Qiagen) and eluted with 250 mM imidazole. TEV was cleaved from the recombinant proteins by adding TEV protease at a protease to protein ratio of 1:10 and incubation for 4 hr at 4° C. Protein was then concentrated and diluted into 20 mM HEPES pH 7.0, 5% glycerol, and centrifuged at 22,000×g for 10 min at 4° C. The supernatant was loaded onto an SP HP (Cytiva) column pre-equilibrated with 20 mM HEPES pH 7.0, 5% glycerol. Purified protein was eluted with a gradient from 0 mM to 1 M NaCl. Protein fractions were pooled, concentrated then centrifuged at 22,000×g for 10 min at 4° C. The supernatant was collected and loaded onto a Superdex™ 16/600 75 pg (Cytiva) SEC column pre-equilibrated with PBS pH 7.4. Purified proteins were eluted isocratically in PBS pH 7.4. Protein fractions were collected, concentrated, aliquoted and frozen.

### TEAD4

YAP/TAZ binding domain of TEAD4 (Uniprot ID: Q15561, residues 217-434) with an N-terminal 6×His-yBBr-TEV tag (“HHHHHH” disclosed as SEQ ID NO: 39) was recombinantly expressed in E. coli BL21-CodonPlus cells (DE3) (Agilent) from pET-derived expression vectors (Novagen). The cells were induced at OD₆₀₀=0.6 with 0.15 mM IPTG for 16 hrs at 16° C., then harvested and resuspended in 50 mM Tris pH 7.4, 200 mM NaCl, 5% glycerol, 1 mM TCEP, 1 mM PMSF. For purification, the pellet was lysed with a tip sonicator, pelleted at 22,000×g for 30 min at 4° C., then the supernatant was purified with an Ni-NTA column (Cytiva), eluting with 250 mM imidazole. Protein-containing fractions were pooled, concentrated, and loaded onto a Superdex® 75 10/300 (Cytiva) SEC column. Purified proteins were eluted isocratically in 50 mM Tris pH 7.4, 200 mM NaCl, 5% glycerol, 1 mM TCEP, and fractions containing pure protein were collected, pooled and frozen.

β-catenin (CTNNB1)

β-catenin protein (encoded by CTNNB1, Uniprot ID: P35222) Armadillo domain (residues 134-665) with a N-terminal 6×His-yBBr-TEV tag (“HHHHHH” disclosed as SEQ ID NO: 39) was recombinantly expressed in E. coli BL21 (DE3) pLysS cells (Thermo Fisher) from pET28a vectors (Novagen). The cells were induced at OD₆₀₀=0.6 with 0.15 mM IPTG for 16 hrs at 16° C., then harvested and resuspended in 25 mM Tris pH 8.0, 200 mM NaCl, 10% glycerol, 20 mM imidazole, 1 mM PMSF. For purification, the pellet was lysed with a tip sonicator, centrifuged at 22,000×g for 30 min at 4° C., then the supernatant was purified with HisTrap HP columns (Cytiva), eluting with 250 mM imidazole. For crystallography and selected biochemical assays, protein was TEV-cleaved by adding TEV protease at a protease to protein ratio of 1:10 and incubated overnight at 4° C. For phage display screening and SPR analysis, protein was biotinylated via the yBBr reaction according to standard procedures. All proteins were concentrated using Amicon spin filters (Millipore Sigma) then diluted into 25 mM Tris, pH 8.8, 1 mM DTT, 10% glycerol and loaded onto a Q HP (Cytiva) column. Proteins were eluted with a gradient from 50 mM to 600 mM NaCl. Protein-containing fractions were pooled, concentrated and loaded onto a Superdex™ 10/300 200 pg (Cytiva) SEC column. Purified proteins were eluted isocratically in 25 mM Tris-HCl, pH 8.8, 10% glycerol, 300 mM NaCl, and fractions containing pure protein were collected and pooled.

Full-length β-catenin protein (CTNNB1, residues 1-781) with N-terminal 6×His-thrombin-T7-TEV tag (“HHHHHH” disclosed as SEQ ID NO: 39) was recombinantly expressed in E. coli BL21 (DE3) pLysS cells (Thermo Fisher) from pET28a vectors (Novagen). E. coli cells were induced at OD₆₀₀=0.6 with 0.15 mM IPTG for 20 hrs at 16° C., shaking at 180 rpm, then harvested and resuspended in 20 mM HEPES, pH 7.5, 300 mM NaCl, 10% glycerol. For purification, the pellet was lysed with a sonicator, pelleted at 130,000 rpm for 30 mins at 4° C., then the supernatant was purified with a Ni-NTA column (Cytiva), eluting with 250 mM imidazole. Protein was then diluted into 20 mM HEPES, pH 7.5, 10% glycerol, 1 mM DTT and loaded onto a Q HP column (Cytiva), and was eluted with a NaCl gradient of concentration 0 to 1.0 M. Protein-containing fractions were pooled, concentrated, and loaded onto a Superdex™ 10/300 200 pg (Cytiva) SEC column. Purified protein was eluted isocratically in 20 mM HEPES, pH7.5, 300 mM NaCl, 10% glycerol and 1 mM DTT, and fractions containing pure protein were collected and pooled.

### ICAT (CTNNBIP1)

Full-length ICAT (CTNNBIP1, Uniprot ID: Q9NSA3, residues 1-81) with an N-terminal GST-TEV tag was recombinantly expressed in E. coli BL21-CodonPlus cells (DE3) (Agilent) from pET-derived expression vectors (Novagen). The cells were induced at OD₆₀₀=0.6 with 0.15 mM IPTG for 16 hrs at 16° C., then harvested and resuspended in 20 mM Tris pH 7.4, 200 mM NaCl, 10% glycerol, 0.5 mM TCEP, 1 mM PMSF. For purification, the pellet was lysed with a tip sonicator, pelleted at 22,000×g for 30 min at 4° C., then the supernatant was purified with a GST column (Cytiva), eluting with 10 mM glutathione. Protein-containing fractions were pooled, concentrated, and loaded onto a Superdex™ 75 10/300 (Cytiva) SEC column. Purified proteins were eluted isocratically in 20 mM Tris pH 7.4, 200 mM NaCl, 10% glycerol, 0.5 mM TCEP and fractions containing pure protein were collected, pooled and frozen.

Trimerizer Phage Library construction (e.g., primers, protocol, crosslinking, and DNA sequencing, etc.)

Certain useful technologies are described in Li, K. et al. De novo mapping of α-helix recognition sites on protein surfaces using unbiased libraries. Proc National Acad Sci 119, e2210435119 (2022), and can be utilized in accordance with the present disclosure.

Naive Phage Library: Naive phage-displayed Helicon libraries were constructed using described methods. Briefly, the Peptide Display Cloning System kit from New England Biolabs was used to construct M13KE-based libraries (New England Biolabs, Ipswich, MA). Library oligonucleotides were chemically synthesized using a mix of trimer phosphoramides (Glen Research, Sterling, VA) without codons encoding cysteine, lysine, proline, or glycine, then annealed, extended, and ligated into a digested M13KE vector. All DNA products were purified using Monarch PCR and DNA cleanup kit (New England Biolabs, Ipswich, MA). The resulting library-containing phage vector was transformed into E. coli strain ER2738 (Lucigen, Middleton, WI) by electroporation and amplified by adding the post-rescue electroporated cells to a 500 mL E. coli culture at early-log phase (OD₆₀₀=0.01). Phage propagation, purification, and stapling were conducted as described.

### Trimerizer Library

Following identification of Helicon clusters specific for a presenter protein of interest based on screening the naive library, trimerizer library oligonucleotides were designed. Presenter-specific clusters of various sizes were used, ranging in size, e.g., from 10-mer to 20-mer. As an illustrative example of the design of a trimerizer library, a presenter-specific 20-mer cluster, X₁X₂X₃X₄W₅E₆C₇X₈E₉A₁₀A₁₁(F/I/L/M)₁₂X₁₃C₁₄X₁₅(F/Y)₁₆(F/Y)₁₇X₁₈X₁₉X₂₀ (SEQ ID NO: 23), is used. Briefly, codons of conserved or semi-conserved residues responsible for binding with a presenter protein are fixed or partially randomized in the primer to bias the library for retained affinity towards the chosen presenter protein (see, e.g., FIG. 18). The resulting primer (PR21) was generated as follows: for partial randomization (in parentheses), semi-degenerate codons are used to sample a subset of residues conserved within that position. In an example, position 12 is represented by four potential residues (F/L/I/M) from the identified presenter-specific cluster, so the semi-degenerate codon, WTK is used to code for phenylalanine (TTT), leucine (TTG), methionine (ATG), and isoleucine (ATT)-W represents A or T, and K represents G or T. For positions 16 and 17 within this example, the semi-degenerate codon, TWT, is used to code for both tyrosine (TAT) and phenylalanine (TTT) to represent all residues observed within the identified presenter-specific cluster. All other residues within the displayed Helicon with no apparent preference for presenter binding (represented as Xs within the cluster) are randomized to any other amino acid except cysteine, lysine, proline and glycine. Trimerizer libraries are built using multiple oligonucleotides from various designs based on the presenter specific binding sequences (see, e.g., Table E11-4). Library oligonucleotides are chemically synthesized using a mix of trimer phosphoramides (Glen Research, Sterling, VA) lacking codons for cysteine, lysine, proline, and glycine, annealed, extended, and ligated into a digested with KpnI and EagI restriction enzymes M13KE vector. The example PR21 oligonucleotide insert coding strand sequence is 5′-CATGCCCGGGTACCTTTCTATTCTCACTCTGCGCCGXXXXTGGGAATGTXGAAGCAGCAWTKXTGTXTWTTWTXXXGGTGGTTCTGGCGCAGGTCGTGGTTC-3′ (SEQ ID NO: 24), where X represents a single trimer phosphoramide incorporation, flanked by a KpnI restriction site. The antisense strand complements the 3′ end of the sense strand to allow Klenow extension, 5′-CATGTTTCGGCCGAACCACGACCTGCGCCAGAACCAC-3′ (SEQ ID NO: 22). The antisense strand possesses an EagI restriction site. Construction of the trimerizer library follows the previously described protocol for construction of the naive library.

Certain useful technologies are described in the Table below.

TABLE E11-4
Certain technologies.

| | | SEQ | |
|---|---|---|---|
| | | ID | |
| Name | Sequence | NO: | Presenter |
| PR01 | CATGCCCGGGTACCTTTCTATTCTCACTCTGCGCCGXXXXXXTGTXXg | 716 | CHIP |
| | cagcaASCATTTGTATGGTTGATXXXggtggttctggcgcaggtcgtggttc | | |
| PR02 | CATGCCCGGGTACCTTTCTATTCTCACTCTGCGCCGXXXXXXTGTXXg | 717 | CHIP |
| | cagcaASCATTTGTTGGATTGATXXXggtggttctggcgcaggtcgtggttc | | |
| PR03 | CATGCCCGGGTACCTTTCTATTCTCACTCTGCGCCGXXXXXXTGTXXg | 718 | CHIP |
| | cagcaGAGRTTTGTTGGMTTTATXXXggtggttctggcgcaggtcgtggttc | | |
| PR04 | CATGCCCGGGTACCTTTCTATTCTCACTCTGCGCCGXXXXXXTGTXCA | 719 | CHIP |
| | TgcagcaGATRTKTGTTGGMTTTWTXXXggtggttctggcgcaggtcgtggttc | | |
| PR05 | CATGCCCGGGTACCTTTCTATTCTCACTCTGCGCCGXXXTWTGAKXT | 720 | CHIP |
| | GTXXgcagcaATTAKGTGTMTTGTTXXXXggtggttctggcgcaggtcgtggttc | | |
| PR06 | CATGCCCGGGTACCTTTCTATTCTCACTCTGCGCCGXXXTTTTGGXTG | 721 | CHIP |
| | TCAGTWTgcagcaGCGAYGTGTXXXXXXggtggttctggcgcaggtcgtggttc | | |
| PR07 | CATGCCCGGGTACCTTTCTATTCTCACTCTGCGCCGXXXXTATXTGTA | 722 | CHIP |
| | TGGAGgcagcaSTGCTGTGTAYGXXXXXggtggttctggcgcaggtcgtggttc | | |
| PR08 | CATGCCCGGGTACCTTTCTATTCTCACTCTGCGCCGXXXXTGGAGCTG | 723 | CHIP |
| | TGYGGAGgcagcaTWTMTRTGTGAKXYWTXXXggtggttctggcgcaggtcgtggttc | | |
| PR09 | CATGCCCGGGTACCTTTCTATTCTCACTCTGCGCCGXXXASTATTTGG | 724 | CHIP |
| | TGTRTTGATgcagcaXXTGTXXXXXXggtggttctggcgcaggtcgtggttc | | |
| PR10 | CATGCCCGGGTACCTTTCTATTCTCACTCTGCGCCGXXXXXMTGTGT | 725 | CHIP |
| | ATTTGGgcagcaGATGATTGTXXXXXXggtggttctggcgcaggtcgtggttc | | |
| PR11 | CATGCCCGGGTACCTTTCTATTCTCACTCTGCGCCGXXXASCGATXTG | 726 | CHIP |
| | TGTGTATgcagcaTATTTCTGTGAKXXXXXggtggttctggcgcaggtcgtggttc | | |
| PR12 | CATGCCCGGGTACCTTTCTATTCTCACTCTGCGCCGXXXXXXTGTXXG | 727 | CHIP |
| | CGAGCMTTTGGTGTRTTGATXXXXggtggttctggcgcaggtcgtggttc | | |
| PR13 | CATGCCCGGGTACCTTTCTATTCTCACTCTGCGCCGXXXXXXTGTTGG | 728 | CHIP |
| | GAKGCGTGGVTTXTGTGAAXXXXXggtggttctggcgcaggtcgtggttc | | |
| PR14 | CATGCCCGGGTACCTTTCTATTCTCACTCTGCGCCGXXXXXXTGTTWT | 729 | MDM2 |
| | XGCAGCATTTXTGTVTTXGATXXXggtggttctggcgcaggtcgtggttc | | |
| PR15 | CATGCCCGGGTACCTTTCTATTCTCACTCTGCGCCGXXXXXGATTGTT | 730 | MDM2 |
| | TTXGCAGCATGGXTGTWTKXXXXXggtggttctggcgcaggtcgtggttc | | |
| PR16 | CATGCCCGGGTACCTTTCTATTCTCACTCTGCGCCGXXXSAAXXTGTT | 731 | MDM2 |
| | WTCAAGCAGCATGGXTGTVTTXGATXXXggtggttctggcgcaggtcgtggttc | | |
| PR17 | CATGCCCGGGTACCTTTCTATTCTCACTCTGCGCCGXXXXXXTGTTTT | 732 | MDM2 |
| | TGGGCAGCATGGXTGTXXXXXXggtggttctggcgcaggtcgtggttc | | |
| PR18 | CATGCCCGGGTACCTTTCTATTCTCACTCTGCGCCGXXXXXXTGTTTT | 733 | MDM2 |
| | CAGGCAGCATGGGATTGTCAGTWTTGGXXXggtggttctggcgcaggtcgtggttc | | |
| PR19 | CATGCCCGGGTACCTTTCTATTCTCACTCTGCGCCGXXXXXXTGTTTT | 734 | MDM2 |
| | CAGGCAGCATGGGATTGTCAGTWTTTTXXXggtggttctggcgcaggtcgtggttc | | |
| PR20 | CATGCCCGGGTACCTTTCTATTCTCACTCTGCGCCGXXXXXXTGTXGA | 735 | MDM2 |
| | TGCAGCATTTXTGTXTWTTATXXXggtggttctggcgcaggtcgtggttc | | |
| PR21 | CATGCCCGGGTACCTTTCTATTCTCACTCTGCGCCGXXXXTGGGAATG | 736 | MDM2 |
| | TXGAAGCAGCAWTKXTGTXTWTTWTXXXggtggttctggcgcaggtcgtggttc | | |
| PR22 | CATGCCCGGGTACCTTTCTATTCTCACTCTGCGCCGXXXXTGGXTGTT | 737 | MDM2 |
| | GGGAKGCAGCAXXTGTXGAKXXXXggtggttctggcgcaggtcgtggttc | | |
| PR23 | CATGCCCGGGTACCTTTCTATTCTCACTCTGCGCCGXXXXXXTGTXXG | 738 | MDM2 |
| | CAGCATTTXTGTXTGGXXXXggtggttctggcgcaggtcgtggttc | | |
| PR24 | CATGCCCGGGTACCTTTCTATTCTCACTCTGCGCCGXXXXXXTGTTWT | 729 | MDM2 |
| | XGCAGCATTTXTGTVTTXGATXXXggtggttctggcgcaggtcgtggttc | | |
| PR25 | CATGCCCGGGTACCTTTCTATTCTCACTCTGCGCCGXXXXTGGXTGTA | 739 | MDM2 |
| | CCASTGCAGCARTTXTGTXTWTXXXXggtggttctggcgcaggtcgtggttc | | |

| Mixed-base code | Mixed Bases |
|---|---|
| R | A, G |
| Y | C, T |
| M | A, C |
| K | G, T |
| S | G, C |
| W | A, T |
| H | A, C, T |
| B | G, C, T |
| V | A, C, G |
| D | A, G, T |
| N | A, C, G, T |

### Phage Library Screening.

### Naive Library Presenter Screening.

To conduct phage library screening, previously described procedures were utilized in accordance with the present disclosure. Briefly, Helicon-displayed phage libraries were incubated with streptavidin magnetic beads for 1 hr at room temperature in a buffer of 1×TBS, 1 mM MgCl₂, 1% (w/v) BSA, 0.1% Tween-20, 0.02% (w/v) sodium azide, 5% (w/v) nonfat milk. For each screening condition, 100 μL of 2 μM biotinylated protein was captured with streptavidin-coated magnetic beads (Dynabeads MyOne Streptavidin T1, ThermoFisher Scientific, Waltham, MA) that had been previously blocked with 1% BSA, 0.1% Tween-20, 2% glycerol in 1×TBS pH 7.4 at room temperature for 15 min, the supernatant was removed using a plate magnet and the beads are resuspended in 50 μL of the blocking buffer. 150 μL of the depleted phage library is added to each well for 200 μL final volume, plates are sealed, and the screening reactions are incubated at room temperature for 45 min, with rotation to maintain beads in solution. Following binding, beads were washed 5 times with ice-cold washing buffer (1×TBS, 1 mM MgCl2, 1% (w/v) BSA, 0.1% Tween-20, 0.02% (w/v) sodium azide, 2% (w/v) glycerol), beads containing protein-bound phage were collected and directly processed for NGS.

### Trimerizer Phage Screening.

Trimerizer phage screening is performed as for the naive library screening described above, with the key practical difference being that the Trimerizer library is incubated with a presenter protein after removal of the bead-binding phage library members and prior to the incubation with biotinylated proteins bound to streptavidin magnetic beads. To identify presenter-dependent phage-displayed Helicon members, target proteins were screened with the phage library in both the presence and absence of a presenter protein. Prior to addition of the bead-bound targets, sequences that bind to the target alone or to the presenter alone were removed: the phage library was split into two portions, and the presenter protein was added to one portion to a final concentration of 10 μM. 150 μL of the phage library without presenter protein was then added to a well containing 50 μL of the highest concentration of the target protein, and also to a blank (beads-only) well, both for a final volume of 200 μL. To the remaining wells, 150 μL of the phage library mixed with 10 μM presenter protein was added to wells containing 50 μL of the target of interest at a range of concentrations, and also to a blank (beads-only) well. The plate was sealed, and the screening reactions were incubated at room temperature for 45 min, with rotation to maintain beads in solution. The rest of the experiment was performed similar to the procedure described above with one exception—the addition of a presenter protein at a final concentration of 10 μM to the wash buffer (ice-cold iX TBS, 1 mM MgCl2, 1% (w/v) BSA, 0.1% Tween-20, 0.02% (w/v) sodium azide, 2% (w/v) glycerol).

### Next-Generation Sequencing.

Next-generation sequencing was performed as described in accordance with the present disclosure. Briefly, phage particles were denatured from magnetic beads at 95° C. for 15 min with an added spike-in sequence (a non-library member) that is used to enable cross-well normalization of sequence reads, followed by a two-step low-cycled PCR to introduce Illumina adaptors and 10 bp TruSeq DNA UD Indexes (Illumina, San Diego, CA) according to Illumina's 16S Metagenomic Sequencing Library Preparation protocol. The NGS library was sequenced with an Illumina NovaSeq platform using a 2x150 bp high-output kit (Illumina, San Diego, CA).

### Hit ID and Clustering.

Hit ID and Clustering is performed according to a described procedure in accordance with the present disclosure. Briefly, NGS reads were trimmed for quality (Phred score >18) and filtered for sequences that matched the design of the phage library. Counts for each unique sequence were tallied, and then normalized by the counts of the spike-in sequence added to each sample. A metric called Hit Strength was computed for each sequence as the fold-change between the normalized counts in the highest target concentration sample with presenter and the normalized counts in the target (no presenter) samples (averaged across experimental replicates). By using target wells with no presenter as “target blanks”, presenter-dependent binding could be identified. This approach eliminates sequences that show binding to target alone, or binding to a free presenter alone. When 0 counts are observed for a sequence in target only “target blank” samples, a count of 0.5 is used to prevent dividing by zero (Supplementary Dataset 1). In some embodiments, sequences with a hit strength greater than 5 were subjected to hierarchical clustering to identify sequence families.

Helicon synthesis.

Various technologies are available for preparing stapled peptides and can be utilized in accordance with the present disclosure. In some embodiments, technologies for synthesis of cysteine-stapled Helicons was utilized. Briefly, linear peptides containing two cysteine residues were synthesized at 100 or 250 μmol scale on Rink Amide resin (-0.5 mmol/g) using standard Fmoc-based solid phase peptide synthesis workflows. The peptides were globally deprotected and cleaved off-resin, then dissolved in DMSO. The DMSO stock was diluted in a 2:1 solvent mixture of acetonitrile and 50 mM ammonium hydroxide. The pH of the solution was adjusted to ˜8.5 using N, N-Diisopropylethylamine (DIPEA). For crosslinking of cysteine residues, ˜1.3 equivalents of the alkylating agent, N, N′-(1,4-phenylene)bis(2-bromoacetamide) in DMF were added to the crude peptide solution for two hours at room temperature. The crude helicons were purified by preparatory HPLC, and the purity of the final products were analyzed with analytical UPLC. The R8-S5 stapled peptides, including ATSP-7041 (P320), were synthesized according to reported procedures.

Certain agents and data are presented in Table E11-5 as examples.

TABLE E11-5
Certain trimerizer agents and data.
CHIP-PPIA C86

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PGPMRAECTVAATICMVDGAL | 740 | 9 | 108 | 334 | 272 | 300 | 0 | 9 | 0 | 0 |
| PLIALEDCTVAATICMVDGVV | 741 | 0 | 26 | 108 | 105 | 78 | 0 | 0 | 0 | 0 |
| PLQQEHLCSVAATICWIDGRI | 742 | 5 | 84 | 386 | 432 | 321 | 0 | 5 | 0 | 0 |
| PQQQIQYCTVAASICMVDGIT | 743 | 0 | 31 | 50 | 136 | 44 | 0 | 0 | 0 | 0 |
| PWDNEGFCTVAATICMVDGTV | 744 | 0 | 8 | 56 | 45 | 54 | 0 | 0 | 0 | 0 |
| PYSLQTACTVAATICWIDGTI | 745 | 0 | 17 | 134 | 166 | 78 | 0 | 0 | 0 | 0 |
| PYEEHTMCSVAATICWIDGPV | 746 | 0 | 22 | 204 | 174 | 163 | 0 | 0 | 0 | 3 |
| PKYFTADCSVAATICMVDGSS | 747 | 0 | 6 | 38 | 42 | 41 | 0 | 0 | 0 | 0 |
| PIEMMVECTAAATICMVDGWV | 748 | 0 | 0 | 42 | 39 | 34 | 0 | 0 | 0 | 0 |
| PMAEPEQCTVAATICMVDGMS | 749 | 0 | 0 | 58 | 49 | 75 | 0 | 0 | 0 | 0 |
| PMYMTGQCTVAATICMVDGTH | 750 | 0 | 1 | 71 | 73 | 78 | 0 | 0 | 0 | 0 |
| PYRDNVECGVAASICMVDGSS | 751 | 0 | 0 | 29 | 32 | 31 | 0 | 0 | 0 | 0 |
| PGPVYQHCTVAASICMVDGIT | 752 | 0 | 4 | 38 | 28 | 70 | 0 | 0 | 0 | 0 |
| PMATVQQCTVAATICWIDGII | 753 | 0 | 0 | 15 | 25 | 13 | 0 | 0 | 0 | 0 |
| PQYKINHCTVAASICMVDGRA | 754 | 0 | 3 | 23 | 36 | 28 | 0 | 0 | 0 | 0 |

CHIP-TEAD4 C87

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PVPFFWECQYAAAMCQQWYNK | 755 | 0 | 0 | 56 | 29 | 114 | 0 | 0 | 6 | 0 |
| PTPFFWDCQFAAAMCTKQPLR | 756 | 0 | 6 | 38 | 36 | 65 | 0 | 0 | 0 | 0 |
| PVPFFWECQYAAATCQTPRIK | 757 | 4 | 40 | 434 | 499 | 572 | 0 | 0 | 0 | 0 |
| PVPFFWECQYAAATCPLVTAK | 758 | 0 | 3 | 69 | 61 | 112 | 0 | 0 | 0 | 0 |
| PTNFYDPCVKAAIRCIVTDFE | 759 | 0 | 0 | 9 | 9 | 14 | 4 | 0 | 3 | 0 |
| PTPFFWECQFAAATCTAPRVQ | 760 | 13 | 244 | 1155 | 1428 | 1982 | 0 | 0 | 0 | 0 |
| PTPFFWDCQFAAATCYWSSHK | 761 | 3 | 2 | 15 | 22 | 19 | 0 | 0 | 0 | 0 |
| PVPFFWECQFAAATCIHVYPA | 762 | 0 | 0 | 50 | 60 | 90 | 0 | 0 | 0 | 0 |
| PVPFFWDCQFAAATCDAPQRR | 763 | 0 | 11 | 200 | 343 | 360 | 0 | 0 | 0 | 0 |
| PIPFFWECQFAAATCNRWTQW | 764 | 0) | 0 | 12 | 13 | 32 | 0 | 0 | 0 | 3 |
| PVPFFWPCQFAAATCAGTATQ | 765 | 0 | 0 | 18 | 15 | 68 | 0 | 0 | 0 | 0 |
| PTPFFWECQYAAAMCNWDRTH | 766 | 0 | 5 | 37 | 60 | 91 | 0 | 0 | 0 | 0 |
| PVPFFDECAQAAIRCIVTQVD | 767 | 0 | 0 | 8 | 7 | 32 | 0 | 0 | 0 | 0 |
| PMPFFWECQFAAATCDRRVNW | 768 | 0 | 0 | 11 | 29 | 25 | 0 | 0 | 0 | 0 |
| PVPFFWDCQYAAAMCVAPHRF | 769 | 0 | 0 | 17 | 44 | 40 | 0 | 0 | 0 | 0 |

CHIP-PIPA C88

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PNWMYWSCVEAAYLCENYVMV | 770 | 0 | 8 | 6 | 14 | 16 | 0 | 0 | 4 | 0 |
| PIDRHWSCVEAAYICENYVLT | 771 | 0 | 15 | 29 | 15 | 44 | 0 | 0 | 0 | 0 |
| PTMFSWSCVEAAYLCENYVLI | 772 | 0 | 18 | 42 | 17 | 62 | 0 | 0 | 0 | 0 |
| PKMQMWSCVEAAFLCENYVFI | 773 | 5 | 29 | 117 | 98 | 140 | 0 | 0 | 0 | 0 |
| PGYEMWSCAEAAFLCENYVWA | 774 | 5 | 40 | 171 | 95 | 191 | 0 | 0 | 0 | 3 |
| PHNGKWSCVEAAYICENYVYT | 775 | 0 | 26 | 384 | 286 | 328 | 0 | 0 | 0 | 0 |
| PAQDDWSCVEAAYLCENYVRV | 776 | 1 | 5 | 311 | 265 | 266 | 0 | 0 | 0 | 0 |
| PEHEQWSCVEAAYLCENYVMT | 777 | 3 | 41 | 294 | 293 | 375 | 0 | 0 | 0 | 0 |
| PHVSPWSCVEAAYLCENYVRT | 778 | 0 | 0 | 33 | 24 | 34 | 0 | 0 | 0 | 0 |
| PMQRQWSCVEAAYLCENYVMV | 779 | 0 | 13 | 111 | 115 | 147 | 0 | 0 | 0 | 0 |
| PPKSSWSCVEAAFICENYVRI | 780 | 0 | 8 | 73 | 72 | 106 | 0 | 0 | 0 | 0 |
| PIMETWSCVEAAYLCENYVKS | 781 | 0 | 0 | 175 | 204 | 196 | 0 | 0 | 0 | 0 |
| PHIQQWSCAEAAYMCENHVAT | 782 | 0 | 3 | 13 | 17 | 18 | 2 | 0 | 0 | 0 |
| PPYHSWSCVEAAYLCENYVRA | 783 | 0 | 1 | 48 | 69 | 80 | 0 | 0 | 0 | 0 |
| PPAPSWSCVEAAYLCENYVWV | 784 | 0 | 5 | 88 | 145 | 147 | 0 | 0 | 0 | 0 |

CHIP-PIPA C89

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PVRAYAMCGPAASICWIDGII | 785 | 3 | 39 | 219 | 197 | 235 | 0 | 0 | 0 | 0 |
| PILQGMACGPAATICWIDGII | 786 | 5 | 34 | 207 | 160 | 212 | 0 | 0 | 0 | 0 |
| PDMLAPMCGPAASICWIDGVI | 787 | 8 | 19 | 123 | 72 | 122 | 0 | 0 | 0 | 0 |
| PPFHMMMCGPAASICMVDGVV | 788 | 0 | 13 | 240 | 212 | 222 | 2 | 0 | 0 | 0 |
| PDMEFITCGPAASICMVDGMT | 789 | 0 | 0 | 17 | 15 | 34 | 0 | 5 | 0 | 0 |
| PTINMNNCGPAASICWIDGVV | 790 | 0 | 4 | 50 | 62 | 122 | 0 | 0 | 0 | 0 |
| PTSEFVVCGAAASICWIDGKV | 791 | 0 | 0 | 6 | 10 | 16 | 0 | 0 | 0 | 0 |
| PMYMTDACGPAATICMVDGVV | 792 | 0 | 8 | 104 | 236 | 199 | 0 | 0 | 0 | 0 |
| PYPWMHDCGVAATICWIDGVI | 793 | 2 | 10 | 60 | 136 | 127 | 0 | 1 | 0 | 3 |
| PDHRFVYCGPAATICMVDGSV | 794 | 0 | 3 | 19 | 84 | 59 | 0 | 0 | 0 | 0 |
| PHPVDYMCGPAATICMVDGYI | 795 | 0 | 2 | 6 | 29 | 57 | 0 | 0 | 0 | 0 |
| PMSYTKACGPAASICWIDGFI | 796 | 3 | 0 | 0 | 20 | 21 | 0 | 0 | 0 | 0 |
| PWGRPVECGPAATICMVDGKI | 797 | 18 | 402 | 5468 | 6954 | 6280 | 0 | 6 | 0 | 26 |
| PDPVHVMCGPAASICMVDGVV | 798 | 14 | 189 | 1028 | 1016 | 667 | 0 | 0 | 13 | 0 |
| PMWQWPECGPAATICWIDGMI | 799 | 0 | 27 | 159 | 135 | 80 | 0 | 0 | 0 | 0 |

CHIP-TEAD4 C90

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PDDPMDLCEYAAEVCWIYIDS | 800 | 0 | 26 | 45 | 60 | 35 | 0 | 0 | 0 | 0 |
| PQEGVDLCEYAAEVCWIYIKE | 801 | 0 | 0 | 13 | 25 | 16 | 0 | 0 | 0 | 0 |
| PDLPVDICAHAADVCWLYLDH | 802 | 0 | 6 | 74 | 121 | 198 | 0 | 0 | 0 | 0 |
| PPWPVDICQHAAEVCWLYLPI | 803 | 0 | 0 | 5 | 12 | 16 | 0 | 0 | 0 | 0 |
| PYEPFWLCQFAAATCHRWMEV | 804 | 0 | 0 | 2 | 7 | 9 | 6 | 2 | 0 | 0 |
| PIEYIDVCDYAAEICWIYAES | 805 | 0 | 0 | 6 | 28 | 56 | 0 | 0 | 0 | 0 |
| PLHPIDVCEYAAEVCWIYLDD | 806 | 0 | 0 | 1 | 9 | 12 | 0 | 0 | 0 | 0 |
| PDEPIDLCEHAAEICWLYDQS | 807 | 0 | 0 | 3 | 12 | 83 | 0 | 0 | 0 | 0 |
| PQWPVDMCNHAADVCWIYMDY | 808 | 4 | 0 | 1 | 19 | 21 | 0 | 0 | 3 | 0 |
| PYEEVDLCEHAADVCWLYLDV | 809 | 0 | 0 | 0 | 15 | 19 | 0 | 0 | 0 | 0 |
| PEPGPDICAHAADVCWIYVEE | 810 | 0 | 0 | 0 | 13 | 12 | 0 | 0 | 0 | 0 |
| PHSFIDLCEHAADVCWLYGDD | 811 | 0 | 10 | 0 | 31 | 44 | 0 | 2 | 0 | 0 |
| PVMPVWSCVEAAYMCEWLLDP | 812 | 0 | 5 | 0 | 7 | 18 | 0 | 0 | 0 | 0 |

MDM2-beta-catenin C91

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PPVKEAICFQAAWQCLSDDWA | 813 | 0 | 22 | 26 | 18 | 51 | 0 | 0 | 0 | 0 |
| PIEMQSVCFQAAWTCLSDTWY | 814 | 5 | 31 | 28 | 62 | 46 | 0 | 0 | 0 | 0 |
| PIYHTNDCFGAAWYCFSEEWV | 815 | 8 | 22 | 28 | 42 | 37 | 0 | 0 | 0 | 1 |
| PFKDERVCFQAAWMCVSDDYN | 816 | 12 | 31 | 44 | 74 | 46 | 0 | 0 | 0 | 0 |
| PTNIEQPCFQAAWQCVTDDWT | 817 | 0 | 11 | 17 | 21 | 20 | 0 | 0 | 0 | 0 |
| PMEQQAICFQAAWMCLADDWT | 818 | 17 | 58 | 121 | 124 | 92 | 0 | 0 | 0 | 0 |
| PWKYEQVCYQAAWQCLSDDWD | 819 | 40 | 105 | 179 | 209 | 287 | 0 | 0 | 3 | 0 |
| PNLPQRVCFQAAWLCLSDDWT | 820 | 0 | 6 | 24 | 29 | 15 | 0 | 0 | 0 | 0 |
| PRNSENICYQAAWYCLTDDWI | 821 | 0 | 6 | 29 | 19 | 41 | 0 | 0 | 0 | 0 |
| PGWKQGICFQAAWMCVADEWI | 822 | 3 | 10 | 30 | 35 | 36 | 0 | 0 | 0 | 0 |
| PFMSQEVCFQAAWLCVMDDYV | 823 | 4 | 11 | 25 | 33 | 35 | 0 | 0 | 0 | 0 |
| PIKTEHVCYQAAWQCLVDDEN | 824 | 2 | 4 | 29 | 14 | 46 | 0 | 0 | 2 | 0 |
| PYVHQDICFQAAWMCVKDEWM | 825 | 10 | 17 | 42 | 60 | 51 | 0 | 0 | 0 | 0 |

MDM2-beta-catenin C92

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PTQFKPDCFGAAFTCIHDMYM | 827 | 6 | 5 | 5 | 6 | 7 | 0 | 0 | 0 | 0 |
| PDQHHHDCFGAAWHCLLFLED | 828 | 0 | 10 | 11 | 5 | 24 | 0 | 0 | 0 | 0 |
| PAKYQQDCFRAAWGCLEYLYY | 829 | 9 | 86 | 99 | 134 | 162 | 0 | 0 | 0 | 0 |
| PEQFQKDCFPAAWQCLVYIYG | 830 | 0 | 5 | 7 | 13 | 7 | 0 | 0 | 0 | 0 |
| PISAANDCFKAAWQCIIWLHQ | 831 | 12 | 20 | 69 | 58 | 62 | 0 | 0 | 0 | 0 |
| PSENARDCFWAAWDCLYFIYQ | 832 | 8 | 17 | 63 | 58 | 76 | 0 | 4 | 0 | 0 |
| PEPYIADCFDAAWRCLMYIYD | 833 | 0 | 4 | 10 | 9 | 18 | 0 | 0 | 7 | 0 |
| PKVAPPDCFEAAWNCLYFIYN | 834 | 0 | 0 | 9 | 6 | 9 | 0 | 0 | 0 | 0 |
| PHQDKQDCFGAAWTCIEWIYQ | 835 | 0 | 37 | 89 | 94 | 167 | 0 | 0 | 0 | 0 |
| PVQDHPDCFFAAWDCLDWLYT | 836 | 0 | 0 | 10 | 7 | 22 | 0 | 10 | 0 | 0 |
| PSRAKDDCFKAAWNCLEYIWH | 837 | 0 | 3 | 10 | 27 | 13 | 0 | 0 | 0 | 0 |
| PEERKNDCFDAAWECIQYLYD | 838 | 7 | 24 | 108 | 226 | 176 | 0 | 0 | 0 | 2 |
| PDQNEEDCYQAAWTCILDLYR | 839 | 0 | 0 | 4 | 8 | 8 | 0 | 0 | 0 | 4 |
| PEEWRGDCFSAAWDCLNYIYD | 840 | 0 | 0 | 7 | 16 | 11 | 0 | 0 | 0 | 0 |

MDM2-beta-catenin C93

| | SEQ ID | Presenter + Target | | | | | | | TARGET | TARGET |
|---|---|---|---|---|---|---|---|---|---|---|
| Sequence | NO: | 30 | 100 | 300 | 1000 | BLANK | BLANK | BLANK | BLANK | BLANK |
| PNLTGADCFPAAWQCLQFLWD | 841 | 83 | 128 | 146 | 142 | 126 | 0 | 0 | 0 | 0 |
| PKGLQSICYQAAWQCLVDTWD | 842 | 30 | 56 | 111 | 137 | 101 | 0 | 0 | 0 | 3 |
| PAWEQMYCFQAAWQCITDIWD | 843 | 3 | 4 | 11 | 9 | 22 | 0 | 0 | 0 | 0 |
| PQITKADCFPAAWECIMVIFD | 844 | 0 | 0 | 6 | 5 | 8 | 0 | 0 | 0 | 0 |
| PDTQQNTCFQAAWMCVSDDWD | 845 | 0 | 10 | 27 | 27 | 51 | 0 | 0 | 0 | 0 |
| PITQESVCFQAAWQCLKDPWE | 846 | 0 | 0 | 5 | 5 | 7 | 0 | 0 | 0 | 0 |
| PQWEESVCFQAAWHCLADTWD | 847 | 0 | 0 | 10 | 25 | 43 | 0 | 3 | 0 | 0 |
| PLLSQYPCFQAAWQCISDIFD | 848 | 0 | 3 | 3 | 6 | 24 | 0 | 0 | 0 | 0 |
| PDPQETWCFQAAWQCVADTWD | 849 | 0 | 0 | 3 | 12 | 10 | 4 | 0 | 0 | 0 |
| PMTTQQVCFQAAWQCVGDTWD | 850 | 0 | 0 | 1 | 16 | 20 | 0 | 0 | 0 | 0 |
| PEIKEWACFQAAWQCLDDTWD | 851 | 0 | 0 | 0 | 6 | 16 | 0 | 0 | 0 | 0 |
| PKIIQSVCFQAAWQCLTDTYF | 852 | 0 | 0 | 0 | 6 | 14 | 0 | 0 | 0 | 0 |
| PKTMQPVCFQAAWMCVTDHWD | 853 | 4 | 0 | 3 | 14 | 17 | 0 | 3 | 0 | 0 |
| PKLNQQECFQAAWWCVRDPWD | 854 | 0 | 3 | 0 | 13 | 6 | 0 | 0 | 0 | 0 |

CHIP-PPIA C94

| | SEQ ID | Presenter +Target | | | | | | | TARGET | TARGET |
|---|---|---|---|---|---|---|---|---|---|---|
| Sequence | NO: | 30 | 100 | 300 | BLANK | BLANK | BLANK | BLANK | BLANK | BLANK |
| PPRHENECIHAADICWLYWLY | 855 | 0 | 6 | 86 | 41 | 171 | 0 | 0 | 0 | 0 |
| PYSIPTMCQHAADICWLYWLY | 856 | 0 | 6 | 33 | 51 | 28 | 0 | 0 | 0 | 0 |
| PLMLDQECTDAAEVCWIYWLY | 857 | 0 | 5 | 33 | 44 | 31 | 0 | 0 | 0 | 0 |
| PLWDDQYCDHAADVCWLYWLY | 858 | 0 | 8 | 42 | 60 | 41 | 0 | 3 | 0 | 0 |
| PDWQATHCPHAADVCWLYWLY | 859 | 0 | 8 | 27 | 46 | 28 | 0 | 0 | 0 | 0 |
| PDVVEVPCLNAAEVCWLYWLY | 860 | 0 | 12 | 38 | 48 | 62 | 0 | 0 | 0 | 0 |
| PWGSTDICVYAAYFCESEGLY | 861 | 0 | 0 | 8 | 8 | 13 | 0 | 0 | 0 | 0 |
| PLEQFQQCVDAAEVCWLYWLY | 862 | 0 | 0 | 10 | 11 | 16 | 0 | 0 | 0 | 0 |
| PGPQQDKCAQAAEICWLYWLY | 863 | 0 | 2 | 15 | 10 | 41 | 0 | 0 | 0 | 0 |
| PETEYPQCQDAAEVCWLYWVY | 864 | 0 | 0 | 19 | 29 | 21 | 0 | 0 | 0 | 0 |
| PKLEPSNCNHAADVCWLYWLY | 865 | 0 | 21 | 142 | 212 | 321 | 0 | 0 | 0 | 0 |
| PEPEYSACSNAAEICWLYWVY | 866 | 0 | 0 | 10 | 14 | 26 | 0 | 0 | 0 | 0 |
| PIADTQLCVEAAEICWLYWLY | 867 | 0 | 0 | 15 | 29 | 31 | 0 | 0 | 0 | 0 |
| PTPSSGQCPHAADVCWLYWLY | 868 | 6 | 31 | 163 | 271 | 434 | 0 | 0 | 0 | 0 |
| PAQVQHNCPDAAEICWLYWLH | 869 | 0 | 0 | 15 | 32 | 28 | 0 | 0 | 0 | 0 |

Blank = Media only.
Target Blank = target only (no presenter).
Unless otherwise noted, target and presenter concentrations in nM.

### Surface Plasmon Resonance (SPR) Spectroscopy.

SPR of E3-binding helicons: To confirm Helicon binding to all selected E3 ligases and E3-related proteins, SPR experiments were performed on a Biacore 8K (Cytiva) instrument at 25° C. in 1×HBS-P+ buffer (Cytiva) with 1% DMSO. A SA Series S sensor chip was docked and pre-conditioned with three injections of 50 mM NaOH/1 M NaCl to remove unbound streptavidin from the surface. Biotinylated proteins, including CUL4B^{NTD}, CUL5^{NTD}, WWP1^{WW-HECT}, WWP1^{HECT}, WWP2^{HECT}, VHL-ELOBC, SOCS2-ELOBC, CHIP²³⁻¹⁵⁴, CHIP²³⁻³⁰³, and MDM2²⁵⁻¹⁰⁹, were each diluted to 5-10 μg/mL in running buffer and immobilized to channels 1 through 8 at 5 μL/min for 50-80 sec for a final immobilization level of ˜500-2000 R U. Helicons were diluted to 5 μM in running buffer and then serially diluted 2-fold for a total of seven concentrations with one blank (7-point two-fold Helicon dilution series with top concentration=5 μM and bottom concentration=78 nM). Compounds were injected over the immobilized and reference surfaces at 30 μL/min for 60 sec and then allowed to dissociate for 180 sec without surface regeneration (N=1-2). Data were analyzed using Biacore Insight Evaluation Software (Cytiva). Sensorgrams were double referenced, with most of them fitted to a 1:1 steady-state affinity model, with a few fitted with both the steady-state affinity model and the kinetics model.

SPR analysis of the trimerizer Helicon-dependent ß-catenin:MDM2 interaction: To probe the trimerizer Helicon-dependent CTNNB1: MDM2 interaction, SPR experiments were performed on a Biacore S200 (Cytiva) instrument at 25° C. in 1×HBS-P+ buffer (Cytiva) with 1% DMSO. CTNNB1¹³⁴⁻⁶⁶⁵ was immobilized using the Biotin CAPture Kit, Series S (Cytiva) to ˜600-1000 RU. Tag-free MDM2¹⁷⁻¹¹¹ was diluted to 625 nM then serially diluted 2-fold for a total of seven concentrations with one blank (7-point two-fold Helicon dilution series with top concentration=625 nM and bottom concentration=9.8 nM), in running buffer in the absence or presence of 1 μM trimerizer Helicon (n=3). MDM2-binding Helicons were injected over the immobilized and reference surfaces at 30 μL/min for 90 sec and then allowed to dissociate for 270 sec. Surface was regenerated with a 120-second injection of CAP regeneration solution each cycle. Data was analyzed using Biacore Insight Evaluation software (Cytiva). Sensorgrams were double-referenced and evaluated for competition.

SPR analysis of trimerizer Helicons against ß-catenin: To understand how the trimerizer Helicons interact with CTNNB1 by themselves, SPR experiments were performed on a Biacore S200 (Cytiva) instrument at 25° C. in 1×HBS-P+ buffer (Cytiva) with 1% DMSO. CTNNB1³⁴⁻⁶⁶⁵ was immobilized using the Biotin CAPture Kit, Series S (Cytiva) to -600-1000 RU, while Helicons were diluted to 10 μM in running buffer then serially diluted 2-fold for a total of four concentrations with one blank (4-point two-fold Helicon dilution series with top concentration=10 μM and bottom concentration=1.25 μM). Data was analyzed using Biacore Insight Evaluation software (Cytiva).

SPR competition, ABA mode: CUL5: SOCS2-ELOBC. To probe the H314-binding site on CUL5, SPR ABA experiments were performed on a Biacore S200 (Cytiva) instrument at 25° C. in 1×HBS-P+ buffer (Cytiva) with 1% DMSO. Biotinylated CUL5 (residues 1-186) was immobilized using the Biotin CAPture Kit, Series S (Cytiva) to -150 RU. For each injection, H314 in 10 mM DMSO stock was diluted to 10 μM in SPR running buffer and was injected over the surface for 120 sec at 30 μL/min to achieve equilibrium binding. 100 nM tag-free SOCS2-ELOBC was then injected for 60 sec at 30 μL/min in the absence or presence of H314 over the surface. Surface was regenerated with a 120-second injection of CAP regeneration solution each cycle. Data was analyzed using Biacore Insight Evaluation software (Cytiva). Sensorgrams were double-referenced and evaluated for competition.

SPR, ABA mode: TEAD4: CHIP. To confirm the ternary complex formation of TEAD4: CHIP, SPR ABA experiments were performed on a Biacore S200 (Cytiva) instrument at 25° C. in 1×HBS-P+ buffer (Cytiva) with 1% DMSO. Biotinylated CHIP²³-3⁰³ was immobilized using the Biotin CAPture Kit, Series S (Cytiva) to -200 RU. For each injection, Helicons in 10 mM DMSO stock were diluted to 10 M in SPR running buffer and were injected over the surface for 120 sec at 30 μL/min to achieve equilibrium binding (A). 300 nM tag-free TEAD4²¹⁷⁴³⁴ protein was then injected for 60 sec at 30 μL/min (B) in the absence or presence of Helicons over the surface. The surface was regenerated with a 120-second injection of CAP regeneration solution each cycle. Data was analyzed using Biacore Insight Evaluation software (Cytiva). Sensorgrams were double-referenced and evaluated for competition.

SPR, ABA mode: MDM2:0-catenin. To confirm the ternary complex formation of MDM2:β-catenin, SPR ABA experiments were performed on a Biacore S200 (Cytiva) instrument at 25° C. in 1×HBS-P+ buffer (Cytiva) with 1% DMSO. Biotinylated MDM2²⁵⁻¹⁰⁹ was immobilized using the Biotin CAPture Kit, Series S (Cytiva) to ˜200 RU. For each injection, Helicons in 10 mM DMSO stock were diluted to 10 M in SPR running buffer and were injected over the surface for 120 sec at 30 μL/min to achieve equilibrium binding. 150 nM tag-free CTNNB1³⁴⁻⁶⁶⁵ protein was then injected for 60 sec at 30 μL/min in the absence or presence of Helicons over the surface. Surface was regenerated with a 120-second injection of CAP regeneration solution each cycle. Data was analyzed using Biacore Insight Evaluation software (Cytiva). Sensorgrams were double-referenced and evaluated for competition.

Auto-ubiquitylation of WWP2 (ELISA). E3LITE Customizable Ubiquitin Ligase Kit (LifeSensors, UC101) was used to assess the autoubiquitination activity of HECT domain of WWP2 (WWP2^{HECT}). The ELISAs were performed with steps following the manufacturer protocol, with all solutions freshly made before the start of the experiment and protein components carefully stored on ice before addition (n=3). The concentration of the catalytic HECT domain was 50 nM. The Helicon inhibitors were used at a concentration of 10 μM, with DMSO as the negative control. Relative Luminescence Units (RLUs) were recorded with a GloMax™ Discover luminometer.

Assessment of ternary complex formation with Time-Resolved Fluorescence Energy Transfer (TR-FRET) and Fluorescence Polarization (FP).

TR-FRET analysis of the TEAD4: CHIP complex. For the TR-FRET ternary complex formation of the TEAD4: CHIP pair, biotinylated recombinant TEAD4²¹⁷⁻⁴³⁴ was diluted to 100 nM, Alexa Fluor™ 488 labeled CHIP²³⁻³⁰³ was diluted to 150 nM and Terbium-labeled streptavidin (Cis-Bio) was diluted to 2 nM in assay buffer (10 mM HEPES pH 7.5, 150 mM NaCl, 0.05% Tween-20) in a final volume of 20 μL per well of a black 384-well plate (Costar). Compounds were serially diluted in 90% DMSO and 40 nL of compound (11-point three-fold Helicon dilution series with top concentration=20 μM) was added to the plate and the samples were incubated for 60 mins at room temperature (n=2). FRET signal was determined using a LanthaScreen™ filter on a PheraStar (BMG Biotech) plate reader (Ex: 337 nm; Emi: 490 nM; Em₂: 520 nM). The ratio of Em₅₂₀ to Em₄₉₀ was calculated and plotted against compound concentration. Resulting data was fit to a four-parameter dose-response curve with variable slope. The positive control chimeric compound, P325, Ac-LWWPDGSGSGGSPGOVPMRKROLPASFWEEPR-NH₂ (SEQ ID NO: 42), is a designed bi-functional molecule with its N-terminus adapted from the CHIPopt peptide that interacts with CHIP (Ravalin, M. et al. Specificity for latent C termini links the E3 ubiquitin ligase CHIP to caspases. Nat Chem Biol 15, 786-794 (2019)), and the C-terminus derived from the FAM181A fragment that interacts with TEAD4 (Bokhovchuk, F. et al. Identification of FAM181A and FAM181B as new interactors with the TEAD transcription factors. Protein Sci 29, 509-520 (2020)). The curve for the positive control was fitted with a Biphasic Curve function in Prism 9 (GraphPad).

TR-FRET analysis of the PPIA: CHIP complex. For the TR-FRET ternary complex formation of the PPIA: CHIP pair, biotinylated recombinant full length PPIA was diluted to 100 nM, Alexa Fluor™ 488 labeled CHIP²³⁻³⁰³ was diluted to 150 nM and Terbium-labeled streptavidin (Cis-Bio) was diluted to 2.3 nM in assay buffer (10 mM HEPES pH 7.5, 150 mM NaCl, 0.05% Tween-20) in a final volume of 20 L per well of a black 384-well plate (Costar). Compounds were serially diluted in 90% DMSO, and 40 nL of compound (11-point three-fold Helicon dilution series with top concentration=20 μM) was added to the plate. To probe the trimerizer interface of PPIA, CHIP, 2 μM CHIP-binding Helicon H318 or 2 μM cyclosporine A (CsA) was added to the assay buffer. Assay plates (n=2) were incubated for 60 mins at room temperature. FRET signal was determined using a LanthaScreen™ filter on a PheraStar (BMG Biotech) plate reader (Ex: 337 nm; Em₁: 490 nM; Em₂: 520 nM). The ratio of Em₅₂₀ to Em₄₉₀ was calculated and plotted against compound concentration. The resulting data was fitted to a four-parameter dose-response curve with variable slope.

FP analysis of the β-catenin: MDM2 and β-catenin:MDM4 complexes: For the FP analysis of β-catenin ternary complex formation, compounds at 10 mM in DMSO were serially diluted 1:3 in DMSO for a total of 11 concentrations using a Mosquito LV (SPT Labtech), then diluted 1000-fold in buffer (50 mM HEPES, pH 7.5, 125 mM NaCl, 2% glycerol, 0.5 mM EDTA, 0.05% v/v pluronic acid) in duplicate by the Mosquito LV (SPT Labtech) into a black polystyrene 384-well plate (Corning) (11-point three-fold Helicon dilution series with top concentration=10 μM). Protein-probe solution includes 80 nM full-length β-catenin or 130 nM β-catenin residues 134-665, mixed with 25 nM MDM2¹⁷⁻¹¹¹ or MDM4¹⁴⁻¹¹¹ labeled with Alexa Fluor™ 488. Protein-probe solution was plated into the Helicon plate using the MultiDrop Combi (Thermo Fisher) for a total reaction pool of 40 μL. The plate was incubated and protected from light for 1 hr at room temperature prior to reading. (n=2) reads were performed on a CLARIOstar plate reader (BMG Labtech) with excitation at 485 nm, emission at 525 nm, and cutoff at 504 nm, and the resulting data was fit to a four-parameter dose-response curve with variable slope. To probe the trimerizer interface of MDM2:0-catenin, 2 μM ALRN-6924 peptide¹¹ or 100-400 nM ICAT (residues 1-81) recombinant proteins were added to the assay buffer, and the plates were prepared similarly. Positive control Helicon, P335, Ac-PD-cyclopentylalanine-CDDAAFNC-3Thi-benzothienylalanine-QGSGS-bAla-LTFEHYWAQLTS-NH₂ (SEQ ID NO: 43) (Cys-stapled), is a designed bi-functional molecule comprising at its N-terminus a β-catenin-interacting Helicon¹⁹ and at its C-terminus, a p53-derived peptide that interacts with MDM2 (Czarna, A. et al. High affinity interaction of the p53 peptide-analogue with human Mdm2 and Mdmx. Cell Cycle 8, 1176-1184 (2009)). The curve for the positive control was fitted with the Biphasic Curve function in Prism 9.

### Competition Fluorescence Polarization.

Competition FP of TEAD4: For the competition FP of TEAD4, Helicons at 10 mM in DMSO were serially diluted 1:3 in DMSO for a total of 11 concentrations using a Mosquito LV (SPT Labtech), then diluted 1000-fold in buffer (50 mM HEPES, pH 7.5, 150 mM NaCl, 0.5 mM TCEP, 0.05% v/v pluronic acid) in duplicate by the Mosquito LV (SPT Labtech) into a black polystyrene 384-well plate (Corning) (11-point three-fold Helicon dilution series with top concentration=10 μM). Probe solution (40 nM TEAD4, mixed with 10 nM 5FAM-labeled YAP1 residues 10-53, Uniprot ID: Q9NQB0, in buffer) with or without 10 μM CHIP²³⁻³⁰³ recombinant protein as the presenter was prepared and plated using the MultiDrop Combi (Thermo Fisher) for a total reaction pool of 40 μL. The plate was incubated and protected from light for 1 hr at room temperature prior to reading. Reads were performed on a CLARIOstar plate reader (BMG Labtech) with excitation at 485 nm, emission at 525 nm, and cutoff at 504 nm. Data were fitted to a 1:1 binding model with Hill slope using an in-house script.

Competition FP of CHIP: For the competition FP of CHIP, the assay was performed similarly to the competition FP of TEAD4. The assay was performed in buffer: 1×HBS-P+(Cytiva), with 400 nM CHIP²³-3⁰³ recombinant protein as target, and 20 nM CHIP-binding peptide as probe (5FAM-bAla-SSGPTIEEVD (SEQ ID NO: 44), derived from HSP70 (Smith, M. C. et al. The E3 Ubiquitin Ligase CHIP and the Molecular Chaperone Hsc70 Form a Dynamic, Tethered Complex. Biochemistry-us 52, 5354-5364 (2013)). The plates were incubated and protected from light for 1 hr at room temperature, and resulting data were fitted to a 1:1 binding model with Hill slope using an in-house script.

Competition FP of MDM2: For the competition FP of MDM2, the assay was performed similarly to the competition FP of TEAD4. The assay was performed in buffer: lx PBS with 0.01% Tween, with 30 nM MDM2¹⁷-1 “recombinant protein as target, and 3 nM MDM2-binding peptide as probe (5FAM-bAla-LTFEHYWAQLTS-NH₂ (SEQ ID NO: 45), derived from p53). The plates were incubated and protected from light for 1 hr at room temperature, and resulting data were fitted to a 1:1 binding model with Hill slope using an in-house script.

Competition FP of VHL: For the competition FP of VHL, the assay was performed similarly to the competition FP of TEAD4. The assay was performed in the buffer: 10 mM HEPES, pH 7.5, 50 mM NaCl, 0.05% v/v pluronic acid, 15 nM VHL-ELOBC recombinant protein, and 5 nM VHL tracer, HXC78 (Han, X. et al. Discovery of ARD-69 as a Highly Potent Proteolysis Targeting Chimera (PROTAC) Degrader of Androgen Receptor (AR) for the Treatment of Prostate Cancer. J Med Chem 62, 941-964 (2019)). A structurally similar small molecule VHL binder, VH298 (Sigma SML1896) (Frost, J. et al. Potent and selective chemical probe of hypoxic signalling downstream of HIF-α hydroxylation via VHL inhibition. Nat Commun 7, 13312 (2016)) was included in the competition FP assay as a positive control. The plates were incubated and protected from light for 1 hr at room temperature, and resulting data were fitted to a 1:1 binding model with Hill slope using an in-house script. HXC78 systematic name: (2S,4R)-N-((S)-1-(3′,6′-dihydroxy-3-oxo-3H-spiro[isobenzofuran-1,9′-xanthen]-5-yl)-17-(4-(4-methylthiazol-5-yl)phenyl)-1,15-dioxo-5,8,11-trioxa-2,14-diazaheptadecan-17-yl)-4-hydroxy-1-((R)-3-methyl-2-(3-methylisoxazol-5-yl)butanoyl)pyrrolidine-2-carboxamide; VHL298 systematic name: (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide.

### Crystal Structure Data Collection.

To obtain the structures of the protein-Helicon complexes, briefly, 10 mM of Helicon stock in 90% DMSO were added to the protein stocks to a final 1:1.25 protein: Helicon molar ratio or 1:1:1.25 protein-A: protein-B: Helicon and screened against commercially available crystallization screens. Crystals were obtained by hanging or sitting hanging drop vapor diffusion methods at room temperature, with their crystallization conditions detailed in, e.g., Table E11-6. Crystals were cryo-protected with glycerol or ethylene glycol, followed by flash-freezing in liquid nitrogen. Diffraction datasets were collected at 100K. Data were processed in XDS & XSCALE, AIMLESS, and/or STARANISO, all parts of the autoPROC suite. Molecular replacement solutions were obtained using PHASER with previously deposited high-resolution PDB structures as search models. Complete models were built through iterative cycles of manual model building in COOT and structure refinement was carried out using either REFMAC or PHENIX. All the structure model figures in the paper were prepared using PyMOL (The PyMOL Molecular Graphics System, Version 2.4, Schrödinger, LLC.). The atomic coordinates and structure factors have been deposited in the Protein Data Bank. Certain technologies are presented below as examples.

Certain observed interactions are described below (Residues involves in Interaction (with 4.5A)).

β-catenin_MDM2_Helicon332 (PDB code: 8EI9). beta-Catenin by H332: Glu620, Thr653, Tyr654, Ala656, Ala657 and Phe660. MDM2 by H332: Met50, Lys51, Leu54, Phe55, Leu57, Gly58, Ile61, Met62, Tyr67, Gln72, Val75, Val93, Lys94, His96, Ile99, Tyr100 and Tyr104. beta-Catenin by MDM2: Arg582, Val584, Arg587, Cys619, Gln623, Ala656, Ala657, Phe660, Glu664 and Asp665. MDM2 by beta-catenin: Gln71, His73, Val93, Lys94, Glu95, His96 and Arg97.

β-catenin_MDM2_Helicon333 (PDB code: 8EIA). beta-Catenin by H333: Arg582, Glu620, Thr653, Tyr654, Ala656, Ala657 and Phe660. MDM2 by H333: Met50, Lys51, Leu54, Leu57, Gly58, Ile61, Gln72, Val75, Val93, Lys94, His96 and Tyr100. beta-Catenin by MDM2: Arg582, Val584, Arg587, Cys619, Gln623, Ala656, Ala657, Val658, Phe660 and Arg661. MDM2 by beta-catenin: His73, Val93, Lys94, Glu95, His96 and Arg97.

β-catenin_MDM2_Helicon329 (PDB code: 8EIB). beta-Catenin by H329: Arg474, His475, Arg515, Leu519, His578, Arg582, Arg612, Glu620, Gln623, Gly650, Thr653, Tyr654, Ala656, Ala657 and Phe660. MDM2 by H329: Thr26, Met50, Leu54, Leu57, Gly58, Ile61, Met62, Tyr67, Gln72, His73, Val75, Val93, Lys94, His96, Ile99, Tyr100 and Tyr104. beta-Catenin by MDM2: Tyr432, Arg474, His475, Gln476, Ala478, Glu479, Arg582 and Glu649. MDM2 by beta-catenin: Glu25, Lys51, Phe55, His96, Arg97, Tyr104, Arg105 and Val109.

β-catenin_MDM2_Helicon330 (PDB code: 8EIC). beta-Catenin by H330: Tyr432, Arg474, His475, Arg515, Leu519, His578, Arg582, Arg612, Cys619, Glu620, Gln623, Gly650, Thr653, Tyr654, Ala656, Ala657, Phe660 and Arg661. MDM2 by H330: Thr26, Met50, Leu54, Leu57, Gly58, Ile61, Met62, Tyr67, Gln72, His73, Val75, Val93, Lys94, His96, Ile99, Tyr100 and Tyr104. beta-Catenin by MDM2: Asn430, Tyr432, Lys433, Arg474, His475, Gln476, Glu479 and Arg582. MDM2 by beta-catenin: Glu25, Thr26, His96, Arg97, Tyr104, Val109 and Val110.

CHIP_Helicon317 (PDB code: 8EHZ). CHIP by Helicon317: LYS30, ASN34, PHE37, VAL38, TYR49, VAL61, ASN65, LEU68, LEU71, LYS72, LYS95, PHE98, PHE99, GLN102, GLN127, LEU129, ASN130, PHE131, ASP134 and ILE135.

CHIP_Helicon318 (PDB code: 8EI0). CHIP by Helicon318: Asn34, Phe37, Val38, Tyr49, Asn65, Leu68, Leu71, Lys72, Ser93, Val94, Lys95, Phe98, Phe99, Gln102, Glu106, Leu129, Asn130, Phel31, Gly132, Asp134 and Ile135.

CUL5_Helicon314 (PDB code: 8EI2). CUL5 by Helicon314: Val35, Thr36, Lys37, Trp40, Phe41, Phe44, His48, Ile106, Lys109, Cys112 and Gln113.

VHL_Helicon313 (PDB code: 8EI3). VHL by Helicon313: Pro59, Arg60, Val62, Leu63, Arg64, Val66, Gly114, His115, Leu116, Thr133, Glu134, Leu135, Val137, Leu201, Thr202, Arg205 and Ile206.

WWP1_Helicon302 (PDB code: 8EI4). WWP1 by Helicon302: Glu702, Phe703, Ser706, Leu707, Trp709, Ile710, Glu717, Cys718, Gly719, Leu720, Glu721, Met722, Val726 and Met760.

WWP2_Helicon301 (PDB code: 8EI5). WWP2 by Helicon301: Glu650, Phe651, Asn653, Ser654, Ile655, Trp657, Ile658, Asn661, Asn662, Glu665, Cys666, Gly667, Leu668, Glu669, Leu670, Gln674, Tyr704 and Leu708.

WWP2_Helicon305 (PDB code: 8EI6). WWP2 by Helicon305: Tyr499, Phe495, Arg496, His500, Arg503, Phe504, His507, Ser508, Gly619, Lys620, Phe621, Leu747, Met748, Met752 and Glu789.

WWP2_Helicon304 (PDB code: 8EI7). WWP2 by Helicon304: Phe495, Arg496, Tyr499, His500, Gly557, Arg561, Phe565, His569, Gly619, Lys620, Phe621, Ile622, Asp623, Leu747, Met748, Cys750, Gly751, Met752, Gln753, Glu754, Glu789, Arg803, Leu804, Pro805 and Val806. WWP2_Helicon308 (PDB code: 8EI8). WWP2 by Helicon308: Phe495, Arg496, Tyr499, His500, Arg561, Glu562, Phe565, Leu566, Gly619, Lys620, Phe621, Ile622, Asp623, Leu747, Cys750, Gly751, Met752, Gln753, Glu754, Asp787, Glu789 and Arg803.

TABLE E11-6
Certain structural data collection and refinement information

| | PDB code | | | | | |
|---|---|---|---|---|---|---|
| | 8EI9 | | 8EIA | | 8EIB | |
| | Structure Name | | | | | |
| | β-catenin_MDM2_Helicon332 | | β-catenin_MDM2_Helicon333 | | β-catenin_MDM2_Helicon329 | |
| Full Name | Crystal structure of beta-catenin and | | Crystal structure of beta-catenin and | | Crystal structure of beta-catenin and | |
| | the MDM2 p53-binding domain in | | the MDM2 p53-binding domain in | | the MDM2 p53-binding domain in | |
| | complex with H332, a Helicon | | complex with H333, a Helicon | | complex with H329, a Helicon | |
| | Polypeptide | | Polypeptide | | Polypeptide | |
| Wavelength (Å) | 0.920 | | 0.920 | | 0.920 | |
| Resolution range (Å) | 48.01-3.90 | (4.36-3.90) | 48.03-3.60 | (3.94-3.60) | 20.17-3.76 | (4.20-3.76) |
| Space group | P 21 21 21 | | P 1 21 1 | | P 21 21 21 | |
| Unit cell | | | | | | |
| a, b, c (Å) | 87.04 95.32 166.72 | | 49.81 96.07 76.79 | | 55.62 69.83 165.47 | |
| α, β, γ (°) | 90 90 90 | | 90 106.62 90 | | 90 90 90 | |
| Total reflections | 76659 | (19433) | 41220 | (9723) | 84610 | (22310) |
| Unique reflections | 13180 | (3642) | 7517 | (1803) | 6955 | (1935) |
| Multiplicity | 5.8 | (5.3) | 5.5 | (5.4) | 12.2 | (11.5) |
| Completeness (%) | 99.8 | (99.4) | 92.8 | (94.2) | 99.3 | (99.9) |
| Mean I/sigma (I) | 3.2 | (1.1) | 5.3 | (1.7) | 5.1 | (2.7) |
| Wilson B-factor (Å²) | 63.57 | | 56.79 | | 56.12 | |
| R-merge | 0.545 | (1.54) | 0.241 | (0.912) | 0.698 | (1.27) |
| R-meas | 0.661 | (1.89) | 0.295 | (1.11) | 0.758 | (1.39) |
| R-pim | 0.369 | (1.09) | 0.167 | (0.625) | 0.294 | (0.560) |
| CC1/2 | 0.923 | (0.428) | 0.978 | (0.491) | 0.972 | (0.794) |
| Reflections used in refinement | 13130 | (1261) | 7500 | (759) | 6915 | (666) |
| Reflections used for R-free | 582 | (70) | 750 | (75) | 691 | (67) |
| R-work | 0.266 | (0.324) | 0.319 | (0.349) | 0.245 | (0.262) |
| R-free | 0.313 | (0.404) | 0.351 | (0.329) | 0.260 | (0.258) |
| Number of non-hydrogen atoms | 4817 | | 4553 | | 4757 | |
| macromolecules | 4803 | | 4539 | | 4743 | |
| ligands | 14 | | 14 | | 14 | |
| solvent | | | | | | |
| Protein residues | 621 | | 586 | | 614 | |
| RMS (bonds) (Å) | 0.005 | | 0.006 | | 0.009 | |
| RMS (angles) (°) | 0.72 | | 1.04 | | 1.37 | |
| Ramachandran favored (%) | 89.27 | | 85.81 | | 82.31 | |
| Ramachandran allowed (%) | 9.27 | | 10.73 | | 12.07 | |
| Ramachandran outliers (%) | 1.46 | | 3.46 | | 5.62 | |
| Rotamer outliers (%) | 0.95 | | 2.03 | | 2.51 | |
| Clashscore | 7.36 | | 22.81 | | 19.55 | |
| Average B-factor (Å²) | 84.49 | | 84.34 | | 80.46 | |
| macromolecules | 84.43 | | 84.24 | | 80.37 | |
| ligands | 105.47 | | 114.94 | | 111.61 | |
| solvent | | | | | | |
| Crystallization conditions | 0.1M MES Sodium Salt pH 6.5, | | 0.1M Potassium chloride, 0.1M | | 0.2M Calcium acetate pH 7.5, 20% | |
| | 10% v/v 2-Propanol, 25% v/v | | HEPES pH 7, 15% w/v PEG 5000 | | w/v PEG 3350 | |
| | PEG 400 | | MME | | | |
| | PDB code | | | | | |
| | 8EIC | | 8EHZ | | 8EI0 | |
| | Structure Name | | | | | |
| | β-catenin_MDM2_Helicon330 | | CHIP_Helicon317 | | CHIP_Helicon318 | |
| Full Name | Crystal structure of beta-catenin and | | Crystal structure of the STUB1 TPR | | Crystal structure of the STUB1 TPR | |
| | the MDM2 p53-binding domain in | | domain in complex with H317, a | | domain in complex with H318, a | |
| | complex with H330, a Helicon | | Helicon Polypeptide | | Helicon Polypeptide | |
| | Polypeptide | | | | | |
| Wavelength (Å) | 0.979 | | 1.000 | | 1.033 | |
| Resolution range (Å) | 46.36-2.62 | (2.74-2.62) | 43.08-2.06 | (2.12-2.06) | 43.11-1.47 | (1.50-1.47) |
| Space group | P 21 21 21 | | P 32 | | C 2 | |
| Unit cell | | | | | | |
| a, b, c (Å) | 56.11 71.54 164.56 | | 63.21 63.21 69.82 | | 82.56 46.96 53.62 | |
| α, β, γ (°) | 90 90 90 | | 90 90 120 | | 90 126.48 90 | |
| Total reflections | 267056 | (31979) | 202213 | (15637) | 139635 | (6682) |
| Unique reflections | 20670 | (2482) | 19293 | (1471) | 27179 | (1284) |
| Multiplicity | 12.9 | (12.9) | 10.5 | (10.6) | 5.1 | (5.2) |
| Completeness (%) | 99.9 | (99.8) | 100.0 | (100.0) | 96.7 | (94.5) |
| Mean I/sigma (I) | 14.4 | (2.4) | 15.2 | (2.6) | 18.3 | (2.2) |
| Wilson B-factor (Å²) | 47.13 | | 41.14 | | 19.02 | |
| R-merge | 0.139 | (1.30) | 0.078 | (0.796) | 0.036 | (0.650) |
| R-meas | 0.150 | (1.41) | 0.087 | (0.884) | 0.045 | (0.815) |
| R-pim | 0.057 | (0.538) | 0.038 | (0.382) | 0.027 | (0.487) |
| CC1/2 | 0.999 | (0.685) | 0.998 | (0.938) | 0.999 | (0.758) |
| Reflections used in refinement | 20615 | (2023) | 19236 | (1897) | 26777 | (2611) |
| Reflections used for R-free | 992 | (107) | 1935 | (185) | 1362 | (121) |
| R-work | 0.224 | (0.303) | 0.241 | (0.344) | 0.194 | (0.322) |
| R-free | 0.262 | (0.346) | 0.267 | (0.342) | 0.210 | (0.338) |
| Number of non-hydrogen atoms | 4800 | | 2279 | | 1350 | |
| macromolecules | 4775 | | 2227 | | 1189 | |
| ligands | 14 | | 34 | | 31 | |
| solvent | 11 | | 18 | | 130 | |
| Protein residues | 619 | | 278 | | 144 | |
| RMS (bonds) (Å) | 0.01 | | 0.006 | | 0.008 | |
| RMS (angles) (°) | 1.05 | | 0.79 | | 1.07 | |
| Ramachandran favored (%) | 95.59 | | 94.03 | | 97.84 | |
| Ramachandran allowed (%) | 3.92 | | 5.97 | | 2.16 | |
| Ramachandran outliers (%) | 0.49 | | 0 | | 0 | |
| Rotamer outliers (%) | 1.73 | | 0 | | 1.64 | |
| Clashscore | 10.7 | | 3.81 | | 5.02 | |
| Average B-factor (Å²) | 71.4 | | 60.79 | | 26.37 | |
| macromolecules | 71.4 | | 60.71 | | 25.27 | |
| ligands | 86.83 | | 71.8 | | 37.04 | |
| solvent | 52.39 | | 49.92 | | 33.9 | |
| Crystallization conditions | 4M Na Form | | 0.1M Sodium acetate, 0.1M MES | | 0.2M Ammonium sulfate, | |
| | | | pH 6.0, 15% w/v PEG 4000 | | 30% w/v PEG 8000. | |
| | PDB code | | | | | |
| | 8EI1 | | 8EI2 | | 8EI3 | |
| | Structure Name | | | | | |
| | CUL4B_Helicon316 | | CUL5_Helicon314 | | VHL_Helicon313 | |
| Full Name | Crystal structure of the N-terminal | | Crystal structure of the N-terminal | | Crystal structure of VHL in | |
| | domain of CUL4B in complex with | | domain of CUL5 in complex with | | complex with H313, a Helicon | |
| | H316, a Helicon Polypeptide | | H314, a Helicon Polypeptide | | Polypeptide | |
| Wavelength (Å) | 0.976 | | 1.000 | | 1.000 | |
| Resolution range (Å) | 52.36-2.89 | (3.00-2.89) | 47.88-2.80 | (2.95-2.80) | 47.08-3.49 | (3.82-3.49) |
| Space group | P 43 21 2 | | P 6 | | P 41 | |
| Unit cell | | | | | | |
| a, b, c (Å) | 99.65 99.65 366.49 | | 173.03 173.03 57.48 | | 47.08 47.08 359.58 | |
| α, β, γ (°) | 90 90 90 | | 90 90 120 | | 90 90 90 | |
| Total reflections | 1134593 | (121904) | 318680 | (48322) | 89660 | (21010) |
| Unique reflections | 42685 | (4410) | 24154 | (3518) | 9977 | (2380) |
| Multiplicity | 26.6 | (27.6) | 13.2 | (13.7) | 9.0 | (8.8) |
| Completeness (%) | 100.0 | (100.0) | 98.2 | (99.3) | 100.0 | (100.0) |
| Mean I/sigma (I) | 23.9 | (2.4) | 5.0 | (1.8) | 6.5 | (2.6) |
| Wilson B-factor (Å²) | 72.97 | | 47.98 | | 53.11 | |
| R-merge | 0.110 | (1.84) | 0.322 | (1.12) | 0.287 | (0.726) |
| R-meas | 0.114 | (1.90) | 0.346 | (1.20) | 0.326 | (0.829) |
| R-pim | 0.030 | (0.498) | 0.125 | (0.430) | 0.152 | (0.389) |
| CC1/2 | 1.000 | (0.882) | 0.988 | (0.888) | 0.987 | (0.897) |
| Reflections used in refinement | 42538 | (4147) | 16542 | (442) | 9896 | (1033) |
| Reflections used for R-free | 2101 | (225) | 845 | (31) | 476 | (53) |
| R-work | 0.212 | (0.360) | 0.339 | (0.407) | 0.216 | (0.305) |
| R-free | 0.255 | (0.419) | 0.358 | (0.574) | 0.282 | (0.405) |
| Number of non-hydrogen atoms | 11809 | | 3067 | | 5748 | |
| macromolecules | 11745 | | 3053 | | 5734 | |
| ligands | 64 | | 14 | | 14 | |
| solvent | | | | | | |
| Protein residues | 1427 | | 373 | | 709 | |
| RMS (bonds) (Å) | 0.005 | | 0.009 | | 0.003 | |
| RMS (angles) (°) | 0.85 | | 1 | | 0.58 | |
| Ramachandran favored (%) | 96.24 | | 90.19 | | 91.9 | |
| Ramachandran allowed (%) | 2.91 | | 8.45 | | 6.22 | |
| Ramachandran outliers (%) | 0.85 | | 1.36 | | 1.88 | |
| Rotamer outliers (%) | 2.8 | | 0.6 | | 0.62 | |
| Clashscore | 7.41 | | 13.14 | | 7.15 | |
| Average B-factor (Å²) | 90.35 | | 20.32 | | 68.24 | |
| macromolecules | 90.15 | | 20.22 | | 68.19 | |
| ligands | 126.15 | | 41.2 | | 87.57 | |
| solvent | | | | | | |
| Crystallization conditions | 0.2M Lithium sulfate, 0.1M Tris | | 30% w/v PEG 8000, 0.1M MES | | 0.1M Tris pH 7.0, 20% w/v | |
| | pH 8.5, 30% w/v PEG 4000 | | Sodium Salt pH 6.5, 0.2M | | PEG 1000 | |
| | | | Ammonium Sulfate, 4% v/v | | | |
| | | | 1,3-Propanediol | | | |
| | PDB code | | | | | |
| | 8EI4 | | 8EI5 | | 8EI6 | |
| | Structure Name | | | | | |
| | WWP1_Helicon302 | | WWP2_Helicon301 | | WWP2_Helicon305 | |
| Full Name | Crystal structure of the WWP1 | | Crystal structure of the WWP2 | | Crystal structure of the WWP2 | |
| | HECT domain in complex with | | HECT domain in complex with | | HECT domain in complex with | |
| | H302, a Helicon Polypeptide | | H301, a Helicon Polypeptide | | H305, a Helicon Polypeptide | |
| Wavelength (Å) | 1.181 | | 1.033 | | 1.000 | |
| Resolution range (Å) | 45.18-2.43 | (2.52-2.43) | 45.40-2.60 | (2.68-2.60) | 49.07-3.60 | (3.94-3.60) |
| Space group | P1 | | P 21 | | P 21 21 21 | |
| Unit cell | | | | | | |
| a, b, c (Å) | 45.42 51.21 58.64 | | 120.66 63.10 122.26 | | 67.62 71.32 211.57 | |
| α, β, γ (°) | 112.09 99.61 102.51 | | 90 96.78 90 | | 90 90 90 | |
| Total reflections | 43024 | (3472) | 289797 | (23732) | 163370 | (39244) |
| Unique reflections | 16258 | (1472) | 56697 | (4596) | 12498 | (2921) |
| Multiplicity | 2.6 | (2.4) | 5.1 | (5.2) | 13.1 | (13.4) |
| Completeness (%) | 94.0 | (80.3) | 99.9 | (99.9) | 99.9 | (99.9) |
| Mean I/sigma (I) | 11.3 | (2.1) | 7.6 | (2.2) | 10.2 | (2.7) |
| Wilson B-factor (Å²) | 35.41 | | 29.51 | | 99.13 | |
| R-merge | 0.054 | (0.310) | 0.139 | (0.616) | 0.190 | (1.056) |
| R-meas | 0.077 | (0.438) | 0.175 | (0.773) | 0.205 | (1.139) |
| R-pim | 0.054 | (0.310) | 0.103 | (0.460) | 0.077 | (0.425) |
| CC1/2 | 0.997 | (0.872) | 0.995 | (0.827) | 0.998 | (0.979) |
| Reflections used in refinement | 15431 | (1326) | 56014 | (5551) | 9414 | (101) |
| Reflections used for R-free | 785 | (64) | 2658 | (246) | 957 | (12) |
| R-work | 0.220 | (0.328) | 0.214 | (0.270) | 0.259 | (0.328) |
| R-free | 0.280 | (0.477) | 0.261 | (0.306) | 0.279 | (0.275) |

| | | | |
|---|---|---|---|
| Number of non-hydrogen atoms | 3210 | 13513 | 6492 |
| macromolecules | 3145 | 13036 | 6464 |
| ligands | 30 | 158 | 28 |
| solvent | 35 | 319 | |
| Protein residues | 381 | 1542 | 766 |
| RMS (bonds) (Å) | 0.009 | 0.006 | 0.004 |
| RMS (angles) (°) | 1.06 | 0.97 | 0.85 |
| Ramachandran favored (%) | 95.44 | 97.38 | 91.78 |
| Ramachandran allowed (%) | 2.95 | 2.36 | 7.96 |
| Ramachandran outliers (%) | 1.61 | 0.26 | 0.27 |
| Rotamer outliers (%) | 0.59 | 4.45 | 0.43 |
| Clashscore | 8.29 | 5.93 | 9.37 |
| Average B-factor (Å²) | 45.6 | 39.63 | 78.69 |
| macromolecules | 45.51 | 39.61 | 78.63 |
| ligands | 56.1 | 51.4 | 92.73 |
| solvent | 44.83 | 34.75 | |
| Crystallization conditions | 0.2M Sodium acetate, 0.1M | 0.1M Sodium acetate, 0.1M sodium | 18% v/v 2-Propanol, 0.1M Sodium |
| | Sodium citrate pH 5.5, 5% w/v | citrate pH 6.0, 10% PEG 4000 | citrate pH 5.5, 20% w/v PEG 4,000 |
| | PEG 4000 | | |

| | PDB code | | | |
|---|---|---|---|---|
| | 8EI7 | | 8EI8 | |
| | Structure Name | | | |
| | WWP2_Helicon304 | | WWP2_Helicon308 | |
| Full Name | Crystal structure of the WWP2 | | Crystal structure of the WWP2 | |
| | HECT domain in complex with | | HECT domain in complex with | |
| | H304, a Helicon Polypeptide | | H308, a Helicon Polypeptide | |
| Wavelength (Å) | 1.033 | | 1.000 | |
| Resolution range (Å) | 44.69-2.22 | (2.29-2.22) | 43.77-2.90 | (3.08-2.90) |
| Space group | P 21 | | C 2 2 21 | |
| Unit cell | | | | |
| a, b, c (Å) | 62.16 64.30 103.69 | | 111.38 117.81 65.39 | |
| α, β, γ (°) | 90 90 90 | | 90 90 90 | |
| Total reflections | 206480 | (18955) | 129994 | (21614) |
| Unique reflections | 40576 | (3697) | 9862 | (1564) |
| Multiplicity | 5.1 | (5.1) | 13.2 | (13.8) |
| Completeness (%) | 99.8 | (99.8) | 100.0 | (100.0) |
| Mean I/sigma (I) | 13.1 | (2.3) | 8.0 | (2.4) |
| Wilson B-factor (Å²) | 35.97 | | 46.92 | |
| R-merge | 0.075 | (0.709) | 0.248 | (1.08) |
| R-meas | 0.093 | (0.882) | 0.268 | (1.16) |
| R-pim | 0.054 | (0.517) | 0.101 | (0.430) |
| CC1/2 | 0.998 | (0.720) | 0.992 | (0.814) |
| Reflections used in refinement | 40032 | (3950) | 9858 | (968) |
| Reflections used for R-free | 2013 | (180) | 486 | (63) |
| R-work | 0.200 | (0.271) | 0.236 | (0.312) |
| R-free | 0.263 | (0.352) | 0.303 | (0.457) |

| | | |
|---|---|---|
| Number of non-hydrogen atoms | 6764 | 3355 |
| macromolecules | 6481 | 3306 |
| ligands | 89 | 49 |
| solvent | 194 | |
| Protein residues | 763 | 391 |
| RMS (bonds) (Å) | 0.011 | 0.006 |
| RMS (angles) (°) | 1.27 | 0.9 |
| Ramachandran favored (%) | 97.35 | 92.76 |
| Ramachandran allowed (%) | 2.12 | 6.46 |
| Ramachandran outliers (%) | 0.53 | 0.78 |
| Rotamer outliers (%) | 2.84 | 1.12 |
| Clashscore | 8.46 | 10.57 |
| Average B-factor (Å²) | 47.92 | 61.15 |
| macromolecules | 48.04 | 61.05 |
| ligands | 49.64 | 68.02 |
| solvent | 43.11 | |
| Crystallization conditions | 0.2M magnesium chloride, 0.1M | 0.2M Magnesium Chloride, 0.1M |
| | Bis-tris pH 7.5, 30% PEG 3350 | Bis-Tris pH 6.5, 20% PEG 3350 |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described in the present disclosure. It is, therefore, to be understood that the foregoing embodiments are presented by way of example only and that, provided technologies, including those to be claimed, may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1-2145. (canceled)

2146. An agent, comprising:
X⁸⁶X⁻¹X⁰X¹X²X³X⁴X⁵X⁶X⁷X⁸X⁹X¹⁰X¹¹X¹²X¹³X¹⁴X⁵X¹⁶X¹⁷,
wherein:
each of X⁸⁶, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁵ comprises a side chain comprising a polar group;
X⁶ comprises a hydrophobic side chain;
X⁹ comprises a side chain comprising a polar group;
X¹⁰ comprises a hydrophobic side chain;
X¹² comprises a hydrophobic side chain;
X¹³ comprises a hydrophobic side chain; and
X¹⁴ comprises a side chain comprising an acidic group; or
an agent, comprising:
X⁸⁷X⁻¹X⁰X¹X²X³X⁴X⁵X⁶X⁷X⁸X⁹X¹⁰X¹¹X¹²X¹³X¹⁴X⁵X¹⁶X¹⁷,
wherein:
each of X⁸⁷, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁰ comprises a hydrophobic side chain or a side chain comprising an aromatic group;
X¹ comprises a hydrophobic side chain or a side chain comprising an aromatic group; and
X⁹ comprises a hydrophobic side chain; or
an agent, comprising:
X⁸⁸X⁻¹X⁰X¹X²X³X⁴X⁵X⁶X⁷X⁸X⁹X¹⁰X¹¹X¹²X¹³X¹⁴X⁵X¹⁶X¹⁷,
wherein:
each of X⁸⁸, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X² comprises a hydrophobic side chain or a side chain comprising an aromatic group;
X³ comprises a side chain comprising a polar group;
X⁵ comprises a hydrophobic side chain;
X⁶ comprises a side chain comprising an acidic group;
X⁹ comprises a hydrophobic side chain or a side chain comprising an aromatic group;
X¹⁰ comprises a hydrophobic side chain;
X¹² comprises a side chain comprising an acidic group;
X¹³ comprises a side chain comprising a polar group;
X¹⁴ comprises a hydrophobic side chain or a side chain comprising an aromatic group; and
X¹⁵ comprises a hydrophobic side chain; or
an agent, comprising:
X⁸⁹X⁻¹X⁰X¹X²X³X⁴X⁵X⁶X⁷X⁸X⁹X¹⁰X¹¹X¹²X¹³X¹⁴X¹⁵X¹⁶X¹⁷,
wherein:
each of X⁸⁹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁹ comprises a side chain comprising a polar group;
X¹⁰ comprises a hydrophobic side chain;
X¹² comprises a hydrophobic side chain;
X¹³ comprises a hydrophobic side chain; and
X¹⁴ comprises a side chain comprising an acidic group; or
an agent, comprising:
X⁹⁰X⁻¹X⁰X¹X²X³X⁴X⁵X⁶X⁷X⁸X⁹X¹⁰X¹¹X¹²X¹³X¹⁴X¹⁵X¹⁶X¹⁷,
wherein:
each of X⁹⁰, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X¹ comprises a hydrophobic side chain;
X² comprises a side chain comprising an acidic group;
X³ comprises a hydrophobic side chain;
X⁶ comprises a side chain comprising an aromatic group;
X⁹ comprises a side chain comprising an acidic group;
X¹⁰ comprises a hydrophobic side chain;
X¹² comprises a hydrophobic side chain or a side chain comprising an aromatic group;
X¹³ comprises a hydrophobic side chain; and
X¹⁴ comprises a hydrophobic side chain or a side chain comprising an aromatic group; or
an agent, comprising:
X⁹⁴X⁻¹X⁰X¹X²X³X⁴X⁵X⁶X⁷X⁸X⁹X¹⁰X¹¹X¹²X¹³X¹⁴X¹⁵X¹⁶X¹⁷,
wherein:
each of X⁹⁴, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁹ comprises a side chain comprising an acid group;
X¹⁰ comprises a hydrophobic side chain;
X¹² comprises a hydrophobic side chain or a side chain comprising an aromatic group;
X¹³ comprises a hydrophobic side chain;
X¹⁴ comprises a hydrophobic side chain or a side chain comprising an aromatic group;
X¹⁵ comprises a hydrophobic side chain or a side chain comprising an aromatic group;
X¹⁶ comprises a hydrophobic side chain; and
X¹⁷ comprises a hydrophobic side chain or a side chain comprising an aromatic group; or
an agent, comprising:
X⁹¹X⁻¹X⁰X¹X²X³X⁴X⁵X⁶X⁷X⁸X⁹X¹⁰X¹¹X¹²X¹³X¹⁴X¹⁵X¹⁶X¹⁷,
wherein:
each of X⁹¹, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X³ comprises a hydrophobic side chain;
X⁵ comprises a hydrophobic side chain or a side chain comprising an aromatic group;
X⁶ comprises a side chain comprising a polar group;
X⁹ comprises a hydrophobic side chain or a side chain comprising an aromatic group;
X¹² comprises a hydrophobic side chain;
X¹⁴ comprises a side chain comprising an acidic group;
X¹⁵ comprises a side chain comprising an acidic group; and
X¹⁶ comprises a hydrophobic side chain or a side chain comprising an aromatic group; or
an agent, comprising:
X⁹²X²X³X⁴X⁵X⁶X⁷X⁸X⁹X¹⁰X¹¹X¹²X¹³X¹⁴X¹⁵X¹⁶X¹⁷X¹⁸X¹⁹X²⁰,
wherein:
each of X⁹², X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X³ comprises a side chain comprising an acidic group;
X⁵ comprises a hydrophobic side chain or a side chain comprising an aromatic group;
X⁹ comprises a hydrophobic side chain or a side chain comprising an aromatic group;
X¹² comprises a hydrophobic side chain; and
X¹⁵ comprises a hydrophobic side chain; or
an agent, comprising:
X⁹³X⁻¹X⁰X¹X²X³X⁴X⁵X⁶X⁷X⁸X⁹X¹⁰X¹¹X¹²X¹³X¹⁴X¹⁵X¹⁶X¹⁷,
wherein:
each of X⁹³, X⁻¹, X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue;
X⁵ comprises a hydrophobic side chain or a side chain comprising an aromatic group;
X⁹ comprises a hydrophobic side chain or a side chain comprising an aromatic group;
X¹¹ comprises a hydrophobic side chain;
X¹⁶ comprises a hydrophobic side chain or a side chain comprising an aromatic group; and
X¹⁷ comprises an acidic side chain.

2147. An agent, comprising:
X³¹X²X³X⁴X⁵X⁶X⁷X⁸X⁹X¹⁰X¹¹X¹²X¹³X¹⁴,
wherein:
each of X³¹, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising an acidic group;
X⁵ comprises a hydrophobic side chain;
X⁶ comprises a hydrophobic side chain;
X⁹ comprises a side chain comprising an aromatic group or a basic group; and
X¹⁰ comprises a hydrophobic side chain; or
an agent, comprising:
X³²X²X³X⁴X⁵X⁶X⁷X⁸X⁹X¹⁰X¹¹X¹²X¹³X¹⁴,
wherein:
each of X³², X², X³, X⁴, X⁵, X⁶, X⁷, X⁸, X⁹, X¹⁰, X¹¹, X¹², X¹³, and X¹⁴ is independently an amino acid residue;
X² comprises a hydrophobic side chain;
X³ comprises a side chain comprising an acidic group;
X⁵ comprises a side chain comprising an aromatic group;
X⁶ comprises a hydrophobic side chain; and
X¹⁰ comprises a side chain comprising an acidic group;
an agent, comprising:
X⁴X⁵X⁶X⁷X⁸X⁹X¹⁰X¹¹X¹²X¹³X¹⁴,
wherein:
each of X⁴, X⁵, X⁶, X⁷, X⁸, X⁹, X¹⁰, X¹¹, X¹², X¹³, and X¹⁴ is independently an amino acid residue;
X⁵ comprises a side chain comprising an acidic group;
X⁶ comprises a side chain comprising an aromatic group;
X¹⁰ comprises a side chain comprising an aromatic group;
X¹³comprises a hydrophobic side chain; and
X¹⁴ comprises a hydrophobic side chain; or
an agent, comprising:
X³⁴X²X³X⁴X⁵X⁶X⁷X⁸X⁹X¹⁰X¹¹X¹²X¹³X¹⁴,
wherein:
each of X³⁴, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group;
X⁵ comprises a side chain comprising an acidic side chain;
X⁶ comprises a side chain comprising a basic group or an aromatic group;
X⁹ comprises a hydrophobic side chain; and
X¹⁰ comprises a hydrophobic side chain; or
an agent, comprising:
X³⁵X²X³X⁴X⁵X⁶X⁷X⁸X⁹X¹⁰X¹¹X¹²X¹³X¹⁴,
wherein:
each of X³⁵, X², X³, X⁴, X⁵, X⁶, X⁷, X⁸, X⁹, X¹⁰, X¹¹, X¹², X¹³, and X¹⁴ is independently an amino acid residue;
X⁵ comprises a side chain comprising an aromatic group;
X⁶ comprises a side chain comprising an acidic group;
X⁹ comprises a side chain comprising an aromatic group; and
X¹³ comprises a side chain comprising an aromatic group; or
an agent, comprising:
X⁴¹X²X³X⁴X⁵X⁶X⁷X⁸X⁹X¹⁰X¹¹X¹²X¹³X¹⁴,
wherein:
each of X⁴¹, X², X³, X⁴, X⁵, X⁶, X⁷, X⁸, X⁹, X¹⁰, X¹¹, X¹², X¹³, and X¹⁴ is independently an amino acid residue;
X⁶ comprises a side chain comprising an aromatic group;
X⁹ comprises a hydrophobic side chain;
X¹⁰ comprises a side chain comprising an aromatic group; and
X¹² comprises a side chain comprising an acidic group; or
an agent, comprising:
X⁴²X²X³X⁴X⁵X⁶X⁷X⁸X⁹X¹⁰X¹¹X¹²X¹³X¹⁴,
wherein:
each of X⁴², X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group;
X⁵ comprises a hydrophobic side chain; and
X⁶ comprises a side chain comprising an aromatic group; or
an agent, comprising:
X⁴³X²X³X⁴X⁵X⁶X⁷X⁸X⁹X¹⁰X¹¹X¹²X¹³X¹⁴,
wherein:
each of X⁴³, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an acidic group;
X³ comprises a side chain comprising an aromatic group;
X⁵ comprises a hydrophobic side chain;
X⁶ comprises a side chain comprising a polar group or an aromatic group; and
X¹⁰ comprises a side chain comprising an acidic group; or
an agent, comprising:
X⁴⁴X²X³X⁴X⁵X⁶X⁷X⁸X⁹X¹⁰X¹¹X¹²X¹³X¹⁴,
wherein:
each of X⁴⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain;
X¹³ comprises a side chain comprising a polar group; and
X¹⁴ comprises a side chain comprising s polar group or an aromatic group; or
an agent, comprising:
an agent, comprising:
X⁴⁵X²X³X⁴X⁵X⁶X⁷X⁸X⁹X¹⁰X¹¹X¹²X¹³X¹⁴,
wherein:
each of X⁴⁵, X², X³, X⁴, X⁵, X⁶, X⁷, X⁸, X⁹, X¹⁰, X¹¹, X¹², X¹³, and X¹⁴ is independently an amino acid residue;
X⁶ comprises a side chain comprising an acidic group;
X⁹ comprises a hydrophobic side chain;
X¹² comprises a side chain comprising a polar group; and
X¹³ comprises a side chain comprising a polar group or an aromatic group; or
an agent, comprising:
X⁴⁶X²X³X⁴X⁵X⁶X⁷X⁸X⁹X¹⁰X¹¹X¹²X¹³X¹⁴,
wherein:
each of X³¹, X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain or a side chain comprising an aromatic group;
X⁶ comprises a side chain comprising a polar group;
X⁹ comprises a hydrophobic side chain
X¹⁰ comprises a side chain comprising an aromatic group; or
an agent, comprising:
X⁴⁷X²X³X⁴X⁵X⁶X⁷X⁸X⁹X¹⁰X¹¹X¹²X¹³X¹⁴,
wherein:
each of X³¹, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising a polar group;
X⁵ comprises a hydrophobic side chain;
X⁶ comprises a side chain comprising a polar group or an aromatic group; and
X⁹ comprises a hydrophobic side chain; or
an agent, comprising:
X⁴⁸X²X³X⁴X⁵X⁶X⁷X⁸X⁹X¹⁰X¹¹X¹²X¹³X¹⁴,
wherein:
each of X⁴⁸, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising a basic group or an aromatic group;
X¹⁰ comprises a side chain comprising an aromatic group;
X¹² comprises a hydrophobic side chain; and
X¹³ comprises a hydrophobic side chain; or
an agent, comprising:
X⁵¹X²X³X⁴X⁵X⁶X⁷X⁸X⁹X¹⁰X¹¹X¹²X¹³X¹⁴,
wherein:
each of X⁵¹, X², X³, X⁴, X⁵, X⁶, X⁷, X⁸, X⁹, X¹⁰, X¹¹, X¹², X¹³, and X¹⁴ is independently an amino acid residue;
X¹ comprises a side chain comprising an aromatic group;
X² comprises a side chain comprising an aromatic group;
X⁶ comprises a hydrophobic side chain;
X⁹ comprises a hydrophobic side chain; and
X¹⁰ comprises a side chain comprising an acidic group; or
an agent, comprising
X⁵²X²X³X⁴X⁵X⁶X⁷X⁸X⁹X¹⁰X¹¹X¹²X¹³,
wherein:
each of X⁵², X², X³, X⁴, X⁵, X⁶, X⁷, X⁸, X⁹, X¹⁰, X¹¹, X¹², and X¹³ is independently an amino acid residue;
X⁵ comprises a hydrophobic side chain;
X⁸ comprises a side chain comprising an aromatic group; and
X⁹ comprises a side chain comprising an aromatic group; or
an agent, comprising
X⁵³X²X³X⁴X⁵X⁶X⁷X⁸X⁹X¹⁰X¹¹X¹²X¹³X¹⁴,
wherein:
each of X⁵³, X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising an acidic group;
X⁵ comprises a side chain comprising a basic or aromatic group;
X⁶ comprises a hydrophobic side chain; and
X⁹ comprises a side chain comprising an aromatic group or a polar group; or
an agent, comprising:
X⁵⁴X²X³X⁴X⁵X⁶X⁷X⁸X⁹X¹⁰X¹¹X¹²,
wherein:
each of X⁵⁴, X², X³, X⁴, X⁵, X⁶, X⁷, X⁸, X⁹, X¹⁰, X¹¹, and X¹² is independently an amino acid residue;
X⁵ comprises a side chain comprising an aromatic group or a basic group;
X⁶ comprises a hydrophobic side chain;
X⁸ comprises a hydrophobic side chain; and
X⁹ comprises a side chain comprising an aromatic group; or
an agent, comprising:
X⁶¹X³X⁴X⁵X⁶X⁷X⁸X⁹X¹⁰X¹¹X¹²X¹³X¹⁴,
wherein:
each of X⁶¹, X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group;
X⁶ comprises a hydrophobic side chain or a side chain comprising an aromatic group; and
X⁹ comprises a side chain comprising a basic group; or
an agent, comprising:
X⁶²X²X³X⁴X⁵X⁶X⁷X⁸X⁹X¹⁰X¹¹X¹²X¹³X¹⁴,
wherein:
each of X⁶², X², X³, X⁴, X⁵, X⁶, X⁷, X⁸, X⁹, X¹⁰, X¹¹, X¹², X¹³, and X¹⁴ is independently an amino acid residue;
X³ comprises a side chain comprising a polar group or an acidic group;
X⁶ comprises a hydrophobic side chain or a side chain comprising an aromatic group; and
X⁹ comprises a side chain comprising an aromatic group; or

2148. A method for modulating a beta-catenin activity, reducing beta-catenin interaction with axin, reducing beta-catenin interaction with TCF, modulating a beta-catenin activity in a system, reducing beta-catenin interaction with axin in a system, or reducing beta-catenin interaction with TCF in a system, comprising contacting beta-catenin, or administering or delivering to the system, with an agent of claim 2147.

2149. A method for treating a condition, disorder or disease, comprising administering or delivering to a subject suffering therefrom an effective amount of an agent of claim 2147, optionally wherein the condition, disorder or disease is associated with beta-catenin, optionally wherein the condition, disorder or disease is cancer.

2150. A method for modulating a RNF31 activity or modulating a RNF31 activity in a system, comprising contacting RNF31, or administering or delivering to the system, with an agent claim 2147; or

2151. A method for treating a condition, disorder or disease, comprising administering or delivering to a subject suffering therefrom an effective amount of an agent of claim 2147, optionally wherein the condition, disorder or disease is associated with RNF3, optionally wherein the condition, disorder or disease is cancer.

2152. A method for modulating a CDK2 activity or modulating a CDK2 activity in a system, comprising contacting CDK2, or administering or delivering to the system, with an agent of claim 2147.

2153. A method for treating a condition, disorder or disease, comprising administering or delivering to a subject suffering therefrom an effective amount of an agent of claim 2147, optionally wherein the condition, disorder or disease is associated with CDK2, optionally wherein the condition, disorder or disease is cancer.

2154. A method for modulating a PPIA activity, inhibiting PPIA peptidyl-prolyl cis-trans isomerase activity, modulating a PPIA activity in a system, or inhibiting PPIA peptidyl-prolyl cis-trans isomerase activity in a system, comprising contacting PPIA with, or administering or delivering to the system, an agent of claim 2147.

2155. A method for treating a condition, disorder or disease, comprising administering or delivering to a subject suffering therefrom an effective amount of an agent of claim 2147, optionally wherein the condition, disorder or disease is associated with PPIA, optionally wherein the condition, disorder or disease is cancer.

2156. An agent, comprising:
X⁷¹X²X³X⁴X⁵X⁶X⁷X⁸X⁹X¹⁰X¹¹X¹²X¹³X¹⁴,
wherein:
each of X⁷¹, X², X³, X⁴, X⁵, X⁶, X⁷, X⁸, X⁹, X¹⁰, Xi, X¹², X, and X¹⁴ is independently an amino acid residue;
X¹⁰ comprises a hydrophobic side chain; or
an agent, comprising:
X⁷²X²X³X⁴X⁵X⁶X⁷X⁸X⁹X¹⁰X¹¹X¹²X¹³X¹⁴,
wherein:
each of X⁷², X², X³, X⁴, X⁵, X⁶, X⁷, X⁸, X⁹, X¹⁰, X¹¹, X¹², X¹³, and X¹⁴ is independently an amino acid residue;
X⁹ comprises a side chain comprising a polar group;
X¹³ comprises a hydrophobic side chain or a side chain comprising an aromatic group; and
X¹⁴ comprises a hydrophobic side chain or a side chain comprising an aromatic group; or
an agent, comprising:
X⁷³X²X³X⁴X⁵X⁶X⁷X⁸X⁹X¹⁰X¹¹X¹²X¹³X¹⁴,
wherein:
each of X⁷³, X², X³, X⁴, X⁵, X⁶, X⁷, X⁸, X⁹, X¹⁰, X¹¹, X¹², X¹³, and X¹⁴ is independently an amino acid residue; and
X² comprises a hydrophobic side chain or a side chain comprising an aromatic group; or
an agent, comprising:
X⁷⁴X²X³X⁴X⁵X⁶X⁷X⁸X⁹X¹⁰X¹¹X¹²X¹³X¹⁴,
wherein:
each of X⁷⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain; and
X¹² comprises a side chain comprising a basic group or an aromatic group; or
an agent, comprising:
X⁷⁵X²X³X⁴X⁵X⁶X⁷X⁸X⁹X¹⁰X¹¹X¹²X¹³X¹⁴,
wherein:
each of X⁷⁵, X², X³, X⁴, X⁵, X⁶, X⁷, X⁸, X⁹, X¹⁰, X¹¹, X¹², X¹³, and X¹⁴ is independently an amino acid residue;
X⁷⁵ comprises a hydrophobic side chain or a side chain comprising an aromatic group; and
X¹² comprises a side chain comprising an acidic group; or
an agent, comprising:
X⁷⁶X²X³X⁴X⁵X⁶X⁷X⁸X⁹X¹⁰X¹¹X¹²X¹³X¹⁴,
wherein:
each of X⁷⁶, X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain; and
X⁹ comprises a hydrophobic side chain or a side chain comprising an aromatic group; or
an agent, comprising:
X⁷⁷X²X³X⁴X⁵X⁶X⁷X⁸X⁹X¹⁰X¹¹X¹²X¹³X¹⁴,
wherein:
each of X⁷⁷, X², X³, X⁴, X⁵, X⁶, X⁷, X⁸, X⁹, X¹⁰, X¹¹, X¹², X¹³, and X¹⁴ is independently an amino acid residue;
X⁶ comprises a hydrophobic side chain; and
X⁹ comprises a hydrophobic side chain or a side chain comprising an aromatic group; or
an agent, comprising:
X⁷⁸X²X³X⁴X⁵X⁶X⁷X⁸X⁹X¹⁰X¹¹X¹²X¹³X¹⁴,
wherein:
each of X⁷⁸, X², X³, X⁴, X⁵, X⁶, X⁷, X⁸, X⁹, X¹⁰, X¹¹, X¹², X¹³, and X¹⁴ is independently an amino acid residue;
X⁵ comprises a side chain comprising an acidic group; and
X¹³ comprises an aliphatic side chain; or
an agent, comprising:
X⁷⁹X²X³X⁴X⁵X⁶X⁷X⁸X⁹X¹⁰X¹¹X¹²X¹³X¹⁴,
wherein:
each of X⁷⁹, X², X³, X⁴, X⁵, X⁶, X⁷, X⁸, X⁹, X¹⁰, X¹¹, X¹², X¹³, and X¹⁴ is independently an amino acid residue;
X¹³ comprises a hydrophobic side chain or a side chain comprising an aromatic group; and
X¹⁴ comprises a hydrophobic side chain or a side chain comprising an aromatic group; or
an agent, comprising:
[X⁸⁰]p1[X²]p2[X³]p3X⁴X⁵X⁶X⁷X⁸X⁹X¹⁰X¹¹X¹²X¹³X¹⁴,
wherein:
each of p1, p2 and p3 is independently 0 or 1;
each of X⁸⁰, X², X³, X⁴, X⁵, X⁶, X⁷, X⁸, X⁹, X¹⁰, X¹¹, X¹², X¹³, and X¹⁴ is independently an amino acid residue;
X⁷ comprises a hydrophobic side chain; and
X⁹ comprises a hydrophobic side chain; or
an agent, comprising:
X⁸¹X²X³X⁴X⁵X⁶X⁷X⁸X⁹X¹⁰X¹¹X¹²X¹³X¹⁴,
wherein:
each of X⁸¹, X², X³, X⁴, X⁵, X⁶, X⁷, X⁸, X⁹, X¹⁰, X¹¹, X¹², X¹³, and X¹⁴ is independently an amino acid residue;
X⁵ comprises a hydrophobic side chain or a side chain comprising an aromatic group;
X⁶ comprises a side chain comprising a polar group;
X¹⁰ comprises a side chain comprising an acidic group; and
X¹⁴ comprises hydrophobic side chain or a side chain comprising an aromatic group; or
an agent, comprising:
X⁸²X²X³X⁴X⁵X⁶X⁷X⁸X⁹X¹⁰X¹¹X¹²X¹³X¹⁴,
wherein:
each of X⁸², X², X³, X⁴, X⁵, X⁶, X⁷, X⁸, X⁹, X¹⁰, X¹¹, X¹², X¹³, and X¹⁴ is independently an amino acid residue;
X⁹ comprises a hydrophobic side chain or a side chain comprising an aromatic group; and
X¹³ comprises a hydrophobic side chain or a side chain comprising an aromatic group; or
an agent, comprising:
X⁸³X²X³X⁴X⁵X⁶X⁷X⁸X⁹X¹⁰X¹¹X¹²X¹³X¹⁴,
wherein:
each of X⁸³, X², X³, X⁴, X⁵, X⁶, X⁷, X⁸, X⁹, X¹⁰, X¹¹, X¹², X¹³, and X¹⁴ is independently an amino acid residue;
X⁸³ comprises a hydrophobic side chain or a side chain comprising an aromatic group;
X⁵ comprises a side chain comprising a polar group; and
X⁹ comprises a hydrophobic side chain; or
an agent, comprising:
X⁸⁴X²X³X⁴X⁵X⁶X⁷X⁸X⁹X¹⁰X¹¹X¹²X¹³X¹⁴,
wherein:
each of X⁸⁴, X², X³, X⁴, X⁵, X⁶, X⁷, X⁸, X⁹, X¹⁰, X¹¹, X¹², X¹³, and X¹⁴ is independently an amino acid residue;
X¹⁰ comprises a hydrophobic side chain;
X¹³ comprises a hydrophobic side chain; and
X¹⁴ comprises a side chain comprising an acidic group; or
an agent, comprising:
X⁸⁵X²X³X⁴X⁵X⁶X⁷X⁸X⁹X¹⁰X¹¹X¹²X³X¹⁴,
wherein:
each of X⁸⁵, X², X³, X⁴, X⁵, X⁶, X⁷, X⁸, X⁹, X¹⁰, Xi, X¹², X, and X¹⁴ is independently an amino acid residue;
X³ comprises a side chain comprising an acidic or polar group;
X⁵ comprises a hydrophobic side chain or a side chain comprising an aromatic group;
X⁹ comprises a hydrophobic side chain or a side chain comprising an aromatic group; and
X¹² comprises a hydrophobic side chain.

2157. A method, comprising:
contacting a first collection of stapled peptides with a first target of interest so that a number of stapled peptides of the first collection binds to the target;
determining amino acid sequences of stapled peptides of the first collection that bind to the first target;
identifying one or more enriched amino acid residues shared by a cluster of stapled peptides of the first collection that bind to the first target independently at one or more enriched positions (“first collection enriched amino acid residues”);
contacting a second collection of stapled peptides with a second target of interest so that one or more stapled peptides of the second collection binds to the second target, wherein the second collection is enriched for one or more first collection enriched amino acid residues independently at one or more enriched positions or positions corresponding thereto; and
determining amino acid sequences of stapled peptides that bind to the second target.

2158. A method for recognizing or reprogramming a polypeptide surface, or for reprograming a polypeptide to cooperatively bind a target polypeptide, comprising contacting the polypeptide with an agent identified with the method of claim 2157.

2159. A method for modulating polypeptide interaction, comprising contacting two polypeptides with an agent of claim 2146.

2160. A method for modulating polypeptide interaction, comprising contacting two polypeptides with an agent of claim 2156.

2161. A method for modulating polypeptide interaction, comprising contacting two polypeptides with an agent identified with the method of claim 2157.

2162. A method for modulating level of a target polypeptide, or for degrading a target polypeptide, comprising contacting the target polypeptide with an agent of claim 2156.

2163. A method for modulating function or level of a target polypeptide, comprising contacting the target polypeptide with an agent of claim 2146.

2164. A method for modulating function or level of a target polypeptide, comprising contacting the target polypeptide with an agent of claim 2147.

2165. A method for modulating function or level of a target polypeptide, comprising contacting the target polypeptide with an agent identified with a method of claim 2157.


SEQUENCE LISTING (915 sequences)

SEQ ID NO: 1 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 2 - protein, 1072 aa, Homo sapiens
MPGEEEERAFLVAREELASALRRDSGQAFSLEQLRPLLASSLPLAARYLQLDAARLVRCNAHGEPRNYLNTLSTALNILEKYGRNLLSPQRPRYWRGVKFNNPVFRSTVDAVQGGRDVLRLYGYTEEQPDGLSFPEGQEEPDEHQVATVTLEVLLLRTELSLLLQNTHPRQQALEQLLEDKVEDDMLQLSEFDPLLREIAPGPLTTPSVPGSTPGPCFLCGSAPGTLHCPSCKQALCPACDHLFHGHPSRAHHLRQTLPGVLQGTHLSPSLPASAQPRPQSTSLLALGDSSLSSPNPASAHLPWHCAACAMLNEPWAVLCVACDRPRGCKGLGLGTEGPQGTGGLEPDLARGRWACQSCTFENEAAAVLCSICERPRLAQPPSLVVDSRDAGICLQPLQQGDALLASAQSQVWYCIHCTFCNSSPGWVCVMCNRTSSPIPAQHAPRPYASSLEKGPPKPGPPRRLSAPLPSSCGDPEKQRQDKMREEGLQLVSMIREGEAAGACPEEIFSALQYSGTEVPLQWLRSELPYVLEMVAELAGQQDPGLGAFSCQEARRAWLDRHGNLDEAVEECVRTRRRKVQELQSLGFGPEEGSLQALFQHGGDVSRALTELQRQRLEPFRQRLWDSGPEPTPSWDGPDKQSLVRRLLAVYALPSWGRAELALSLLQETPRNYELGDVVEAVRHSQDRAFLRRLLAQECAVCGWALPHNRMQALTSCECTICPDCFRQHFTIALKEKHITDMVCPACGRPDLTDDTQLLSYFSTLDIQLRESLEPDAYALFHKKLTEGVLMRDPKFLWCAQCSFGFIYEREQLEATCPQCHQTFCVRCKRQWEEQHRGRSCEDFQNWKRMNDPEYQAQGLAMYLQENGIDCPKCKFSYALARGGCMHFHCTQCRHQFCSGCYNAFYAKNKCPEPNCRVKKSLHGHHPRDCLFYLRDWTALRLQKLLQDNNVMFNTEPPAGARAVPGGGCRVIEQKEVPNGLRDEACGKETPAGYAGLCQAHYKEYLVSLINAHSLDPATLYEVEELETATERYLHVRPQPLAGEDPPAYQARLLQKLTEEVPLGQSIPRRRK

SEQ ID NO: 3 - protein, 298 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 4 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 5 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 6 - protein, 31 aa, Homo sapiens
PSKPDNPGEDAPAEDMARYYSALGHYINLIT

SEQ ID NO: 7 - protein, 31 aa, Ictalurus punctatus
PSKPENPGEDAAPEELAKYYTALRHYINLIT

SEQ ID NO: 8 - protein, 31 aa, Marmota marmota
PLEPVYPGDNATPEQMAQYAAELRRYINMLT

SEQ ID NO: 9 - protein, 31 aa, Myotis lucifugus
PSKPEKPGENASAEELARYYSALRHYINLIT

SEQ ID NO: 10 - protein, 31 aa, Betta splendens
PPKPENPGEDAPPEELAKYYTALRHYINLIT

SEQ ID NO: 11 - protein, 31 aa, Notechis scutatus
PPKPESPGENASPEEMAKYLADLRHYINLVT

SEQ ID NO: 12 - protein, 31 aa, Orcinus orca
PAKPEAPGSHASPEELKRYYLSLRHFLNLVT

SEQ ID NO: 13 - protein, 31 aa, Calypte anna
PPKPETPGDEASPEEVAKYFSALRHYINLVT

SEQ ID NO: 14 - protein, 31 aa, Podarcis muralis
PQQPEHPGEDASAEEMARYLSALRHYLNLVT

SEQ ID NO: 15 - protein, 31 aa, Vombatus ursinus
PSKPKPPSENASREELSRYYAALRQYLNLVT

SEQ ID NO: 16 - protein, 31 aa, Ornithorhynchus anatinus
PVKPQPPPDNATPEELAQYFASLRHYLNLVT

SEQ ID NO: 17 - protein, 31 aa, Otolemur garnettii
PLEPVYPGENATPEQMAQYAAELRRYINMLT

SEQ ID NO: 18 - protein, 31 aa, Gulo gulo
PSKPDNPGEDAPAEDMARYYSALRHYINLIT

SEQ ID NO: 19 - protein, 14 aa, synthetic construct
AGPAKPEAGEDASP

SEQ ID NO: 20 - protein, 4 aa, synthetic construct
DPAA

SEQ ID NO: 21 - DNA, 110 bp, synthetic construct
  source 1..110: mol_type = other DNA
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 55..57: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 67..69: note = This region may encompass a codon which encodes an amino acid that is not cysteine, lysine, proline, or glycine
  misc_difference 70..72: note = This region may encompass a codon which encodes an amino acid that is not cysteine, lysine, proline, or glycine
  misc_difference 76..78: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
catgcccgggtacctttctattctcactctgcggatccggcgnnnnnnnnntgcnnnnnngcagcannnnnntgtnnnnnnnnnggtggttctggctggggtcgtggttc

SEQ ID NO: 22 - DNA, 37 bp, synthetic construct
  source 1..37: mol_type = other DNA
catgtttcggccgaaccacgacctgcgccagaaccac

SEQ ID NO: 23 - protein, 20 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 8: note = Any amino acid
  VARIANT 12: note = F, I, L, or M
  VARIANT 13: note = Any amino acid
  VARIANT 15: note = Any amino acid
  VARIANT 16: note = F or Y
  VARIANT 17: note = F or Y
  VARIANT 18: note = Any amino acid
  VARIANT 19: note = Any amino acid
  VARIANT 20: note = Any amino acid
XXXXWECXEAAXXCXXXXXX

SEQ ID NO: 24 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnntgggaatgtnnngaagcagcawtknnntgtnnntwttwtnnnnnnnnnggtggttctggcgcaggtcgtggttc

SEQ ID NO: 25 - DNA, 52 bp, synthetic construct
  source 1..52: mol_type = other DNA
tcgtcggcagcgtcagatgtgtataagagacagttcgcaattcctttagtgg

SEQ ID NO: 26 - DNA, 58 bp, synthetic construct
  source 1..58: mol_type = other DNA
gtctcgtgggctcggagatgtgtataagagacagattttctgtatgggattttgctaa

SEQ ID NO: 27 - protein, 21 aa, synthetic construct
  SITE 1: note = Acetylated residue
  SITE 21: note = Amidated residue
  SITE 8..15: note = Cysteines are stapled
PMEQQAICFQAAWMCLADDWT

SEQ ID NO: 28 - protein, 21 aa, synthetic construct
  SITE 1: note = Acetylated residue
  SITE 21: note = Amidated residue
  SITE 8..15: note = Cysteines are stapled
PWKYEQVCYQAAWQCLSDDWD

SEQ ID NO: 29 - protein, 8 aa, synthetic construct
DYKDDDDK

SEQ ID NO: 30 - DNA, 127 bp, synthetic construct
  source 1..127: mol_type = other DNA
  misc_difference 57..59: note = This region may encompass a codon which encodes an amino acid that is not cysteine, lysine, proline, or glycine
  misc_difference 66..68: note = This region may encompass a codon which encodes an amino acid that is not cysteine, lysine, proline, or glycine
  misc_difference 69..71: note = This region may encompass a codon which encodes an amino acid that is not cysteine, lysine, proline, or glycine
  misc_difference 75..77: note = This region may encompass a codon which encodes an amino acid that is not cysteine, lysine, proline, or glycine
  misc_difference 78..80: note = This region may encompass a codon which encodes an amino acid that is not cysteine, lysine, proline, or glycine
  misc_difference 81..83: note = This region may encompass a codon which encodes an amino acid that is not cysteine, lysine, proline, or glycine
  misc_difference 87..89: note = This region may encompass a codon which encodes an amino acid that is not cysteine, lysine, proline, or glycine
  misc_difference 90..92: note = This region may encompass a codon which encodes an amino acid that is not cysteine, lysine, proline, or glycine
  misc_difference 99..101: note = This region may encompass a codon which encodes an amino acid that is not cysteine, lysine, proline, or glycine
gggccggtaccggcggcccgagccagccggcgtgtccgggcgatgatgcgagcattnnngatctgnnnnnntatnnnnnnnnnctgnnnnnntatctgnnngcggtggcgggtggtgagctcatttt

SEQ ID NO: 31 - DNA, 56 bp, synthetic construct
  source 1..56: mol_type = other DNA
aaaatggtaccggcggcccgagccagccggcgtgtccgggcgatgatgcgagcatt

SEQ ID NO: 32 - DNA, 27 bp, synthetic construct
  source 1..27: mol_type = other DNA
gtggaattgtgagcggataacaatttg

SEQ ID NO: 33 - DNA, 24 bp, synthetic construct
  source 1..24: mol_type = other DNA
gcgtaacgatctaaagttttgtcg

SEQ ID NO: 34 - DNA, 63 bp, synthetic construct
  source 1..63: mol_type = other DNA
tcgtcggcagcgtcagatgtgtataagagacagggcggattataaagatgacgatgacaaagg

SEQ ID NO: 35 - DNA, 62 bp, synthetic construct
  source 1..62: mol_type = other DNA
gtctcgtgggctcggagatgtgtataagagacagcgttagtaaatgaattttctgtatgagg

SEQ ID NO: 36 - protein, 32 aa, synthetic construct
  SITE 7: note = Cysteine stapled to cysteine in another sequence
GPSQPACPGDDASILDLQLYDLDLYDYLLAVA

SEQ ID NO: 37 - protein, 32 aa, synthetic construct
  SITE 7: note = Cysteine stapled to cysteine in another sequence
GPRRPRCPGDDASIFDLDMYFEWLDDYLTAVA

SEQ ID NO: 38 - DNA, 60 bp, synthetic construct
  source 1..60: mol_type = other DNA
tctcactctgcgccggaatgcattctggattgccatgtggcgcgcgtgtggggtggttct

SEQ ID NO: 39 - protein, 6 aa, synthetic construct
HHHHHH

SEQ ID NO: 40 - protein, 10 aa, synthetic construct
HHHHHHHHHH

SEQ ID NO: 41 - protein, 11 aa, synthetic construct
DSLEFIASKLA

SEQ ID NO: 42 - protein, 32 aa, synthetic construct
  SITE 1: note = Acetylated residue
  SITE 32: note = Amidated residue
LWWPDGSGSGGSPGQVPMRKRQLPASFWEEPR

SEQ ID NO: 43 - protein, 31 aa, synthetic construct
  SITE 1: note = Acetylated residue
  SITE 3: note = Cyclopentylalanine
  SITE 12: note = (S)-2-amino-3-(thiophen-3-yl)propanoic acid
  SITE 13: note = Benzothienylalanine
  SITE 19: note = Beta-alanine
  SITE 31: note = Amidated residue
  SITE 4..11: note = Cysteines are stapled
PDXCDDAAFNCXXQGSGSXLTFEHYWAQLTS

SEQ ID NO: 44 - protein, 11 aa, synthetic construct
  SITE 1: note = 5-Carboxyfluorescein beta-alanine
XSSGPTIEEVD

SEQ ID NO: 45 - protein, 13 aa, synthetic construct
  SITE 1: note = 5-Carboxyfluorescein beta-alanine
  SITE 13: note = Amidated residue
XLTFEHYWAQLTS

SEQ ID NO: 46 - protein, 17 aa, synthetic construct
  SITE 1: note = Acetylated residue
  SITE 17: note = Amidated residue
  SITE 7..14: note = Cysteines are stapled
DPATHRCEWAALHCELV

SEQ ID NO: 47 - protein, 17 aa, synthetic construct
  SITE 1: note = Acetylated residue
  SITE 17: note = Amidated residue
DPATHRCEWAALHCELV

SEQ ID NO: 48 - protein, 17 aa, synthetic construct
  SITE 1: note = Acetylated residue
  SITE 17: note = Amidated residue
  SITE 7..14: note = Cysteines are stapled
DPAAHRCEWAALHCELV

SEQ ID NO: 49 - protein, 17 aa, synthetic construct
  SITE 1: note = Acetylated residue
  SITE 17: note = Amidated residue
  SITE 7..14: note = Cysteines are stapled
DPATARCEWAALHCELV

SEQ ID NO: 50 - protein, 17 aa, synthetic construct
  SITE 1: note = Acetylated residue
  SITE 17: note = Amidated residue
  SITE 7..14: note = Cysteines are stapled
DPATHACEWAALHCELV

SEQ ID NO: 51 - protein, 17 aa, synthetic construct
  SITE 1: note = Acetylated residue
  SITE 17: note = Amidated residue
  SITE 7..14: note = Cysteines are stapled
DPATHRCAWAALHCELV

SEQ ID NO: 52 - protein, 17 aa, synthetic construct
  SITE 1: note = Acetylated residue
  SITE 17: note = Amidated residue
  SITE 7..14: note = Cysteines are stapled
DPATHRCEAAALHCELV

SEQ ID NO: 53 - protein, 17 aa, synthetic construct
  SITE 1: note = Acetylated residue
  SITE 17: note = Amidated residue
  SITE 7..14: note = Cysteines are stapled
DPATHRCEWAAAHCELV

SEQ ID NO: 54 - protein, 17 aa, synthetic construct
  SITE 1: note = Acetylated residue
  SITE 17: note = Amidated residue
  SITE 7..14: note = Cysteines are stapled
DPATHRCEWAALHCALV

SEQ ID NO: 55 - protein, 17 aa, synthetic construct
  SITE 1: note = Acetylated residue
  SITE 17: note = Amidated residue
  SITE 7..14: note = Cysteines are stapled
DPATHRCEWAALHCEAV

SEQ ID NO: 56 - protein, 17 aa, synthetic construct
  SITE 1: note = Acetylated residue
  SITE 17: note = Amidated residue
  SITE 7..14: note = Cysteines are stapled
DPATHRCEWAALHCELA

SEQ ID NO: 57 - protein, 17 aa, synthetic construct
  SITE 1: note = Acetylated residue
  SITE 17: note = Amidated residue
  SITE 7..14: note = Cysteines are stapled
DPASELCEWAAIHCDLV

SEQ ID NO: 58 - protein, 17 aa, synthetic construct
  SITE 1: note = Acetylated residue
  SITE 17: note = Amidated residue
DPASELCEWAAIHCDLV

SEQ ID NO: 59 - protein, 17 aa, synthetic construct
  SITE 1: note = Acetylated residue
  SITE 17: note = Amidated residue
  SITE 7..14: note = Cysteines are stapled
DPAILECHIAAWNCYEI

SEQ ID NO: 60 - protein, 17 aa, synthetic construct
  SITE 1: note = Acetylated residue
  SITE 17: note = Amidated residue
  SITE 7..14: note = Cysteines are stapled
DPATLDCHIAAWDCWDE

SEQ ID NO: 61 - protein, 17 aa, synthetic construct
  SITE 1: note = Acetylated residue
  SITE 17: note = Amidated residue
  SITE 7..14: note = Cysteines are stapled
DPAILACHLAAMDCSDW

SEQ ID NO: 62 - protein, 17 aa, synthetic construct
  SITE 1: note = Acetylated residue
  SITE 17: note = Amidated residue
DPAILACHLAAMDCSDW

SEQ ID NO: 63 - protein, 17 aa, synthetic construct
  SITE 1: note = Acetylated residue
  SITE 17: note = Amidated residue
  SITE 7..14: note = Cysteines are stapled
DPANANCILAAHECRIW

SEQ ID NO: 64 - protein, 17 aa, synthetic construct
  SITE 1: note = Acetylated residue
  SITE 17: note = Amidated residue
  SITE 7..14: note = Cysteines are stapled
DPAEVECMLAAHVCRAF

SEQ ID NO: 65 - protein, 17 aa, synthetic construct
  SITE 1: note = Acetylated residue
  SITE 17: note = Amidated residue
  SITE 7..14: note = Cysteines are stapled
DPAQDDCILAAHVCALW

SEQ ID NO: 66 - protein, 17 aa, synthetic construct
  SITE 1: note = Acetylated residue
  SITE 17: note = Amidated residue
DPAQDDCILAAHVCALW

SEQ ID NO: 67 - protein, 17 aa, synthetic construct
  SITE 1: note = Acetylated residue
  SITE 17: note = Amidated residue
  SITE 7..14: note = Cysteines are stapled
DPADWECEHAALLCHYW

SEQ ID NO: 68 - protein, 17 aa, synthetic construct
  SITE 1: note = Acetylated residue
  SITE 17: note = Amidated residue
  SITE 7..14: note = Cysteines are stapled
DPALWQCEHAALLCDVH

SEQ ID NO: 69 - protein, 17 aa, synthetic construct
  SITE 1: note = Acetylated residue
  SITE 17: note = Amidated residue
DPALWQCEHAALLCDVH

SEQ ID NO: 70 - protein, 17 aa, synthetic construct
  SITE 1: note = Acetylated residue
  SITE 17: note = Amidated residue
  SITE 7..14: note = Cysteines are stapled
DPAIIHCYEAAFFCQYI

SEQ ID NO: 71 - protein, 17 aa, synthetic construct
  SITE 1: note = Acetylated residue
  SITE 17: note = Amidated residue
  SITE 7..14: note = Cysteines are stapled
DPAVMECYEAAFICHYV

SEQ ID NO: 72 - protein, 45 aa, synthetic construct
  SITE 1: note = 5-Carboxyfluorescein beta-alanine
  SITE 45: note = Amidated residue
XDDLGANDELISFKDEGEQEEKSSENSSAERDLADVKSSLVNESE

SEQ ID NO: 73 - protein, 14 aa, synthetic construct
  SITE 1: note = Residue attached to fluorescein isothiocyanate 1x polyethylene glycol
  SITE 3: note = (S)-2-amino-2-methylhept-6-enoic acid, useful for stapling/stapled with another amino acid residue comprising an olefin group to form a staple through the olefin group
  SITE 7: note = (S)-2-amino-2-methylhept-6-enoic acid, useful for stapling/stapled with another amino acid residue comprising an olefin group to form a staple through the olefin group
  SITE 3..7: note = Residues are stapled
PQXILDXHVRRVWR

SEQ ID NO: 74 - protein, 17 aa, synthetic construct
  SITE 1: note = Acetylated residue
  SITE 17: note = Amidated residue
  SITE 7..14: note = Cysteines are stapled
DPAEWICRMAAMNCLYQ

SEQ ID NO: 75 - protein, 17 aa, synthetic construct
  SITE 1: note = Acetylated residue
  SITE 17: note = Amidated residue
DPAEWICRMAAMNCLYQ

SEQ ID NO: 76 - protein, 17 aa, synthetic construct
  SITE 1: note = Acetylated residue
  SITE 17: note = Amidated residue
  SITE 7..14: note = Cysteines are stapled
DPARDWCLYAAYDCYTA

SEQ ID NO: 77 - protein, 17 aa, synthetic construct
  SITE 1: note = Acetylated residue
  SITE 17: note = Amidated residue
DPARDWCLYAAYDCYTA

SEQ ID NO: 78 - protein, 17 aa, synthetic construct
  SITE 1: note = Acetylated residue
  SITE 17: note = Amidated residue
  SITE 7..14: note = Cysteines are stapled
DPAFTDCQLAAAVCMTY

SEQ ID NO: 79 - protein, 17 aa, synthetic construct
  SITE 1: note = Acetylated residue
  SITE 17: note = Amidated residue
  SITE 7..14: note = Cysteines are stapled
DPAIVQCAWAALYCDMQ

SEQ ID NO: 80 - protein, 20 aa, synthetic construct
  SITE 1: note = Fluorescein isothiocyanate aminohexanoic acid
  SITE 20: note = Amidated residue
XAEHEEDMYRAADEIEKEKE

SEQ ID NO: 81 - protein, 17 aa, synthetic construct
  SITE 1: note = Acetylated residue
  SITE 17: note = Amidated residue
  SITE 7..14: note = Cysteines are stapled
DPAMQRCFSAAVYCAIS

SEQ ID NO: 82 - protein, 18 aa, synthetic construct
  SITE 1: note = 5-Carboxyfluorescein beta-alanine
  SITE 18: note = Amidated residue
  SITE 8..15: note = Cysteines are stapled
XDPAMQRCFSAAVYCAIS

SEQ ID NO: 83 - protein, 10 aa, synthetic construct
  SITE 1: note = Acetylated residue
  SITE 10: note = Amidated residue
  SITE 3..10: note = Cysteines are stapled
FECLDAFFSC

SEQ ID NO: 84 - protein, 17 aa, synthetic construct
  SITE 1: note = Acetylated residue
  SITE 17: note = Amidated residue
  SITE 7..14: note = Cysteines are stapled
DPAWWVCAIAAIECSDV

SEQ ID NO: 85 - protein, 13 aa, synthetic construct
  SITE 1: note = Acetylated residue
  SITE 13: note = Amidated residue
  SITE 4..11: note = Cysteines are stapled
WWVCAIAAIECSD

SEQ ID NO: 86 - protein, 11 aa, synthetic construct
  SITE 1: note = Acetylated residue
  SITE 11: note = Amidated residue
  SITE 3..10: note = Cysteines are stapled
PDCHIRAYVCH

SEQ ID NO: 87 - protein, 17 aa, synthetic construct
  SITE 1: note = Acetylated residue
  SITE 17: note = Amidated residue
  SITE 7..14: note = Cysteines are stapled
DPANQDCHVAAWHCWQR

SEQ ID NO: 88 - protein, 11 aa, synthetic construct
  SITE 1: note = Acetylated residue
  SITE 11: note = Amidated residue
  SITE 3..10: note = Cysteines are stapled
PECHIEAYWCI

SEQ ID NO: 89 - protein, 17 aa, synthetic construct
  SITE 1: note = Acetylated residue
  SITE 17: note = Amidated residue
  SITE 7..14: note = Cysteines are stapled
DPAAADCQWAAFLCRVY

SEQ ID NO: 90 - protein, 17 aa, synthetic construct
  SITE 1: note = Acetylated residue
  SITE 17: note = Amidated residue
  SITE 7..14: note = Cysteines are stapled
DPALWQCVFAARSCYEE

SEQ ID NO: 91 - protein, 17 aa, synthetic construct
  SITE 1: note = Acetylated residue
  SITE 17: note = Amidated residue
  SITE 7..14: note = Cysteines are stapled
DPALAQCVFAARSCYEE

SEQ ID NO: 92 - protein, 17 aa, synthetic construct
  SITE 1: note = Acetylated residue
  SITE 17: note = Amidated residue
  SITE 7..14: note = Cysteines are stapled
DPALWMCVFAARQCYES

SEQ ID NO: 93 - protein, 17 aa, synthetic construct
  SITE 1: note = Acetylated residue
  SITE 17: note = Amidated residue
  SITE 7..14: note = Cysteines are stapled
DPALWQCVFAARYCYEE

SEQ ID NO: 94 - protein, 14 aa, synthetic construct
  SITE 1: note = 5-Carboxyfluorescein modified residue
  SITE 14: note = Amidated residue
QRFARKGALRQKNV

SEQ ID NO: 95 - protein, 10 aa, synthetic construct
  SITE 1: note = 5-Carboxyfluorescein modified D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
  SITE 8: note = D-amino acid
  SITE 9: note = D-amino acid
  SITE 10: note = Amidated D-amino acid
EQKLISEEDL

SEQ ID NO: 96 - protein, 9 aa, synthetic construct
  SITE 1: note = 5-Carboxyfluorescein modified D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
  SITE 8: note = D-amino acid
  SITE 9: note = Amidated D-amino acid
YPYDVPDYA

SEQ ID NO: 97 - protein, 17 aa, synthetic construct
  SITE 1: note = Acetylated 5-Carboxyfluorescein modified residue
  SITE 8: note = (R)-2-amino-2-methyldec-9-enoic acid, useful for stapling/stapled with another amino acid residue comprising an olefin group to form a staple through the olefin group
  SITE 15: note = (S)-2-amino-2-methylhept-6-enoic acid, useful for stapling/stapled with another amino acid residue comprising an olefin group to form a staple through the olefin group
  SITE 17: note = Amidated residue
  SITE 8..15: note = Residues are stapled
KQSQQTFXNLWRLLXQN

SEQ ID NO: 98 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 10: note = (S)-2-amino-3-cyclobutylpropanoic acid
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
LTFXEYWAQXXSAA

SEQ ID NO: 99 - protein, 14 aa, synthetic construct
  SITE 1: note = Acetylated residue
  SITE 2: note = (S)-2-aminohexanoic acid
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
VXEXYEAAFIXHYV

SEQ ID NO: 100 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
ILQXYEAAFSXHYQ

SEQ ID NO: 101 - protein, 14 aa, synthetic construct
  SITE 1: note = Acetylated residue
  SITE 2: note = (S)-2-aminohexanoic acid
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
VXQXYEAAFIXHYV

SEQ ID NO: 102 - protein, 14 aa, synthetic construct
  SITE 1: note = Acetylated residue
  SITE 2: note = (S)-2-aminohexanoic acid
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
VXEXYQAAFIXHYV

SEQ ID NO: 103 - protein, 14 aa, synthetic construct
  SITE 1: note = Acetylated residue
  SITE 2: note = (S)-2-aminohexanoic acid
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
VXQXYQAAFIXHYV

SEQ ID NO: 104 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
IIHXYEAAFFXQYI

SEQ ID NO: 105 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated (S)-2-aminohexanoic acid
  SITE 4..11: note = Residues are stapled
ILEXYEAAFEXQYX

SEQ ID NO: 106 - protein, 14 aa, synthetic construct
  SITE 1: note = Acetylated residue
  SITE 2: note = (S)-2-aminohexanoic acid
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
IXAXYQAAFWXQYN

SEQ ID NO: 107 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
ILTXYEAAFTXQYQ

SEQ ID NO: 108 - protein, 17 aa, synthetic construct
DPAVMECYEAAFICHYV

SEQ ID NO: 109 - protein, 17 aa, synthetic construct
DPAILQCYEAAFSCHYQ

SEQ ID NO: 110 - protein, 17 aa, synthetic construct
DPAIIHCYEAAFFCQYI

SEQ ID NO: 111 - protein, 17 aa, synthetic construct
DPAILECYEAAFECQYM

SEQ ID NO: 112 - protein, 17 aa, synthetic construct
DPAIMACYQAAFWCQYN

SEQ ID NO: 113 - protein, 17 aa, synthetic construct
DPAILTCYEAAFTCQYQ

SEQ ID NO: 114 - protein, 17 aa, synthetic construct
DPAEHDCMLAAHFCRHF

SEQ ID NO: 115 - protein, 17 aa, synthetic construct
DPAQDDCILAAHVCALW

SEQ ID NO: 116 - protein, 17 aa, synthetic construct
DPAVEDCVLAAHVCAWF

SEQ ID NO: 117 - protein, 17 aa, synthetic construct
DPAEAECILAAHICNIS

SEQ ID NO: 118 - protein, 17 aa, synthetic construct
DPAEVDCIMAAHICVWD

SEQ ID NO: 119 - protein, 17 aa, synthetic construct
DPADNECILAAHICTLA

SEQ ID NO: 120 - protein, 17 aa, synthetic construct
DPAQQDCTLAAHVCRVF

SEQ ID NO: 121 - protein, 17 aa, synthetic construct
DPAEADCMMAAHICSNI

SEQ ID NO: 122 - protein, 17 aa, synthetic construct
DPADYECVIAAQVCQHF

SEQ ID NO: 123 - protein, 17 aa, synthetic construct
DPANANCILAAHECRIW

SEQ ID NO: 124 - protein, 17 aa, synthetic construct
DPAFESCFLAAHICYWE

SEQ ID NO: 125 - protein, 17 aa, synthetic construct
DPAEAICILAAHECWWD

SEQ ID NO: 126 - protein, 17 aa, synthetic construct
DPAEVECMLAAHVCRAF

SEQ ID NO: 127 - protein, 17 aa, synthetic construct
DPADYDCILAAHICTNI

SEQ ID NO: 128 - protein, 17 aa, synthetic construct
DPADTECILAAHFCHYF

SEQ ID NO: 129 - protein, 17 aa, synthetic construct
DPAVDECIIAAHICHIW

SEQ ID NO: 130 - protein, 17 aa, synthetic construct
DPAESDCILAAYICYVE

SEQ ID NO: 131 - protein, 17 aa, synthetic construct
DPADEDCILAAHVCRHF

SEQ ID NO: 132 - protein, 17 aa, synthetic construct
DPAADDCVLAAHVCAYF

SEQ ID NO: 133 - protein, 17 aa, synthetic construct
DPARHECILAAHICEWE

SEQ ID NO: 134 - protein, 17 aa, synthetic construct
DPAILDCQIAAIECEFA

SEQ ID NO: 135 - protein, 17 aa, synthetic construct
DPAILDCHIAAIQCFHD

SEQ ID NO: 136 - protein, 17 aa, synthetic construct
DPAIFNCTMAALECTQI

SEQ ID NO: 137 - protein, 17 aa, synthetic construct
DPAILECHIAAWNCYEI

SEQ ID NO: 138 - protein, 17 aa, synthetic construct
DPAHLMCHYAAWDCRDE

SEQ ID NO: 139 - protein, 17 aa, synthetic construct
DPAILDCQLAAMDCSTW

SEQ ID NO: 140 - protein, 17 aa, synthetic construct
DPAFLTCHIAAWACSEQ

SEQ ID NO: 141 - protein, 17 aa, synthetic construct
DPAFLDCHIAAWTCNEF

SEQ ID NO: 142 - protein, 17 aa, synthetic construct
DPAILDCQLAAIDCVYT

SEQ ID NO: 143 - protein, 17 aa, synthetic construct
DPAILDCSIAAIDCAYS

SEQ ID NO: 144 - protein, 17 aa, synthetic construct
DPAILECHMAAFECYIE

SEQ ID NO: 145 - protein, 17 aa, synthetic construct
DPAVFECSLAAIDCLQI

SEQ ID NO: 146 - protein, 17 aa, synthetic construct
DPAVLECHIAAWECRMD

SEQ ID NO: 147 - protein, 17 aa, synthetic construct
DPAILECHVAALDCQMW

SEQ ID NO: 148 - protein, 17 aa, synthetic construct
DPAILNCHMAAWDCNFY

SEQ ID NO: 149 - protein, 17 aa, synthetic construct
DPATLDCHIAAWDCWDE

SEQ ID NO: 150 - protein, 17 aa, synthetic construct
DPAILACHLAAMDCSDW

SEQ ID NO: 151 - protein, 17 aa, synthetic construct
DPAVLECHIAAWECNHT

SEQ ID NO: 152 - protein, 17 aa, synthetic construct
DPAVLDCHIAALDCHAH

SEQ ID NO: 153 - protein, 17 aa, synthetic construct
DPAILNCHVAAWNCHSF

SEQ ID NO: 154 - protein, 17 aa, synthetic construct
DPAELACEWAAIHCELV

SEQ ID NO: 155 - protein, 17 aa, synthetic construct
DPASELCEWAAIHCDLV

SEQ ID NO: 156 - protein, 17 aa, synthetic construct
DPADDFCMWAAEHCELL

SEQ ID NO: 157 - protein, 17 aa, synthetic construct
DPAWQNCTWAAFHCELL

SEQ ID NO: 158 - protein, 17 aa, synthetic construct
DPAQNICEWAALHCDLI

SEQ ID NO: 159 - protein, 17 aa, synthetic construct
DPAEFYCEMAAVHCELL

SEQ ID NO: 160 - protein, 17 aa, synthetic construct
DPAVHRCFWAAEHCDLI

SEQ ID NO: 161 - protein, 17 aa, synthetic construct
DPAWILCDWAAMHCELV

SEQ ID NO: 162 - protein, 17 aa, synthetic construct
DPAMIQCQWAAEHCELI

SEQ ID NO: 163 - protein, 17 aa, synthetic construct
DPAIADCDWAAEHCDLL

SEQ ID NO: 164 - protein, 17 aa, synthetic construct
DPAVQDCEWAAIYCELI

SEQ ID NO: 165 - protein, 17 aa, synthetic construct
DPAWSDCIWAAEHCQLI

SEQ ID NO: 166 - protein, 17 aa, synthetic construct
DPAMSQCNWAAVHCQLL

SEQ ID NO: 167 - protein, 17 aa, synthetic construct
DPANVECEWAAIHCSLV

SEQ ID NO: 168 - protein, 17 aa, synthetic construct
DPAQSDCDWAAEHCNLI

SEQ ID NO: 169 - protein, 17 aa, synthetic construct
DPAAQRCTWAAIHCELI

SEQ ID NO: 170 - protein, 17 aa, synthetic construct
DPAYTYCQWAAEHCSLL

SEQ ID NO: 171 - protein, 17 aa, synthetic construct
DPAHWSCEWAAEHCRLI

SEQ ID NO: 172 - protein, 17 aa, synthetic construct
DPAENTCSWAAEHCALV

SEQ ID NO: 173 - protein, 17 aa, synthetic construct
DPATWHCDWAAEHCELL

SEQ ID NO: 174 - protein, 17 aa, synthetic construct
DPALWNCDHAALLCDHQ

SEQ ID NO: 175 - protein, 17 aa, synthetic construct
DPAEWDCMHAALICEVH

SEQ ID NO: 176 - protein, 17 aa, synthetic construct
DPASWRCEHAALNCQID

SEQ ID NO: 177 - protein, 17 aa, synthetic construct
DPAWWQCDHAALICIAE

SEQ ID NO: 178 - protein, 17 aa, synthetic construct
DPAEWQCEHAALLCEQQ

SEQ ID NO: 179 - protein, 17 aa, synthetic construct
DPAQWACEHAALLCSMD

SEQ ID NO: 180 - protein, 17 aa, synthetic construct
DPALWQCEHAALLCDVH

SEQ ID NO: 181 - protein, 17 aa, synthetic construct
DPADWICEHAALTCQEF

SEQ ID NO: 182 - protein, 17 aa, synthetic construct
DPAHWVCEHAALNCDQN

SEQ ID NO: 183 - protein, 17 aa, synthetic construct
DPADWECEHAALLCHYW

SEQ ID NO: 184 - protein, 17 aa, synthetic construct
DPAEWICEHAALLCSVL

SEQ ID NO: 185 - protein, 17 aa, synthetic construct
DPAEWACEHAALTCWDQ

SEQ ID NO: 186 - protein, 17 aa, synthetic construct
DPASWDCDHAALLCTLD

SEQ ID NO: 187 - protein, 17 aa, synthetic construct
DPAQFYCEHAALLCRWE

SEQ ID NO: 188 - protein, 17 aa, synthetic construct
DPAHWRCEHAALLCWYN

SEQ ID NO: 189 - protein, 17 aa, synthetic construct
DPAEWYCEHAALVCTNH

SEQ ID NO: 190 - protein, 17 aa, synthetic construct
DPAMWICEHAALNCEIR

SEQ ID NO: 191 - protein, 17 aa, synthetic construct
DPAYWDCTHAALLCNLD

SEQ ID NO: 192 - protein, 17 aa, synthetic construct
DPAHWHCEHAALLCASV

SEQ ID NO: 193 - protein, 17 aa, synthetic construct
DPAYWVCEHAALLCTDY

SEQ ID NO: 194 - protein, 17 aa, synthetic construct
DPATIRCYEAAFMCQYW

SEQ ID NO: 195 - protein, 17 aa, synthetic construct
DPAIMACYEAAYLCDYI

SEQ ID NO: 196 - protein, 17 aa, synthetic construct
DPAIIRCYEAAFYCQNY

SEQ ID NO: 197 - protein, 17 aa, synthetic construct
DPAVIQCYQAAFHCDYQ

SEQ ID NO: 198 - protein, 17 aa, synthetic construct
DPAEIICYEAAFLCQYQ

SEQ ID NO: 199 - protein, 17 aa, synthetic construct
DPAALSCYQAAFHCQYY

SEQ ID NO: 200 - protein, 17 aa, synthetic construct
DPARMTCYEAAFRCQYY

SEQ ID NO: 201 - protein, 17 aa, synthetic construct
DPAVVECYEAAYTCQYI

SEQ ID NO: 202 - protein, 17 aa, synthetic construct
DPAVLQCYEAAFICEYM

SEQ ID NO: 203 - protein, 17 aa, synthetic construct
DPAVMACYEAAFQCAYW

SEQ ID NO: 204 - protein, 17 aa, synthetic construct
DPAVIACYDAAFVCEYF

SEQ ID NO: 205 - protein, 17 aa, synthetic construct
DPAVFMCYEAAFECQYF

SEQ ID NO: 206 - protein, 17 aa, synthetic construct
DPAIIHCYQAAFQCQYM

SEQ ID NO: 207 - protein, 17 aa, synthetic construct
DPAMVTCYEAAYVCQYF

SEQ ID NO: 208 - protein, 17 aa, synthetic construct
DPAIVQCAWAALYCDMQ

SEQ ID NO: 209 - protein, 17 aa, synthetic construct
DPALEDCQWAALYCDEY

SEQ ID NO: 210 - protein, 17 aa, synthetic construct
DPAYEDCAWAALYCDMT

SEQ ID NO: 211 - protein, 17 aa, synthetic construct
DPAYYTCDWAAIYCDYH

SEQ ID NO: 212 - protein, 17 aa, synthetic construct
DPAYRDCDWAALYCDDF

SEQ ID NO: 213 - protein, 17 aa, synthetic construct
DPAWTQCDWAALYCEDQ

SEQ ID NO: 214 - protein, 17 aa, synthetic construct
DPAEDECDWAALYCDMQ

SEQ ID NO: 215 - protein, 17 aa, synthetic construct
DPAYEDCADAAMYCDMD

SEQ ID NO: 216 - protein, 17 aa, synthetic construct
DPAWTDCDWAALYCEFQ

SEQ ID NO: 217 - protein, 17 aa, synthetic construct
DPAWTDCDWAALYCDES

SEQ ID NO: 218 - protein, 17 aa, synthetic construct
DPALVTCTWAAIYCDDF

SEQ ID NO: 219 - protein, 17 aa, synthetic construct
DPAYTLCSWAAMYCDSM

SEQ ID NO: 220 - protein, 17 aa, synthetic construct
DPAYSSCSWAAIYCNWT

SEQ ID NO: 221 - protein, 17 aa, synthetic construct
DPAADDCEWAALYCSFH

SEQ ID NO: 222 - protein, 17 aa, synthetic construct
DPAWNDCLYAAETCISW

SEQ ID NO: 223 - protein, 17 aa, synthetic construct
DPAWWNCLYAANTCVFD

SEQ ID NO: 224 - protein, 17 aa, synthetic construct
DPAQHECIYAAWDCDML

SEQ ID NO: 225 - protein, 17 aa, synthetic construct
DPAWWDCMYAASHCDTV

SEQ ID NO: 226 - protein, 17 aa, synthetic construct
DPAWMQCMYAAWDCNIE

SEQ ID NO: 227 - protein, 17 aa, synthetic construct
DPAYWDCMYAAFYCDDI

SEQ ID NO: 228 - protein, 17 aa, synthetic construct
DPAYWDCLYAAADCAFY

SEQ ID NO: 229 - protein, 17 aa, synthetic construct
DPAYWDCLYAAWSCDIL

SEQ ID NO: 230 - protein, 17 aa, synthetic construct
DPAWWSCMYAAWTCDDT

SEQ ID NO: 231 - protein, 17 aa, synthetic construct
DPAWLDCIYAAYDCVTD

SEQ ID NO: 232 - protein, 17 aa, synthetic construct
DPAYWQCIYAAWDCDQM

SEQ ID NO: 233 - protein, 17 aa, synthetic construct
DPAWWNCLYAAYECNHA

SEQ ID NO: 234 - protein, 17 aa, synthetic construct
DPALWDCLYAADACITS

SEQ ID NO: 235 - protein, 17 aa, synthetic construct
DPAFWECMYAASDCDAS

SEQ ID NO: 236 - protein, 17 aa, synthetic construct
DPAFWACLYAAEECVVD

SEQ ID NO: 237 - protein, 17 aa, synthetic construct
DPAWMDCLYAAYDCQHM

SEQ ID NO: 238 - protein, 17 aa, synthetic construct
DPAYWHCLYAAASCDDI

SEQ ID NO: 239 - protein, 17 aa, synthetic construct
DPAEWDCLYAAFDCFAN

SEQ ID NO: 240 - protein, 17 aa, synthetic construct
DPADWDCMYAAQDCFED

SEQ ID NO: 241 - protein, 17 aa, synthetic construct
DPAWWNCLYAADECWSW

SEQ ID NO: 242 - protein, 17 aa, synthetic construct
DPAYDWCMYAAHECVDQ

SEQ ID NO: 243 - protein, 17 aa, synthetic construct
DPALDYCLYAAYDCMYA

SEQ ID NO: 244 - protein, 17 aa, synthetic construct
DPATWWCLYAAEDCVNI

SEQ ID NO: 245 - protein, 17 aa, synthetic construct
DPAYDWCLYAAYQCDQN

SEQ ID NO: 246 - protein, 17 aa, synthetic construct
DPAWEWCLYAAAECIED

SEQ ID NO: 247 - protein, 17 aa, synthetic construct
DPAEDWCMYAAWDCQME

SEQ ID NO: 248 - protein, 17 aa, synthetic construct
DPAQDWCMYAAYECDAH

SEQ ID NO: 249 - protein, 17 aa, synthetic construct
DPAWDYCMYAAYDCNHS

SEQ ID NO: 250 - protein, 17 aa, synthetic construct
DPARDWCLYAAYDCYTA

SEQ ID NO: 251 - protein, 17 aa, synthetic construct
DPAHDWCLYAAYDCRVY

SEQ ID NO: 252 - protein, 17 aa, synthetic construct
DPAWEHCLYAAYDCTHE

SEQ ID NO: 253 - protein, 17 aa, synthetic construct
DPAYDWCMYAAYNCVED

SEQ ID NO: 254 - protein, 17 aa, synthetic construct
DPAWESCMYAAYDCDNE

SEQ ID NO: 255 - protein, 17 aa, synthetic construct
DPAEQFCLYAAYDCYSE

SEQ ID NO: 256 - protein, 17 aa, synthetic construct
DPAYDWCLYAAEYCHTD

SEQ ID NO: 257 - protein, 17 aa, synthetic construct
DPAYDYCLYAAQDCVWW

SEQ ID NO: 258 - protein, 17 aa, synthetic construct
DPAFDWCLYAADDCAET

SEQ ID NO: 259 - protein, 17 aa, synthetic construct
DPAHTWCMYAAFDCFES

SEQ ID NO: 260 - protein, 17 aa, synthetic construct
DPAYDYCLYAAYDCTQT

SEQ ID NO: 261 - protein, 17 aa, synthetic construct
DPAQYQCDIAAQVCSTY

SEQ ID NO: 262 - protein, 17 aa, synthetic construct
DPAFAQCEVAAMVCTTY

SEQ ID NO: 263 - protein, 17 aa, synthetic construct
DPAIQECLLAAATCWTY

SEQ ID NO: 264 - protein, 17 aa, synthetic construct
DPAHANCQLAADVCSTY

SEQ ID NO: 265 - protein, 17 aa, synthetic construct
DPAWQECVIAALVCDTY

SEQ ID NO: 266 - protein, 17 aa, synthetic construct
DPAIYQCSLAADICSTY

SEQ ID NO: 267 - protein, 17 aa, synthetic construct
DPAIWECVSAAAVCMTY

SEQ ID NO: 268 - protein, 17 aa, synthetic construct
DPAIQECYLAAQVCTTY

SEQ ID NO: 269 - protein, 17 aa, synthetic construct
DPAAWECHIAAFVCSTY

SEQ ID NO: 270 - protein, 17 aa, synthetic construct
DPAAEDCHIAAMVCMTY

SEQ ID NO: 271 - protein, 17 aa, synthetic construct
DPAIRECMLAALVCSTY

SEQ ID NO: 272 - protein, 17 aa, synthetic construct
DPAFTDCQLAAAVCMTY

SEQ ID NO: 273 - protein, 17 aa, synthetic construct
DPAADQCDIAAFVCSTY

SEQ ID NO: 274 - protein, 17 aa, synthetic construct
DPAFMDCVFAAAVCATY

SEQ ID NO: 275 - protein, 17 aa, synthetic construct
DPALSHCTVAAMVCSTY

SEQ ID NO: 276 - protein, 17 aa, synthetic construct
DPANFTCRVAADVCSTY

SEQ ID NO: 277 - protein, 17 aa, synthetic construct
DPAIAQCMLAAQVCSTY

SEQ ID NO: 278 - protein, 17 aa, synthetic construct
DPAMQDCQLAALVCTTY

SEQ ID NO: 279 - protein, 17 aa, synthetic construct
DPAFHDCQFAAAVCSTY

SEQ ID NO: 280 - protein, 17 aa, synthetic construct
DPAMIDCFLAAQVCATY

SEQ ID NO: 281 - protein, 17 aa, synthetic construct
DPAFQFCVEAAVACSYY

SEQ ID NO: 282 - protein, 17 aa, synthetic construct
DPAEDECQDAAILCSYN

SEQ ID NO: 283 - protein, 17 aa, synthetic construct
DPALRDCLDAAIMCTYT

SEQ ID NO: 284 - protein, 17 aa, synthetic construct
DPAQYQCWNAAMSCSYS

SEQ ID NO: 285 - protein, 17 aa, synthetic construct
DPAQMECIDAAIMCSYT

SEQ ID NO: 286 - protein, 17 aa, synthetic construct
DPAQDQCISAAVLCTYT

SEQ ID NO: 287 - protein, 17 aa, synthetic construct
DPAQEACWDAAIMCTYR

SEQ ID NO: 288 - protein, 17 aa, synthetic construct
DPAEEYCLEAAILCTYD

SEQ ID NO: 289 - protein, 17 aa, synthetic construct
DPAMQECMDAAIMCTYA

SEQ ID NO: 290 - protein, 17 aa, synthetic construct
DPAMEWCWDAAVFCTYS

SEQ ID NO: 291 - protein, 17 aa, synthetic construct
DPAVVDCWDAAVQCTYY

SEQ ID NO: 292 - protein, 17 aa, synthetic construct
DPAIDACYDAAVMCTYV

SEQ ID NO: 293 - protein, 17 aa, synthetic construct
DPASHMCLEAAVMCTYD

SEQ ID NO: 294 - protein, 17 aa, synthetic construct
DPATEACFDAAIECTYY

SEQ ID NO: 295 - protein, 17 aa, synthetic construct
DPAYDLCLNAAVACTYV

SEQ ID NO: 296 - protein, 17 aa, synthetic construct
DPAYSLCISAAVECTYY

SEQ ID NO: 297 - protein, 17 aa, synthetic construct
DPARRECWDAAILCTYY

SEQ ID NO: 298 - protein, 17 aa, synthetic construct
DPASEWCLDAAVLCTYS

SEQ ID NO: 299 - protein, 17 aa, synthetic construct
DPALEICWDAAIQCSYV

SEQ ID NO: 300 - protein, 17 aa, synthetic construct
DPAQEECFEAAVFCSYY

SEQ ID NO: 301 - protein, 17 aa, synthetic construct
DPAMQRCFSAAVYCAIS

SEQ ID NO: 302 - protein, 17 aa, synthetic construct
DPAQMSCFSAAVYCRLF

SEQ ID NO: 303 - protein, 17 aa, synthetic construct
DPAQRQCLSAAVWCATF

SEQ ID NO: 304 - protein, 17 aa, synthetic construct
DPAALACLSAAVYCEMY

SEQ ID NO: 305 - protein, 17 aa, synthetic construct
DPALQSCLSAAVYCTVS

SEQ ID NO: 306 - protein, 17 aa, synthetic construct
DPAVQSCLSAAVYCAFR

SEQ ID NO: 307 - protein, 17 aa, synthetic construct
DPAQMFCYSAAVYCAAF

SEQ ID NO: 308 - protein, 17 aa, synthetic construct
DPAASQCFSAAVYCAWI

SEQ ID NO: 309 - protein, 17 aa, synthetic construct
DPAEQRCLSAAVYCAWH

SEQ ID NO: 310 - protein, 17 aa, synthetic construct
DPAHVRCTSAALWCNIF

SEQ ID NO: 311 - protein, 17 aa, synthetic construct
DPAAQACLSAAVFCAWY

SEQ ID NO: 312 - protein, 17 aa, synthetic construct
DPALRTCLSAAVFCAVA

SEQ ID NO: 313 - protein, 17 aa, synthetic construct
DPASASCLSAAIYCSMF

SEQ ID NO: 314 - protein, 17 aa, synthetic construct
DPAQLNCLSAAMYCALY

SEQ ID NO: 315 - protein, 17 aa, synthetic construct
DPAQRQCLSAAVFCATL

SEQ ID NO: 316 - protein, 17 aa, synthetic construct
DPALTNCFSAAVYCAMF

SEQ ID NO: 317 - protein, 17 aa, synthetic construct
DPASMVCLSAAVYCAMH

SEQ ID NO: 318 - protein, 17 aa, synthetic construct
DPAQQICLSAAVFCSIF

SEQ ID NO: 319 - protein, 17 aa, synthetic construct
DPAMIQCLSAAVYCVHY

SEQ ID NO: 320 - protein, 17 aa, synthetic construct
DPAQRDCFSAAVYCSFL

SEQ ID NO: 321 - protein, 17 aa, synthetic construct
DPAFSQCVYAAFFCREI

SEQ ID NO: 322 - protein, 17 aa, synthetic construct
DPAFSTCVYAAVFCQNT

SEQ ID NO: 323 - protein, 17 aa, synthetic construct
DPAFSTCVYAAIICNDT

SEQ ID NO: 324 - protein, 17 aa, synthetic construct
DPAFSTCIYAAVMCMDQ

SEQ ID NO: 325 - protein, 17 aa, synthetic construct
DPAFSECVYAAIYCVHS

SEQ ID NO: 326 - protein, 17 aa, synthetic construct
DPAFSQCVYAAVVCEVQ

SEQ ID NO: 327 - protein, 17 aa, synthetic construct
DPAFSRCVYAAVACEDH

SEQ ID NO: 328 - protein, 17 aa, synthetic construct
DPAFSLCVYAALFCEQQ

SEQ ID NO: 329 - protein, 17 aa, synthetic construct
DPAFSACVYAAYMCNQD

SEQ ID NO: 330 - protein, 17 aa, synthetic construct
DPAFSQCVYAAWHCESL

SEQ ID NO: 331 - protein, 17 aa, synthetic construct
DPALSTCVYAAVMCQDV

SEQ ID NO: 332 - protein, 17 aa, synthetic construct
DPAFSMCVYAAVTCVST

SEQ ID NO: 333 - protein, 17 aa, synthetic construct
DPAFSRCVYAAFTCQTF

SEQ ID NO: 334 - protein, 17 aa, synthetic construct
DPAFSECVYAAFSCSWI

SEQ ID NO: 335 - protein, 17 aa, synthetic construct
DPAFFTCVYAAVTCEMT

SEQ ID NO: 336 - protein, 17 aa, synthetic construct
DPAFYECVYAAVTCTQF

SEQ ID NO: 337 - protein, 17 aa, synthetic construct
DPALSQCVYAAVFCQNL

SEQ ID NO: 338 - protein, 17 aa, synthetic construct
DPAFSLCVYAAVECDHA

SEQ ID NO: 339 - protein, 17 aa, synthetic construct
DPAFSQCIYAAFQCDMQ

SEQ ID NO: 340 - protein, 17 aa, synthetic construct
DPAFSECVYAATMCLNH

SEQ ID NO: 341 - protein, 17 aa, synthetic construct
DPAYEHCVAAAYWCYLL

SEQ ID NO: 342 - protein, 17 aa, synthetic construct
DPAREHCDSAAIFCYIL

SEQ ID NO: 343 - protein, 17 aa, synthetic construct
DPAREHCAAAATWCQLL

SEQ ID NO: 344 - protein, 17 aa, synthetic construct
DPAHEHCTAAASWCYIL

SEQ ID NO: 345 - protein, 17 aa, synthetic construct
DPAQNHCDSAAVFCYIL

SEQ ID NO: 346 - protein, 17 aa, synthetic construct
DPAHVHCQAAAQWCVLL

SEQ ID NO: 347 - protein, 17 aa, synthetic construct
DPAREHCDSAAAWCWVL

SEQ ID NO: 348 - protein, 17 aa, synthetic construct
DPAITHCTAAAMWCHLL

SEQ ID NO: 349 - protein, 17 aa, synthetic construct
DPASSHCQSAAIWCYLM

SEQ ID NO: 350 - protein, 17 aa, synthetic construct
DPASIHCAAAALWCDIL

SEQ ID NO: 351 - protein, 17 aa, synthetic construct
DPANEHCIAAAWWCQIL

SEQ ID NO: 352 - protein, 17 aa, synthetic construct
DPALTHCTAAAYWCYVM

SEQ ID NO: 353 - protein, 17 aa, synthetic construct
DPALQHCAAAASWCWLM

SEQ ID NO: 354 - protein, 17 aa, synthetic construct
DPAHHHCQAAAAWCWVM

SEQ ID NO: 355 - protein, 17 aa, synthetic construct
DPATQHCLAAAMWCHLI

SEQ ID NO: 356 - protein, 17 aa, synthetic construct
DPAHEHCTSAAAWCMLL

SEQ ID NO: 357 - protein, 17 aa, synthetic construct
DPAQQHCSAAAAWCVVL

SEQ ID NO: 358 - protein, 17 aa, synthetic construct
DPAEYHCDAAAVWCYLM

SEQ ID NO: 359 - protein, 17 aa, synthetic construct
DPAQVHCRSAAAWCILM

SEQ ID NO: 360 - protein, 17 aa, synthetic construct
DPARYHCEAAAAWCELL

SEQ ID NO: 361 - protein, 17 aa, synthetic construct
DPADLICQLAAANCIYL

SEQ ID NO: 362 - protein, 17 aa, synthetic construct
DPAQWVCHMAAMNCLYQ

SEQ ID NO: 363 - protein, 17 aa, synthetic construct
DPAEWLCQMAAHNCIYL

SEQ ID NO: 364 - protein, 17 aa, synthetic construct
DPAMYICEMAAANCLYQ

SEQ ID NO: 365 - protein, 17 aa, synthetic construct
DPADYICYLAANNCLYH

SEQ ID NO: 366 - protein, 17 aa, synthetic construct
DPAEWICRMAAMNCLYQ

SEQ ID NO: 367 - protein, 17 aa, synthetic construct
DPAAFVCEMAANDCLYT

SEQ ID NO: 368 - protein, 17 aa, synthetic construct
DPATLVCTMAANNCIYR

SEQ ID NO: 369 - protein, 17 aa, synthetic construct
DPARWVCELAAQNCLYR

SEQ ID NO: 370 - protein, 17 aa, synthetic construct
DPAEWICEMAAHNCLYM

SEQ ID NO: 371 - protein, 17 aa, synthetic construct
DPASIICMLAADNCLYE

SEQ ID NO: 372 - protein, 17 aa, synthetic construct
DPADLLCHMAAQNCLYR

SEQ ID NO: 373 - protein, 17 aa, synthetic construct
DPAAYLCDMAAHNCLYL

SEQ ID NO: 374 - protein, 17 aa, synthetic construct
DPAHILCEMAARNCLYN

SEQ ID NO: 375 - protein, 17 aa, synthetic construct
DPADWTCVLAAHNCLYT

SEQ ID NO: 376 - protein, 17 aa, synthetic construct
DPAWWVCSIAAIECFSS

SEQ ID NO: 377 - protein, 17 aa, synthetic construct
DPAWWMCTIAAIECETT

SEQ ID NO: 378 - protein, 17 aa, synthetic construct
DPAWWRCVVAALDCVDY

SEQ ID NO: 379 - protein, 17 aa, synthetic construct
DPAWWHCSIAALECHTT

SEQ ID NO: 380 - protein, 17 aa, synthetic construct
DPAWWRCSVAAIDCHWN

SEQ ID NO: 381 - protein, 17 aa, synthetic construct
DPAWWECIVAALECSDR

SEQ ID NO: 382 - protein, 17 aa, synthetic construct
DPAWWHCVIAALDCETQ

SEQ ID NO: 383 - protein, 17 aa, synthetic construct
DPAWYECVVAAIECHAY

SEQ ID NO: 384 - protein, 17 aa, synthetic construct
DPAWWVCAIAAMDCHEQ

SEQ ID NO: 385 - protein, 17 aa, synthetic construct
DPAWWSCVIAAMECQDI

SEQ ID NO: 386 - protein, 17 aa, synthetic construct
DPAWWTCSVAAIQCWDS

SEQ ID NO: 387 - protein, 17 aa, synthetic construct
DPAWWRCSIAAVDCEMQ

SEQ ID NO: 388 - protein, 17 aa, synthetic construct
DPAWWRCSIAAMECHHD

SEQ ID NO: 389 - protein, 17 aa, synthetic construct
DPAWYACIVAAVDCEHS

SEQ ID NO: 390 - protein, 17 aa, synthetic construct
DPAWWVCAIAAIECSDV

SEQ ID NO: 391 - protein, 17 aa, synthetic construct
DPAWWVCAVAAIECQEY

SEQ ID NO: 392 - protein, 17 aa, synthetic construct
DPAWWTCIIAALECEEQ

SEQ ID NO: 393 - protein, 17 aa, synthetic construct
DPAWYECIVAAIECVEY

SEQ ID NO: 394 - protein, 10 aa, synthetic construct
CLDAFFVCEM

SEQ ID NO: 395 - protein, 11 aa, synthetic construct
PCLSAYFSCWE

SEQ ID NO: 396 - protein, 11 aa, synthetic construct
PECLEAFFDCR

SEQ ID NO: 397 - protein, 10 aa, synthetic construct
MACVDAFFSC

SEQ ID NO: 398 - protein, 11 aa, synthetic construct
PCLDAFFYCVT

SEQ ID NO: 399 - protein, 11 aa, synthetic construct
PCLEAFFSCVY

SEQ ID NO: 400 - protein, 10 aa, synthetic construct
ECLSAFFTCV

SEQ ID NO: 401 - protein, 11 aa, synthetic construct
PMACLSAFFTC

SEQ ID NO: 402 - protein, 10 aa, synthetic construct
FECLDAFFSC

SEQ ID NO: 403 - protein, 10 aa, synthetic construct
CLDAFFACER

SEQ ID NO: 404 - protein, 11 aa, synthetic construct
PECLVAFFSCV

SEQ ID NO: 405 - protein, 11 aa, synthetic construct
PSCLSAFFSCY

SEQ ID NO: 406 - protein, 11 aa, synthetic construct
PCMDAFFSCVW

SEQ ID NO: 407 - protein, 11 aa, synthetic construct
PFECLSAFFSC

SEQ ID NO: 408 - protein, 11 aa, synthetic construct
PTCLNAFFACE

SEQ ID NO: 409 - protein, 10 aa, synthetic construct
YECLEAFFNC

SEQ ID NO: 410 - protein, 11 aa, synthetic construct
PACVSAFFACL

SEQ ID NO: 411 - protein, 10 aa, synthetic construct
CLSAFFMCSS

SEQ ID NO: 412 - protein, 10 aa, synthetic construct
WSCLSAYFSC

SEQ ID NO: 413 - protein, 10 aa, synthetic construct
CLSAFFSCTY

SEQ ID NO: 414 - protein, 17 aa, synthetic construct
DPAYYDCHIAAYFCRTQ

SEQ ID NO: 415 - protein, 17 aa, synthetic construct
DPARADCHVAAYWCYHR

SEQ ID NO: 416 - protein, 17 aa, synthetic construct
DPASYDCHVAAYFCNFA

SEQ ID NO: 417 - protein, 17 aa, synthetic construct
DPAAFSCHVAAWQCHTL

SEQ ID NO: 418 - protein, 17 aa, synthetic construct
DPALFDCHIAAYQCIFY

SEQ ID NO: 419 - protein, 17 aa, synthetic construct
DPARSDCHVAAYWCYHQ

SEQ ID NO: 420 - protein, 17 aa, synthetic construct
DPATMNCHVAAYFCIVE

SEQ ID NO: 421 - protein, 17 aa, synthetic construct
DPARADCQVAAYWCYHQ

SEQ ID NO: 422 - protein, 17 aa, synthetic construct
DPARTDCHVAAYWCYHQ

SEQ ID NO: 423 - protein, 17 aa, synthetic construct
DPAQLNCHVAAFFCFQS

SEQ ID NO: 424 - protein, 17 aa, synthetic construct
DPARADCHVAAYWCYQQ

SEQ ID NO: 425 - protein, 17 aa, synthetic construct
DPARADCRVAAYWCYHQ

SEQ ID NO: 426 - protein, 17 aa, synthetic construct
DPAAHDCHIAAYWCLNY

SEQ ID NO: 427 - protein, 17 aa, synthetic construct
DPAQHDCHVAAYQCIWE

SEQ ID NO: 428 - protein, 17 aa, synthetic construct
DPARADCHVAAYWCHHQ

SEQ ID NO: 429 - protein, 17 aa, synthetic construct
DPARYSCHVAAYECILN

SEQ ID NO: 430 - protein, 17 aa, synthetic construct
DPARADCHVAAYWCYHE

SEQ ID NO: 431 - protein, 17 aa, synthetic construct
DPAQADCHIAAYLCLFD

SEQ ID NO: 432 - protein, 17 aa, synthetic construct
DPARAACHVAAYWCYHQ

SEQ ID NO: 433 - protein, 17 aa, synthetic construct
DPARADCHVAAYWCYHH

SEQ ID NO: 434 - protein, 11 aa, synthetic construct
PNCHVMAYWCW

SEQ ID NO: 435 - protein, 11 aa, synthetic construct
PNCHVEAYFCM

SEQ ID NO: 436 - protein, 11 aa, synthetic construct
PHDCHVNAWYC

SEQ ID NO: 437 - protein, 11 aa, synthetic construct
PDCHVAAYICY

SEQ ID NO: 438 - protein, 11 aa, synthetic construct
PDCHLNAYWCM

SEQ ID NO: 439 - protein, 11 aa, synthetic construct
PTCHIDAWVCY

SEQ ID NO: 440 - protein, 11 aa, synthetic construct
PFCHIEAYYCW

SEQ ID NO: 441 - protein, 11 aa, synthetic construct
PNCHLYAWECY

SEQ ID NO: 442 - protein, 11 aa, synthetic construct
PDCHIYAFYCL

SEQ ID NO: 443 - protein, 11 aa, synthetic construct
PDCHVAAWMCF

SEQ ID NO: 444 - protein, 11 aa, synthetic construct
PTCHIHAYHCY

SEQ ID NO: 445 - protein, 11 aa, synthetic construct
PDCHIASWFCS

SEQ ID NO: 446 - protein, 11 aa, synthetic construct
PDCHIQAYICH

SEQ ID NO: 447 - protein, 11 aa, synthetic construct
PDCHIRAYVCH

SEQ ID NO: 448 - protein, 11 aa, synthetic construct
PNCHVEAWYCI

SEQ ID NO: 449 - protein, 11 aa, synthetic construct
PDCHIRAYMCN

SEQ ID NO: 450 - protein, 11 aa, synthetic construct
PDCHIAAWYCR

SEQ ID NO: 451 - protein, 11 aa, synthetic construct
PECHVEAYFCE

SEQ ID NO: 452 - protein, 11 aa, synthetic construct
PTCHVQAWYCW

SEQ ID NO: 453 - protein, 11 aa, synthetic construct
PYDCHIAAWMC

SEQ ID NO: 454 - protein, 17 aa, synthetic construct
DPALWQCVFAARSCYEE

SEQ ID NO: 455 - protein, 17 aa, synthetic construct
DPAVWACTFAARYCHEA

SEQ ID NO: 456 - protein, 17 aa, synthetic construct
DPATWRCVFAARACSMQ

SEQ ID NO: 457 - protein, 17 aa, synthetic construct
DPALWMCVFAARQCYES

SEQ ID NO: 458 - protein, 17 aa, synthetic construct
DPAAADCQWAAFLCRVY

SEQ ID NO: 459 - protein, 17 aa, synthetic construct
DPAYQTCWWAAYICAQY

SEQ ID NO: 460 - protein, 17 aa, synthetic construct
DPAVDDCWVAATICRIM

SEQ ID NO: 461 - protein, 17 aa, synthetic construct
DPASEDCRWAAFLCAHY

SEQ ID NO: 462 - protein, 17 aa, synthetic construct
DPAIEECRFAAFQCMYA

SEQ ID NO: 463 - protein, 17 aa, synthetic construct
DPAIADCRFAAFRCMYA

SEQ ID NO: 464 - protein, 17 aa, synthetic construct
DPAVQSCWWAAYTCAIQ

SEQ ID NO: 465 - protein, 17 aa, synthetic construct
DPASSDCSWAAFRCMFS

SEQ ID NO: 466 - protein, 17 aa, synthetic construct
DPAVDYCRFAAFSCQWA

SEQ ID NO: 467 - protein, 17 aa, synthetic construct
DPAIRDCAWAAFLCMHE

SEQ ID NO: 468 - protein, 17 aa, synthetic construct
  SITE 1: note = Acetylated residue
  SITE 17: note = Amidated residue
  SITE 7..14: note = Cysteines are stapled
DPADRRCIQAARVCAVL

SEQ ID NO: 469 - protein, 17 aa, synthetic construct
  SITE 1: note = Acetylated residue
  SITE 17: note = Amidated residue
  SITE 7..14: note = Cysteines are stapled
DPASMRCHRAAHICSVL

SEQ ID NO: 470 - protein, 17 aa, synthetic construct
  SITE 1: note = Acetylated residue
  SITE 17: note = Amidated residue
  SITE 7..14: note = Cysteines are stapled
DPAMYHCEQAANVCRVL

SEQ ID NO: 471 - protein, 17 aa, synthetic construct
  SITE 1: note = Acetylated residue
  SITE 17: note = Amidated residue
  SITE 7..14: note = Cysteines are stapled
DPAQMYCREAAFYCFMH

SEQ ID NO: 472 - protein, 17 aa, synthetic construct
  SITE 1: note = Acetylated residue
  SITE 17: note = Amidated residue
  SITE 7..14: note = Cysteines are stapled
DPATIMCRAAADTCTFF

SEQ ID NO: 473 - protein, 17 aa, synthetic construct
  SITE 1: note = Acetylated residue
  SITE 17: note = Amidated residue
  SITE 7..14: note = Cysteines are stapled
DPAYHHCLAAADLCSFF

SEQ ID NO: 474 - protein, 17 aa, synthetic construct
  SITE 1: note = Acetylated residue
  SITE 17: note = Amidated residue
  SITE 7..14: note = Cysteines are stapled
DPADQWCLAAADVCHFF

SEQ ID NO: 475 - protein, 17 aa, synthetic construct
  SITE 1: note = Acetylated residue
  SITE 17: note = Amidated residue
  SITE 7..14: note = Cysteines are stapled
DPANWECRYAAFNCFIQ

SEQ ID NO: 476 - protein, 17 aa, synthetic construct
  SITE 1: note = Acetylated residue
  SITE 17: note = Amidated residue
  SITE 7..14: note = Cysteines are stapled
DPAAWDCLYAAWDCYIN

SEQ ID NO: 477 - protein, 17 aa, synthetic construct
  SITE 1: note = Acetylated residue
  SITE 17: note = Amidated residue
  SITE 7..14: note = Cysteines are stapled
DPALQACTHAANLCHHF

SEQ ID NO: 478 - protein, 17 aa, synthetic construct
  SITE 1: note = Acetylated residue
  SITE 17: note = Amidated residue
  SITE 7..14: note = Cysteines are stapled
DPALMQCAHAAVLCHQF

SEQ ID NO: 479 - protein, 17 aa, synthetic construct
  SITE 1: note = Acetylated residue
  SITE 17: note = Amidated residue
  SITE 7..14: note = Cysteines are stapled
DPATQRCSMAAYMCHTT

SEQ ID NO: 480 - protein, 17 aa, synthetic construct
  SITE 1: note = Acetylated residue
  SITE 17: note = Amidated residue
  SITE 7..14: note = Cysteines are stapled
DPAWWNCFSAAQQCDAM

SEQ ID NO: 481 - protein, 17 aa, synthetic construct
  SITE 1: note = Acetylated residue
  SITE 17: note = Amidated residue
  SITE 7..14: note = Cysteines are stapled
DPAWYDCADAAWICTFQ

SEQ ID NO: 482 - protein, 17 aa, synthetic construct
  SITE 1: note = Acetylated residue
  SITE 17: note = Amidated residue
  SITE 7..14: note = Cysteines are stapled
DPADRWCELAAWTCDTF

SEQ ID NO: 483 - protein, 10 aa, synthetic construct
  SITE 1: note = Acetylated residue
  SITE 10: note = Amidated residue
  SITE 1..8: note = Cysteines are stapled
CWEAWLLCET

SEQ ID NO: 484 - protein, 11 aa, synthetic construct
  SITE 1: note = Acetylated residue
  SITE 11: note = Amidated residue
  SITE 2..9: note = Cysteines are stapled
PCYEAWVLCEY

SEQ ID NO: 485 - protein, 17 aa, synthetic construct
  SITE 1: note = Acetylated residue
  SITE 17: note = Amidated residue
  SITE 7..14: note = Cysteines are stapled
DPANHACFQAAWDCQFF

SEQ ID NO: 486 - protein, 14 aa, synthetic construct
  SITE 1: note = Acetylated residue
  SITE 4: note = (R)-2-amino-2-methyldec-9-enoic acid, useful for stapling/stapled with another amino acid residue comprising an olefin group to form a staple through the olefin group
  SITE 10: note = (S)-2-amino-3-cyclobutylpropanoic acid
  SITE 11: note = (S)-2-amino-2-methylhept-6-enoic acid, useful for stapling/stapled with another amino acid residue comprising an olefin group to form a staple through the olefin group
  SITE 14: note = Amidated residue
  SITE 4..11: note = Residues are stapled
LTFXEYWAQXXSAA

SEQ ID NO: 487 - protein, 21 aa, synthetic construct
  SITE 1: note = Acetylated residue
  SITE 21: note = Amidated residue
  SITE 8..15: note = Cysteines are stapled
PVPFFWECQYAAATCQTPRIK

SEQ ID NO: 488 - protein, 16 aa, synthetic construct
  SITE 1: note = Acetylated residue
  SITE 16: note = Amidated residue
  SITE 8..15: note = Cysteines are stapled
PVPFFWECQYAAATCQ

SEQ ID NO: 489 - protein, 21 aa, synthetic construct
  SITE 1: note = Acetylated residue
  SITE 21: note = Amidated residue
  SITE 8..15: note = Cysteines are stapled
PTPFFWECQFAAATCTAPRVQ

SEQ ID NO: 490 - protein, 21 aa, synthetic construct
  SITE 1: note = Acetylated residue
  SITE 21: note = Amidated residue
  SITE 8..15: note = Cysteines are stapled
PVPFFWDCQFAAATCDAPQRR

SEQ ID NO: 491 - protein, 32 aa, synthetic construct; identical to SEQ ID NO: 42
  SITE 1: note = Acetylated residue
  SITE 32: note = Amidated residue

SEQ ID NO: 492 - protein, 21 aa, synthetic construct
  SITE 1: note = Acetylated residue
  SITE 21: note = Amidated residue
  SITE 8..15: note = Cysteines are stapled
PAQDDWSCVEAAYLCENYVRV

SEQ ID NO: 493 - protein, 21 aa, synthetic construct
  SITE 1: note = Acetylated residue
  SITE 21: note = Amidated residue
  SITE 8..15: note = Cysteines are stapled
PILQGMACGPAATICWIDGII

SEQ ID NO: 494 - protein, 21 aa, synthetic construct
  SITE 1: note = Acetylated residue
  SITE 21: note = Amidated residue
  SITE 8..15: note = Cysteines are stapled
PDMLAPMCGPAASICWIDGVI

SEQ ID NO: 495 - protein, 17 aa, synthetic construct
  SITE 1: note = Acetylated residue
  SITE 17: note = Amidated residue
  SITE 4..11: note = Cysteines are stapled
QAICFQAAWMCLADDWT

SEQ ID NO: 496 - protein, 21 aa, synthetic construct
  SITE 1: note = Acetylated residue
  SITE 21: note = Amidated residue
  SITE 8..15: note = Cysteines are stapled
PISAANDCFKAAWQCIIWLHQ

SEQ ID NO: 497 - protein, 21 aa, synthetic construct
  SITE 1: note = Acetylated residue
  SITE 21: note = Amidated residue
  SITE 8..15: note = Cysteines are stapled
PSENARDCFWAAWDCLYFIYQ

SEQ ID NO: 498 - protein, 21 aa, synthetic construct
  SITE 1: note = Acetylated residue
  SITE 21: note = Amidated residue
  SITE 8..15: note = Cysteines are stapled
PNLTGADCFPAAWQCLQFLWD

SEQ ID NO: 499 - protein, 20 aa, synthetic construct
FIVSIRCDLAAFYCQNYTNQ

SEQ ID NO: 500 - protein, 17 aa, synthetic construct
DPAQQTCSQAAWVCSIL

SEQ ID NO: 501 - protein, 17 aa, synthetic construct
DPASMRCHRAAHICSVL

SEQ ID NO: 502 - protein, 17 aa, synthetic construct
DPAIQRCSTAAQVCRYL

SEQ ID NO: 503 - protein, 17 aa, synthetic construct
DPAMYHCEQAANVCRVL

SEQ ID NO: 504 - protein, 17 aa, synthetic construct
DPAATLCNQAANVCALL

SEQ ID NO: 505 - protein, 17 aa, synthetic construct
DPARLDCTAAAYVCSVL

SEQ ID NO: 506 - protein, 17 aa, synthetic construct
DPAIMSCMYAAEVCRVL

SEQ ID NO: 507 - protein, 17 aa, synthetic construct
DPALQTCMDAAQVCRLL

SEQ ID NO: 508 - protein, 17 aa, synthetic construct
DPAVRDCSDAAIVCELV

SEQ ID NO: 509 - protein, 17 aa, synthetic construct
DPALASCQQAAWVCEVL

SEQ ID NO: 510 - protein, 17 aa, synthetic construct
DPAMRRCHQAAWVCEVL

SEQ ID NO: 511 - protein, 17 aa, synthetic construct
DPAHTACNQAARVCHTL

SEQ ID NO: 512 - protein, 17 aa, synthetic construct
DPADRRCIQAARVCAVL

SEQ ID NO: 513 - protein, 17 aa, synthetic construct
DPAIFDCEFAAHVCSVL

SEQ ID NO: 514 - protein, 17 aa, synthetic construct
DPARWACTEAAHVCDTL

SEQ ID NO: 515 - protein, 17 aa, synthetic construct
DPARVDCLVAADMCHMF

SEQ ID NO: 516 - protein, 17 aa, synthetic construct
DPATIMCRAAADTCTFF

SEQ ID NO: 517 - protein, 17 aa, synthetic construct
DPAYHHCLAAADLCSFF

SEQ ID NO: 518 - protein, 17 aa, synthetic construct
DPATYYCEAAAETCRFF

SEQ ID NO: 519 - protein, 17 aa, synthetic construct
DPALWECMTAADWCMFM

SEQ ID NO: 520 - protein, 17 aa, synthetic construct
DPADQWCLAAADVCHFF

SEQ ID NO: 521 - protein, 17 aa, synthetic construct
DPARVNCHAAADTCFFF

SEQ ID NO: 522 - protein, 17 aa, synthetic construct
DPALWHCLAAAETCAMF

SEQ ID NO: 523 - protein, 17 aa, synthetic construct
DPAIHSCYAAAETCQFF

SEQ ID NO: 524 - protein, 17 aa, synthetic construct
DPAANYCEAAADTCQFF

SEQ ID NO: 525 - protein, 17 aa, synthetic construct
DPALLHCMAAAQTCSFF

SEQ ID NO: 526 - protein, 17 aa, synthetic construct
DPAQDTCRAAADTCAFF

SEQ ID NO: 527 - protein, 17 aa, synthetic construct
DPAQNMCQAAADVCMFF

SEQ ID NO: 528 - protein, 17 aa, synthetic construct
DPALIHCRAAADTCAFW

SEQ ID NO: 529 - protein, 17 aa, synthetic construct
DPAIQNCRSAADVCMFF

SEQ ID NO: 530 - protein, 17 aa, synthetic construct
DPANWECRYAAFNCFIQ

SEQ ID NO: 531 - protein, 17 aa, synthetic construct
DPAVWECRWAAFQCWLD

SEQ ID NO: 532 - protein, 17 aa, synthetic construct
DPAAWDCLYAAWDCYIN

SEQ ID NO: 533 - protein, 17 aa, synthetic construct
DPATWDCNVAAHRCWFL

SEQ ID NO: 534 - protein, 17 aa, synthetic construct
DPAVWDCRYAAWDCYTN

SEQ ID NO: 535 - protein, 17 aa, synthetic construct
DPATWDCRNAAWLCLLN

SEQ ID NO: 536 - protein, 17 aa, synthetic construct
DPALWECRNAAWQCWMH

SEQ ID NO: 537 - protein, 17 aa, synthetic construct
DPAMWDCRYAALDCYMT

SEQ ID NO: 538 - protein, 17 aa, synthetic construct
DPANWDCQQAAWLCIIQ

SEQ ID NO: 539 - protein, 17 aa, synthetic construct
DPAMWSCRYAAWECWVL

SEQ ID NO: 540 - protein, 17 aa, synthetic construct
DPAIWDCLYAATNCYIN

SEQ ID NO: 541 - protein, 17 aa, synthetic construct
DPATWDCQQAAHHCWVL

SEQ ID NO: 542 - protein, 17 aa, synthetic construct
DPATWDCIWAAERCWRY

SEQ ID NO: 543 - protein, 17 aa, synthetic construct
DPATWACINAAHNCWRN

SEQ ID NO: 544 - protein, 17 aa, synthetic construct
DPAAWDCRYAALACYIH

SEQ ID NO: 545 - protein, 17 aa, synthetic construct
DPALQACTHAANLCHHF

SEQ ID NO: 546 - protein, 17 aa, synthetic construct
DPATHVCTTAAVMCHQT

SEQ ID NO: 547 - protein, 17 aa, synthetic construct
DPATMNCTLAANICHSH

SEQ ID NO: 548 - protein, 17 aa, synthetic construct
DPAFLTCTLAASLCHQD

SEQ ID NO: 549 - protein, 17 aa, synthetic construct
DPATETCAQAAVLCHSD

SEQ ID NO: 550 - protein, 17 aa, synthetic construct
DPAWMACAWAANLCHSS

SEQ ID NO: 551 - protein, 17 aa, synthetic construct
DPAIIRCTSAAMMCHNT

SEQ ID NO: 552 - protein, 17 aa, synthetic construct
DPAHQTCAFAAVLCHQN

SEQ ID NO: 553 - protein, 17 aa, synthetic construct
DPAMIACSHAAQMCHWS

SEQ ID NO: 554 - protein, 17 aa, synthetic construct
DPAFIQCTHAANMCHSS

SEQ ID NO: 555 - protein, 17 aa, synthetic construct
DPAWVSCVRAANLCHVS

SEQ ID NO: 556 - protein, 17 aa, synthetic construct
DPALMQCAHAAVLCHQF

SEQ ID NO: 557 - protein, 17 aa, synthetic construct
DPATQRCSMAAYMCHTT

SEQ ID NO: 558 - protein, 17 aa, synthetic construct
DPAQLSCTVAAHVCHST

SEQ ID NO: 559 - protein, 17 aa, synthetic construct
DPAFHYCTMAANMCHQD

SEQ ID NO: 560 - protein, 17 aa, synthetic construct
DPAWWHCVAAATQCDQR

SEQ ID NO: 561 - protein, 17 aa, synthetic construct
DPAWYACVDAAYFCDYQ

SEQ ID NO: 562 - protein, 17 aa, synthetic construct
DPAWWHCVDAALQCDYY

SEQ ID NO: 563 - protein, 17 aa, synthetic construct
DPAWWDCFNAARRCDDI

SEQ ID NO: 564 - protein, 17 aa, synthetic construct
DPAWWQCFDAAAYCDVH

SEQ ID NO: 565 - protein, 17 aa, synthetic construct
DPAWWYCMDAATMCDQQ

SEQ ID NO: 566 - protein, 17 aa, synthetic construct
DPAWWYCLDAAYDCDHT

SEQ ID NO: 567 - protein, 17 aa, synthetic construct
DPAWWNCIDAAILCDDY

SEQ ID NO: 568 - protein, 17 aa, synthetic construct
DPAWYACYDAARLCDNF

SEQ ID NO: 569 - protein, 17 aa, synthetic construct
DPAWWNCFSAAQQCDAM

SEQ ID NO: 570 - protein, 17 aa, synthetic construct
DPAWYACMDAARVCDRT

SEQ ID NO: 571 - protein, 17 aa, synthetic construct
DPAWHQCFRAAYMCDNH

SEQ ID NO: 572 - protein, 17 aa, synthetic construct
DPAWHACFQAAYICDQA

SEQ ID NO: 573 - protein, 17 aa, synthetic construct
DPAWWSCFAAAHHCDAL

SEQ ID NO: 574 - protein, 17 aa, synthetic construct
DPAWWDCFNAAMRCDIS

SEQ ID NO: 575 - protein, 17 aa, synthetic construct
DPAWRECAHAAWQCYIQ

SEQ ID NO: 576 - protein, 17 aa, synthetic construct
DPAWYDCADAAWICTFQ

SEQ ID NO: 577 - protein, 17 aa, synthetic construct
DPAWAICAEAAWDCFMI

SEQ ID NO: 578 - protein, 17 aa, synthetic construct
DPAYYVCAEAAWNCLFE

SEQ ID NO: 579 - protein, 17 aa, synthetic construct
DPAIQQCAEAAWSCYVE

SEQ ID NO: 580 - protein, 17 aa, synthetic construct
DPAWHMCAEAAWQCAFW

SEQ ID NO: 581 - protein, 17 aa, synthetic construct
DPAWFTCAQAAWDCVFH

SEQ ID NO: 582 - protein, 17 aa, synthetic construct
DPAWFDCAEAAWQCVFI

SEQ ID NO: 583 - protein, 17 aa, synthetic construct
DPAWRACAQAAWDCIIA

SEQ ID NO: 584 - protein, 17 aa, synthetic construct
DPAWQWCAEAAWQCITH

SEQ ID NO: 585 - protein, 17 aa, synthetic construct
DPATFACANAAWECYAR

SEQ ID NO: 586 - protein, 17 aa, synthetic construct
DPAWMRCATAAWECVMT

SEQ ID NO: 587 - protein, 17 aa, synthetic construct
DPAWWECAEAAWQCQVL

SEQ ID NO: 588 - protein, 17 aa, synthetic construct
DPAWMTCATAAWECYLA

SEQ ID NO: 589 - protein, 17 aa, synthetic construct
DPAWYECASAAWVCYSM

SEQ ID NO: 590 - protein, 17 aa, synthetic construct
DPADRWCELAAWTCDTF

SEQ ID NO: 591 - protein, 17 aa, synthetic construct
DPATTECILAAWICDRF

SEQ ID NO: 592 - protein, 17 aa, synthetic construct
DPASRLCELAAWVCDSF

SEQ ID NO: 593 - protein, 17 aa, synthetic construct
DPASRMCEMAAWFCENY

SEQ ID NO: 594 - protein, 17 aa, synthetic construct
DPAMNHCVLAAWVCDTF

SEQ ID NO: 595 - protein, 17 aa, synthetic construct
DPAQRACQFAAWICERF

SEQ ID NO: 596 - protein, 17 aa, synthetic construct
DPADRQCIFAAWMCEAF

SEQ ID NO: 597 - protein, 17 aa, synthetic construct
DPADRYCDMAAWVCERF

SEQ ID NO: 598 - protein, 17 aa, synthetic construct
DPAERHCQLAAWVCHQF

SEQ ID NO: 599 - protein, 17 aa, synthetic construct
DPADRLCELAAWVCQSF

SEQ ID NO: 600 - protein, 17 aa, synthetic construct
DPADRWCELAAWICDEF

SEQ ID NO: 601 - protein, 17 aa, synthetic construct
DPATRHCIFAAWACDRF

SEQ ID NO: 602 - protein, 17 aa, synthetic construct
DPATRMCELAAWTCEVW

SEQ ID NO: 603 - protein, 17 aa, synthetic construct
DPADRQCLFAAWYCEHT

SEQ ID NO: 604 - protein, 17 aa, synthetic construct
DPAYDWCDVAALFCLVE

SEQ ID NO: 605 - protein, 17 aa, synthetic construct
DPAYFACDIAALMCTVH

SEQ ID NO: 606 - protein, 17 aa, synthetic construct
DPAMLQCDMAALWCMVS

SEQ ID NO: 607 - protein, 17 aa, synthetic construct
DPAQILCDLAALYCVVE

SEQ ID NO: 608 - protein, 17 aa, synthetic construct
DPAQNDCEVAAFFCWLD

SEQ ID NO: 609 - protein, 17 aa, synthetic construct
DPAEQECDHAALWCIVA

SEQ ID NO: 610 - protein, 17 aa, synthetic construct
DPAEFVCDWAALACLVD

SEQ ID NO: 611 - protein, 17 aa, synthetic construct
DPAEMQCDYAAILCIVR

SEQ ID NO: 612 - protein, 17 aa, synthetic construct
DPAIIQCDQAALWCVVT

SEQ ID NO: 613 - protein, 17 aa, synthetic construct
DPAEAACDIAALMCIID

SEQ ID NO: 614 - protein, 17 aa, synthetic construct
DPAVMECDWAATICDIF

SEQ ID NO: 615 - protein, 17 aa, synthetic construct
DPAEVHCDMAALYCIIR

SEQ ID NO: 616 - protein, 17 aa, synthetic construct
DPAEQMCDIAAVWCLAH

SEQ ID NO: 617 - protein, 17 aa, synthetic construct
DPADLTCEDAAELCEFW

SEQ ID NO: 618 - protein, 17 aa, synthetic construct
DPAFWYCDEAAEMCEFW

SEQ ID NO: 619 - protein, 17 aa, synthetic construct
DPANIACDDAALLCAFW

SEQ ID NO: 620 - protein, 17 aa, synthetic construct
DPANIACDDAALLCELW

SEQ ID NO: 621 - protein, 17 aa, synthetic construct
DPANIACDDAALLCEFW

SEQ ID NO: 622 - protein, 17 aa, synthetic construct
DPADMYCRDAAELCEFW

SEQ ID NO: 623 - protein, 17 aa, synthetic construct
DPAAFFCDDAAERCEFW

SEQ ID NO: 624 - protein, 17 aa, synthetic construct
DPATYFCYDAAELCDWW

SEQ ID NO: 625 - protein, 17 aa, synthetic construct
DPAITTCLDAAELCDFW

SEQ ID NO: 626 - protein, 17 aa, synthetic construct
DPAAWICEEAAEICEFW

SEQ ID NO: 627 - protein, 17 aa, synthetic construct
DPAYFSCEDAAELCEWY

SEQ ID NO: 628 - protein, 17 aa, synthetic construct
DPADMMCENAALLCEFW

SEQ ID NO: 629 - protein, 17 aa, synthetic construct
DPAMTWCLNAAELCEFW

SEQ ID NO: 630 - protein, 17 aa, synthetic construct
DPAIRMCRDAAELCEFW

SEQ ID NO: 631 - protein, 17 aa, synthetic construct
DPANWVCMDAAELCDFY

SEQ ID NO: 632 - protein, 11 aa, synthetic construct
PCWDAWMLCTD

SEQ ID NO: 633 - protein, 10 aa, synthetic construct
ICYNARLECE

SEQ ID NO: 634 - protein, 10 aa, synthetic construct
ICYTAWLECE

SEQ ID NO: 635 - protein, 10 aa, synthetic construct
ICYSAWLECE

SEQ ID NO: 636 - protein, 11 aa, synthetic construct
PCYSAWITCES

SEQ ID NO: 637 - protein, 10 aa, synthetic construct
ICYNAWLECA

SEQ ID NO: 638 - protein, 10 aa, synthetic construct
ICYNAWFECE

SEQ ID NO: 639 - protein, 11 aa, synthetic construct
PCWNAWMVCSV

SEQ ID NO: 640 - protein, 10 aa, synthetic construct
ICYNAWVECE

SEQ ID NO: 641 - protein, 11 aa, synthetic construct
PCFEAWVMCEI

SEQ ID NO: 642 - protein, 11 aa, synthetic construct
PCYSAWIVCSD

SEQ ID NO: 643 - protein, 11 aa, synthetic construct
PCWNAWLYCSN

SEQ ID NO: 644 - protein, 10 aa, synthetic construct
ICWAAWVSCS

SEQ ID NO: 645 - protein, 11 aa, synthetic construct
PCWSAWLACEL

SEQ ID NO: 646 - protein, 11 aa, synthetic construct
PMCSDAWLFCE

SEQ ID NO: 647 - protein, 11 aa, synthetic construct
PCWEAWLWCSM

SEQ ID NO: 648 - protein, 17 aa, synthetic construct
DPAYASCFSAAWDCQFW

SEQ ID NO: 649 - protein, 17 aa, synthetic construct
DPAFDSCFSAAWDCMYW

SEQ ID NO: 650 - protein, 17 aa, synthetic construct
DPAENECFTAAWDCMFF

SEQ ID NO: 651 - protein, 17 aa, synthetic construct
DPANHACFQAAWDCQFF

SEQ ID NO: 652 - protein, 17 aa, synthetic construct
DPASEACFQAAWDCVSW

SEQ ID NO: 653 - protein, 17 aa, synthetic construct
DPATQQCFQAAWDCQFM

SEQ ID NO: 654 - protein, 17 aa, synthetic construct
DPADNRCFQAAWDCAFW

SEQ ID NO: 655 - protein, 17 aa, synthetic construct
DPADSECFQAASDCQYW

SEQ ID NO: 656 - protein, 17 aa, synthetic construct
DPAINTCQDAAFWCYWS

SEQ ID NO: 657 - protein, 17 aa, synthetic construct
DPAETDCSQAAFWCYWQ

SEQ ID NO: 658 - protein, 17 aa, synthetic construct
DPAHNLCFDAAWWCHWQ

SEQ ID NO: 659 - protein, 17 aa, synthetic construct
DPARVHCNQAAFMCHWV

SEQ ID NO: 660 - protein, 17 aa, synthetic construct
DPALMQCTEAAFWCYWE

SEQ ID NO: 661 - protein, 17 aa, synthetic construct
DPALFSCDSAAFSCYWY

SEQ ID NO: 662 - protein, 17 aa, synthetic construct
DPATESCEDAAFWCHWI

SEQ ID NO: 663 - protein, 17 aa, synthetic construct
DPAYTQCSDAAFMCWWW

SEQ ID NO: 664 - protein, 17 aa, synthetic construct
DPAQHHCQQAAFWCQWW

SEQ ID NO: 665 - protein, 17 aa, synthetic construct
DPARLRCQDAAFACMWY

SEQ ID NO: 666 - protein, 17 aa, synthetic construct
DPARRECMDAAFWCQWV

SEQ ID NO: 667 - protein, 17 aa, synthetic construct
DPAHYECTDAAFECYWF

SEQ ID NO: 668 - protein, 17 aa, synthetic construct
DPAQEDCHAAAFWCIWD

SEQ ID NO: 669 - protein, 17 aa, synthetic construct
DPADIVCQDAAFQCFWW

SEQ ID NO: 670 - protein, 17 aa, synthetic construct
DPAAVQCNTAAFQCYWF

SEQ ID NO: 671 - protein, 17 aa, synthetic construct
DPAFEVCQFAAATCSSN

SEQ ID NO: 672 - protein, 17 aa, synthetic construct
DPAFWSCQFAAATCSHL

SEQ ID NO: 673 - protein, 17 aa, synthetic construct
DPAMWHCQYAAIMCNAN

SEQ ID NO: 674 - protein, 17 aa, synthetic construct
DPAFVRCQFAAASCHTI

SEQ ID NO: 675 - protein, 17 aa, synthetic construct
DPAFEYCQWAAATCQMD

SEQ ID NO: 676 - protein, 17 aa, synthetic construct
DPAFEQCQFAAAMCLHH

SEQ ID NO: 677 - protein, 17 aa, synthetic construct
DPAFTACQFAAAMCDSS

SEQ ID NO: 678 - protein, 17 aa, synthetic construct
DPAFWRCQFAAATCEHT

SEQ ID NO: 679 - protein, 17 aa, synthetic construct
DPAFWQCQFAAATCAHY

SEQ ID NO: 680 - protein, 17 aa, synthetic construct
DPAFERCQLAAAFCYDS

SEQ ID NO: 681 - protein, 17 aa, synthetic construct
DPAFDACQFAAAMCDHQ

SEQ ID NO: 682 - protein, 17 aa, synthetic construct
DPALWNCQYAAVMCTQL

SEQ ID NO: 683 - protein, 17 aa, synthetic construct
DPAFWVCQYAAAMCQDV

SEQ ID NO: 684 - protein, 17 aa, synthetic construct
DPAFDRCQWAAALCMMN

SEQ ID NO: 685 - protein, 17 aa, synthetic construct
DPAFTRCQFAAAVCNFT

SEQ ID NO: 686 - protein, 17 aa, synthetic construct
DPAEYACYHAASICNVD

SEQ ID NO: 687 - protein, 17 aa, synthetic construct
DPAIIWCSQAASICTVD

SEQ ID NO: 688 - protein, 17 aa, synthetic construct
DPAYDVCHFAAHICWID

SEQ ID NO: 689 - protein, 17 aa, synthetic construct
DPANHICHYAAHICDVD

SEQ ID NO: 690 - protein, 17 aa, synthetic construct
DPALEECTQAAHICMVD

SEQ ID NO: 691 - protein, 17 aa, synthetic construct
DPAETFCWDAADICMVD

SEQ ID NO: 692 - protein, 17 aa, synthetic construct
DPAVHNCSQAASICDVD

SEQ ID NO: 693 - protein, 17 aa, synthetic construct
DPAVRWCAAAAAICMVD

SEQ ID NO: 694 - protein, 17 aa, synthetic construct
DPASQWCQRAASICWID

SEQ ID NO: 695 - protein, 17 aa, synthetic construct
DPAELFCTEAADICMVD

SEQ ID NO: 696 - protein, 17 aa, synthetic construct
DPAQYNCYFAANICTVD

SEQ ID NO: 697 - protein, 17 aa, synthetic construct
DPAINMCYRAAQICDVD

SEQ ID NO: 698 - protein, 17 aa, synthetic construct
DPAFFECVRAASICMVD

SEQ ID NO: 699 - protein, 17 aa, synthetic construct
DPAQDNCHYAAHICMVD

SEQ ID NO: 700 - protein, 17 aa, synthetic construct
DPARQYCFQAASICMVD

SEQ ID NO: 701 - protein, 17 aa, synthetic construct
DPAMSNCFMAAWECLIQ

SEQ ID NO: 702 - protein, 17 aa, synthetic construct
DPAYSDCFQAAWTCMIH

SEQ ID NO: 703 - protein, 17 aa, synthetic construct
DPASAECFFAAWQCIAT

SEQ ID NO: 704 - protein, 17 aa, synthetic construct
DPAHRQCFTAAWRCMMT

SEQ ID NO: 705 - protein, 17 aa, synthetic construct
DPADRDCFSAAWACVIR

SEQ ID NO: 706 - protein, 17 aa, synthetic construct
DPASQDCFSAAWQCLQT

SEQ ID NO: 707 - protein, 17 aa, synthetic construct
DPAVTECFQAAWRCMVM

SEQ ID NO: 708 - protein, 17 aa, synthetic construct
DPAQQDCFLAAWDCVWQ

SEQ ID NO: 709 - protein, 17 aa, synthetic construct
DPARQDCFSAAWMCMAI

SEQ ID NO: 710 - protein, 17 aa, synthetic construct
DPAQSDCFQAAWECVAN

SEQ ID NO: 711 - protein, 17 aa, synthetic construct
DPAERNCFTAAWQCMVW

SEQ ID NO: 712 - protein, 17 aa, synthetic construct
DPAAQECFRAAWRCMQD

SEQ ID NO: 713 - protein, 17 aa, synthetic construct
DPASADCFNAAWDCMWI

SEQ ID NO: 714 - protein, 17 aa, synthetic construct
DPAHQQCFMAAWRCVVH

SEQ ID NO: 715 - protein, 17 aa, synthetic construct
DPAYEDCFFAAWNCMMV

SEQ ID NO: 716 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnnnnngcagcaascatttgtatggttgatnnnnnnnnnggtggttctggcgcaggtcgtggttc

SEQ ID NO: 717 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnnnnngcagcaascatttgttggattgatnnnnnnnnnggtggttctggcgcaggtcgtggttc

SEQ ID NO: 718 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnnnnngcagcagagrtttgttggmtttatnnnnnnnnnggtggttctggcgcaggtcgtggttc

SEQ ID NO: 719 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnncatgcagcagatrtktgttggmtttwtnnnnnnnnnggtggttctggcgcaggtcgtggttc

SEQ ID NO: 720 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnntwtgaknnntgtnnnnnngcagcaattakgtgtmttgttnnnnnnnnnnnnggtggttctggcgcaggtcgtggttc

SEQ ID NO: 721 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnttttggnnntgtcagtwtgcagcagcgaygtgtnnnnnnnnnnnnnnnnnnggtggttctggcgcaggtcgtggttc

SEQ ID NO: 722 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnntatnnntgtatggaggcagcastgctgtgtaygnnnnnnnnnnnnnnnggtggttctggcgcaggtcgtggttc

SEQ ID NO: 723 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnntggagctgtgyggaggcagcatwtmtrtgtgaknnnywtnnnnnnnnnggtggttctggcgcaggtcgtggttc

SEQ ID NO: 724 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 70..72: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnastatttggtgtrttgatgcagcannnnnntgtnnnnnnnnnnnnnnnnnnggtggttctggcgcaggtcgtggttc

SEQ ID NO: 725 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnmtgtgtatttgggcagcagatgattgtnnnnnnnnnnnnnnnnnnggtggttctggcgcaggtcgtggttc

SEQ ID NO: 726 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnascgatnnntgtgtgtatgcagcatatttctgtgaknnnnnnnnnnnnnnnggtggttctggcgcaggtcgtggttc

SEQ ID NO: 727 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnnnnngcgagcmtttggtgtrttgatnnnnnnnnnnnnggtggttctggcgcaggtcgtggttc

SEQ ID NO: 728 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgttgggakgcgtggvttnnntgtgaannnnnnnnnnnnnnnggtggttctggcgcaggtcgtggttc

SEQ ID NO: 729 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgttwtnnngcagcatttnnntgtvttnnngatnnnnnnnnnggtggttctggcgcaggtcgtggttc

SEQ ID NO: 730 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnngattgttttnnngcagcatggnnntgtwtknnnnnnnnnnnnnnnggtggttctggcgcaggtcgtggttc

SEQ ID NO: 731 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnsaannnnnntgttwtcaagcagcatggnnntgtvttnnngatnnnnnnnnnggtggttctggcgcaggtcgtggttc

SEQ ID NO: 732 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 82..84: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtttttgggcagcatggnnntgtnnnnnnnnnnnnnnnnnnggtggttctggcgcaggtcgtggttc

SEQ ID NO: 733 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgttttcaggcagcatgggattgtcagtwttggnnnnnnnnnggtggttctggcgcaggtcgtggttc

SEQ ID NO: 734 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgttttcaggcagcatgggattgtcagtwttttnnnnnnnnnggtggttctggcgcaggtcgtggttc

SEQ ID NO: 735 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnngatgcagcatttnnntgtnnntwttatnnnnnnnnnggtggttctggcgcaggtcgtggttc

SEQ ID NO: 736 - DNA, 122 bp, synthetic construct; identical to SEQ ID NO: 24
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine

SEQ ID NO: 737 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 70..72: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnntggnnntgttgggakgcagcannnnnntgtnnngaknnnnnnnnnnnnggtggttctggcgcaggtcgtggttc

SEQ ID NO: 738 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 49..51: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 58..60: note = This region may encompass a codon which encodes an amino acid that is not cysteine, lysine, proline, or glycine
  misc_difference 61..63: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnnnnnnnntgtnnnnnngcagcatttnnntgtnnntggnnnnnnnnnnnnggtggttctggcgcaggtcgtggttc

SEQ ID NO: 739 - DNA, 122 bp, synthetic construct
  source 1..122: mol_type = other DNA
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
  misc_difference 43..45: note = This region may encompass a codon which encodes an amino acid that is not cysteine, lysine, proline, or glycine
  misc_difference 46..48: note = This region may encompass a codon which encodes an amino acid that is not cysteine, lysine, proline, or glycine
  misc_difference 52..54: note = This region may encompass a codon which encodes an amino acid that is not cysteine, lysine, proline, or glycine
  misc_difference 73..75: note = This region may encompass a codon which encodes an amino acid that is not cysteine, lysine, proline, or glycine
  misc_difference 79..81: note = This region may encompass a codon which encodes an amino acid that is not cysteine, lysine, proline, or glycine
  misc_difference 85..87: note = This region may encompass a codon which encodes an amino acid that is not cysteine, lysine, proline, or glycine
  misc_difference 88..90: note = This region may encompass a codon which encodes an amino acid that is not cysteine, lysine, proline, or glycine
  misc_difference 91..93: note = This region may encompass a codon which encodes an amino acid that is not cysteine, lysine, proline, or glycine
  misc_difference 94..96: note = This region may encompass a codon which encodes an amino acid that is not cysteine, lysine, proline, or glycine
catgcccgggtacctttctattctcactctgcgccgnnnnnnnnnnnntggnnntgtaccastgcagcarttnnntgtnnntwtnnnnnnnnnnnnggtggttctggcgcaggtcgtggttc

SEQ ID NO: 740 - protein, 21 aa, synthetic construct
PGPMRAECTVAATICMVDGAL

SEQ ID NO: 741 - protein, 21 aa, synthetic construct
PLIALEDCTVAATICMVDGVV

SEQ ID NO: 742 - protein, 21 aa, synthetic construct
PLQQEHLCSVAATICWIDGRI

SEQ ID NO: 743 - protein, 21 aa, synthetic construct
PQQQIQYCTVAASICMVDGIT

SEQ ID NO: 744 - protein, 21 aa, synthetic construct
PWDNEGFCTVAATICMVDGTV

SEQ ID NO: 745 - protein, 21 aa, synthetic construct
PYSLQTACTVAATICWIDGTI

SEQ ID NO: 746 - protein, 21 aa, synthetic construct
PYEEHTMCSVAATICWIDGPV

SEQ ID NO: 747 - protein, 21 aa, synthetic construct
PKYFTADCSVAATICMVDGSS

SEQ ID NO: 748 - protein, 21 aa, synthetic construct
PIEMMVECTAAATICMVDGWV

SEQ ID NO: 749 - protein, 21 aa, synthetic construct
PMAEPEQCTVAATICMVDGMS

SEQ ID NO: 750 - protein, 21 aa, synthetic construct
PMYMTGQCTVAATICMVDGTH

SEQ ID NO: 751 - protein, 21 aa, synthetic construct
PYRDNVECGVAASICMVDGSS

SEQ ID NO: 752 - protein, 21 aa, synthetic construct
PGPVYQHCTVAASICMVDGIT

SEQ ID NO: 753 - protein, 21 aa, synthetic construct
PMATVQQCTVAATICWIDGII

SEQ ID NO: 754 - protein, 21 aa, synthetic construct
PQYKINHCTVAASICMVDGRA

SEQ ID NO: 755 - protein, 21 aa, synthetic construct
PVPFFWECQYAAAMCQQWYNK

SEQ ID NO: 756 - protein, 21 aa, synthetic construct
PTPFFWDCQFAAAMCTKQPLR

SEQ ID NO: 757 - protein, 21 aa, synthetic construct
PVPFFWECQYAAATCQTPRIK

SEQ ID NO: 758 - protein, 21 aa, synthetic construct
PVPFFWECQYAAATCPLVTAK

SEQ ID NO: 759 - protein, 21 aa, synthetic construct
PTNFYDPCVKAAIRCIVTDFE

SEQ ID NO: 760 - protein, 21 aa, synthetic construct
PTPFFWECQFAAATCTAPRVQ

SEQ ID NO: 761 - protein, 21 aa, synthetic construct
PTPFFWDCQFAAATCYWSSHK

SEQ ID NO: 762 - protein, 21 aa, synthetic construct
PVPFFWECQFAAATCIHVYPA

SEQ ID NO: 763 - protein, 21 aa, synthetic construct
PVPFFWDCQFAAATCDAPQRR

SEQ ID NO: 764 - protein, 21 aa, synthetic construct
PIPFFWECQFAAATCNRWTQW

SEQ ID NO: 765 - protein, 21 aa, synthetic construct
PVPFFWPCQFAAATCAGTATQ

SEQ ID NO: 766 - protein, 21 aa, synthetic construct
PTPFFWECQYAAAMCNWDRTH

SEQ ID NO: 767 - protein, 21 aa, synthetic construct
PVPFFDECAQAAIRCIVTQVD

SEQ ID NO: 768 - protein, 21 aa, synthetic construct
PMPFFWECQFAAATCDRRVNW

SEQ ID NO: 769 - protein, 21 aa, synthetic construct
PVPFFWDCQYAAAMCVAPHRF

SEQ ID NO: 770 - protein, 21 aa, synthetic construct
PNWMYWSCVEAAYLCENYVMV

SEQ ID NO: 771 - protein, 21 aa, synthetic construct
PIDRHWSCVEAAYICENYVLT

SEQ ID NO: 772 - protein, 21 aa, synthetic construct
PTMFSWSCVEAAYLCENYVLI

SEQ ID NO: 773 - protein, 21 aa, synthetic construct
PKMQMWSCVEAAFLCENYVFI

SEQ ID NO: 774 - protein, 21 aa, synthetic construct
PGYEMWSCAEAAFLCENYVWA

SEQ ID NO: 775 - protein, 21 aa, synthetic construct
PHNGKWSCVEAAYICENYVYT

SEQ ID NO: 776 - protein, 21 aa, synthetic construct
PAQDDWSCVEAAYLCENYVRV

SEQ ID NO: 777 - protein, 21 aa, synthetic construct
PEHEQWSCVEAAYLCENYVMT

SEQ ID NO: 778 - protein, 21 aa, synthetic construct
PHVSPWSCVEAAYLCENYVRT

SEQ ID NO: 779 - protein, 21 aa, synthetic construct
PMQRQWSCVEAAYLCENYVMV

SEQ ID NO: 780 - protein, 21 aa, synthetic construct
PPKSSWSCVEAAFICENYVRI

SEQ ID NO: 781 - protein, 21 aa, synthetic construct
PIMETWSCVEAAYLCENYVKS

SEQ ID NO: 782 - protein, 21 aa, synthetic construct
PHIQQWSCAEAAYMCENHVAT

SEQ ID NO: 783 - protein, 21 aa, synthetic construct
PPYHSWSCVEAAYLCENYVRA

SEQ ID NO: 784 - protein, 21 aa, synthetic construct
PPAPSWSCVEAAYLCENYVWV

SEQ ID NO: 785 - protein, 21 aa, synthetic construct
PVRAYAMCGPAASICWIDGII

SEQ ID NO: 786 - protein, 21 aa, synthetic construct
PILQGMACGPAATICWIDGII

SEQ ID NO: 787 - protein, 21 aa, synthetic construct
PDMLAPMCGPAASICWIDGVI

SEQ ID NO: 788 - protein, 21 aa, synthetic construct
PPFHMMMCGPAASICMVDGVV

SEQ ID NO: 789 - protein, 21 aa, synthetic construct
PDMEFITCGPAASICMVDGMT

SEQ ID NO: 790 - protein, 21 aa, synthetic construct
PTINMNNCGPAASICWIDGVV

SEQ ID NO: 791 - protein, 21 aa, synthetic construct
PTSEFVVCGAAASICWIDGKV

SEQ ID NO: 792 - protein, 21 aa, synthetic construct
PMYMTDACGPAATICMVDGVV

SEQ ID NO: 793 - protein, 21 aa, synthetic construct
PYPWMHDCGVAATICWIDGVI

SEQ ID NO: 794 - protein, 21 aa, synthetic construct
PDHRFVYCGPAATICMVDGSV

SEQ ID NO: 795 - protein, 21 aa, synthetic construct
PHPVDYMCGPAATICMVDGYI

SEQ ID NO: 796 - protein, 21 aa, synthetic construct
PMSYTKACGPAASICWIDGFI

SEQ ID NO: 797 - protein, 21 aa, synthetic construct
PWGRPVECGPAATICMVDGKI

SEQ ID NO: 798 - protein, 21 aa, synthetic construct
PDPVHVMCGPAASICMVDGVV

SEQ ID NO: 799 - protein, 21 aa, synthetic construct
PMWQWPECGPAATICWIDGMI

SEQ ID NO: 800 - protein, 21 aa, synthetic construct
PDDPMDLCEYAAEVCWIYIDS

SEQ ID NO: 801 - protein, 21 aa, synthetic construct
PQEGVDLCEYAAEVCWIYIKE

SEQ ID NO: 802 - protein, 21 aa, synthetic construct
PDLPVDICAHAADVCWLYLDH

SEQ ID NO: 803 - protein, 21 aa, synthetic construct
PPWPVDICQHAAEVCWLYLPI

SEQ ID NO: 804 - protein, 21 aa, synthetic construct
PYEPFWLCQFAAATCHRWMEV

SEQ ID NO: 805 - protein, 21 aa, synthetic construct
PIEYIDVCDYAAEICWIYAES

SEQ ID NO: 806 - protein, 21 aa, synthetic construct
PLHPIDVCEYAAEVCWIYLDD

SEQ ID NO: 807 - protein, 21 aa, synthetic construct
PDEPIDLCEHAAEICWLYDQS

SEQ ID NO: 808 - protein, 21 aa, synthetic construct
PQWPVDMCNHAADVCWIYMDY

SEQ ID NO: 809 - protein, 21 aa, synthetic construct
PYEEVDLCEHAADVCWLYLDV

SEQ ID NO: 810 - protein, 21 aa, synthetic construct
PEPGPDICAHAADVCWIYVEE

SEQ ID NO: 811 - protein, 21 aa, synthetic construct
PHSFIDLCEHAADVCWLYGDD

SEQ ID NO: 812 - protein, 21 aa, synthetic construct
PVMPVWSCVEAAYMCEWLLDP

SEQ ID NO: 813 - protein, 21 aa, synthetic construct
PPVKEAICFQAAWQCLSDDWA

SEQ ID NO: 814 - protein, 21 aa, synthetic construct
PIEMQSVCFQAAWTCLSDTWY

SEQ ID NO: 815 - protein, 21 aa, synthetic construct
PIYHTNDCFGAAWYCFSEEWV

SEQ ID NO: 816 - protein, 21 aa, synthetic construct
PFKDERVCFQAAWMCVSDDYN

SEQ ID NO: 817 - protein, 21 aa, synthetic construct
PTNIEQPCFQAAWQCVTDDWT

SEQ ID NO: 818 - protein, 21 aa, synthetic construct
PMEQQAICFQAAWMCLADDWT

SEQ ID NO: 819 - protein, 21 aa, synthetic construct
PWKYEQVCYQAAWQCLSDDWD

SEQ ID NO: 820 - protein, 21 aa, synthetic construct
PNLPQRVCFQAAWLCLSDDWT

SEQ ID NO: 821 - protein, 21 aa, synthetic construct
PRNSENICYQAAWYCLTDDWI

SEQ ID NO: 822 - protein, 21 aa, synthetic construct
PGWKQGICFQAAWMCVADEWI

SEQ ID NO: 823 - protein, 21 aa, synthetic construct
PFMSQEVCFQAAWLCVMDDYV

SEQ ID NO: 824 - protein, 21 aa, synthetic construct
PIKTEHVCYQAAWQCLVDDEN

SEQ ID NO: 825 - protein, 21 aa, synthetic construct
PYVHQDICFQAAWMCVKDEWM

SEQ ID NO: 826 - protein, 21 aa, synthetic construct
PLKLQKVCYQAAWQCLMDDYV

SEQ ID NO: 827 - protein, 21 aa, synthetic construct
PTQFKPDCFGAAFTCIHDMYM

SEQ ID NO: 828 - protein, 21 aa, synthetic construct
PDQHHHDCFGAAWHCLLFLED

SEQ ID NO: 829 - protein, 21 aa, synthetic construct
PAKYQQDCFRAAWGCLEYLYY

SEQ ID NO: 830 - protein, 21 aa, synthetic construct
PEQFQKDCFPAAWQCLVYIYG

SEQ ID NO: 831 - protein, 21 aa, synthetic construct
PISAANDCFKAAWQCIIWLHQ

SEQ ID NO: 832 - protein, 21 aa, synthetic construct
PSENARDCFWAAWDCLYFIYQ

SEQ ID NO: 833 - protein, 21 aa, synthetic construct
PEPYIADCFDAAWRCLMYIYD

SEQ ID NO: 834 - protein, 21 aa, synthetic construct
PKVAPPDCFEAAWNCLYFIYN

SEQ ID NO: 835 - protein, 21 aa, synthetic construct
PHQDKQDCFGAAWTCIEWIYQ

SEQ ID NO: 836 - protein, 21 aa, synthetic construct
PVQDHPDCFFAAWDCLDWLYT

SEQ ID NO: 837 - protein, 21 aa, synthetic construct
PSRAKDDCFKAAWNCLEYIWH

SEQ ID NO: 838 - protein, 21 aa, synthetic construct
PEERKNDCFDAAWECIQYLYD

SEQ ID NO: 839 - protein, 21 aa, synthetic construct
PDQNEEDCYQAAWTCILDLYR

SEQ ID NO: 840 - protein, 21 aa, synthetic construct
PEEWRGDCFSAAWDCLNYIYD

SEQ ID NO: 841 - protein, 21 aa, synthetic construct
PNLTGADCFPAAWQCLQFLWD

SEQ ID NO: 842 - protein, 21 aa, synthetic construct
PKGLQSICYQAAWQCLVDTWD

SEQ ID NO: 843 - protein, 21 aa, synthetic construct
PAWEQMYCFQAAWQCITDIWD

SEQ ID NO: 844 - protein, 21 aa, synthetic construct
PQITKADCFPAAWECIMVIFD

SEQ ID NO: 845 - protein, 21 aa, synthetic construct
PDTQQNTCFQAAWMCVSDDWD

SEQ ID NO: 846 - protein, 21 aa, synthetic construct
PITQESVCFQAAWQCLKDPWE

SEQ ID NO: 847 - protein, 21 aa, synthetic construct
PQWEESVCFQAAWHCLADTWD

SEQ ID NO: 848 - protein, 21 aa, synthetic construct
PLLSQYPCFQAAWQCISDIFD

SEQ ID NO: 849 - protein, 21 aa, synthetic construct
PDPQETWCFQAAWQCVADTWD

SEQ ID NO: 850 - protein, 21 aa, synthetic construct
PMTTQQVCFQAAWQCVGDTWD

SEQ ID NO: 851 - protein, 21 aa, synthetic construct
PEIKEWACFQAAWQCLDDTWD

SEQ ID NO: 852 - protein, 21 aa, synthetic construct
PKIIQSVCFQAAWQCLTDTYF

SEQ ID NO: 853 - protein, 21 aa, synthetic construct
PKTMQPVCFQAAWMCVTDHWD

SEQ ID NO: 854 - protein, 21 aa, synthetic construct
PKLNQQECFQAAWWCVRDPWD

SEQ ID NO: 855 - protein, 21 aa, synthetic construct
PPRHENECIHAADICWLYWLY

SEQ ID NO: 856 - protein, 21 aa, synthetic construct
PYSIPTMCQHAADICWLYWLY

SEQ ID NO: 857 - protein, 21 aa, synthetic construct
PLMLDQECTDAAEVCWIYWLY

SEQ ID NO: 858 - protein, 21 aa, synthetic construct
PLWDDQYCDHAADVCWLYWLY

SEQ ID NO: 859 - protein, 21 aa, synthetic construct
PDWQATHCPHAADVCWLYWLY

SEQ ID NO: 860 - protein, 21 aa, synthetic construct
PDVVEVPCLNAAEVCWLYWLY

SEQ ID NO: 861 - protein, 21 aa, synthetic construct
PWGSTDICVYAAYFCESEGLY

SEQ ID NO: 862 - protein, 21 aa, synthetic construct
PLEQFQQCVDAAEVCWLYWLY

SEQ ID NO: 863 - protein, 21 aa, synthetic construct
PGPQQDKCAQAAEICWLYWLY

SEQ ID NO: 864 - protein, 21 aa, synthetic construct
PETEYPQCQDAAEVCWLYWVY

SEQ ID NO: 865 - protein, 21 aa, synthetic construct
PKLEPSNCNHAADVCWLYWLY

SEQ ID NO: 866 - protein, 21 aa, synthetic construct
PEPEYSACSNAAEICWLYWVY

SEQ ID NO: 867 - protein, 21 aa, synthetic construct
PIADTQLCVEAAEICWLYWLY

SEQ ID NO: 868 - protein, 21 aa, synthetic construct
PTPSSGQCPHAADVCWLYWLY

SEQ ID NO: 869 - protein, 21 aa, synthetic construct
PAQVQHNCPDAAEICWLYWLH

SEQ ID NO: 870 - protein, 18 aa, synthetic construct
  VARIANT 5: note = Any amino acid
  VARIANT 6: note = Any amino acid
  VARIANT 7: note = Any amino acid
  VARIANT 9: note = Any amino acid
  VARIANT 10: note = Any amino acid
  VARIANT 13: note = Any amino acid
  VARIANT 14: note = Any amino acid
  VARIANT 16: note = Any amino acid
  VARIANT 17: note = Any amino acid
  VARIANT 18: note = Any amino acid
  SITE 8..15: note = Cysteines are stapled
ADPAXXXCXXAAXXCXXX

SEQ ID NO: 871 - protein, 14 aa, synthetic construct
HDYCILAAHVCRWD

SEQ ID NO: 872 - protein, 14 aa, synthetic construct
THRCEWAALHCELV

SEQ ID NO: 873 - protein, 14 aa, synthetic construct
VMECYEAAFICHYV

SEQ ID NO: 874 - protein, 14 aa, synthetic construct
IIRCYEAAFYCQNY

SEQ ID NO: 875 - protein, 14 aa, synthetic construct
WQTCAWAAWLCDNH

SEQ ID NO: 876 - protein, 16 aa, synthetic construct
EENPESILDEHVQRMK

SEQ ID NO: 877 - protein, 17 aa, synthetic construct
  SITE 7..14: note = Cysteines are stapled
DPATHRCEWAALHCELV

SEQ ID NO: 878 - protein, 17 aa, synthetic construct
  SITE 7..14: note = Cysteines are stapled
DPAAHRCEWAALHCELV

SEQ ID NO: 879 - protein, 17 aa, synthetic construct
  SITE 7..14: note = Cysteines are stapled
DPATARCEWAALHCELV

SEQ ID NO: 880 - protein, 17 aa, synthetic construct
  SITE 7..14: note = Cysteines are stapled
DPATHACEWAALHCELV

SEQ ID NO: 881 - protein, 17 aa, synthetic construct
  SITE 7..14: note = Cysteines are stapled
DPATHRCAWAALHCELV

SEQ ID NO: 882 - protein, 17 aa, synthetic construct
  SITE 7..14: note = Cysteines are stapled
DPATHRCEAAALHCELV

SEQ ID NO: 883 - protein, 17 aa, synthetic construct
  SITE 7..14: note = Cysteines are stapled
DPATHRCEWAAAHCELV

SEQ ID NO: 884 - protein, 17 aa, synthetic construct
  SITE 7..14: note = Cysteines are stapled
DPATHRCEWAALACELV

SEQ ID NO: 885 - protein, 17 aa, synthetic construct
  SITE 7..14: note = Cysteines are stapled
DPATHRCEWAALHCALV

SEQ ID NO: 886 - protein, 17 aa, synthetic construct
  SITE 7..14: note = Cysteines are stapled
DPATHRCEWAALHCEAV

SEQ ID NO: 887 - protein, 17 aa, synthetic construct
  SITE 7..14: note = Cysteines are stapled
DPATHRCEWAALHCELA

SEQ ID NO: 888 - protein, 17 aa, synthetic construct
  SITE 1: note = Acetylated residue
  SITE 17: note = Amidated residue
  SITE 7..14: note = Cysteines are stapled
DPAWWVCAIAAIECSDV

SEQ ID NO: 889 - protein, 13 aa, synthetic construct
  SITE 1: note = Acetylated residue
  SITE 13: note = Amidated residue
  SITE 4..11: note = Cysteines are stapled
WWVCAIAAIECSD

SEQ ID NO: 890 - protein, 17 aa, synthetic construct
  SITE 7..14: note = Cysteines are stapled
DPALWQCVFAARYCYEE

SEQ ID NO: 891 - protein, 17 aa, synthetic construct
  SITE 7..14: note = Cysteines are stapled
DPALAQCVFAARSCYEE

SEQ ID NO: 892 - protein, 17 aa, synthetic construct
DPAQMYCREAAFYCFMH

SEQ ID NO: 893 - protein, 14 aa, synthetic construct
  VARIANT 1: note = Any amino acid
  VARIANT 2: note = Any amino acid
  VARIANT 3: note = Any amino acid
  VARIANT 5: note = Any amino acid
  VARIANT 6: note = Any amino acid
  VARIANT 9: note = S or T
  VARIANT 13: note = V or I
XXXCXXAAXICMXD

SEQ ID NO: 894 - DNA, 42 bp, synthetic construct
  source 1..42: mol_type = other DNA
  misc_difference 1..3: note = This region may encompass a codon which encodes an amino acid that is not cysteine, lysine, proline, or glycine
  misc_difference 4..6: note = This region may encompass a codon which encodes an amino acid that is not cysteine, lysine, proline, or glycine
  misc_difference 7..9: note = This region may encompass a codon which encodes an amino acid that is not cysteine, lysine, proline, or glycine
  misc_difference 13..15: note = This region may encompass a codon which encodes an amino acid that is not cysteine, lysine, proline, or glycine
  misc_difference 16..18: note = This region may encompass a codon which encodes an amino acid that is not cysteine, lysine, proline, or glycine
nnnnnnnnntgtnnnnnngcggcgascatttgtatgrttgat

SEQ ID NO: 895 - protein, 14 aa, synthetic construct
  VARIANT 1: note = Any amino acid
  VARIANT 2: note = Any amino acid
  VARIANT 6: note = Any amino acid
  VARIANT 10: note = Any amino acid
  VARIANT 12: note = F, L, I, or M
  VARIANT 13: note = Any amino acid
  VARIANT 14: note = Any amino acid
XXDCFXAAWXCXXX

SEQ ID NO: 896 - DNA, 42 bp, synthetic construct
  source 1..42: mol_type = other DNA
  misc_difference 1..3: note = This region may encompass a codon which encodes an amino acid that is not cysteine, lysine, proline, or glycine
  misc_difference 4..6: note = This region may encompass a codon which encodes an amino acid that is not cysteine, lysine, proline, or glycine
  misc_difference 16..18: note = This region may encompass a codon which encodes an amino acid that is not cysteine, lysine, proline, or glycine
  misc_difference 28..30: note = This region may encompass a codon which encodes an amino acid that is not cysteine, lysine, proline, or glycine
  misc_difference 37..39: note = This region may encompass a codon which encodes an amino acid that is not cysteine, lysine, proline, or glycine
  misc_difference 40..42: note = This region may encompass a codon which encodes an amino acid that is not cysteine, lysine, proline, or glycine
nnnnnngattgttttnnngcagcatggnnntgtwtknnnnnn

SEQ ID NO: 897 - protein, 20 aa, synthetic construct
SENARDCFWAAWDCLYFIYQ

SEQ ID NO: 898 - protein, 20 aa, synthetic construct
MKHQEDCFEAAWRCINFLYN

SEQ ID NO: 899 - protein, 20 aa, synthetic construct
EPYIADCFDAAWRCLMYIYD

SEQ ID NO: 900 - protein, 20 aa, synthetic construct
LSDFWECDLAAFICHYVSVI

SEQ ID NO: 901 - protein, 17 aa, synthetic construct
  SITE 7..14: note = Cysteines are stapled
DPAILECHIAAWNCYEI

SEQ ID NO: 902 - protein, 17 aa, synthetic construct
  SITE 7..14: note = Cysteines are stapled
DPATLDCHIAAWDCWDE

SEQ ID NO: 903 - protein, 17 aa, synthetic construct
  SITE 7..14: note = Cysteines are stapled
DPAILACHLAAMDCSDW

SEQ ID NO: 904 - protein, 17 aa, synthetic construct
  SITE 7..14: note = Cysteines are stapled
DPANANCILAAHECRIW

SEQ ID NO: 905 - protein, 17 aa, synthetic construct
  SITE 7..14: note = Cysteines are stapled
DPAQDDCILAAHVCALW

SEQ ID NO: 906 - protein, 17 aa, synthetic construct
  SITE 7..14: note = Cysteines are stapled
DPATHRCEWAALHCELV

SEQ ID NO: 907 - protein, 17 aa, synthetic construct
  SITE 7..14: note = Cysteines are stapled
DPASELCEWAAIHCDLV

SEQ ID NO: 908 - protein, 17 aa, synthetic construct
  SITE 7..14: note = Cysteines are stapled
DPADWECEHAALLCHYW

SEQ ID NO: 909 - protein, 17 aa, synthetic construct
  SITE 7..14: note = Cysteines are stapled
DPALWQCEHAALLCDVH

SEQ ID NO: 910 - protein, 17 aa, synthetic construct
  SITE 7..14: note = Cysteines are stapled
DPAIIHCYEAAFFCQYI

SEQ ID NO: 911 - protein, 17 aa, synthetic construct
  SITE 7..14: note = Cysteines are stapled
DPAVMECYEAAFICHYV

SEQ ID NO: 912 - protein, 17 aa, synthetic construct
  SITE 7..14: note = Cysteines are stapled
DPAEVECMLAAHVCRAF

SEQ ID NO: 913 - protein, 17 aa, synthetic construct
  SITE 7..14: note = Cysteines are stapled
DPALWQCVFAARSCYEE

SEQ ID NO: 914 - protein, 17 aa, synthetic construct
  SITE 7..14: note = Cysteines are stapled
DPALWMCVFAARQCYES

SEQ ID NO: 915 - protein, 31 aa, Crocodylus porosus; identical to SEQ ID NO: 18